(19) United States
(12) Patent Application Publication
(10) Pub. No.: US 2025/0368995 A1
(43) Pub. Date: Dec. 4, 2025

(54) Linkage Modified Oligomeric Compounds and Uses Thereof

(71) Applicant: Ionis Pharmaceuticals, Inc., Carlsbad, CA (US)

(72) Inventors: Brooke A. Anderson, San Diego, CA (US); Xue-Hai Liang, Del Mar, CA (US); William John Drury, III, Oceanside, CA (US); Michael Oestergaard, Carlsbad, CA (US); Michael T. Migawa, Carlsbad, CA (US); Punit P. Seth, Carlsbad, CA (US)

(73) Assignee: Ionis Pharmaceuticals, Inc., Carlsbad, CA (US)

(21) Appl. No.: 19/298,825

(22) Filed: Aug. 13, 2025

Related U.S. Application Data
(63) Continuation of application No. 18/159,465, filed on Jan. 25, 2023.
Application No. 18/159,465 is a continuation of application No. 17/671,924, filed on Feb. 15, 2022, now Pat. No. 11,629,348.
Application No. 17/671,924 is a continuation of application No. PCT/US2020/046561, filed on Aug. 14, 2020.
(60) Provisional application No. 62/887,525, filed on Aug. 15, 2019.
(60) Provisional application No. 62/953,121, filed on Dec. 23, 2019.
(60) Provisional application No. 62/989,442, filed on Mar. 13, 2020.
(60) Provisional application No. 63/050,042, filed on Jul. 9, 2020.

(51) Int. Cl. C12N 15/113

(52) U.S. Cl. CPC C12N 15/113; C12N 2310/11; C12N 2310/314; C12N 2310/315; C12N 2310/321; C12N 2310/341; C12N 2310/351

(57) ABSTRACT

The present disclosure provides oligomeric compounds (including oligomeric compounds that are antisense agents or portions thereof) comprising a modified oligonucleotide having at least one modified internucleoside linking group.

### SEQUENCE LISTING

The present application is being filed along with a Sequence Listing in electronic format. The Sequence Listing is provided as a file entitled CHEM0101SEQ.xml created Dec. 16, 2022, which is 538,869 bytes in size. The information in the electronic format of the sequence listing is incorporated herein by reference in its entirety.

### FIELD

The present disclosure provides oligomeric compounds (including oligomeric compounds that are antisense agents or portions thereof) comprising a modified oligonucleotide having at least one modified internucleoside linking group.

### BACKGROUND

The principle behind antisense technology is that an antisense compound hybridizes to a target nucleic acid and modulates the amount, activity, and/or function of the target nucleic acid. For example, in certain instances, antisense compounds result in altered transcription or translation of a target. Such modulation of expression can be achieved by, for example, target RNA degradation or occupancy-based inhibition. An example of modulation of RNA target function by degradation is RNase H-based degradation of the target RNA upon hybridization with a DNA-like antisense compound.

Another example of modulation of gene expression by target degradation is RNA interference (RNAi). RNAi refers to antisense-mediated gene silencing through a mechanism that utilizes the RNA-induced silencing complex (RISC). An additional example of modulation of RNA target function is by an occupancy-based mechanism such as is employed naturally by microRNA. MicroRNAs are small non-coding RNAs that regulate the expression of protein-coding RNAs. The binding of an antisense compound to a microRNA prevents that microRNA from binding to its messenger RNA targets, and thus interferes with the function of the microRNA. MicroRNA mimics can enhance native microRNA function. Certain antisense compounds alter splicing of pre-mRNA. Another example of modulation of gene expression is the use of antisense compounds in a CRISPR system. Regardless of the specific mechanism, sequence-specificity makes antisense compounds attractive as tools for target validation and gene functionalization, as well as therapeutics to selectively modulate the expression of genes involved in the pathogenesis of disease.

Antisense technology is an effective means for modulating the expression of one or more specific gene products and can therefore prove to be uniquely useful in a number of therapeutic, diagnostic, and research applications. Chemically modified nucleosides may be incorporated into antisense compounds to enhance one or more properties, such as nuclease resistance, tolerability, pharmacokinetics, or affinity for a target nucleic acid.

### SUMMARY

The present disclosure provides oligomeric compounds (including oligomeric compounds that are antisense agents or portions thereof) comprising modified oligonucleotides consisting of linked nucleosides linked through internucleoside linking groups, wherein at least one of the internucleoside linking groups has Formula VIII:

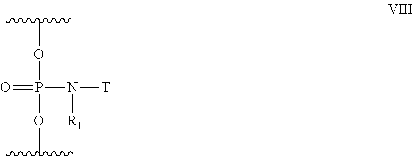

wherein independently for each internucleoside linking group of the modified oligonucleotide having Formula VIII:
R₁ is selected from H, C₁-C₆ alkyl, and substituted C₁-C₆ alkyl; and
T is selected from SO₂R₂, C(═O)R₃, and P(═O)R₄R₅, wherein:
   R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, and substituted C₁-C₆ alkyl;
   R₃ is selected from an aryl, a substituted aryl, CH₃, N(CH₃)₂, and OCH₃;
   R₄ is selected from OCH₃, OH, C₁-C₆ alkyl, and substituted C₁-C₆ alkyl; and
   R₅ is selected from OCH₃, OH, C₁-C₆ alkyl, and substituted C₁-C₆ alkyl.

The present disclosure provides oligomeric compounds (including oligomeric compounds that are antisense agents or portions thereof) comprising modified oligonucleotides consisting of linked nucleosides linked through internucleoside linking groups, wherein at least one of the internucleoside linking groups has Formula VIII:

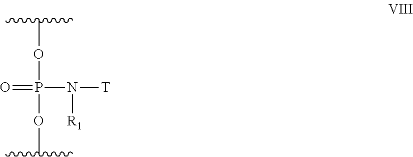

wherein independently for each internucleoside linking group of the modified oligonucleotide having Formula VIII:
R₁ is selected from H, C₁-C₆ alkyl, and substituted C₁-C₆ alkyl; and
T is selected from SO₂R₂, C(═O)R₃, and P(═O)R₄R₅, wherein:
   R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, a C₁-C₆ alkyl, and a substituted C₁-C₆ alkyl;
   R₃ is selected from an aryl, a substituted aryl, CH₃, N(CH₃)₂, and OCH₃;
   R₄ is selected from OCH₃, OH, C₁-C₆ alkyl, and substituted C₁-C₆ alkyl; and
   R₅ is selected from OCH₃, OH, C₁-C₆ alkyl, and substituted C₁-C₆ alkyl;
      provided that if R₁ is H, then T is not:

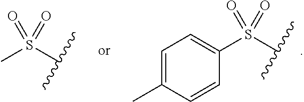

The present disclosure provides oligomeric compounds (including oligomeric compounds that are antisense agents or portions thereof) comprising modified oligonucleotides consisting of linked nucleosides linked through internucleoside linking groups, wherein at least one of the internucleoside linking groups has Formula XVII:

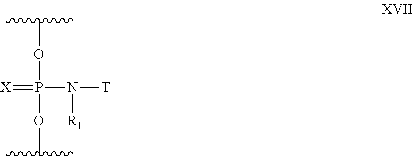

wherein independently for each internucleoside linking group of the modified oligonucleotide having Formula XVII:
X is selected from O or S;
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group; and
   R₅ is selected from OCH₃, OH, C₁-C₆ alkyl, and substituted C₁-C₆ alkyl.

In certain embodiments, oligomeric compounds (including oligomeric compounds that are antisense agents or portions thereof) having at least one internucleoside linking group of Formula VIII or Formula XVII have an increased maximum tolerated dose when administered to an animal compared to an otherwise identical oligomeric compound, except that the otherwise identical oligomeric compound lacks the internucleoside linking group of Formula VIII or Formula XVII.

In certain embodiments, the modified oligonucleotides having at least one internucleoside linking group of Formula VIII or Formula XVII have an increased therapeutic index compared to an otherwise identical oligomeric compound, except that the otherwise identical oligomeric compound lacks the at least one internucleoside linking group of Formula VIII or Formula XVII.

### BRIEF DESCRIPTION OF THE DRAWINGS

FIG. 1 depicts isomers of 2′-deoxyfuranosyl sugar moieties having formulas I-VII.

FIG. 2 depicts isomers of 2′-O-methyl furanosyl sugar moieties having formulas I-VII.

FIG. 3 depicts isomers of 2′-fluoro furanosyl sugar moieties having formulas I-VII.

### DETAILED DESCRIPTION

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, treatises, and GenBank and NCBI reference sequence records are hereby expressly incorporated by reference for the portions of the document discussed herein, as well as in their entirety.

It is understood that the sequence set forth in each SEQ ID NO contained herein is independent of any modification to a sugar moiety, an internucleoside linkage, or a nucleobase. As such, compounds defined by a SEQ ID NO may comprise, independently, one or more modifications to a sugar moiety, an internucleoside linkage, or a nucleobase. Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH(H) sugar moiety and a thymine base could be described as a DNA having a modified sugar (2′-OH in place of one 2′-H of DNA) or as an RNA having a modified base (thymine (methylated uracil) in place of an uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, a modified oligonucleotide having the nucleobase sequence “ATCGATCG” encompasses any modified oligonucleotides having such nucleobase sequence, whether modified or unmodified, including, but not limited to, such compounds comprising RNA bases, such as those having sequence “AUCGAUCG” and those having some DNA bases and some RNA bases such as “AUCGATCG” and modified oligonucleotides having other modified nucleobases, such as “AT^{m}CGAUCG,” wherein ^{m}C indicates a cytosine base comprising a methyl group at the 5-position.

As used herein, “2′-substituted” in reference to a furanosyl sugar moiety or nucleoside comprising a furanosyl sugar moiety means the furanosyl sugar moiety or nucleoside comprising the furanosyl sugar moiety comprises a substituent other than H or OH at the 2′-position and is a non-bicyclic furanosyl sugar moiety. 2′-substituted furanosyl sugar moieties do not comprise additional substituents at other positions of the furanosyl sugar moiety other than a nucleobase and/or internucleoside linkage(s) when in the context of an oligonucleotide.

As used herein, “4′-substituted” in reference to a furanosyl sugar moiety or nucleoside comprising a furanosyl sugar moiety means the furanosyl sugar moiety or nucleoside comprising the furanosyl sugar moiety comprises a substituent other than H at the 4′-position and is a non-bicyclic furanosyl sugar moiety. 4′-substituted furanosyl sugar moieties do not comprise additional substituents at other positions of the furanosyl sugar moiety other than a nucleobase and/or internucleoside linkage(s) when in the context of an oligonucleotide.

As used herein, “5′-substituted” in reference to a furanosyl sugar moiety or nucleoside comprising a furanosyl sugar moiety means the furanosyl sugar moiety or nucleoside comprising the furanosyl sugar moiety comprises a substituent other than H at the 5′-position and is a non-bicyclic furanosyl sugar moiety. 5′-substituted furanosyl sugar moieties do not comprise additional substituents at other positions of the furanosyl sugar moiety other than a nucleobase and/or internucleoside linkage(s) when in the context of an oligonucleotide.

As used herein, “administration” or “administering” refers to routes of introducing a compound or composition provided herein to a subject to perform its intended function. Examples of routes of administration that can be used include, but are not limited to, administration by inhalation, subcutaneous injection, intrathecal injection, and oral administration.

As used herein, “antisense activity” means any detectable and/or measurable change attributable to the hybridization of an antisense oligonucleotide to its target nucleic acid. In certain embodiments, antisense activity is a decrease in the amount or expression of a target nucleic acid or protein encoded by such target nucleic acid compared to target nucleic acid levels or target protein levels in the absence of the antisense oligonucleotide.

As used herein, “antisense agent” means an antisense oligonucleotide or an oligonucleotide duplex comprising an antisense oligonucleotide.

As used herein, “antisense compound” means an antisense oligonucleotide or an oligonucleotide duplex comprising an antisense oligonucleotide.

As used herein, “antisense oligonucleotide” means an oligonucleotide that is complementary to a target nucleic acid and is capable of achieving at least one antisense activity. Antisense oligonucleotides include but are not limited to RNAi antisense modified oligonucleotides and RNase H antisense modified oligonucleotides. In certain embodiments, an antisense oligonucleotide is paired with a sense oligonucleotide to form an oligonucleotide duplex. In certain embodiments, an antisense oligonucleotide is unpaired and is a single-stranded antisense oligonucleotide. In certain embodiments, an antisense oligonucleotide comprises a conjugate group.

As used herein, “artificial mRNA compound” is a modified oligonucleotide, or portion thereof, having a nucleobase sequence comprising one or more codons.

As used herein, “bicyclic nucleoside” or “BNA” means a nucleoside comprising abicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety, and the bicyclic sugar moiety is a modified bicyclic furanosyl sugar moiety. In certain embodiments, the bicyclic sugar moiety does not comprise a furanosyl moiety.

As used herein, “cEt” or “constrained ethyl” or “cEt sugar moiety” means a bicyclic sugar moiety, wherein the first ring of the bicyclic sugar moiety is a ribosyl sugar moiety, the second ring of the bicyclic sugar is formed via a bridge connecting the 4′-carbon and the 2′-carbon, the bridge has the formula 4′-CH(CH₃)—O-2′, and the methyl group of the bridge is in the S configuration. A cEt bicyclic sugar moiety is in the P-D configuration.

As used herein, “complementary” in reference to an oligonucleotide means that at least 70% of the nucleobases of such oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases are nucleobase pairs that are capable of forming hydrogen bonds with one another. Complementary nucleobase pairs include adenine (A) and thymine (T), adenine (A) and uracil (U), cytosine (C) and guanine (G), 5-methyl cytosine (^{m}C) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that such oligonucleotides are complementary to another oligonucleotide or nucleic acid at each nucleoside of the oligonucleotide.

As used herein, “conjugate group” means a group of atoms consisting of a conjugate moiety and a conjugate linker.

As used herein, “conjugate moiety” means a group of atoms that modifies one or more properties of a molecule compared to the identical molecule lacking the conjugate moiety, including but not limited to pharmacodynamics, pharmacokinetics, stability, binding, absorption, tissue distribution, cellular distribution, cellular uptake, charge and clearance.

As used herein, “conjugate linker” means a group of atoms comprising at least one bond.

As used herein, “CRISPR compound” means a modified oligonucleotide that comprises a DNA recognition portion and a tracrRNA recognition portion. As used herein, “DNA recognition portion” is nucleobase sequence that is complementary to a DNA target. As used herein, “tracrRNA recognition portion” is a nucleobase sequence that is bound to or is capable of binding to tracrRNA. The tracrRNA recognition portion of crRNA may bind to tracrRNA via hybridization or covalent attachment.

As used herein, “cytotoxic” or “cytotoxicity” in the context of an effect of an oligomeric compound or a parent oligomeric compound on cultured cells means an at least 2-fold increase in caspase activation following administration of 10 μM or less of the oligomeric compound or parent oligomeric compound to the cultured cells relative to cells cultured under the same conditions but that are not administered the oligomeric compound or parent oligomeric compound. In certain embodiments, cytotoxicity is measured using a standard in vitro cytotoxicity assay.

As used herein, “deoxy region” means a region of 5-12 contiguous nucleotides, wherein at least 70% of the nucleosides are stereo-standard DNA nucleosides. In certain embodiments, each nucleoside is selected from a stereo-standard DNA nucleoside (a nucleoside comprising a β-D-2′-deoxyribosyl sugar moiety), a stereo-non-standard nucleoside of Formula I-VII, a bicyclic nucleoside, and a substituted stereo-standard nucleoside. In certain embodiments, a deoxy region supports RNase H activity. In certain embodiments, a deoxy region is the gap of a gapmer.

As used herein, “double-stranded antisense compound” means an antisense compound comprising two oligomeric compounds that are complementary to each other and form a duplex, and wherein one of the two said oligomeric compounds comprises an antisense oligonucleotide.

As used herein, “expression” includes all the functions by which a gene's coded information is converted into structures present and operating in a cell. Such structures include, but are not limited to, the products of transcription and translation. As used herein, “modulation of expression” means any change in amount or activity of a product of transcription or translation of a gene. Such a change may be an increase or a reduction of any amount relative to the expression level prior to the modulation.

As used herein, “gapmer” means an oligonucleotide having a central region comprising a plurality of nucleosides that support RNase H cleavage positioned between a 5′-region and a 3′-region. Herein, the nucleosides of the 5′-region and 3′-region each comprise a 2′-substituted furanosyl sugar moiety or a bicyclic sugar moiety, and the 3′- and 5′-most nucleosides of the central region each comprise a sugar moiety independently selected from a 2′-deoxyfuranosyl sugar moiety or a sugar surrogate. The positions of the central region refer to the order of the nucleosides of the central region and are counted starting from the 5′-end of the central region. Thus, the 5′-most nucleoside of the central region is at position 1 of the central region. The “central region” may be referred to as a “gap”, and the “5′-region” and “3′-region” may be referred to as “wings”. Gaps of gapmers are deoxy regions.

As used herein, “hepatotoxic” in the context of a mouse means a plasma ALT level that is above 300 units per liter. Hepatotoxicity of an oligomeric compound or parent oligomeric compound that is administered to a mouse is determined by measuring the plasma ALT level of the mouse 24 hours to 2 weeks following at least one dose of 1-150 mg/kg of the compound.

As used herein, “hepatotoxic” in the context of a human means a plasma ALT level that is above 150 units per liter. Hepatotoxicity of an oligomeric compound or parent oligomeric compound that is administered to a human is determined by measuring the plasma ALT level of the human 24 hours to 2 weeks following at least one dose of 10-300 mg of the compound.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “inhibiting the expression or activity” refers to a reduction or blockade of the expression or activity relative to the expression or activity in an untreated or control sample and does not necessarily indicate a total elimination of expression or activity.

As used herein, “internucleoside linkage” or “internucleoside linking group” means a group or bond that forms a covalent linkage between adjacent nucleosides in an oligonucleotide. As used herein “modified internucleoside linkage” means any internucleoside linkage other than a naturally occurring, phosphodiester internucleoside linkage. “Phosphorothioate linkage” means a modified internucleoside linkage in which one of the non-bridging oxygen atoms of a phosphodiester is replaced with a sulfur atom. Modified internucleoside linkages may or may not contain a phosphorus atom. A “neutral internucleoside linkage” is a modified internucleoside linkage that does not have a negatively charged phosphate in a buffered aqueous solution at pH=7.0. A modified internucleoside linkage may optionally comprise a conjugate group.

As used herein, “linked nucleosides” are nucleosides that are connected in a continuous sequence (i.e. no additional nucleosides are present between those that are linked).

As used herein, “maximum tolerated dose” means the highest dose of a compound that does not cause unacceptable side effects. In certain embodiments, the maximum tolerated dose is the highest dose of a modified oligonucleotide that does not cause an ALT elevation of three times the upper limit of normal as measured by a standard assay.

As used herein, “mismatch” or “non-complementary” means a nucleobase of a first oligonucleotide that is not complementary with the corresponding nucleobase of a second oligonucleotide or target nucleic acid when the first and second oligomeric compound are aligned.

As used herein, “modulating” refers to changing or adjusting a feature in a cell, tissue, organ or organism.

As used herein, “MOE” means O-methoxyethyl. “2′-MOE” or “2′-O-methoxyethyl” means a 2′-OCH₂CH₂OCH₃ group at the 2′-position of a furanosyl ring. In certain embodiments, the 2′-OCH₂CH₂OCH₃ group is in place of the 2′-OH group of a ribosyl ring or in place of a 2′-H in a 2′-deoxyribosyl ring. A “2′-MOE sugar moiety” is a sugar moiety with a 2′-OCH₂CH₂OCH₃ group in place of the 2′—OH group of a furanosyl sugar moiety. Unless otherwise indicated, a 2′-MOE sugar moiety is in the P-D ribosyl configuration.

As used herein, a “2′-OMe sugar moiety” is a sugar moiety with a 2′-CH₃ group in place of the 2′—OH group of a furanosyl sugar moiety. Unless otherwise indicated, a 2′-OMe sugar moiety is in the P-D ribosyl configuration and is a “stereo-standard 2′OMe sugar moiety”.

As used herein, a “2′-F sugar moiety” is a sugar moiety with a 2′—F group in place of the 2′—OH group of a furanosyl sugar moiety. Unless otherwise indicated, a 2′-F sugar moiety is in the P-D ribosyl configuration and is a “stereo-standard 2′-F sugar moiety”.

As used herein, “motif” means the pattern of unmodified and/or modified sugar moieties, nucleobases, and/or internucleoside linkages, in an oligonucleotide.

As used herein, “naturally occurring” means found in nature.

As used herein, “nucleobase” means an unmodified nucleobase or a modified nucleobase. As used herein an “unmodified nucleobase” is adenine (A), thymine (T), cytosine (C), uracil (U), or guanine (G). As used herein, a modified nucleobase is a group of atoms capable of pairing with at least one unmodified nucleobase. A universal base is a nucleobase that can pair with any one of the five unmodified nucleobases. 5-methylcytosine (^{m}C) is one example of a modified nucleobase.

As used herein, “nucleobase sequence” means the order of contiguous nucleobases in a nucleic acid or oligonucleotide independent of any sugar moiety or internucleoside linkage modification.

As used herein, “nucleoside” means a moiety comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. A modified nucleoside may comprise a conjugate group.

As used herein, “oligomeric compound” means a compound consisting of (1) an oligonucleotide (a single-stranded oligomeric compound) or two oligonucleotides hybridized to one another (a double-stranded oligomeric compound); and (2) optionally one or more additional features, such as a conjugate group or terminal group which may be attached to the oligonucleotide of a single-stranded oligomeric compound or to one or both oligonucleotides of a double-stranded oligomeric compound.

As used herein, “oligonucleotide” means a strand of linked nucleosides connected via internucleoside linkages, wherein each nucleoside and internucleoside linkage may be modified or unmodified. Unless otherwise indicated, oligonucleotides consist of 12-3000 linked nucleosides, and optionally a conjugate group or terminal group. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “parent antisense agent” means an antisense agent other than an RNAi agent that is identical to an antisense agent having at least one internucleoside linkage of Formula XVII, except that the parent antisense agent has a phosphorothioate internucleoside linkage in place of each internucleoside linkage of Formula XVII in the antisense agent having at least one internucleoside linkage of Formula XVII.

As used herein, “parent RNAi agent” means an RNAi agent that is identical to an RNAi agent having at least one internucleoside linkage of Formula XVII, except that the parent RNAi agent has a phosphodiester internucleoside linkage in place of each internucleoside linkage of Formula XVII in the RNAi agent having at least one internucleoside linkage of Formula XVII.

As used herein, “pharmaceutically acceptable carrier or diluent” means any substance suitable for use in administering to an animal. Certain such carriers enable pharmaceutical compositions to be formulated as, for example, liquids, powders, or suspensions that can be aerosolized or otherwise dispersed for inhalation by a subject. In certain embodiments, a pharmaceutically acceptable carrier or diluent is sterile water; sterile saline; or sterile buffer solution.

As used herein “pharmaceutically acceptable salts” means physiologically and pharmaceutically acceptable salts of compounds, such as oligomeric compounds (including oligomeric compounds that are antisense agents or portions thereof), i.e., salts that retain the desired biological activity of the compound and do not impart undesired toxicological effects thereto.

As used herein “pharmaceutical composition” means a mixture of substances suitable for administering to a subject. For example, a pharmaceutical composition may comprise an antisense compound and an aqueous solution.

As used herein, “RNAi agent” means an antisense agent that acts, at least in part, through RISC or Ago2 to modulate a target nucleic acid and/or protein encoded by a target nucleic acid. RNAi agents include, but are not limited to double-stranded siRNA, single-stranded RNA (ssRNA), and microRNA, including microRNA mimics. RNAi agents may comprise conjugate groups and/or terminal groups. In certain embodiments, an RNAi agent modulates the amount, activity, and/or splicing of a target nucleic acid. The term RNAi agent excludes antisense agents that act through RNase H.

As used herein, “RNAi oligonucleotide” means an RNAi antisense modified oligonucleotide or a RNAi sense modified oligonucleotide.

As used herein, “RNAi antisense modified oligonucleotide” means an oligonucleotide comprising a region that is complementary to a target sequence, and which includes at least one chemical modification suitable for RNAi.

As used herein, “RNAi antisense oligomeric compound” means a single-stranded oligomeric compound comprising a region that is complementary to a target sequence, and which includes at least one chemical modification suitable for RNAi.

As used herein, “RNAi sense modified oligonucleotide” means an oligonucleotide comprising a region that is complementary to a region of an RNAi antisense modified oligonucleotide, and which is capable of forming a duplex with such RNAi antisense modified oligonucleotide.

As used herein, “RNAi sense oligomeric compound” means a single-stranded oligomeric compound comprising a region that is complementary to a region of an RNAi antisense modified oligonucleotide and/or an RNAi antisense oligomeric compound, and which is capable of forming a duplex with such RNAi antisense modified oligonucleotide and/or RNAi antisense oligomeric compound.

A duplex formed by an RNAi antisense modified oligonucleotide and/or an RNAi antisense oligomeric compound with a RNAi sense modified oligonucleotide and/or an RNAi sense oligomeric compound is referred to as a double-stranded RNAi compound (dsRNAi) or a short interfering RNA (siRNA).

As used herein, “RNase H agent” means an antisense agent that acts, at least in part, through RNase H to modulate a target nucleic acid and/or protein encoded by a target nucleic acid. In certain embodiments, RNase H agents are single-stranded. In certain embodiments, RNase H agents are double-stranded. RNase H compounds may comprise conjugate groups and/or terminal groups. In certain embodiments, an RNase H agent modulates the amount or activity of a target nucleic acid. The term RNase H agent excludes antisense agents that act principally through RISC/Ago2.

As used herein, “RNase H antisense modified oligonucleotide” means an oligonucleotide comprising a region that is complementary to a target sequence, and which includes at least one chemical modification suitable for RNase H-mediated nucleic acid reduction.

As used herein, “RNAi compound” means an antisense compound that acts, at least in part, through RISC or Ago2 to modulate a target nucleic acid and/or protein encoded by a target nucleic acid. RNAi compounds include, but are not limited to double-stranded siRNA, single-stranded RNA (ssRNA), and microRNA, including microRNA mimics. In certain embodiments, an RNAi compound modulates the amount, activity, and/or splicing of a target nucleic acid. The term RNAi compound excludes antisense oligonucleotides that act through RNase H.

As used herein, the term “single-stranded” in reference to an antisense compound means such a compound consisting of one oligomeric compound that is not paired with a second oligomeric compound to form a duplex. “Self-complementary” in reference to an oligonucleotide means an oligonucleotide that at least partially hybridizes to itself. A compound consisting of one oligomeric compound, wherein the oligonucleotide of the oligomeric compound is self-complementary, is a single-stranded compound. A single-stranded antisense or oligomeric compound may be capable of binding to a complementary oligomeric compound to form a duplex, in which case the compound would no longer be single-stranded.

As used herein, “stabilized phosphate group” refers to a 5′-chemical moiety that results in stabilization of a 5′-phosphate moiety of the 5′-terminal nucleoside of an oligonucleotide, relative to the stability of an unmodified 5′-phosphate of an unmodified nucleoside under biologic conditions. Such stabilization of a 5′-phophate group includes but is not limited to resistance to removal by phosphatases. Stabilized phosphate groups include, but are not limited to, 5′-vinyl phosphonates and 5′-cyclopropyl phosphonate.

As used herein, “stereo-standard nucleoside” means a nucleoside comprising a non-bicyclic furanosyl sugar moiety having the configuration of naturally occurring DNA and RNA as shown below. A “stereo-standard DNA nucleoside” is a nucleoside comprising a β-D-2′-deoxyribosyl sugar moiety. A “stereo-standard RNA nucleoside” is a nucleoside comprising a β-D-ribosyl sugar moiety. A “substituted stereo-standard nucleoside” is a stereo-standard nucleoside other than a stereo-standard DNA or stereo-standard RNA nucleoside. In certain embodiments, R₁ is a 2′-substituent and R₂—R₅ are each H. In certain embodiments, the 2′-substituent is selected from OMe, F, OCH₂CH₂OCH₃, O-alkyl, SMe, or NMA. In certain embodiments, R₁—R₄ are H and R₅ is a 5′-substituent selected from methyl, allyl, or ethyl. In certain embodiments, the heterocyclic base moiety Bx is selected from uracil, thymine, cytosine, 5-methyl cytosine, adenine or guanine. In certain embodiments, the heterocyclic base moiety Bx is other than uracil, thymine, cytosine, 5-methyl cytosine, adenine or guanine.

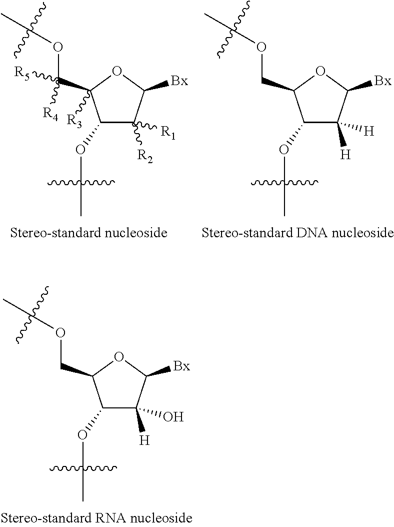

As used herein, “stereo-non-standard nucleoside” means a nucleoside comprising a non-bicyclic furanosyl sugar moiety having a configuration other than that of a stereo-standard sugar moiety. In certain embodiments, a “stereo-non-standard nucleoside” is represented by formulas I-VII below. In certain embodiments, J₁-J₁₄ are independently selected from H, OH, F, OCH₃, OCH₂CH₂OCH₃, O—C₁-C₆ alkoxy, and SCH₃ A “stereo-non-standard RNA nucleoside” has one of formulas I-VII below, wherein each of J₁, J₃, J₅, J₇, J₉, J₁₁, and J₁₃ is H, and each of J₂, J₄, J₆, J₈, J₁₀, J₁₂, and J₁₄ is OH. A “stereo-non-standard DNA nucleoside” has one of formulas I-VII below, wherein each J is H. A “2′-substituted stereo-non-standard nucleoside” has one of formulas I-VII below, wherein either J₁, J₃, J₅, J₇, J₉, J₁₁, and J₁₃ is other than H and/or or J₂, J₄, J₆, J₈, J₁₀, J₁₂, and J₁₄ is other than H or OH. In certain embodiments, the heterocyclic base moiety Bx is selected from uracil, thymine, cytosine, 5-methyl cytosine, adenine or guanine. In certain embodiments, the heterocyclic base moiety Bx is other than uracil, thymine, cytosine, 5-methyl cytosine, adenine or guanine.

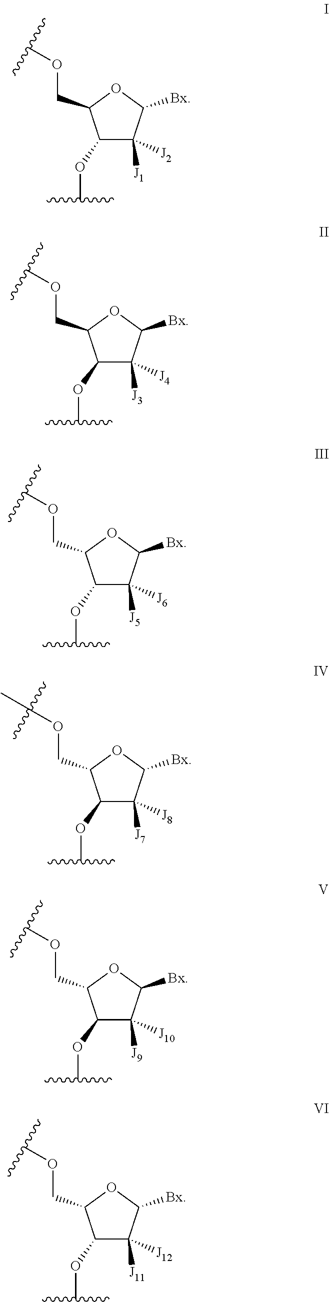

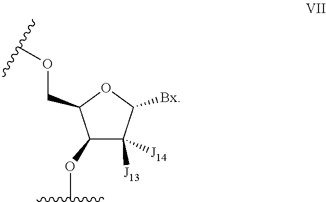

As used herein, “stereo-standard sugar moiety” means the sugar moiety of a stereo-standard nucleoside.

As used herein, “stereo-non-standard sugar moiety” means the sugar moiety of a stereo-non-standard nucleoside.

As used herein, “substituted stereo-non-standard nucleoside” means a stereo-non-standard nucleoside comprising a substituent other than the substituent corresponding to natural RNA or DNA. Substituted stereo-non-standard nucleosides include but are not limited to nucleosides of Formula I-VII wherein the J groups are other than: (1) both H or (2) one H and the other OH.

As used herein, “subject” means a human or non-human animal selected for treatment or therapy.

As used herein, “sugar moiety” means an unmodified sugar moiety or a modified sugar moiety. As used herein, “unmodified sugar moiety” means a β-D-ribosyl moiety, as found in naturally occurring RNA, or a β-D-2′-deoxyribosyl sugar moiety as found in naturally occurring DNA. As used herein, “modified sugar moiety” or “modified sugar” means a sugar surrogate or a furanosyl sugar moiety other than a β-D-ribosyl or a β-D-2′-deoxyribosyl. Modified furanosyl sugar moieties may be modified or substituted at a certain position(s) of the sugar moiety, or unsubstituted, and they may or may not be stereo-non-standard sugar moieties. Modified furanosyl sugar moieties include bicyclic sugars and non-bicyclic sugars. As used herein, “sugar surrogate” means a modified sugar moiety that does not comprise a furanosyl or tetrahydrofuranyl ring (is not a “furanosyl sugar moiety”) and that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

As used herein, “target nucleic acid,” “target RNA,” “target RNA transcript” and “nucleic acid target” means a nucleic acid that an oligomeric compound, such as an antisense compound, is designed to affect. In certain embodiments, an oligomeric compound comprises an oligonucleotide having a nucleobase sequence that is complementary to more than one RNA, only one of which is the target RNA of the oligomeric compound. In certain embodiments, the target RNA is an RNA present in the species to which an oligomeric compound is administered.

As used herein, “therapeutic index” means a comparison of the amount of a compound that causes a therapeutic effect to the amount that causes toxicity. Compounds having a high therapeutic index have strong efficacy and low toxicity. In certain embodiments, increasing the therapeutic index of a compound increases the amount of the compound that can be safely administered.

As used herein, “treat” refers to administering a compound or pharmaceutical composition to an animal in order to effect an alteration or improvement of a disease, disorder, or condition in the animal.

As used herein, “translation suppression element,” means any sequence and/or secondary structure in the 5′-UTR of a target transcript that reduces, inhibits, and/or suppresses translation of the target transcript. In certain embodiments, a translation suppression element comprises a uORF. In certain embodiments, a translation suppression element does not comprise a uORF. In certain embodiments, a translation suppression element comprises one or more stem-loops. In certain embodiments, a translation suppression element comprises greater than 60%, greater than 70%, or greater than 80% GC content. In certain embodiments, the translation suppression element is a uORF. In certain embodiments, the translation suppression element is a stem-loop.

### Certain Embodiments

The present disclosure provides the following non-limiting embodiments:

Embodiment 1. An oligomeric compound comprising a modified oligonucleotide consisting of linked nucleosides linked through internucleoside linking groups, wherein at least one of the internucleoside linking groups has Formula VIII:

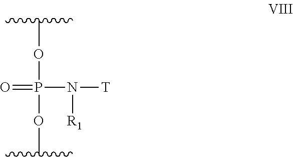

wherein independently for each internucleoside linking group of the modified oligonucleotide having Formula VIII:
R₁ is selected from H, C₁-C₆ alkyl, and substituted C₁-C₆ alkyl; and
T is selected from SO₂R₂, C(═O)R₃, and P(═O)R₄R₅, wherein:
   R₂ is selected from an aryl, a substituted aryl, a heterocycle, a substituted heterocycle, an aromatic heterocycle, a substituted aromatic heterocycle, a diazole, a substituted diazole, an amine, a substituted amine, C₁-C₆ alkoxy, C₁-C₆ alkyl, and substituted C₁-C₆ alkyl;
   R₃ is selected from an aryl, a substituted aryl, CH₃, N(CH₃)₂, and OCH₃;
   R₄ is selected from OCH₃, OH, C₁-C₆ alkyl, and substituted C₁-C₆ alkyl; and
   R₅ is selected from OCH₃, OH, C₁-C₆ alkyl, and substituted C₁-C₆ alkyl.

Embodiment 2. An oligomeric compound comprising a modified oligonucleotide consisting of linked nucleosides linked through internucleoside linking groups, wherein at least one of the internucleoside linking groups has Formula VIII:

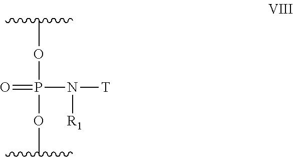

wherein independently for each internucleoside linking group of the modified oligonucleotide having Formula VIII:
R₁ is selected from H, C₁-C₆ alkyl, and substituted C₁-C₆ alkyl; and
T is selected from SO₂R₂, C(═O)R₃, and P(═O)R₄R₅, wherein: R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, a C₁-C₆ alkyl, and a substituted C₁-C₆ alkyl;
   R₃ is selected from an aryl, a substituted aryl, CH₃, N(CH₃)₂, and OCH₃;
   R₄ is selected from OCH₃, OH, C₁-C₆ alkyl, and substituted C₁-C₆ alkyl; and
   R₅ is selected from OCH₃, OH, C₁-C₆ alkyl, and substituted C₁-C₆ alkyl;
      provided that if R₁ is H, then T is not:

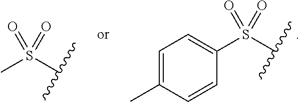

Embodiment 3. The oligomeric compound of embodiment 1 or 2, wherein R₁ is H.

Embodiment 4. The oligomeric compound of embodiment 1 or 2, wherein R₁ is a C₁-C₆ alkyl.

Embodiment 5. The oligomeric compound of embodiment 4, wherein R₁ is methyl.

Embodiment 6. The oligomeric compound of embodiment 1 or 2, wherein R₁ is a substituted C₁-C₆ alkyl;

Embodiment 7. The oligomeric compound of any of embodiments 1-6, wherein T is SO₂R₂.

Embodiment 8. The oligomeric compound of embodiment 7, wherein R₂ is an aryl.

Embodiment 9. The oligomeric compound of embodiment 7, wherein R₂ is a substituted aryl.

Embodiment 10. The oligomeric compound of embodiment 7, wherein R₂ is a heterocycle.

Embodiment 11. The oligomeric compound of embodiment 7, wherein R₂ is a substituted heterocycle.

Embodiment 12. The oligomeric compound of embodiment 7, wherein R₂ is an aromatic heterocycle.

Embodiment 13. The oligomeric compound of embodiment 7, wherein R₂ is a substituted aromatic heterocycle.

Embodiment 14. The oligomeric compound of embodiment 7, wherein R₂ is a diazole.

Embodiment 15. The oligomeric compound of embodiment 7, wherein R₂ is a substituted diazole.

Embodiment 16. The oligomeric compound of embodiment 7, wherein R₂ is an amine.

Embodiment 17. The oligomeric compound of embodiment 7, wherein R₂ is a substituted amine.

Embodiment 18. The oligomeric compound of embodiment 7, wherein R₂ is a C₁-C₆ alkoxy.

Embodiment 19. The oligomeric compound of embodiment 7, wherein R₂ is C₁-C₆ alkyl.

Embodiment 20. The oligomeric compound of embodiment 7, wherein R₂ is substituted C₁-C₆ alkyl.

Embodiment 21. The oligomeric compound of embodiment 7, wherein T is:

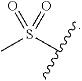 [SMILES: *S(C)(=O)=O]

Embodiment 22. The oligomeric compound of embodiment 7, wherein T is:

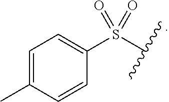 [SMILES: *S(=O)(=O)c1ccc(C)cc1]

Embodiment 23. The oligomeric compound of embodiment 7, wherein T is:

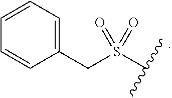 [SMILES: *S(=O)(=O)Cc1ccccc1]

Embodiment 24. The oligomeric compound of embodiment 7, wherein T is:

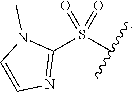 [SMILES: *S(=O)(=O)c1nccn1C]

Embodiment 25. The oligomeric compound of embodiment 7, wherein T is:

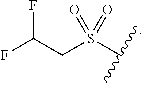 [SMILES: *S(=O)(=O)CC(F)F]

Embodiment 26. The oligomeric compound of embodiment 7, wherein T is:

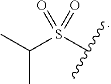 [SMILES: *S(=O)(=O)C(C)C]

Embodiment 27. The oligomeric compound of embodiment 7, wherein T is:

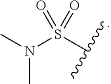 [SMILES: *S(=O)(=O)N(C)C]

Embodiment 28. The oligomeric compound of embodiment 7, wherein T is:

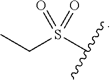 [SMILES: *S(=O)(=O)CC]

Embodiment 29. The oligomeric compound of embodiment 7, wherein T is:

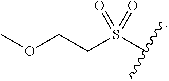 [SMILES: *S(=O)(=O)CCOC]

Embodiment 30. The oligomeric compound of any of embodiments 1-6, wherein T is C(═O)R₃.

Embodiment 31. The oligomeric compound of embodiment 30, wherein R₃ is an aryl.

Embodiment 32. The oligomeric compound of embodiment 30, wherein R₃ is a substituted aryl.

Embodiment 33. The oligomeric compound of embodiment 30, wherein R₃ is CH₃.

Embodiment 34. The oligomeric compound of embodiment 30, wherein R₃ is N(CH₃)₂.

Embodiment 35. The oligomeric compound of embodiment 30, wherein R₃ is OCH₃.

Embodiment 36. The oligomeric compound of embodiment 30, wherein T is:

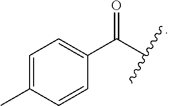 [SMILES: *C(=O)c1ccc(C)cc1]

Embodiment 37. The oligomeric compound of embodiment 30, wherein T is:

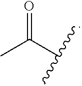 [SMILES: *C(C)=O]

Embodiment 38. The oligomeric compound of embodiment 30, wherein T is:

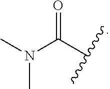 [SMILES: *C(=O)N(C)C]

Embodiment 39. The oligomeric compound of embodiment 30, wherein T is:

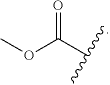 [SMILES: *C(=O)OC]

Embodiment 40. The oligomeric compound of any of embodiments 1-6, wherein T is P(═O)R₄R₅

Embodiment 41. The oligomeric compound of embodiment 40, wherein R₄ is OCH₃.

Embodiment 42. The oligomeric compound of embodiment 40, wherein R₄ is OH

Embodiment 43. The oligomeric compound of embodiment 40, wherein R₄ is C₁-C₆ alkyl.

Embodiment 44. The oligomeric compound of embodiment 40, wherein R₄ is substituted C₁-C₆ alkyl.

Embodiment 45. The oligomeric compound of any of embodiments 40-42, wherein R₅ is OCH₃.

Embodiment 46. The oligomeric compound of any of embodiments 40-42, wherein R₅ is OH.

Embodiment 47. The oligomeric compound of any of embodiments 40-42, wherein R₅ is C₁-C₆ alkyl.

Embodiment 48. The oligomeric compound of any of embodiments 40-42, wherein R₅ is substituted C₁-C₆ alkyl.

Embodiment 49. The oligomeric compound of embodiment 40, wherein T is:

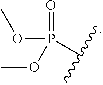 [SMILES: *P(=O)(OC)OC]

Embodiment 50. The oligomeric compound of embodiment 40, wherein T is:

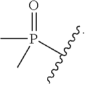 [SMILES: *P(C)(C)=O]

Embodiment 51. The oligomeric compound of any of embodiments 1-50, wherein at least one internucleoside linking group of the modified oligonucleotide is not a linking group of Formula VIII.

Embodiment 52. The oligomeric compound of any of embodiments 1-50, wherein exactly one internucleoside linking group of the modified oligonucleotide is an internucleoside linking group of any of embodiments 1-50.

Embodiment 53. The oligomeric compound of any of embodiments 1-50, wherein exactly two internucleoside linking groups of the modified oligonucleotide are internucleoside linking groups of any of embodiments 1-50.

Embodiment 54. The oligomeric compound of any of embodiments 1-50, wherein exactly three internucleoside linking groups of the modified oligonucleotide are internucleoside linking groups of any of embodiments 1-50.

Embodiment 55. The oligomeric compound of any of embodiments 1-50, wherein exactly four internucleoside linking groups of the modified oligonucleotide are internucleoside linking groups of any of embodiments 1-50.

Embodiment 56. The oligomeric compound of any of embodiments 1-50, wherein exactly five internucleoside linking groups of the modified oligonucleotide are internucleoside linking groups of any of embodiments 1-50.

Embodiment 57. The oligomeric compound of any of embodiments 1-50, wherein at least six internucleoside linking groups of the modified oligonucleotide are internucleoside linking groups of any of embodiments 1-50.

Embodiment 58. The oligomeric compound of any of embodiment 1-51 or 53-57 having at least two linking groups of any of embodiments 1-50, wherein at least two of the linking groups of any of embodiments 1-50 are the same as one another.

Embodiment 59. The oligomeric compound of any of embodiments 1-58, wherein each internucleoside linking group of the modified oligonucleotide that is not an internucleoside linking group of any of embodiments 1-50 is either a phosphodiester internucleoside linking group or a phosphorothioate internucleoside linking group.

Embodiment 60. The oligomeric compound of any of embodiments 1-58, wherein each internucleoside linking group of the modified oligonucleotide is an internucleoside linking group of any of embodiments 1-50.

Embodiment 61. The oligomeric compound of any of embodiments 1-60, wherein at least one nucleoside of the modified oligonucleotide is a modified nucleoside.

Embodiment 62. The oligomeric compound of embodiment 61, wherein at least one nucleoside of the modified oligonucleotide is a modified nucleoside selected from a bicyclic nucleoside and a non-bicyclic substituted nucleoside.

Embodiment 63. The oligomeric compound of any of embodiments 1-62, wherein at least one nucleoside of the modified oligonucleotide is selected from: a β-D-LNA nucleoside, an α-L-LNA nucleoside, an ENA nucleoside, a cEt nucleoside, a 2′-MOE nucleoside, a 2′-OMe nucleoside, a 2′-F nucleoside, a 2′-NMA nucleoside, a 5′-Me nucleoside, a DNA nucleoside, and an RNA nucleoside.

Embodiment 64. The oligomeric compound of any of embodiments 1-62, wherein each nucleoside of the modified oligonucleotide is selected from: a β-D-LNA nucleoside, an α-L-LNA nucleoside, an ENA nucleoside, a cEt nucleoside, a 2′-MOE nucleoside, a 2′-OMe nucleoside, a 2′-F nucleoside, a 2′-NMA nucleoside, a 5′-Me nucleoside, a DNA nucleoside, and an RNA nucleoside.

Embodiment 65. The oligomeric compound of any of embodiments 1-62, wherein at least one nucleoside of the modified oligonucleotide is selected from: a 2′-OMe nucleoside, a 2′-F nucleoside, and an RNA nucleoside.

Embodiment 66. The oligomeric compound of any of embodiments 1-62, wherein at least one nucleoside of the modified oligonucleotide is a 2′-OMe nucleoside, and at least one nucleoside of the modified oligonucleotide is an RNA nucleoside.

Embodiment 67. The oligomeric compound of any of embodiments 61-66, wherein the modified oligonucleotide has a region of alternating nucleoside types having the motif ABABA, wherein each A is a stereo-standard nucleoside of a first type and each B is a stereo-standard nucleoside of a second type, wherein the first type and the second type are different from one another.

Embodiment 68. The oligomeric compound of embodiment 67, wherein A and B are selected from 2′-F substituted nucleosides, 2′-OMe substituted nucleosides, and stereo-standard RNA nucleosides.

Embodiment 69. The oligomeric compound of any of embodiments 1-68, wherein the 5′-end of the modified oligonucleotide comprises a stabilized phosphate group.

Embodiment 70. The oligomeric compound of any of embodiments 1-69, wherein the modified oligonucleotide consists of 12-30 linked nucleosides.

Embodiment 71. The oligomeric compound of any of embodiments 1-69, wherein the modified oligonucleotide consists of 16-24 linked nucleosides.

Embodiment 72. The oligomeric compound of any of embodiments 1-69, wherein the modified oligonucleotide consists of 18-22 linked nucleosides.

Embodiment 73. The oligomeric compound of any of embodiments 1-69, wherein the modified oligonucleotide consists of 16 linked nucleosides.

Embodiment 74. The oligomeric compound of any of embodiments 1-69, wherein the modified oligonucleotide consists of 17 linked nucleosides.

Embodiment 75. The oligomeric compound of any of embodiments 1-69, wherein the modified oligonucleotide consists of 18 linked nucleosides.

Embodiment 76. The oligomeric compound of any of embodiments 1-69, wherein the modified oligonucleotide consists of 19 linked nucleosides.

Embodiment 77. The oligomeric compound of any of embodiments 1-69, wherein the modified oligonucleotide consists of 20 linked nucleosides.

Embodiment 78. The oligomeric compound of any of embodiments 1-69, wherein the modified oligonucleotide consists of 21 linked nucleosides.

Embodiment 79. The oligomeric compound of any of embodiments 1-69, wherein the modified oligonucleotide consists of 22 linked nucleosides.

Embodiment 80. The oligomeric compound of any of embodiments 1-69, wherein the modified oligonucleotide consists of 23 linked nucleosides.

Embodiment 81. The oligomeric compound of any of embodiments 1-80, wherein the oligomeric compound is an RNAi compound.

Embodiment 82. The oligomeric compound of embodiment 81, wherein the RNAi compound is a single-stranded RNAi compound comprising an RNAi antisense modified oligonucleotide, wherein the RNAi antisense modified compound is a modified oligonucleotide of any of embodiments 1-78.

Embodiment 83. The oligomeric compound of embodiment 81, wherein the RNAi compound is a double-stranded RNAi compound comprising an RNAi antisense modified oligonucleotide and an RNAi sense modified oligonucleotide, wherein the RNAi antisense modified oligonucleotide and/or the RNAi sense modified oligonucleotide is a modified oligonucleotide of any of embodiments 1-78.

Embodiment 84. The oligomeric compound of embodiment 82 or 83, wherein at least one internucleoside linking group of the RNAi antisense modified oligonucleotide is an internucleoside linking group of any of embodiments 1-50.

Embodiment 85. The oligomeric compound of embodiment 82 or 83, wherein at least two internucleoside linking groups of the RNAi antisense modified oligonucleotide are independently selected internucleoside linking groups of any of embodiments 1-50.

Embodiment 86. The oligomeric compound of any of embodiments 82-85, wherein at least one of the five 3′-most internucleoside linking groups of the RNAi antisense modified oligonucleotide is an internucleoside linking group of any of embodiments 1-50.

Embodiment 87. The oligomeric compound of any of embodiments 82-86, wherein at least two of the five 3′-most internucleoside linking groups of RNAi antisense modified oligonucleotide is an internucleoside linking group of any of embodiments 1-50.

Embodiment 88. The oligomeric compound of any of embodiments 82-87, wherein at least one internucleoside linking group within the seed region of the RNAi antisense modified oligonucleotide is an internucleoside linking group of any of embodiments 1-50.

Embodiment 89. The oligomeric compound of embodiment 83-88, wherein at least one internucleoside linking group of the RNAi sense modified oligonucleotide is an internucleoside linking group of any of embodiments 1-50.

Embodiment 90. The oligomeric compound of embodiment 89, wherein at least one of the first 5 internucleoside linking groups from the 5′-end of the RNAi sense modified oligonucleotide is an internucleoside linking group of any of embodiments 1-50.

Embodiment 91. The oligomeric compound of any of embodiments 89-90, wherein at least one of the five 3′-most 1 internucleoside linking groups of the RNAi sense modified oligonucleotide is an internucleoside linking group of any of embodiments 1-50.

Embodiment 92. The oligomeric compound of any of embodiments 89-91, wherein at least one of the first 5 internucleoside linking groups from the 5′-end of the RNAi sense modified oligonucleotide and at least one of the five 3′-most linking groups of the RNAi sense modified oligonucleotide is an internucleoside linking group of any of embodiments 1-50.

Embodiment 93. The oligomeric compound of any of embodiments 1-92, wherein at least one nucleoside of the modified oligonucleotide is a stereo-non-standard nucleoside.

Embodiment 94. The oligomeric compound of embodiment 93, wherein the internucleoside linking group linking at least one stereo-non-standard nucleoside to an adjacent nucleoside is an internucleoside linking group of any of embodiments 1-50.

Embodiment 95. The oligomeric compound of embodiment 93 or 94, wherein at least two nucleosides of the modified oligonucleotide are stereo-non-standard nucleosides.

Embodiment 96. The oligomeric compound of embodiment 95, wherein at least two stereo-non-standard nucleosides of the modified oligonucleotide are adjacent to one another.

Embodiment 97. The oligomeric compound of embodiment 96, wherein at least two stereo-non-standard nucleosides of the modified oligonucleotide are linked to one another with an internucleoside linking group of any of embodiments 1-50.

Embodiment 98. The oligomeric compound of any of embodiments 95-97, wherein at least one stereo-non-standard nucleoside of the modified oligonucleotide is a stereo-non-standard DNA nucleoside.

Embodiment 99. The oligomeric compound of any of embodiments 95-97, wherein at least one stereo-non-standard nucleoside of the modified oligonucleotide is a substituted stereo-non-standard nucleoside or a stereo-non-standard RNA nucleoside.

Embodiment 100. The oligomeric compound of embodiment 99, wherein the 2′-substituent of the at least one substituted stereo-non-standard nucleoside of the modified oligonucleotide is selected from: 2′-MOE, 2′-OMe, 2′-F, or 2′-OH.

Embodiment 101. The oligomeric compound of any of embodiments 1-100, wherein the modified oligonucleotide comprises a deoxy region consisting of 6-11 linked nucleosides wherein each nucleoside of the deoxy region is either a modified nucleoside or a stereo-standard DNA nucleoside and wherein at least 3 contiguous nucleosides of the deoxy region are stereo-standard DNA nucleosides and not more than three nucleosides of the deoxy region are modified nucleosides.

Embodiment 102. The oligomeric compound of embodiment 101, wherein at least 4 contiguous nucleosides of the deoxy region are stereo-standard DNA nucleosides.

Embodiment 103. The oligomeric compound of embodiment 101, wherein at least 5 contiguous nucleosides of the deoxy region are stereo-standard DNA nucleosides.

Embodiment 104. The oligomeric compound of embodiment 101, wherein at least 6 contiguous nucleosides of the deoxy region are stereo-standard DNA nucleosides.

Embodiment 105. The oligomeric compound of embodiment 101, wherein at least 7 contiguous nucleosides of the deoxy region are stereo-standard DNA nucleosides.

Embodiment 106. The oligomeric compound of embodiment 101, wherein at least 8 contiguous nucleosides of the deoxy region are stereo-standard DNA nucleosides.

Embodiment 107. The oligomeric compound of any of embodiments 101-106, wherein the deoxy region consists of 8-10 linked nucleosides.

Embodiment 108. The oligomeric compound of any of embodiments 101-106, wherein the deoxy region consists of 9 linked nucleosides.

Embodiment 109. The oligomeric compound of any of embodiments 101-106, wherein the deoxy region consists of 10 linked nucleosides.

Embodiment 110. The oligomeric compound of any of embodiments 101-106, wherein the deoxy region consists of 11 linked nucleosides.

Embodiment 111. The oligomeric compound of any of embodiments 101-110 wherein at least 6 nucleosides of the deoxy region are stereo-standard DNA nucleosides.

Embodiment 112. The oligomeric compound of any of embodiments 101-110 wherein at least 7 nucleosides of the deoxy region are stereo-standard DNA nucleosides.

Embodiment 113. The oligomeric compound of any of embodiments 101-110 wherein at least 8 nucleosides of the deoxy region are stereo-standard DNA nucleosides.

Embodiment 114. The oligomeric compound of any of embodiments 101-110 wherein at least 9 nucleosides of the deoxy region are stereo-standard DNA nucleosides.

Embodiment 115. The oligomeric compound of any of embodiments 101-114 wherein two nucleosides of the deoxy region are modified nucleosides.

Embodiment 116. The oligomeric compound of any of embodiments 101-114 wherein one nucleoside of the deoxy region is a modified nucleoside.

Embodiment 117. The oligomeric compound of any of embodiments 101-116 wherein at least one modified nucleoside of the deoxy region is a stereo-standard modified nucleoside or bicyclic nucleoside selected from a β-D-LNA nucleoside, an α-L-LNA nucleoside, an ENA nucleoside, a cEt nucleoside, a 2′-MOE nucleoside, a 2′-OMe nucleoside, a 2′-F nucleoside, and a 5′-alkyl nucleoside.

Embodiment 118. The oligomeric compound of any of embodiments 101-117 wherein at least one modified nucleoside of the deoxy region is stereo-non-standard nucleoside.

Embodiment 119. The oligomeric compound of embodiment 118 wherein the at least one is stereo-non-standard isomeric nucleoside of the deoxy region is a stereo-non-standard DNA nucleoside.

Embodiment 120. The oligomeric compound of embodiment 119 wherein the stereo-non-standard DNA nucleoside is selected from a stereo-non-standard DNA nucleoside having: Formula I, Formula II, Formula III, Formula IV, Formula V, Formula VI, and Formula VII.

Embodiment 121. The oligomeric compound of embodiment 120 wherein the stereo-non-standard DNA nucleoside is selected from a stereo-non-standard DNA nucleoside having: Formula V and Formula II.

Embodiment 122. The oligomeric compound of any of embodiments 118-121 wherein at least one stereo-non-standard nucleoside of the deoxy region is a substituted stereo-non-standard nucleoside.

Embodiment 123. The oligomeric compound of embodiment 122 wherein at least one substituted stereo-non-standard nucleoside has a 2′-substituent selected from: 2′-MOE, 2′-OMe, 2′-F, or 2′-OH.

Embodiment 124. The oligomeric compound of any of embodiments 101-123, wherein the 2^{nd} nucleoside from the 5′-end of the deoxy region is a modified nucleoside.

Embodiment 125. The oligomeric compound of any of embodiments 101-124, wherein the 3^{rd} nucleoside from the 5′-end of the deoxy region is a modified nucleoside.

Embodiment 126. The oligomeric compound of any of embodiments 101-125, wherein the 4^{th} nucleoside from the 5′-end of the deoxy region is a modified nucleoside.

Embodiment 127. The oligomeric compound of any of embodiments 101-126, wherein each nucleoside of the deoxy region is a stereo-standard DNA nucleoside.

Embodiment 128. The oligomeric compound of any of embodiments 101-127 wherein at least one internucleoside linking group within the deoxy region is an internucleoside linking group of any of embodiments 1-50.

Embodiment 129. The oligomeric compound of any of embodiments 101-128, wherein the internucleoside linking group linking the 1^{st} and 2^{nd} nucleosides of the deoxy region as counted from the 5′-end of the deoxy region is an internucleoside linking group of any of embodiments 1-50.

Embodiment 130. The oligomeric compound of any of embodiments 101-129, wherein the internucleoside linking group linking the 2^{nd} and 3^{rd} nucleosides of the deoxy region as counted from the 5′-end of the deoxy region is an internucleoside linking group of any of embodiments 1-50.

Embodiment 131. The oligomeric compound of any of embodiments 101-130, wherein the internucleoside linking group linking the 3^{rd} and 4^{th} nucleosides of the deoxy region as counted from the 5′-end of the deoxy region is an internucleoside linking group of any of embodiments 1-50.

Embodiment 132. The oligomeric compound of any of embodiments 101-131, wherein the internucleoside linking group linking the 4^{th} and 5^{th} nucleosides of the deoxy region as counted from the 5′-end of the deoxy region is an internucleoside linking group of any of embodiments 1-50.

Embodiment 133. The oligomeric compound of any of embodiments 101-132 wherein one internucleoside linking group in the deoxy region is a linking group of any of embodiments 1-50 and the other internucleoside linking groups of the deoxy region are each phosphodiester or phosphorothioate internucleoside linking groups.

Embodiment 134. The oligomeric compound of any of embodiments 101-133 wherein two internucleoside linking groups in the deoxy region are linking groups of any of embodiments 1-50 and the other internucleoside linking groups of the deoxy region are each phosphodiester or phosphorothioate internucleoside linking groups.

Embodiment 135. The oligomeric compound of any of embodiments 101-134 wherein three internucleoside linking groups in the deoxy region are linking groups of any of embodiments 1-50 and the other internucleoside linking groups of the deoxy region are each phosphodiester or phosphorothioate internucleoside linking groups.

Embodiment 136. The oligomeric compound of any of embodiments 101-135 wherein the deoxy region is flanked on the 5′ side by a 5′-region consisting of 1-6 linked 5′-region nucleosides and on the 3′ side by a 3′-region consisting of 1-6 linked 3′-region nucleosides; wherein
the 3′-most nucleoside of the 5′-region is a modified nucleoside; and
the 5′-most nucleoside of the 3′-region is a modified nucleoside.

Embodiment 137. The oligomeric compound of embodiment 136, wherein at least one 5′-region nucleoside is a stereo-standard DNA nucleoside.

Embodiment 138. The oligomeric compound of embodiment 136, wherein each 5′-region nucleoside is a modified nucleoside.

Embodiment 139. The oligomeric compound of any of embodiments 136, wherein at least one 5′-region nucleoside is a 2′-substituted nucleoside.

Embodiment 140. The oligomeric compound of any of embodiments 136, or 138-139 wherein each 5′-region nucleoside is a 2′-substituted nucleoside.

Embodiment 141. The oligomeric compound of any of embodiments 139-140, wherein the 2′-substitutent is selected from among 2′-F, 2′-OCH₃, and 2′-MOE.

Embodiment 142. The oligomeric compound of any of embodiments 136-139 or 141, wherein at least one 5′-region nucleoside is abicyclic nucleoside.

Embodiment 143. The oligomeric compound of embodiment 142, wherein each 5′-region nucleoside is abicyclic nucleoside.

Embodiment 144. The oligomeric compound of any of embodiments 142-143, wherein the bicyclic 5′-region nucleoside is selected from among a β-D-LNA nucleoside, an α-L-LNA nucleoside, an ENA nucleoside, and a cEt nucleoside.

Embodiment 145. The oligomeric compound of any of embodiments embodiment 136-144, wherein at least one 3′-region nucleoside is a stereo-standard DNA nucleoside.

Embodiment 146. The oligomeric compound of any of embodiments 136-144 wherein each 3′-region nucleoside is a modified nucleoside.

Embodiment 147. The oligomeric compound of any of embodiments 136-146, wherein at least one 3′-region nucleoside is a 2′-substituted nucleoside.

Embodiment 148. The oligomeric compound of any of embodiments 136-144 or 146-147, wherein each 3′-region nucleoside is a 2′- substituted nucleoside.

Embodiment 149. The oligomeric compound of embodiment 147 or 148, wherein the 2′-substituent is selected from among 2′-F, 2′-OCH₃, and 2′-MOE.

Embodiment 150. The oligomeric compound of any of embodiments 136-147 or 149, wherein at least one 3′-region nucleoside is abicyclic nucleoside.

Embodiment 151. The oligomeric compound of embodiment 150, wherein each 3′-region nucleoside is a bicyclic nucleoside.

Embodiment 152. The oligomeric compound of any of embodiments 150-151 wherein the bicyclic 3′-region nucleoside is selected from among a β-D-LNA nucleoside, an α-L-LNA nucleoside, an ENA nucleoside, and a cEt nucleoside.

Embodiment 153. The oligomeric compound of any of embodiments 101-152 wherein the modified oligonucleotide is a gapmer.

Embodiment 154. The oligomeric compound of any of embodiments 1-80 wherein each nucleoside of the modified oligonucleotide is a modified nucleoside and each modified nucleoside of the modified oligonucleotide comprises the same modification.

Embodiment 155. The oligomeric compound of any of embodiments 1-153, wherein the nucleobase sequence of the modified oligonucleotide is complementary to a target nucleic acid.

Embodiment 156. The oligomeric compound of embodiment 155, wherein the nucleobase sequence of the modified oligonucleotide is at least 80% complementary to the target nucleic acid.

Embodiment 157. The oligomeric compound of embodiment 155, wherein the nucleobase sequence of the modified oligonucleotide is at least 85% complementary to the target nucleic acid.

Embodiment 158. The oligomeric compound of embodiment 155, wherein the nucleobase sequence of the modified oligonucleotide is at least 90% complementary to the target nucleic acid.

Embodiment 159. The oligomeric compound of embodiment 155, wherein the nucleobase sequence of the modified oligonucleotide is at least 95% complementary to the target nucleic acid.

Embodiment 160. The oligomeric compound of embodiment 155, wherein the nucleobase sequence of the modified oligonucleotide is 100% complementary to the target nucleic acid.

Embodiment 161. The oligomeric compound of any of embodiments 155-160, wherein the target nucleic acid is a target RNA.

Embodiment 162. The oligomeric compound of embodiment 161, wherein the target RNA is selected from: an mRNA, a pre-mRNA, a microRNA, and a non-coding RNA.

Embodiment 163. The oligomeric compound of embodiment 161, wherein the target RNA is not a microRNA.

Embodiment 164. The oligomeric compound of any of embodiments 1-162, wherein the modified oligonucleotide is not complementary to miR-21.

Embodiment 165. The oligomeric compound of any of embodiments 1-163, comprising a conjugate group.

Embodiment 166. The oligomeric compound of embodiment 164, wherein the conjugate group comprises at least one GalNAc.

Embodiment 167. The oligomeric compound of embodiment 164 or 165, wherein the conjugate group comprises 1-5 linker-nucleosides.

Embodiment 168. The oligomeric compound of any of embodiments 1-80, wherein the oligomeric compound is a CRISPR compound.

Embodiment 169. The oligomeric compound of embodiment 168, wherein the CRISPR compound consists of 20-50 linked nucleosides.

Embodiment 170. The oligomeric compound of embodiment 168, wherein the CRISPR compound consists of 29-32 linked nucleosides.

Embodiment 171. A pharmaceutical composition comprising the CRISPR compound of embodiments 169-170 and a pharmaceutically acceptable carrier or diluent.

Embodiment 172. A method comprising contacting a cell with the CRISPR compound or composition of any of embodiments 169-170.

Embodiment 173. The method of embodiment 172, comprising contacting the cell with a plasmid that encodes Cas9 or Cpf1.

Embodiment 174. The method of embodiment 172-173, wherein the plasmid encodes a tracrRNA.

Embodiment 175. The method of embodiment 174, comprising contacting the cell with an mRNA that encodes Cas9 or Cpf1.

Embodiment 176. The method of any of embodiments 172-175, comprising contacting the cell with a plasmid that encodes a tracrRNA.

Embodiment 177. The method of any of embodiments 172-176 wherein a target gene is edited.

Embodiment 178. The oligomeric compound of any of embodiments 1-80, wherein the oligomeric compound is an artificial mRNA compound.

Embodiment 179. The artificial mRNA compound of embodiment 178, wherein the artificial mRNA oligonucleotide consists of 17-3000 linked nucleosides.

Embodiment 180. The artificial mRNA compound of embodiment 178 or 179, wherein the artificial mRNA oligonucleotide encodes a protein.

Embodiment 181. A pharmaceutical composition comprising the artificial mRNA compound of any of embodiments 178-180 and a pharmaceutically acceptable carrier or diluent.

Embodiment 182. A method comprising contacting a cell with the artificial mRNA compound or composition of any of embodiments 178-181.

Embodiment 183. A pharmaceutical composition comprising the oligomeric compound of any of embodiments 1-182 and a pharmaceutically acceptable carrier or diluent.

Embodiment 184. A method comprising contacting a cell with the oligomeric compound or pharmaceutical composition of any of embodiments 1-167 or 183.

Embodiment 185. A method of modulating the amount or activity of a target nucleic acid in a cell, comprising contacting the cell with the oligomeric compound or pharmaceutical composition of any of embodiments 1-167 or 183, and thereby modulating the amount or activity of the target nucleic acid.

Embodiment 186. A method of modulating the amount or activity of a target nucleic acid in a cell, comprising contacting the cell with the oligomeric compound or pharmaceutical composition of any of embodiments 1-167 or 183.

Embodiment 187. The method of embodiment 186, wherein the amount or activity of a target nucleic acid is reduced.

Embodiment 188. Use of the oligomeric compound or composition of any of embodiments 1-171, 178-181 or 183 for treatment of a disease or condition.

Embodiment 189. Use of the oligomeric compound or composition of any of embodiments 1-171, 178-181 or 183 for a preparation of a medicament for treatment of a disease or condition.

Embodiment 190. An oligomeric compound comprising a modified oligonucleotide consisting of 12-23 linked nucleosides, wherein the modified oligonucleotide comprises a 5′-region, a central region, and a 3′- region wherein:
the 5′-region consists of 1-5 linked nucleosides; wherein at least one 5′-region nucleoside is modified;
the 3′-region consists of 1-5 linked nucleosides; wherein at least one 3′-region nucleoside is modified; and
the central region consists of 7-11 linked nucleosides, and has the formula:
(N_{d1})_{L1}(N_{d2})_{L2}(N_{d3})_{L3}(N_{d4})_{L4}[(N_{d})_{L5}]_{q};
wherein N_{d1}, N_{d2}, N_{d3}, N_{d4} are independently selected from among
a stereo-standard DNA nucleoside, a stereo-non-standard DNA nucleoside, or a 2′-substituted nucleoside; with
the proviso that no more than one of N_{d1}, N_{d2}, N_{d3}, or N_{d4} is a 2′-substituted nucleoside;
each N_{d} is independently selected from among a stereo-standard DNA nucleoside and a stereo-non-standard
DNA nucleoside;
q is from 3-8;
wherein each of L₁, L₂, L₃, L₄, and each L₅ is an internucleoside linkage;
wherein at least two of L₁, L₂, L₃, and L₄ are internucleoside linkages having formula VIII:

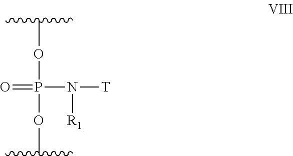

wherein independently for each internucleoside linking group of the modified oligonucleotide having Formula VIII:
R₁ is selected from H, C₁-C₆ alkyl, and substituted C₁-C₆ alkyl; and
T is selected from SO₂R₂, C(═O)R₃, and P(═O)R₄R₅, wherein:
   R₂ is selected from an aryl, a substituted aryl, a heterocycle, a substituted heterocycle, an aromatic heterocycle, a substituted aromatic heterocycle, a diazole, a substituted diazole, an amine, a substituted amine, C₁-C₆ alkoxy, C₁-C₆ alkyl, and substituted C₁-C₆ alkyl;
   R₃ is selected from an aryl, a substituted aryl, CH₃, N(CH₃)₂, and OCH₃;
   R₄ is selected from OCH₃, OH, C₁-C₆ alkyl, and substituted C₁-C₆ alkyl; and
   R₅ is selected from OCH₃, OH, C₁-C₆ alkyl, and substituted C₁-C₆ alkyl.

Embodiment 191. The oligomeric compound of embodiment 190, wherein one of N_{d1}, N_{d2}, N_{d3}, or N_{d4} is a 2′-substituted nucleoside.

Embodiment 192. The oligomeric compound of embodiment 191, wherein the 2′-substituted nucleoside is a 2′-OMe nucleoside.

Embodiment 193. The oligomeric compound of embodiment 191, wherein the 2′-OMe nucleoside is a stereo-standard 2′-OMe nucleoside.

Embodiment 194. The oligomeric compound of any of embodiments 191-193, wherein the 2′-substituted nucleoside is N_{d2}.

Embodiment 195. The oligomeric compound of embodiment 190, wherein each of N_{d1}, N_{d2}, N_{d3}, N_{d4} and each Na is a DNA nucleoside.

Embodiment 196. The oligomeric compound of embodiment 195, wherein each DNA nucleoside is a stereo-standard DNA nucleoside.

Embodiment 197. The oligomeric compound of any of embodiments 190-196, wherein L₁ and L₂ are internucleoside linkages having formula VIII.

Embodiment 198. The oligomeric compound of any of embodiments 190-196, wherein L₂ and L₃ are internucleoside linkages having formula VIII.

Embodiment 199. The oligomeric compound of any of embodiments 190-196, wherein L₃ and L₄ are internucleoside linkages having formula VIII.

Embodiment 200. The oligomeric compound of any of embodiments 190-196, wherein L₁, L₂, and L₃ are internucleoside linkages having formula VIII.

Embodiment 201. The oligomeric compound of any of embodiments 190-196, wherein L₂, L₃, and L₄,are internucleoside linkages having formula VIII.

Embodiment 202. The oligomeric compound of any of embodiments 190-196, wherein L₁, L₂ L₃, and L₄ are internucleoside linkages having formula VIII.

Embodiment 203. The oligomeric compound of any of embodiments 190-202, wherein R₁ is H.

Embodiment 204. The oligomeric compound of any of embodiments 190-202, wherein R₁ is a C₁-C₆ alkyl.

Embodiment 205. The oligomeric compound of embodiment 204, wherein R₁ is methyl.

Embodiment 206. The oligomeric compound of any of embodiments 190-202, wherein R₁ is a substituted C₁-C₆ alkyl; Embodiment 207. The oligomeric compound of any of embodiments 190-206, wherein T is SO₂R₂.

Embodiment 208. The oligomeric compound of embodiment 207, wherein R₂ is an aryl.

Embodiment 209. The oligomeric compound of embodiment 207, wherein R₂ is a substituted aryl.

Embodiment 210. The oligomeric compound of embodiment 207, wherein R₂ is a heterocycle.

Embodiment 211. The oligomeric compound of embodiment 207, wherein R₂ is a substituted heterocycle.

Embodiment 212. The oligomeric compound of embodiment 207, wherein R₂ is an aromatic heterocycle.

Embodiment 213. The oligomeric compound of embodiment 207, wherein R₂ is a substituted aromatic heterocycle.

Embodiment 214. The oligomeric compound of embodiment 207, wherein R₂ is a diazole.

Embodiment 215. The oligomeric compound of embodiment 207, wherein R₂ is a substituted diazole.

Embodiment 216. The oligomeric compound of embodiment 207, wherein R₂ is an amine.

Embodiment 217. The oligomeric compound of embodiment 207, wherein R₂ is a substituted amine.

Embodiment 218. The oligomeric compound of embodiment 207, wherein R₂ is a C₁-C₆ alkoxy.

Embodiment 219. The oligomeric compound of embodiment 207, wherein R₂ is C₁-C₆ alkyl.

Embodiment 220. The oligomeric compound of embodiment 207, wherein R₂ is substituted C₁-C₆ alkyl.

Embodiment 221. The oligomeric compound of embodiment 207, wherein T is:

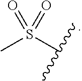 [SMILES: *S(C)(=O)=O]

Embodiment 222. The oligomeric compound of embodiment 207, wherein T is:

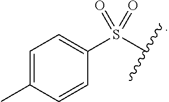 [SMILES: *S(=O)(=O)c1ccc(C)cc1]

Embodiment 223. The oligomeric compound of embodiment 207, wherein T is:

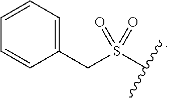 [SMILES: *S(=O)(=O)Cc1ccccc1]

Embodiment 224. The oligomeric compound of embodiment 207, wherein T is:

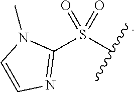 [SMILES: *S(=O)(=O)c1nccn1C]

Embodiment 225. The oligomeric compound of embodiment 207, wherein T is:

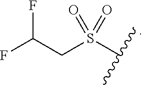 [SMILES: *S(=O)(=O)CC(F)F]

Embodiment 226. The oligomeric compound of embodiment 207, wherein T is:

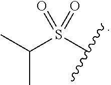 [SMILES: *S(=O)(=O)C(C)C]

Embodiment 227. The oligomeric compound of embodiment 207, wherein T is:

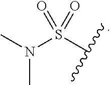 [SMILES: *S(=O)(=O)N(C)C]

Embodiment 228. The oligomeric compound of embodiment 207, wherein T is:

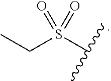 [SMILES: *S(=O)(=O)CC]

Embodiment 229. The oligomeric compound of embodiment 207, wherein T is:

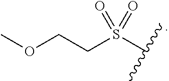 [SMILES: *S(=O)(=O)CCOC]

Embodiment 230. The oligomeric compound of any of embodiments 190-206, wherein T is C(═O)R₃.

Embodiment 231. The oligomeric compound of embodiment 230, wherein R₃ is an aryl.

Embodiment 232. The oligomeric compound of embodiment 230, wherein R₃ is a substituted aryl.

Embodiment 233. The oligomeric compound of embodiment 230, wherein R₃ is CH₃.

Embodiment 234. The oligomeric compound of embodiment 230, wherein R₃ is N(CH₃)2.

Embodiment 235. The oligomeric compound of embodiment 230, wherein R₃ is OCH₃.

Embodiment 236. The oligomeric compound of embodiment 230, wherein T is:

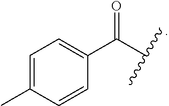 [SMILES: *C(=O)c1ccc(C)cc1]

Embodiment 237. The oligomeric compound of embodiment 230, wherein T is:

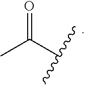 [SMILES: *C(C)=O]

Embodiment 238. The oligomeric compound of embodiment 230, wherein T is:

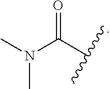 [SMILES: *C(=O)N(C)C]

Embodiment 239. The oligomeric compound of embodiment 230, wherein T is:

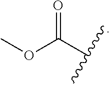 [SMILES: *C(=O)OC]

Embodiment 240. The oligomeric compound of any of embodiments 190-206, wherein T is P(═O)R₄R₅

Embodiment 241. The oligomeric compound of embodiment 240, wherein R₄ is OCH₃.

Embodiment 242. The oligomeric compound of embodiment 240, wherein R₄ is OH.

Embodiment 243. The oligomeric compound of embodiment 240, wherein R₄ is C₁-C₆ alkyl.

Embodiment 244. The oligomeric compound of embodiment 240, wherein R₄ is substituted C₁-C₆ alkyl.

Embodiment 245. The oligomeric compound of any of embodiments 240-242, wherein R₅ is OCH₃.

Embodiment 246. The oligomeric compound of any of embodiments 240-242, wherein R₅ is OH.

Embodiment 247. The oligomeric compound of any of embodiments 240-242, wherein R₅ is C₁-C₆ alkyl.

Embodiment 248. The oligomeric compound of any of embodiments 240-242, wherein R₅ is substituted C₁-C₆ alkyl.

Embodiment 249. The oligomeric compound of embodiment 240, wherein T is:

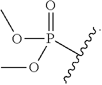 [SMILES: *P(=O)(OC)OC]

Embodiment 250. The oligomeric compound of embodiment 240, wherein T is:

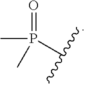 [SMILES: *P(C)(C)=O]

Embodiment 251. The oligomeric compound of any of embodiments 190-250, wherein at least one internucleoside linking group of the modified oligonucleotide is not a linking group of Formula VIII.

Embodiment 252. The oligomeric compound of any of embodiments 190-250, wherein exactly two internucleoside linking groups of the modified oligonucleotide are internucleoside linking groups of any of Formula VIII.

Embodiment 253. The oligomeric compound of any of embodiments 190-250, wherein exactly three internucleoside linking groups of the modified oligonucleotide are internucleoside linking groups of any of Formula VIII.

Embodiment 254. The oligomeric compound of any of embodiments 190-250, wherein exactly four internucleoside linking groups of the modified oligonucleotide are internucleoside linking groups of any of Formula VIII.

Embodiment 255. The oligomeric compound of any of embodiment 190-250 having at least two linking groups of Formula VIII, wherein at least two of the linking groups of Formula VIII are the same as one another.

Embodiment 256. The oligomeric compound of any of embodiments 190-255, wherein each internucleoside linking group of the modified oligonucleotide that is not an internucleoside linking group of Formula VIII is either a phosphodiester internucleoside linking group or a phosphorothioate internucleoside linking group.

Embodiment 257. The oligomeric compound of any of embodiments 190-256, wherein the 5′-region consists of 2-5 linked nucleosides.

Embodiment 258. The oligomeric compound of embodiment 257, wherein the 5′-region consists of 3 linked nucleosides.

Embodiment 259. The oligomeric compound of embodiment 257, wherein the 5′-region consists of 5 linked nucleosides.

Embodiment 260. The oligomeric compound of any of embodiments 190-259 wherein each nucleoside of the 5′-region is a modified nucleoside.

Embodiment 261. The oligomeric compound of any of embodiments 190-259, wherein each nucleoside of the 5′-region is a modified nucleoside comprising a modified sugar.

Embodiment 262. The oligomeric compound of any of embodiments 190-261, wherein each nucleoside of the 5′-region comprises a 2′-substituted furanosyl sugar moiety.

Embodiment 263. The oligomeric compound of any of embodiments 190-261, wherein at least one nucleoside of the 5′-region comprises a bicyclic furanosyl sugar moiety.

Embodiment 264. The oligomeric compound of any of embodiments 190-261, wherein each nucleoside of the 5′-region comprises a bicyclic furanosyl sugar moiety.

Embodiment 265. The oligomeric compound of embodiment 263, wherein each bicyclic sugar moiety of the 5′-region is selected from among cEt, LNA, and ENA.

Embodiment 266. The oligomeric compound of embodiment 263, wherein each bicyclic sugar moiety of the 5′-region is a cEt sugar moiety.

Embodiment 267. The oligomeric compound of embodiment 262, wherein each 2′-substituted furanosyl sugar moiety of the 5′-region is a ribosyl sugar moiety and has a 2′-substituent selected from among 2′-MOE, 2′-OMe, and 2′-NMA.

Embodiment 268. The oligomeric compound of any of embodiments 190-267, wherein each nucleobase of the 5′-region is independently selected from among thymine, uracil, guanine, cytosine, 5-methylcytosine, and adenine.

Embodiment 269. The oligomeric compound of any of embodiments 190-268, wherein the 3′-region consists of 2-5 linked nucleosides.

Embodiment 270. The oligomeric compound of embodiment 269, wherein the 3′-region consists of 3 linked nucleosides.

Embodiment 271. The oligomeric compound of embodiment 269, wherein the 3′-region consists of 5 linked nucleosides.

Embodiment 272. The oligomeric compound of any of embodiments 190-271 wherein each nucleoside of the 3′-region is a modified nucleoside.

Embodiment 273. The oligomeric compound of any of embodiments 190-272, wherein each nucleoside of the 3′-region is a modified nucleoside comprising a modified sugar.

Embodiment 274. The oligomeric compound of any of embodiments 190-273, wherein each nucleoside of the 3′-region comprises a 2′-substituted furanosyl sugar moiety.

Embodiment 275. The oligomeric compound of any of embodiments 190-273, wherein at least one nucleoside of the 3′-region comprises a bicyclic furanosyl sugar moiety.

Embodiment 276. The oligomeric compound of any of embodiments 190-273, wherein each nucleoside of the 3′-region comprises a bicyclic furanosyl sugar moiety.

Embodiment 277. The oligomeric compound of embodiment 276, wherein each bicyclic sugar moiety of the 3′-region is selected from among cEt, LNA, and ENA.

Embodiment 278. The oligomeric compound of embodiment 276, wherein each bicyclic sugar moiety of the 3′-region is a cEt sugar moiety.

Embodiment 279. The oligomeric compound of embodiment 274, wherein each wherein each 2′-substituted furanosyl sugar moiety of the 5′-region is a ribosyl sugar moiety and has a 2′-substituent selected from among 2′-MOE, 2′-OMe, and 2′-NMA.

Embodiment 280. The oligomeric compound of any of embodiments 190-279, wherein each nucleobase of the 5′-region is independently selected from among thymine, uracil, guanine, cytosine, 5-methylcytosine, and adenine.

Embodiment 281. The oligomeric compound of any of embodiments 1-80, wherein each nucleoside of the modified oligonucleotide is a modified nucleoside comprising a modified sugar moiety.

Embodiment 282. The oligomeric compound of embodiment 281, wherein each modified sugar moiety is independently selected from a bicyclic sugar moiety and a 2′-substituted furanosyl sugar moiety.

Embodiment 283. The oligomeric compound of embodiment 282, wherein the three 3′-most nucleosides comprise a bicyclic sugar moiety, and the remaining nucleosides comprise a 2′-substituted furanosyl sugar moiety.

Embodiment 284. The oligomeric compound of embodiment 282, wherein the four 3′-most nucleosides comprise a bicyclic sugar moiety, and the remaining nucleosides comprise a 2′-substituted furanosyl sugar moiety.

Embodiment 285. The oligomeric compound of embodiment 282, wherein the five 3′-most nucleosides comprise a bicyclic sugar moiety, and the remaining nucleosides comprise a 2′-substituted furanosyl sugar moiety.

Embodiment 286. The oligomeric compound of embodiment 282, wherein the six 3′-most nucleosides comprise a bicyclic sugar moiety, and the remaining nucleosides comprise a 2′-substituted furanosyl sugar moiety.

Embodiment 287. The oligomeric compound of any of embodiments 282-286, wherein each bicyclic sugar moiety is selected from among cEt, LNA, and ENA.

Embodiment 288. The oligomeric compound of embodiment 287, wherein the bicyclic sugar moiety is cEt Embodiment 289. The oligomeric compound of any of embodiments 282-289, wherein the 2′-substituted furanosyl sugar moiety is selected from 2′-OMe, 2′-MOE, and 2′-F.

Embodiment 290. The oligomeric compound of any of embodiments 281-289, wherein at least one of the first 10 internucleoside linking groups from the 5′-end of the modified oligonucleotide is an internucleoside linking group of any of embodiments 1-50.

Embodiment 291. The oligomeric compound of embodiment 289, wherein at least 2 of the first 10 internucleoside linking groups from the 5′-end of the modified oligonucleotide are internucleoside linking groups of any of embodiments 1-50.

Embodiment 292. The oligomeric compound of embodiment 289, wherein at least 3 of the first 10 internucleoside linking groups from the 5′-end of the modified oligonucleotide are internucleoside linking groups of any of embodiments 1-50.

Embodiment 293. The oligomeric compound of embodiment 289, wherein at least 4 of the first 10 internucleoside linking groups from the 5′-end of the modified oligonucleotide are internucleoside linking groups of any of embodiments 1-50.

Embodiment 294. The oligomeric compound of embodiment 289, wherein at least 5 of the first 10 internucleoside linking groups from the 5′-end of the modified oligonucleotide are internucleoside linking groups of any of embodiments 1-50.

Embodiment 295. The oligomeric compound of embodiment 289, wherein at least 6 of the first 10 internucleoside linking groups from the 5′-end of the modified oligonucleotide are internucleoside linking groups of any of embodiments 1-50.

Embodiment 296. The oligomeric compound of embodiment 289, wherein the first 2 internucleoside linking groups from the 5′-end of the modified oligonucleotide are internucleoside linking groups of any of embodiments 1-50.

Embodiment 297. The oligomeric compound of embodiment 289, wherein the first 3 internucleoside linking groups from the 5′-end of the modified oligonucleotide are internucleoside linking groups of any of embodiments 1-50.

Embodiment 298. The oligomeric compound of embodiment 289, wherein the first 4 internucleoside linking groups from the 5′-end of the modified oligonucleotide are internucleoside linking groups of any of embodiments 1-50.

Embodiment 299. The oligomeric compound of embodiment 289, wherein the first 5 internucleoside linking groups from the 5′-end of the modified oligonucleotide are internucleoside linking groups of any of embodiments 1-50.

Embodiment 300. The oligomeric compound of embodiment 289, wherein the first 6 internucleoside linking groups from the 5′-end of the modified oligonucleotide are internucleoside linking groups of any of embodiments 1-50.

Embodiment 301. A method of increasing translation of a target protein in a cell, comprising contacting the cell with an oligomeric compound of any of embodiments 281-300.

Embodiment 302. The method of embodiment 301, wherein the target protein is encoded by a target nucleic acid comprising at least one translation suppression element and wherein the modified oligonucleotide is complementary to a target site within a translation suppression element region of the target nucleic acid.

Embodiment 303. The method of embodiment 302, wherein the translation suppression element region comprises at least one stem-loop structure.

Embodiment 304. A pharmaceutical composition comprising the oligomeric compound of any of embodiments 190-300 and a pharmaceutically acceptable carrier or diluent.

Embodiment 305. A method comprising contacting a cell with the oligomeric compound or pharmaceutical composition of any of embodiments 190-300.

Embodiment 306. A method of modulating the amount or activity of a target nucleic acid in a cell, comprising contacting the cell with the oligomeric compound or pharmaceutical composition of any of embodiments 190-300, and thereby modulating the amount or activity of the target nucleic acid.

Embodiment 307. The method of embodiment 306, wherein the amount or activity of a target nucleic acid is reduced.

Embodiment 308. The method of embodiment 306, wherein the amount or activity of a target nucleic acid is increased.

Embodiment 309. Use of the oligomeric compound or composition of any of embodiments 190-300 for treatment of a disease or condition.

Embodiment 310. Use of the oligomeric compound or composition of any of embodiments 190-300 for a preparation of a medicament for treatment of a disease or condition.

Embodiment 311. An antisense agent comprising a modified oligonucleotide consisting of linked nucleosides linked through internucleoside linking groups, wherein at least one of the internucleoside linking groups has Formula XVII:

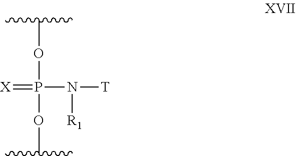

wherein independently for each internucleoside linking group of the modified oligonucleotide having Formula XVII:
X is selected from O or S;
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle,
a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group; and
R₅ is selected from OCH₃, OH, C₁-C₆ alkyl, and substituted C₁-C₆ alkyl.

Embodiment 312. An antisense agent comprising a modified oligonucleotide consisting of linked nucleosides linked through internucleoside linking groups, wherein at least one of the internucleoside linking groups has Formula XVII:

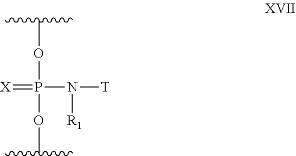

wherein independently for each internucleoside linking group of the modified oligonucleotide having Formula XVII:
X is selected from O or S;
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group; and
R₅ is selected from OCH₃, OH, C₁-C₆ alkyl, and substituted C₁-C₆ alkyl;
Provided that if X is O and that if R₁ is H, then T is not:

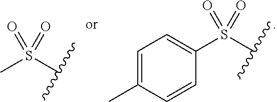

Embodiment 313. The modified oligonucleotide of embodiment 311 or 312, wherein for at least one internucleoside linking group of Formula XVII, X is O.

Embodiment 314. The modified oligonucleotide of embodiment 311 or 312, wherein for at least one internucleoside linking group of Formula XVII, X is S.

Embodiment 315. The modified oligonucleotide of embodiment 311 or 312, wherein for at least one internucleoside linking group of Formula XVII, R₁ is H.

Embodiment 316. The modified oligonucleotide of embodiment 311 or 312, wherein for at least one internucleoside linking group of Formula XVII, R₁ is a C₁-C₆ alkyl, C₁-C₆ alkenyl, or C₁-C₆ alkynyl.

Embodiment 317. The modified oligonucleotide of embodiment 6, wherein R₁ is methyl.

Embodiment 318. The modified oligonucleotide of embodiment 311 or 312, wherein for at least one internucleoside linking group of Formula XVII, R₁ is a substituted C₁-C₆ alkyl.

Embodiment 319. The modified oligonucleotide of any of embodiments 311-318, wherein for at least one internucleoside linking group of Formula XVII, T comprises a conjugate group.

Embodiment 320. The modified oligonucleotide of embodiment 319, wherein the conjugate group comprises a carbohydrate or carbohydrate cluster.

Embodiment 321. The modified oligonucleotide of embodiment 319 or 320, wherein the conjugate group comprises at least one GalNAc.

Embodiment 322. The modified oligonucleotide of embodiment 319, wherein the conjugate group comprises a C₁₀-C₂₀ alkyl chain.

Embodiment 323. The modified oligonucleotide of embodiment 322, wherein the conjugate group comprises C₁₆ alkyl.

Embodiment 324. The modified oligonucleotide of any of embodiments 311-318, wherein for at least one internucleoside linking group of formula XVII, T does not comprise a conjugate group.

Embodiment 325. The modified oligonucleotide of any of embodiments 311-324, wherein for at least one internucleoside linking group of Formula XVII, T is SO₂R₂.

Embodiment 326. The modified oligonucleotide of embodiment 325, wherein R₂ is an aryl.

Embodiment 327. The modified oligonucleotide of embodiment 325, wherein R₂ is a substituted aryl.

Embodiment 328. The modified oligonucleotide of embodiment 325, wherein R₂ is a heterocycle.

Embodiment 329. The modified oligonucleotide of embodiment 325, wherein R₂ is a substituted heterocycle.

Embodiment 330. The modified oligonucleotide of embodiment 325, wherein R₂ is an aromatic heterocycle.

Embodiment 331. The modified oligonucleotide of embodiment 325, wherein R₂ is a substituted aromatic heterocycle.

Embodiment 332. The modified oligonucleotide of embodiment 325, wherein R₂ is a diazole.

Embodiment 333. The modified oligonucleotide of embodiment 325, wherein R₂ is a substituted diazole.

Embodiment 334. The modified oligonucleotide of embodiment 325, wherein R₂ is an amine.

Embodiment 335. The modified oligonucleotide of embodiment 325, wherein R₂ is a substituted amine.

Embodiment 336. The modified oligonucleotide of embodiment 325, wherein R₂ is a C₁-C₆ alkoxy, or C₁-C₆ alkenyl, C₁-C₆ alkynyl.

Embodiment 337. The modified oligonucleotide of embodiment 325, wherein R₂ is C₁-C₂₀, C₁-C₆, C₂-C₂0, C₂-C₆, or C₁₀-C₂₀ alkyl.

Embodiment 338. The modified oligonucleotide of embodiment 325, wherein R₂ is substituted C₁-C₂₀, C₁-C₆, C₂-C₂₀, C₂-C₆, or C₁₀-C₂₀ alkyl.

Embodiment 339. The modified oligonucleotide of embodiment 325, wherein R₂ comprises a carbohydrate or carbohydrate cluster.

Embodiment 340. The modified oligonucleotide of embodiment 325, wherein R₂ comprises at least one GalNAc.

Embodiment 341. The modified oligonucleotide of embodiment 325, wherein T is:

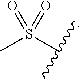 [SMILES: *S(C)(=O)=O]

Embodiment 342. The modified oligonucleotide of embodiment 325, wherein T is:

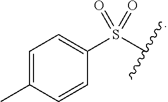 [SMILES: *S(=O)(=O)c1ccc(C)cc1]

Embodiment 343. The modified oligonucleotide of embodiment 325, wherein T is:

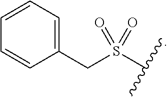 [SMILES: *S(=O)(=O)Cc1ccccc1]

Embodiment 344. The modified oligonucleotide of embodiment 325, wherein T is:

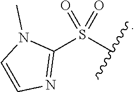 [SMILES: *S(=O)(=O)c1nccn1C]

Embodiment 345. The modified oligonucleotide of embodiment 325, wherein T is:

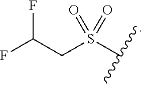 [SMILES: *S(=O)(=O)CC(F)F]

Embodiment 346. The modified oligonucleotide of embodiment 325, wherein T is:

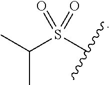 [SMILES: *S(=O)(=O)C(C)C]

Embodiment 347. The modified oligonucleotide of embodiment 325, wherein T is:

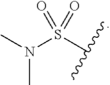 [SMILES: *S(=O)(=O)N(C)C]

Embodiment 348. The modified oligonucleotide of embodiment 325, wherein T is:

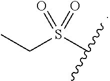 [SMILES: *S(=O)(=O)CC]

Embodiment 349. The modified oligonucleotide of embodiment 325, wherein T is:

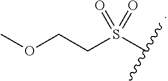 [SMILES: *S(=O)(=O)CCOC]

Embodiment 350. The modified oligonucleotide of embodiment 325, wherein T is:

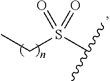

wherein n is from 2 to 20.

Embodiment 351. The modified oligonucleotide of embodiment 350, wherein n is 15.

Embodiment 352. The modified oligonucleotide of any of embodiments 311-324, wherein for at least one internucleoside linking group of Formula XVII, T is C(═O)R₃.

Embodiment 353. The modified oligonucleotide of embodiment 352, wherein R₃ is an aryl.

Embodiment 354. The modified oligonucleotide of embodiment 352, wherein R₃ is a substituted aryl.

Embodiment 355. The modified oligonucleotide of embodiment 352, wherein R₃ is CH₃.

Embodiment 356. The modified oligonucleotide of embodiment 352, wherein R₃ is N(CH₃)₂.

Embodiment 357. The modified oligonucleotide of embodiment 352, wherein R₃ is OCH₃.

Embodiment 358. The modified oligonucleotide of embodiment 352, wherein R₃ is a C₁-C₆ alkoxy.

Embodiment 359. The modified oligonucleotide of embodiment 352, wherein R₃ is C₁-C₂₀, C₁-C₆, C₂-C₂₀, C₂-C₆, or C₁₀-C₂₀ alkyl.

Embodiment 360. The modified oligonucleotide of embodiment 352, wherein R₃ is substituted C₁-C₂₀, C₁-C₆, C₂-C₂₀, C₂-C₆, or C₁₀-C₂₀ alkyl.

Embodiment 361. The modified oligonucleotide of embodiment 352, wherein R₃ comprises a carbohydrate or carbohydrate cluster.

Embodiment 362. The modified oligonucleotide of embodiment 352, wherein R₂₃ comprises at least one GalNAc.

Embodiment 363. The modified oligonucleotide of embodiment 352, wherein T is:

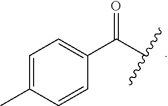 [SMILES: *C(=O)c1ccc(C)cc1]

Embodiment 364. The modified oligonucleotide of embodiment 352, wherein T is:

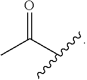 [SMILES: *C(C)=O]

Embodiment 365. The modified oligonucleotide of embodiment 352, wherein T is:

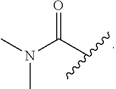 [SMILES: *C(=O)N(C)C]

Embodiment 366. The modified oligonucleotide of embodiment 352, wherein T is:

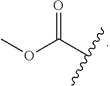 [SMILES: *C(=O)OC]

Embodiment 367. The modified oligonucleotide of embodiment 352, wherein T is:

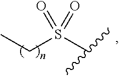

wherein n is from 2 to 20.

Embodiment 368. The modified oligonucleotide of embodiment 367, wherein n is 15.

Embodiment 369. The modified oligonucleotide of any of embodiments 1-14, wherein for at least one internucleoside linking group of Formula XVII, T is P(═O)R₄R₅.

Embodiment 370. The modified oligonucleotide of embodiment 369, wherein R₄ is OCH₃.

Embodiment 371. The modified oligonucleotide of embodiment 369, wherein R₄ is OH.

Embodiment 372. The modified oligonucleotide of embodiment 369, wherein R₄ is C₁-C₆ alkyl.

Embodiment 373. The modified oligonucleotide of embodiment 369, wherein R₄ is substituted C₁-C₆ alkyl.

Embodiment 374. The modified oligonucleotide of embodiment 369, wherein R₄ is C₁-C₂₀, C₁-C₆, C₂-C₂₀, C₂-C₆, or C₁₀-C₂₀ alkyl.

Embodiment 375. The modified oligonucleotide of embodiment 369, wherein R₄ is substituted C₁-C₂₀, C₁-C₆, C₂-C₂₀, C₂-C₆, or C₁₀-C₂₀ alkyl.

Embodiment 376. The modified oligonucleotide of embodiment 369, wherein R₄ comprises a carbohydrate or carbohydrate cluster.

Embodiment 377. The modified oligonucleotide of embodiment 369, wherein R₄ comprises at least one GalNAc.

Embodiment 378. The modified oligonucleotide of any of embodiments 369-67, wherein R₅ is OCH₃.

Embodiment 379. The modified oligonucleotide of any of embodiments 369-67, wherein R₅ is OH

Embodiment 380. The modified oligonucleotide of any of embodiments 369-67, wherein R₅ is C₁-C₆ alkyl.

Embodiment 381. The modified oligonucleotide of any of embodiments 369-67, wherein R₅ is substituted C₁-C₆ alkyl.

Embodiment 382. The modified oligonucleotide of embodiment 369, wherein T is:

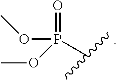 [SMILES: *P(=O)(OC)OC]

Embodiment 383. The modified oligonucleotide of embodiment 369, wherein T is:

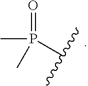 [SMILES: *P(C)(C)=O]

Embodiment 384. The modified oligonucleotide of embodiment 369, wherein T is:

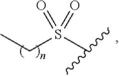

wherein n is from 2 to 20.

Embodiment 385. The modified oligonucleotide of embodiment 384, wherein n is 15.

Embodiment 386. The modified oligonucleotide of any of embodiments 311-385, wherein at least one internucleoside linking group of the modified oligonucleotide is not a linking group of Formula XVII.

Embodiment 387. The modified oligonucleotide of any of embodiments 311-385, wherein exactly one internucleoside linking group of the modified oligonucleotide is an internucleoside linking group of Formula XVII.

Embodiment 388. The modified oligonucleotide of any of embodiments 311-385, wherein exactly two internucleoside linking groups of the modified oligonucleotide are internucleoside linking groups of Formula XVII.

Embodiment 389. The modified oligonucleotide of any of embodiments 311-385, wherein exactly three internucleoside linking groups of the modified oligonucleotide are internucleoside linking groups of Formula XVII.

Embodiment 390. The modified oligonucleotide of any of embodiments 311-385, wherein exactly four internucleoside linking groups of the modified oligonucleotide are internucleoside linking groups of Formula XVII.

Embodiment 391. The modified oligonucleotide of any of embodiments 311-385, wherein exactly five internucleoside linking groups of the modified oligonucleotide are internucleoside linking groups of Formula XVII.

Embodiment 392. The modified oligonucleotide of any of embodiments 311-385, wherein at least six internucleoside linking groups of the modified oligonucleotide are internucleoside linking groups of Formula XVII.

Embodiment 393. The modified oligonucleotide of any of embodiment 311-386 or 388-392 having at least two linking groups of Formula XVII, wherein at least two of the linking groups of Formula XVII are the same as one another.

Embodiment 394. The modified oligonucleotide of any of embodiments 311-393, wherein each internucleoside linking group of the modified oligonucleotide that is not an internucleoside linking group of Formula XVII is either a phosphodiester internucleoside linking group or a phosphorothioate internucleoside linking group.

Embodiment 395. The modified oligonucleotide of any of embodiments 311-386 or 393-394, wherein each internucleoside linking group of the modified oligonucleotide is an internucleoside linking group of Formula XVII.

Embodiment 396. An antisense agent comprising a modified oligonucleotide, wherein at least one region of the modified oligonucleotide has Structure A:

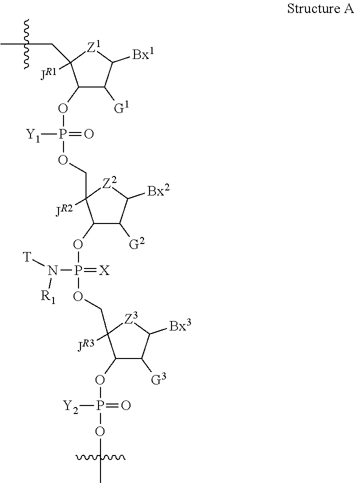

wherein:
each Bx is a heterocyclic base moiety;
X is selected from O or S;
each of Y₁ and Y₂ is independently selected from OH or SH;
each of Z¹, Z², and Z³ are independently selected from —(CH₂)ₚ-X^{Z}—(CH₂)_{q}—, wherein p is 0 or 1, q is 0 or 1, and X^{Z} is O, S, or N(E₁);
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle,
a substituted aromatic heterocycle, a diazole, a substituted diazole, a C₁-C₆ alkoxy, C₁-C₆ alkyl, C₁-C₆ alkenyl,
C₁-C₆ alkynyl, substituted C₁-C₆ alkyl, substituted C₁-C₆ alkenyl, substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group;
R₅ is selected from OCH₃, OH, C₁-C₆ alkyl, and substituted C₁-C₆ alkyl;
either J^{R1} and G¹ form a J^{R1} to G¹ bridge, or J^{R1} is H and G¹ is selected from H, OH, halogen or O—[C(R₆)(R₇)]ₙ—[(C═O)ₘ—X^{G}]ⱼ—R₈;
either J^{R2} and G² form a J^{R2} and G² bridge, or J^{R2} is H and G² is selected from H, OH, halogen or O—[C(R₆)(R₇)]n-[(C═O)ₘ—X^{G}]ⱼ—R₈;
either J^{R3} and G³ form a J^{R3} and G³ bridge, or J^{R3} is H and G³ is selected from H, OH, halogen or O—[C(R₆)(R₇)]ₙ—[(C═O)ₘ—X^{G}]ⱼ—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

Embodiment 397. An antisense agent comprising a modified olignucleotide, wherein at least one region of the modified oligonucleotide has Structure B:

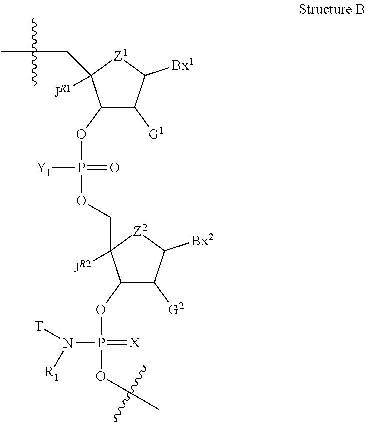

wherein:
   each Bx is a heterocyclic base moiety;
X is selected from O or S;
each of Y₁ and Y₂ is independently selected from OH or SH;
each of Z¹ and Z² are independently selected from —(CH₂)ₚ—X^{Z}—(CH₂)_{q}, wherein p is 0 or 1, q is 0 or 1, and X^{Z} is O, S, or N(E₁);
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl, substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group;
R₅ is selected from OCH₃, OH, C₁-C₆ alkyl, and substituted C₁-C₆ alkyl;
either J^{R1} and G¹ form a J^{R1} to G¹ bridge, or J^{R1} is H and G¹ is selected from H, OH, halogen or O—[C(R₆)(R₇)]ₙ—[(C═O)ₘ—X^{G}]ⱼ—R₈;
either J^{R2} and G² form a J^{R2} and G² bridge, or J^{R2} is H and G² is selected from H, OH, halogen or O—[C(R₆)(R₇)]ₙ—[(C═O)ₘ—X^{G}]ⱼ—R₈;
wherein each J^{R} to G bridge has a formula independently selected from —CH(CH₃)—O— or —(CH₂)k-O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

Embodiment 398. An antisense agent comprising a modified olignucleotide, wherein at least one region of the modified oligonucleotide has Structure C:

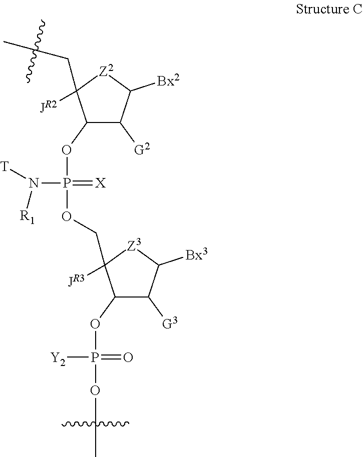

wherein:
each Bx is a heterocyclic base moiety;
X is selected from O or S;
each of Y₁ and Y₂ is independently selected from OH or SH;
each of Z² and Z³ are independently selected from —(CH₂)ₚ—X^{Z}—(CH₂)_{q}—, wherein p is 0 or 1, q is 0 or 1, and X^{Z} is O, S, or N(E₁);
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl, substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group;
R₅ is selected from OCH₃, OH, C₁-C₆ alkyl, and substituted C₁-C₆ alkyl;
   either J^{R2} and G² form a J^{R2} and G² bridge, or J^{R2} is H and G² is selected from H, OH, halogen or O—[C(R₆)(R₇)]ₙ—[(C═O)ₘ—X^{G}]ⱼ—R₈;
either J^{R3} and G³ form a J^{R3} and G³ bridge, or J^{R3} is H and G³ is selected from H, OH, halogen or O—[C(R₆)(R₇)]ₙ—[(C═O)ₘ—X^{G}]ⱼ—R₈;
wherein each J^{R} to G bridge has a formula independently selected from —CH(CH₃)—O— or —(CH₂)k-O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

Embodiment 399. An antisense agent comprising a modified olignucleotide, wherein at least one region of the modified oligonucleotide has Structure D:

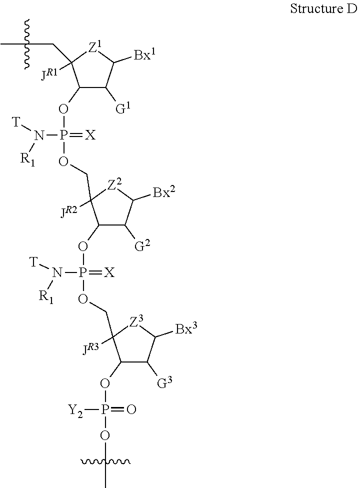

wherein:
each Bx is a heterocyclic base moiety;
X is selected from O or S;
each of Y₁ and Y₂ is independently selected from OH or SH;
each of Z² and Z³ are independently selected from —(CH₂)ₚ—X^{Z}—(CH₂)_{q}—, wherein p is 0 or 1, q is 0 or 1, and X^{Z} is O, S, or N(E₁);
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl, substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group;
R₅ is selected from OCH₃, OH, C₁-C₆ alkyl, and substituted C₁-C₆ alkyl;
either J^{R1} and G¹ form a J^{R1} to G¹ bridge, or J^{R1} is H and G¹ is selected from H, OH, halogen or O—[C(R₆)(R₇)]ⱼ—[(C═O)ₘ—X^{G}]—R₈;
either J^{R2} and G² form a J^{R2} and G² bridge, or J^{R2} is H and G² is selected from H, OH, halogen or O—[C(R₆)(R₇)]ₙ—[(C═O)ₘ—X^{G}]—R₈;
either J^{R3} and G³ form a J^{R3} and G³ bridge, or J^{R3} is H and G³ is selected from H, OH, halogen or O—[C(R₆)(R₇)]ₙ—[(C═O)ₘ—X^{G}]—R₈;
wherein each J^{R} to G bridge has a formula independently selected from —CH(CH₃)—O— or —(CH₂)k-O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂); Q₂ is O, S or NJ₃;
each J₁, J₂ and J₃ is, independently, H or C₁-C₆ alkyl.

Embodiment 400. An antisense agent comprising a modified olignucleotide, wherein at least one region of the modified oligonucleotide has Structure E:

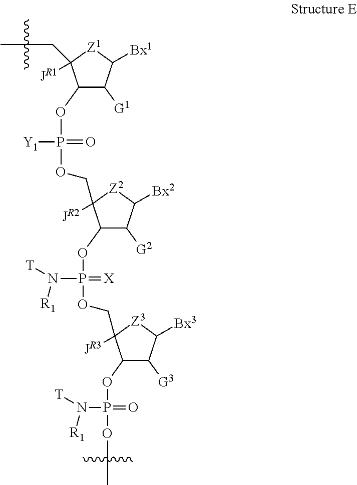

wherein:
each Bx is a heterocyclic base moiety;
X is selected from O or S;
each of Y₁ and Y₂ is independently selected from OH or SH;
each of Z² and Z³ are independently selected from —(CH₂)ₚ—X^{Z}—(CH₂)_{q}—, wherein p is 0 or 1, q is 0 or 1, and X^{Z} is O, S, or N(E₁);
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl, substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group;
R₅ is selected from OCH₃, OH, C₁-C₆ alkyl, and substituted C₁-C₆ alkyl;
either J^{R1} and G¹ form a J^{R1} to G¹ bridge, or J^{R1} is H and G¹ is selected from H, OH, halogen or O—[C(R₆)(R₇)]—[(C═O)ₘ—X^{G}]ⱼ—R₈;
either J^{R2} and G² form a J^{R2} and G² bridge, or J^{R2} is H and G² is selected from H, OH, halogen or O—[C(R₆)(R₇)]ₙ—[(C═O)ₘ—X^{G}]ⱼ—R₈;
either J^{R3} and G³ form a J^{R3} and G³ bridge, or J^{R3} is H and G³ is selected from H, OH, halogen or O—[C(R₆)(R₇)]ₙ—[(C═O)ₘ—X^{G}]ⱼ—R₈;
wherein each J^{R} to G bridge has a formula independently selected from —CH(CH₃)—O— or —(CH₂)k-O—, wherein k is from 1 to 3;
each R₆ and R₇ is, independently, H, halogen, C₁-C₆ alkyl or substituted C₁-C₆ alkyl;
each X^{G} is O, S or N(E₁); R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂); Q₂ is O, S or NJ₃;
each J₁, J₂ and J₃ is, independently, H or C₁-C₆ alkyl.

Embodiment 401. The modified oligonucleotide of any of embodiments 396-400, wherein each Z is O.

Embodiment 402. The modified oligonucleotide of any of embodiments 396-401, wherein at least one G is selected from H, OH, halogen, C₁-C₆ alkoxy, —O(CH₂)₂OCH₃, or —OCH₂(C═O)NHCH₃.

Embodiment 403. The modified oligonucleotide of any of embodiments 396-401, wherein each G is selected from H, OH, halogen, C₁-C₆ alkoxy, —O(CH₂)₂OCH₃, or —OCH₂(C═O)NHCH₃.

Embodiment 404. The modified oligonucleotide of any of embodiments 396-402, wherein at least one J^{R} forms a bridge with at least one G, wherein said J^{R} to G bridge has a formula selected from 4CH(CH₃)—O— or —(CH₂)ₖ—O′, wherein k is from 1 to 3.

Embodiment 405. The modified oligonucleotide of any of embodiments 396-402, wherein each J^{R} and G form a bridge, wherein said J^{R} to G bridge has a formula selected from —CH(CH₃)—O— or —(CH₂)ₖ—O—, wherein k is from 1 to 3.

Embodiment 406. The modified oligonucleotide of any of embodiments 404 or 405, wherein at least one Z is 0 and the corresponding J^{R} to G bridge has a formula (CH₂)ₖ—O—, wherein k is 1.

Embodiment 407. The modified oligonucleotide of any of embodiments 396-406 wherein each nucleoside of structure A, B, C, D, or E is a stereo standard nucleoside.

Embodiment 408. The modified oligonucleotide of any of embodiments 396-406, wherein at least one nucleoside of structure A, B, C, D, or E is a stereo-non-standard nucleoside.

Embodiment 409. The modified oligonucleotide of any of embodiments 404-406 or 408, wherein at least one nucleoside having a J^{R} to G bridge is in the α-L-ribosyl configuration.

Embodiment 410. The modified oligonucleotide of any of embodiments 396-409, wherein the modified oligonucleotide comprises at least 2, at least 3, at least 4, at least 5, at least 6, at least 7, at least 8, at least 9, or at least 10 regions having structures A, B, C, D, or E.

Embodiment 411. The modified oligonucleotide of any of embodiments 396-410, wherein at least one region having structure A, B, C, D, or E is at the 5′ end of the modified oligonucleotide.

Embodiment 412. The modified oligonucleotide of any of embodiments 396-410, wherein at least one region having structure A, B, C, D, or E is at the 3′ end of the modified oligonucleotide.

Embodiment 413. The modified oligonucleotide of any of embodiments 396-410, wherein at least one region having structure A, B, C, D, or E is internal to the modified oligonucleotide.

Embodiment 414. An antisense agent, comprising a modified oligonucleotide consisting of 10-30 linked nucleosides, wherein a region of the modified oligonucleotide has the formula (N_{g1})_{L1}(N_{g2})_{L2}(N_{g3})_{L3}, wherein each N_{g} is a nucleoside and each L is an internucleoside linking group; wherein each of L₁, and L₂ is a phosphodiester internucleoside linking group, a phosphorothioate internucleoside linking group, or an internucleoside linking group of Formula XVII:

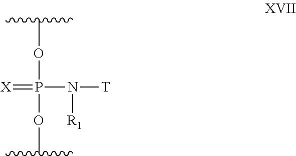

wherein L₃ is absent or is a phosphodiester internucleoside linking group, a phosphorothioate internucleoside linking group, or an internucleoside linking group of Formula XVII;
wherein at least one of L₁, L₂, and L₃ an internucleoside linking group of Formula XVII; and at least one of L₁, L₂, and L₃ is a phosphorothioate or a phosphodiester internucleoside linking group,
   wherein independently for each internucleoside linking group of the modified oligonucleotide having Formula XVII:
   X is selected from O or S;
   R₁ is selected from H, C₁-C₆ alkyl, and substituted C₁-C₆ alkyl; and
   T is selected from SO₂R₂, C(═O)R₃, and P(═O)R₄R₅, wherein:
   R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl, substituted C₁-C₆ alkynyl, and a conjugate group;
   R₃ is selected from an aryl, a substituted aryl, CH₃, N(CH₃)₂, OCH₃ and a conjugate;
   R₄ is selected from OCH₃, OH, C₁-C₆ alkyl, substituted C₁-C₆ alkyl and a conjugate; and
      R₅ is selected from OCH₃, OH, C₁-C₆ alkyl, and substituted C₁-C₆ alkyl.

Embodiment 415. The modified oligonucleotide of embodiment 414, wherein the modified oligonucleotide comprises at least 2, at least 3, at least 4, at least 5, at least 6, at least 7, at least 8, at least 9, or at least 10 regions having the formula (N_{g1})_{L1}(N_{g2})_{L2}(N_{g3})_{L3}.

Embodiment 416. The modified oligonucleotide of embodiment 414, wherein at least one region having the formula (N_{g1})_{L1}(N_{g2})_{L2}(N_{g3})_{L3} is at the 5′ end of the oligonucleotide

Embodiment 417. The modified oligonucleotide of embodiment 414, wherein at least one region having the formula (N_{g1})_{L1}(N_{g2})_{L2}(N_{g3})_{L3} is internal to the oligonucleotide.

Embodiment 418. The modified oligonucleotide of embodiment 414, wherein at least one region having the formula (N_{g1})_{L1}(N_{g2})_{L2}(N_{g3})_{L3} is at the 3′ end of the oligonucleotide.

Embodiment 419. The modified oligonucleotide of any of embodiments 311-418, wherein at least one nucleoside of the modified oligonucleotide is a modified nucleoside.

Embodiment 420. The modified oligonucleotide of embodiment 419, wherein at least one nucleoside of the modified oligonucleotide is a modified nucleoside selected from a bicyclic nucleoside and a non-bicyclic substituted nucleoside.

Embodiment 421. The modified oligonucleotide of any of embodiments 311-420, wherein at least one nucleoside of the modified oligonucleotide is selected from: a β-D-LNA nucleoside, an α-L-LNA nucleoside, an ENA nucleoside, a cEt nucleoside, a 2′-MOE nucleoside, a 2′-OMe nucleoside, a 2′-F nucleoside, a 2′-NMA nucleoside, a 5′-Me nucleoside, a DNA nucleoside, and an RNA nucleoside.

Embodiment 422. The modified oligonucleotide of any of embodiments 311-421, wherein each nucleoside of the modified oligonucleotide is selected from: a β-D-LNA nucleoside, an α-L-LNA nucleoside, an ENA nucleoside, a cEt nucleoside, a 2′-MOE nucleoside, a 2′-OMe nucleoside, a 2′-F nucleoside, a 2′-NMA nucleoside, a 5′-Me nucleoside, a DNA nucleoside, and an RNA nucleoside.

Embodiment 423. The modified oligonucleotide of any of embodiments 311-422, wherein at least one nucleoside of the modified oligonucleotide is a stereo-non-standard nucleoside.

Embodiment 424. The modified oligonucleotide of embodiment 423, wherein the internucleoside linking group linking at least one stereo-non-standard nucleoside to an adjacent nucleoside is an internucleoside linking group of Formula XVII.

Embodiment 425. The modified oligonucleotide of embodiment 423 or 424, wherein at least two nucleosides of the modified oligonucleotide are stereo-non-standard nucleosides.

Embodiment 426. The modified oligonucleotide of embodiment 425, wherein at least two stereo-non-standard nucleosides of the modified oligonucleotide are adjacent to one another.

Embodiment 427. The modified oligonucleotide of embodiment 426, wherein at least two stereo-non-standard nucleosides of the modified oligonucleotide are linked to one another with an internucleoside linking group of Formula XVII.

Embodiment 428. The modified oligonucleotide of any of embodiments 423-427, wherein at least one stereo-non-standard nucleoside of the modified oligonucleotide is a stereo-non-standard DNA nucleoside.

Embodiment 429. The modified oligonucleotide of embodiment 428 wherein the stereo-non-standard DNA nucleoside is selected from a stereo-non-standard DNA nucleoside having: Formula I, Formula II, Formula III, Formula IV, Formula V, Formula VI, and Formula VII.

Embodiment 430. The modified oligonucleotide of embodiment 429 wherein the stereo-non-standard DNA nucleoside is selected from a stereo-non-standard DNA nucleoside having: Formula V and Formula II.

Embodiment 431. The modified oligonucleotide of any of embodiments 423-427, wherein at least one stereo-non-standard nucleoside of the modified oligonucleotide is a substituted stereo-non-standard nucleoside or a stereo-non-standard RNA nucleoside.

Embodiment 432. The modified oligonucleotide of embodiment 431, wherein the 2′-substituent of the at least one substituted stereo-non-standard nucleoside of the modified oligonucleotide is selected from: 2′-MOE, 2′-OMe, 2′-F, or 2′-OH.

Embodiment 433. The modified oligonucleotide of any of embodiments 311-432, wherein the modified oligonucleotide consists of 12-30 linked nucleosides.

Embodiment 434. The modified oligonucleotide of any of embodiments 311-433, wherein the modified oligonucleotide consists of 16-24 linked nucleosides.

Embodiment 435. The modified oligonucleotide of any of embodiments 311-434, wherein the modified oligonucleotide consists of 18-22 linked nucleosides.

Embodiment 436. The modified oligonucleotide of any of embodiments 311-434, wherein the modified oligonucleotide consists of 16 linked nucleosides.

Embodiment 437. The modified oligonucleotide of any of embodiments 311-434, wherein the modified oligonucleotide consists of 17 linked nucleosides.

Embodiment 438. The modified oligonucleotide of any of embodiments 311-435, wherein the modified oligonucleotide consists of 18 linked nucleosides.

Embodiment 439. The modified oligonucleotide of any of embodiments 311-435, wherein the modified oligonucleotide consists of 19 linked nucleosides.

Embodiment 440. The modified oligonucleotide of any of embodiments 311-435, wherein the modified oligonucleotide consists of 20 linked nucleosides.

Embodiment 441. The modified oligonucleotide of any of embodiments 311-435, wherein the modified oligonucleotide consists of 21 linked nucleosides.

Embodiment 442. The modified oligonucleotide of any of embodiments 311-435, wherein the modified oligonucleotide consists of 22 linked nucleosides.

Embodiment 443. The modified oligonucleotide of any of embodiments 311-434, wherein the modified oligonucleotide consists of 23 linked nucleosides.

Embodiment 444. The modified oligonucleotide of any of embodiments 311-443, wherein at least one nucleoside of the modified oligonucleotide is selected from: a 2′-OMe nucleoside, a 2′-F nucleoside, and an RNA nucleoside.

Embodiment 445. The modified oligonucleotide of any of embodiments 311-444, wherein at least one nucleoside of the modified oligonucleotide is a 2′-OMe nucleoside, and at least one nucleoside of the modified oligonucleotide is an RNA nucleoside.

Embodiment 446. The modified oligonucleotide of any of embodiments 444-445, wherein the modified oligonucleotide has a region of alternating nucleoside types having the motif ABABA, wherein each A is a stereo-standard nucleoside of a first type and each B is a stereo-standard nucleoside of a second type, wherein the first type and the second type are different from one another.

Embodiment 447. The modified oligonucleotide of embodiment 446, wherein A and B are selected from 2′-F substituted nucleosides, 2′-OMe substituted nucleosides, and stereo-standard RNA nucleosides.

Embodiment 448. The modified oligonucleotide of any of embodiments 311-447, wherein the 5′-end of the modified oligonucleotide comprises a stabilized phosphate group.

Embodiment 449. The modified oligonucleotide of embodiment 448, wherein the stabilized phosphate group is a 5′-vinyl phosphonate or a 5′-cyclopropyl phosphonate.

Embodiment 450. An RNAi agent, comprising a modified oligonucleotide of any of embodiments 311-449.

Embodiment 451. The RNAi agent of embodiment 450, wherein the RNAi agent is a single-stranded RNAi agent comprising an RNAi antisense modified oligonucleotide, wherein the RNAi antisense modified oligonucleotide is a modified oligonucleotide of any of embodiments 311-449.

Embodiment 452. The RNAi agent of embodiment 450, wherein the RNAi agent is an oligomeric duplex comprising an RNAi antisense modified oligonucleotide and an RNAi sense modified oligonucleotide, wherein the RNAi antisense modified oligonucleotide and/or the RNAi sense modified oligonucleotide is a modified oligonucleotide of any of embodiments 311-449.

Embodiment 453. The RNAi agent of embodiment 451 or 452, wherein at least one internucleoside linking group of the RNAi antisense modified oligonucleotide is an internucleoside linking group of Formula XVII.

Embodiment 454. The RNAi agent of embodiment 451 or 452, wherein at least two internucleoside linking groups of the RNAi antisense modified oligonucleotide are independently selected internucleoside linking groups of any of embodiments 311-385.

Embodiment 455. The RNAi agent of any of embodiments 450-454, wherein at least one of the five 3′-most internucleoside linking groups of the RNAi antisense modified oligonucleotide is an internucleoside linking group of Formula XVII.

Embodiment 456. The RNAi agent of any of embodiments 450-454, wherein at least two of the five 3′-most internucleoside linking groups of RNAi antisense modified oligonucleotide is an internucleoside linking group of Formula XVII.

Embodiment 457. The RNAi agent of any of embodiments 450-454, wherein at least one internucleoside linking group within the seed region of the RNAi antisense modified oligonucleotide is an internucleoside linking group of Formula XVII.

Embodiment 458. The RNAi agent of any of embodiments 450-457, wherein at least one region of the RNAi antisense modified oligonucleotide has structure A, B, C, D, or E.

Embodiment 459. The RNAi agent of embodiment 458, wherein at least one region having structure A, B, C, D, or E is within the seed region of the RNAi antisense modified oligonucleotide.

Embodiment 460. The RNAi agent of embodiment 458, wherein at least one region having structure A, B, C, D, or E is at the 3′ end of the RNAi antisense modified oligonucleotide.

Embodiment 461. The RNAi agent of any of embodiments 450-457, wherein at least one region of the RNAi antisense modified oligonucleotide has the formula (N_{g1})_{L1}(N_{g2})_{L2}(N_{g3})_{L3}, wherein each N_{g} is a nucleoside and each L is an internucleoside linking group; wherein each of L₁, and L₂ is a phosphodiester internucleoside linking group, a phosphorothioate internucleoside linking group, or an internucleoside linking group of Formula XVII:

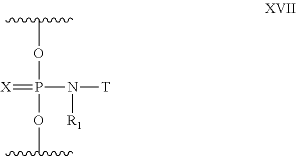

wherein L₃ is absent or is a phosphodiester internucleoside linking group, a phosphorothioate internucleoside linking group, or an internucleoside linking group of Formula XVII;
wherein at least one of L₁, L₂, and L₃ an internucleoside linking group of Formula XVII; and at least one of L₁, L₂, and L₃ is a phosphorothioate or a phosphodiester internucleoside linking group,
wherein independently for each internucleoside linking group of the modified oligonucleotide having Formula XVII:
   X is selected from O or S;
   R₁ is selected from H, C₁-C₆ alkyl, and substituted C₁-C₆ alkyl; and
   T is selected from SO₂R₂, C(═O)R₃, and P(═O)R₄R₅, wherein:
   R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl, substituted C₁-C₆ alkynyl, and a conjugate group;
   R₃ is selected from an aryl, a substituted aryl, CH₃, N(CH₃)₂, OCH₃ and a conjugate;
   R₄ is selected from OCH₃, OH, C₁-C₆ alkyl, substituted C₁-C₆ alkyl and a conjugate; and
R₅ is selected from OCH₃, OH, C₁-C₆ alkyl, and substituted C₁-C₆ alkyl.

Embodiment 462. The RNAi agent of embodiment 461, wherein at least one region having the formula (N_{g1})_{L1}(N_{g2})_{L2}(N_{g3})_{L3} is at the 3′ end of the RNAi antisense modified oligonucleotide.

Embodiment 463. The RNAi agent of embodiment 461, wherein at least one region having the formula (N_{g1})_{L1}(N_{g2})_{L2}(N_{g3})_{L3} is within the seed region of the RNAi antisense modified oligonucleotide.

Embodiment 464. The RNAi agent of embodiment 450 or 452-463 wherein at least one internucleoside linking group of the RNAi sense modified oligonucleotide is an internucleoside linking group of Formula XVII.

Embodiment 465. The RNAi agent of embodiment 464, wherein at least one of the first 5 internucleoside linking groups from the 5′-end of the RNAi sense modified oligonucleotide is an internucleoside linking group of Formula XVII.

Embodiment 466. The RNAi agent of any of embodiments 464-465, wherein at least one of the five 3′-most internucleoside linking groups of the RNAi sense modified oligonucleotide is an internucleoside linking group of Formula XVII.

Embodiment 467. The RNAi agent of any of embodiments 464-466, wherein at least one of the first 5 internucleoside linking groups from the 5′-end of the RNAi sense modified oligonucleotide and at least one of the five 3′-most linking groups of the RNAi sense modified oligonucleotide is an internucleoside linking group of Formula XVII.

Embodiment 468. The RNAi agent of any of embodiments 464-467, wherein at least one region of the RNAi sense modified oligonucleotide has structure A, B, C, D, or E.

Embodiment 469. The RNAi agent of embodiment 468, wherein at least one region having structure A, B, C, D, or E is at the 3′ end of the RNAi sense modified oligonucleotide.

Embodiment 470. The RNAi agent of embodiment 468, wherein at least one region having structure A, B, C, D, or E is at the 5′ end of the RNAi sense modified oligonucleotide.

Embodiment 471. The RNAi agent of any of embodiments 464-467, wherein at least one region of the RNAi sense modified oligonucleotide has the formula (N_{g1})_{L1}(N_{g2})_{L2}(N_{g3})_{L3}, wherein each N_{g} is a nucleoside and each L is an internucleoside linking group; wherein each of L₁, and L₂ is a phosphodiester internucleoside linking group, a phosphorothioate internucleoside linking group, or an internucleoside linking group of Formula XVII:

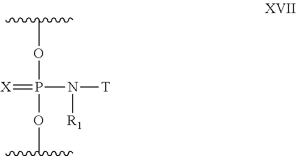

wherein L₃ is absent or is phosphodiester internucleoside linking group, a phosphorothioate internucleoside linking group, or an internucleoside linking group of Formula XVII;
wherein at least one of L₁, L₂, and L₃ an internucleoside linking group of Formula XVII; and at least one of L₁, L₂, and L₃ is a phosphorothioate or a phosphodiester internucleoside linking group,
   wherein independently for each internucleoside linking group of the modified oligonucleotide having Formula XVII:
   X is selected from O or S;
   R₁ is selected from H, C₁-C₆ alkyl, and substituted C₁-C₆ alkyl; and
   T is selected from SO₂R₂, C(═O)R₃, and P(═O)R₄R₅, wherein:
   R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl, substituted C₁-C₆ alkynyl, and a conjugate group;
   R₃ is selected from an aryl, a substituted aryl, CH₃, N(CH₃)₂, OCH₃ and a conjugate;
   R₄ is selected from OCH₃, OH, C₁-C₆ alkyl, substituted C₁-C₆ alkyl and a conjugate; and
      R₅ is selected from OCH₃, OH, C₁-C₆ alkyl, and substituted C₁-C₆ alkyl.

Embodiment 472. The RNAi agent of embodiment 471, wherein at least one region having the formula (N_{g1})_{L1}(N_{g2})_{L2}(N_{g3})_{L3} is at the 3′ end of the RNAi sense modified oligonucleotide.

Embodiment 473. The RNAi agent of embodiment 471, wherein at least one region having the formula (N_{g1})_{L1}(N_{g2})_{L2}(N_{g3})_{L3} is at the 5′ end of the RNAi sense modified oligonucleotide.

Embodiment 474. The modified oligonucleotide of any of embodiments 311-443, wherein each nucleoside of the modified oligonucleotide is a modified nucleoside comprising a modified sugar moiety.

Embodiment 475. The modified oligonucleotide of embodiment 474, wherein each modified sugar moiety is independently selected from a bicyclic sugar moiety and a 2′-substituted furanosyl sugar moiety.

Embodiment 476. The modified oligonucleotide of embodiment 474 or 475, wherein each modified sugar moiety comprises the same modification.

Embodiment 477. The modified oligonucleotide of any of embodiments 474-476, wherein each modified sugar moiety is selected from a 2′-OMe sugar moiety, a 2′-MOE sugar moiety, and a 2′-NMA sugar moiety.

Embodiment 478. The modified oligonucleotide of embodiment 476 or 477, wherein the three 3′-most nucleosides comprise a bicyclic sugar moiety, and the remaining nucleosides comprise a 2′-substituted furanosyl sugar moiety.

Embodiment 479. The modified oligonucleotide of embodiment 476 or 477, wherein the four 3′-most nucleosides comprise a bicyclic sugar moiety, and the remaining nucleosides comprise a 2′-substituted furanosyl sugar moiety.

Embodiment 480. The modified oligonucleotide of embodiment 476 or 477, wherein the five 3′-most nucleosides comprise a bicyclic sugar moiety, and the remaining nucleosides comprise a 2′-substituted furanosyl sugar moiety.

Embodiment 481. The modified oligonucleotide of embodiment 476 or 477, wherein the six 3′-most nucleosides comprise a bicyclic sugar moiety, and the remaining nucleosides comprise a 2′-substituted furanosyl sugar moiety.

Embodiment 482. The modified oligonucleotide of any of embodiments 476 or 478-481, wherein each bicyclic sugar moiety is selected from among cEt, LNA, and ENA.

Embodiment 483. The modified oligonucleotide of embodiment 482, wherein the bicyclic sugar moiety is cEt.

Embodiment 484. The modified oligonucleotide of any of embodiments 476 or 478-481, wherein the 2′-substituted furanosyl sugar moiety is selected from 2′-OMe, 2′-MOE, and 2′-F.

Embodiment 485. The modified oligonucleotide of any of embodiments 474-484, wherein at least one of the ten 5′-most linking groups of the modified oligonucleotide is an internucleoside linking group of Formula XVII.

Embodiment 486. The modified oligonucleotide of embodiment 485, wherein at least 2 of the ten 5′-most linking groups of the modified oligonucleotide are internucleoside linking groups of Formula XVII.

Embodiment 487. The modified oligonucleotide of embodiment 485, wherein at least 3 of the ten 5′-most linking groups of the modified oligonucleotide are internucleoside linking groups of Formula XVII.

Embodiment 488. The modified oligonucleotide of embodiment 485, wherein at least 4 of the ten 5′-most linking groups of the modified oligonucleotide are internucleoside linking groups of Formula XVII.

Embodiment 489. The modified oligonucleotide of embodiment 485, wherein at least 5 of the ten 5′-most linking groups of the modified oligonucleotide are internucleoside linking groups of Formula XVII.

Embodiment 490. The modified oligonucleotide of embodiment 485, wherein at least 6 of the ten 5′-most linking groups of the modified oligonucleotide are internucleoside linking groups of Formula XVII.

Embodiment 491. The modified oligonucleotide of embodiment 485, wherein the two 5′-most internucleoside linking groups are internucleoside linking groups of Formula XVII.

Embodiment 492. The modified oligonucleotide of any of embodiments 478-491, wherein at least one of the ten 3′-most internucleoside linking groups of the modified oligonucleotide is an internucleoside linking group of Formula XVII.

Embodiment 493. The modified oligonucleotide of embodiment 492, wherein at least 2 of the ten 3′-most internucleoside linking groups of the modified oligonucleotide are internucleoside linking groups of Formula XVII.

Embodiment 494. The modified oligonucleotide of embodiment 492, wherein at least 3 of the ten 3′-most internucleoside linking groups are internucleoside linking groups of Formula XVII.

Embodiment 495. The modified oligonucleotide of embodiment 492, wherein at least 4 of the ten 3′-most internucleoside linking groups are internucleoside linking groups of Formula XVII.

Embodiment 496. The modified oligonucleotide of embodiment 492, wherein at least 5 of the ten 3′-most internucleoside linking groups are internucleoside linking groups of Formula XVII.

Embodiment 497. The modified oligonucleotide of embodiment 492, wherein at least 6 of the ten 3′-most internucleoside linking groups are internucleoside linking groups of Formula XVII.

Embodiment 498. The modified oligonucleotide of embodiment 492, wherein the two 3′-most internucleoside linking groups of the modified oligonucleotide are internucleoside linking groups of Formula XVII.

Embodiment 499. The modified oligonucleotide of any of embodiments 474-484, wherein the modified oligonucleotide comprises at least one block of at least 3 consecutive internucleoside linking groups of Formula XVII.

Embodiment 500. The modified oligonucleotide of any of embodiments 474-484, wherein the modified oligonucleotide comprises at least one block of at least 4 consecutive internucleoside linking groups of Formula XVII.

Embodiment 501. The modified oligonucleotide of any of embodiments 474-484, wherein the modified oligonucleotide comprises at least one block of at least 5 consecutive internucleoside linking groups of Formula XVII.

Embodiment 502. The modified oligonucleotide of any of embodiments 474-484, wherein the modified oligonucleotide comprises at least one block of at least 6 consecutive internucleoside linking groups of Formula XVII.

Embodiment 503. The modified oligonucleotide of any of embodiments 499-502, wherein at least one block of consecutive internucleoside linking groups of Formula XVII is at the 5′ end of the modified oligonucleotide.

Embodiment 504. The modified oligonucleotide of any of embodiments 499-502, wherein at least one block of consecutive internucleoside linking groups of Formula XVII is at the 3′ end of the modified oligonucleotide.

Embodiment 505. The modified oligonucleotide of any of embodiments 311-443, wherein the modified oligonucleotide comprises a deoxy region consisting of 6-11 linked nucleosides wherein each nucleoside of the deoxy region is either a modified nucleoside or a stereo-standard DNA nucleoside and wherein at least 3 contiguous nucleosides of the deoxy region are stereo-standard DNA nucleosides and not more than three nucleosides of the deoxy region are modified nucleosides.

Embodiment 506. The modified oligonucleotide of embodiment 505, wherein at least 4 contiguous nucleosides of the deoxy region are stereo-standard DNA nucleosides.

Embodiment 507. The modified oligonucleotide of embodiment 505, wherein at least 5 contiguous nucleosides of the deoxy region are stereo-standard DNA nucleosides.

Embodiment 508. The modified oligonucleotide of embodiment 505, wherein at least 6 contiguous nucleosides of the deoxy region are stereo-standard DNA nucleosides.

Embodiment 509. The modified oligonucleotide of embodiment 505, wherein at least 7 contiguous nucleosides of the deoxy region are stereo-standard DNA nucleosides.

Embodiment 510. The modified oligonucleotide of embodiment 505, wherein at least 8 contiguous nucleosides of the deoxy region are stereo-standard DNA nucleosides.

Embodiment 511. The modified oligonucleotide of any of embodiments 505-510, wherein the deoxy region consists of 8-10 linked nucleosides.

Embodiment 512. The modified oligonucleotide of any of embodiments 505-510, wherein the deoxy region consists of 9 linked nucleosides.

Embodiment 513. The modified oligonucleotide of any of embodiments 505-510, wherein the deoxy region consists of 10 linked nucleosides.

Embodiment 514. The modified oligonucleotide of any of embodiments 505-510, wherein the deoxy region consists of 11 linked nucleosides.

Embodiment 515. The modified oligonucleotide of any of embodiments 505-510, wherein at least 6 nucleosides of the deoxy region are stereo-standard DNA nucleosides.

Embodiment 516. The modified oligonucleotide of any of embodiments 505-510, wherein at least 7 nucleosides of the deoxy region are stereo-standard DNA nucleosides.

Embodiment 517. The modified oligonucleotide of any of embodiments 505-510, wherein at least 8 nucleosides of the deoxy region are stereo-standard DNA nucleosides.

Embodiment 518. The modified oligonucleotide of any of embodiments 505-510, wherein at least 9 nucleosides of the deoxy region are stereo-standard DNA nucleosides.

Embodiment 519. The modified oligonucleotide of any of embodiments 505-518 wherein two nucleosides of the deoxy region are modified nucleosides.

Embodiment 520. The modified oligonucleotide of any of embodiments 505-518 wherein one nucleoside of the deoxy region is a modified nucleoside.

Embodiment 521. The modified oligonucleotide of any of embodiments 505-520 wherein at least one modified nucleoside of the deoxy region is a stereo-standard modified nucleoside or bicyclic nucleoside selected from a β-D-LNA nucleoside, an α-L-LNA nucleoside, an ENA nucleoside, a cEt nucleoside, a 2′-MOE nucleoside, a 2′-OMe nucleoside, a 2′-F nucleoside, and a 5′-alkyl nucleoside.

Embodiment 522. The modified oligonucleotide of any of embodiments 505-520 wherein at least one modified nucleoside of the deoxy region is stereo-non-standard nucleoside.

Embodiment 523. The modified oligonucleotide of embodiment 522 wherein the at least one is stereo-non-standard nucleoside of the deoxy region is a stereo-non-standard DNA nucleoside.

Embodiment 524. The modified oligonucleotide of embodiment 523 wherein the stereo-non-standard DNA nucleoside is selected from a stereo-non-standard DNA nucleoside having: Formula I, Formula II, Formula III, Formula IV, Formula V, Formula VI, and Formula VII.

Embodiment 525. The modified oligonucleotide of embodiment 524 wherein the stereo-non-standard DNA nucleoside is selected from a stereo-non-standard DNA nucleoside having: Formula V and Formula II.

Embodiment 526. The modified oligonucleotide of embodiment 525 wherein at least one stereo-non-standard nucleoside of the deoxy region is a substituted stereo-non-standard nucleoside.

Embodiment 527. The modified oligonucleotide of embodiment 526 wherein at least one substituted stereo-non-standard nucleoside has a 2′-substituent selected from: 2′-MOE, 2′-OMe, 2′-F, or 2′-OH.

Embodiment 528. The modified oligonucleotide of any of embodiments 505-527, wherein the 2^{nd} nucleoside from the 5′-end of the deoxy region is a modified nucleoside.

Embodiment 529. The modified oligonucleotide of any of embodiments 505-527, wherein the 3^{rd} nucleoside from the 5′-end of the deoxy region is a modified nucleoside.

Embodiment 530. The modified oligonucleotide of any of embodiments 505-527, wherein the 4^{th} nucleoside from the 5′-end of the deoxy region is a modified nucleoside.

Embodiment 531. The modified oligonucleotide of any of embodiments 528-530, wherein the modified nucleoside in the deoxy region is a 2′-OMe nucleoside.

Embodiment 532. The modified oligonucleotide of any of embodiments 505-518, wherein each nucleoside of the deoxy region is a stereo-standard DNA nucleoside.

Embodiment 533. The modified oligonucleotide of any of embodiments 505-532 wherein at least one internucleoside linking group within the deoxy region is an internucleoside linking group of Formula XVII.

Embodiment 534. The modified oligonucleotide of any of embodiments 505-532, wherein the internucleoside linking group linking the 1^{It} and 2^{nd} nucleosides of the deoxy region as counted from the 5′-end of the deoxy region is an internucleoside linking group of Formula XVII.

Embodiment 535. The modified oligonucleotide of any of embodiments 505-534, wherein the internucleoside linking group linking the 2^{nd} and 3^{rd} nucleosides of the deoxy region as counted from the 5′-end of the deoxy region is an internucleoside linking group of Formula XVII.

Embodiment 536. The modified oligonucleotide of any of embodiments 505-535, wherein the internucleoside linking group linking the 3^{rd} and 4^{h} nucleosides of the deoxy region as counted from the 5′-end of the deoxy region is an internucleoside linking group of Formula XVII.

Embodiment 537. The modified oligonucleotide of any of embodiments 505-536, wherein the internucleoside linking group linking the 4^{th} and 5^{th} nucleosides of the deoxy region as counted from the 5′-end of the deoxy region is an internucleoside linking group of Formula XVII.

Embodiment 538. The modified oligonucleotide of any of embodiments 505-537, wherein one internucleoside linking group in the deoxy region is a linking group of Formula XVII and the other internucleoside linking groups of the deoxy region are each phosphodiester or phosphorothioate internucleoside linking groups.

Embodiment 539. The modified oligonucleotide of any of embodiments 505-537, wherein two internucleoside linking groups in the deoxy region are linking groups of Formula XVII and the other internucleoside linking groups of the deoxy region are each phosphodiester or phosphorothioate internucleoside linking groups.

Embodiment 540. The modified oligonucleotide of any of embodiments 505-537, wherein three internucleoside linking groups in the deoxy region are linking groups linking groups of Formula XVII and the other internucleoside linking groups of the deoxy region are each phosphodiester or phosphorothioate internucleoside linking groups.

Embodiment 541. The modified oligonucleotide of any of embodiments 505-540, wherein the deoxy region comprises at least one region having structure A, B, C, D, or E.

Embodiment 542. The modified oligonucleotide of embodiment 541, wherein the region having structure A, B, C, D, or E is at the 3′ end of the deoxy region.

Embodiment 543. The modified oligonucleotide of embodiment 541, wherein the region having structure A, B, C, D, or E is at the 5′ end of the deoxy region.

Embodiment 544. The modified oligonucleotide of any of embodiments 505-540, wherein the deoxy region comprises at least one region having the formula (N_{g1})_{L1} (N_{g2})_{L2}(N_{g3})_{L3}, wherein each N_{g} is a nucleoside and each L is an internucleoside linking group; wherein each of L₁, and L₂ is a phosphodiester internucleoside linking group, a phosphorothioate internucleoside linking group, or an internucleoside linking group of Formula XVII:

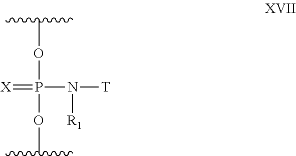

wherein L₃ is absent or is a phosphodiester internucleoside linking group, a phosphorothioate internucleoside linking group, or an internucleoside linking group of Formula XVII;
wherein at least one of L₁, L₂, and L₃ an internucleoside linking group of Formula XVII; and at least one of L₁, L₂, and L₃ is a phosphorothioate or a phosphodiester internucleoside linking group,
   wherein independently for each internucleoside linking group of the modified oligonucleotide having Formula XVII:
   X is selected from O or S;
   R₁ is selected from H, C₁-C₆ alkyl, and substituted C₁-C₆ alkyl; and
   T is selected from SO₂R₂, C(═O)R₃, and P(═O)R₄R₅, wherein:
   R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl, substituted C₁-C₆ alkynyl, and a conjugate group;
   R₃ is selected from an aryl, a substituted aryl, CH₃, N(CH₃)₂, OCH₃ and a conjugate;
   R₄ is selected from OCH₃, OH, C₁-C₆ alkyl, substituted C₁-C₆ alkyl and a conjugate; and
      R₅ is selected from OCH₃, OH, C₁-C₆ alkyl, and substituted C₁-C₆ alkyl.

Embodiment 545. The modified oligonucleotide of any of embodiments 544, wherein the region having the formula (N_{g1})_{L1}(N_{g2})_{L2}(N_{g3})_{L3} is at the 3′ end of the deoxy region.

Embodiment 546. The modified oligonucleotide of any of embodiments 544, wherein the region having the formula (N_{g1})_{L1}(N_{g2})_{L2}(N_{g3})_{L3} is at the 5′ end of the deoxy region.

Embodiment 547. The modified oligonucleotide of any of embodiments 505-546 wherein the deoxy region is flanked on the 5′ side by a 5′-region consisting of 1-6 linked 5′-region nucleosides and on the 3′ side by a 3′-region consisting of 1-6 linked 3′-region nucleosides; wherein
the 3′-most nucleoside of the 5′-region is a modified nucleoside; and
the 5′-most nucleoside of the 3′-region is a modified nucleoside.

Embodiment 548. The modified oligonucleotide of embodiment 547, wherein the deoxy region consists of 7-11 linked nucleosides, and has the formula: (N_{d1})_{L1}(N_{d2})_{L2}(N_{d3})_{L3}(N_{d4})_{L4}[(N_{d})_{L5}]_{q};
wherein N_{d1}, N_{d2}, N_{d3}, N_{d4} are independently selected from among a stereo-standard DNA nucleoside, a stereo-non-standard DNA nucleoside, or a 2′-substituted nucleoside; with the proviso that no more than one of N_{d1}, N_{d2}, N_{d3}, or N_{d4} is a 2′-substituted nucleoside;
each N_{d} is independently selected from among a stereo-standard DNA nucleoside and a stereo-non-standard DNA nucleoside;
q is from 3-8;
wherein each of L₁, L₂, L₃, L₄, and each L₅ is an internucleoside linkage;
wherein at least two of L₁, L₂, L₃, L₄ are internucleoside linkages of Formula XVII.

Embodiment 549. The modified oligonucleotide of embodiment 548, wherein one of N_{d1}, N_{d2}, N_{d3}, or N_{d4} is a 2′-substituted nucleoside.

Embodiment 550. The modified oligonucleotide of embodiment 549, wherein the 2′-substituted nucleoside is a 2′-OMe nucleoside.

Embodiment 551. The modified oligonucleotide of embodiment 550, wherein the 2′-OMe nucleoside is a stereo-standard 2′-OMe nucleoside.

Embodiment 552. The modified oligonucleotide of any of embodiments 548-551, wherein the 2′-substituted nucleoside is N_{d2}.

Embodiment 553. The modified oligonucleotide of embodiment 548, wherein each of N_{d1}, N_{d2}, N_{d3}, N_{d4} and each N_{d} is a DNA nucleoside.

Embodiment 554. The modified oligonucleotide of embodiment 553, wherein each DNA nucleoside is a stereo-standard DNA nucleoside.

Embodiment 555. The modified oligonucleotide of any of embodiments 548-554, wherein L₁ and L₂ are internucleoside linkages of Formula XVII.

Embodiment 556. The modified oligonucleotide of any of embodiments 548-554, wherein L₂ and L₃ are internucleoside linkages of Formula XVII.

Embodiment 557. The modified oligonucleotide of any of embodiments 548-554, wherein L₃ and L₄ are internucleoside linkages of Formula XVII.

Embodiment 558. The modified oligonucleotide of any of embodiments 548-554, wherein L₁, L₂, and L₃ are internucleoside linkages of Formula XVII.

Embodiment 559. The modified oligonucleotide of any of embodiments 548-554, wherein L₂, L₃, and L₄,are internucleoside linkages of Formula XVII.

Embodiment 560. The modified oligonucleotide of any of embodiments 548-554, wherein L₁, L₂, L₃, and L₄ are internucleoside linkages of Formula XVII.

Embodiment 561. The modified oligonucleotide of any of embodiments 547-560, wherein the 5′-region consists of 2-5 linked nucleosides.

Embodiment 562. The modified oligonucleotide of embodiment 561, wherein the 5′-region consists of 3 linked nucleosides.

Embodiment 563. The modified oligonucleotide of embodiment 561, wherein the 5′-region consists of 5 linked nucleosides.

Embodiment 564. The modified oligonucleotide of any of embodiments 547-563 wherein each nucleoside of the 5′-region is a modified nucleoside.

Embodiment 565. The modified oligonucleotide of any of embodiments 547-564, wherein each nucleoside of the 5′-region is a modified nucleoside comprising a modified sugar.

Embodiment 566. The modified oligonucleotide of any of embodiments 547-565, wherein at least one nucleoside of the 5′-region comprises a 2′-substituted furanosyl sugar moiety.

Embodiment 567. The modified oligonucleotide of any of embodiments 547-566, wherein each nucleoside of the 5′-region comprises a 2′-substituted furanosyl sugar moiety.

Embodiment 568. The modified oligonucleotide of any or embodiments 547-567, wherein each 2′-substituted furanosyl sugar moiety of the 5′-region has a 2′-substituent selected from among 2′-MOE, 2′-OMe, and 2′-NMA.

Embodiment 569. The modified oligonucleotide of any of embodiments 547-566 or 568, wherein at least one nucleoside of the 5′-region comprises a bicyclic furanosyl sugar moiety.

Embodiment 570. The modified oligonucleotide of any of embodiments 547-566 or 568-569, wherein each nucleoside of the 5′-region comprises a bicyclic furanosyl sugar moiety.

Embodiment 571. The modified oligonucleotide of embodiment 569 or 570, wherein each bicyclic sugar moiety of the 5′-region is selected from among cEt, LNA, and ENA.

Embodiment 572. The modified oligonucleotide of embodiment 571, wherein each bicyclic sugar moiety of the 5′-region is a cEt sugar moiety.

Embodiment 573. The modified oligonucleotide of any of embodiments 547-563, 566 or 569, wherein at least one nucleoside of the 5′ region is a stereo-standard DNA nucleoside.

Embodiment 574. The modified oligonucleotide of any of embodiments 547-572, wherein at least one nucleoside of the 5′ region is a stereo-non-standard nucleoside.

Embodiment 575. The modified oligonucleotide of any of embodiments 547-574, wherein each nucleobase of the 5′-region is independently selected from among thymine, uracil, guanine, cytosine, 5-methylcytosine, and adenine.

Embodiment 576. The modified oligonucleotide of any of embodiments 547-575, wherein the 3′-region consists of 2-5 linked nucleosides.

Embodiment 577. The modified oligonucleotide of embodiment 576, wherein the 3′-region consists of 3 linked nucleosides.

Embodiment 578. The modified oligonucleotide of embodiment 576, wherein the 3′-region consists of 5 linked nucleosides.

Embodiment 579. The modified oligonucleotide of any of embodiments 547-578, wherein each nucleoside of the 3′-region is a modified nucleoside.

Embodiment 580. The modified oligonucleotide of any of embodiments 547-578, wherein each nucleoside of the 3′-region is a modified nucleoside comprising a modified sugar.

Embodiment 581. The modified oligonucleotide of any of embodiments 547-580, wherein at least one nucleoside of the 3′-region comprises a 2′-substituted furanosyl sugar moiety.

Embodiment 582. The modified oligonucleotide of any of embodiments 547-581, wherein each nucleoside of the 3′-region comprises a 2′-substituted furanosyl sugar moiety.

Embodiment 583. The modified oligonucleotide of any or embodiments 547-582, wherein each 2′-substituted furanosyl sugar moiety of the 3′-region has a 2′-substituent selected from among 2′-MOE, 2′-OMe, and 2′-NMA.

Embodiment 584. The modified oligonucleotide of any of embodiments 547-581 or 583, wherein at least one nucleoside of the 3′-region comprises a bicyclic furanosyl sugar moiety.

Embodiment 585. The modified oligonucleotide of any of embodiments 547-580 or 584, wherein each nucleoside of the 3′-region comprises a bicyclic furanosyl sugar moiety.

Embodiment 586. The modified oligonucleotide of embodiment 584 or 585, wherein each bicyclic sugar moiety of the 3′-region is selected from among cEt, LNA, and ENA.

Embodiment 587. The modified oligonucleotide of embodiment 586, wherein each bicyclic sugar moiety of the 3′-region is a cEt sugar moiety.

Embodiment 588. The modified oligonucleotide of any of embodiments 547-578, 581 or 584, wherein at least one nucleoside of the 3′ region is a stereo-standard DNA nucleoside.

Embodiment 589. The modified oligonucleotide of any of embodiments 547-588, wherein at least one nucleoside of the 3′ region is a stereo-non-standard nucleoside.

Embodiment 590. The modified oligonucleotide of any of embodiments 547-589, wherein each nucleobase of the 3′-region is independently selected from among thymine, uracil, guanine, cytosine, 5-methylcytosine, and adenine.

Embodiment 591. The modified oligonucleotide of any of embodiments 547-590 wherein the modified oligonucleotide is a gapmer.

Embodiment 592. The modified oligonucleotide of any of embodiments 311-432, wherein the modified oligonucleotide is a CRISPR compound.

Embodiment 593. The modified oligonucleotide of embodiment 592, wherein the CRISPR compound consists of 20-50 linked nucleosides.

Embodiment 594. The modified oligonucleotide of embodiment 592, wherein the CRISPR compound consists of 29-32 linked nucleosides.

Embodiment 595. The modified oligonucleotide of any of embodiments 311-432, wherein the modified oligonucleotide is an artificial mRNA compound.

Embodiment 596. The artificial mRNA compound of embodiment 595, wherein the artificial mRNA oligonucleotide consists of 17-3000 linked nucleosides.

Embodiment 597. The artificial mRNA compound of embodiment 595 or 596, wherein the artificial mRNA oligonucleotide encodes a protein.

Embodiment 598. The modified oligonucleotide of any of embodiments 396-597, wherein each X is O.

Embodiment 599. The modified oligonucleotide of any of embodiments 396-597, wherein each X is S.

Embodiment 600. The modified oligonucleotide of any of embodiments 396-599, wherein at least one R₁ is H.

Embodiment 601. The modified oligonucleotide of any of embodiments 396-599, wherein at least one R₁ is a C₁-C₆ alkyl.

Embodiment 602. The modified oligonucleotide of embodiment 601, wherein the at least one R₁ is methyl.

Embodiment 603. The modified oligonucleotide of any of embodiments 396-602, at least one R₁ is a substituted C₁-C₆ alkyl.

Embodiment 604. The modified oligonucleotide of any of embodiments 396-603, wherein at least one T comprises a conjugate group.

Embodiment 605. The modified oligonucleotide of embodiment 604, wherein the conjugate group comprises a carbohydrate or carbohydrate cluster.

Embodiment 606. The modified oligonucleotide of embodiment 604 or 605, wherein the conjugate group comprises at least one GalNAc.

Embodiment 607. The modified oligonucleotide of embodiment 604, wherein the conjugate group comprises a C₁₀-C₂₀ alkyl chain.

Embodiment 608. The modified oligonucleotide of embodiment 607, wherein the conjugate group comprises C₁₆ alkyl.

Embodiment 609. The modified oligonucleotide of any of embodiments 396-603, wherein at least one T does not comprise a conjugate group.

Embodiment 610. The modified oligonucleotide of any of embodiments 396-603, wherein each T does not comprise a conjugate group.

Embodiment 611. The modified oligonucleotide of any of embodiments 396-603, wherein at least one T is SO₂R₂.

Embodiment 612. The modified oligonucleotide of embodiment 611, wherein R₂ is an aryl.

Embodiment 613. The modified oligonucleotide of embodiment 611, wherein R₂ is a substituted aryl.

Embodiment 614. The modified oligonucleotide of embodiment 611, wherein R₂ is a heterocycle.

Embodiment 615. The modified oligonucleotide of embodiment 611, wherein R₂ is a substituted heterocycle.

Embodiment 616. The modified oligonucleotide of embodiment 611, wherein R₂ is an aromatic heterocycle.

Embodiment 617. The modified oligonucleotide of embodiment 611, wherein R₂ is a substituted aromatic heterocycle.

Embodiment 618. The modified oligonucleotide of embodiment 611, wherein R₂ is a diazole.

Embodiment 619. The modified oligonucleotide of embodiment 611, wherein R₂ is a substituted diazole.

Embodiment 620. The modified oligonucleotide of embodiment 611, wherein R₂ is an amine.

Embodiment 621. The modified oligonucleotide of embodiment 611, wherein R₂ is a substituted amine.

Embodiment 622. The modified oligonucleotide of embodiment 611, wherein R₂ is a C₁-C₆ alkoxy, C₁-C₆ alkenyl, or C₁-C₆ alkynyl.

Embodiment 623. The modified oligonucleotide of embodiment 611, wherein R₂ is C₁-C₂₀, C₁-C₆, C₂-C₂₀, C₂-C₆, or C₁₀-C₂₀ alkyl.

Embodiment 624. The modified oligonucleotide of embodiment 611, wherein R₂ is substituted C₁-C₂₀, C₁-C₆, C₂-C₂0, C₂-C₆, or C₁₀-C₂₀ alkyl.

Embodiment 625. The modified oligonucleotide of embodiment 611, wherein R₂ comprises a carbohydrate or carbohydrate cluster.

Embodiment 626. The modified oligonucleotide of embodiment 611, wherein R₂ comprises at least one GalNAc.

Embodiment 627. The modified oligonucleotide of embodiment 611, wherein T is:

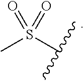 [SMILES: *S(C)(=O)=O]

Embodiment 628. The modified oligonucleotide of embodiment 611, wherein T is:

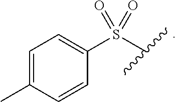 [SMILES: *S(=O)(=O)c1ccc(C)cc1]

Embodiment 629. The modified oligonucleotide of embodiment 611, wherein T is:

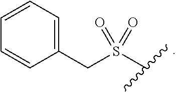 [SMILES: *S(=O)(=O)Cc1ccccc1]

Embodiment 630. The modified oligonucleotide of embodiment 611, wherein T is:

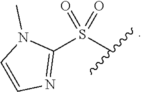 [SMILES: *S(=O)(=O)c1nccn1C]

Embodiment 631. The modified oligonucleotide of embodiment 611, wherein T is:

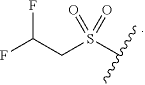 [SMILES: *S(=O)(=O)CC(F)F]

Embodiment 632. The modified oligonucleotide of embodiment 611, wherein T is:

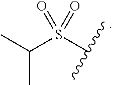 [SMILES: *S(=O)(=O)C(C)C]

Embodiment 633. The modified oligonucleotide of embodiment 611, wherein T is:

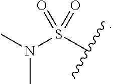 [SMILES: *S(=O)(=O)N(C)C]

Embodiment 634. The modified oligonucleotide of embodiment 611, wherein T is:

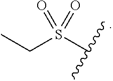 [SMILES: *S(=O)(=O)CC]

Embodiment 635. The modified oligonucleotide of embodiment 611, wherein T is:

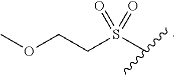 [SMILES: *S(=O)(=O)CCOC]

Embodiment 636. The modified oligonucleotide of embodiment 611, wherein T is:

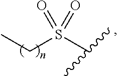

wherein n is from 2 to 20.

Embodiment 637. The modified oligonucleotide of embodiment 636, wherein n is 15.

Embodiment 638. The modified oligonucleotide of any of embodiments 396-603, wherein at least one T is C(═O)R₃.

Embodiment 639. The modified oligonucleotide of embodiment 638, wherein R₃ is an aryl.

Embodiment 640. The modified oligonucleotide of embodiment 638, wherein R₃ is a substituted aryl.

Embodiment 641. The modified oligonucleotide of embodiment 638, wherein R₃ is CH₃.

Embodiment 642. The modified oligonucleotide of embodiment 638, wherein R₃ is N(CH₃)2.

Embodiment 643. The modified oligonucleotide of embodiment 638, wherein R₃ is OCH₃.

Embodiment 644. The modified oligonucleotide of embodiment 638, wherein R₃ is a C₁-C₆ alkoxy.

Embodiment 645. The modified oligonucleotide of embodiment 638, wherein R₃ is C₁-C₂₀, C₁-C₆, C₂-C₂0, C₂-C₆, or C₁₀-C₂₀ alkyl.

Embodiment 646. The modified oligonucleotide of embodiment 638, wherein R₃ is substituted C₁-C₂₀, C₁-C₆, C₂-C₂₀, C₂-C₆, or C₁₀-C₂₀ alkyl.

Embodiment 647. The modified oligonucleotide of embodiment 638, wherein R₃ comprises a carbohydrate or carbohydrate cluster.

Embodiment 648. The modified oligonucleotide of embodiment 638, wherein R₂₃ comprises at least one GalNAc.

Embodiment 649. The modified oligonucleotide of embodiment 638, wherein T is:

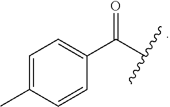 [SMILES: *C(=O)c1ccc(C)cc1]

Embodiment 650. The modified oligonucleotide of embodiment 638, wherein T is:

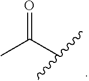 [SMILES: *C(C)=O]

Embodiment 651. The modified oligonucleotide of embodiment 638, wherein T is:

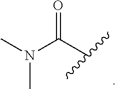 [SMILES: *C(=O)N(C)C]

Embodiment 652. The modified oligonucleotide of embodiment 638, wherein T is:

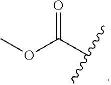 [SMILES: *C(=O)OC]

Embodiment 653. The modified oligonucleotide of embodiment 638, wherein T is:

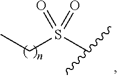 [SMILES: *S(=O)(=O)CC]

n wherein n is from 2 to 20.

Embodiment 654. The modified oligonucleotide of embodiment 653, wherein n is 15.

Embodiment 655. The modified oligonucleotide of any of embodiments 396-603, wherein at least one T is P(═O)R₄R₅.

Embodiment 656. The modified oligonucleotide of embodiment 655, wherein R₄ is OCH₃.

Embodiment 657. The modified oligonucleotide of embodiment 655, wherein R₄ is OH.

Embodiment 658. The modified oligonucleotide of embodiment 655, wherein R₄ is C₁-C₆ alkyl.

Embodiment 659. The modified oligonucleotide of embodiment 655, wherein R₄ is substituted C₁-C₆ alkyl.

Embodiment 660. The modified oligonucleotide of embodiment 655, wherein R₄ is C₁-C₂0, C₁-C₆, C₂-C₂₀, C₂-C₆, or C₁₀-C₂₀ alkyl.

Embodiment 661. The modified oligonucleotide of embodiment 655, wherein R₄ is substituted C₁-C₂₀, C₁-C₆, C₂-C₂₀, C₂-C₆, or C₁₀-C₂₀ alkyl.

Embodiment 662. The modified oligonucleotide of embodiment 655, wherein R₄ comprises a carbohydrate or carbohydrate cluster.

Embodiment 663. The modified oligonucleotide of embodiment 655, wherein R₄ comprises at least one GalNAc.

Embodiment 664. The modified oligonucleotide of any of embodiments 655-663, wherein R₅ is OCH₃.

Embodiment 665. The modified oligonucleotide of any of embodiments 655-663, wherein R₅ is OH.

Embodiment 666. The modified oligonucleotide of any of embodiments 655-663, wherein R₅ is C₁-C₆ alkyl.

Embodiment 667. The modified oligonucleotide of any of embodiments 655-663, wherein R₅ is substituted C₁-C₆ alkyl.

Embodiment 668. The modified oligonucleotide of embodiment 655, wherein T is:

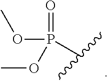 [SMILES: *P(=O)(OC)OC]

Embodiment 669. The modified oligonucleotide of embodiment 655, wherein T is:

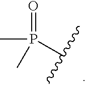 [SMILES: *P(C)(C)=O]

Embodiment 670. The modified oligonucleotide of embodiment 655, wherein T is:

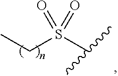

wherein n is from 2 to 20.

Embodiment 671. The modified oligonucleotide of embodiment 670, wherein n is 15.

Embodiment 672. A chirally enriched population of modified oligonucleotides of any of embodiments 311-671, wherein the population is enriched for modified oligonucleotides comprising at least one particular internucleoside linking group having a particular stereochemical configuration.

Embodiment 673. The chirally enriched population of modified oligonucleotides of embodiment 672, wherein the particular internucleoside linking group having a particular stereochemical configuration is an internucleoside linking group of Formula XVIII, as indicated in Formula XVIIIa and XVIIIb below:

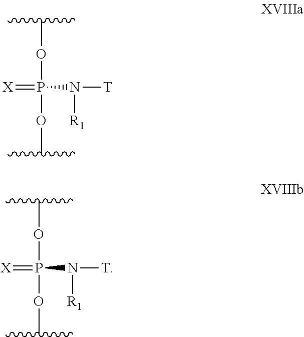

Embodiment 674. The chirally enriched population of modified oligonucleotides of embodiment 672, wherein the particular internucleoside linking group having a particular stereochemical configuration is a phosphorothioate internucleoside linking group.

Embodiment 675. The chirally enriched population of any of embodiments 672-674, wherein the population is enriched for modified oligonucleotides comprising at least one particular internucleoside linkage having the (Sp) configuration.

Embodiment 676. The chirally enriched population of any of embodiments 672-675, wherein the population is enriched for modified oligonucleotides comprising at least one particular internucleoside linkage having the (Rp) configuration.

Embodiment 677. The chirally enriched population of embodiment 672, wherein the population is enriched for modified oligonucleotides having a particular, independently selected stereochemical configuration at each chiral internucleoside linkage.

Embodiment 678. The chirally enriched population of any of embodiments 672-677, wherein the population is enriched for modified oligonucleotides having the (Sp) configuration at each chiral internucleoside linkage.

Embodiment 679. The chirally enriched population of any of embodiments 672-677, wherein the population is enriched for modified oligonucleotides having the (Rp) configuration at each chiral internucleoside linkage.

Embodiment 680. The chirally enriched population of any of embodiments 672-677, wherein the population is enriched for modified oligonucleotides having the (Rp) configuration at one particular chiral internucleoside linkage and the (Sp) configuration at each of the remaining chiral internucleoside linkages.

Embodiment 681. The chirally enriched population of embodiment 673, wherein each phosphorothioate internucleoside linkage is stereorandom.

Embodiment 682. The modified oligonucleotide of any of embodiments 311-681, wherein the nucleobase sequence of the modified oligonucleotide is complementary to a target nucleic acid.

Embodiment 683. The modified oligonucleotide of embodiment 682, wherein the nucleobase sequence of the modified oligonucleotide is at least 80% complementary to the target nucleic acid.

Embodiment 684. The modified oligonucleotide of embodiment 682, wherein the nucleobase sequence of the modified oligonucleotide is at least 85% complementary to the target nucleic acid.

Embodiment 685. The modified oligonucleotide of embodiment 682, wherein the nucleobase sequence of the modified oligonucleotide is at least 90% complementary to the target nucleic acid.

Embodiment 686. The modified oligonucleotide of embodiment 682, wherein the nucleobase sequence of the modified oligonucleotide is at least 95% complementary to the target nucleic acid.

Embodiment 687. The modified oligonucleotide of embodiment 682, wherein the nucleobase sequence of the modified oligonucleotide is 100% complementary to the target nucleic acid.

Embodiment 688. The modified oligonucleotide of any of embodiments 682-687, wherein the target nucleic acid is a target RNA.

Embodiment 689. The modified oligonucleotide of embodiment 688, wherein the target RNA is selected from: an mRNA, a pre-mRNA, a microRNA, and a non-coding RNA.

Embodiment 690. The modified oligonucleotide of embodiment 688, wherein the target RNA is not a microRNA.

Embodiment 691. The modified oligonucleotide of any of embodiments 311-690, wherein the modified oligonucleotide is not complementary to miR-21.

Embodiment 692. The modified oligonucleotide of any of embodiments 311-691, comprising a conjugate group.

Embodiment 693. The modified oligonucleotide of embodiment 692, wherein the conjugate group comprises at least one GalNAc.

Embodiment 694. The modified oligonucleotide of embodiment 692 or 693, wherein the conjugate group comprises 1-5 linker-nucleosides.

Embodiment 695. A pharmaceutical composition comprising the modified oligonucleotide of any of embodiments 311-694 and a pharmaceutically acceptable carrier or diluent.

Embodiment 696. A method comprising contacting a cell with the modified oligonucleotide or pharmaceutical composition of any of embodiments 311-695.

Embodiment 697. A method of modulating the amount or activity of a target nucleic acid in a cell, comprising contacting the cell with the modified oligonucleotide or pharmaceutical composition of any of embodiments 311-695 and thereby modulating the amount or activity of the target nucleic acid.

Embodiment 698. A method of modulating the amount or activity of a target nucleic acid in a cell, comprising contacting the cell with the modified oligonucleotide or pharmaceutical composition of any of embodiments 311-695.

Embodiment 699. The method of embodiments 696-698, wherein the amount or activity of a target nucleic acid is reduced.

Embodiment 700. The method of embodiments 696-698, wherein the amount or activity of a target nucleic acid is increased.

Embodiment 701. The method of embodiment 700, wherein the target protein is encoded by a target nucleic acid comprising at least one translation suppression element and wherein the modified oligonucleotide is complementary to a target site within a translation suppression element region of the target nucleic acid.

Embodiment 702. The method of embodiment 701, wherein the translation suppression element region comprises at least one stem-loop structure.

Embodiment 703. Use of the modified oligonucleotide or composition of any of embodiments 311-695 for treatment of a disease or condition.

Embodiment 704. Use of the modified oligonucleotide or composition of any of embodiments 311-695 for a preparation of a medicament for treatment of a disease or condition.

Embodiment 705. An antisense agent comprising a modified oligonucleotide consisting of 12-70 linked nucleosides linked through internucleoside linking groups, wherein at least one nucleoside comprises a modified sugar moiety, and wherein at least one of the internucleoside linking groups has Formula XVII:

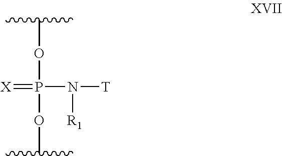

wherein independently for each internucleoside linking group of the modified oligonucleotide having Formula XVII:
X is selected from O or S;
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group; and
R₅ is selected from OCH₃, OH, C₁-C₆ alkyl, and substituted C₁-C₆ alkyl.

Embodiment 706. An antisense agent comprising a modified oligonucleotide consisting of 12-70 linked nucleosides linked through internucleoside linking groups, wherein at least one nucleoside comprises a modified sugar moiety, and wherein at least one of the internucleoside linking groups has Formula XVII:

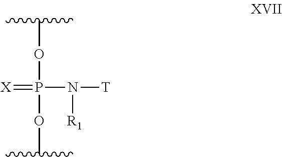

wherein independently for each internucleoside linking group of the modified oligonucleotide having Formula XVII:
   X is selected from O or S;
   R₁ is selected from H, C₁-C₆ alkyl, and substituted C₁-C₆ alkyl; and
   T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group; and
R₅ is selected from OCH₃, OH, C₁-C₆ alkyl, and substituted C₁-C₆ alkyl,
   Provided that if X is 0 and that if R₁ is H, then T is not:

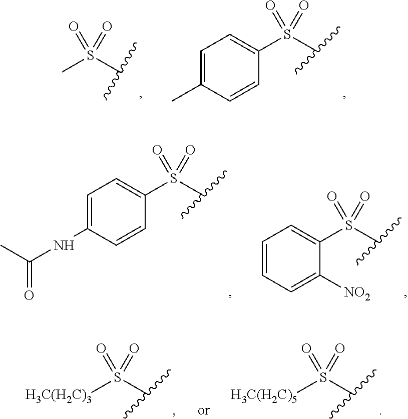

Embodiment 707. The antisense agent of embodiment 705 or embodiment 706, wherein at least one internucleoside linking group is a phosphodiester or a phosphorothioate internucleoside linking group.

Embodiment 708. The antisense agent of any of embodiments 705-707, wherein at least one nucleoside comprises a 2′-β-D-deoxyribosyl sugar moiety.

Embodiment 709. The modified oligonucleotide of any of embodiments 705-708, wherein for at least one internucleoside linking group of Formula XVII, X is O.

Embodiment 710. The modified oligonucleotide of any of embodiments 705-709, wherein for at least one internucleoside linking group of Formula XVII, X is S.

Embodiment 711. The modified oligonucleotide of embodiment 705 or 706, wherein for at least one internucleoside linking group of Formula XVII, R₁ is H.

Embodiment 712. The modified oligonucleotide of embodiment 705 or 706, wherein for at least one internucleoside linking group of Formula XVII, R₁ is a C₁-C₆ alkyl.

Embodiment 713. The modified oligonucleotide of embodiment 710, wherein R₁ is methyl.

Embodiment 714. The modified oligonucleotide of embodiment 705 or 706, wherein for at least one internucleoside linking group of Formula XVII, R₁ is a substituted C₁-C₆ alkyl.

Embodiment 715. The modified oligonucleotide of any of embodiments 705-714, wherein for at least one internucleoside linking group of Formula XVII, T comprises a conjugate group.

Embodiment 716. The modified oligonucleotide of embodiment 715, wherein the conjugate group comprises a cell-targeting moiety.

Embodiment 717. The modified oligonucleotide of embodiment 715, wherein the conjugate group comprises a carbohydrate or carbohydrate cluster.

Embodiment 718. The modified oligonucleotide of any of embodiments 715-717, wherein the conjugate group comprises at least one GalNAc.

Embodiment 719. The modified oligonucleotide of embodiment 715, wherein the conjugate group comprises a C₁₀-C₂₀ alkyl chain.

Embodiment 720. The modified oligonucleotide of embodiment 719, wherein the conjugate group comprises C₁₆ alkyl.

Embodiment 721. The modified oligonucleotide of any of embodiments 705-714, wherein for at least one internucleoside linking group of Formula XVII, T does not comprise a conjugate group.

Embodiment 722. The modified oligonucleotide of any of embodiments 705-714, wherein for at least one internucleoside linking group of Formula XVII, T does not comprise a cell-targeting moiety.

Embodiment 723. The modified oligonucleotide of any of embodiments 705-722, wherein for at least one internucleoside linking group of Formula XVII, T is SO₂R₂.

Embodiment 724. The modified oligonucleotide of embodiment 723, wherein R₂ is an aryl.

Embodiment 725. The modified oligonucleotide of embodiment 723, wherein R₂ is a substituted aryl.

Embodiment 726. The modified oligonucleotide of embodiment 723, wherein R₂ is a heterocycle.

Embodiment 727. The modified oligonucleotide of embodiment 723, wherein R₂ is a substituted heterocycle.

Embodiment 728. The modified oligonucleotide of embodiment 723, wherein R₂ is an aromatic heterocycle.

Embodiment 729. The modified oligonucleotide of embodiment 723, wherein R₂ is a substituted aromatic heterocycle.

Embodiment 730. The modified oligonucleotide of embodiment 723, wherein R₂ is a diazole.

Embodiment 731. The modified oligonucleotide of embodiment 723, wherein R₂ is a substituted diazole.

Embodiment 732. The modified oligonucleotide of embodiment 723, wherein R₂ is an amine.

Embodiment 733. The modified oligonucleotide of embodiment 723, wherein R₂ is a substituted amine.

Embodiment 734. The modified oligonucleotide of embodiment 723, wherein R₂ is a C₁-C₆ alkoxy, C₁-C₆ alkenyl, or C₁-C₆-alkynl.

Embodiment 735. The modified oligonucleotide of embodiment 723, wherein R₂ is C₁-C₂₀, C₁-C₆, C₂-C₂₀, C₂-C₆, or C₁₀-C₂₀ alkyl.

Embodiment 736. The modified oligonucleotide of embodiment 723, wherein R₂ is substituted C₁-C₂₀, C₁-C₆, C₂-C₂₀, C₂-C₆, or C₁₀-C₂₀ alkyl.

Embodiment 737. The modified oligonucleotide of embodiment 723, wherein R₂ comprises a carbohydrate or carbohydrate cluster.

Embodiment 738. The modified oligonucleotide of embodiment 723, wherein R₂ comprises at least one GalNAc.

Embodiment 739. The modified oligonucleotide of embodiment 723, wherein T is:

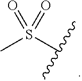 [SMILES: *S(C)(=O)=O]

Embodiment 740. The modified oligonucleotide of embodiment 723, wherein T is:

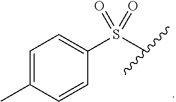 [SMILES: *S(=O)(=O)c1ccc(C)cc1]

Embodiment 741. The modified oligonucleotide of embodiment 723, wherein T is:

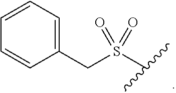 [SMILES: *S(=O)(=O)Cc1ccccc1]

Embodiment 742. The modified oligonucleotide of embodiment 723, wherein T is:

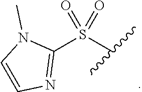 [SMILES: *S(=O)(=O)c1nccn1C]

Embodiment 743. The modified oligonucleotide of embodiment 723, wherein T is:

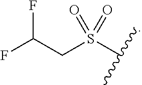 [SMILES: *S(=O)(=O)CC(F)F]

Embodiment 744. The modified oligonucleotide of embodiment 723, wherein T is:

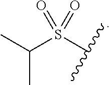 [SMILES: *S(=O)(=O)C(C)C]

Embodiment 745. The modified oligonucleotide of embodiment 723, wherein T is:

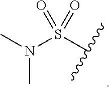 [SMILES: *S(=O)(=O)N(C)C]

Embodiment 746. The modified oligonucleotide of embodiment 723, wherein T is:

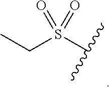 [SMILES: *S(=O)(=O)CC]

Embodiment 747. The modified oligonucleotide of embodiment 723, wherein T is:

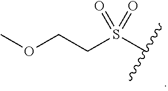 [SMILES: *S(=O)(=O)CCOC]

Embodiment 748. The modified oligonucleotide of embodiment 723, wherein T is:

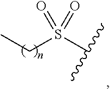

wherein n is from 2 to 20.

Embodiment 749. The modified oligonucleotide of embodiment 748, wherein n is 15.

Embodiment 750. The modified oligonucleotide of any of embodiments 705-722, wherein for at least one internucleoside linking group of Formula XVII, T is C(═O)R₃.

Embodiment 751. The modified oligonucleotide of embodiment 750, wherein R₃ is an aryl.

Embodiment 752. The modified oligonucleotide of embodiment 750, wherein R₃ is a substituted aryl.

Embodiment 753. The modified oligonucleotide of embodiment 750, wherein R₃ is CH₃.

Embodiment 754. The modified oligonucleotide of embodiment 750, wherein R₃ is N(CH₃)2.

Embodiment 755. The modified oligonucleotide of embodiment 750, wherein R₃ is OCH₃.

Embodiment 756. The modified oligonucleotide of embodiment 750, wherein R₃ is a C₁-C₆ alkoxy.

Embodiment 757. The modified oligonucleotide of embodiment 750, wherein R₃ is C₁-C₂₀, C₁-C₆, C₂-C₂₀, C₂-C₆, or C₁₀-C₂₀ alkyl.

Embodiment 758. The modified oligonucleotide of embodiment 750, wherein R₃ is substituted C₁-C₂₀, C₁-C₆, C₂-C₂₀, C₂-C₆, or C₁₀-C₂₀ alkyl.

Embodiment 759. The modified oligonucleotide of embodiment 750, wherein R₃ comprises a carbohydrate or carbohydrate cluster.

Embodiment 760. The modified oligonucleotide of embodiment 750, wherein R₂₃ comprises at least one GalNAc.

Embodiment 761. The modified oligonucleotide of embodiment 750, wherein T is:

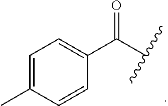 [SMILES: *C(=O)c1ccc(C)cc1]

Embodiment 762. The modified oligonucleotide of embodiment 750, wherein T is:

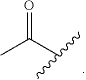 [SMILES: *C(C)=O]

Embodiment 763. The modified oligonucleotide of embodiment 750, wherein T is:

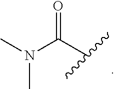 [SMILES: *C(=O)N(C)C]

Embodiment 764. The modified oligonucleotide of embodiment 750, wherein T is:

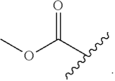 [SMILES: *C(=O)OC]

Embodiment 765. The modified oligonucleotide of embodiment 750, wherein T is:

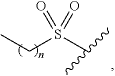

wherein n is from 2 to 20.

Embodiment 766. The modified oligonucleotide of embodiment 765, wherein n is 15.

Embodiment 767. The modified oligonucleotide of any of embodiments 705-722, wherein for at least one internucleoside linking group of Formula XVII, T is P(═O)R₄R₅.

Embodiment 768. The modified oligonucleotide of embodiment 767, wherein R₄ is OCH₃.

Embodiment 769. The modified oligonucleotide of embodiment 767, wherein R₄ is OH.

Embodiment 770. The modified oligonucleotide of embodiment 767, wherein R₄ is C₁-C₆ alkyl.

Embodiment 771. The modified oligonucleotide of embodiment 767, wherein R₄ is substituted C₁-C₆ alkyl.

Embodiment 772. The modified oligonucleotide of embodiment 767, wherein R₄ is C₁-C₂₀, C₁-C₆, C₂-C₂₀, C₂-C₆, or C₁₀-C₂₀ alkyl.

Embodiment 773. The modified oligonucleotide of embodiment 767, wherein R₄ is substituted C₁-C₂₀, C₁-C₆, C₂-C₂₀, C₂-C₆, or C₁₀-C₂₀ alkyl.

Embodiment 774. The modified oligonucleotide of embodiment 767, wherein R₄ comprises a carbohydrate or carbohydrate cluster.

Embodiment 775. The modified oligonucleotide of embodiment 767, wherein R₄ comprises at least one GalNAc.

Embodiment 776. The modified oligonucleotide of any of embodiments 767-775, wherein R₅ is OCH₃.

Embodiment 777. The modified oligonucleotide of any of embodiments 767-775, wherein R₅ is OH.

Embodiment 778. The modified oligonucleotide of any of embodiments 767-775, wherein R₅ is C₁-C₆ alkyl.

Embodiment 779. The modified oligonucleotide of any of embodiments 767-775, wherein R₅ is substituted C₁-C₆ alkyl.

Embodiment 780. The modified oligonucleotide of embodiment 767, wherein T is:

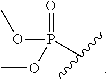 [SMILES: *P(=O)(OC)OC]

Embodiment 781. The modified oligonucleotide of embodiment 767, wherein T is:

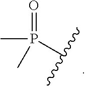 [SMILES: *P(C)(C)=O]

Embodiment 782. The modified oligonucleotide of embodiment 767, wherein T is:

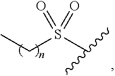

wherein n is from 2 to 20.

Embodiment 783. The modified oligonucleotide of embodiment 782, wherein n is 15.

Embodiment 784. The modified oligonucleotide of any of embodiments 705-783, wherein at least one internucleoside linking group of the modified oligonucleotide is not a linking group of Formula XVII.

Embodiment 785. The modified oligonucleotide of any of embodiments 705-784, wherein exactly one internucleoside linking group of the modified oligonucleotide is an internucleoside linking group of Formula XVII.

Embodiment 786. The modified oligonucleotide of any of embodiments 705-784, wherein exactly two internucleoside linking groups of the modified oligonucleotide are internucleoside linking groups of Formula XVII.

Embodiment 787. The modified oligonucleotide of any of embodiments 705-784, wherein exactly three internucleoside linking groups of the modified oligonucleotide are internucleoside linking groups of Formula XVII.

Embodiment 788. The modified oligonucleotide of any of embodiments 705-784, wherein exactly four internucleoside linking groups of the modified oligonucleotide are internucleoside linking groups of Formula XVII.

Embodiment 789. The modified oligonucleotide of any of embodiments 705-784, wherein exactly five internucleoside linking groups of the modified oligonucleotide are internucleoside linking groups of Formula XVII.

Embodiment 790. The modified oligonucleotide of any of embodiments 705-784, wherein at least six internucleoside linking groups of the modified oligonucleotide are internucleoside linking groups of Formula XVII.

Embodiment 791. The modified oligonucleotide of any of embodiment 705-783 or 785-787 having at least two linking groups of Formula XVII, wherein at least two of the linking groups of Formula XVII are the same as one another.

Embodiment 792. The modified oligonucleotide of any of embodiments 705-791, wherein each internucleoside linking group of the modified oligonucleotide that is not an internucleoside linking group of Formula XVII is either a phosphodiester internucleoside linking group or a phosphorothioate internucleoside linking group.

Embodiment 793. The modified oligonucleotide of any of embodiments 705-784 or 790, wherein each internucleoside linking group of the modified oligonucleotide is an internucleoside linking group of Formula XVII.

Embodiment 794. An antisense agent comprising a modified oligonucleotide, wherein at least one region of the modified oligonucleotide has Structure A:

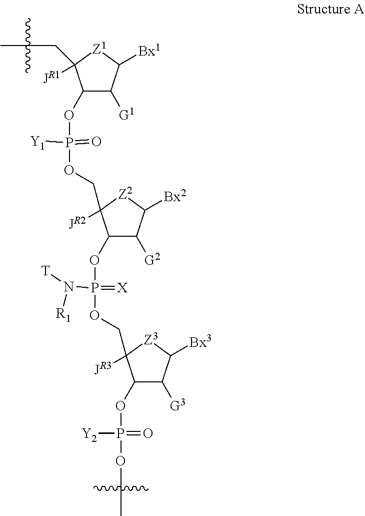

wherein:
each Bx is a heterocyclic base moiety;
X is selected from O or S;
each of Y₁ and Y₂ is independently selected from OH or SH;
each of Z¹, Z², and Z³ are independently selected from —(CH₂)ₚ—X^{Z}—(CH₂)_{q}—, wherein p is 0 or 1, q is 0 or
1, and X^{Z} is O, S, or N(E₁);
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group;
R₅ is selected from OCH₃, OH, C₁-C₆ alkyl, and substituted C₁-C₆ alkyl;
either J^{R1} and G¹ form a J^{R1} to G¹ bridge, or J^{R1} is H and G¹ is selected from H, OH, halogen or O—[C(R₆)(R₂)]ₙ—[(C═O)ₘ—X^{G}]ⱼ—R₈;
either JR₂ and G² form a JR₂ and G² bridge, or JR₂ is H and G² is selected from H, OH, halogen or O—[C(R₆)(R₂)]ₙ[(C═O)ₘ—X^{G}]ⱼ—R₈;
either J^{R3} and G³ form a J^{R3} and G³ bridge, or J^{R3} is H and G³ is selected from H, OH, halogen or O—[C(R₆)(R₇)]ₙ—[(C═O)ₘ—X^{G}]ⱼ—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and
C(=Q₂)N(J₁)(J₂);
Q₂ is O, S or NJ₃; and
each J₁, J₂ and J₃ is, independently, H or C₁-C₆ alkyl.

Embodiment 795. An antisense agent comprising a modified oligonucleotide, wherein at least one region of the modified oligonucleotide has Structure B:

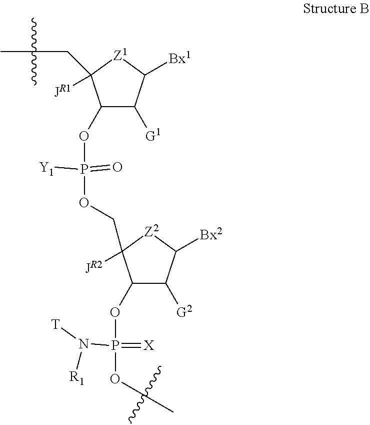

wherein:
   each Bx is a heterocyclic base moiety;
X is selected from O or S;
each of Y₁ and Y₂ is independently selected from OH or SH;
each of Z¹ and Z² are independently selected from —(CH₂)ₚ—X^{Z}—(CH₂)_{q}—, wherein p is 0 or 1, q is 0 or 1, and X^{Z} is O, S, or N(E₁);
R₁ is selected from H, C₁-C₆ alkyl, and substituted C₁-C₆ alkyl; and
T is selected from SO₂R₂, C(═O)R₃, and P(═O)R₄R₅, wherein: R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group;
R₅ is selected from OCH₃, OH, C₁-C₆ alkyl, and substituted C₁-C₆ alkyl;
either J^{R1} and G¹ form a J^{R1} to G¹ bridge, or J^{R1} is H and G¹ is selected from H, OH, halogen or O—[C(R₆)(R₇)]ₙ—[(C═O)ₘ—X^{G}]ⱼ—R₈;
either J^{R2} and G² form a J^{R2} and G² bridge, or J^{R2} is H and G² is selected from H, OH, halogen or O—[C(R₆)(R₇)]ₙ—[(C═O)ₘ—X^{G}]ⱼ—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

Embodiment 796. An antisense agent comprising a modified oligonucleotide, wherein at least one region of the modified oligonucleotide has Structure C:

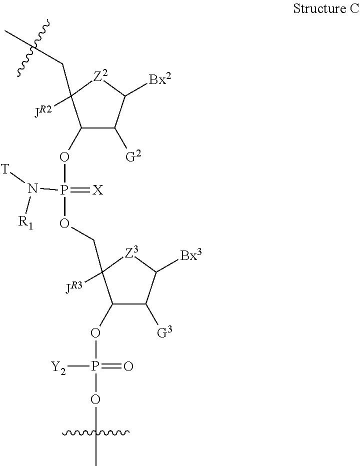

wherein:
each Bx is a heterocyclic base moiety;
X is selected from O or S;
each of Y₁ and Y₂ is independently selected from OH or SH;
each of Z² and Z³ are independently selected from —(CH₂)ₚ—X^{Z}—(CH₂)_{q}—, wherein p is 0 or 1, q is 0 or 1, and X^{Z} is O, S, or N(E₁);
R₁ is selected from H, C₁-C₆ alkyl, and substituted C₁-C₆ alkyl; and
T is selected from SO₂R₂, C(═O)R₃, and P(═O)R₄R₅, wherein: R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group;
R₅ is selected from OCH₃, OH, C₁-C₆ alkyl, and substituted C₁-C₆ alkyl;
   either J^{R2} and G² form a J^{R2} and G² bridge, or J^{R2} is H and G² is selected from H, OH, halogen or O—[C(R₆)(R₇)]ₙ[(C═O)ₘ—X^{G}]—R₈;
either J^{R3} and G³ form a J^{R3} and G³ bridge, or J^{R3} is H and G³ is selected from H, OH, halogen or O—[C(R₆)(R₇)]ₙ—[(C═O)ₘ—X^{G}]ⱼ—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

Embodiment 797. An antisense agent comprising a modified oligonucleotide, wherein at least one region of the modified oligonucleotide has Structure D:

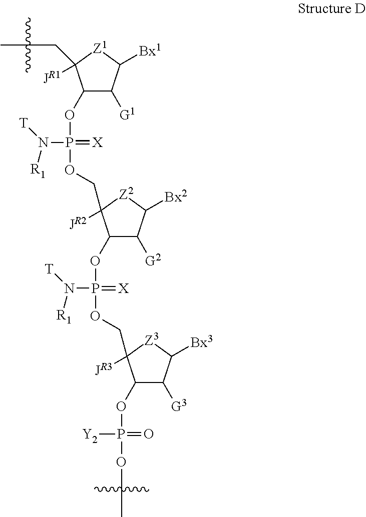

wherein:
each Bx is a heterocyclic base moiety;
X is selected from O or S;
each of Y₁ and Y₂ is independently selected from OH or SH;
each of Z² and Z³ are independently selected from —(CH₂)ₚ—X^{Z}—(CH₂)_{q}—, wherein p is 0 or 1, q is 0 or 1, and X^{Z} is O, S, or N(E₁);
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group;
R₅ is selected from OCH₃, OH, C₁-C₆ alkyl, and substituted C₁-C₆ alkyl;
either J^{R1} and G¹ form a J^{R1} to G¹ bridge, or J^{R1} is H and G¹ is selected from H, OH, halogen or O—[C(R₆)(R₇)]ₙ—[(C═O)ₘ—X^{G}]ⱼ—R₈;
either J^{R2} and G² form a J^{R2} and G² bridge, or J^{R2} is H and G² is selected from H, OH, halogen or O—[C(R₆)(R₇)]ₙ—[(C═O)ₘ—X^{G}]ⱼ—R₈;
either J^{R3} and G³ form a J^{R3} and G³ bridge, or J^{R3} is H and G³ is selected from H, OH, halogen or O—[C(R₆)(R₇)]ₙ—[(C═O)ₘ—X^{G}]ⱼ—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

Embodiment 798. An antisense agent comprising a modified oligonucleotide, wherein at least one region of the modified oligonucleotide has Structure E:

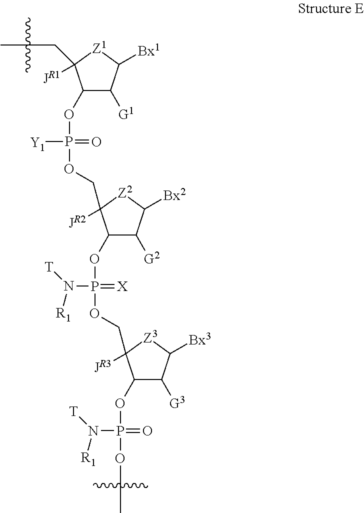

wherein:
each Bx is a heterocyclic base moiety;
X is selected from O or S;
each of Y₁ and Y₂ is independently selected from OH or SH;
each of Z² and Z³ are independently selected from —(CH₂)ₚ—X^{Z}—(CH₂)_{q}—, wherein p is 0 or 1, q is 0 or 1, and X^{Z} is O, S, or N(E₁);
R₁ is selected from H, C₁-C₆ alkyl, and substituted C₁-C₆ alkyl; and
T is selected from SO₂R₂, C(═O)R₃, and P(═O)R₄R₅, wherein: R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group;
R₅ is selected from OCH₃, OH, C₁-C₆ alkyl, and substituted C₁-C₆ alkyl;
either J^{R1} and G¹ form a J^{R1} to G¹ bridge, or J^{R1} is H and G¹ is selected from H, OH, halogen or O—[C(R₆)(R₇)]ₙ[(C═O)ₘ—X^{G}]ⱼ—R₈;
either J^{R}₂ and G² form a J^{R}₂ and G² bridge, or J^{R}₂ is H and G² is selected from H, OH, halogen or O—[C(R₆)(R₇)]ₙ—[(C═O)ₘ—X^{G}]—R₈;
either J^{R3} and G³ form a J^{R3} and G³ bridge, or J^{R3} is H and G³ is selected from H, OH, halogen or O—[C(R₆)(R₇)]ₙ—[(C═O)ₘ—X^{G}]—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

Embodiment 799. The modified oligonucleotide of any of embodiments 794-798, wherein each Z is O.

Embodiment 800. The modified oligonucleotide of any of embodiments 794-799, wherein at least one G is selected from H, OH, halogen, C₁-C₆ alkoxy, —O(CH₂)₂OCH₃, or —OCH₂(C═O)NHCH₃.

Embodiment 801. The modified oligonucleotide of any of embodiments 794-799, wherein each G is selected from H, OH, halogen, C₁-C₆ alkoxy, —O(CH₂)₂OCH₃, or —OCH₂(C═O)NHCH₃.

Embodiment 802. The modified oligonucleotide of any of embodiments 794-801, wherein at least one J^{R} forms a bridge with at least one G, wherein said J^{R} to G bridge has a formula selected from —CH(CH₃)—O— or —(CH₂)ₖ—O′, wherein k is from 1 to 3.

Embodiment 803. The modified oligonucleotide of any of embodiments 794-802, wherein each J^{R} and G form a bridge, wherein said J^{R} to G bridge has a formula selected from —CH(CH₃)—O— or —(CH₂)ₖ—O—, wherein k is from 1 to 3.

Embodiment 804. The modified oligonucleotide of any of embodiments 802 or 803, wherein at least one Z is 0 and the corresponding J^{R} to G bridge has a formula (CH₂)ₖ—O—, wherein k is 1.

Embodiment 805. The modified oligonucleotide of any of embodiments 794-804 wherein each nucleoside of structure A, B, C, D, or E is a stereo standard nucleoside.

Embodiment 806. The modified oligonucleotide of any of embodiments 794-804, wherein at least one nucleoside of structure A, B, C, D, or E is a stereo-non-standard nucleoside.

Embodiment 807. The modified oligonucleotide of any of embodiments 802-804 or 806, wherein at least one nucleoside having a J^{R} to G bridge is in the α-L-ribosyl configuration.

Embodiment 808. The modified oligonucleotide of any of embodiments 794-807, wherein the modified oligonucleotide comprises at least 2, at least 3, at least 4, at least 5, at least 6, at least 7, at least 8, at least 9, or at least 10 regions having structures A, B, C, D, or E.

Embodiment 809. The modified oligonucleotide of any of embodiments 794-807, wherein at least one region having structure A, B, C, D, or E is at the 5′ end of the modified oligonucleotide.

Embodiment 810. The modified oligonucleotide of any of embodiments 794-807, wherein at least one region having structure A, B, C, D, or E is at the 3′ end of the modified oligonucleotide.

Embodiment 811. The modified oligonucleotide of any of embodiments 794-807, wherein at least one region having structure A, B, C, D, or E is internal to the modified oligonucleotide.

Embodiment 812. An antisense agent comprising a modified oligonucleotide consisting of 10-30 linked nucleosides, wherein a region of the modified oligonucleotide has the formula (N_{g1})_{L1}(N_{g2})_{L2}(N_{g3})_{L3}, wherein each N_{g} is a nucleoside and each L is an internucleoside linking group; wherein each of L₁, and L₂ is a phosphodiester internucleoside linking group, a phosphorothioate internucleoside linking group, or an internucleoside linking group of Formula XVII:

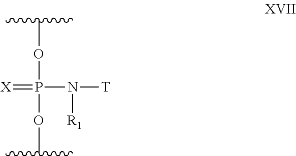

wherein L₃ is absent or is a phosphodiester internucleoside linking group, a phosphorothioate internucleoside linking group, or an internucleoside linking group of Formula XVII;
wherein at least one of L₁, L₂, and L₃ is an internucleoside linking group of Formula XVII; and at least one of L₁, L₂, and L₃ is a phosphorothioate or a phosphodiester internucleoside linking group,
wherein independently for each internucleoside linking group of the modified oligonucleotide having Formula XVII:
   X is selected from O or S;
   R₁ is selected from H, C₁-C₆ alkyl, and substituted C₁-C₆ alkyl; and
   T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
   R₃ is selected from an aryl, a substituted aryl, CH₃, N(CH₃)₂, OCH₃ and a conjugate;
   R₄ is selected from OCH₃, OH, C₁-C₆ alkyl, substituted C₁-C₆ alkyl and a conjugate; and
R₅ is selected from OCH₃, OH, C₁-C₆ alkyl, and substituted C₁-C₆ alkyl.

Embodiment 813. The modified oligonucleotide of embodiment 812, wherein the modified oligonucleotide comprises at least 2, at least 3, at least 4, at least 5, at least 6, at least 7, at least 8, at least 9, or at least 10 regions having the formula (N_{g1})_{L1}(N_{g2})_{L2}(N_{g3})_{L3}.

Embodiment 814. The modified oligonucleotide of embodiment 812 or 813, wherein at least one region having the formula (N_{g1})_{L1}(N_{g2})_{L2}(N_{g3})_{L3} is at the 5′ end of the oligonucleotide

Embodiment 815. The modified oligonucleotide of embodiment 812 or 813, wherein at least one region having the formula (N_{g1})L₁ (N_{g2})_{L2}(N_{g3})_{L3} is internal to the oligonucleotide.

Embodiment 816. The modified oligonucleotide of embodiment 812 or 813, wherein at least one region having the formula (N_{g1})_{L1}(N_{g2})_{L2}(N_{g3})_{L3} is at the 3′ end of the oligonucleotide.

Embodiment 817. The modified oligonucleotide of any of embodiments 705-816, wherein at least one nucleoside of the modified oligonucleotide is a modified nucleoside selected from a bicyclic nucleoside and a non-bicyclic substituted nucleoside.

Embodiment 818. The modified oligonucleotide of any of embodiments 705-817, wherein at least one nucleoside of the modified oligonucleotide is selected from: a β-D-LNA nucleoside, an α-L-LNA nucleoside, an ENA nucleoside, a cEt nucleoside, a 2′-MOE nucleoside, a 2′-OMe nucleoside, a 2′-F nucleoside, a 2′-NMA nucleoside, a 5′-Me nucleoside, a DNA nucleoside, and an RNA nucleoside.

Embodiment 819. The modified oligonucleotide of any of embodiments 705-818, wherein each nucleoside of the modified oligonucleotide is selected from: a β-D-LNA nucleoside, an α-L-LNA nucleoside, an ENA nucleoside, a cEt nucleoside, a 2′-MOE nucleoside, a 2′-OMe nucleoside, a 2′-F nucleoside, a 2′-NMA nucleoside, a 5′-Me nucleoside, a DNA nucleoside, and an RNA nucleoside.

Embodiment 820. The modified oligonucleotide of any of embodiments 705-819, wherein at least one nucleoside of the modified oligonucleotide is a stereo-non-standard nucleoside.

Embodiment 821. The modified oligonucleotide of embodiment 820, wherein the internucleoside linking group linking at least one stereo-non-standard nucleoside to an adjacent nucleoside is an internucleoside linking group of Formula XVII.

Embodiment 822. The modified oligonucleotide of embodiment 820 or 821, wherein at least two nucleosides of the modified oligonucleotide are stereo-non-standard nucleosides.

Embodiment 823. The modified oligonucleotide of embodiment 822, wherein at least two stereo-non-standard nucleosides of the modified oligonucleotide are adjacent to one another.

Embodiment 824. The modified oligonucleotide of embodiment 823, wherein at least two stereo-non-standard nucleosides of the modified oligonucleotide are linked to one another with an internucleoside linking group of Formula XVII.

Embodiment 825. The modified oligonucleotide of any of embodiments 820-824, wherein at least one stereo-non-standard nucleoside of the modified oligonucleotide is a stereo-non-standard DNA nucleoside.

Embodiment 826. The modified oligonucleotide of embodiment 825 wherein the stereo-non-standard DNA nucleoside is selected from a stereo-non-standard DNA nucleoside having: Formula I, Formula II, Formula III, Formula IV, Formula V, Formula VI, and Formula VII.

Embodiment 827. The modified oligonucleotide of embodiment 826 wherein the stereo-non-standard DNA nucleoside is selected from a stereo-non-standard DNA nucleoside having: Formula V and Formula II.

Embodiment 828. The modified oligonucleotide of any of embodiments 820-827, wherein at least one stereo-non-standard nucleoside of the modified oligonucleotide is a substituted stereo-non-standard nucleoside or a stereo-non-standard RNA nucleoside.

Embodiment 829. The modified oligonucleotide of embodiment 828, wherein the 2′-substituent of the at least one substituted stereo-non-standard nucleoside of the modified oligonucleotide is selected from: 2′-MOE, 2′-OMe, 2′-F, or 2′-OH.

Embodiment 830. The modified oligonucleotide of any of embodiments 705-819, wherein each nucleoside is a stereo-standard nucleoside.

Embodiment 831. The modified oligonucleotide of any of embodiments 705-829, wherein the modified oligonucleotide consists of 12-30 linked nucleosides.

Embodiment 832. The modified oligonucleotide of any of embodiments 705-829, wherein the modified oligonucleotide consists of 16-24 linked nucleosides.

Embodiment 833. The modified oligonucleotide of any of embodiments 705-829, wherein the modified oligonucleotide consists of 18-22 linked nucleosides.

Embodiment 834. The modified oligonucleotide of any of embodiments 705-832, wherein the modified oligonucleotide consists of 16 linked nucleosides.

Embodiment 835. The modified oligonucleotide of any of embodiments 705-832, wherein the modified oligonucleotide consists of 17 linked nucleosides.

Embodiment 836. The modified oligonucleotide of any of embodiments 705-833, wherein the modified oligonucleotide consists of 18 linked nucleosides.

Embodiment 837. The modified oligonucleotide of any of embodiments 705-833, wherein the modified oligonucleotide consists of 19 linked nucleosides.

Embodiment 838. The modified oligonucleotide of any of embodiments 705-833, wherein the modified oligonucleotide consists of 20 linked nucleosides.

Embodiment 839. The modified oligonucleotide of any of embodiments 705-833, wherein the modified oligonucleotide consists of 21 linked nucleosides.

Embodiment 840. The modified oligonucleotide of any of embodiments 705-833, wherein the modified oligonucleotide consists of 22 linked nucleosides.

Embodiment 841. The modified oligonucleotide of any of embodiments 705-832, wherein the modified oligonucleotide consists of 23 linked nucleosides.

Embodiment 842. The modified oligonucleotide of any of embodiments 705-841, wherein at least one nucleoside of the modified oligonucleotide is selected from: a 2′-OMe nucleoside, a 2′-F nucleoside, and an RNA nucleoside.

Embodiment 843. The modified oligonucleotide of any of embodiments 705-842, wherein at least one nucleoside of the modified oligonucleotide is a 2′-OMe nucleoside, and at least one nucleoside of the modified oligonucleotide is a 2′-F nucleoside.

Embodiment 844. The modified oligonucleotide of embodiment 843, wherein each nucleoside of the modified oligonucleotide is selected from a 2′-OMe nucleoside or a 2′-F nucleoside.

Embodiment 845. The modified oligonucleotide of any of embodiments 705-743, wherein at least one nucleoside of the modified oligonucleotide is a 2′-OMe nucleoside, at least one nucleoside of the modified oligonucleotide is a 2′-F nucleoside, and at least one nucleoside of the modified oligonucleotide comprises a sugar surrogate.

Embodiment 846. The modified oligonucleotide of embodiment 845, wherein each nucleoside of the modified oligonucleotide is selected from a 2′-OMe nucleoside, a 2′-F nucleoside, and a nucleoside comprising a sugar surrogate.

Embodiment 847. The modified oligonucleotide of any of embodiments 845-846, wherein the nucleoside comprising a sugar surrogate is selected from:

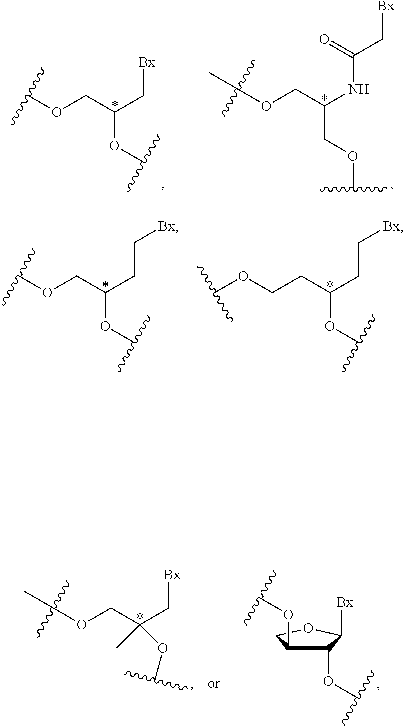

wherein Bx is a heterocyclic base moiety.

Embodiment 848. The modified oligonucleotide of embodiment 847, wherein the nucleoside comprising a sugar surrogate is GNA.

Embodiment 849. The modified oligonucleotide of any of embodiments 842-848, wherein the modified oligonucleotide has a region of alternating nucleoside types having the motif ABABA, wherein each A is a stereo-standard nucleoside of a first type and each B is a stereo-standard nucleoside of a second type, wherein the first type and the second type are different from one another.

Embodiment 850. The modified oligonucleotide of embodiment 849, wherein A and B are selected from 2′-F substituted nucleosides, 2′-OMe substituted nucleosides, and stereo-standard RNA nucleosides.

Embodiment 851. The modified oligonucleotide of any of embodiments 705-850, wherein the 5′-end of the modified oligonucleotide comprises a terminal group.

Embodiment 852. The modified oligonucleotide of embodiment 851, wherein the terminal group is a stabilized phosphate group.

Embodiment 853. The modified oligonucleotide of embodiment 852, wherein the stabilized phosphate group is a 5′-vinyl phosphonate or a 5′-cyclopropyl phosphonate.

Embodiment 854. The modified oligonucleotide of embodiment 851, wherein the terminal group is selected from

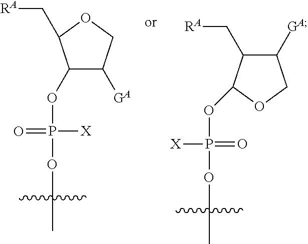

wherein R^{A} is OH, OP(═O)OH, OP(═O)SH, or a stabilized phosphate group;
G^{A} is H, OH, OMe, MOE, or a halogen;
X is OH, SH, or NSO₂R₂;
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group.

Embodiment 855. The modified oligonucleotide of embodiment 854, wherein G^{A} is selected from H or OH and X is SH.

Embodiment 856. The antisense agent of any of embodiments 705-855, wherein the antisense agent is an RNAi agent.

Embodiment 857. The RNAi agent of embodiment 856, wherein the RNAi agent is a single-stranded RNAi agent comprising an RNAi antisense modified oligonucleotide, wherein the RNAi antisense modified oligonucleotide is a modified oligonucleotide of any of embodiments 705-855.

Embodiment 858. The RNAi agent of embodiment 856, wherein the RNAi agent is an oligonucleotide duplex comprising an RNAi antisense modified oligonucleotide and an RNAi sense modified oligonucleotide, wherein the RNAi antisense modified oligonucleotide and/or the RNAi sense modified oligonucleotide is a modified oligonucleotide of any of embodiments 705-855.

Embodiment 859. The RNAi agent of embodiment 857 or 858, wherein at least one internucleoside linking group of the RNAi antisense modified oligonucleotide is an internucleoside linking group of Formula XVII.

Embodiment 860. The RNAi agent of embodiment 857 or 858, wherein at least two internucleoside linking groups of the RNAi antisense modified oligonucleotide are independently selected internucleoside linking groups of Formula XVII.

Embodiment 861. The RNAi agent of any of embodiments 857-860, wherein at least one of the five 3′-most internucleoside linking groups of the RNAi antisense modified oligonucleotide is an internucleoside linking group of Formula XVII.

Embodiment 862. The RNAi agent of any of embodiments 857-861, wherein at least two of the five 3′-most internucleoside linking groups of RNAi antisense modified oligonucleotide is an internucleoside linking group of Formula XVII.

Embodiment 863. The RNAi agent of any of embodiments 857-862, wherein 1-3 of the three 3′-most internucleoside linking groups are internucleoside linking groups of Formula XVII, and each of these three internucleoside linking groups that is not an internucleoside linking group of Formula XVII is a phosphodiester or phosphorothioate internucleoside linking group.

Embodiment 864. The RNAi agent of embodiment 863, wherein the two 3′-most internucleoside linking groups are internucleoside linking groups of Formula XVII.

Embodiment 865. The RNAi agent of any of embodiments 857-864, wherein exactly one of the 5′-most and penultimate 5′-most internucleoside linking groups is an internucleoside linking group of Formula XVII.

Embodiment 866. The RNAi agent of any of embodiments 857-865, wherein exactly one of the 5′-most and penultimate 5′-most internucleoside linking groups of the RNAi antisense oligonucleotide is an internucleoside linking groups of Formula XVII, the other of the 5′-most and penultimate 5′-most internucleoside linking groups of the RNAi antisense oligonucleotide is selected from a phosphodiester and a phosphorothioate internucleoside linkage, the two 3′-most internucleoside linking groups of the RNAi antisense oligonucleotide are internucleoside linking groups of Formula XVII, and the remaining internucleoside linking groups of the RNAi antisense oligonucleotide are phosphodiester internucleoside linkages.

Embodiment 867. The RNAi agent of any of embodiments 857-866, wherein the antisense modified oligonucleotide comprises a 3′-overhang.

Embodiment 868. The RNAi agent of embodiment 867, wherein the 3′-overhang consists of two nucleosides.

Embodiment 869. The RNAi agent of any of embodiments 857-865 or 867-868, wherein at least one internucleoside linking group within the seed region of the RNAi antisense modified oligonucleotide is an intemucleoside linking group of Formula XVII.

Embodiment 870. The RNAi agent of any of embodiments 857-869, wherein for each intemucleoside linking group of Formula XVII, R₁ is H and T is SO₂Me.

Embodiment 871. The RNAi agent of any of embodiments 857-870, wherein the RNAi antisense modified oligonucleotide consists of 23 linked nucleosides, and the internucleoside linkage motif is selected from: ooooooooooooooooooooaa, aaoooooooooooooooooooo, aaooooooooooooooooooaa, asooooooooooooooooooss, saoooooooooooooooooooo, oooooooooooooooooooaaa, ooooooooooooooooaaaoss, oooooooooooooaaaooooss, ooooooooooaaaoooooooss, oooooooaaaooooooooooss, ooooaaaoooooooooooooss, saoooaoooooooaoaooooss, ssoooaoooooooaoaooooss, or ssooooooooooooooooooaa, wherein each “a” represents an internucleoside linkage of Formula XVII, each “s” represents a phosphorothioate intemucleoside linkage, and each “o” represents a phosphodiester internucleoside linkage.

Embodiment 872. The RNAi agent of embodiment 871, wherein the internucleoside linkage motif of the RNAi antisense modified oligonucleotide is selected from oooooooooooooooooooaa, asooooooooooooooooooss, or saoooooooooooooooooooo.

Embodiment 873. The RNAi agent of embodiment 871 or 872, wherein the sugar motif of the RNAi antisense modified oligonucleotide from 5′ to 3′ is yfyfyfyfyfyfyfyfyfyfyfy or yfyyyfyyyyyyyfyfyyyyyyy, wherein “y” represents a 2′-OMe sugar moiety and “f” represents a 2′-F sugar moiety.

Embodiment 874. The RNAi agent of any of embodiments 857-870, wherein the RNAi antisense modified oligonucleotide consists of 21 linked nucleosides, and the internucleoside linkage motif is selected from: aaososososososssssss, ssaaosososososssssss, ssosaaososososssssss, ssososaaosososssssss, ssosososaaososssssss, ssososososaaosssssss, ssosososososaassssss, ssososososososaassss, ssososososososssaass, ssososososososssssaa wherein each “a” represents an internucleoside linkage of Formula XVII, each “s” represents a phosphorothioate internucleoside linkage, and each “o” represents a phosphodiester internucleoside linkage.

Embodiment 875. The RNAi agent of embodiment 874, wherein the internucleoside linkage motif of the RNAi antisense modified oligonucleotide is selected from aaososososososssssss, ssaaosososososssssss, ssososaaosososssssss, ssosososaaososssssss, ssososososososssaass, or ssososososososssssaa, wherein each “a” represents an internucleoside linkage of Formula XVII, each “s” represents a phosphorothioate internucleoside linkage, and each “o” represents a phosphodiester internucleoside linkage.

Embodiment 876. The RNAi agent of embodiment 874 or 875, wherein the sugar motif of the RNAi antisense modified oligonucleotide from 5′ to 3′ is yfyfyfyfyfyfyfyfyfyfyfy, wherein “y” represents a 2′-OMe sugar moiety and “f” represents a 2′-F sugar moiety.

Embodiment 877. The RNAi agent of any of embodiments 873-876 wherein each “a” is a mesyl phosphoramidate linkage.

Embodiment 878. The RNAi agent of any of embodiments 857-878, wherein at least one region of the RNAi antisense modified oligonucleotide has structure A, B, C, D, or E.

Embodiment 879. The RNAi agent of embodiment 878, wherein at least one region having structure A, B, C, D, or E is within the seed region of the RNAi antisense modified oligonucleotide.

Embodiment 880. The RNAi agent of embodiment 878, wherein at least one region having structure A, B, C, D, or E is at the 3′ end of the RNAi antisense modified oligonucleotide.

Embodiment 881. The RNAi agent of any of embodiments 857-880, wherein at least one region of the RNAi antisense modified oligonucleotide has the formula (N_{g1})_{L1}(N_{g2})_{L2}(N_{g3})_{L3}, wherein each N_{g} is a nucleoside and each L is an internucleoside linking group; wherein each of L₁, and L₂ is a phosphodiester internucleoside linking group, a phosphorothioate internucleoside linking group, or an internucleoside linking group of Formula XVII:

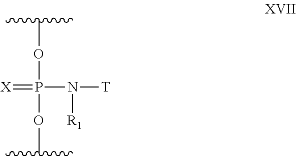

wherein L₃ is absent or is a phosphodiester internucleoside linking group, a phosphorothioate internucleoside linking group, or an internucleoside linking group of Formula XVII;
wherein at least one of L₁, L₂, and L₃ an internucleoside linking group of Formula XVII; and at least one of L₁, L₂, and L₃ is a phosphorothioate or a phosphodiester internucleoside linking group,
wherein independently for each internucleoside linking group of the modified oligonucleotide having Formula XVII:
   X is selected from O or S;
   R₁ is selected from H, C₁-C₆ alkyl, and substituted C₁-C₆ alkyl; and
   T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, substituted C₁-C₆ alkyl, and a conjugate;
   R₃ is selected from an aryl, a substituted aryl, CH₃, N(CH₃)₂, OCH₃ and a conjugate;
   R₄ is selected from OCH₃, OH, C₁-C₆ alkyl, substituted C₁-C₆ alkyl and a conjugate; and
   R₅ is selected from OCH₃, OH, C₁-C₆ alkyl, and substituted C₁-C₆ alkyl.

Embodiment 882. The RNAi agent of embodiment 881, wherein the region having the formula (N_{g1})_{L1}(N_{g2})_{L2}(N_{g3})_{L3} includes one or two 3′-overhang nucleosides.

Embodiment 883. The RNAi agent of embodiment 881, wherein at least one region having the formula (N_{g1})_{L1}(N_{g2})_{L2}(N_{g3})_{L3} is at the 3′ end of the RNAi antisense modified oligonucleotide.

Embodiment 884. The RNAi agent of embodiment 883, wherein L1 and L2 are each internucleoside linkages of Formula XVII wherein R₁ is H and T is SO₂Me, and L3 is a phosphodiester internucleoside linkage.

Embodiment 885. The RNAi agent of embodiment 881, wherein at least one region having the formula (N_{g1})_{L1}(N_{g2})_{L2}(N_{g3})_{L3} is at the 5′ end of the RNAi antisense modified oligonucleotide.

Embodiment 886. The RNAi agent of embodiment 885, wherein one of L 1 or L2 is an internucleoside linkages of Formula XVII wherein R₁ is H and T is SO₂Me, the other of L1 or L2 is a phosphorothioate internucleoside linkage, and L3 is a phosphodiester internucleoside linkage.

Embodiment 887. The RNAi agent of embodiment 881, wherein at least one region having the formula (N_{g1})_{L1}(N_{g2})_{L2}(N_{g3})_{L3} is within the seed region of the RNAi antisense modified oligonucleotide.

Embodiment 888. The RNAi agent of any of embodiments 857-887, wherein the region of the RNAi antisense oligonucleotide that is complementary to a target is at least 15 nucleobases.

Embodiment 889. The RNAi agent of any of embodiments 857-888, wherein the region of the RNAi antisense oligonucleotide that is complementary to a target is at least 17 nucleobases.

Embodiment 890. The RNAi agent of any of embodiments 857-889, wherein the region of the RNAi antisense oligonucleotide that is complementary to a target is at least 19 nucleobases.

Embodiment 891. The RNAi agent of any of embodiments 857-890, wherein the region of the RNAi antisense oligonucleotide that is complementary to a target is at least 21 nucleobases.

Embodiment 892. The RNAi agent of any of embodiments 857-890, wherein the region of the RNAi antisense oligonucleotide that is complementary to a target is exactly 19 nucleobases.

Embodiment 893. The RNAi agent of any of embodiments 857-891, wherein the region of the RNAi antisense oligonucleotide that is complementary to a target is exactly 21 nucleobases.

Embodiment 894. The RNAi agent of any of embodiments 857-893, wherein at least one nucleoside of the RNAi antisense modified oligonucleotide is selected from: a 2′-OMe nucleoside, a 2′-F nucleoside, and an RNA nucleoside.

Embodiment 895. The RNAi agent of any of embodiments 857-894, wherein at least one nucleoside of the modified oligonucleotide is a 2′-OMe nucleoside, and at least one nucleoside of the modified oligonucleotide is an RNA nucleoside.

Embodiment 896. The RNAi agent of any of embodiments 857-894, wherein at least one nucleoside of the RNAi antisense modified oligonucleotide is a 2′-OMe nucleoside, and at least one nucleoside of the RNAi antisense modified oligonucleotide is a 2′-F nucleoside.

Embodiment 897. The RNAi agent of embodiment 896, wherein each nucleoside of the RNAi antisense modified oligonucleotide is selected from a 2′-OMe nucleoside or a 2′-F nucleoside.

Embodiment 898. The RNAi agent of any of embodiments 887-894, wherein at least one nucleoside of the RNAi antisense modified oligonucleotide is a 2′-OMe nucleoside, at least one nucleoside of the RNAi antisense modified oligonucleotide is a 2′-F nucleoside, and at least one nucleoside of the modified oligonucleotide comprises a sugar surrogate.

Embodiment 899. The RNAi agent of embodiment 898, wherein each nucleoside of the RNAi antisense modified oligonucleotide is selected from a 2′-OMe nucleoside, a 2′-F nucleoside, and a nucleoside comprising a sugar surrogate.

Embodiment 900. The RNAi agent of any of embodiments 898-899, wherein the nucleoside comprising a sugar surrogate is selected from:

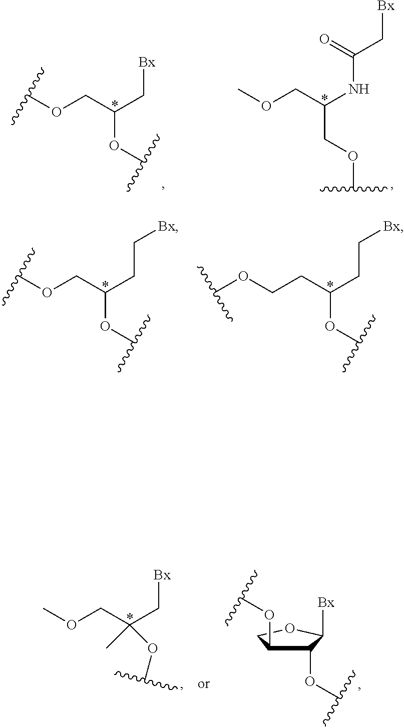

wherein Bx is a heterocyclic base moiety.

Embodiment 901. The RNAi agent of embodiment 900, wherein the nucleoside comprising a sugar surrogate is GNA.

Embodiment 902. The RNAi agent of embodiment 900 or 901, wherein at least one nucleoside comprising a sugar surrogate is one of the nine 5′-most nucleosides of the RNAi antisense modified oligonucleotide.

Embodiment 903. The RNAi agent of any of embodiments 857-902, wherein the modified oligonucleotide has a region of alternating nucleoside types having the motif ABABA, wherein each A is a stereo-standard nucleoside of a first type and each B is a stereo-standard nucleoside of a second type, wherein the first type and the second type are different from one another.

Embodiment 904. The RNAi agent of embodiment 903, wherein A and B are selected from 2′-F substituted nucleosides, 2′-OMe substituted nucleosides, and stereo-standard RNA nucleosides.

Embodiment 905. The RNAi agent of any of embodiments 857-904, wherein the 5′-end of the RNAi antisense modified oligonucleotide comprises a terminal group.

Embodiment 906. The RNAi agent of embodiment 905, wherein the terminal group is a stabilized phosphate group.

Embodiment 907. The RNAi agent of embodiment 906, wherein the stabilized phosphate group is a 5′-vinyl phosphonate or a 5′-cyclopropyl phosphonate.

Embodiment 908. The RNAi agent of embodiment 905, wherein the terminal group is selected from

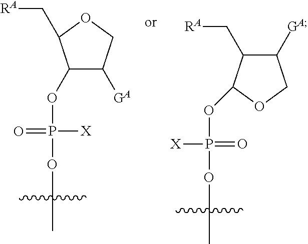

Wherein R^{A} is OH, OP(═O)OH, OP(═O)SH or a stabilized phosphate group;
G^{A} is H, OH, OMe, MOE, or a halogen;
X is OH, SH, or NSO₂R₂;
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group.

Embodiment 909. The RNAi agent of embodiment 908, wherein G^{A} is selected from H or OH and X is SH.

Embodiment 910. The RNAi agent of any of embodiments 858-909, wherein at least one internucleoside linking group of the RNAi sense modified oligonucleotide is an internucleoside linking group of Formula XVII.

Embodiment 911. The RNAi agent of embodiment 910, wherein at least one of the five 5′-most internucleoside linking groups of the RNAi sense modified oligonucleotide is an internucleoside linking group of Formula XVII.

Embodiment 912. The RNAi agent of embodiment 910, wherein at least two of the five 5′-most internucleoside linking groups of the RNAi sense modified oligonucleotide are internucleoside linking groups of Formula XVII.

Embodiment 913. The RNAi agent of embodiment 910, wherein the two 5′-most internucleoside linking groups of the RNAi sense modified oligonucleotide are internucleoside linking groups of Formula XVII.

Embodiment 914. The RNAi agent of any of embodiments 910-913, wherein at least one of the five 3′-most internucleoside linking groups of the RNAi sense modified oligonucleotide is an internucleoside linking group of Formula XVII.

Embodiment 915. The RNAi agent of any of embodiments 910-913, wherein at least two of the five 3′-most internucleoside linking groups of RNAi sense modified oligonucleotide is an internucleoside linking group of Formula XVII.

Embodiment 916. The RNAi agent of any of embodiments 910-913, wherein the two 3′-most internucleoside linking groups are internucleoside linking groups of Formula XVII.

Embodiment 917. The RNAi agent of embodiment 910, wherein the two 3′-most and the two 5′-most internucleoside linking groups of the RNAi sense oligonucleotide are internucleoside linking groups of Formula XVII, and the remaining internucleoside linking groups of the RNAi sense oligonucleotide are phosphodiester internucleoside linkages.

Embodiment 918. The RNAi agent of any of embodiments 910-917, wherein for each internucleoside linking group of Formula XVII, R₁ is H and T is SO₂Me.

Embodiment 919. The RNAi agent of any of embodiments 910-918, wherein the RNAi sense modified oligonucleotide consists of 21 linked nucleosides, and the internucleoside linkage motif is selected from: ooooooooooooooooooaa, aaooooooooooooooooaa, ooooooooooooooooooaa, or ssooooaoaaaooooooooo, wherein each “a” represents an internucleoside linkage of Formula XVII, each “s” represents a phosphorothioate internucleoside linkage, and each “o” represents a phosphodiester internucleoside linkage.

Embodiment 920. The RNAi agent of embodiment 919, wherein the internucleoside linkage motif of the RNAi sense modified oligonucleotide is selected from ooooooooooooooooooaa, aaooooooooooooooooaa, or ooooooooooooooooooaa, wherein each “a” represents an internucleoside linkage of Formula XVII, each “s” represents a phosphorothioate internucleoside linkage, and each “o” represents a phosphodiester internucleoside linkage.

Embodiment 921. The RNAi agent of embodiment 919 or 920, wherein the sugar motif of the RNAi sense modified oligonucleotide is selected from: yyyyyyfyfffyyyyyyyyyy or fyfyfyfyfyfyfyfyfyfyf, wherein “y” represents a 2′-OMe sugar moiety and “f” represents a 2′-F sugar moiety.

Embodiment 922. The RNAi agent of embodiment 921, wherein the RNAi sense modified oligonucleotide has an internucleoside linkage motif of aaooooooooooooooooaa wherein each “a” represents an internucleoside linkage of Formula XVII, each “s” represents a phosphorothioate internucleoside linkage, and each “o” represents a phosphodiester internucleoside linkage, and a sugar motif of yyyyyyfyfffyyyyyyyyyy, wherein “y” represents a 2′-OMe sugar moiety and “f” represents a 2′-F sugar moiety.

Embodiment 923. The RNAi agent of any of embodiments 919-922 wherein each “a” is a mesyl phosphoramidate linkage.

Embodiment 924. The RNAi agent of any of embodiments 910-923, wherein at least one region of the RNAi sense modified oligonucleotide has structure A, B, C, D, or E.

Embodiment 925. The RNAi agent of embodiment 924, wherein at least one region having structure A, B, C, D, or E is at the 3′ end of the RNAi sense modified oligonucleotide.

Embodiment 926. The RNAi agent of embodiment 924, wherein at least one region having structure A, B, C, D, or E is at the 5′ end of the RNAi sense modified oligonucleotide.

Embodiment 927. The RNAi agent of any of embodiments 910-926, wherein at least one region of the RNAi sense modified oligonucleotide has the formula (N_{g1})_{L1}(N_{g2})_{L2}(N_{g3})_{L3}, wherein each N_{g} is a nucleoside and each L is an internucleoside linking group; wherein each of L₁, and L₂ is a phosphodiester internucleoside linking group, a phosphorothioate internucleoside linking group, or an internucleoside linking group of Formula XVII:

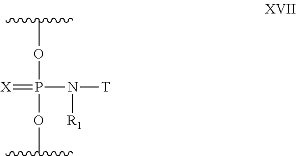

wherein L₃ is absent or is a phosphodiester internucleoside linking group, a phosphorothioate internucleoside linking group, or an internucleoside linking group of Formula XVII;
wherein at least one of L₁, L₂, and L₃ an internucleoside linking group of Formula XVII; and at least one of L₁, L₂, and L₃ is a phosphorothioate or a phosphodiester internucleoside linking group,
   wherein independently for each internucleoside linking group of the modified oligonucleotide having Formula XVII:
   X is selected from O or S;
   R₁ is selected from H, C₁-C₆ alkyl, and substituted C₁-C₆ alkyl; and
   T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
   R₃ is selected from an aryl, a substituted aryl, CH₃, N(CH₃)₂, OCH₃ and a conjugate;
   R₄ is selected from OCH₃, OH, C₁-C₆ alkyl, substituted C₁-C₆ alkyl and a conjugate; and
   R₅ is selected from OCH₃, OH, C₁-C₆ alkyl, and substituted C₁-C₆ alkyl.

Embodiment 928. The RNAi agent of embodiment 927, wherein at least one region having the formula (N_{g1})_{L1}(N_{g2})_{L2}(N_{g3})_{L3} is at the 3′ end of the RNAi sense modified oligonucleotide.

Embodiment 929. The RNAi agent of embodiment 928, wherein L 1 and L2 are internucleoside linking groups of Formula XVII, wherein R₁ is H and T is SO₂Me, and L3 is a phosphodiester internucleoside linkage.

Embodiment 930. The RNAi agent of embodiment 927, wherein at least one region having the formula (N_{g1})_{L1}(N_{g2})_{L2}(N_{g3})_{L3} is at the 5′ end of the RNAi sense modified oligonucleotide.

Embodiment 931. The RNAi agent of embodiment 930, wherein L1 is a phosphodiester internucleoside linking group and L2 and L3 are each internucleoside linking groups of Formula XVII, wherein R₁ is H and T is SO₂Me.

Embodiment 932. The RNAi agent of any of embodiments 858-931, wherein the RNAi sense modified oligonucleotide comprises a 3′ terminal group and/or a 5′ terminal group.

Embodiment 933. The RNAi agent of any of embodiments 858-932, wherein the RNAi sense strand comprises a conjugate group.

Embodiment 934. The RNAi agent of embodiment 933, wherein the conjugate group comprises a cell-targeting moiety.

Embodiment 935. The RNAi agent of embodiment 933, wherein the conjugate group comprises a carbohydrate or carbohydrate cluster.

Embodiment 936. The RNAi agent of embodiment 933, wherein the conjugate group comprises at least one GalNAc.

Embodiment 937. The RNAi agent of embodiment 933, wherein the conjugate group comprises a C₁₀-C₂₀ alkyl chain.

Embodiment 938. The RNAi agent of embodiment 933, wherein the conjugate group comprises C₁₆ alkyl.

Embodiment 939. The RNAi agent of any of embodiments 858-938, wherein the double-stranded region of the oligonucleotide duplex is at least 15 nucleosides.

Embodiment 940. The RNAi agent of any of embodiments 858-938, wherein the double-stranded region of the oligonucleotide duplex is at least 17 nucleosides.

Embodiment 941. The RNAi agent of any of embodiments 858-938, wherein the double-stranded region of the oligonucleotide duplex is at least 19 nucleosides.

Embodiment 942. The RNAi agent of any of embodiments 858-938, wherein the double-stranded region of the oligonucleotide duplex is exactly 19 nucleosides.

Embodiment 943. The modified oligonucleotide of any of embodiments 705-841, wherein each nucleoside of the modified oligonucleotide is a modified nucleoside comprising a modified sugar moiety.

Embodiment 944. The modified oligonucleotide of embodiment 943, wherein each modified sugar moiety is independently selected from a bicyclic sugar moiety and a 2′-substituted furanosyl sugar moiety.

Embodiment 945. The modified oligonucleotide of embodiment 943 or 944, wherein each modified sugar moiety comprises the same modification.

Embodiment 946. The modified oligonucleotide of any of embodiments 943-945, wherein each modified sugar moiety is selected from a 2′-OMe sugar moiety, a 2′-MOE sugar moiety, and a 2′-NMA sugar moiety.

Embodiment 947. The modified oligonucleotide of embodiment 943 or 944, wherein the three 3′-most nucleosides comprise a bicyclic sugar moiety, and the remaining nucleosides comprise a 2′-substituted furanosyl sugar moiety.

Embodiment 948. The modified oligonucleotide of embodiment 943 or 944, wherein the four 3′-most nucleosides comprise a bicyclic sugar moiety, and the remaining nucleosides comprise a 2′-substituted furanosyl sugar moiety.

Embodiment 949. The modified oligonucleotide of embodiment 943 or 944, wherein the five 3′-most nucleosides comprise a bicyclic sugar moiety, and the remaining nucleosides comprise a 2′-substituted furanosyl sugar moiety.

Embodiment 950. The modified oligonucleotide of embodiment 943 or 944, wherein the six 3′-most nucleosides comprise a bicyclic sugar moiety, and the remaining nucleosides comprise a 2′-substituted furanosyl sugar moiety.

Embodiment 951. The modified oligonucleotide of any of embodiments 947-950, wherein each bicyclic sugar moiety is selected from among cEt, LNA, and ENA.

Embodiment 952. The modified oligonucleotide of embodiment 951, wherein the bicyclic sugar moiety is cEt.

Embodiment 953. The modified oligonucleotide of any of embodiments 947-952, wherein the 2′-substituted furanosyl sugar moiety is selected from 2′-OMe, 2′-MOE, and 2′-F.

Embodiment 954. The modified oligonucleotide of any of embodiments 943-953, wherein at least one of the ten 5′-most linking groups of the modified oligonucleotide is an internucleoside linking group of Formula XVII.

Embodiment 955. The modified oligonucleotide of embodiment 954, wherein at least 2 of the ten 5′-most linking groups of the modified oligonucleotide are internucleoside linking groups of Formula XVII.

Embodiment 956. The modified oligonucleotide of embodiment 954, wherein at least 3 of the ten 5′-most linking groups of the modified oligonucleotide are internucleoside linking groups of Formula XVII.

Embodiment 957. The modified oligonucleotide of embodiment 954, wherein at least 4 of the ten 5′-most linking groups of the modified oligonucleotide are internucleoside linking groups of Formula XVII.

Embodiment 958. The modified oligonucleotide of embodiment 954, wherein at least 5 of the ten 5′-most linking groups of the modified oligonucleotide are internucleoside linking groups of Formula XVII.

Embodiment 959. The modified oligonucleotide of embodiment 954, wherein at least 6 of the ten 5′-most linking groups of the modified oligonucleotide are internucleoside linking groups of Formula XVII.

Embodiment 960. The modified oligonucleotide of embodiment 954, wherein the two 5′-most internucleoside linking groups are internucleoside linking groups of Formula XVII.

Embodiment 961. The modified oligonucleotide of any of embodiments 943-960, wherein at least one of the ten 3′-most internucleoside linking groups of the modified oligonucleotide is an internucleoside linking group of Formula XVII.

Embodiment 962. The modified oligonucleotide of embodiment 961, wherein at least 2 of the ten 3′-most internucleoside linking groups of the modified oligonucleotide are internucleoside linking groups of Formula XVII.

Embodiment 963. The modified oligonucleotide of embodiment 961, wherein at least 3 of the ten 3′-most internucleoside linking groups are internucleoside linking groups of Formula XVII.

Embodiment 964. The modified oligonucleotide of embodiment 961, wherein at least 4 of the ten 3′-most internucleoside linking groups are internucleoside linking groups of Formula XVII.

Embodiment 965. The modified oligonucleotide of embodiment 961, wherein at least 5 of the ten 3′-most internucleoside linking groups are internucleoside linking 961 of Formula XVII.

Embodiment 966. The modified oligonucleotide of embodiment 961, wherein at least 6 of the ten 3′-most internucleoside linking groups are internucleoside linking groups of Formula XVII.

Embodiment 967. The modified oligonucleotide of embodiment 961, wherein the two 3′-most internucleoside linking groups of the modified oligonucleotide are internucleoside linking groups of Formula XVII.

Embodiment 968. The modified oligonucleotide of any of embodiments 943-953, wherein the modified oligonucleotide comprises at least one block of at least 3 consecutive internucleoside linking groups of Formula XVII.

Embodiment 969. The modified oligonucleotide of any of embodiments 943-953, wherein the modified oligonucleotide comprises at least one block of at least 4 consecutive internucleoside linking groups of Formula XVII.

Embodiment 970. The modified oligonucleotide of any of embodiments 943-953, wherein the modified oligonucleotide comprises at least one block of at least 5 consecutive internucleoside linking groups of Formula XVII.

Embodiment 971. The modified oligonucleotide of any of embodiments 943-953, wherein the modified oligonucleotide comprises at least one block of at least 6 consecutive internucleoside linking groups of Formula XVII.

Embodiment 972. The modified oligonucleotide of any of embodiments 968-971, wherein at least one block of consecutive internucleoside linking groups of Formula XVII is at the 5′ end of the modified oligonucleotide.

Embodiment 973. The modified oligonucleotide of any of embodiments 968-971, wherein at least one block of consecutive internucleoside linking groups of Formula XVII is at the 3′ end of the modified oligonucleotide.

Embodiment 974. The modified oligonucleotide of any of embodiments 943-973, wherein for each internucleoside linking group of Formula XVII, R₁ is H and T is SO₂Me.

Embodiment 975. The modified oligonucleotide of any of embodiments 943-953, wherein the internucleoside linkage motif is selected from: aaaaaasssssssss, sssssaaaaaassss, or sssssssssaaaaaa, wherein each “a” represents an internucleoside linkage of Formula XVII, each “s” represents a phosphorothioate internucleoside linkage, and each “o” represents a phosphodiester internucleoside linkage.

Embodiment 976. The modified oligonucleotide of embodiment 975, wherein each “a” represents a mesyl phosphoramidate internucleoside linkage.

Embodiment 977. The modified oligonucleotide of any of embodiments 705-841, wherein the modified oligonucleotide comprises a deoxy region consisting of 6-11 linked nucleosides wherein each nucleoside of the deoxy region is either a modified nucleoside or a stereo-standard DNA nucleoside and wherein at least 3 contiguous nucleosides of the deoxy region are stereo-standard DNA nucleosides and not more than three nucleosides of the deoxy region are modified nucleosides.

Embodiment 978. The modified oligonucleotide of embodiment 977, wherein at least 5 contiguous nucleosides of the deoxy region are stereo-standard DNA nucleosides.

Embodiment 979. The modified oligonucleotide of embodiment 977, wherein at least 6 contiguous nucleosides of the deoxy region are stereo-standard DNA nucleosides.

Embodiment 980. The modified oligonucleotide of embodiment 977, wherein at least 7 contiguous nucleosides of the deoxy region are stereo-standard DNA nucleosides.

Embodiment 981. The modified oligonucleotide of embodiment 977, wherein at least 8 contiguous nucleosides of the deoxy region are stereo-standard DNA nucleosides.

Embodiment 982. The modified oligonucleotide of any of embodiments 977-981, wherein the deoxy region consists of 8-10 linked nucleosides.

Embodiment 983. The modified oligonucleotide of any of embodiments 977-981, wherein the deoxy region consists of 9 linked nucleosides.

Embodiment 984. The modified oligonucleotide of any of embodiments 977-981, wherein the deoxy region consists of 10 linked nucleosides.

Embodiment 985. The modified oligonucleotide of any of embodiments 977-981, wherein the deoxy region consists of 11 linked nucleosides.

Embodiment 986. The modified oligonucleotide of any of embodiments 977-981, wherein at least 6 nucleosides of the deoxy region are stereo-standard DNA nucleosides.

Embodiment 987. The modified oligonucleotide of any of embodiments 977-981, wherein at least 7 nucleosides of the deoxy region are stereo-standard DNA nucleosides.

Embodiment 988. The modified oligonucleotide of any of embodiments 977-981, wherein at least 8 nucleosides of the deoxy region are stereo-standard DNA nucleosides.

Embodiment 989. The modified oligonucleotide of any of embodiments 977-981, wherein at least 9 nucleosides of the deoxy region are stereo-standard DNA nucleosides.

Embodiment 990. The modified oligonucleotide of any of embodiments 977-989 wherein exactly two nucleosides of the deoxy region are modified nucleosides.

Embodiment 991. The modified oligonucleotide of any of embodiments 977-989 wherein exactly one nucleoside of the deoxy region is a modified nucleoside.

Embodiment 992. The modified oligonucleotide of any of embodiments 977-991 wherein at least one modified nucleoside of the deoxy region is a stereo-standard modified nucleoside or bicyclic nucleoside selected from a β-D-LNA nucleoside, an α-L-LNA nucleoside, an ENA nucleoside, a cEt nucleoside, a 2′-MOE nucleoside, a 2′-OMe nucleoside, a 2′-F nucleoside, and a 5′-alkyl nucleoside.

Embodiment 993. The modified oligonucleotide of any of embodiments 977-991, wherein at least one modified nucleoside of the deoxy region is stereo-non-standard nucleoside.

Embodiment 994. The modified oligonucleotide of embodiment 993, wherein the at least one is stereo-non-standard nucleoside of the deoxy region is a stereo-non-standard DNA nucleoside.

Embodiment 995. The modified oligonucleotide of embodiment 994, wherein the stereo-non-standard DNA nucleoside is selected from a stereo-non-standard DNA nucleoside having: Formula I, Formula II, Formula III, Formula IV, Formula V, Formula VI, and Formula VII.

Embodiment 996. The modified oligonucleotide of embodiment 995, wherein the stereo-non-standard DNA nucleoside is selected from a stereo-non-standard DNA nucleoside having: Formula V and Formula II.

Embodiment 997. The modified oligonucleotide of embodiment 996, wherein at least one stereo-non-standard nucleoside of the deoxy region is a substituted stereo-non-standard nucleoside.

Embodiment 998. The modified oligonucleotide of embodiment 997, wherein at least one substituted stereo-non-standard nucleoside has a 2′-substituent selected from: 2′-MOE, 2′-OMe, 2′-F, or 2′-OH.

Embodiment 999. The modified oligonucleotide of any of embodiments 977-998, wherein the 2^{nd} nucleoside from the 5′-end of the deoxy region is a modified nucleoside.

Embodiment 1000. The modified oligonucleotide of any of embodiments 977-998, wherein the 3^{rd} nucleoside from the 5′-end of the deoxy region is a modified nucleoside.

Embodiment 1001. The modified oligonucleotide of any of embodiments 977-998 wherein the 4^{th} nucleoside from the 5′-end of the deoxy region is a modified nucleoside.

Embodiment 1002. The modified oligonucleotide of any of embodiments 999-1001, wherein the modified nucleoside in the deoxy region is a 2′-OMe nucleoside.

Embodiment 1003. The modified oligonucleotide of any of embodiments 977-989, wherein each nucleoside of the deoxy region is a stereo-standard DNA nucleoside.

Embodiment 1004. The modified oligonucleotide of any of embodiments 977-1003 wherein at least one internucleoside linking group within the deoxy region is an internucleoside linking group of Formula XVII.

Embodiment 1005. The modified oligonucleotide of any of embodiments 977-1004, wherein the internucleoside linking group linking the 1^{st} and 2^{nd} nucleosides of the deoxy region as counted from the 5′-end of the deoxy region is an internucleoside linking group of Formula XVII.

Embodiment 1006. The modified oligonucleotide of any of embodiments 977-1005, wherein the internucleoside linking group linking the 2^{nd} and 3^{rd} nucleosides of the deoxy region as counted from the 5′-end of the deoxy region is an internucleoside linking group of Formula XVII.

Embodiment 1007. The modified oligonucleotide of any of embodiments 977-1006, wherein the internucleoside linking group linking the 3^{rd} and 4^{th} nucleosides of the deoxy region as counted from the 5′-end of the deoxy region is an internucleoside linking group of Formula XVII.

Embodiment 1008. The modified oligonucleotide of any of embodiments 977-1007, wherein the internucleoside linking group linking the 4^{th} and 5^{th} nucleosides of the deoxy region as counted from the 5′-end of the deoxy region is an internucleoside linking group of Formula XVII.

Embodiment 1009. The modified oligonucleotide of any of embodiments 977-1008, wherein one internucleoside linking group in the deoxy region is a linking group of Formula XVII and the other internucleoside linking groups of the deoxy region are independently selected from phosphodiester and phosphorothioate internucleoside linking groups.

Embodiment 1010. The modified oligonucleotide of any of embodiments 977-1008, wherein two internucleoside linking groups in the deoxy region are linking groups of Formula XVII and the other internucleoside linking groups of the deoxy region are independently selected from phosphodiester and phosphorothioate internucleoside linking groups.

Embodiment 1011. The modified oligonucleotide of any of embodiments 977-1008, wherein three internucleoside linking groups in the deoxy region are linking groups linking groups of Formula XVII and the other internucleoside linking groups of the deoxy region are independently selected from phosphodiester and phosphorothioate internucleoside linking groups.

Embodiment 1012. The modified oligonucleotide of any of embodiments 977-1008, wherein four internucleoside linking groups in the deoxy region are linking groups linking groups of Formula XVII and the other internucleoside linking groups of the deoxy region are each phosphodiester or phosphorothioate internucleoside linking groups.

Embodiment 1013. The modified oligonucleotide of any of embodiments 1009-1012, wherein the internucleoside linking groups of Formula XVII are linking the 1^{st} and 2^{nd}, 2^{nd} and 3^{rd}, 3^{rd} and 4^{th}, and/or the 4^{th} and 5^{th} nucleosides of the deoxy region, as counted from the 5′-end of the deoxy region.

Embodiment 1014. The modified oligonucleotide of any of embodiments 877-1013, wherein the deoxy region comprises at least one region having structure A, B, C, D, or E.

Embodiment 1015. The modified oligonucleotide of embodiment 1014, wherein the region having structure A, B, C, D, or E is at the 3′ end of the deoxy region.

Embodiment 1016. The modified oligonucleotide of embodiment 1014, wherein the region having structure A, B, C, D, or E is at the 5′ end of the deoxy region.

Embodiment 1017. The modified oligonucleotide of any of embodiments 877-1016, wherein the deoxy region comprises at least one region having the formula (N_{g1})_{L1}(N_{g2})_{L2}(N_{g3})_{L3}, wherein each N_{g} is a nucleoside and each L is an internucleoside linking group; wherein each of L₁, and L₂ is a phosphodiester internucleoside linking group, a phosphorothioate intemucleoside linking group, or an internucleoside linking group of Formula XVII:

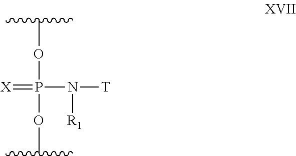

wherein L₃ is absent or is a phosphodiester internucleoside linking group, a phosphorothioate internucleoside linking group, or an internucleoside linking group of Formula XVII;
wherein at least one of L₁, L₂, and L₃ an internucleoside linking group of Formula XVII; and at least one of L₁, L₂, and L₃ is a phosphorothioate or a phosphodiester internucleoside linking group,
   wherein independently for each internucleoside linking group of the modified oligonucleotide having Formula XVII:
   X is selected from O or S;
   R₁ is selected from H, C₁-C₆ alkyl, and substituted C₁-C₆ alkyl; and
   T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
   R₃ is selected from an aryl, a substituted aryl, CH₃, N(CH₃)₂, OCH₃ and a conjugate;
   R₄ is selected from OCH₃, OH, C₁-C₆ alkyl, substituted C₁-C₆ alkyl and a conjugate; and
   R₅ is selected from OCH₃, OH, C₁-C₆ alkyl, and substituted C₁-C₆ alkyl.

Embodiment 1018. The modified oligonucleotide of embodiment 1017, wherein the region having the formula (N_{g1})_{L1}(N_{g2})_{L2}(N_{g3})_{L3} is at the 3′ end of the deoxy region.

Embodiment 1019. The modified oligonucleotide of embodiment 1017, wherein the region having the formula (N_{g1})_{L1}(N_{g2})_{L2}(N_{g3})_{L3} is at the 5′ end of the deoxy region.

Embodiment 1020. The modified oligonucleotide of any of embodiments 1004-1019, wherein for each internucleoside linkage of Formula XVII, R₁ is H and T is SO₂Me.

Embodiment 1021. The modified oligonucleotide of any of embodiments 877-1020 wherein the deoxy region is flanked on the 5′ side by a 5′-region consisting of 1-6 linked 5′-region nucleosides and on the 3′ side by a 3′-region consisting of 1-6 linked 3′-region nucleosides; wherein
the 3′-most nucleoside of the 5′-region comprises a modified sugar moiety; and
the 5′-most nucleoside of the 3′-region comprises a modified sugar moiety.

Embodiment 1022. The modified oligonucleotide of embodiment 1021, wherein the deoxy region consists of 7-11 linked nucleosides, and has the formula:
(N_{d1})_{L1}(N_{d2})_{L2}(N_{d3})_{L3}(N_{d4})_{L4}[(N_{d})_{L5}]_{q};
wherein N_{d1}, N_{d2}, N_{d3}, N_{d4} are independently selected from among
a stereo-standard DNA nucleoside, a stereo-non-standard DNA nucleoside, or a 2′-substituted nucleoside;
with the proviso that no more than one of N_{d1}, N_{d2}, N_{d3}, or N_{d4} is a 2′-substituted nucleoside;
each N_{d} is independently selected from among a stereo-standard DNA nucleoside and a stereo-non-standard DNA nucleoside;
q is from 3-8;
wherein each of L₁, L₂, L₃, L₄, and each L₅ is an internucleoside linkage;
wherein at least two of L₁, L₂, L₃, L₄ are internucleoside linkages of Formula XVII.

Embodiment 1023. The modified oligonucleotide of embodiment 1022, wherein one of N_{d1}, N_{d}2, N_{d3}, or N_{d4} is a 2′-substituted nucleoside.

Embodiment 1024. The modified oligonucleotide of embodiment 1023, wherein the 2′-substituted nucleoside is a 2′-OMe nucleoside.

Embodiment 1025. The modified oligonucleotide of embodiment 1024, wherein the 2′-OMe nucleoside is a stereo-standard 2′-OMe nucleoside.

Embodiment 1026. The modified oligonucleotide of any of embodiments 1022-1025, wherein the 2′-substituted nucleoside is N_{d2}.

Embodiment 1027. The modified oligonucleotide of embodiment 1022, wherein each of N_{d1}, N_{d2}, N_{d3}, N_{d4} and each N_{d} is a DNA nucleoside.

Embodiment 1028. The modified oligonucleotide of embodiment 1027, wherein each DNA nucleoside is a stereo-standard DNA nucleoside.

Embodiment 1029. The modified oligonucleotide of any of embodiments 1022-1028, wherein L₁ and L₂ are internucleoside linkages of Formula XVII.

Embodiment 1030. The modified oligonucleotide of any of embodiments 1022-1028, wherein L₂ and L₃ are internucleoside linkages of Formula XVII.

Embodiment 1031. The modified oligonucleotide of any of embodiments 1022-1028, wherein L₃ and L₄ are internucleoside linkages of Formula XVII.

Embodiment 1032. The modified oligonucleotide of any of embodiments 1022-1028, wherein L₁, L₂, and L₃ are internucleoside linkages of Formula XVII.

Embodiment 1033. The modified oligonucleotide of any of embodiments 1022-1028, wherein L₂, L₃, and L₄ are internucleoside linkages of Formula XVII.

Embodiment 1034. The modified oligonucleotide of any of embodiments 1022-1028, wherein L₁, L₂, L₃, and L₄ are internucleoside linkages of Formula XVII.

Embodiment 1035. The modified oligonucleotide of embodiments 1029-1034, wherein each internucleoside linkage that is not an internucleoside linkage of Formula XVII is a phosphorothioate internucleoside linkage.

Embodiment 1036. The modified oligonucleotide of any of embodiments 1029-1035, wherein for each internucleoside linkage of Formula XVII, R₁ is H and T is SO₂Me Embodiment 1037. The modified oligonucleotide of any of embodiments 1021-1036, wherein the 5′-region consists of 2-5 linked nucleosides.

Embodiment 1038. The modified oligonucleotide of embodiment 1037, wherein the 5′-region consists of 3 linked nucleosides.

Embodiment 1039. The modified oligonucleotide of embodiment 1037, wherein the 5′-region consists of 5 linked nucleosides.

Embodiment 1040. The modified oligonucleotide of any of embodiments 1021-1039 wherein each nucleoside of the 5′-region is a modified nucleoside.

Embodiment 1041. The modified oligonucleotide of any of embodiments 1021-1040, wherein each nucleoside of the 5′-region is a modified nucleoside comprising a modified sugar.

Embodiment 1042. The modified oligonucleotide of any of embodiments 1021-1041, wherein at least one nucleoside of the 5′-region comprises a 2′-substituted furanosyl sugar moiety.

Embodiment 1043. The modified oligonucleotide of any of embodiments 1021-1041, wherein each nucleoside of the 5′-region comprises a 2′-substituted furanosyl sugar moiety.

Embodiment 1044. The modified oligonucleotide of any of embodiments 1021-1043, wherein each 2′-substituted furanosyl sugar moiety of the 5′-region has a 2′-substituent selected from among 2′-MOE, 2′-OMe, and 2′-NMA.

Embodiment 1045. The modified oligonucleotide of any of embodiments 1021-1042 or 1044, wherein at least one nucleoside of the 5′-region comprises a bicyclic furanosyl sugar moiety.

Embodiment 1046. The modified oligonucleotide of any of embodiments 1021-1042 or 1044-1045, wherein each nucleoside of the 5′-region comprises a bicyclic furanosyl sugar moiety.

Embodiment 1047. The modified oligonucleotide of embodiment 341 or 342, wherein each bicyclic sugar moiety of the 5′-region is selected from among cEt, LNA, and ENA.

Embodiment 1048. The modified oligonucleotide of embodiment 1047, wherein each bicyclic sugar moiety of the 5′-region is a cEt sugar moiety.

Embodiment 1049. The modified oligonucleotide of any of embodiments 1021-1039, 1042 or 1045, wherein at least one nucleoside of the 5′ region is a stereo-standard DNA nucleoside.

Embodiment 1050. The modified oligonucleotide of any of embodiments 1021-1048, wherein at least one nucleoside of the 5′ region is a stereo-non-standard nucleoside.

Embodiment 1051. The modified oligonucleotide of any of embodiments 1021-1050, wherein each nucleobase of the 5′-region is independently selected from among thymine, uracil, guanine, cytosine, 5-methylcytosine, and adenine.

Embodiment 1052. The modified oligonucleotide of any of embodiments 1021-1051, wherein the 3′-region consists of 2-5 linked nucleosides.

Embodiment 1053. The modified oligonucleotide of embodiment 1052, wherein the 3′-region consists of 3 linked nucleosides.

Embodiment 1054. The modified oligonucleotide of embodiment 1052, wherein the 3′-region consists of 5 linked nucleosides.

Embodiment 1055. The modified oligonucleotide of any of embodiments 1021-1054, wherein each nucleoside of the 3′-region is a modified nucleoside.

Embodiment 1056. The modified oligonucleotide of any of embodiments 1021-1055, wherein each nucleoside of the 3′-region is a modified nucleoside comprising a modified sugar.

Embodiment 1057. The modified oligonucleotide of any of embodiments 1021-1056, wherein at least one nucleoside of the 3′-region comprises a 2′-substituted furanosyl sugar moiety.

Embodiment 1058. The modified oligonucleotide of any of embodiments 1021-1057, wherein each nucleoside of the 3′-region comprises a 2′-substituted furanosyl sugar moiety.

Embodiment 1059. The modified oligonucleotide of any of embodiments 1021-1058, wherein each 2′-substituted furanosyl sugar moiety of the 3′-region has a 2′-substituent selected from among 2′-MOE, 2′-OMe, and 2′-NMA.

Embodiment 1060. The modified oligonucleotide of any of embodiments 1021-1057 or 1059, wherein at least one nucleoside of the 3′-region comprises a bicyclic furanosyl sugar moiety.

Embodiment 1061. The modified oligonucleotide of any of embodiments 1021-1057 or 1059-1060, wherein each nucleoside of the 3′-region comprises a bicyclic furanosyl sugar moiety.

Embodiment 1062. The modified oligonucleotide of embodiment 1060 or 1061, wherein each bicyclic sugar moiety of the 3′-region is selected from among cEt, LNA, and ENA.

Embodiment 1063. The modified oligonucleotide of embodiment 1062, wherein each bicyclic sugar moiety of the 3′-region is a cEt sugar moiety.

Embodiment 1064. The modified oligonucleotide of any of embodiments 1021-1054, 1057 or 1060, wherein at least one nucleoside of the 3′ region is a stereo-standard DNA nucleoside.

Embodiment 1065. The modified oligonucleotide of any of embodiments 1021-1064, wherein at least one nucleoside of the 3′ region is a stereo-non-standard nucleoside.

Embodiment 1066. The modified oligonucleotide of any of embodiments 1021-1065, wherein each nucleobase of the 3′-region is independently selected from among thymine, uracil, guanine, cytosine, 5-methylcytosine, and adenine.

Embodiment 1067. The modified oligonucleotide of any of embodiments 1021-1066 wherein the modified oligonucleotide is a gapmer.

Embodiment 1068. The modified oligonucleotide of any of embodiments 1021-1066, wherein the modified oligonucleotide has a sugar motif selected from kkkddddddddddkkk and kkkdyddddddddkkk, wherein each “k” represents a cEt sugar moiety, “y” represents a 2′-OMe sugar moiety, and each “d” represents a β-D-2′-deoxyribosyl sugar moiety.

Embodiment 1069. The modified oligonucleotide of any of embodiments 1021-1066, wherein the modified oligonucleotide has an internucleoside linkage motif selected from: ssssssssssssssa, sssssssssssssas, ssssssssssssass, sssssssssssasss, ssssssssssassss, sssssssssasssss, ssssssssassssss, sssssssasssssss, ssssssassssssss, sssssasssssssss, ssssassssssssss, sssasssssssssss, ssassssssssssss, sasssssssssssss, assssssssssssss, sssssssssssssaa, ssssssssssssaas, sssssssssssaass, ssssssssssaasss, sssssssssaassss, ssssssssaasssss, sssssssaassssss, ssssssaasssssss, sssssaassssssss, ssssaasssssssss, sssaassssssssss, ssaasssssssssss, saassssssssssss, aasssssssssssss, aaaaaaaaaaaaaaa, ssaaaaaaaaaaass, ssaaaaaaaaaasss, sssaaaaaaaaasss, aassssssssssaaa, sssaaasssssssss, ssssaaassssssss, sssaaaassssssss, ssaaassssssssss, ssaaaasssssssss, ssaaaaassssssss, ssaaaaaasssssss, ssaaaaaaassssss, ssaaaaaaaasssss, ssaaaaaaaaassss, ssssssssssaaass, sssssssssaaaass, ssssssssaaaaass, sssssssaaaaaass, ssssssaaaaaaass, sssssaaaaaaaass, ssssaaaaaaaaass, sssaaaaaaaaaass, ssasasasasasass, sssasasasasasss, ooossssssssssoo, soossssssssssos, aoosssssssssooa, aoasssssssssaoa, aoaaaassssssaoa, aoossssssssssoa, ooasssssssssaoo, aoosaasssssssoa, aossaasssssssoa, aooaaaasssssaoa, aoossssssaaaaoa, sssssaaasssssss, ssssssaaassssss, sssssssaaasssss, ssssssssaaassss, sssssssssaaasss, ssssaaaasssssss, sssssaaaassssss, ssssssaaaasssss, sssssssaaaassss, ssssssssaaaasss, sssaassssssaass, sssaasssssaasss, sssaassssaassss, sssaasssaasssss, sssaassaassssss, sssaasaasssssss, ssaasssssssaass, ssssaasssssaass, sssssaassssaass, ssssssaasssaass, sssssssaassaass, ssssssssaasaass, ssssaasssaassss, sssssaasaasssss, sssssssssssssss, aaassssssssssss, aaassssssssssaa, aaaaaasssssssss, aoooosaasssssssooaa, aoooossssssssssooaa, sooooaassssssssooss, soooosaasssssssooss, soooossaassssssooss, soooossssssaassooss, sssaaaaasssssss, sssssaaaaaassss, or sssssssssaaaaaa wherein each “a” represents an internucleoside linkage of Formula XVII, each “s” represents a phosphorothioate internucleoside linkage, and each “o” represents a phosphodiester internucleoside linkage.

Embodiment 1070. The modified oligonucleotide of embodiment 1069, wherein the modified oligonucleotide has an internucleoside linkage motif selected from: sssaaaassssssss, sssaaasssssssss, ssssaaassssssss, ssssaasssssaass, sssaassssssssss, ssssaasssssssss, sssssaassssssss, or sssssssssaassss, wherein each “a” represents an internucleoside linkage of Formula XVII, each “s” represents a phosphorothioate internucleoside linkage, and each “o” represents a phosphodiester internucleoside linkage.

Embodiment 1071. The modified oligonucleotide of embodiment 1069-1070, wherein each “a” represents a mesyl phosphoramidate internucleoside linkage.

Embodiment 1072. The modified oligonucleotide of any of embodiments 705-841, wherein the modified oligonucleotide is a CRISPR compound.

Embodiment 1073. The modified oligonucleotide of embodiment 1072, wherein the CRISPR compound consists of 20-50 or 29-32 linked nucleosides.

Embodiment 1074. The modified oligonucleotide of any of embodiments 794-1073, wherein each X is O.

Embodiment 1075. The modified oligonucleotide of any of embodiments 794-1073, wherein each X is S.

Embodiment 1076. The modified oligonucleotide of any of embodiments 794-1075, wherein at least one R₁ is H.

Embodiment 1077. The modified oligonucleotide of any of embodiments 794-1075, wherein at least one R₁ is a C₁-C₆ alkyl.

Embodiment 1078. The modified oligonucleotide of embodiment 1077, wherein the at least one R₁ is methyl.

Embodiment 1079. The modified oligonucleotide of any of embodiments 794-1075, at least one R₁ is a substituted C₁-C₆ alkyl.

Embodiment 1080. The modified oligonucleotide of any of embodiments 794-1079, wherein at least one T comprises a conjugate group.

Embodiment 1081. The modified oligonucleotide of embodiment 1080, wherein the conjugate group comprises a cell-targeting moiety.

Embodiment 1082. The modified oligonucleotide of embodiment 1080, wherein the conjugate group comprises a carbohydrate or carbohydrate cluster.

Embodiment 1083. The modified oligonucleotide of any of embodiments 1080-1082, wherein the conjugate group comprises at least one GalNAc.

Embodiment 1084. The modified oligonucleotide of embodiment 1080, wherein the conjugate group comprises a C₁₀-C₂₀ alkyl chain.

Embodiment 1085. The modified oligonucleotide of embodiment 1084, wherein the conjugate group comprises C₁₆ alkyl.

Embodiment 1086. The modified oligonucleotide of any of embodiments 794-1085, wherein at least one T does not comprise a conjugate group.

Embodiment 1087. The modified oligonucleotide of any of embodiments 794-1079, wherein each T does not comprise a conjugate group.

Embodiment 1088. The modified oligonucleotide of any of embodiments 794-1087, wherein at least one T is SO₂R₂.

Embodiment 1089. The modified oligonucleotide of embodiment 1088, wherein R₂ is an aryl.

Embodiment 1090. The modified oligonucleotide of embodiment 1088, wherein R₂ is a substituted aryl.

Embodiment 1091. The modified oligonucleotide of embodiment 1088, wherein R₂ is a heterocycle.

Embodiment 1092. The modified oligonucleotide of embodiment 1088, wherein R₂ is a substituted heterocycle.

Embodiment 1093. The modified oligonucleotide of embodiment 1088, wherein R₂ is an aromatic heterocycle.

Embodiment 1094. The modified oligonucleotide of embodiment 1088, wherein R₂ is a substituted aromatic heterocycle.

Embodiment 1095. The modified oligonucleotide of embodiment 1088, wherein R₂ is a diazole.

Embodiment 1096. The modified oligonucleotide of embodiment 1088, wherein R₂ is a substituted diazole.

Embodiment 1097. The modified oligonucleotide of embodiment 1088, wherein R₂ is an amine.

Embodiment 1098. The modified oligonucleotide of embodiment 1088, wherein R₂ is a substituted amine.

Embodiment 1099. The modified oligonucleotide of embodiment 1088, wherein R₂ is a C₁-C₆ alkoxy, C₁-C₆ alkenyl, or C₁-C₆ alkynyl.

Embodiment 1100. The modified oligonucleotide of embodiment 1088, wherein R₂ is C₁-C₂₀, C₁-C₆, C₂-C₂₀, C₂-C₆, or C₁₀-C₂₀ alkyl.

Embodiment 1101. The modified oligonucleotide of embodiment 1088, wherein R₂ is substituted C₁-C₂₀, C₁-C₆, C₂-C₂₀, C₂-C₆, or C₁₀-C₂₀ alkyl.

Embodiment 1102. The modified oligonucleotide of embodiment 1088, wherein R₂ comprises a carbohydrate or carbohydrate cluster.

Embodiment 1103. The modified oligonucleotide of embodiment 1088, wherein R₂ comprises at least one GalNAc.

Embodiment 1104. The modified oligonucleotide of embodiment 1088, wherein T is:

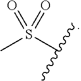 [SMILES: *S(C)(=O)=O]

Embodiment 1105. The modified oligonucleotide of embodiment 1088, wherein T is:

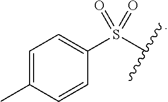 [SMILES: *S(=O)(=O)c1ccc(C)cc1]

Embodiment 1106. The modified oligonucleotide of embodiment 1088, wherein T is:

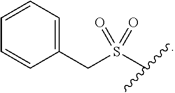 [SMILES: *S(=O)(=O)Cc1ccccc1]

Embodiment 1107. The modified oligonucleotide of embodiment 1088, wherein T is:

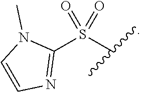 [SMILES: *S(=O)(=O)c1nccn1C]

Embodiment 1108. The modified oligonucleotide of embodiment 1088, wherein T is:

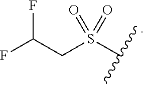 [SMILES: *S(=O)(=O)CC(F)F]

Embodiment 1109. The modified oligonucleotide of embodiment 1088, wherein T is:

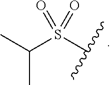 [SMILES: *S(=O)(=O)C(C)C]

Embodiment 1110. The modified oligonucleotide of embodiment 1088, wherein T is:

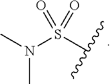 [SMILES: *S(=O)(=O)N(C)C]

Embodiment 1111. The modified oligonucleotide of embodiment 1088, wherein T is:

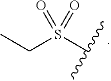 [SMILES: *S(=O)(=O)CC]

Embodiment 1112. The modified oligonucleotide of embodiment 1088, wherein T is:

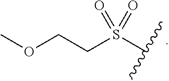 [SMILES: *S(=O)(=O)CCOC]

Embodiment 1113. The modified oligonucleotide of embodiment 1088, wherein T is:

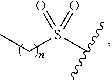

wherein n is from 2 to 20.

Embodiment 1114. The modified oligonucleotide of embodiment 1113, wherein n is 15.

Embodiment 1115. The modified oligonucleotide of any of embodiments 794-1114, wherein at least one T is C(═O)R₃.

Embodiment 1116. The modified oligonucleotide of embodiment 1115, wherein R₃ is an aryl.

Embodiment 1117. The modified oligonucleotide of embodiment 1115, wherein R₃ is a substituted aryl.

Embodiment 1118. The modified oligonucleotide of embodiment 1115, wherein R₃ is CH₃.

Embodiment 1119. The modified oligonucleotide of embodiment 1115, wherein R₃ is N(CH₃)₂.

Embodiment 1120. The modified oligonucleotide of embodiment 1115, wherein R₃ is OCH₃.

Embodiment 1121. The modified oligonucleotide of embodiment 1115, wherein R₃ is a C₁-C₆ alkoxy.

Embodiment 1122. The modified oligonucleotide of embodiment 1115, wherein R₃ is C₁-C₂₀, C₁-C₆, C₂-C₂₀, C₂-C₆, or C₁₀-C₂₀ alkyl.

Embodiment 1123. The modified oligonucleotide of embodiment 1115, wherein R₃ is substituted C₁-C₂₀, C₁-C₆, C₂-C₂₀, C₂-C₆, or C₁₀-C₂₀ alkyl.

Embodiment 1124. The modified oligonucleotide of embodiment 1115, wherein R₃ comprises a carbohydrate or carbohydrate cluster.

Embodiment 1125. The modified oligonucleotide of embodiment 1115, wherein R₂₃ comprises at least one GalNAc.

Embodiment 1126. The modified oligonucleotide of embodiment 1115, wherein T is:

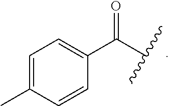 [SMILES: *C(=O)c1ccc(C)cc1]

Embodiment 1127. The modified oligonucleotide of embodiment 1115, wherein T is:

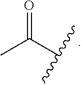 [SMILES: *C(C)=O]

Embodiment 1128. The modified oligonucleotide of embodiment 1115, wherein T is:

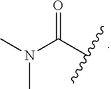 [SMILES: *C(=O)N(C)C]

Embodiment 1129. The modified oligonucleotide of embodiment 1115, wherein T is:

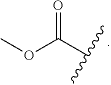 [SMILES: *C(=O)OC]

Embodiment 1130. The modified oligonucleotide of embodiment 1115, wherein T is:

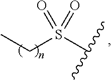

wherein n is from 2 to 20.

Embodiment 1131. The modified oligonucleotide of embodiment 1130, wherein n is 15.

Embodiment 1132. The modified oligonucleotide of any of embodiments 794-1131, wherein at least one T is P(═O)R₄R₅.

Embodiment 1133. The modified oligonucleotide of embodiment 1132, wherein R₄ is OCH₃.

Embodiment 1134. The modified oligonucleotide of embodiment 1132, wherein R₄ is OH.

Embodiment 1135. The modified oligonucleotide of embodiment 1132, wherein R₄ is C₁-C₆ alkyl.

Embodiment 1136. The modified oligonucleotide of embodiment 1132, wherein R₄ is substituted C₁-C₆ alkyl.

Embodiment 1137. The modified oligonucleotide of embodiment 1132, wherein R₄ is C₁-C₂₀, C₁-C₆, C₂-C₂₀, C₂-C₆, or C₁₀-C₂₀ alkyl.

Embodiment 1138. The modified oligonucleotide of embodiment 1132, wherein R₄ is substituted C₁-C₂₀, C₁-C₆, C₂-C₂₀, C₂-C₆, or C₁₀-C₂₀ alkyl.

Embodiment 1139. The modified oligonucleotide of embodiment 1132, wherein R₄ comprises a carbohydrate or carbohydrate cluster.

Embodiment 1140. The modified oligonucleotide of embodiment 1132, wherein R₄ comprises at least one GalNAc.

Embodiment 1141. The modified oligonucleotide of any of embodiments 1132-1140, wherein R₅ is OCH₃.

Embodiment 1142. The modified oligonucleotide of any of embodiments 1132-1140, wherein R₅ is OH.

Embodiment 1143. The modified oligonucleotide of any of embodiments 1132-1140, wherein R₅ is C₁-C₆ alkyl.

Embodiment 1144. The modified oligonucleotide of any of embodiments 1132-1140, wherein R₅ is substituted C₁-C₆ alkyl.

Embodiment 1145. The modified oligonucleotide of embodiment 1132, wherein T is:

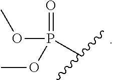 [SMILES: *P(=O)(OC)OC]

Embodiment 1146. The modified oligonucleotide of embodiment 1132, wherein T is:

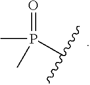 [SMILES: *P(C)(C)=O]

Embodiment 1147. The modified oligonucleotide of embodiment 1132, wherein T is:

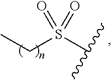

wherein n is from 2 to 20.

Embodiment 1148. The modified oligonucleotide of embodiment 1147, wherein n is 15.

Embodiment 1149. An antisense agent comprising a modified oligonucleotide consisting of 12-50 linked nucleosides linked through internucleoside linking groups, wherein at least one internucleoside linking group is a phosphodiester or a phosphorothioate internucleoside linking group, and wherein at least one of the internucleoside linking groups has Formula XX:

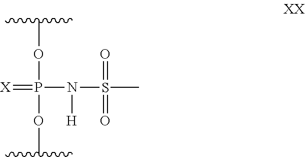

wherein independently for each internucleoside linking group of the modified oligonucleotide having Formula XX, X is selected from O or S.

Embodiment 1150. An antisense agent comprising a modified oligonucleotide, wherein the 5′-terminus of the modified oligonucleotide has Structure F:

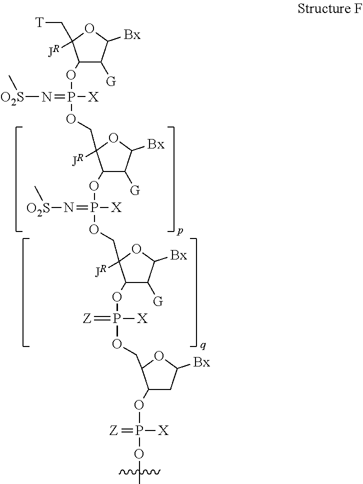

wherein:
p is from 0 to 6;
q is from 0 to 6;
T is OH or a conjugate group;
each Bx is an independently selected heterocyclic base moiety;
each X is independently selected from OH or SH;
each Z is independently selected from O, S, or NSO₂Me;
For each J^{R} and G of the same furanosyl sugar moiety, either J^{R} and G form a J^{R} to G bridge, or J^{R} is H and G is selected from OH, halogen or O—[C(R₆)(R₇)]ₙ[(C═O)ₘ—X^{G}]—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and
C(=Q₂)N(J₁)(J₂);
   Q₂ is O, S or NJ₃;
   each J₁, J₂ and J₃ is, independently, H or C₁-C₆ alkyl.

Embodiment 1151. An antisense agent comprising a modified oligonucleotide, wherein the 3′-terminus of the modified oligonucleotide has Structure G:

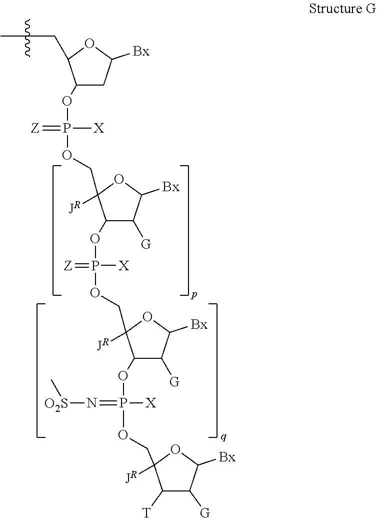

wherein:
p is from 0 to 6;
q is from 1 to 6;
T is OH or a conjugate group;
each Bx is an independently selected heterocyclic base moiety;
each X is independently selected from OH or SH;
each Z is independently selected from O, S, or NSO₂Me;
for each J^{R} and G of the same furanosyl sugar moiety, either J^{R} and G form a J^{R} to G bridge, or J^{R} is H and G is selected from OH, halogen or O—[C(R₆)(R₇)]ₙ[(C═O)ₘ—X^{G}]—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and
C(=Q₂)N(J₁)(J₂);
Q₂ is O, S or NJ₃;
each J₁, J₂ and J₃ is, independently, H or C₁-C₆ alkyl.

Embodiment 1152. The antisense agent of embodiment 1150 or 1151, wherein the sum of p+q is selected from 2, 3, 4, or 5.

Embodiment 1153. An antisense agent comprising a modified oligonucleotide, wherein the 5′-terminus of the modified oligonucleotide has Structure H:

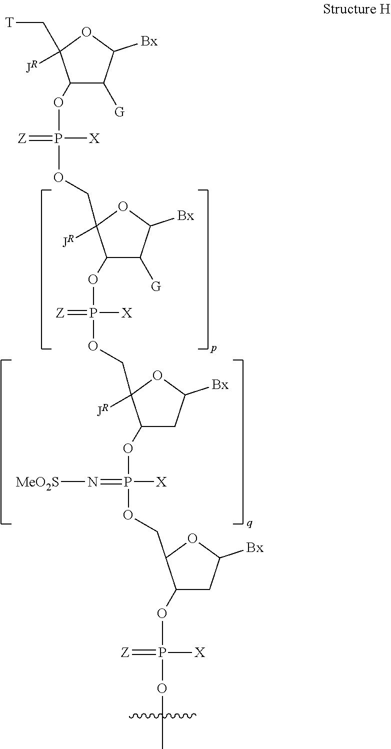

wherein:
p is from 0 to 5;
q is from 1 to 4;
T is OH or a conjugate group;
each Bx is an independently selected heterocyclic base moiety;
each X is independently selected from OH or SH;
each Z is independently selected from O, S, or NSO₂Me;
for each J^{R} and G of the same furanosyl sugar moiety, either J^{R} and G form a J^{R} to G bridge, or J^{R} is H and G is selected from OH, halogen or O—[C(R₆)(R₇)]ₙ[(C═O)ₘ—X^{G}]—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and
C(=Q₂)N(J₁)(J₂);
Q₂ is O, S or NJ₃;
each J₁, J₂ and J₃ is, independently, H or C₁-C₆ alkyl.

Embodiment 1154. An antisense agent comprising a modified oligonucleotide, wherein the 5′-terminus of the modified oligonucleotide has Structure I:

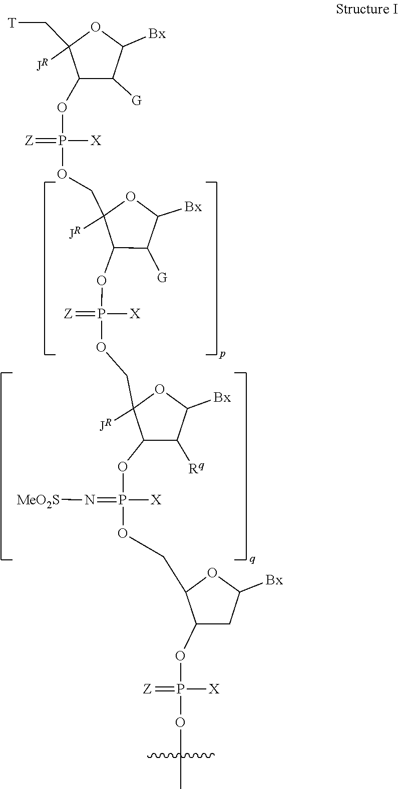

wherein:
p is from 0 to 5;
q is from 1 to 4;
T is OH or a conjugate group;
each Bx is an independently selected heterocyclic base moiety;
each X is independently selected from OH or SH;
each Z is independently selected from O, S, or NSO₂Me;
each R^{q} is H or exactly one R^{q} is OMe and the other R^{q} are H;
for each J^{R} and G of the same furanosyl sugar moiety, either J^{R} and G form a J^{R} to G bridge, or J^{R} is H and G is selected from OH, halogen or O—[C(R₆)(R₇)]ₙ[(C═O)ₘ—X^{G}]—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and
C(=Q₂)N(J₁)(J₂);
Q₂ is O, S or NJ₃;
each J₁, J₂ and J₃ is, independently, H or C₁-C₆ alkyl.

Embodiment 1155. The antisense agent of embodiment 1154, wherein exactly one R^{q} is —OMe.

Embodiment 1156. The antisense agent of any of embodiments 1153-1155, wherein the sum of p+q is 2, 3, or 4.

Embodiment 1157. An antisense agent comprising a modified oligonucleotide, wherein the 3′-terminus of the modified oligonucleotide has Structure J:

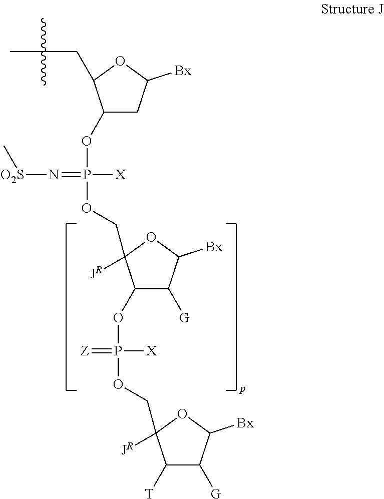

wherein:
p is from 0 to 6;
T is OH or a conjugate group;
each Bx is an independently selected heterocyclic base moiety;
each X is independently selected from OH or SH;
each Z is independently selected from O, S, or NSO₂Me;
For each J^{R} and G of the same furanosyl sugar moiety, either J^{R} and G form a J^{R} to G bridge, or J^{R} is H and G is selected from OH, halogen or O—[C(R₆)(R₇)]—[(C═O)ₘ—X^{G}]ⱼ—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and
C(=Q₂)N(J₁)(J₂);
Q₂ is O, S or NJ₃;
each J₁, J₂ and J₃ is, independently, H or C₁-C₆ alkyl.

Embodiment 1158. The antisense agent of embodiment 1157, wherein p is 2, 3, or 4.

Embodiment 1159. The antisense agent of any of embodiments 1150-1158, wherein each J^{R} is H and each G is OCH₂CH₂OCH₃.

Embodiment 1160. The antisense agent of any of embodiments 1150-1158, wherein each J^{R} is H and each G is OCH₃.

Embodiment 1161. The antisense agent of any of embodiments 1150-1158, wherein each J^{R} and G form a J^{R} to G bridge.

Embodiment 1162. The antisense agent of embodiment 1161, wherein the J^{R} to G bridge has the formula —CH(CH₃)—O—.

Embodiment 1163. The antisense agent of embodiment 1149, wherein the antisense agent is an RNAi agent.

Embodiment 1164. The RNAi agent of embodiment 1163, wherein the RNAi agent is a single-stranded RNAi agent comprising an RNAi antisense modified oligonucleotide, wherein the RNAi antisense modified oligonucleotide is a modified oligonucleotide of embodiment 1149.

Embodiment 1165. The RNAi agent of embodiment 1163, wherein the RNAi agent is an oligonucleotide duplex comprising an RNAi antisense modified oligonucleotide and an RNAi sense modified oligonucleotide, wherein the RNAi antisense modified oligonucleotide and/or the RNAi sense modified oligonucleotide is a modified oligonucleotide of embodiment 1149.

Embodiment 1166. The RNAi agent of any of embodiments 1164-1165, wherein the 5′-terminus of the RNAi antisense oligonucleotide has structure K:

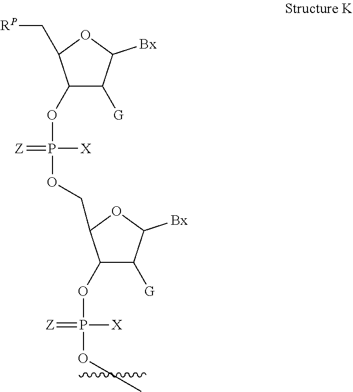

wherein:
R^{P} is a phosphate or stabilized phosphate group;
each Bx is an independently selected heterocyclic base moiety;
each X is independently selected from OH or SH;
each Z is selected from O, S, or NSO₂Me;
at least one Z is NSO₂Me;
each G is independently selected from OH, halogen or O—[C(R₆)(R₇)]ₙ—[(C═O)ₘ—X^{G}]ⱼ—R₈;
each R₆ and R₇ is, independently, H, halogen, C₁-C₆ alkyl or substituted C₁-C₆ alkyl;
each X^{G} is O, S or N(E₁); R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and
C(=Q₂)N(J₁)(J₂);
Q₂ is O, S or NJ₃;
each J₁, J₂ and J₃ is, independently, H or C₁-C₆ alkyl.

Embodiment 1167. The RNAi agent of embodiment 1166, wherein the stabilized phosphate group is 5′-vinyl phosphonate or 5′-cyclopropyl phosphonate.

Embodiment 1168. The RNAi agent of embodiment 1166 or 1167, wherein each G within structure K is independently selected from F or OMe.

Embodiment 1169. The RNAi agent of any of embodiments 1164-1168, wherein the 3′-terminus of the RNAi antisense oligonucleotide has structure L:

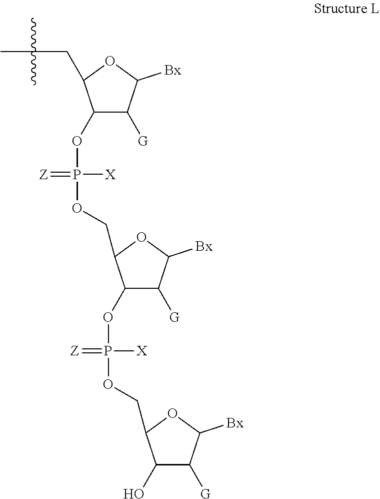

wherein:
each Bx is an independently selected heterocyclic base moiety;
each X is independently selected from OH or SH;
each Z is selected from O, S, or NSO₂Me;
at least one Z is NSO₂Me;
each G is independently selected from OH, halogen or O—[C(R₆)(R₇)]ₙ[(C═O)ₘ—X^{G}]ⱼ—R₈;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and
C(=Q₂)N(J₁)(J₂);
Q₂ is O, S or NJ₃;
each J₁, J₂ and J₃ is, independently, H or C₁-C₆ alkyl.

Embodiment 1170. The RNAi agent of embodiment 1169, wherein each G within Structure L of the RNAi antisense oligonucleotide is independently selected from F or OMe.

Embodiment 1171. The RNAi agent of any of embodiments 1164-1170, wherein at least one region of the RNAi antisense oligonucleotide has structure M:

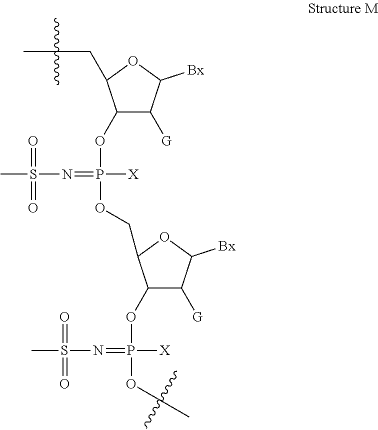

wherein:
each Bx is an independently selected heterocyclic base moiety;
each X is independently selected from OH or SH;
each G is independently selected from OH, halogen or O—[C(R₆)(R₇)]ₙ—[(C═O)ₘ—X^{G}],R₈;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and
C(=Q₂)N(J₁)(J₂);
Q₂ is O, S or NJ₃;
each J₁, J₂ and J₃ is, independently, H or C₁-C₆ alkyl.

Embodiment 1172. The RNAi agent of embodiment 1171, wherein each G of Structure M within the RNAi antisense oligonucleotide is selected from F or OMe.

Embodiment 1173. The RNAi agent of embodiment 1172, wherein one G is F and the other G is OMe.

Embodiment 1174. The RNAi agent of any of embodiments 1164-1165 or 1169-1173, wherein the 5′-terminus of the RNAi antisense oligonucleotide has structure N:

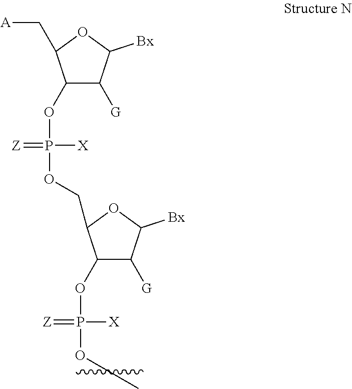

wherein:

A is selected from

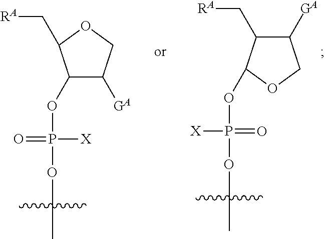

R^{A} is OH, OP(═O)OH, OP(═O)SH, or a stabilized phosphate group;
G^{A} is H, OH, OMe, MOE, or a halogen;
each Bx is an independently selected heterocyclic base moiety;
each X is independently selected from OH or SH;
each G is independently selected from OH, halogen or O—[C(R₆)(R₇)]ₙ[(C═O)ₘ—X^{G}]ⱼ—R₈;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and
C(=Q₂)N(J₁)(J₂);
Q₂ is O, S or NJ₃;
each J₁, J₂ and J₃ is, independently, H or C₁-C₆ alkyl.

Embodiment 1175. The RNAi agent of embodiment 1174, wherein each G within structure N of the RNAi antisense oligonucleotide is selected from F or OMe.

Embodiment 1176. The RNAi agent of any of embodiments 1164-1168 or 1171-1175, wherein the 3′-terminus of the RNAi antisense oligonucleotide has structure O:

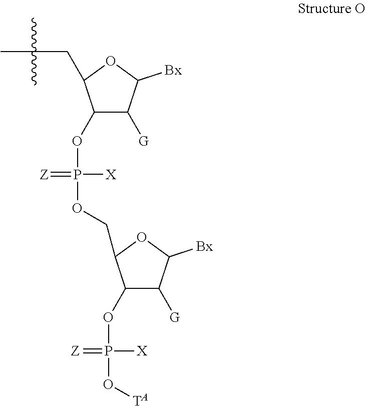

wherein:
T^{A} is selected from

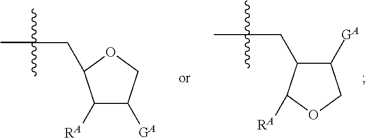

R^{A} G^{A} or R^{A} OP.
R^{A} is OH, OP(═O)OH, OP(═O)SH, or a stabilized phosphate group;
G^{A} is H, OH, OMe, MOE, or a halogen;
each Bx is an independently selected heterocyclic base moiety;
each X is independently selected from OH or SH;
each Z is selected from O, S, or NSO₂Me;
at least one Z is NSO₂Me;
each G is independently selected from OH, halogen or O—[C(R₆)(R₇)]ₙ—[(C═O)ₘ—X^{G}]ⱼ—R₈;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and
C(=Q₂)N(J₁)(J₂);
Q₂ is O, S or NJ₃;
each J₁, J₂ and J₃ is, independently, H or C₁-C₆ alkyl.

Embodiment 1177. The RNAi agent of embodiment 1176, wherein each G within structure O of the RNAi antisense oligonucleotide is selected from F or OMe.

Embodiment 1178. The RNAi agent of embodiment 1165, wherein the 5′-terminus of the RNAi sense oligonucleotide has structure K:

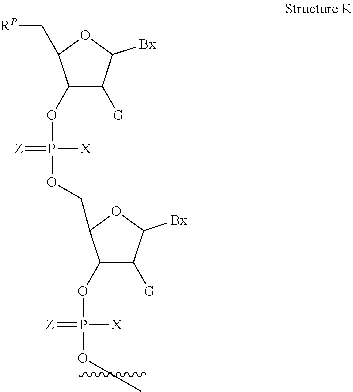

wherein:
R^{P} is a phosphate or stabilized phosphate group;
each Bx is an independently selected heterocyclic base moiety;
each X is independently selected from OH or SH;
each Z is selected from O, S, or NSO₂Me;
at least one Z is NSO₂Me;
each G is independently selected from OH, halogen or O—[C(R₆)(R₇)]ₙ—[(C═O)ₘ—X^{G}]ⱼ—R₈;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and
C(=Q₂)N(J₁)(J₂);
Q₂ is O, S or NJ₃;
each J₁, J₂ and J₃ is, independently, H or C₁-C₆ alkyl.

Embodiment 1179. The RNAi agent of embodiment 1178, wherein the stabilized phosphate group is 5′-vinyl phosphonate or 5′-cyclopropyl phosphonate.

Embodiment 1180. The RNAi agent of embodiment 1178 or 1179, wherein each G within structure K is independently selected from F or OMe.

Embodiment 1181. The RNAi agent of any of embodiments 1165 or 1178-1180, wherein the 3′-terminus of the RNAi sense oligonucleotide has structure L:

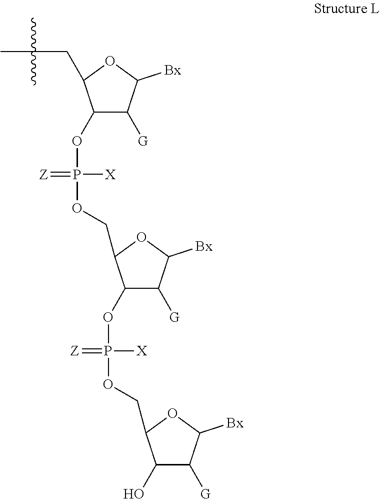

wherein:
   each Bx is an independently selected heterocyclic base moiety;
   each X is independently selected from OH or SH;
   each Z is selected from O, S, or NSO₂Me;
at least one Z is NSO₂Me;
each G is independently selected from OH, halogen or O—[C(R₆)(R₇)]ₙ[(C═O)ₘ—X^{G}]ⱼ—R₈;
   each R₆ and R₇ is, independently, H, halogen, C₁-C₆ alkyl or substituted C₁-C₆ alkyl;
   each X^{G} is O, S or N(E₁);
   R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
   E₁, E₂ and E₃ are each, independently, H, C₁-C₆ alkyl or substituted C₁-C₆ alkyl;
   n is from 1 to 6;
   m is 0 or 1;
   j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
   Q₂ is O, S or NJ₃;
   each J₁, J₂ and J₃ is, independently, H or C₁-C₆ alkyl.

Embodiment 1182. The RNAi agent of embodiment 1181, wherein each G within Structure L of the RNAi sense oligonucleotide is independently selected from F or OMe.

Embodiment 1183. The RNAi agent of any of embodiments 1165 or 1178-1182 wherein at least one region of the RNAi sense oligonucleotide has structure M:

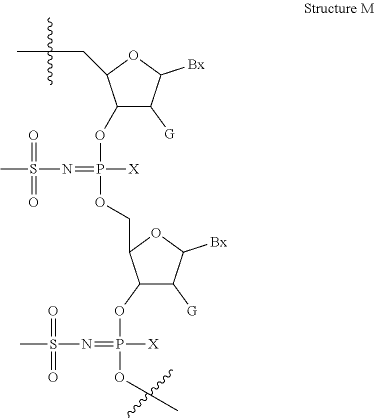

wherein:
each Bx is an independently selected heterocyclic base moiety;
each X is independently selected from OH or SH;
each G is independently selected from OH, halogen or O—[C(R₆)(R₇)]ₙ[(C═O)ₘ—X^{G}]ⱼ—R₈; each R₆ and R₇ is, independently, H, halogen, C₁-C₆ alkyl or substituted C₁-C₆ alkyl;
each X^{G} is O, S or N(E₁);
   R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

Embodiment 1184. The RNAi agent of embodiment 1183, wherein each G of Structure M within the RNAi sense oligonucleotide is selected from F or OMe.

Embodiment 1185. The RNAi agent of embodiment 1184, wherein one G is F and the other G is OMe.

Embodiment 1186. The RNAi agent of any of embodiments 1165 or 1181-1185, wherein the 5′-terminus of the RNAi sense oligonucleotide has structure N:

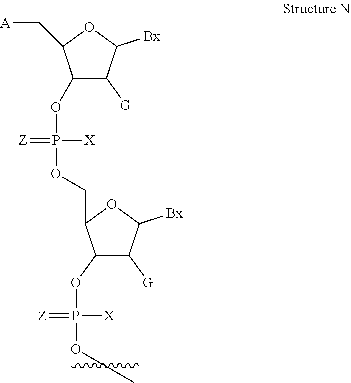

wherein:
A is selected from

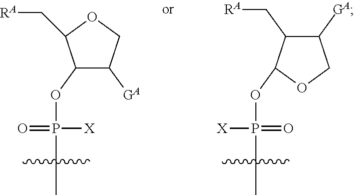

R^{A} is OH, OP(═O)OH, OP(═O)SH, or a stabilized phosphate group;
G^{A} is H, OH, OMe, MOE, or a halogen;
each Bx is an independently selected heterocyclic base moiety;
each X is independently selected from OH or SH;
each G is independently selected from OH, halogen or O—[C(R₆)(R₇)]ₙ[(C═O)ₘ—X^{G}]ⱼ—R₈;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

Embodiment 1187. The RNAi agent of embodiment 1186, wherein each G within structure N of the RNAi sense oligonucleotide is selected from F or OMe.

Embodiment 1188. The RNAi agent of any of embodiments 1165, 1178-1180 or 1183-1187, wherein the 3′-terminus of the RNAi sense oligonucleotide has structure O:

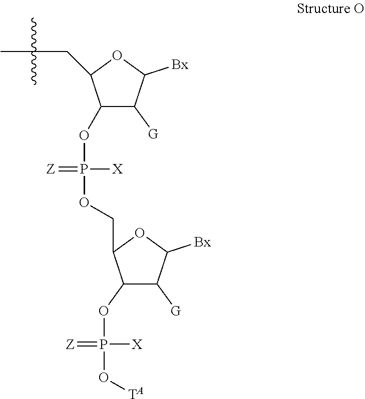

wherein:
T^{A} is selected from

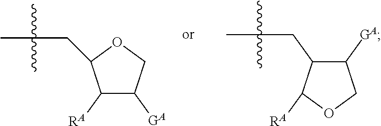

R^{A} is OH, OP(═O)OH, OP(═O)SH, or a stabilized phosphate group;
G^{A} is H, OH, OMe, MOE, or a halogen;
each Bx is an independently selected heterocyclic base moiety;
each X is independently selected from OH or SH;
each Z is selected from O, S, or NSO₂Me;
at least one Z is NSO₂Me;
each G is independently selected from OH, halogen or O—[C(R₆)(R₇)]ₙ—[(C═O)ₘ—X^{G}]ⱼ—R₈;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

Embodiment 1189. The RNAi agent of embodiment 1188, wherein each G within structure O of the RNAi sense oligonucleotide is selected from F or OMe.

Embodiment 1190. The antisense agent of any of embodiments 705-909 or 943-1177, comprising a modified oligonucleotide, wherein the nucleobase sequence of the modified oligonucleotide is complementary to a target nucleic acid.

Embodiment 1191. The modified oligonucleotide of embodiment 1190, wherein the nucleobase sequence of the modified oligonucleotide is at least 80% complementary to the target nucleic acid.

Embodiment 1192. The modified oligonucleotide of embodiment 1190, wherein the nucleobase sequence of the modified oligonucleotide is at least 85% complementary to the target nucleic acid.

Embodiment 1193. The modified oligonucleotide of embodiment 1190, wherein the nucleobase sequence of the modified oligonucleotide is at least 90% complementary to the target nucleic acid.

Embodiment 1194. The modified oligonucleotide of embodiment 1190, wherein the nucleobase sequence of the modified oligonucleotide is at least 95% complementary to the target nucleic acid.

Embodiment 1195. The modified oligonucleotide of embodiment 1190, wherein the nucleobase sequence of the modified oligonucleotide is 100% complementary to the target nucleic acid.

Embodiment 1196. The modified oligonucleotide of any of embodiments 1190-1195, wherein the target nucleic acid is a target RNA.

Embodiment 1197. The modified oligonucleotide of embodiment 1196, wherein the target RNA is selected from: an mRNA, a pre-mRNA, a microRNA, and a non-coding RNA.

Embodiment 1198. The modified oligonucleotide of embodiment 1197, wherein the target RNA is not a microRNA.

Embodiment 1199. The antisense agent comprising a modified oligonucleotide of any of embodiments 1-1198, wherein the modified oligonucleotide is not complementary to miR-21.

Embodiment 1200. The antisense agent of any of embodiments 705-1199, comprising a conjugate group.

Embodiment 1201. The antisense agent of embodiment 1200, wherein the conjugate group comprises at least one GalNAc.

Embodiment 1202. The antisense agent of embodiment 1200 or 1201, wherein the conjugate group comprises 1-5 linker-nucleosides.

Embodiment 1203. A pharmaceutical composition comprising the antisense agent of any of embodiments 705-1202 and a pharmaceutically acceptable carrier or diluent.

Embodiment 1204. A method comprising contacting a cell with the antisense agent or pharmaceutical composition of any of embodiments 705-1203.

Embodiment 1205. A method of modulating the amount or activity of a target nucleic acid in a cell, comprising contacting the cell with the antisense agent or pharmaceutical composition of any of embodiments 705-1204 and thereby modulating the amount or activity of the target nucleic acid.

Embodiment 1206. A method of modulating the amount or activity of a target nucleic acid in a cell, comprising contacting the cell with the antisense agent or pharmaceutical composition of any of embodiments 705-1204.

Embodiment 1207. The method of embodiments 1204-1206, wherein the amount or activity of a target nucleic acid is reduced.

Embodiment 1208. The method of embodiments 1204-1206, wherein the amount or activity of a target nucleic acid is increased.

Embodiment 1209. The method of embodiment 1204, wherein the target protein is encoded by a target nucleic acid comprising at least one translation suppression element and wherein the modified oligonucleotide is complementary to a target site within a translation suppression element region of the target nucleic acid.

Embodiment 1210. The method of embodiment 1209, wherein the translation suppression element region comprises at least one stem-loop structure.

Embodiment 1211. Use of the antisense agent or composition of any of embodiments 705-1203 for treatment of a disease or condition.

Embodiment 1212. Use of the antisense agent or composition of any of embodiments 705-1203 for a preparation of a medicament for treatment of a disease or condition.

Embodiment 1213. The antisense agent of any of embodiments 705-856, 943-1162, or 1190-1202, wherein the antisense agent is not an RNAi agent and the parent antisense agent is cytotoxic in vitro.

Embodiment 1214. The antisense agent of embodiment 1213, wherein the parent antisense agent is cytotoxic in a standard in vitro cytotoxicity assay.

Embodiment 1215. The antisense agent of embodiment 1213, wherein the antisense agent of any of embodiments 705-856, 943-1162, or 1190-1202 is not cytotoxic in vitro.

Embodiment 1216. The antisense agent of any of embodiments 1213-1215, wherein the antisense agent of any of embodiments 705-856, 943-1162, or 1190-1202 is not cytotoxic in a standard in vitro cytoxicity assay.

Embodiment 1217. The antisense agent of any of embodiments 705-856, 943-1162, or 1190-1202, wherein the antisense agent is not an siRNA agent and the parent antisense agent is hepatotoxic to the mouse.

Embodiment 1218. The antisense agent of embodiment 1217, wherein the mouse is a BALB/c mouse, wherein 50 mg/kg of the parent antisense agent is administered to the mouse, and wherein the plasma ALT level in the mouse is measured 72 hours following the administration of the parent antisense agent.

Embodiment 1219. The antisense agent of any of embodiments 1217-1218, wherein administration of 50 mg/kg of the antisense agent of any of embodiments 705-856, 943-1162, or 1190-1202 to a mouse is not hepatotoxic to the mouse.

Embodiment 1220. The antisense agent of any of embodiments 705-856, 943-1162, or 1190-1202, wherein the therapeutic index in a mouse of the antisense agent of any of embodiments 705-856, 943-1162, or 1190-1202 is increased relative to the therapeutic index of the parent antisense agent.

Embodiment 1221. The antisense agent of embodiment 1220, wherein the therapeutic index in a mouse of the antisense agent of embodiment 516 is at least two-fold greater than the therapeutic index of the parent antisense agent.

Embodiment 1222. The antisense agent of any of embodiments 1213-1221, wherein the parent antisense agent is identical to the antisense agent of any of embodiments 705-856, 943-1162, or 1190-1202, except that each internucleoside linkage of Formula XVII is replaced with a phosphorothioate internucleoside linkage in the parent antisense agent.

Embodiment 1223. The antisense agent of any of embodiments 1213-1222, wherein the antisense agent is an RNAse H agent.

Embodiment 1224. The antisense agent of any of embodiments 1213-1222, wherein the antisense agent is a gapmer.

Embodiment 1225. The antisense agent of any of embodiments 1213-1222, wherein the antisense agent modulates splicing.

Embodiment 1226. The antisense agent of any of embodiments 1213-1222, wherein the antisense agent increases protein expression.

Embodiment 1227. The antisense agent of any of embodiments 705-942, 1074-1148 or 1163-1202, wherein the antisense agent is an RNAi agent, and the parent RNAi agent is cytoxic in vitro.

Embodiment 1228. The antisense agent of embodiment 1227, wherein the RNAi agent of any of embodiments 705-942, 1074-1148 or 1163-1202, is not cytotoxic in vitro.

Embodiment 1229. The antisense agent of any of embodiments 1227-1228 wherein the RNAi agent of any of embodiments 705-942, 1074-1148 or 1163-1202 is not cytotoxic in a standard in vitro cytoxicity assay.

Embodiment 1230. The antisense agent of any of embodiments 705-942, 1074-1148 or 1163-1202, wherein the antisense agent is an RNAi agent and is hepatotoxic to the mouse.

Embodiment 1231. The RNAi agent of embodiment 1230, wherein the mouse is a BALB/c mouse, wherein 50 mg/kg of the parent RNAi agent is administered to the mouse, and wherein the plasma ALT level in the mouse is measured 72 hours following the administration of the parent RNAi agent.

Embodiment 1232. The RNAi agent of any of embodiments 1230-1231, wherein administration of 50 mg/kg of the RNAi agent of any of embodiments 705-942, 1074-1148 or 1163-1202 to a mouse is not hepatotoxic to the mouse.

Embodiment 1233. The RNAi agent of any of embodiments 705-942, 1074-1148 or 1163-1202,wherein the therapeutic index in a mouse of the RNAi agent of any of embodiments 705-942, 1074-1148 or 1163-1202 is increased relative to the therapeutic index of the parent RNAi agent.

Embodiment 1234. The RNAi agent of embodiment 1233, wherein the therapeutic index in a mouse of the RNAi agent of embodiment 1233 is at least two-fold greater than the therapeutic index of the parent RNAi agent.

Embodiment 1235. The RNAi agent of any of embodiments 1127-1234, wherein the parent RNAi agent is identical to the RNAi agent of any of embodiments 705-942, 1074-1148 or 1163-1202, except that each internucleoside linkage of Formula XVII is replaced with a phosphodiester internucleoside linkage in the parent RNAi agent.

Embodiment 1236. A method of designing an antisense agent comprising starting with a parent antisense agent or a parent RNAi agent and changing the design of that compound in order to arrive at an antisense agent of any one of embodiments 705-1202.

Embodiment 1237. A method of designing an antisense agent comprising identifying an antisense agent or parent RNAi agent and changing the design of that parent antisense agent or parent RNAi agent to arrive at a second antisense agent, wherein the second antisense agent is an antisense agent of any one of embodiments 705-1202.

Embodiment 1238. A method of improving hepatotoxicity of an antisense agent comprising the steps of (i) identifying a parent antisense agent or parent RNAi agent that has plasma ALT levels above 300 units per liter in a mouse, and (ii) providing an antisense agent according to any one of embodiments 705-1202.

Embodiment 1239. The method of embodiment 1236, wherein the method designs antisense agent with improved therapeutic index relative to the parent antisense agent or parent RNAi agent.

Embodiment 1240. The method of embodiment 1236, wherein the method designs an antisense agent with lower hepatotoxicity relative to the parent antisense agent or parent RNAi agent.

Embodiment 1241. The method of embodiment 1237, wherein the second antisense agent has an improved therapeutic index relative to the parent antisense agent or parent RNAi agent.

Embodiment 1242. The method of embodiment 1237, wherein the second antisense agent has reduced hepatotoxicity in a mouse relative to the parent antisense agent or parent RNAi agent.

Embodiment 1243. The method of embodiment 1238, wherein the antisense agent according to any one of embodiments 705-1202 has improved therapeutic index relative to the parent antisense agent or parent RNAi agent.

Embodiment 1244. The method of embodiment 1238, wherein the antisense agent according to any one of embodiments 705-1202 has reduced hepatotoxicity relative to the parent antisense agent or parent RNAi agent.

Embodiment 1245. A method comprising administering an antisense agent of any of embodiments 705-1202 to a mouse and separately administering the parent antisense agent or parent RNAi agent of the antisense agent of any of embodiments 705-1202 to a second mouse, wherein the therapeutic index of the antisense agent of any of embodiments 705-1202 is improved relative to the therapeutic index of the parent antisense agent or parent RNAi agent.

Embodiment 1246. An oligomeric compound comprising a modified oligonucleotide consisting of 12-70 linked nucleosides linked through internucleoside linking groups, wherein at least one nucleoside comprises a modified sugar moiety, and wherein at least one of the internucleoside linking groups has Formula XVII:

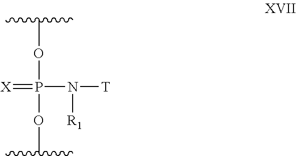

wherein independently for each internucleoside linking group of the modified oligonucleotide having Formula XVII:
   X is selected from O or S;
   R₁ is selected from H, C₁-C₆ alkyl, and substituted C₁-C₆ alkyl; and
   T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group; and
R₅ is selected from OCH₃, OH, C₁-C₆ alkyl, and substituted C₁-C₆ alkyl.

Embodiment 1247. An oligomeric compound comprising a modified oligonucleotide consisting of 12-70 linked nucleosides linked through internucleoside linking groups, wherein at least one nucleoside comprises a modified sugar moiety, and wherein at least one of the internucleoside linking groups has Formula XVII:

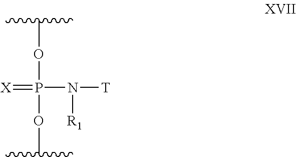

wherein independently for each internucleoside linking group of the modified oligonucleotide having Formula XVII:
   X is selected from O or S;
   R₁ is selected from H, C₁-C₆ alkyl, and substituted C₁-C₆ alkyl; and
   T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group; and
R₅ is selected from OCH₃, OH, C₁-C₆ alkyl, and substituted C₁-C₆ alkyl,
Provided that if X is O and that if R₁ is H, then T is not:

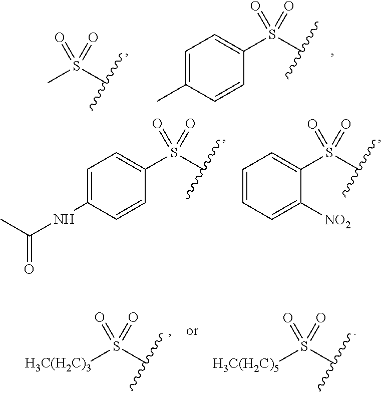

Embodiment 1248. The oligomeric compound of claim 1246 or claim 1247, wherein at least one internucleoside linking group is a phosphodiester or a phosphorothioate internucleoside linking group.

Embodiment 1249. The oligomeric compound of any of claims 1246-1248, wherein at least one nucleoside comprises a 2′-P-D-deoxyribosyl sugar moiety.

Embodiment 1250. The oligomeric compound of any of claims 1246-1249, wherein for at least one internucleoside linking group of Formula XVII, X is O.

Embodiment 1251. The oligomeric compound of any of claims 1246-1250, wherein for at least one internucleoside linking group of Formula XVII, X is S.

Embodiment 1252. The oligomeric compound of claim 1246 or 1247, wherein for at least one internucleoside linking group of Formula XVII, R₁ is H.

Embodiment 1253. The oligomeric compound of claim 1246 or 1247, wherein for at least one internucleoside linking group of Formula XVII, R₁ is a C₁-C₆ alkyl.

Embodiment 1254. The oligomeric compound of claim 1253, wherein R₁ is methyl.

Embodiment 1255. The oligomeric compound of claim 1246 or 1247, wherein for at least one internucleoside linking group of Formula XVII, R₁ is a substituted C₁-C₆ alkyl.

Embodiment 1256. The oligomeric compound of any of claims 1246-1255, wherein for at least one internucleoside linking group of Formula XVII, T comprises a conjugate group.

Embodiment 1257. The oligomeric compound of claim 1256, wherein the conjugate group comprises a cell-targeting moiety.

Embodiment 1258. The oligomeric compound of claim 1256, wherein the conjugate group comprises a carbohydrate or carbohydrate cluster.

Embodiment 1259. The oligomeric compound of any of claims 1256-1258, wherein the conjugate group comprises at least one GalNAc.

Embodiment 1260. The oligomeric compound of claim 1256, wherein the conjugate group comprises a C₁₀-C₂₀ alkyl chain.

Embodiment 1261. The oligomeric compound of claim 1257, wherein the conjugate group comprises C₁₆ alkyl.

Embodiment 1262. The oligomeric compound of any of claims 1246-1255, wherein for at least one internucleoside linking group of Formula XVII, T does not comprise a conjugate group.

Embodiment 1263. The oligomeric compound of any of claims 1246-1255, wherein for at least one internucleoside linking group of Formula XVII, T does not comprise a cell-targeting moiety.

Embodiment 1264. The oligomeric compound of any of claims 1246-1263, wherein for at least one internucleoside linking group of Formula XVII, T is SO₂R₂.

Embodiment 1265. The oligomeric compound of claim 1264, wherein R₂ is an aryl.

Embodiment 1266. The oligomeric compound of claim 1264, wherein R₂ is a substituted aryl.

Embodiment 1267. The oligomeric compound of claim 1264, wherein R₂ is a heterocycle.

Embodiment 1268. The oligomeric compound of claim 1264, wherein R₂ is a substituted heterocycle.

Embodiment 1269. The oligomeric compound of claim 1264, wherein R₂ is an aromatic heterocycle.

Embodiment 1270. The oligomeric compound of claim 1264, wherein R₂ is a substituted aromatic heterocycle.

Embodiment 1271. The oligomeric compound of claim 1264, wherein R₂ is a diazole.

Embodiment 1272. The oligomeric compound of claim 1264, wherein R₂ is a substituted diazole.

Embodiment 1273. The oligomeric compound of claim 1264, wherein R₂ is an amine.

Embodiment 1274. The oligomeric compound of claim 1264, wherein R₂ is a substituted amine.

Embodiment 1275. The oligomeric compound of claim 1264, wherein R₂ is a C₁-C₆ alkoxy, C₁-C₆ alkenyl, or C₁-C₆-alkynl.

Embodiment 1276. The oligomeric compound of claim 1264, wherein R₂ is C₁-C₂0, C₁-C₆, C₂-C₂₀, C₂-C₆, or C₁₀-C₂₀ alkyl.

Embodiment 1277. The oligomeric compound of claim 1264, wherein R₂ is substituted C₁-C₂₀, C₁-C₆, C₂-C₂₀, C₂-C₆, or C₁₀-C₂₀ alkyl.

Embodiment 1278. The oligomeric compound of claim 1264, wherein R₂ comprises a carbohydrate or carbohydrate cluster.

Embodiment 1279. The oligomeric compound of claim 1264, wherein R₂ comprises at least one GalNAc.

Embodiment 1280. The oligomeric compound of claim 1264, wherein T is:

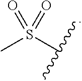 [SMILES: *S(C)(=O)=O]

Embodiment 1281. The oligomeric compund of claim 1264, wherein T is:

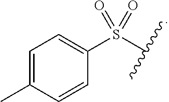 [SMILES: *S(=O)(=O)c1ccc(C)cc1]

Embodiment 1282. The oligomeric compound of claim 1264, wherein T is:

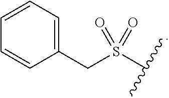 [SMILES: *S(=O)(=O)Cc1ccccc1]

Embodiment 1283. The oligomeric compound of claim 1264, wherein T is:

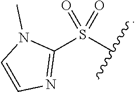 [SMILES: *S(=O)(=O)c1nccn1C]

Embodiment 1284. The oligomeric compound of claim 1264, wherein T is:

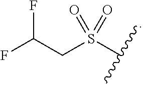 [SMILES: *S(=O)(=O)CC(F)F]

Embodiment 1285. The oligomeric compound of claim 1264, wherein T is:

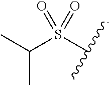 [SMILES: *S(=O)(=O)C(C)C]

Embodiment 1286. The oligomeric compound of claim 1264, wherein T is:

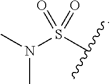 [SMILES: *S(=O)(=O)N(C)C]

Embodiment 1287. The oligomeric compound of claim 1264, wherein T is:

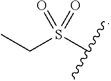 [SMILES: *S(=O)(=O)CC]

Embodiment 1288. The oligomeric compound of claim 1264, wherein T is:

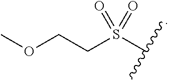 [SMILES: *S(=O)(=O)CCOC]

Embodiment 1289. The oligomeric compound of claim 1264, wherein T is:

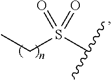

wherein n is from 2 to 20.

Embodiment 1290. The oligomeric compound of claim 1289, wherein n is 15.

Embodiment 1291. The oligomeric compound of any of claims 1246-1290, wherein for at least one internucleoside linking group of Formula XVII, T is C(═O)R₃.

Embodiment 1292. The oligomeric compound of claim 1291, wherein R₃ is an aryl.

Embodiment 1293. The oligomeric compound of claim 1291, wherein R₃ is a substituted aryl.

Embodiment 1294. The oligomeric compound of claim 1291, wherein R₃ is CH₃.

Embodiment 1295. The oligomeric compound of claim 1291, wherein R₃ is N(CH₃)2.

Embodiment 1296. The oligomeric compound of claim 1291, wherein R₃ is OCH₃.

Embodiment 1297. The oligomeric compound of claim 1291, wherein R₃ is a C₁-C₆ alkoxy.

Embodiment 1298. The oligomeric compound of claim 1291, wherein R₃ is C₁-C₂₀, C₁-C₆, C₂-C₂₀, C₂-C₆, or C₁₀-C₂₀ alkyl.

Embodiment 1299. The oligomeric compound of claim 1291, wherein R₃ is substituted C₁-C₂₀, C₁-C₆, C₂-C₂₀, C₂-C₆, or C₁₀-C₂₀ alkyl.

Embodiment 1300. The oligomeric compound of claim 1291, wherein R₃ comprises a carbohydrate or carbohydrate cluster.

Embodiment 1301. The oligomeric compound of claim 1291, wherein R₂₃ comprises at least one GalNAc.

Embodiment 1302. The oligomeric compound of claim 1291, wherein T is:

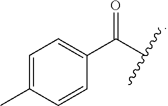 [SMILES: *C(=O)c1ccc(C)cc1]

Embodiment 1303. The oligomeric compound of claim 1291, wherein T is:

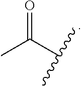 [SMILES: *C(C)=O]

Embodiment 1304. The oligomeric compound of claim 1291, wherein T is:

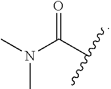 [SMILES: *C(=O)N(C)C]

Embodiment 1305. The oligomeric compound of claim 1291, wherein T is:

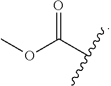 [SMILES: *C(=O)OC]

Embodiment 1306. The oligomeric compound of claim 1291, wherein T is:

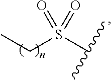

wherein n is from 2 to 20.

Embodiment 1307. The oligomeric compound of claim 1306, wherein n is 15.

Embodiment 1308. The oligomeric compound of any of claims 1246-1263, wherein for at least one internucleoside linking group of Formula XVII, T is P(═O)R₄R₅.

Embodiment 1309. The oligomeric compound of claim 1308, wherein R₄ is OCH₃.

Embodiment 1310. The oligomeric compound of claim 1308, wherein R₄ is OH Embodiment 1311. The oligomeric compound of claim 1308, wherein R₄ is C₁-C₆ alkyl.

Embodiment 1312. The oligomeric compound of claim 1308, wherein R₄ is substituted C₁-C₆ alkyl.

Embodiment 1313. The oligomeric compound of claim 1308, wherein R₄ is C₁-C₂₀, C₁-C₆, C₂-C₂₀, C₂-C₆, or C₁₀-C₂₀ alkyl.

Embodiment 1314. The oligomeric compound of claim 1308, wherein R₄ is substituted C₁-C₂₀, C₁-C₆, C₂-C₂₀, C₂-C₆, or C₁₀-C₂₀ alkyl.

Embodiment 1315. The oligomeric compound of claim 1308, wherein R₄ comprises a carbohydrate or carbohydrate cluster.

Embodiment 1316. The oligomeric compound of claim 1308, wherein R₄ comprises at least one GalNAc.

Embodiment 1317. The oligomeric compound of any of claims 1308-1316, wherein R₅ is OCH₃.

Embodiment 1318. The oligomeric compound of any of claims 1308-1316, wherein R₅ is OH.

Embodiment 1319. The oligomeric compound of any of claims 1308-1316, wherein R₅ is C₁-C₆ alkyl.

Embodiment 1320. The oligomeric compound of any of claims 1308-1316, wherein R₅ is substituted C₁-C₆ alkyl.

Embodiment 1321. The oligomeric compound of claim 1308, wherein T is:

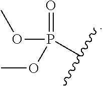 [SMILES: *P(=O)(OC)OC]

Embodiment 1322. The oligomeric compound of claim 1308, wherein T is:

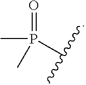 [SMILES: *P(C)(C)=O]

Embodiment 1323. The oligomeric compound of claim 1308, wherein T is:

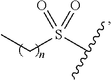

wherein n is from 2 to 20.

Embodiment 1324. The oligomeric compound of claim 1323, wherein n is 15.

Embodiment 1325. The oligomeric compound of any of claims 1246-1324, wherein at least one internucleoside linking group of the modified oligonucleotide is not a linking group of Formula XVII.

Embodiment 1326. The oligomeric compound of any of claims 1246-1325, wherein exactly one internucleoside linking group of the modified oligonucleotide is an internucleoside linking group of Formula XVII.

Embodiment 1327. The oligomeric compound of any of claims 1246-1325, wherein exactly two internucleoside linking groups of the modified oligonucleotide are internucleoside linking groups of Formula XVII.

Embodiment 1328. The oligomeric compound of any of claims 1246-1325, wherein exactly three internucleoside linking groups of the modified oligonucleotide are internucleoside linking groups of Formula XVII.

Embodiment 1329. The oligomeric compound of any of claims 1246-1325, wherein exactly four internucleoside linking groups of the modified oligonucleotide are internucleoside linking groups of Formula XVII.

Embodiment 1330. The oligomeric compound of any of claims 1246-1325, wherein exactly five internucleoside linking groups of the modified oligonucleotide are internucleoside linking groups of Formula XVII.

Embodiment 1331. The oligomeric compound of any of claims 1246-1325, wherein at least six internucleoside linking groups of the modified oligonucleotide are internucleoside linking groups of Formula XVII.

Embodiment 1332. The oligomeric compound of any of claim 1246-1324 or 1326-1331 having at least two linking groups of Formula XVII, wherein at least two of the linking groups of Formula XVII are the same as one another.

Embodiment 1333. The oligomeric compound of any of claims 1246-1332, wherein each internucleoside linking group of the modified oligonucleotide that is not an internucleoside linking group of Formula XVII is either a phosphodiester internucleoside linking group or a phosphorothioate internucleoside linking group.

Embodiment 1334. The oligomeric compound of any of claims 1246-1325 or 1331, wherein each internucleoside linking group of the modified oligonucleotide is an internucleoside linking group of Formula XVII.

Embodiment 1335. An oligomeric compound comprising a modified oligonucleotide, wherein at least one region of the modified oligonucleotide has Structure A:

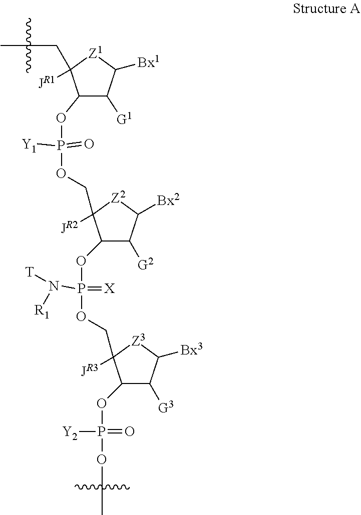

wherein:
each Bx is a heterocyclic base moiety;
X is selected from O or S;
each of Y₁ and Y₂ is independently selected from OH or SH;
each of Z¹, Z², and Z³ are independently selected from —(CH₂)ₚ—X^{Z}—(CH₂)_{q}—, wherein p is 0 or 1, q is 0 or 1, and X^{Z} is O, S, or N(E₁);
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group;
R₅ is selected from OCH₃, OH, C₁-C₆ alkyl, and substituted C₁-C₆ alkyl;
either J^{R1} and G¹ form a J^{R1} to G¹ bridge, or J^{R1} is H and G¹ is selected from H, OH, halogen or O—[C(R₆)(R₇)]ₙ—[(C═O)ₘ—X^{G}]ⱼ—R₈;
either J^{R2} and G² form a J^{R2} and G² bridge, or J^{R2} is H and G² is selected from H, OH, halogen or O—[C(R₆)(R₇)]ₙ—[(C═O)ₘ—X^{G}]ⱼ—R₈;
either J^{R3} and G³ form a J^{R3} and G³ bridge, or J^{R3} is H and G³ is selected from H, OH, halogen or O—[C(R₆)(R₇)]ₙ—[(C═O)ₘ—X^{G}]ⱼ—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂); Q₂ is O, S or NJ₃;
each J₁, J₂ and J₃ is, independently, H or C₁-C₆ alkyl.

Embodiment 1336. An oligomeric compound comprising a modified olignucleotide, wherein at least one region of the modified oligonucleotide has Structure B:

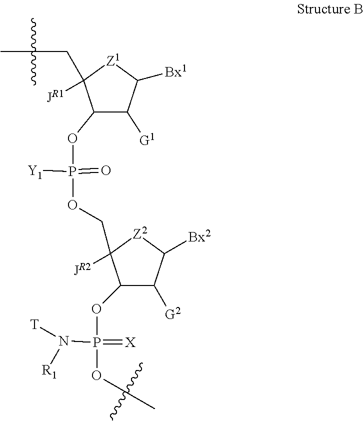

wherein:
   each Bx is a heterocyclic base moiety;
X is selected from O or S;
each of Y₁ and Y₂ is independently selected from OH or SH;
each of Z¹ and Z² are independently selected from —(CH₂)ₚ—X^{Z}—(CH₂)_{q}, wherein p is 0 or 1, q is 0 or 1, and X^{Z} is O, S, or N(E₁);
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group;
R₅ is selected from OCH₃, OH, C₁-C₆ alkyl, and substituted C₁-C₆ alkyl;
either J^{R1} and G¹ form a J^{R1} to G¹ bridge, or J^{R1} is H and G¹ is selected from H, OH, halogen or O—[C(R₆)(R₇)]—[(C═O)ₘ—X^{G}]ⱼ—R₈;
either J^{R2} and G² form a J^{R2} and G² bridge, or J^{R2} is H and G² is selected from H, OH, halogen or O—[C(R₆)(R₇)]ₙ—[(C═O)ₘ—X^{G}]—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂); Q₂ is O, S or NJ₃;
each J₁, J₂ and J₃ is, independently, H or C₁-C₆ alkyl.

Embodiment 1337. An oligomeric compound comprising a modified olignucleotide, wherein at least one region of the modified oligonucleotide has Structure C:

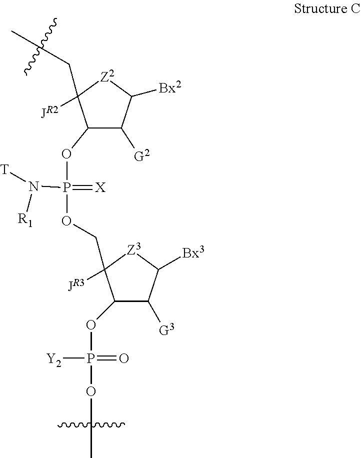

wherein:
each Bx is a heterocyclic base moiety;
X is selected from O or S;
each of Y₁ and Y₂ is independently selected from OH or SH;
each of Z² and Z³ are independently selected from —(CH₂)ₚ—X^{Z}—(CH₂)_{q}—, wherein p is 0 or 1, q is 0 or 1, and X^{Z} is O, S, or N(E₁);
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group;
R₅ is selected from OCH₃, OH, C₁-C₆ alkyl, and substituted C₁-C₆ alkyl;
   either J^{R}₂ and G² form a J^{R}₂ and G² bridge, or J^{R}₂ is H and G² is selected from H, OH, halogen or O—[C(R₆)(R₇)]ₙ—[(C═O)ₘ—X^{G}]—R₈;
either J^{R3} and G³ form a J^{R3} and G³ bridge, or J^{R3} is H and G³ is selected from H, OH, halogen or O—[C(R₆)(R₇)]ₙ—[(C═O)ₘ—X^{G}]—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

Embodiment 1338. An oligomeric compound comprising a modified olignucleotide, wherein at least one region of the modified oligonucleotide has Structure D:

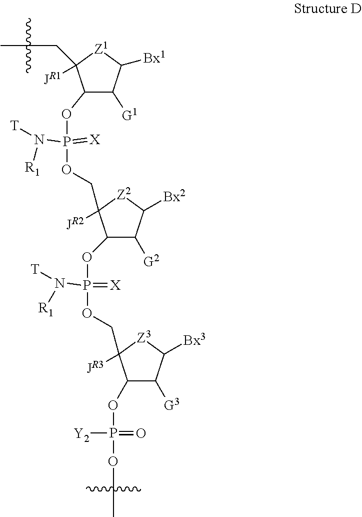

wherein:
each Bx is a heterocyclic base moiety;
X is selected from O or S;
each of Y₁ and Y₂ is independently selected from OH or SH;
each of Z² and Z³ are independently selected from —(CH₂)ₚ—X^{Z}—(CH₂)_{q}—, wherein p is 0 or 1, q is 0 or 1, and X^{Z} is O, S, or N(E₁);
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group;
R₅ is selected from OCH₃, OH, C₁-C₆ alkyl, and substituted C₁-C₆ alkyl;
either J^{R1} and G¹ form a J^{R1} to G¹ bridge, or J^{R1} is H and G¹ is selected from H, OH, halogen or O—[C(R₆)(R₇)]—[(C═O)ₘ—X^{G}]j-R₈;
either J^{R2} and G² form a J^{R2} and G² bridge, or J^{R2} is H and G² is selected from H, OH, halogen or O—[C(R₆)(R₇)]ₙ—[(C═O)ₘ—X^{G}]ⱼ—R₈;
either J^{R3} and G³ form a J^{R3} and G³ bridge, or J^{R3} is H and G³ is selected from H, OH, halogen or O—[C(R₆)(R₇)]ₙ—[(C═O)ₘ—X^{G}]—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

Embodiment 1339. An oligomeric compound comprising a modified olignucleotide, wherein at least one region of the modified oligonucleotide has Structure E:

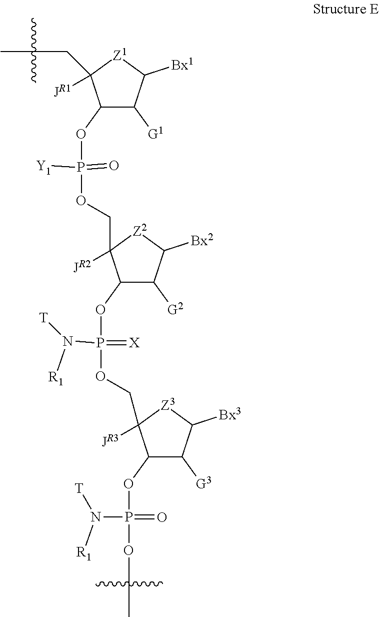

wherein:
each Bx is a heterocyclic base moiety;
X is selected from O or S;
each of Y₁ and Y₂ is independently selected from OH or SH;
each of Z² and Z³ are independently selected from —(CH₂)ₚ—X^{Z}—(CH₂)_{q}—, wherein p is 0 or 1, q is 0 or 1, and X^{Z} is O, S, or N(E₁);
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group;
R₅ is selected from OCH₃, OH, C₁-C₆ alkyl, and substituted C₁-C₆ alkyl;
either J^{R1} and G¹ form a J^{R1} to G¹ bridge, or J^{R1} is H and G¹ is selected from H, OH, halogen or O—[C(R₆)(R₇)]—[(C═O)ₘ—X^{G}]ⱼ—R₈;
either J^{R2} and G² form a J^{R2} and G² bridge, or J^{R2} is H and G² is selected from H, OH, halogen or O—[C(R₆)(R₇)]ₙ—[(C═O)ₘ—X^{G}]ⱼ—R₈;
either J^{R3} and G³ form a J^{R3} and G³ bridge, or J^{R3} is H and G³ is selected from H, OH, halogen or O—[C(R₆)(R₇)]ₙ—[(C═O)ₘ—X^{G}]ⱼ—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

Embodiment 1340. An oligomeric compound comprising a modified oligonucleotide, wherein the 5′-terminus of the modified oligonucleotide has structure P:

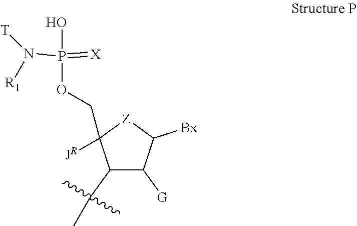

wherein:
each Bx is a heterocyclic base moiety;
X is selected from O or S;
Z is —(CH₂)ₚ—X^{Z}—(CH₂)_{q}—, wherein p is 0 or 1, q is 0 or 1, and X^{Z} is O, S, or N(E₁);
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group;
R₅ is selected from OCH₃, OH, C₁-C₆ alkyl, and substituted C₁-C₆ alkyl;
either J^{R} and G form a J^{R} to G bridge, or J^{R} is H and G is selected from H, OH, halogen or O—[C(R₆)(R₇)]—[(C═O)ₘ—X^{G}]ⱼ—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X2)N(J₁)(J₂) and C(=Q₂)N(J₁)(J₂);
Q₂ is O, S or NJ₃;
each J₁, J₂ and J₃ is, independently, H or C₁-C₆ alkyl.

Embodiment 1341. The oligomeric compound of any of claims 1335-1340, wherein each Z is O.

Embodiment 1342. The oligomeric compound of any of claims 1335-1341, wherein at least one G is selected from H, OH, halogen, C₁-C₆ alkoxy, —O(CH₂)₂OCH₃, or —OCH₂(C═O)NHCH₃.

Embodiment 1343. The oligomeric compound of any of claims 1335-1342, wherein each G is selected from H, OH, halogen, C₁-C₆ alkoxy, —O(CH₂)₂OCH₃, or —OCH₂(C═O)NHCH₃.

Embodiment 1344. The oligomeric compound of any of claims 1335-1343, wherein at least one J^{R} forms a bridge with at least one G, wherein said J^{R} to G bridge has a formula selected from —CH(CH₃)—O— or —(CH₂)ₖ—O′, wherein k is from 1 to 3.

Embodiment 1345. The oligomeric compound of any of claims 1335-1342 or 1344, wherein each J^{R} and G form a bridge, wherein said J^{R} to G bridge has a formula selected from —CH(CH₃)—O— or —(CH₂)ₖ—O—, wherein k is from 1 to 3.

Embodiment 1346. The oligomeric compound of any of claims 1344 or 1345, wherein at least one Z is 0 and the corresponding J^{R} to G bridge has a formula (CH₂)ₖ—O—, wherein k is 1.

Embodiment 1347. The oligomeric compound of any of claims 1335-1346 wherein each nucleoside of structure A, B, C, D, E, or P is a stereo standard nucleoside.

Embodiment 1348. The oligomeric compound of any of claims 1335-1346, wherein at least one nucleoside of structure A, B, C, D, E or P is a stereo-non-standard nucleoside.

Embodiment 1349. The oligomeric compound of any of claims 1344-1346 or 1348, wherein at least one nucleoside having a J^{R} to G bridge is in the α-L-ribosyl configuration.

Embodiment 1350. The oligomeric compound of any of claims 1335-1349, wherein the modified oligonucleotide comprises at least 2, at least 3, at least 4, at least 5, at least 6, at least 7, at least 8, at least 9, or at least 10 regions having structures A, B, C, D, or E.

Embodiment 1351. The oligomeric compound of any of claims 1335-1350, wherein at least one region having structure A, B, C, D, or E is at the 5′ end of the modified oligonucleotide.

Embodiment 1352. The oligomeric compound of any of claims 1335-1350, wherein at least one region having structure A, B, C, D, or E is at the 3′ end of the modified oligonucleotide.

Embodiment 1353. The oligomeric compound of any of claims 1335-1350, wherein at least one region having structure A, B, C, D, or E is internal to the modified oligonucleotide.

Embodiment 1354. An oligomeric compound comprising a modified oligonucleotide consisting of 10-30 linked nucleosides, wherein a region of the modified oligonucleotide has the formula (N_{g1})_{L}1(N_{g2})_{L2}(N_{g3})_{L3}, wherein each N_{g} is a nucleoside and each L is an internucleoside linking group; wherein each of L₁, and L₂ is a phosphodiester internucleoside linking group, a phosphorothioate internucleoside linking group, or an internucleoside linking group of Formula XVII:

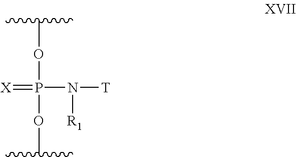

wherein L₃ is absent or is a phosphodiester internucleoside linking group, a phosphorothioate internucleoside linking group, or an internucleoside linking group of Formula XVII;
wherein at least one of L₁, L₂, and L₃ is an internucleoside linking group of Formula XVII; and at least one of L₁, L₂, and L₃ is a phosphorothioate or a phosphodiester internucleoside linking group, wherein independently for each internucleoside linking group of the modified oligonucleotide having Formula XVII:
X is selected from O or S;
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate;
R₄ is selected from OCH₃, OH, C₁-C₆ alkyl, substituted C₁-C₆ alkyl and a conjugate; and
R₅ is selected from OCH₃, OH, C₁-C₆ alkyl, and substituted C₁-C₆ alkyl.

Embodiment 1355. The oligomeric compound of claim 1354, wherein the modified oligonucleotide comprises at least 2, at least 3, at least 4, at least 5, at least 6, at least 7, at least 8, at least 9, or at least 10 regions having the formula (N_{g1})_{L1}(N_{g2})_{L2}(N_{g3})_{L3}.

Embodiment 1356. The oligomeric compound of claim 1354 or 1355, wherein at least one region having the formula (N_{g1})_{L1}(N_{g2})_{L2}(N_{g3})_{L3} is at the 5′ end of the oligonucleotide

Embodiment 1357. The oligomeric compound of claim 1354 or 1355, wherein at least one region having the formula (N_{g1})_{L1}(N_{g2})_{L2}(N_{g3})_{L3} is internal to the oligonucleotide.

Embodiment 1358. The oligomeric compound of claim 1354 or 1355, wherein at least one region having the formula (N_{g1})_{L1}(N_{g2})_{L2}(N_{g3})_{L3} is at the 3′ end of the oligonucleotide.

Embodiment 1359. The oligomeric compound of any of claims 1246-1358, wherein at least one nucleoside of the modified oligonucleotide is a modified nucleoside selected from a bicyclic nucleoside and a non-bicyclic substituted nucleoside.

Embodiment 1360. The oligomeric compound of any of claims 1246-1359, wherein at least one nucleoside of the modified oligonucleotide is selected from: a β-D-LNA nucleoside, an α-L-LNA nucleoside, an ENA nucleoside, a cEt nucleoside, a 2′-MOE nucleoside, a 2′-OMe nucleoside, a 2′-F nucleoside, a 2′-NMA nucleoside, a 5′-Me nucleoside, a DNA nucleoside, and an RNA nucleoside.

Embodiment 1361. The oligomeric compound of any of claims 1246-1360, wherein each nucleoside of the modified oligonucleotide is selected from: a β-D-LNA nucleoside, an α-L-LNA nucleoside, an ENA nucleoside, a cEt nucleoside, a 2′-MOE nucleoside, a 2′-OMe nucleoside, a 2′-F nucleoside, a 2′-NMA nucleoside, a 5′-Me nucleoside, a DNA nucleoside, and an RNA nucleoside.

Embodiment 1362. The oligomeric compound of any of claims 1246-1361, wherein at least one nucleoside of the modified oligonucleotide is a stereo-non-standard nucleoside.

Embodiment 1363. The oligomeric compound of claim 1362, wherein the internucleoside linking group linking at least one stereo-non-standard nucleoside to an adjacent nucleoside is an internucleoside linking group of Formula XVII.

Embodiment 1364. The oligomeric compound of claim 1362 or 1363, wherein at least two nucleosides of the modified oligonucleotide are stereo-non-standard nucleosides.

Embodiment 1365. The oligomeric compound of claim 1364, wherein at least two stereo-non-standard nucleosides of the modified oligonucleotide are adjacent to one another.

Embodiment 1366. The oligomeric compound of claim 1365, wherein at least two stereo-non-standard nucleosides of the modified oligonucleotide are linked to one another with an internucleoside linking group of Formula XVII.

Embodiment 1367. The oligomeric compound of any of claims 1362-1367, wherein at least one stereo-non-standard nucleoside of the modified oligonucleotide is a stereo-non-standard DNA nucleoside.

Embodiment 1368. The oligomeric compound of claim 1367, wherein the stereo-non-standard DNA nucleoside is selected from a stereo-non-standard DNA nucleoside having: Formula I, Formula II, Formula III, Formula IV, Formula V, Formula VI, and Formula VII.

Embodiment 1369. The oligomeric compound of claim 1368 wherein the stereo-non-standard DNA nucleoside is selected from a stereo-non-standard DNA nucleoside having: Formula V and Formula II.

Embodiment 1370. The oligomeric compound of any of claims 1362-1369, wherein at least one stereo-non-standard nucleoside of the oligomeric compound is a substituted stereo-non-standard nucleoside.

Embodiment 1371. The oligomeric compound of claim 1370, wherein the 2′-substituent of the at least one substituted stereo-non-standard nucleoside of the modified oligonucleotide is selected from: 2′-MOE, 2′-OMe, 2′-F, or 2′-OH.

Embodiment 1372. The oligomeric compound of any of claims 1246-1371, wherein each nucleoside of the modified oligonucleotide is a stereo-standard nucleoside.

Embodiment 1373. The oligomeric compound of any of claims 1246-1371, wherein the modified oligonucleotide consists of 12-30 linked nucleosides.

Embodiment 1374. The oligomeric compound of any of claims 1246-1371, wherein the modified oligonucleotide consists of 16-24 linked nucleosides.

Embodiment 1375. The oligomeric compound of any of claims 1246-1371, wherein the modified oligonucleotide consists of 18-22 linked nucleosides.

Embodiment 1376. The oligomeric compound of any of claims 1246-1374, wherein the modified oligonucleotide consists of 16 linked nucleosides.

Embodiment 1377. The oligomeric compound of any of claims 1246-1374, wherein the modified oligonucleotide consists of 17 linked nucleosides.

Embodiment 1378. The oligomeric compound of any of claims 1246-1375, wherein the modified oligonucleotide consists of 18 linked nucleosides.

Embodiment 1379. The oligomeric compound of any of claims 1246-1375, wherein the modified oligonucleotide consists of 19 linked nucleosides.

Embodiment 1380. The oligomeric compound of any of claims 1246-1375, wherein the modified oligonucleotide consists of 20 linked nucleosides.

Embodiment 1381. The oligomeric compound of any of claims 1246-1375, wherein the modified oligonucleotide consists of 21 linked nucleosides.

Embodiment 1382. The oligomeric compound of any of claims 1246-1375, wherein the modified oligonucleotide consists of 22 linked nucleosides.

Embodiment 1383. The oligomeric compound of any of claims 1246-1374, wherein the modified oligonucleotide consists of 23 linked nucleosides.

Embodiment 1384. The oligomeric compound of any of claims 1246-1383, wherein at least one nucleoside of the modified oligonucleotide is selected from: a 2′-OMe nucleoside, a 2′-F nucleoside, and an RNA nucleoside.

Embodiment 1385. The oligomeric compound of any of claims 1246-139, wherein at least one nucleoside of the modified oligonucleotide is a 2′-OMe nucleoside, and at least one nucleoside of the modified oligonucleotide is a 2′-F nucleoside.

Embodiment 1386. The oligomeric compound of claim 1385, wherein each nucleoside of the modified oligonucleotide is selected from a 2′-OMe nucleoside or a 2′-F nucleoside.

Embodiment 1387. The oligomeric compound of any of claims 1246-1385, wherein at least one nucleoside of the modified oligonucleotide is a 2′-OMe nucleoside, at least one nucleoside of the modified oligonucleotide is a 2′-F nucleoside, and at least one nucleoside of the modified oligonucleotide comprises a sugar surrogate.

Embodiment 1388. The oligomeric compound of claim 1387, wherein each nucleoside of the modified oligonucleotide is selected from a 2′-OMe nucleoside, a 2′-F nucleoside, and a nucleoside comprising a sugar surrogate.

Embodiment 1389. The oligomeric compound of any of claims 1387-1388, wherein the nucleoside comprising a sugar surrogate is selected from:

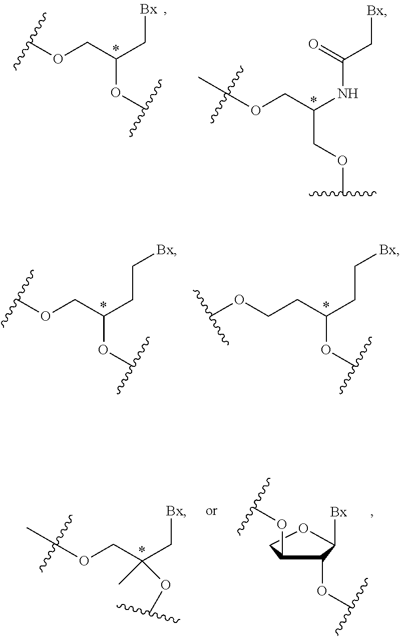

wherein Bx is a heterocyclic base moiety.

Embodiment 1390. The oligomeric compound of claim 1389, wherein the nucleoside comprising a sugar surrogate is GNA.

Embodiment 1391. The oligomeric compound of any of claims 1384-1390, wherein the modified oligonucleotide has a region of alternating nucleoside types having the motif ABABA, wherein each A is a stereo-standard nucleoside of a first type and each B is a stereo-standard nucleoside of a second type, wherein the first type and the second type are different from one another.

Embodiment 1392. The oligomeric compound of claim 1391, wherein A and B are selected from 2′-F substituted nucleosides, 2′-OMe substituted nucleosides, and stereo-standard RNA nucleosides.

Embodiment 1393. The oligomeric compound of any of claims 1246-1392, wherein the 5′-end of the modified oligonucleotide comprises a terminal group.

Embodiment 1394. The oligomeric compound of claim 1393, wherein the terminal group is a stabilized phosphate group.

Embodiment 1395. The oligomeric compound of claim 1394, wherein the stabilized phosphate group is a 5′-vinyl phosphonate or a 5′-cyclopropyl phosphonate.

Embodiment 1396. The compound of claim 1393, wherein the terminal group has Formula XXII:

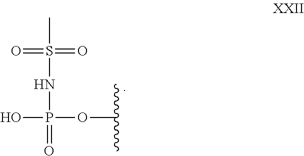 [SMILES: *OP(=O)(O)NS(C)(=O)=O]

Embodiment 1397. The oligomeric compound of claim 1393, wherein the terminal group is selected from

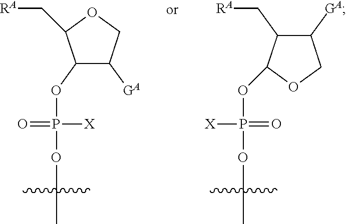

wherein R^{A} is OH, OP(═O)OH, OP(═O)SH, a mesyl phosphoramidate, or a stabilized phosphate group;
G^{A} is H, OH, OMe, MOE, or a halogen;
X is OH, SH, or NSO₂R₂;
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group.

Embodiment 1398. The oligomeric compound of claim 1397, wherein G^{A} is selected from H or OH and X is SH.

Embodiment 1399. An antisense agent consisting or comprising an oligomeric compound of any of claims 1246-1398.

Embodiment 1400. The antisense agent of claim 1399, wherein the antisense agent is an RNAi agent.

Embodiment 1401. The RNAi agent of claim 1400, wherein the RNAi agent is a single-stranded RNAi agent comprising an RNAi antisense oligomeric compound, wherein the RNAi antisense oligomeric compound is an oligomeric compound of any of claims 1246-1398.

Embodiment 1402. The RNAi agent of claim 1401, wherein the RNAi agent is an oligonucleotide duplex comprising an RNAi antisense oligomeric compound and an RNAi sense oligomeric compound, wherein the RNAi antisense oligomeric compound and/or the RNAi sense oligomeric compound is an oligomeric compound of any of claims 1-153.

Embodiment 1403. The RNAi agent of claim 1401 or 1402, wherein at least one internucleoside linking group of the RNAi antisense oligomeric compound is an internucleoside linking group of Formula XVII.

Embodiment 1404. The RNAi agent of claim 1401 or 1402, wherein at least two internucleoside linking groups of the RNAi antisense oligomeric compound are independently selected internucleoside linking groups of Formula XVII.

Embodiment 1405. The RNAi agent of any of claims 1401-1404, wherein at least one of the five 3′-most internucleoside linking groups of the RNAi antisense oligomeric compound is an internucleoside linking group of Formula XVII.

Embodiment 1406. The RNAi agent of any of claims 1401-1404, wherein at least two of the five 3′-most internucleoside linking groups of RNAi antisense oligomeric compound is an internucleoside linking group of Formula XVII.

Embodiment 1407. The RNAi agent of any of claims 1401-1406, wherein 1-3 of the three 3′-most internucleoside linking groups are internucleoside linking groups of Formula XVII, and each of these three internucleoside linking groups that is not an internucleoside linking group of Formula XVII is a phosphodiester or phosphorothioate internucleoside linking group.

Embodiment 1408. The RNAi agent of claim 1407, wherein the two 3′-most internucleoside linking groups are internucleoside linking groups of Formula XVII.

Embodiment 1409. The RNAi agent of any of claims 1401-1408, wherein exactly one of the 5′-most and penultimate 5′-most internucleoside linking groups is an internucleoside linking group of Formula XVII.

Embodiment 1410. The RNAi agent of any of claims 1401-1409, wherein exactly one of the 5′-most and penultimate 5′-most internucleoside linking groups of the RNAi antisense oligonucleotide is an internucleoside linking groups of Formula XVII, the other of the 5′-most and penultimate 5′-most internucleoside linking groups of the RNAi antisense oligonucleotide is selected from a phosphodiester and a phosphorothioate internucleoside linkage, the two 3′-most internucleoside linking groups of the RNAi antisense oligonucleotide are internucleoside linking groups of Formula XVII, and the remaining internucleoside linking groups of the RNAi antisense oligonucleotide are phosphodiester internucleoside linkages.

Embodiment 1411. The RNAi agent of any of claims 1401-1410, wherein the antisense oligomeric compound comprises a 3′-overhang.

Embodiment 1412. The RNAi agent of claim 1411, wherein the 3′-overhang consists of two nucleosides.

Embodiment 1413. The RNAi agent of any of claims 1401-1409 or 1411-1412, wherein at least one internucleoside linking group within the seed region of the RNAi antisense oligomeric compound is an internucleoside linking group of Formula XVII.

Embodiment 1414. The RNAi agent of any of claims 1401-1413, wherein for each internucleoside linking group of Formula XVII, R₁ is H and T is SO₂Me.

Embodiment 1415. The RNAi agent of any of claims 1401-1414, wherein the RNAi antisense oligomeric compound comprises an RNAi antisense modified oligonucleotide, wherein the RNAi antisense modified oligonucleotide consists of 23 linked nucleosides, and the internucleoside linkage motif is selected from: ooooooooooooooooooooaa, aaoooooooooooooooooooo, aaooooooooooooooooooaa, asooooooooooooooooooss, saoooooooooooooooooooo, oooooooooooooooooooaaa, ooooooooooooooooaaaoss, oooooooooooooaaaooooss, ooooooooooaaaoooooooss, oooooooaaaooooooooooss, ooooaaaoooooooooooooss, saoooaoooooooaoaooooss, ssoooaoooooooaoaooooss, or ssooooooooooooooooooaa, wherein each “a” represents an internucleoside linkage of Formula XVII, each “s” represents a phosphorothioate internucleoside linkage, and each “o” represents a phosphodiester internucleoside linkage.

Embodiment 1416. The RNAi agent of claim 1415, wherein the internucleoside linkage motif of the RNAi antisense modified oligonucleotide is selected from oooooooooooooooooooaa, asooooooooooooooooooss, or saoooooooooooooooooooo.

Embodiment 1417. The RNAi agent of claim 1415 or 1416, wherein the sugar motif of the RNAi antisense oligomeric compound from 5′ to 3′ is yfyfyfyfyfyfyfyfyfyfyfy or yfyyyfyyyyyyyfyfyyyyyyy, wherein “y” represents a 2′-OMe sugar moiety and “f” represents a 2′-F sugar moiety.

Embodiment 1418. The RNAi agent of any of claims 1401-1414, wherein the RNAi antisense oligomeric compound comprises an RNAi antisense modified oligonucleotide, wherein the RNAi antisense modified oligonucleotide consists of 21 linked nucleosides, and the internucleoside linkage motif is selected from: aaososososososssssss, ssaaosososososssssss, ssosaaososososssssss, ssososaaosososssssss, ssosososaaososssssss, ssososososaaosssssss, ssosososososaassssss, ssososososososaassss, ssososososososssaass, ssososososososssssaa wherein each “a” represents an internucleoside linkage of Formula XVII, each “s” represents a phosphorothioate internucleoside linkage, and each “o” represents a phosphodiester internucleoside linkage.

Embodiment 1419. The RNAi agent of claim 1418, wherein the internucleoside linkage motif of the RNAi antisense modified oligonucleotide is selected from aaososososososssssss, ssaaosososososssssss, or ssososososososssssaa, wherein each “a” represents an internucleoside linkage of Formula XVII, each “s” represents a phosphorothioate internucleoside linkage, and each “o” represents a phosphodiester internucleoside linkage.

Embodiment 1420. The RNAi agent of claim 1418 or 1419, wherein the sugar motif of the RNAi antisense oligomeric compound from 5′ to 3′ is yfyfyfyfyfyfyfyfyfyfyfy, wherein “y” represents a 2′-OMe sugar moiety and “f” represents a 2′-F sugar moiety.

Embodiment 1421. The RNAi agent of any of claims 1415-1420 wherein each “a” is a mesyl phosphoramidate linkage.

Embodiment 1422. The RNAi agent of any of claims 1401-1421, wherein at least one region of the RNAi antisense oligomeric compound has structure A, B, C, D, E, or P.

Embodiment 1423. The RNAi agent of claim 1422, wherein at least one region having structure A, B, C, D, or E is within the seed region of the RNAi antisense oligomeric compound.

Embodiment 1424. The RNAi agent of claim 1422, wherein at least one region having structure A, B, C, D, or E is at the 3′ end of the RNAi antisense oligomeric compound.

Embodiment 1425. The RNAi agent of claim 1422, wherein at least one region having structure A, B, C, D, E, or P is at the 5′ end of the RNAi antisense oligomeric compound.

Embodiment 1426. The RNAi agent of any of claims 1401-1425, wherein at least one region of the RNAi antisense oligomeric compound has the formula (N_{g1})_{L1}(N_{g2})_{L2}(N_{g3})_{L3}, wherein each N_{g} is a nucleoside and each L is an internucleoside linking group; wherein each of L₁, and L₂ is a phosphodiester internucleoside linking group, a phosphorothioate internucleoside linking group, or an internucleoside linking group of Formula XVII:

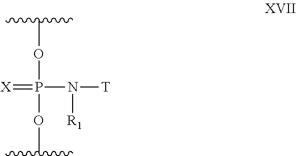

wherein L₃ is absent or is a phosphodiester internucleoside linking group, a phosphorothioate internucleoside linking group, or an internucleoside linking group of Formula XVII;
wherein at least one of L₁, L₂, and L₃ an internucleoside linking group of Formula XVII; and at least one of L₁, L₂, and L₃ is a phosphorothioate or a phosphodiester internucleoside linking group, wherein independently for each internucleoside linking group of the RNAi antisense oligomeric compound having Formula XVII:
X is selected from O or S;
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, substituted C₁-C₆ alkyl, and a conjugate;
R₃ is selected from an aryl, a substituted aryl, CH₃, N(CH₃)₂, OCH₃ and a conjugate;
R₄ is selected from OCH₃, OH, C₁-C₆ alkyl, substituted C₁-C₆ alkyl and a conjugate; and
R₅ is selected from OCH₃, OH, C₁-C₆ alkyl, and substituted C₁-C₆ alkyl.

Embodiment 1427. The RNAi agent of claim 1426, wherein the region having the formula (N_{g1})_{L1}(N_{g2})_{L2}(N_{g3})_{L3} includes one or two 3′-overhang nucleosides.

Embodiment 1428. The RNAi agent of claim 1426, wherein at least one region having the formula (N_{g1})_{L1}(N_{g2})_{L2}(N_{g3})L₃ is at the 3′ end of the RNAi antisense oligomeric compound.

Embodiment 1429. The RNAi agent of claim 1428, wherein L 1 and L2 are each internucleoside linkages of Formula XVII wherein R₁ is H and T is SO₂Me, and L3 is a phosphodiester internucleoside linkage.

Embodiment 1430. The RNAi agent of claim 1428, wherein at least one region having the formula (N_{g1})_{L1}(N_{g2})_{L2}(N_{g3})L₃ is at the 5′ end of the RNAi antisense oligomeric compound.

Embodiment 1431. The RNAi agent of claim 1429, wherein one of L 1 or L2 is an internucleoside linkages of Formula XVII wherein R₁ is H and T is SO₂Me, the other of L 1 or L2 is a phosphorothioate internucleoside linkage, and L3 is a phosphodiester internucleoside linkage.

Embodiment 1432. The RNAi agent of claim 1426, wherein at least one region having the formula (N_{g1})_{L1}(N_{g2})_{L2}(N_{g3})L₃ is within the seed region of the RNAi antisense oligomeric compound.

Embodiment 1433. The RNAi agent of any of claims 1401-1432, wherein the region of the RNAi antisense oligonucleotide that is complementary to a target is at least 15 nucleobases.

Embodiment 1434. The RNAi agent of any of claims 1401-1433, wherein the region of the RNAi antisense oligonucleotide that is complementary to a target is at least 17 nucleobases.

Embodiment 1435. The RNAi agent of any of claims 1401-1434, wherein the region of the RNAi antisense oligonucleotide that is complementary to a target is at least 19 nucleobases.

Embodiment 1436. The RNAi agent of any of claims 1401-1435, wherein the region of the RNAi antisense oligonucleotide that is complementary to a target is at least 21 nucleobases.

Embodiment 1437. The RNAi agent of any of claims 1401-1435, wherein the region of the RNAi antisense oligonucleotide that is complementary to a target is exactly 19 nucleobases.

Embodiment 1438. The RNAi agent of any of claims 1401-1436, wherein the region of the RNAi antisense oligonucleotide that is complementary to a target is exactly 21 nucleobases.

Embodiment 1439. The RNAi agent of any of claims 1401-1438, wherein at least one nucleoside of the RNAi antisense oligomeric compound is selected from: a 2′-OMe nucleoside, a 2′-F nucleoside, and an RNA nucleoside.

Embodiment 1440. The RNAi agent of any of claims 1401-1439, wherein at least one nucleoside of the RNAi antisense oligomeric compound is a 2′-OMe nucleoside, and at least one nucleoside of the RNAi antisense oligomeric compound is an RNA nucleoside.

Embodiment 1441. The RNAi agent of any of claims 1401-1440, wherein at least one nucleoside of the RNAi antisense oligomeric compound is a 2′-OMe nucleoside, and at least one nucleoside of the RNAi antisense oligomeric compound is a 2′-F nucleoside.

Embodiment 1442. The RNAi agent of claim 1441, wherein each nucleoside of the RNAi antisense oligomeric compound is selected from a 2′-OMe nucleoside or a 2′-F nucleoside.

Embodiment 1443. The RNAi agent of any of claims 1401-1439, wherein at least one nucleoside of the RNAi antisense oligomeric compound is a 2′-OMe nucleoside, at least one nucleoside of the RNAi antisense oligomeric compound is a 2′-F nucleoside, and at least one nucleoside of the oligomeric compound comprises a sugar surrogate.

Embodiment 1444. The RNAi agent of claim 1443, wherein each nucleoside of the RNAi antisense oligomeric compound is selected from a 2′-OMe nucleoside, a 2′-F nucleoside, and a nucleoside comprising a sugar surrogate.

Embodiment 1445. The RNAi agent of any of claims 1443-1444, wherein the nucleoside comprising a sugar surrogate is selected from:

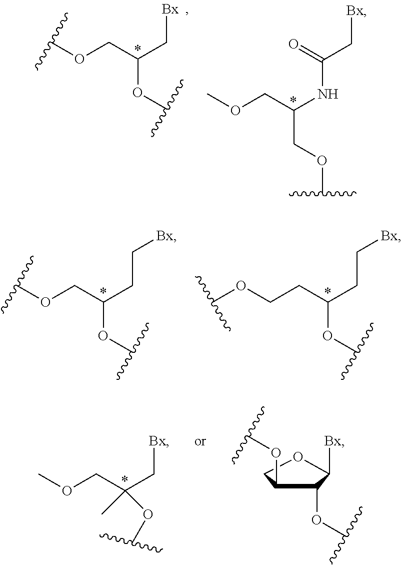

wherein Bx is a heterocyclic base moiety.

Embodiment 1446. The RNAi agent of claim 1445, wherein the nucleoside comprising a sugar surrogate is GNA.

Embodiment 1447. The RNAi agent of claim 1445 or 1446, wherein at least one nucleoside comprising a sugar surrogate is one of the nine 5′-most nucleosides of the RNAi antisense oligomeric compound.

Embodiment 1448. The RNAi agent of any of claims 1401-1447, wherein the oligomeric compound has a region of alternating nucleoside types having the motif ABABA, wherein each A is a stereo-standard nucleoside of a first type and each B is a stereo-standard nucleoside of a second type, wherein the first type and the second type are different from one another.

Embodiment 1449. The RNAi agent of claim 1448, wherein A and B are selected from 2′-F substituted nucleosides, 2′-OMe substituted nucleosides, and stereo-standard RNA nucleosides.

Embodiment 1450. The RNAi agent of any of claims 1401-1449, wherein the 5′-end of the RNAi antisense oligomeric compound comprises a terminal group.

Embodiment 1451. The RNAi agent of claim 1450, wherein the terminal group is a stabilized phosphate group.

Embodiment 1452. The RNAi agent of claim 1451, wherein the stabilized phosphate group is a 5′-vinyl phosphonate or a 5′-cyclopropyl phosphonate.

Embodiment 1453. The RNAi agent of claim 1542, wherein the terminal group has Formula XXII:

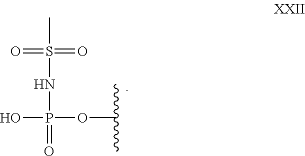 [SMILES: *OP(=O)(O)NS(C)(=O)=O]

Embodiment 1454. The RNAi agent of claim 1450, wherein the terminal group is selected from:

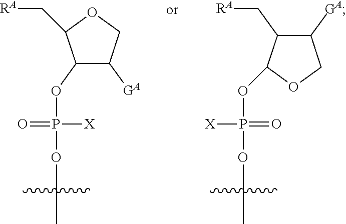

wherein R^{A} is OH, OP(═O)OH, OP(═O)SH, a mesyl phosphoramidate, or a stabilized phosphate group; G^{A} is H, OH, OMe, MOE, or a halogen;
X is OH, SH, or NSO₂R₂;
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group.

Embodiment 1455. The RNAi agent of claim 1454, wherein G^{A} is selected from H or OH and X is SH.

Embodiment 1456. The RNAi agent of claim 1402-1455, wherein at least one internucleoside linking group of the RNAi sense oligomeric compound is an internucleoside linking group of Formula XVII.

Embodiment 1457. The RNAi agent of claim 1456, wherein at least one of the five 5′-most internucleoside linking groups of the RNAi sense oligomeric compound is an internucleoside linking group of Formula XVII.

Embodiment 1458. The RNAi agent of claim 1456, wherein at least two of the five 5′-most internucleoside linking groups of the RNAi sense oligomeric compound are internucleoside linking groups of Formula XVII.

Embodiment 1459. The RNAi agent of claim 1456, wherein the two 5′-most internucleoside linking groups of the RNAi sense oligomeric compound are internucleoside linking groups of Formula XVII.

Embodiment 1460. The RNAi agent of any of claims 1456-1459, wherein at least one of the five 3′-most internucleoside linking groups of the RNAi sense oligomeric compound is an internucleoside linking group of Formula XVII.

Embodiment 1461. The RNAi agent of any of claims 1456-1459, wherein at least two of the five 3′-most internucleoside linking groups of RNAi sense oligomeric compound is an internucleoside linking group of Formula XVII.

Embodiment 1462. The RNAi agent of any of claims 1456-1459, wherein the two 3′-most internucleoside linking groups are internucleoside linking groups of Formula XVII.

Embodiment 1463. The RNAi agent of claim 1456, wherein the two 3′-most and the two 5′-most internucleoside linking groups of the RNAi sense oligonucleotide are internucleoside linking groups of Formula XVII, and the remaining internucleoside linking groups of the RNAi sense oligonucleotide are phosphodiester internucleoside linkages.

Embodiment 1464. The RNAi agent of any of claims 1456-1463, wherein for each internucleoside linking group of Formula XVII, R₁ is H and T is SO₂Me.

Embodiment 1465. The RNAi agent of any of claims 1456-1464, wherein the RNAi sense oligomeric compound consists of 21 linked nucleosides, and the internucleoside linkage motif is selected from: ooooooooooooooooooaa, aaooooooooooooooooaa, ooooooooooooooooooaa, or ssooooaoaaaooooooooo, wherein each “a” represents an internucleoside linkage of Formula XVII, each “s” represents a phosphorothioate internucleoside linkage, and each “o” represents a phosphodiester internucleoside linkage.

Embodiment 1466. The RNAi agent of claim 1465, wherein the internucleoside linkage motif of the RNAi sense oligomeric compound is selected from ooooooooooooooooooaa, aaooooooooooooooooaa, or ooooooooooooooooooaa, wherein each “a” represents an internucleoside linkage of Formula XVII, each “s” represents a phosphorothioate internucleoside linkage, and each “o” represents a phosphodiester internucleoside linkage.

Embodiment 1467. The RNAi agent of claim 1465 or 1466, wherein the sugar motif of the RNAi sense oligomeric compound is selected from: yyyyyyfyfffyyyyyyyyyy or fyfyfyfyfyfyfyfyfyfyf, wherein “y” represents a 2′-OMe sugar moiety and “f” represents a 2′-F sugar moiety.

Embodiment 1468. The RNAi agent of any of claims 1465-1467 wherein each “a” is a mesyl phosphoramidate linkage.

Embodiment 1469. The RNAi agent of any of claims 1456-1468, wherein at least one region of the RNAi sense oligomeric compound has structure A, B, C, D, E or P.

Embodiment 1470. The RNAi agent of claim 1469, wherein at least one region having structure A, B, C, D, or E is at the 3′ end of the RNAi sense oligomeric compound.

Embodiment 1471. The RNAi agent of claim 1469, wherein at least one region having structure A, B, C, D, or E is at the 5′ end of the RNAi sense oligomeric compound.

Embodiment 1472. The RNAi agent of any of claims 1456-1471, wherein at least one region of the RNAi sense oligomeric compound has the formula (N_{g1})_{L1}(N_{g2})_{L2}(N_{g3})_{L3}, wherein each N_{g} is a nucleoside and each L is an internucleoside linking group; wherein each of L₁, and L₂ is a phosphodiester internucleoside linking group, a phosphorothioate internucleoside linking group, or an internucleoside linking group of Formula XVII:

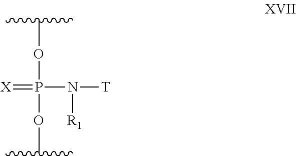

wherein L₃ is absent or is a phosphodiester internucleoside linking group, a phosphorothioate internucleoside linking group, or an internucleoside linking group of Formula XVII;
wherein at least one of L₁, L₂, and L₃ an internucleoside linking group of Formula XVII; and at least one of L₁, L₂, and L₃ is a phosphorothioate or a phosphodiester internucleoside linking group,
   wherein independently for each internucleoside linking group of the RNAi sense oligomeric compound having Formula XVII: X is selected from O or S;
   R₁ is selected from H, C₁-C₆ alkyl, and substituted C₁-C₆ alkyl; and
   T is selected from SO₂R₂, C(═O)R₃, and P(═O)R₄R₅, wherein:
   R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
   R₃ is selected from an aryl, a substituted aryl, CH₃, N(CH₃)₂, OCH₃ and a conjugate;
   R₄ is selected from OCH₃, OH, C₁-C₆ alkyl, substituted C₁-C₆ alkyl and a conjugate; and
   R₅ is selected from OCH₃, OH, C₁-C₆ alkyl, and substituted C₁-C₆ alkyl.
      Embodiment 1473. The RNAi agent of claim 1472, wherein at least one region having the formula (N_{g1})_{L1}(N_{g2})_{L2}(N_{g3})L₃ is at the 3′ end of the RNAi sense oligomeric compound.

Embodiment 1474. The RNAi agent of claim 1473, wherein L1 and L2 are internucleoside linking groups of Formula XVII, wherein R₁ is H and T is SO₂Me, and L3 is a phosphodiester internucleoside linkage.

Embodiment 1475. The RNAi agent of claim 1472, wherein at least one region having the formula (N_{g1})_{L1}(N_{g2})_{L2}(N_{g3})L₃ is at the 5′ end of the RNAi sense oligomeric compound.

Embodiment 1476. The RNAi agent of claim 1475, wherein L1 is a phosphodiester internucleoside linking group and L2 and L3 are each internucleoside linking groups of Formula XVII, wherein R₁ is H and T is SO₂Me.

Embodiment 1477. The RNAi agent of any of claims 1402-1476, wherein the RNAi sense oligomeric compound comprises a 3′ terminal group and/or a 5′ terminal group.

Embodiment 1478. The RNAi agent of any of claims 1402-1476, wherein the RNAi sense oligomeric compound comprises a conjugate group.

Embodiment 1479. The RNAi agent of claim 1478, wherein the conjugate group comprises a cell-targeting moiety.

Embodiment 1480. The RNAi agent of claim 1478, wherein the conjugate group comprises a carbohydrate or carbohydrate cluster.

Embodiment 1481. The RNAi agent of claim 1478, wherein the conjugate group comprises at least one GalNAc.

Embodiment 1482. The RNAi agent of claim 1478, wherein the conjugate group comprises a C₁₀-C₂₀ alkyl chain.

Embodiment 1483. The RNAi agent of claim 1478, wherein the conjugate group comprises C₁₆ alkyl.

Embodiment 1484. The RNAi agent of any of claims 1402-1483, wherein the double-stranded region of the oligonucleotide duplex is at least 15 nucleosides.

Embodiment 1485. The RNAi agent of any of claims 1402-1483, wherein the double-stranded region of the oligonucleotide duplex is at least 17 nucleosides.

Embodiment 1486. The RNAi agent of any of claims 1402-1483, wherein the double-stranded region of the oligonucleotide duplex is at least 19 nucleosides.

Embodiment 1487. The RNAi agent of any of claims 1402-1483, wherein the double-stranded region of the oligonucleotide duplex is exactly 19 nucleosides.

Embodiment 1488. The oligomeric compound of any of claims 1246-1383, wherein each nucleoside of the modified oligonucleotide is a modified nucleoside comprising a modified sugar moiety.

Embodiment 1489. The oligomeric compound of claim 1488, wherein each modified sugar moiety of the modified oligonucleotide is independently selected from a bicyclic sugar moiety and a 2′-substituted furanosyl sugar moiety.

Embodiment 1490. The oligomeric compound of claim 1488 or 1489, wherein each modified sugar moiety of the modified oligonucleotide comprises the same modification.

Embodiment 1491. The oligomeric compound of any of claims 1488-1490, wherein each modified sugar moiety of the modified oligonucleotide is selected from a 2′-OMe sugar moiety, a 2′-MOE sugar moiety, and a 2′-NMA sugar moiety.

Embodiment 1492. The oligomeric compound of claim 1488 or 1489, wherein the three 3′-most nucleosides of the modified oligonucleotide comprise a bicyclic sugar moiety, and the remaining nucleosides of the modified oligonucleotide comprise a 2′-substituted furanosyl sugar moiety.

Embodiment 1493. The oligomeric compound of claim 1488 or 1489, wherein the four 3′-most nucleosides of the modified oligonucleotide comprise a bicyclic sugar moiety, and the remaining nucleosides of the modified oligonucleotide comprise a 2′-substituted furanosyl sugar moiety.

Embodiment 1494. The oligomeric compound of claim 1488 or 1489, wherein the five 3′-most nucleosides of the modified oligonucleotide comprise a bicyclic sugar moiety, and the remaining nucleosides of the modified oligonucleotide comprise a 2′-substituted furanosyl sugar moiety.

Embodiment 1495. The oligomeric compound of claim 1488 or 1489, wherein the six 3′-most nucleosides of the modified oligonucleotide comprise a bicyclic sugar moiety, and the remaining nucleosides of the modified oligonucleotide comprise a 2′-substituted furanosyl sugar moiety.

Embodiment 1496. The oligomeric compound of any of claims 1488 or 1489, wherein each bicyclic sugar moiety of the modified oligonucleotide is selected from among cEt, LNA, and ENA.

Embodiment 1497. The oligomeric compound of claim 1496, wherein the bicyclic sugar moiety is cEt.

Embodiment 1498. The oligomeric compound of any of claims 1492-1497, wherein the 2′-substituted furanosyl sugar moiety is selected from 2′-OMe, 2′-MOE, and 2′-F.

Embodiment 1499. The oligomeric compound of any of claims 1488-1498, wherein at least one of the ten 5′-most linking groups of the modified oligonucleotide is an internucleoside linking group of Formula XVII.

Embodiment 1500. The oligomeric compound of claim 1499, wherein at least 2 of the ten 5′-most linking groups of the modified oligonucleotide are internucleoside linking groups of Formula XVII.

Embodiment 1501. The oligomeric compound of claim 1499, wherein at least 3 of the ten 5′-most linking groups of the modified oligonucleotide are internucleoside linking groups of Formula XVII.

Embodiment 1502. The oligomeric compound of claim 1499, wherein at least 4 of the ten 5′-most linking groups of the modified oligonucleotide are internucleoside linking groups of Formula XVII.

Embodiment 1503. The oligomeric compound of claim 1499, wherein at least 5 of the ten 5′-most linking groups of the modified oligonucleotide are internucleoside linking groups of Formula XVII.

Embodiment 1504. The oligomeric compound of claim 1499, wherein at least 6 of the ten 5′-most linking groups of the modified oligonucleotide are internucleoside linking groups of Formula XVII.

Embodiment 1505. The oligomeric compound of claim 1499, wherein the two 5′-most internucleoside linking groups are internucleoside linking groups of Formula XVII.

Embodiment 1506. The oligomeric compound of any of claims 1489-1505, wherein at least one of the ten 3′-most internucleoside linking groups of the modified oligonucleotide is an internucleoside linking group of Formula XVII.

Embodiment 1507. The oligomeric compound of claim 1506, wherein at least 2 of the ten 3′-most internucleoside linking groups of the modified oligonucleotide are internucleoside linking groups of Formula XVII.

Embodiment 1508. The oligomeric compound of claim 1506, wherein at least 3 of the ten 3′-most internucleoside linking groups are internucleoside linking groups of Formula XVII.

Embodiment 1509. The oligomeric compound of claim 1506, wherein at least 4 of the ten 3′-most internucleoside linking groups are internucleoside linking groups of Formula XVII.

Embodiment 1510. The oligomeric compound of claim 1506, wherein at least 5 of the ten 3′-most internucleoside linking groups are internucleoside linking groups of Formula XVII.

Embodiment 1511. The oligomeric compound of claim 1506, wherein at least 6 of the ten 3′-most internucleoside linking groups are internucleoside linking groups of Formula XVII.

Embodiment 1512. The oligomeric compound of claim 1506, wherein the two 3′-most internucleoside linking groups of the oligomeric compound are internucleoside linking groups of Formula XVII.

Embodiment 1513. The oligomeric compound of any of claims 1484-1494, wherein the modified oligonucleotide comprises at least one block of at least 3 consecutive internucleoside linking groups of Formula XVII.

Embodiment 1514. The oligomeric compound of any of claims 1484-1494, wherein the modified oligonucleotide comprises at least one block of at least 4 consecutive internucleoside linking groups of Formula XVII.

Embodiment 1515. The oligomeric compound of any of claims 1484-1494, wherein the modified oligonucleotide comprises at least one block of at least 5 consecutive internucleoside linking groups of Formula XVII.

Embodiment 1516. The oligomeric compound of any of claims 1484-1494, wherein the modified oligonucleotide comprises at least one block of at least 6 consecutive internucleoside linking groups of Formula XVII.

Embodiment 1517. The oligomeric compound of any of claims 1513-1516, wherein at least one block of consecutive internucleoside linking groups of Formula XVII is at the 5′ end of the modified oligonucleotide.

Embodiment 1518. The oligomeric compound of any of claims 1513-1516, wherein at least one block of consecutive internucleoside linking groups of Formula XVII is at the 3′ end of the modified oligonucleotide.

Embodiment 1519. The oligomeric compound of any of claims 1484-1518, wherein for each internucleoside linking group of Formula XVII of the modified oligonucleotide, R₁ is H and T is SO₂Me.

Embodiment 1520. The oligomeric compound of any of claims 1484-1494, wherein the internucleoside linkage motif of the modified oligonucleotide is selected from: aaaaaasssssssss, sssssaaaaaassss, or sssssssssaaaaaa, wherein each “a” represents an internucleoside linkage of Formula XVII, each “s” represents a phosphorothioate internucleoside linkage, and each “o” represents a phosphodiester internucleoside linkage.

Embodiment 1521. The oligomeric compound of claim 1520, wherein each “a” represents a mesyl phosphoramidate internucleoside linkage.

Embodiment 1522. The oligomeric compound of any of claims 1246-1383, wherein the modified oligonucleotide comprises a deoxy region consisting of 6-11 linked nucleosides wherein each nucleoside of the deoxy region is either a modified nucleoside or a stereo-standard DNA nucleoside and wherein at least 3 contiguous nucleosides of the deoxy region are stereo-standard DNA nucleosides and not more than three nucleosides of the deoxy region are modified nucleosides.

Embodiment 1523. The oligomeric compound of claim 1522, wherein at least 5 contiguous nucleosides of the deoxy region are stereo-standard DNA nucleosides.

Embodiment 1524. The oligomeric compound of claim 1522, wherein at least 6 contiguous nucleosides of the deoxy region are stereo-standard DNA nucleosides.

Embodiment 1525. The oligomeric compound of claim 1522, wherein at least 7 contiguous nucleosides of the deoxy region are stereo-standard DNA nucleosides.

Embodiment 1526. The oligomeric compound of claim 1522, wherein at least 8 contiguous nucleosides of the deoxy region are stereo-standard DNA nucleosides.

Embodiment 1527. The oligomeric compound of any of claims 1522-1526, wherein the deoxy region consists of 8-10 linked nucleosides.

Embodiment 1528. The oligomeric compound of any of claims 1522-1526, wherein the deoxy region consists of 9 linked nucleosides.

Embodiment 1529. The oligomeric compound of any of claims 1522-1526, wherein the deoxy region consists of 10 linked nucleosides.

Embodiment 1530. The oligomeric compound of any of claims 1522-1526, wherein the deoxy region consists of 11 linked nucleosides.

Embodiment 1531. The oligomeric compound of any of claims 1522-1526, wherein at least 6 nucleosides of the deoxy region are stereo-standard DNA nucleosides.

Embodiment 1532. The oligomeric compound of any of claims 1522-1526, wherein at least 7 nucleosides of the deoxy region are stereo-standard DNA nucleosides.

Embodiment 1533. The oligomeric compound of any of claims 1522-1526, wherein at least 8 nucleosides of the deoxy region are stereo-standard DNA nucleosides.

Embodiment 1534. The oligomeric compound of any of claims 1522-1526, wherein at least 9 nucleosides of the deoxy region are stereo-standard DNA nucleosides.

Embodiment 1535. The oligomeric compound of any of claims 1522-1534 wherein exactly two nucleosides of the deoxy region are modified nucleosides.

Embodiment 1536. The oligomeric compound of any of claims 1522-1535 wherein exactly one nucleoside of the deoxy region is a modified nucleoside.

Embodiment 1537. The oligomeric compound of any of claims 1522-1536 wherein at least one modified nucleoside of the deoxy region is a stereo-standard modified nucleoside or bicyclic nucleoside selected from a β-D-LNA nucleoside, an α-L-LNA nucleoside, an ENA nucleoside, a cEt nucleoside, a 2′-MOE nucleoside, a 2′-OMe nucleoside, a 2′-F nucleoside, and a 5′-alkyl nucleoside.

Embodiment 1538. The oligomeric compound of any of claims 1522-1537, wherein at least one modified nucleoside of the deoxy region is stereo-non-standard nucleoside.

Embodiment 1539. The oligomeric compound of claim 1538, wherein the at least one is stereo-non-standard nucleoside of the deoxy region is a stereo-non-standard DNA nucleoside.

Embodiment 1540. The oligomeric compound of claim 1539, wherein the stereo-non-standard DNA nucleoside is selected from a stereo-non-standard DNA nucleoside having: Formula I, Formula II, Formula III, Formula IV, Formula V, Formula VI, and Formula VII.

Embodiment 1541. The oligomeric compound of claim 1540, wherein the stereo-non-standard DNA nucleoside is selected from a stereo-non-standard DNA nucleoside having: Formula V and Formula II.

Embodiment 1542. The oligomeric compound of claim 1541, wherein at least one stereo-non-standard nucleoside of the deoxy region is a substituted stereo-non-standard nucleoside.

Embodiment 1543. The oligomeric compound of claim 1542, wherein at least one substituted stereo-non-standard nucleoside has a 2′-substituent selected from: 2′-MOE, 2′-OMe, 2′-F, or 2′-OH.

Embodiment 1544. The oligomeric compound of any of claims 1522-1543, wherein the 2^{nd} nucleoside from the 5′-end of the deoxy region is a modified nucleoside.

Embodiment 1545. The oligomeric compound of any of claims 1522-1543, wherein the 3^{rd} nucleoside from the 5′-end of the deoxy region is a modified nucleoside.

Embodiment 1546. The oligomeric compound of any of claims 1522-1543, wherein the 4^{th} nucleoside from the 5′-end of the deoxy region is a modified nucleoside.

Embodiment 1547. The oligomeric compound of any of claims 299-1546, wherein the modified nucleoside in the deoxy region is a 2′-OMe nucleoside.

Embodiment 1548. The oligomeric compound of any of claims 1522-1537, wherein each nucleoside of the deoxy region is a stereo-standard DNA nucleoside.

Embodiment 1549. The oligomeric compound of any of claims 1522-1548, wherein at least one internucleoside linking group within the deoxy region is an internucleoside linking group of Formula XVII.

Embodiment 1550. The oligomeric compound of any of claims 1522-1549, wherein the internucleoside linking group linking the 1^{t} and 2^{nd} nucleosides of the deoxy region as counted from the 5′-end of the deoxy region is an internucleoside linking group of Formula XVII.

Embodiment 1551. The oligomeric compound of any of claims 1522-1550, wherein the internucleoside linking group linking the 2^{nd} and 3^{rd} nucleosides of the deoxy region as counted from the 5′-end of the deoxy region is an internucleoside linking group of Formula XVII.

Embodiment 1552. The oligomeric compound of any of claims 1522-1551, wherein the internucleoside linking group linking the 3^{rd} and 4¹ nucleosides of the deoxy region as counted from the 5′-end of the deoxy region is an internucleoside linking group of Formula XVII.

Embodiment 1553. The oligomeric compound of any of claims 1522-1552, wherein the internucleoside linking group linking the 4^{th} and 5^{th} nucleosides of the deoxy region as counted from the 5′-end of the deoxy region is an internucleoside linking group of Formula XVII.

Embodiment 1554. The oligomeric compound of any of claims 1522-1553, wherein one internucleoside linking group in the deoxy region is a linking group of Formula XVII and the other internucleoside linking groups of the deoxy region are independently selected from phosphodiester and phosphorothioate internucleoside linking groups.

Embodiment 1555. The oligomeric compound of any of claims 1522-1555, wherein two internucleoside linking groups in the deoxy region are linking groups of Formula XVII and the other internucleoside linking groups of the deoxy region are independently selected from phosphodiester and phosphorothioate internucleoside linking groups.

Embodiment 1556. The oligomeric compound of any of claims 1522-1555, wherein three internucleoside linking groups in the deoxy region are linking groups linking groups of Formula XVII and the other internucleoside linking groups of the deoxy region are independently selected from phosphodiester and phosphorothioate internucleoside linking groups.

Embodiment 1557. The oligomeric compound of any of claims 1522-1555, wherein four internucleoside linking groups in the deoxy region are linking groups linking groups of Formula XVII and the other internucleoside linking groups of the deoxy region are each phosphodiester or phosphorothioate internucleoside linking groups.

Embodiment 1558. The oligomeric compound of any of claims 1554-1557, wherein the internucleoside linking groups of Formula XVII are linking the 1^{st} and 2^{nd}, 2^{nd} and 3^{rd}, 3^{rd} and 4^{th}, and/or the 4^{th} and 5^{th} nucleosides of the deoxy region, as counted from the 5′-end of the deoxy region.

Embodiment 1559. The oligomeric compound of any of claims 1522-1558, wherein the deoxy region comprises at least one region having structure A, B, C, D, E, or P.

Embodiment 1560. The oligomeric compound of claim 1559, wherein the region having structure A, B, C, D, or E is at the 3′ end of the deoxy region.

Embodiment 1561. The oligomeric compound of claim 1560, wherein the region having structure A, B, C, D, E, or P is at the 5′ end of the deoxy region.

Embodiment 1562. The oligomeric compound of any of claims 1522-1561, wherein the deoxy region comprises at least one region having the formula (N_{g1})_{L1}(N_{g2})_{L2}(N_{g3})_{L3}, wherein each N_{g} is a nucleoside and each L is an internucleoside linking group; wherein each of L₁, and L₂ is a phosphodiester internucleoside linking group, a phosphorothioate internucleoside linking group, or an internucleoside linking group of Formula XVII:

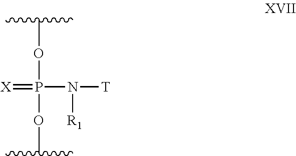

wherein L₃ is absent or is a phosphodiester internucleoside linking group, a phosphorothioate internucleoside linking group, or an internucleoside linking group of Formula XVII;
wherein at least one of L₁, L₂, and L₃ an internucleoside linking group of Formula XVII; and at least one of L₁, L₂, and L₃ is a phosphorothioate or a phosphodiester internucleoside linking group,
   wherein independently for each internucleoside linking group of the modified oligonucleotide having Formula XVII:
   X is selected from O or S;
   R₁ is selected from H, C₁-C₆ alkyl, and substituted C₁-C₆ alkyl; and
   T is selected from SO₂R₂, C(═O)R₃, and P(═O)R₄R₅, wherein:
   R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
   R₃ is selected from an aryl, a substituted aryl, CH₃, N(CH₃)₂, OCH₃ and a conjugate;
   R₄ is selected from OCH₃, OH, C₁-C₆ alkyl, substituted C₁-C₆ alkyl and a conjugate; and
      R₅ is selected from OCH₃, OH, C₁-C₆ alkyl, and substituted C₁-C₆ alkyl.

Embodiment 1563. The oligomeric compound of claim 1562, wherein the region having the formula (N_{g1})_{L1}(N_{g2})_{L2}(N_{g3})_{L3} is at the 3′ end of the deoxy region.

Embodiment 1564. The oligomeric compound of claim 1562, wherein the region having the formula (N_{g1})_{L1}(N_{g2})_{L2}(N_{g3})_{L3} is at the 5′ end of the deoxy region.

Embodiment 1565. The oligomeric compound of any of claims 1549-1564, wherein for each internucleoside linkage of Formula XVII, R₁ is H and T is SO₂Me.

Embodiment 1566. The oligomeric compound of any of claims 1522-1565, wherein the deoxy region is flanked on the 5′ side by a 5′-region consisting of 1-6 linked 5′-region nucleosides and on the 3′ side by a 3′-region consisting of 1-6 linked 3′-region nucleosides; wherein
the 3′-most nucleoside of the 5′-region comprises a modified sugar moiety; and
the 5′-most nucleoside of the 3′-region comprises a modified sugar moiety.

Embodiment 1567. The oligomeric compound of claim 1566, wherein the deoxy region consists of 7-11 linked nucleosides, and has the formula:
(N_{d1})_{L1}(N_{d2})_{L2}(N_{d3})_{L3}(N_{d4})_{L4}[(Nd)_{L5}]_{q};
wherein N_{d1}, N_{d2}, N_{d3}, N_{d4} are independently selected from among a stereo-standard DNA nucleoside, a stereo-non-standard DNA nucleoside, or a 2′-substituted nucleoside; with the proviso that no more than one of N_{d1}, N_{d}2, N_{d3}, or N_{d4} is a 2′-substituted nucleoside; each N_{d} is independently selected from among a stereo-standard DNA nucleoside and a stereo-non-standard DNA nucleoside;
q is from 3-8;
wherein each of L₁, L₂, L₃, L₄, and each L₅ is an internucleoside linkage;
wherein at least two of L₁, L₂, L₃, L₄ are internucleoside linkages of Formula XVII.

Embodiment 1568. The oligomeric compound of claim 1567, wherein one of N_{d1}, N_{d}2, N_{d3}, or N_{d4} is a 2′-substituted nucleoside.

Embodiment 1569. The oligomeric compound of claim 1568, wherein the 2′-substituted nucleoside is a 2′-OMe nucleoside.

Embodiment 1570. The oligomeric compound of claim 1569, wherein the 2′-OMe nucleoside is a stereo-standard 2′-OMe nucleoside.

Embodiment 1571. The oligomeric compound of any of claims 1566-1569, wherein the 2′-substituted nucleoside is N_{d2}.

Embodiment 1572. The oligomeric compound of claim 1567, wherein each of N_{d1}, N_{d}2, N_{d3}, N_{d4} and each N_{d} is a DNA nucleoside.

Embodiment 1573. The oligomeric compound of claim 1572, wherein each DNA nucleoside is a stereo-standard DNA nucleoside.

Embodiment 1574. The oligomeric compound of any of claims 1567-1573, wherein L₁ and L₂ are internucleoside linkages of Formula XVII.

Embodiment 1575. The oligomeric compound of any of claims 1567-1573, wherein L₂ and L₃ are internucleoside linkages of Formula XVII.

Embodiment 1576. The oligomeric compound of any of claims 1567-1573, wherein L₃ and L₄ are internucleoside linkages of Formula XVII.

Embodiment 1577. The oligomeric compound of any of claims 1567-1573, wherein L₁, L₂, and L₃ are internucleoside linkages of Formula XVII.

Embodiment 1578. The oligomeric compound of any of claims 1567-1573, wherein L₂, L₃, and L₄ are internucleoside linkages of Formula XVII.

Embodiment 1579. The oligomeric compound of any of claims 1567-1573, wherein L₁, L₂, L₃, and L₄ are internucleoside linkages of Formula XVII.

Embodiment 1580. The oligomeric compound of any of claims 1567-1579, wherein each L₅ is a phosphorothioate internucleoside linkage.

Embodiment 1581. The oligomeric compound of claims 1567-1580, wherein each internucleoside linkage that is not an internucleoside linkage of Formula XVII is a phosphorothioate internucleoside linkage.

Embodiment 1582. The oligomeric compound of any of claims 1567-1581, wherein for each internucleoside linkage of Formula XVII, R₁ is H and T is SO₂Me Embodiment 1583. The oligomeric compound of any of claims 1567-1582, wherein the 5′-region consists of 2-5 linked nucleosides.

Embodiment 1584. The oligomeric compound of claim 1583, wherein the 5′-region consists of 3 linked nucleosides.

Embodiment 1585. The oligomeric compound of claim 1583, wherein the 5′-region consists of 5 linked nucleosides.

Embodiment 1586. The oligomeric compound of any of claims 1566-1585 wherein each nucleoside of the 5′-region is a modified nucleoside.

Embodiment 1587. The oligomeric compound of any of claims 1566-1586, wherein each nucleoside of the 5′-region is a modified nucleoside comprising a modified sugar.

Embodiment 1588. The oligomeric compound of any of claims 1566-1587, wherein at least one nucleoside of the 5′-region comprises a 2′-substituted furanosyl sugar moiety.

Embodiment 1589. The oligomeric compound of any of claims 1566-1588, wherein each nucleoside of the 5′-region comprises a 2′-substituted furanosyl sugar moiety.

Embodiment 1590. The oligomeric compound of any of claims 1566-1589, wherein each 2′-substituted furanosyl sugar moiety of the 5′-region has a 2′-substituent selected from among 2′-MOE, 2′-OMe, and 2′-NMA.

Embodiment 1591. The oligomeric compound of any of claims 1566-1588 or 1590, wherein at least one nucleoside of the 5′-region comprises abicyclic furanosyl sugar moiety.

Embodiment 1592. The oligomeric compound of any of claims 1566-1588 or 1590-1591, wherein each nucleoside of the 5′-region comprises a bicyclic furanosyl sugar moiety.

Embodiment 1593. The oligomeric compound of claim 1591 or 1592, wherein each bicyclic sugar moiety of the 5′-region is selected from among cEt, LNA, and ENA.

Embodiment 1594. The oligomeric compound of claim 1593, wherein each bicyclic sugar moiety of the 5′-region is a cEt sugar moiety.

Embodiment 1595. The oligomeric compound of any of claims 1566-1594, wherein at least one nucleoside of the 5′ region is a stereo-standard DNA nucleoside.

Embodiment 1596. The oligomeric compound of any of claims 1566-1595, wherein at least one nucleoside of the 5′ region is a stereo-non-standard nucleoside.

Embodiment 1597. The oligomeric compound of any of claims 1566-1596, wherein each nucleobase of the 5′-region is independently selected from among thymine, uracil, guanine, cytosine, 5-methylcytosine, and adenine.

Embodiment 1598. The oligomeric compound of any of claims 1566-1597, wherein the 3′-region consists of 2-5 linked nucleosides.

Embodiment 1599. The oligomeric compound of claim 1598, wherein the 3′-region consists of 3 linked nucleosides.

Embodiment 1600. The oligomeric compound of claim 1598, wherein the 3′-region consists of 5 linked nucleosides.

Embodiment 1601. The oligomeric compound of any of claims 1566-1600, wherein each nucleoside of the 3′-region is a modified nucleoside.

Embodiment 1602. The oligomeric compound of any of claims 1566-1601, wherein each nucleoside of the 3′-region is a modified nucleoside comprising a modified sugar.

Embodiment 1603. The oligomeric compound of any of claims 1566-1602, wherein at least one nucleoside of the 3′-region comprises a 2′-substituted furanosyl sugar moiety.

Embodiment 1604. The oligomeric compound of any of claims 1566-1603, wherein each nucleoside of the 3′-region comprises a 2′-substituted furanosyl sugar moiety.

Embodiment 1605. The oligomeric compound of any of claims 1566-1604, wherein each 2′-substituted furanosyl sugar moiety of the 3′-region has a 2′-substituent selected from among 2′-MOE, 2′-OMe, and 2′-NMA.

Embodiment 1606. The oligomeric compound of any of claims 1566-1603 or 1605, wherein at least one nucleoside of the 3′-region comprises abicyclic furanosyl sugar moiety.

Embodiment 1607. The oligomeric compound of any of claims 1566-1603 or 1606, wherein each nucleoside of the 3′-region comprises a bicyclic furanosyl sugar moiety.

Embodiment 1608. The oligomeric compound of claim 1606 or 1607, wherein each bicyclic sugar moiety of the 3′-region is selected from among cEt, LNA, and ENA.

Embodiment 1609. The oligomeric compound of claim 1608, wherein each bicyclic sugar moiety of the 3′-region is a cEt sugar moiety.

Embodiment 1610. The oligomeric compound of any of claims 1566-1609, wherein at least one nucleoside of the 3′ region is a stereo-standard DNA nucleoside.

Embodiment 1611. The oligomeric compound of any of claims 1566-1610, wherein at least one nucleoside of the 3′ region is a stereo-non-standard nucleoside.

Embodiment 1612. The oligomeric compound of any of claims 1566-1611, wherein each nucleobase of the 3′-region is independently selected from among thymine, uracil, guanine, cytosine, 5-methylcytosine, and adenine.

Embodiment 1613. The oligomeric compound of any of claims 1566-1612 wherein the oligomeric compound is a gapmer.

Embodiment 1614. The oligomeric compound of any of claims 1566-1613, wherein the modified oligonucleotide has a sugar motif selected from kkkddddddddddkkk and kkkdyddddddddkkk, wherein each “k” represents a cEt sugar moiety, “y” represents a 2′-OMe sugar moiety, and each “d” represents a β-D-2′-deoxyribosyl sugar moiety.

Embodiment 1615. The oligomeric compound of any of claims 1566-1614, wherein the modified oligonucleotide has an internucleoside linkage motif selected from: ssssssssssssssa, sssssssssssssas, ssssssssssssass, sssssssssssasss, ssssssssssassss, sssssssssasssss, ssssssssassssss, sssssssasssssss, ssssssassssssss, sssssasssssssss, ssssassssssssss, sssasssssssssss, ssassssssssssss, sasssssssssssss, assssssssssssss, sssssssssssssaa, ssssssssssssaas, sssssssssssaass, ssssssssssaasss, sssssssssaassss, ssssssssaasssss, sssssssaassssss, ssssssaasssssss, sssssaassssssss, ssssaasssssssss, sssaassssssssss, ssaasssssssssss, saassssssssssss, aasssssssssssss, aaaaaaaaaaaaaaa, ssaaaaaaaaaaass, ssaaaaaaaaaasss, sssaaaaaaaaasss, aassssssssssaaa, sssaaasssssssss, ssssaaassssssss, sssaaaassssssss, ssaaassssssssss, ssaaaasssssssss, ssaaaaassssssss, ssaaaaaasssssss, ssaaaaaaassssss, ssaaaaaaaasssss, ssaaaaaaaaassss, ssssssssssaaass, sssssssssaaaass, ssssssssaaaaass, sssssssaaaaaass, ssssssaaaaaaass, sssssaaaaaaaass, ssssaaaaaaaaass, sssaaaaaaaaaass, ssasasasasasass, sssasasasasasss, ooossssssssssoo, soossssssssssos, aoosssssssssooa, aoasssssssssaoa, aoaaaassssssaoa, aoossssssssssoa, ooasssssssssaoo, aoosaasssssssoa, aossaasssssssoa, aooaaaasssssaoa, aoossssssaaaaoa, sssssaaasssssss, ssssssaaassssss, sssssssaaasssss, ssssssssaaassss, sssssssssaaasss, ssssaaaasssssss, sssssaaaassssss, ssssssaaaasssss, sssssssaaaassss, ssssssssaaaasss, sssaassssssaass, sssaasssssaasss, sssaassssaassss, sssaasssaasssss, sssaassaassssss, sssaasaasssssss, ssaasssssssaass, ssssaasssssaass, sssssaassssaass, ssssssaasssaass, sssssssaassaass, ssssssssaasaass, ssssaasssaassss, sssssaasaasssss, sssssssssssssss, aaassssssssssss, aaassssssssssaa,aaaaaasssssssss, aoooosaasssssssooaa, aoooossssssssssooaa, sooooaassssssssooss, soooosaasssssssooss, soooossaassssssooss, soooossssssaassooss, sssaaaaasssssss, sssssaaaaaassss, or sssssssssaaaaaa, wherein each “a” represents an internucleoside linkage of Formula XVII, each “s” represents a phosphorothioate internucleoside linkage, and each “o” represents a phosphodiester internucleoside linkage.

Embodiment 1616. The oligomeric compound of claim 1615, wherein the modified oligonucleotide has an internucleoside linkage motif selected from: sssaaaassssssss, sssaaasssssssss, ssssaaassssssss, ssssaasssssaass, sssaassssssssss, ssssaasssssssss, sssssaassssssss, or sssssssssaassss, wherein each “a” represents an internucleoside linkage of Formula XVII, each “s” represents a phosphorothioate internucleoside linkage, and each “o” represents a phosphodiester internucleoside linkage.

Embodiment 1617. The oligomeric compound of claim 1615 or 1616, wherein each “a” represents a mesyl phosphoramidate internucleoside linkage.

Embodiment 1618. The oligomeric compound of any of claims 1246-1383, wherein the modified oligonucleotide is a CRISPR compound.

Embodiment 1619. The oligomeric compound of claim 1618, wherein the CRISPR compound consists of 20-50 or 29-32 linked nucleosides.

Embodiment 1620. The oligomeric compound of any of claims 1335-1619, wherein each X is O.

Embodiment 1621. The oligomeric compound of any of claims 1335-1619, wherein each X is S.

Embodiment 1622. The oligomeric compound of any of claims 1335-1621, wherein at least one R₁ is H.

Embodiment 1623. The oligomeric compound of any of claims 1335-1621, wherein at least one R₁ is a C₁-C₆ alkyl.

Embodiment 1624. The oligomeric compound of claim 1623, wherein the at least one R₁ is methyl.

Embodiment 1625. The oligomeric compound of any of claims 1335-1621, at least one R₁ is a substituted C₁-C₆ alkyl.

Embodiment 1626. The oligomeric compound of any of claims 1335-1625, wherein at least one T comprises a conjugate group.

Embodiment 1627. The oligomeric compound of claim 1626, wherein the conjugate group comprises a cell-targeting moiety.

Embodiment 1628. The oligomeric compound of claim 1626, wherein the conjugate group comprises a carbohydrate or carbohydrate cluster.

Embodiment 1629. The oligomeric compound of any of claims 1626-1627, wherein the conjugate group comprises at least one GalNAc.

Embodiment 1630. The oligomeric compound of claim 1626, wherein the conjugate group comprises a C₁₀-C₂₀ alkyl chain.

Embodiment 1631. The oligomeric compound of claim 1630, wherein the conjugate group comprises C₁₆ alkyl.

Embodiment 1632. The oligomeric compound of any of claims 1335-1631, wherein at least one T does not comprise a conjugate group.

Embodiment 1633. The oligomeric compound of any of claims 1335-1625, wherein each T does not comprise a conjugate group.

Embodiment 1634. The oligomeric compound of any of claims 1335-1633, wherein at least one T is SO₂R₂.

Embodiment 1635. The oligomeric compound of claim 1634, wherein R₂ is an aryl.

Embodiment 1636. The oligomeric compound of claim 1634, wherein R₂ is a substituted aryl.

Embodiment 1637. The oligomeric compound of claim 1634, wherein R₂ is a heterocycle.

Embodiment 1638. The oligomeric compound of claim 1634, wherein R₂ is a substituted heterocycle.

Embodiment 1639. The oligomeric compound of claim 1634, wherein R₂ is an aromatic heterocycle.

Embodiment 1640. The oligomeric compound of claim 1634, wherein R₂ is a substituted aromatic heterocycle.

Embodiment 1641. The oligomeric compound of claim 1634, wherein R₂ is a diazole.

Embodiment 1642. The oligomeric compound of claim 1634, wherein R₂ is a substituted diazole.

Embodiment 1643. The oligomeric compound of claim 1634, wherein R₂ is an amine.

Embodiment 1644. The oligomeric compound of claim 1634, wherein R₂ is a substituted amine.

Embodiment 1645. The oligomeric compound of claim 1634, wherein R₂ is a C₁-C₆ alkoxy, C₁-C₆ alkenyl, or C₁-C₆ alkynyl.

Embodiment 1646. The oligomeric compound of claim 1634, wherein R₂ is C₁-C₂₀, C₁-C₆, C₂-C₂₀, C₂-C₆, or C₁₀-C₂₀ alkyl.

Embodiment 1647. The oligomeric compound of claim 1634, wherein R₂ is substituted C₁-C₂₀, C₁-C₆, C₂-C₂₀, C₂-C₆, or C₁₀-C₂₀ alkyl.

Embodiment 1648. The oligomeric compound of claim 1634, wherein R₂ comprises a carbohydrate or carbohydrate cluster.

Embodiment 1649. The oligomeric compound of claim 1634, wherein R₂ comprises at least one GalNAc.

Embodiment 1650. The oligomeric compound of claim 1634, wherein T is:

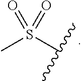 [SMILES: *S(C)(=O)=O]

Embodiment 1651. The oligomeric compound of claim 1634, wherein T is:

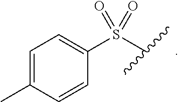 [SMILES: *S(=O)(=O)c1ccc(C)cc1]

Embodiment 1652. The oligomeric compound of claim 1634, wherein T is:

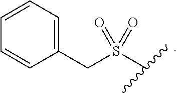 [SMILES: *S(=O)(=O)Cc1ccccc1]

Embodiment 1653. The oligomeric compound of claim 1634, wherein T is:

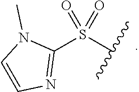 [SMILES: *S(=O)(=O)c1nccn1C]

Embodiment 1654. The oligomeric compound of claim 1634, wherein T is:

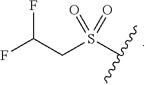 [SMILES: *S(=O)(=O)CC(F)F]

Embodiment 1655. The oligomeric compound of claim 1634, wherein T is:

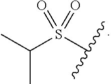 [SMILES: *S(=O)(=O)C(C)C]

Embodiment 1656. The oligomeric compound of claim 1634, wherein T is:

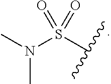 [SMILES: *S(=O)(=O)N(C)C]

Embodiment 1657. The oligomeric compound of claim 1634, wherein T is:

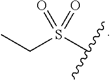 [SMILES: *S(=O)(=O)CC]

Embodiment 1658. The oligomeric compound of claim 1634, wherein T is:

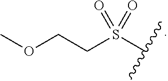 [SMILES: *S(=O)(=O)CCOC]

Embodiment 1659. The oligomeric compound of claim 1634, wherein T is:

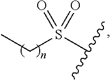

wherein n is from 2 to 20.

Embodiment 1660. The oligomeric compound of claim 1659, wherein n is 15.

Embodiment 1661. The oligomeric compound of any of claims 1335-1660, wherein at least one T is C(═O)R₃.

Embodiment 1662. The oligomeric compound of claim 1661, wherein R₃ is an aryl.

Embodiment 1663. The oligomeric compound of claim 1661, wherein R₃ is a substituted aryl.

Embodiment 1664. The oligomeric compound of claim 1661, wherein R₃ is CH₃.

Embodiment 1665. The oligomeric compound of claim 1661, wherein R₃ is N(CH₃)2.

Embodiment 1666. The oligomeric compound of claim 1661, wherein R₃ is OCH₃.

Embodiment 1667. The oligomeric compound of claim 1661, wherein R₃ is a C₁-C₆ alkoxy.

Embodiment 1668. The oligomeric compound of claim 1661, wherein R₃ is C₁-C₂₀, C₁-C₆, C₂-C₂₀, C₂-C₆, or C₁₀-C₂₀ alkyl.

Embodiment 1669. The oligomeric compound of claim 1661, wherein R₃ is substituted C₁-C₂₀, C₁-C₆, C₂-C₂₀, C₂-C₆, or C₁₀-C₂₀ alkyl.

Embodiment 1670. The oligomeric compound of claim 1661, wherein R₃ comprises a carbohydrate or carbohydrate cluster.

Embodiment 1671. The oligomeric compound of claim 1661, wherein R₂₃ comprises at least one GalNAc.

Embodiment 1672. The oligomeric compound of claim 1661, wherein T is:

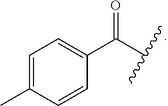 [SMILES: *C(=O)c1ccc(C)cc1]

Embodiment 1673. The oligomeric compound of claim 1661, wherein T is:

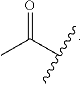 [SMILES: *C(C)=O]

Embodiment 1674. The oligomeric compound of claim 1661, wherein T is:

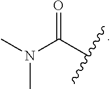 [SMILES: *C(=O)N(C)C]

Embodiment 1675. The oligomeric compound of claim 1661, wherein T is:

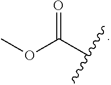 [SMILES: *C(=O)OC]

Embodiment 1676. The oligomeric compound of claim 1661, wherein T is:

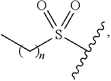

wherein n is from 2 to 20.

Embodiment 1677. The oligomeric compound of claim 1676, wherein n is 15.

Embodiment 1678. The oligomeric compound of any of claims 1335-1677, wherein at least one T is P(═O)R₄R₅.

Embodiment 1679. The oligomeric compound of claim 1678, wherein R₄ is OCH₃.

Embodiment 1680. The oligomeric compound of claim 1678, wherein R₄ is OH

Embodiment 1681. The oligomeric compound of claim 1678, wherein R₄ is C₁-C₆ alkyl.

Embodiment 1682. The oligomeric compound of claim 1678, wherein R₄ is substituted C₁-C₆ alkyl.

Embodiment 1683. The oligomeric compound of claim 1678, wherein R₄ is C₁-C₂₀, C₁-C₆, C₂-C₂₀, C₂-C₆, or C₁₀-C₂₀ alkyl.

Embodiment 1684. The oligomeric compound of claim 1678, wherein R₄ is substituted C₁-C₂₀, C₁-C₆, C₂-C₂₀, C₂-C₆, or C₁₀-C₂₀ alkyl.

Embodiment 1685. The oligomeric compound of claim 1678, wherein R₄ comprises a carbohydrate or carbohydrate cluster.

Embodiment 1686. The oligomeric compound of claim 1678, wherein R₄ comprises at least one GalNAc.

Embodiment 1687. The oligomeric compound of any of claims 1678-1686, wherein R₅ is OCH₃.

Embodiment 1688. The oligomeric compound of any of claims 1678-1686, wherein R₅ is OH.

Embodiment 1689. The oligomeric compound of any of claims 1678-1686, wherein R₅ is C₁-C₆ alkyl.

Embodiment 1690. The oligomeric compound of any of claims 1678-1686, wherein R₅ is substituted C₁-C₆ alkyl.

Embodiment 1691. The oligomeric compound of claim 1678, wherein T is:

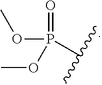 [SMILES: *P(=O)(OC)OC]

Embodiment 1692. The oligomeric compound of claim 1678, wherein T is:

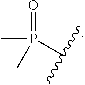 [SMILES: *P(C)(C)=O]

Embodiment 1693. The oligomeric compound of claim 1678, wherein T is:

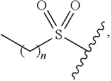

wherein n is from 2 to 20.

Embodiment 1694. The oligomeric compound of claim 1693, wherein n is 15.

Embodiment 1695. An antisense agent comprising a modified oligonucleotide consisting of 12-50 linked nucleosides linked through internucleoside linking groups, wherein at least one internucleoside linking group is a phosphodiester or a phosphorothioate internucleoside linking group, and wherein at least one of the internucleoside linking groups has Formula XX:

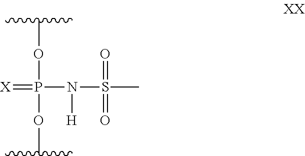

wherein independently for each internucleoside linking group of the modified oligonucleotide having Formula XX, X is selected from O or S.

Embodiment 1696. An antisense agent comprising a modified oligonucleotide, wherein the 5′-terminus of the modified oligonucleotide has Structure F:

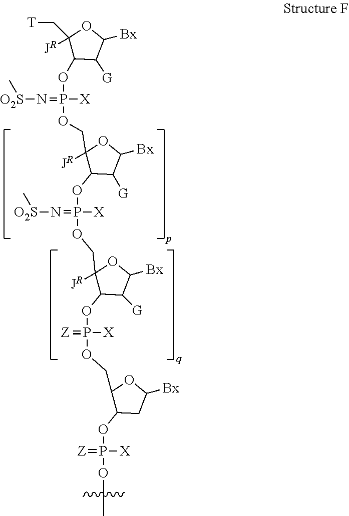

wherein:
p is from 0 to 6;
q is from 0 to 6;
T is OH or a conjugate group;
each Bx is an independently selected heterocyclic base moiety;
each X is independently selected from OH or SH;
each Z is independently selected from O, S, or NSO₂Me;
For each J^{R} and G of the same furanosyl sugar moiety, either J^{R} and G form a J^{R} to G bridge, or J^{R} is H and G is selected from OH, halogen or O—[C(R₆)(R₇)]ₙ—[(C═O)ₘ—X^{G}]ⱼ—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
   R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X2)N(J₁)(J₂) and C(=Q₂)N(J₁)(J₂);
Q₂ is O, S or NJ₃;
each J₁, J₂ and J₃ is, independently, H or C₁-C₆ alkyl.

Embodiment 1697. An antisense agent comprising a modified oligonucleotide, wherein the 3′-terminus of the modified oligonucleotide has Structure G: T G

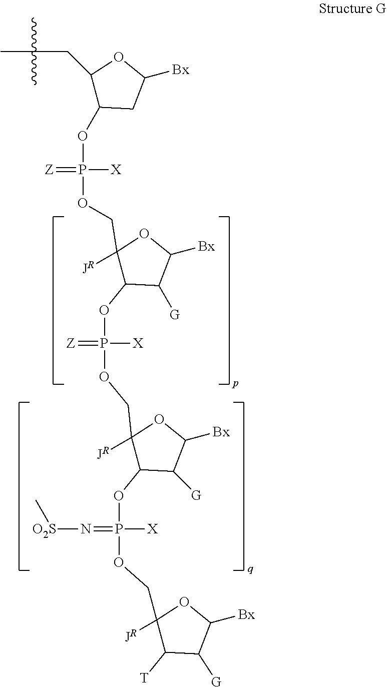

wherein:
p is from 0 to 6;
q is from 1 to 6;
T is OH or a conjugate group;
each Bx is an independently selected heterocyclic base moiety;
each X is independently selected from OH or SH;
each Z is independently selected from O, S, or NSO₂Me;
For each J^{R} and G of the same furanosyl sugar moiety, either J^{R} and G form a J^{R} to G bridge, or J^{R} is H and G is selected from OH, halogen or O—[C(R₆)(R₇)]ₙ—[(C═O)ₘ—X^{G}]ⱼ—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

Embodiment 1698. The antisense agent of claim 1696 or 1697, wherein the sum of p+q is selected from 2, 3, 4, or 5.

Embodiment 1699. An antisense agent comprising a modified oligonucleotide, wherein the 5′-terminus of the modified oligonucleotide has Structure H:

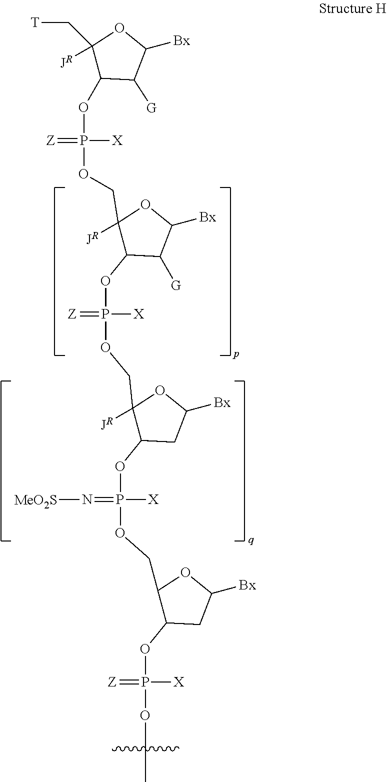

wherein:
p is from 0 to 5;
q is from 1 to 4;
T is OH or a conjugate group;
each Bx is an independently selected heterocyclic base moiety;
each X is independently selected from OH or SH;
each Z is independently selected from O, S, or NSO₂Me;
For each J^{R} and G of the same furanosyl sugar moiety, either J^{R} and G form a J^{R} to G bridge, or J^{R} is H and G is selected from OH, halogen or O—[C(R₆)(R₇)]ₙ[(C═O)ₘ—X^{G}]ⱼ—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

Embodiment 1700. An antisense agent comprising a modified oligonucleotide, wherein the 5′-terminus of the modified oligonucleotide has Structure I:

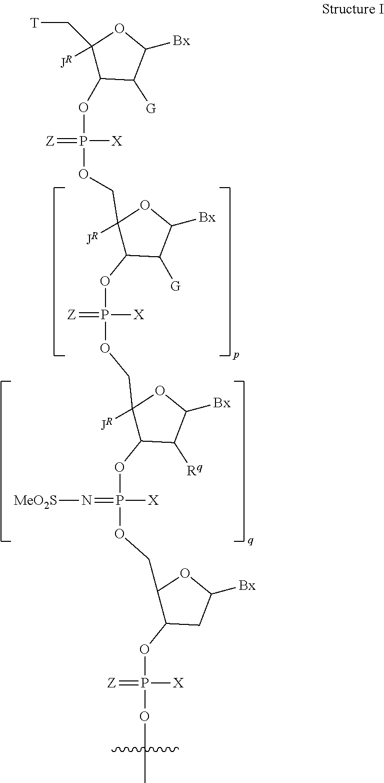

wherein:
p is from 0 to 5;
q is from 1 to 4;
T is OH or a conjugate group;
each Bx is an independently selected heterocyclic base moiety;
each X is independently selected from OH or SH;
each Z is independently selected from O, S, or NSO₂Me;
each R^{q} is H or exactly one R^{q} is OMe and the other R^{q} are H;
For each J^{R} and G of the same furanosyl sugar moiety, either J^{R} and G form a J^{R} to G bridge, or J^{R} is H and G is selected from OH, halogen or O—[C(R₆)(R₇)]ₙ—[(C═O)ₘ—X^{G}]ⱼ—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

Embodiment 1701. The antisense agent of claim 1700, wherein exactly one R^{q} is —OMe.

Embodiment 1702. The antisense agent of any of claims 1699-1701, wherein the sum of p+q is 2, 3, or 4.

Embodiment 1703. An antisense agent comprising a modified oligonucleotide, wherein the 3′-terminus of the modified oligonucleotide has Structure J:

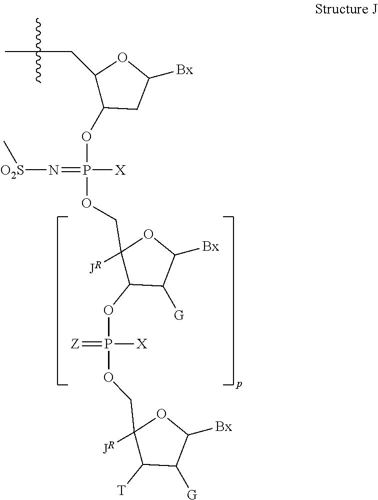

wherein:
p is from 0 to 6;
T is OH or a conjugate group;
each Bx is an independently selected heterocyclic base moiety;
each X is independently selected from OH or SH;
each Z is independently selected from O, S, or NSO₂Me;
For each J^{R} and G of the same furanosyl sugar moiety, either J^{R} and G form a J^{R} to G bridge, or J^{R} is H and G is selected from OH, halogen or O—[C(R₆)(R₇)]ₙ—[(C═O)ₘ—X^{G}]ⱼ—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

Embodiment 1704. The antisense agent of claim 1703, wherein p is 2, 3, or 4.

Embodiment 1705. The antisense agent of any of claims 1696-1704, wherein each J^{R} is H and each G is OCH₂CH₂OCH₃.

Embodiment 1706. The antisense agent of any of claims 1696-1704, wherein each J^{R} is H and each G is OCH₃.

Embodiment 1707. The antisense agent of any of claims 1696-1704, wherein each J^{R} and G form a J^{R} to G bridge.

Embodiment 1708. The antisense agent of claim 1707, wherein the J^{R} to G bridge has the formula —CH(CH₃)—O—.

Embodiment 1709. The antisense agent of claim 1695, wherein the antisense agent is an RNAi agent.

Embodiment 1710. The RNAi agent of claim 1709, wherein the RNAi agent is a single-stranded RNAi agent comprising an RNAi antisense modified oligonucleotide.

Embodiment 1711. The RNAi agent of claim 1709, wherein the RNAi agent is an oligonucleotide duplex comprising an RNAi antisense modified oligonucleotide and an RNAi sense modified oligonucleotide.

Embodiment 1712. The RNAi agent of any of claims 1710-1711, wherein the 5′-terminus of the RNAi antisense oligonucleotide has structure K:

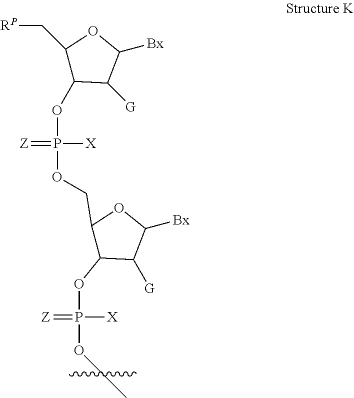

wherein:
R^{P} is a phosphate, stabilized phosphate group, or a mesyl phosphoramidate;
each Bx is an independently selected heterocyclic base moiety;
each X is independently selected from OH or SH;
each Z is selected from O, S, or NSO₂Me;
at least one Z is NSO₂Me;
each G is independently selected from OH, halogen or O—[C(R₆)(R₇)]ₙ—[(C═O)ₘ—X^{G}]ⱼ—R₈; each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

Embodiment 1713. The RNAi agent of claim 1712, wherein the stabilized phosphate group is 5′-vinyl phosphonate or 5′-cyclopropyl phosphonate.

Embodiment 1714. The RNAi agent of claim 1712, wherein the stabilized phosphate group is a mesyl phosphoramidate.

Embodiment 1715. The RNAi agent of any of claims 1712-1714, wherein each G within structure K is independently selected from F or OMe.

Embodiment 1716. The RNAi agent of any of claims 1712-1715, wherein the 3′-terminus of the RNAi antisense oligonucleotide has structure L:

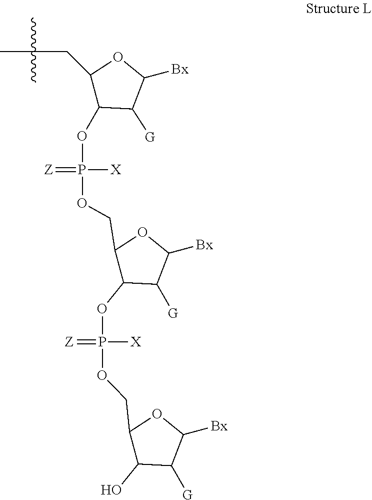

wherein:
   each Bx is an independently selected heterocyclic base moiety;
   each X is independently selected from OH or SH;
   each Z is selected from O, S, or NSO₂Me;
   at least one Z is NSO₂Me;
   each G is independently selected from OH, halogen or O—[C(R₆)(R₇)]ₙ—[(C═O)ₘ—X^{G}],R₈;
   each R₆ and R₇ is, independently, H, halogen, C₁-C₆ alkyl or substituted C₁-C₆ alkyl;
   each X^{G} is O, S or N(E₁);
      R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
   E₁, E₂ and E₃ are each, independently, H, C₁-C₆ alkyl or substituted C₁-C₆ alkyl;
   n is from 1 to 6;
   m is 0 or 1;
   j is 0 or 1;
   each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
   Q₂ is O, S or NJ₃;
   each J₁, J₂ and J₃ is, independently, H or C₁-C₆ alkyl.

Embodiment 1717. The RNAi agent of claim 1716, wherein each G within Structure L of the RNAi antisense oligonucleotide is independently selected from F or OMe.

Embodiment 1718. The RNAi agent of any of claims 1710-1717, wherein at least one region of the RNAi antisense oligonucleotide has structure M:

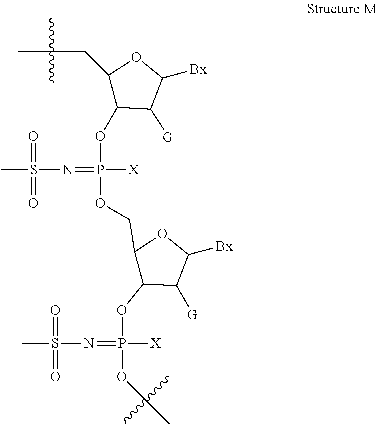

wherein:
   each Bx is an independently selected heterocyclic base moiety;
   each X is independently selected from OH or SH;
   each G is independently selected from OH, halogen or O—[C(R₆)(R₇)]ₙ[(C═O)ₘ—X^{G}]ⱼ—R₈;
   each R₆ and R₇ is, independently, H, halogen, C₁-C₆ alkyl or substituted C₁-C₆ alkyl;
   each X^{G} is O, S or N(E₁);
      R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
   E₁, E₂ and E₃ are each, independently, H, C₁-C₆ alkyl or substituted C₁-C₆ alkyl;
   n is from 1 to 6;
   m is 0 or 1;
   j is 0 or 1;
      each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
   Q₂ is O, S or NJ₃;
   each J₁, J₂ and J₃ is, independently, H or C₁-C₆ alkyl.

Embodiment 1719. The RNAi agent of claim 1718, wherein each G of Structure M within the RNAi antisense oligonucleotide is selected from F or OMe.

Embodiment 1720. The RNAi agent of claim 1719, wherein one G is F and the other G is OMe.

Embodiment 1721. The RNAi agent of any of claims 1710-1711 or 1716-1720, wherein the 5′-terminus of the RNAi antisense oligonucleotide has structure N:

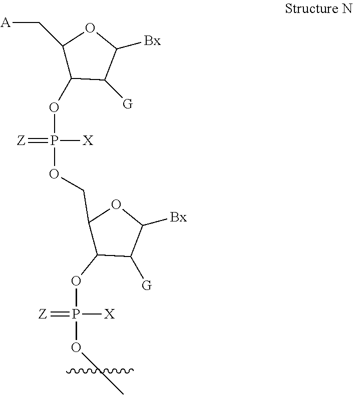

wherein:
A is selected from or

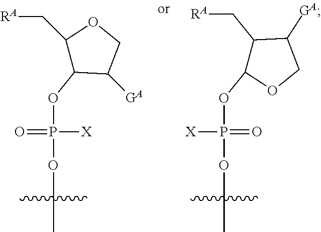

R^{A} is OH, OP(═O)OH, OP(═O)SH, a stabilized phosphate group, or a mesyl phosphoramidate;
G^{A} is H, OH, OMe, MOE, or a halogen;
each Bx is an independently selected heterocyclic base moiety;
each X is independently selected from OH or SH;
each G is independently selected from OH, halogen or O—[C(R₆)(R₇)]ₙ[(C═O)ₘ—X^{G}]ⱼ—R₈; each R₆ and R₇ is, independently, H, halogen, C₁-C₆ alkyl or substituted C₁-C₆ alkyl;
each X^{G} is O, S or N(E₁);
   R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

Embodiment 1722. The RNAi agent of claim 1722, wherein each G within structure N of the RNAi antisense oligonucleotide is selected from F or OMe.

Embodiment 1723. The RNAi agent of any of claims 1710-1714 or 1718-1722, wherein the 3′-terminus of the RNAi antisense oligonucleotide has structure O:

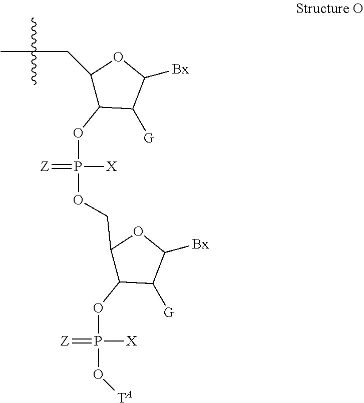

wherein:

T^{A} is selected from

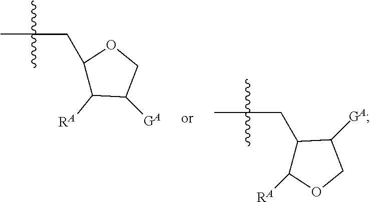

R^{A} is OH, OP(═O)OH, OP(═O)SH, or a stabilized phosphate group;
G^{A} is H, OH, OMe, MOE, or a halogen;
each Bx is an independently selected heterocyclic base moiety;
each X is independently selected from OH or SH;
each Z is selected from O, S, or NSO₂Me;
at least one Z is NSO₂Me;
each G is independently selected from OH, halogen or O—[C(R₆)(R₇)]ₙ—[(C═O)ₘ—X^{G}]ⱼ—R₈;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

Embodiment 1724. The RNAi agent of claim 1723, wherein each G within structure O of the RNAi antisense oligonucleotide is selected from F or OMe.

Embodiment 1725. The RNAi agent of claim 1711, wherein the 5′-terminus of the RNAi sense oligonucleotide has structure K:

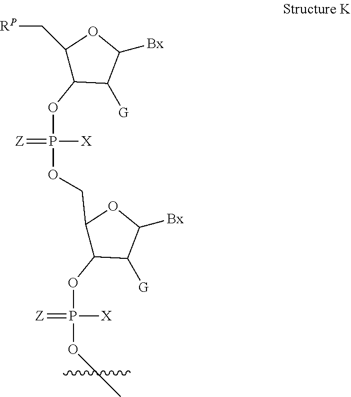

wherein:
R^{P} is a phosphate or stabilized phosphate group;
each Bx is an independently selected heterocyclic base moiety;
each X is independently selected from OH or SH;
each Z is selected from O, S, or NSO₂Me;
at least one Z is NSO₂Me;
each G is independently selected from OH, halogen or O—[C(R₆)(R₇)]ₙ-[(C═O)ₘ—X^{G}]ⱼ—R₈;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

Embodiment 1726. The RNAi agent of claim 1725, wherein the stabilized phosphate group is 5′-vinyl phosphonate,5′-cyclopropyl phosphonate, or 5′-mesyl phosphoramidate.

Embodiment 1727. The RNAi agent of claim 1725 or 1726, wherein each G within structure K is independently selected from F or OMe.

Embodiment 1728. The RNAi agent of any of claims 1711 or 1725-1727, wherein the 3′-terminus of the RNAi sense oligonucleotide has structure L:

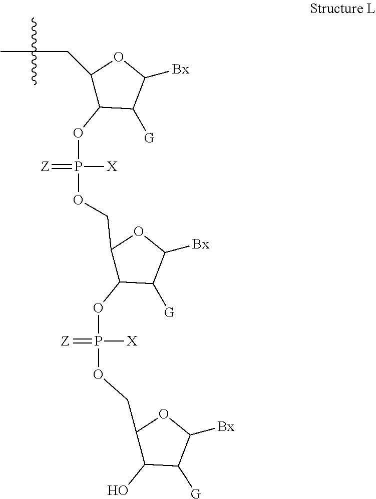

wherein:
   each Bx is an independently selected heterocyclic base moiety;
   each X is independently selected from OH or SH;
   each Z is selected from O, S, or NSO₂Me;
   at least one Z is NSO₂Me;
   each G is independently selected from OH, halogen or O—[C(R₆)(R₇)]ₙ[(C═O)ₘ—X^{G}]ⱼ—R₈;
   each R₆ and R₇ is, independently, H, halogen, C₁-C₆ alkyl or substituted C₁-C₆ alkyl;
   each X^{G} is O, S or N(E₁);
      R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
   E₁, E₂ and E₃ are each, independently, H, C₁-C₆ alkyl or substituted C₁-C₆ alkyl;
   n is from 1 to 6;
   m is 0 or 1;
   j is 0 or 1;
   each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
   Q₂ is O, S or NJ₃;
   each J₁, J₂ and J₃ is, independently, H or C₁-C₆ alkyl.

Embodiment 1729. The RNAi agent of claim 1728, wherein each G within Structure L of the RNAi sense oligonucleotide is independently selected from F or OMe.

Embodiment 1730. The RNAi agent of any of claims 1711 or 1725-1729 wherein at least one region of the RNAi sense oligonucleotide has structure M:

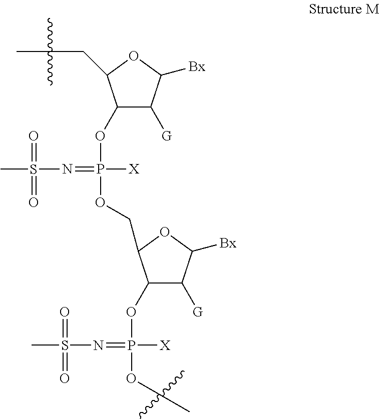

wherein:
   each Bx is an independently selected heterocyclic base moiety;
   each X is independently selected from OH or SH;
   each G is independently selected from OH, halogen or O—[C(R₆)(R₇)]ₙ—[(C═O)ₘ—X^{G}]ⱼ—R₈;
   each R₆ and R₇ is, independently, H, halogen, C₁-C₆ alkyl or substituted C₁-C₆ alkyl;
   each X^{G} is O, S or N(E₁);
      R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
   E₁, E₂ and E₃ are each, independently, H, C₁-C₆ alkyl or substituted C₁-C₆ alkyl;
   n is from 1 to 6;
   m is 0 or 1;
   j is 0 or 1;
      each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
   Q₂ is O, S or NJ₃;
   each J₁, J₂ and J₃ is, independently, H or C₁-C₆ alkyl.

Embodiment 1731. The RNAi agent of claim 1730, wherein each G of Structure M within the RNAi sense oligonucleotide is selected from F or OMe.

Embodiment 1732. The RNAi agent of claim 1731, wherein one G is F and the other G is OMe.

Embodiment 1733. The RNAi agent of any of claims 1711 or 1728-1732, wherein the 5′-terminus of the RNAi sense oligonucleotide has structure N:

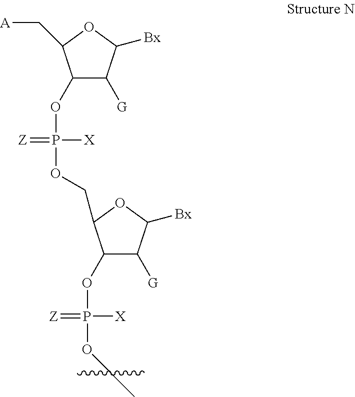

wherein:
A is selected from

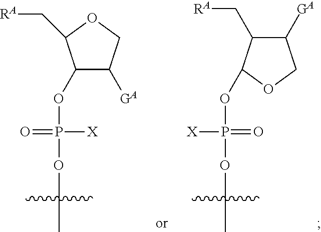

R^{A} is OH, OP(═O)OH, OP(═O)SH, a stabilized phosphate group or a mesyl phosphoramidate;
G^{A} is H, OH, OMe, MOE, or a halogen;
each Bx is an independently selected heterocyclic base moiety;
each X is independently selected from OH or SH;
each G is independently selected from OH, halogen or O—[C(R₆)(R₇)]ₙ-[(C═O)ₘ—X^{G}]ⱼ—R₈;
each R₆ and R₇ is, independently, H, halogen, C₁-C₆ alkyl or substituted C₁-C₆ alkyl;
each X^{G} is O, S or N(E₁);
   R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

Embodiment 1734. The RNAi agent of claim 1733, wherein each G within structure N of the RNAi sense oligonucleotide is selected from F or OMe.

Embodiment 1735. The RNAi agent of any of claims 1711, 1725-1727, or 1730-1734, wherein the 3′-terminus of the RNAi sense oligonucleotide has structure O:

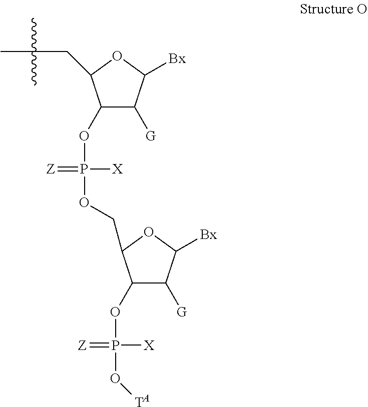

wherein:
T^{A} is selected from

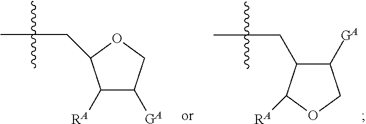

R^{A} G^{A} or R^{A} O
R^{A} is OH, OP(═O)OH, OP(═O)SH, or a stabilized phosphate group;
G^{A} is H, OH, OMe, MOE, or a halogen;
each Bx is an independently selected heterocyclic base moiety;
each X is independently selected from OH or SH;
each Z is selected from O, S, or NSO₂Me;
at least one Z is NSO₂Me;
each G is independently selected from OH, halogen or O—[C(R₆)(R₇)]ₙ[(C═O)ₘ—X^{G}]ⱼ—R₈;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

Embodiment 1736. The RNAi agent of claim 1735, wherein each G within structure O of the RNAi sense oligonucleotide is selected from F or OMe.

Embodiment 1737. The oligomeric compound or antisense agent of any of claims 1246-1456 or 1484-1724, comprising at least one modified oligonucleotide, wherein the nucleobase sequence of at least one modified oligonucleotide is complementary to a target nucleic acid.

Embodiment 1738. The modified oligonucleotide of claim 1737, wherein the nucleobase sequence of the modified oligonucleotide is at least 80% complementary to the target nucleic acid.

Embodiment 1739. The modified oligonucleotide of claim 1737, wherein the nucleobase sequence of the modified oligonucleotide is at least 85% complementary to the target nucleic acid.

Embodiment 1740. The modified oligonucleotide of claim 1737, wherein the nucleobase sequence of the modified oligonucleotide is at least 90% complementary to the target nucleic acid.

Embodiment 1741. The modified oligonucleotide of claim 1737, wherein the nucleobase sequence of the modified oligonucleotide is at least 95% complementary to the target nucleic acid.

Embodiment 1742. The modified oligonucleotide of claim 1737, wherein the nucleobase sequence of the modified oligonucleotide is 100% complementary to the target nucleic acid.

Embodiment 1743. The modified oligonucleotide of any of claims 1737-1742, wherein the target nucleic acid is a target RNA.

Embodiment 1744. The modified oligonucleotide of claim 1743, wherein the target RNA is selected from: an mRNA, a pre-mRNA, a microRNA, and a non-coding RNA.

Embodiment 1745. The modified oligonucleotide of claim 1744, wherein the target RNA is not a microRNA.

Embodiment 1746. The antisense agent comprising a modified oligonucleotide of any of claims 1-500, wherein the modified oligonucleotide is not complementary to miR-21.

Embodiment 1747. The antisense agent of any of claims 1246-1746, comprising a conjugate group.

Embodiment 1748. The antisense agent of claim 1747, wherein the conjugate group comprises at least one GalNAc.

Embodiment 1749. The antisense agent of claim 1747 or 1748, wherein the conjugate group comprises 1-5 linker-nucleosides.

Embodiment 1750. A pharmaceutical composition comprising the oligomeric compound of any of claims 1246-1694 or the antisense agent of any of claims 1695-1730 and a pharmaceutically acceptable carrier or diluent.

Embodiment 1751. A method comprising contacting a cell with the oligomeric compound, antisense agent, or pharmaceutical composition of any of claims 1246-1750.

Embodiment 1752. A method of modulating the amount or activity of a target nucleic acid in a cell, comprising contacting the cell with the oligomeric compound, antisense agent, or pharmaceutical composition of any of claims 1246-1750 and thereby modulating the amount or activity of the target nucleic acid.

Embodiment 1753. A method of modulating the amount or activity of a target nucleic acid in a cell, comprising contacting the cell with the oligomeric compound, antisense agent or pharmaceutical composition of any of claims 1246-1750.

Embodiment 1754. The method of claims 1751-1753, wherein the amount or activity of a target nucleic acid is reduced.

Embodiment 1755. The method of claims 1751-1753, wherein the amount or activity of a target nucleic acid is increased.

Embodiment 1756. The method of claim 1753, wherein the target nucleic acid comprises at least one translation suppression element and wherein the modified oligonucleotide is complementary to a target site within a translation suppression element region of the target nucleic acid.

Embodiment 1757. The method of claim 1756, wherein the translation suppression element region comprises at least one stem-loop structure.

Embodiment 1758. Use of the antisense agent or composition of any of claims 1246-1750 for treatment of a disease or condition.

Embodiment 1759. Use of the antisense agent or composition of any of claims 1246-1750 for a preparation of a medicament for treatment of a disease or condition.

Embodiment 1760. The oligomeric compound or antisense agent of any of claims 1246-1749, wherein the oligomeric compound or antisense agent is not an RNAi agent and the parent oligomeric compound or antisense agent is cytotoxic in vitro.

Embodiment 1761. The oligomeric compound or antisense agent of claim 1760, wherein the parent oligomeric compound or antisense agent is cytotoxic in a standard in vitro cytotoxicity assay.

Embodiment 1762. The oligomeric compound or antisense agent of claim 1760, wherein the oligomeric compound or antisense agent of any of claims 1246-1749is not cytotoxic in vitro.

Embodiment 1763. The oligomeric compound or antisense agent of any of claims 1760-1762, wherein the oligomeric compound or antisense agent of any of claims 1246-1749 is not cytotoxic in a standard in vitro cytotoxicity assay.

Embodiment 1764. The oligomeric compound or antisense agent of any of claims 1246-1749, wherein the antisense agent is not an siRNA agent and the parent antisense agent is hepatotoxic to the mouse.

Embodiment 1765. The oligomeric compound or antisense agent of claim 1764, wherein the mouse is a BALB/c mouse, wherein 50 mg/kg of the parent antisense agent is administered to the mouse, and wherein the plasma ALT level in the mouse is measured 72 hours following the administration of the parent antisense agent.

Embodiment 1766. The oligomeric compound or antisense agent of any of claims 1764-1765, wherein administration of 50 mg/kg of the oligomeric compound or antisense agent of any of claims 1246-1749 to a mouse is not hepatotoxic to the mouse.

Embodiment 1767. The oligomeric compound or antisense agent of any of claims 1246-1749, wherein the therapeutic index in a mouse of the antisense agent of any of claims 1246-1749 is increased relative to the therapeutic index of the parent antisense agent.

Embodiment 1768. The oligomeric compound or antisense agent of claim 1767, wherein the therapeutic index in a mouse of the antisense agent of claim 516 is at least two-fold greater than the therapeutic index of the parent antisense agent.

Embodiment 1769. The oligomeric compound or antisense agent of any of claims 1760-1768, wherein the parent oligomeric compound or antisense agent is identical to the antisense agent of any of claims 1246-1749, except that each internucleoside linkage of Formula XVII is replaced with a phosphorothioate internucleoside linkage in the parent antisense agent.

Embodiment 1770. The oligomeric compound or of any of claims 1760-1769, wherein the oligomeric compound or antisense agent is an RNAse H agent.

Embodiment 1771. The oligomeric compound or antisense agent of any of claims 1760-1769, wherein the oligomeric compound or antisense agent is a gapmer.

Embodiment 1772. The oligomeric compound or antisense agent of any of claims 1760-1769, wherein the oligomeric compound or antisense agent modulates splicing.

Embodiment 1773. The oligomeric compound or antisense agent of any of claims 1760-1769, wherein the oligomeric compound or antisense agent increases protein expression.

Embodiment 1774. The oligomeric compound or antisense agent of any of claims 1246-1749, wherein the oligomeric compound or antisense agent is an RNAi agent, and the parent RNAi agent is cytotoxic in vitro.

Embodiment 1775. The oligomeric compound or antisense agent of claim 1774, wherein the RNAi agent of any of claims 1246-1749 is not cytotoxic in vitro.

Embodiment 1776. The oligomeric compound or antisense agent of any of claims 1774-1775, wherein the oligomeric compound or antisense agent is an RNAi agent and the RNAi agent is not cytotoxic in a standard in vitro cytotoxicity assay.

Embodiment 1777. The oligomeric compound or antisense agent of any of claims 1246-1749, wherein the oligomeric compound or antisense agent is an RNAi agent and is hepatotoxic to the mouse.

Embodiment 1778. The RNAi agent of claim 1777, wherein the mouse is a BALB/c mouse, wherein 50 mg/kg of the parent RNAi agent is administered to the mouse, and wherein the plasma ALT level in the mouse is measured 72 hours following the administration of the parent RNAi agent.

Embodiment 1779. The RNAi agent of any of claims 1777-1778, wherein administration of 50 mg/kg of the RNAi agent to a mouse is not hepatotoxic to the mouse.

Embodiment 1780. The oligomeric compound or antisense agent of any of claims 1246-1749, which is an RNAi agent, wherein the therapeutic index in a mouse of the RNAi agent is increased relative to the therapeutic index of the parent RNAi agent.

Embodiment 1781. The RNAi agent of claim 1780, wherein the therapeutic index in a mouse of the RNAi agent of claim 535 is at least two-fold greater than the therapeutic index of the parent RNAi agent.

Embodiment 1782. The RNAi agent of any of claims 1771-1781, wherein the parent RNAi agent is identical to the oligomeric compound or antisense agent any of claims 1246-1749, except that each internucleoside linkage of Formula XVII is replaced with a phosphodiester internucleoside linkage in the parent RNAi agent.

Embodiment 1783. A method of designing an oligomeric compound or antisense agent comprising starting with a parent oligomeric compound or antisense agent or parent RNAi agent and changing the design of that compound in order to arrive at an oligomeric compound or antisense agent of any one of claims 1246-1749.

Embodiment 1784. A method of designing an oligomeric compound or an antisense agent comprising identifying an oligomeric compound or antisense agent or parent RNAi agent and changing the design of that parent oligomeric compound or antisense agent or parent RNAi agent to arrive at a second antisense agent, wherein the second oligomeric compound antisense agent is an oligomeric compound or antisense agent of any one of claims 1246-1749.

Embodiment 1785. A method of improving hepatotoxicity of an oligomeric compound or antisense agent comprising the steps of (i) identifying a parent oligomeric compound, parent antisense agent or parent RNAi agent that has plasma ALT levels above 300 units per liter in a mouse, and (ii) providing an oligomeric compound or antisense agent according to any one of claims 1246-1749.

Embodiment 1786. The method of claim 1785, wherein the method designs an oligomeric compound or antisense agent with improved therapeutic index relative to the parent oligomeric compound, parent antisense agent, or parent RNAi agent.

Embodiment 1787. The method of claim 1785, wherein the method designs an oligomeric compound or antisense agent with lower hepatotoxicity relative to the parent oligomeric compound, parent antisense agent or parent RNAi agent.

Embodiment 1788. The method of claim 1785, wherein the second oligomeric compound or antisense agent has an improved therapeutic index relative to the parent oligomeric compound, parent antisense agent or parent RNAi agent.

Embodiment 1789. The method of claim 1785, wherein the second oligomeric compound or antisense agent has reduced hepatotoxicity in a mouse relative to the parent oligomeric compound, parent antisense agent or parent RNAi agent.

Embodiment 1790. The method of claim 1785, wherein the oligomeric compound or antisense agent according to any one of claims 1246-1749 has improved therapeutic index relative to the parent oligomeric compound, parent antisense agent or parent RNAi agent.

Embodiment 1791. The method of claim 1785, wherein the oligomeric compound or antisense agent according to any one of claims 1246-1749 has reduced hepatotoxicity relative to the parent oligomeric compound, antisense agent or parent RNAi agent.

Embodiment 1792. A method comprising administering an oligomeric compound or antisense agent of any of claims 1246-1749 to a mouse and separately administering the parent oligomeric compound, parent antisense agent or parent RNAi agent of the antisense agent of any of claims 1246-1749 to a second mouse, wherein the therapeutic index of the antisense agent of any of claims 1246-1749 is improved relative to the therapeutic index of the parent antisense agent or parent RNAi agent.

### Certain Compounds

In certain embodiments, compounds described herein are oligomeric compounds (including oligomeric compounds that are antisense agents or portions thereof) comprising or consisting of oligonucleotides consisting of linked nucleosides and having at least one modified internucleoside linking group having Formula VIII or Formula XVII. Oligonucleotides may be unmodified oligonucleotides or may be modified oligonucleotides. Modified oligonucleotides comprise at least one modification relative to an unmodified oligonucleotide (i.e., comprise at least one modified nucleoside (comprising a modified sugar moiety, a stereo-non-standard nucleoside, and/or a modified nucleobase) and/or at least one modified internucleoside linkage). In certain embodiments, the modified internucleoside linkage is a modified internucleoside linking group having Formula VIII or Formula XVII. In certain embodiments, compounds described herein are oligomeric compounds (including oligomeric compounds that are antisense agents or portions thereof) having at least one modified internucleoside linking group having Formula XVII.

### I. Modifications

### A. Modified Nucleosides

Modified nucleosides comprise a stereo-non-standard nucleoside, or a modified sugar moiety, or a modified nucleobase, or any combination thereof.

### 1. Certain Modified Sugar Moieties

In certain embodiments, modified sugar moieties are stereo-non-standard sugar moieties. In certain embodiments, sugar moieties are substituted furanosyl stereo-standard sugar moieties. In certain embodiments, modified sugar moieties are bicyclic or tricyclic furanosyl sugar moieties. In certain embodiments, modified sugar moieties are sugar surrogates. Such sugar surrogates may comprise one or more substitutions corresponding to those of other types of modified sugar moieties.

a. Stereo-Non-Standard Sugar Moieties

In certain embodiments, modified sugar moieties are stereo-non-standard sugar moieties shown in Formula I, Formula II, Formula III, Formula IV, Formula V, Formula VI, and Formula VII:

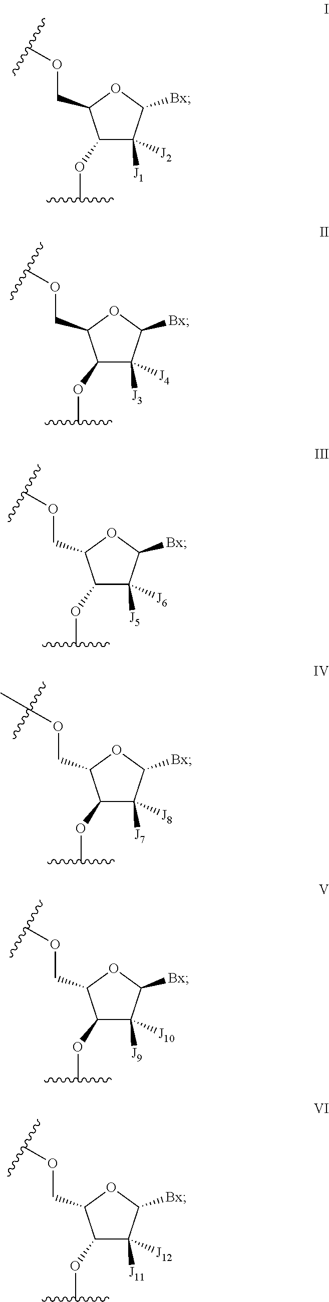

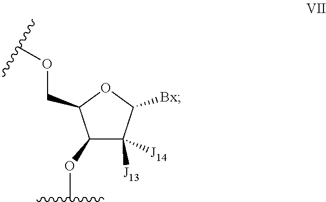

wherein
one of J₁ and J₂ is H and the other of J₁ and J₂ is selected from H, OH, F, OCH₃, OCH₂CH₂OCH₃, O—C₁-C₆ alkoxy, and SCH₃;
one of J₃ and J₄ is H and the other of J₃ and J₄ is selected from H, OH, F, OCH₃, OCH₂CH₂OCH₃, O—C₁-C₆ alkoxy, and SCH₃; and wherein
one of J₅ and J₆ is H and the other of J₅ and J₆ is selected from H, OH, F, OCH₃, OCH₂CH₂OCH₃, O—C₁-C₆ alkoxy, and SCH₃; and wherein
one of J₇ and J₈ is H and the other of J₇ and J₈ is selected from H, OH, F, OCH₃, OCH₂CH₂OCH₃, O—C₁-C₆ alkoxy, and SCH₃; and wherein
one of J₉ and J₁₀ is H and the other of J₉ and J₁₀ is selected from H, OH, F, OCH₃, OCH₂CH₂OCH₃, O—C₁-C₆ alkoxy, and SCH₃; and wherein
one of J₁₁ and J₁₂ is H and the other of J₁₁ and J₁₂ is selected from H, OH, F, OCH₃, OCH₂CH₂OCH₃, O—C₁-C₆ alkoxy, and SCH₃; and wherein
one of J₁₃ and J₁₄ is H and the other of J₁₃ and J₁₄ is selected from H, OH, F, OCH₃, OCH₂CH₂OCH₃, O—C₁-C₆ alkoxy, and SCH₃; and
   Bx is a is a heterocyclic base moiety.

Certain stereo-non-standard sugar moieties have been previously described in, e.g., Seth et al., WO2020/072991 and Seth et al., WO2019/157531, both of which are incorporated by reference herein in their entirety.

b. Substituted Stereo-Standard Sugar Moieties

In certain embodiments, modified sugar moieties are substituted stereo-standard furanosyl sugar moieties comprising one or more acyclic substituent, including but not limited to substituents at the 2′, 3′, 4′, and/or 5′ positions. In certain embodiments, the furanosyl sugar moiety is a ribosyl sugar moiety. In certain embodiments one or more acyclic substituent of substituted stereo-standard sugar moieties is branched. Examples of 2′-substituent groups suitable for substituted stereo-standard sugar moieties include but are not limited to: 2′-F, 2′-OCH₃ (“2′-OMe” or “2′-O-methyl”), and 2′-O(CH₂)₂OCH₃ (“2′-MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, C₁-C₁₀ alkyl, C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)2SCH₃, O(CH₂)20N(Rₘ)(Rₙ) or OCH₂C(═O)—N(Rₘ)(Rₙ), where each Rₘ and R. is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 3′-substituent groups include 3′-methyl (see Frier, et al., The ups and downs of nucleic acid duplex stability: structure-stability studies on chemically-modified DNA:RNA duplexes. Nucleic Acids Res., 25, 4429-4443, 1997.) Examples of 4′-substituent groups suitable for substituted stereo-standard sugar moieties include but are not limited to alkoxy (e.g., methoxy), alkyl, and those described in Manoharan et al., WO 2015/106128. Examples of 5′-substituent groups suitable for substituted stereo-standard sugar moieties include but are not limited to: 5′-methyl (R or S), 5′-allyl, 5′-ethyl, 5′-vinyl, and 5′-methoxy. In certain embodiments, non-bicyclic modified sugars comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836. 2′,4′-difluoro modified sugar moieties have been described in Martinez-Montero, et al., Rigid 2′,4′-difluororibonucleosides: synthesis, conformational analysis, and incorporation into nascent RNA by HCV polymerase. J. Org. Chem., 2014, 79:5627-5635. Modified sugar moieties comprising a 2′-modification (OMe or F) and a 4′-modification (OMe or F) have also been described in Malek-Adamian, et al., J. Org. Chem, 2018, 83: 9839-9849.

In certain embodiments, a 2′-substituted stereo-standard nucleoside comprises a sugar moiety comprising a non-bridging 2′-substituent group selected from: F, NH₂, N₃, OCF₃, OCH₃, SCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(=O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted stereo-standard nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted stereo-standard nucleoside comprises a sugar moiety comprising a 2′-substituent group selected from: F, OCH₃, and OCH₂CH₂OCH₃.

In certain embodiments, the 4′ O of 2′-deoxyribose can be substituted with a S to generate 4′-thio DNA (see Takahashi, et al., Nucleic Acids Research 2009, 37: 1353-1362). This modification can be combined with other modifications detailed herein. In certain such embodiments, the sugar moiety is further modified at the 2′ position. In certain embodiments the sugar moiety comprises a 2′-fluoro. A thymidine with this sugar moiety has been described in Watts, et al., J Org. Chem. 2006, 71(3): 921-925 (4′-S-fluoro5-methylarauridine or FAMU).

c. Bicyclic Nucleosides

Certain nucleosides comprise modified sugar moieties that comprise a bridging sugar substituent that forms a second ring resulting in a bicyclic sugar moiety. In certain such embodiments, the bicyclic sugar moiety comprises a 4′ to 2′ bridge between the 4′ and the 2′ furanose ring atoms. In certain such embodiments, the furanose ring is a ribose ring. Examples of sugar moieties comprising such 4′ to 2′ bridging sugar substituents include but are not limited to bicyclic sugars comprising: 4′-CH₂-2′, 4′—(CH₂)2-2′, 4′—(CH₂)3-2′, 4′—CH₂—O-2′ (“LNA”), 4′-CH₂—S-2′, 4′—(CH₂)₂—O-2′ (“ENA”), 4′-CH(CH₃)—O-2′ (referred to as “constrained ethyl” or “cEt” when in the S configuration), 4′-CH₂—O—CH₂-2′, 4′—CH₂—N(R)-2′, 4′—CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem.,2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′—C(RₐR_{b})—O—N(R)-2′, 4′—CH₂—O—N(R)-2′, and 4′-CH₂—N(R)—O-2′, wherein each R, Ra, and R_{b} is, independently, H, a protecting group, or C₁-C₁₂ alkyl (see, e.g. Imanishi et al., U.S. Pat. No. 7,427,672), 4′-C(═O)—N(CH₃)2-2′, 4′—C(═O)—N(R)₂-2′, 4′—C(═S)—N(R)₂-2′ and analogs thereof (see, e.g., Obika et al., WO2011052436A1, Yusuke, WO2017018360A1).

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2017, 129, 8362-8379; Elayadi et al.; Christiansen, et al., J. Am. Chem. Soc. 1998, 120, 5458-5463; Wengel et a., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. patenttent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the P-D configuration.

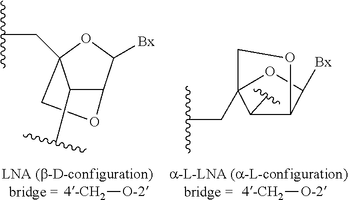

α-L-methyleneoxy (4′-CH₂—O-2′) or α-L-LNA bicyclic nucleosides have been incorporated into antisense oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA) are identified in exemplified embodiments herein, they are in the P-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

The term “substituted” following a position of the furanosyl ring, such as “2′-substituted” or “2′-4′-substituted”, indicates that is the only position(s) having a substituent other than those found in unmodified sugar moieties in oligonucleotides.

d. Sugar Surrogates

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), altritol nucleic acid (“ANA”), mannitol nucleic acid (“MNA”) (see, e.g., Leumann, C J. Bioorg. & Med. Chem. 2002, 10, 841-854), fluoro HNA (“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran).

In certain embodiments, sugar surrogates comprise rings having no heteroatoms. For example, nucleosides comprising bicyclo [3.1.0]-hexane have been described (see, e.g., Marquez, et al., J. Med. Chem. 1996, 39:3739-3749).

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate comprising the following structure:

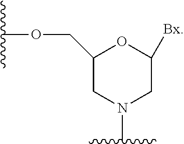

In certain embodiments, morpholinos may be modified, for example by adding or altering various substituent groups from the above morpholino structure. Such sugar surrogates are referred to herein as “modified morpholinos.” In certain embodiments, morpholino residues replace a full nucleotide, including the internucleoside linkage, and have the structures shown below, wherein Bx is a heterocyclic base moiety.

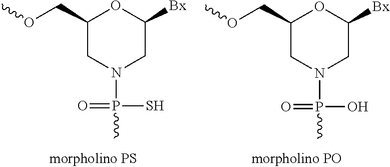

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013,11,5853-5865), glycol nucleic acid (“GNA”, see Schlegel, et al., J. Am. Chem. Soc. 2017, 139:8537-8546) and nucleosides and oligonucleotides described in Manoharan et al., WO2011/133876. In certain embodiments, acyclic sugar surrogates are selected from:

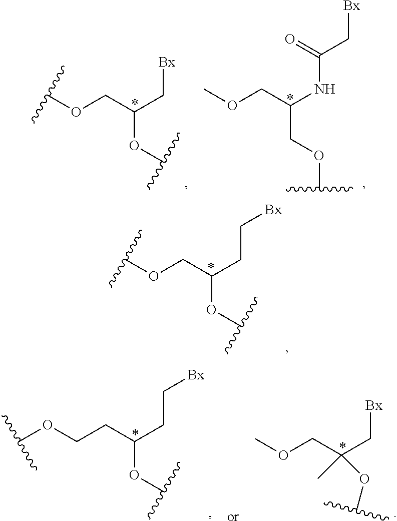

Many other bicyclic and tricyclic sugar and sugar surrogate ring systems are known in the art that can be used in modified nucleosides. Certain such ring systems are described in Hanessian, et al., J Org. Chem., 2013, 78: 9051-9063 and include bcDNA and tcDNA. Modifications to bcDNA and tcDNA, such as 6′-fluoro, have also been described (Dogovic and Leumann, J. Org. Chem., 2014, 79: 1271-1279).

e. Conjugated Nucleosides and Terminal Groups

In certain embodiments, modified sugar moieties comprise a conjugate group and/or a terminal group. Modified sugar moieties are linked to conjugate groups through a conjugate linker. In certain embodiments, modified furanosyl sugar moieties comprise conjugate groups attached at the 2′, 3′, or 5′ positions. In certain embodiments, the 3′-most sugar moiety of the nucleoside is modified with a conjugate group or a terminal group. In certain embodiments, the 5′-most sugar moiety of the nucleoside is modified with a conjugate group or a terminal group. In certain embodiments, a sugar moiety near the 3′ end of the nucleoside is modified with a conjugate group. In certain embodiments, a sugar moiety near the 5′ end of the nucleoside is modified with a conjugate group.

Examples of terminal groups include but are not limited to conjugate groups, capping groups, phosphate moieties, protecting groups, modified or unmodified nucleosides, and two or more nucleosides that are independently modified or unmodified.

In certain embodiments, terminal groups at the 5′-terminus comprise a stabilized phosphate group. In certain such embodiments, the phosphorus atom of the stabilized phosphate group is attached to the 5′-terminal nucleoside through a phosphorus-carbon bond. In certain embodiments, the carbon of that phosphorus-carbon bond is in turn bound to the 5′-position of the nucleoside.

In certain embodiments, the oligonucleotide comprises a 5′-stabilized phosphate group having the following formula:

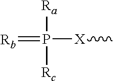

wherein:
   Rₐ and R_{c} are each, independently, OH, SH, C₁-C₆ alkyl, substituted C₁-C₆ alkyl, C₁-C₆ alkoxy, substituted C₁-C₆ alkoxy, amino or substituted amino;
   R_{b} is O or S;
   X is substituted or unsubstituted C; and wherein X is attached to the 5′-terminal nucleoside. In certain embodiments, X is bound to an atom at the 5′-position of the 5′-terminal nucleoside. In certain such embodiments, the 5′-atom is a carbon and the bond between X and the 5′-carbon of the 5′-terminal nucleoside is a carbon-carbon single bond. In certain embodiments, it is a carbon-carbon double bond. In certain embodiments, it is a carbon-carbon triple bond. In certain embodiments, the 5′-carbon is substituted. In certain embodiments, X is substituted. In certain embodiments, X is unsubstituted.

In certain embodiments, the oligonucleotide comprises a 5′-stabilized phosphate group having the following formula:

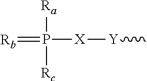

wherein:
   Rₐ and R_{c} are each, independently, OH, SH, C₁-C₆ alkyl, substituted C₁-C₆ alkyl, C₁-C₆ alkoxy, substituted C₁-C₆ alkoxy, amino or substituted amino;
   R_{b} is O or S;
   X is substituted or unsubstituted C;
   Y is selected from C, S, and N. In certain embodiments, Y is substituted or unsubstituted C. The bond between X and Y may be a single-, double-, or triple-bond.

Certain 5′-stabilized phosphate groups have been previously described; see, e.g., Prakash et al., WO2011/139699 and Prakash et al., WO2011/139702, hereby incorporated by reference herein in their entirety.

In certain embodiments, the stabilized phosphate group is 5′-vinyl phosphonate or 5′-cyclopropyl phosphonate.

In certain embodiments, a terminal group at the 5′-terminus is a 5′-mesyl phosphoramidate, having formula XXI:

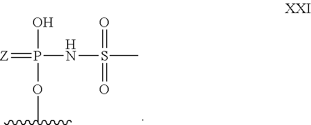

wherein Z is O or S.

In certain embodiments, a terminal group at the 5′-terminus is a 5′-mesyl phosphoramidate, having formula XXII:

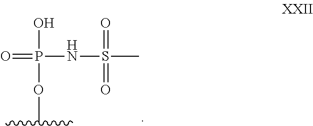 [SMILES: *OP(=O)(O)NS(C)(=O)=O]

### 2. Modified Nucleobases

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C═C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deazaadenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443. In certain embodiments, modified nucleosides comprise double-headed nucleosides having two nucleobases. Such compounds are described in detail in Sorinas et al., J. Org. Chem, 2014 79: 8020-8030.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

In certain embodiments, compounds comprise or consist of a modified oligonucleotide complementary to a target nucleic acid comprising one or more modified nucleobases. In certain embodiments, the modified nucleobase is 5-methylcytosine. In certain embodiments, each cytosine is a 5-methylcytosine.

### B. Modified Internucleoside Linkages

a. Internucleoside Linkages of Formula VIII and XVII

In certain embodiments, antisense agents, oligomeric compounds, and modified oligonucleotides described herein having one or more modified internucleoside linkages having Formula VIII or Formula XVII are selected over compounds lacking such internucleoside linkages having Formula VIII or Formula XVII because of one or more desirable properties. In certain embodiments, antisense agents, oligomeric compounds, and modified oligonucleotides described herein having one or more modified internucleoside linkages having Formula VIII or Formula XVII have enhanced cellular uptake. In certain embodiments, antisense agents, oligomeric compounds, and modified oligonucleotides described herein having one or more modified internucleoside linkages having Formula VIII or Formula XVII have enhanced affinity for target nucleic acids. In certain embodiments, antisense agents, oligomeric compounds, and modified oligonucleotides described herein having one or more modified internucleoside linkages having Formula VIII or Formula XVII have increased stability in the presence of nucleases. In certain embodiments, antisense agents, oligomeric compounds, and modified oligonucleotides described herein having one or more modified internucleoside linkages having Formula VIII or Formula XVII have enhanced cellular uptake, enhanced affinity for target nucleic acids, and increased stability in the presence of nucleases. In certain embodiments, antisense agents, oligomeric compounds, and modified oligonucleotides described herein having one or more modified internucleoside linkages having Formula VIII or Formula XVII have enhanced bioavailability. In certain embodiments, antisense agents, oligomeric compounds, and modified oligonucleotides described herein having one or more modified internucleoside linkages having Formula VIII or Formula XVII have enhanced RNase H activity. In certain embodiments, antisense agents, oligomeric compounds, and modified oligonucleotides described herein having one or more modified internucleoside linkages having Formula VIII or Formula XVII have enhanced RNAi activity. In certain embodiments, antisense agents, oligomeric compounds, and modified oligonucleotides described herein having one or more modified internucleoside linkages having Formula VIII or Formula XVII have enhanced CRISPR activity. In certain embodiments, antisense agents, oligomeric compounds, and modified oligonucleotides described herein having one or more modified internucleoside linkages having Formula VIII or Formula XVII have reduced interactions with certain proteins. In certain embodiments, antisense agents, oligomeric compounds, and modified oligonucleotides described herein having one or more modified internucleoside linkages having Formula VIII or Formula XVII have increased interactions with certain proteins.

In certain embodiments, oligomeric compounds (including oligomeric compounds that are antisense agents or portions thereof) comprise or consist of a modified oligonucleotide complementary to a target nucleic acid comprising one or more modified internucleoside linkages having Formula VIII:

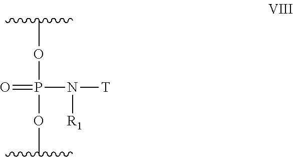

wherein independently for each internucleoside linking group of the oligomeric compound having Formula VIII:
R₁ is selected from H, C₁-C₆ alkyl, and substituted C₁-C₆ alkyl; and
T is selected from SO₂R₂, C(═O)R₃, and P(═O)R₄R₅, wherein:
   R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, and substituted C₁-C₆ alkyl;
   R₃ is selected from an aryl, a substituted aryl, CH₃, N(CH₃)₂, and OCH₃;
   R₄ is selected from OCH₃, OH, C₁-C₆ alkyl, and substituted C₁-C₆ alkyl; and
R₅ is selected from OCH₃, OH, C₁-C₆ alkyl, and substituted C₁-C₆ alkyl.

In certain embodiments, oligomeric compounds (including oligomeric compounds that are antisense agents or portions thereof) comprise or consist of a modified oligonucleotide complementary to a target nucleic acid comprising one or more modified internucleoside linkages having Formula VIII:

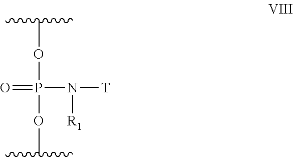

wherein independently for each internucleoside linking group of the oligomeric compound having Formula VIII:
R₁ is selected from H, C₁-C₆ alkyl, and substituted C₁-C₆ alkyl; and
T is selected from SO₂R₂, C(═O)R₃, and P(═O)R₄R₅, wherein:
   R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, a C₁-C₆ alkyl, and a substituted C₁-C₆ alkyl;
   R₃ is selected from an aryl, a substituted aryl, CH₃, N(CH₃)₂, and OCH₃;
   R₄ is selected from OCH₃, OH, C₁-C₆ alkyl, and substituted C₁-C₆ alkyl; and
   R₅ is selected from OCH₃, OH, C₁-C₆ alkyl, and substituted C₁-C₆ alkyl;
provided that if R₁ is H, then T is not:

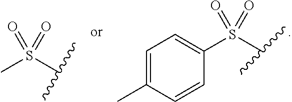

In certain embodiments, oligomeric compounds (including oligomeric compounds that are antisense agents or portions thereof) comprise or consist of a modified oligonucleotide complementary to a target nucleic acid comprising one or more modified internucleoside linkages having Formula XVII:

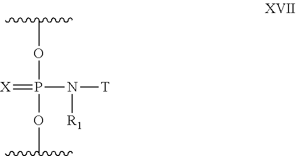

wherein independently for each internucleoside linking group of the oligomeric compound having Formula XVII:
X is selected from O or S;
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group; and
R₅ is selected from OCH₃, OH, C₁-C₆ alkyl, and substituted C₁-C₆ alkyl.

In certain embodiments, compounds comprise or consist of a modified oligonucleotide complementary to a target nucleic acid comprising one or more modified internucleoside linkages having Formula IX:

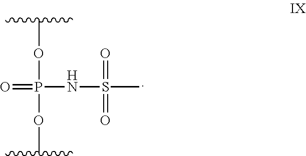 [SMILES: *OP(=O)(NS(C)(=O)=O)O*]

In certain embodiments, compounds comprise or consist of a modified oligonucleotide complementary to a target nucleic acid comprising one or more modified internucleoside linkages having Formula XX:

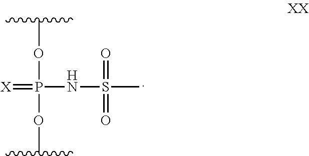

wherein independently for each internucleoside linking group of the oligomeric compound having Formula XX, X is selected from O or S.
b. Other Internucleoside Linkages

In certain embodiments, antisense agents, oligomeric compounds, and modified oligonucleotides comprise or consist of a modified oligonucleotide complementary to a target nucleic acid comprising one or more modified internucleoside linkages. In certain embodiments, the modified internucleoside linkages are phosphorothioate linkages. In certain embodiments, each internucleoside linkage of an antisense compound is a phosphorothioate internucleoside linkage.

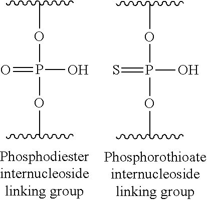

In certain embodiments, nucleosides of modified oligonucleotides may be linked together using any internucleoside linkage. The two main classes of internucleoside linkages are defined by the presence or absence of a phosphorus atom. Representative phosphorus-containing internucleoside linkages include unmodified phosphodiester internucleoside linkages, modified phosphotriesters such as THP phosphotriester and isopropyl phosphotriester, phosphonates such as methylphosphonate, isopropyl phosphonate, isobutyl phosphonate, and phosphonoacetate, phosphoramidates, phosphorothioate, and phosphorodithioate (“HS—P═S”). Representative non-phosphorus containing internucleoside linkages include but are not limited to methylenemethylimino (—CH₂—N(CH₃)—O—CH₂—), thiodiester, thionocarbamate (—O—C(═O)(NH)—S—); siloxane (—O—SiH₂—O—); formacetal, thioacetamido (TANA), alt-thioformacetal, glycine amide, and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. Methods of preparation of phosphorous-containing and non-phosphorous-containing internucleoside linkages are well known to those skilled in the art.

Neutral internucleoside linkages include, without limitation, phosphotriesters, phosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

c. Chiral Internucleoside Linkages

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. All phosphorothioate linkages described herein are stereorandom unless otherwise specified. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

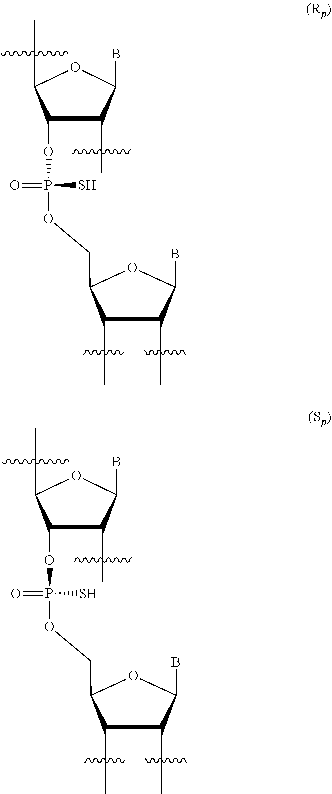

Unless otherwise indicated, chiral internucleoside linkages of modified oligonucleotides described herein can be stereorandom or in a particular stereochemical configuration.

In certain embodiments, an internucleoside linkage of Formula XVII may comprise a chiral center. An internucleoside linkage of Formula XVIII (XVII where X is S) comprises a chiral center. In certain embodiments, modified oligonucleotides comprise chiral linkages of Formula XVIII, illustrated below as XVIIIa and XVIIIb.

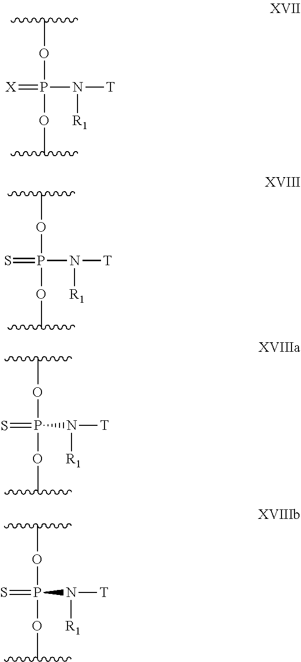

d. Alternatives to 5′ to 3′ Internucleoside Linkages

In certain embodiments, nucleic acids can be linked 2′ to 5′ rather than the standard 3′ to 5′ linkage. Such a linkage is illustrated below.

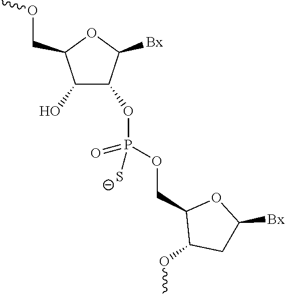

In certain embodiments, nucleosides can be linked by vicinal 2′, 3′-phosphodiester bonds. In certain such embodiments, the nucleosides are threofuranosyl nucleosides (TNA; see Bala, et al., J Org. Chem. 2017, 82:5910-5916). A TNA linkage is shown below.

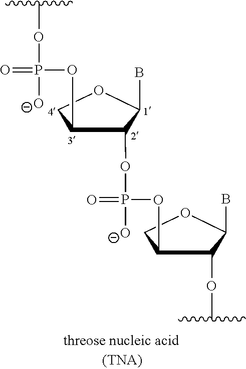

Additional modified linkages include α,β-D-CNA type linkages and related conformationally-constrained linkages, shown below. Synthesis of such molecules has been described previously (see Dupouy, et. al., Agnew. Chem. Int. Ed. Engl., 2014, 45: 3623-3627; Borsting, et al. Tetrahedron, 2004, 60:10955-10966; Ostergaard, et. al., ACS Chem. Biol. 2014, 9: 1975-1979; Dupouy, et al., Eur. J Org. Chem., 2008, 1285-1294; Matinez, et al., PLoS One, 2011, 6:e25510; Dupouy, et al., Eur. J. Org. Chem., 2007, 5256-5264; Boissoinnet, et al. New J. Chem., 2011, 35: 1528-1533.)

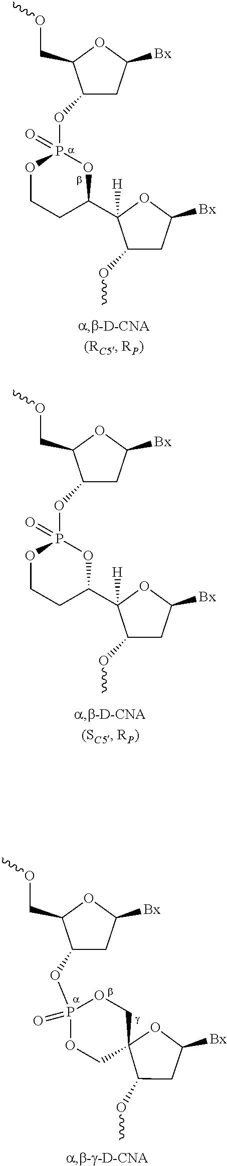

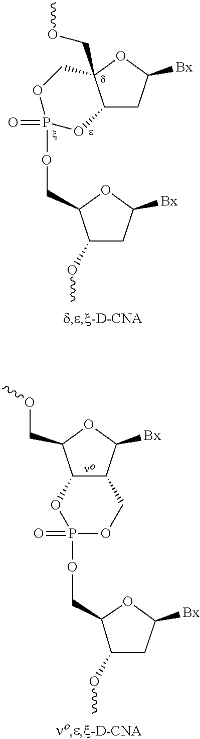

e. Linkages Having Conjugate Groups

In certain embodiments, an internucleoside linking group may comprise a conjugate group. In certain embodiments, an internucleoside linking group of Formula XVII comprises a conjugate group. In certain embodiments, the conjugate group of a modified oligonucleotide may be attached to the remainder of the modified oligonucleotide through a modified internucleoside having Formula XVII:

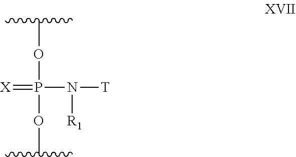

wherein T comprises a conjugate group. In certain embodiments, T is selected from SO₂R₂, C(═O)R₃, and P(═O)R₄R₅, wherein R₂, R₃, or R₄ is a conjugate group. In certain embodiments, the conjugate group comprises a cell-targeting moiety. In certain embodiments, the conjugate group comprises a carbohydrate or carbohydrate cluster. In certain embodiments, the conjugate group comprises GalNAc. In certain embodiments, the conjugate group comprises a lipid.

In certain embodiments, the conjugate group comprises C₁₀-C₂₀ alkyl. In certain embodiments, the conjugate group comprises C₁₆ alkyl.

In certain embodiments, the internucleoside linking group comprising a conjugate group has Formula XIX:

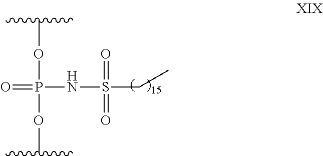

### II. Certain Motifs

In certain embodiments, antisense agents, oligomeric compounds, and modified oligonucleotides described herein comprise or consist of oligonucleotides. Modified oligonucleotides can be described by their motif, e.g. a pattern of unmodified and/or modified sugar moieties, nucleobases, and/or internucleoside linkages. In certain embodiments, modified oligonucleotides comprise one or more stereo-non-standard nucleosides. In certain embodiments, modified oligonucleotides comprise one or more stereo-standard nucleosides. In certain embodiments, modified oligonucleotides comprise one or more modified nucleoside comprising a modified sugar. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns or motifs of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### A. Certain Sugar Motifs

In certain embodiments, antisense agents, oligomeric compounds, and modified oligonucleotides described herein comprise or consist of oligonucleotides. In certain embodiments, oligonucleotides comprise one or more type of modified sugar and/or unmodified sugar moiety arranged along the oligonucleotide or region thereof in a defined pattern or sugar motif. In certain instances, such sugar motifs include without limitation any of the sugar modifications discussed herein.

In certain embodiments, a modified oligonucleotide comprises or consists of a gapmer. The sugar motif of a gapmer defines the regions of the gapmer: 5′-region, central region (gap), and 3′-region. The central region is linked directly to the 5′-region and to the 3′-region with no nucleosides intervening. The central region is a deoxy region. The nucleoside at the first position (position 1) from the 5′-end of the central region and the nucleoside at the last position of the central region are adjacent to the 5′-region and 3′-region, respectively, and each comprise a sugar moiety independently selected from a 2′-deoxyfuranosyl sugar moiety or a sugar surrogate. In certain embodiments, the nucleoside at position 1 of the central region and the nucleoside at the last position of the central region are DNA nucleosides, selected from stereo-standard DNA nucleosides or stereo-non-standard DNA nucleosides having any of formulas I-VII, wherein each J is H. In certain embodiments, the nucleoside at the first and last positions of the central region adjacent to the 5′ and 3′ regions are stereo-standard DNA nucleosides. Unlike the nucleosides at the first and last positions of the central region, the nucleosides at the other positions within the central region may comprise a 2′-substituted furanosyl sugar moiety or a substituted stereo-non-standard sugar moiety or a bicyclic sugar moiety. In certain embodiments, each nucleoside within the central region supports RNase H cleavage. In certain embodiments, a plurality of nucleosides within the central region support RNase H cleavage.

Herein, the lengths (number of nucleosides) of the three regions of a gapmer may be provided using the notation [#of nucleosides in the 5′-region]- [#of nucleosides in the central region]- [#of nucleosides in the 3′-region]. Thus, a 3-10-3 gapmer consists of 3 linked nucleosides in each of the 3′ and 5′ regions and 10 linked nucleosides in the central region. Where such nomenclature is followed by a specific modification, that modification is the modification of each sugar moiety of each 5′ and 3′-region and the central region nucleosides comprise stereo-standard DNA sugar moieties. Thus, a 5-10-5 MOE gapmer consists of 5 linked nucleosides each comprising 2′-MOE-stereo-standard sugar moieties in the 5′-region, 10 linked nucleosides each comprising a stereo-standard DNA sugar moiety in the central region, and 5 linked nucleosides each comprising 2′-MOE-stereo-standard sugar moieties in the 3′-region. A 5-10-5 MOE gapmer having a substituted stereo-non-standard nucleoside at position 2 of the gap has a gap of 10 nucleosides wherein the 2^{nd} nucleoside of the gap is a substituted stereo-non-standard nucleoside rather than the stereo-standard DNA nucleoside. Such oligonucleotide may also be described as a 5-1-1-8-5 MOE/substituted stereo-non-standard/MOE gapmer. A 3-10-3 cEt gapmer consists of 3 linked nucleosides each comprising a cEt in the 5′-region, 10 linked nucleosides each comprising a stereo-standard DNA sugar moiety in the central region, and 3 linked nucleosides each comprising a cEt in the 3′-region. A 3-10-3 cEt gapmer having a substituted stereo-non-standard nucleoside at position 2 of the gap has a gap of 10 nucleoside wherein the 2^{nd} nucleoside of the gap is a substituted stereo-non-standard nucleoside rather than the stereo-standard DNA nucleoside. Such oligonucleotide may also be described as a 3-1-1-8-3 cEt/substituted stereo-non-standard/cEt gapmer.

The sugar motif of a 3-10-3 cEt gapmer may also be denoted by the notation kkk-d(10)-kkk, wherein each “k” represents a cEt and each “d” represents a 2′-β-D-deoxyribosyl sugar moiety. This sugar motif is independent of the nucleobase sequence, the internucleoside linkage motif, and any nucleobase modifications. A 5-10-5 MOE gapmer may be denoted by the notation eeeee-d(10)-eeeee or e(5)-d(10)-e(5), wherein each “e” represents a 2′-MOE-$-D-ribofuranosyl sugar moiety, and each “d” represents a 2′-β-D-deoxyribosyl sugar moiety.

In certain embodiments, each nucleoside of a modified oligonucleotide, or portion thereof, comprises a 2′-substituted sugar moiety, a bicyclic sugar moiety, a sugar surrogate, or a 2′-deoxyribosyl sugar moiety. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, PNA, THP, and F-HNA.

In certain embodiments, modified oligonucleotides comprise at least 12, at least 13, at least 14, at least 15, at least 16, at least 17, at least 18, at least 19, or at least 20 nucleosides comprising a modified sugar moiety. In certain embodiments, the modified sugar moiety is selected independently from a 2′-substituted sugar moiety, a bicyclic sugar moiety, or a sugar surrogate. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, THP, and F-HNA.

In certain embodiments, each nucleoside of a modified oligonucleotide comprises a modified sugar moiety (“fully modified oligonucleotide”). In certain embodiments, each nucleoside of a fully modified oligonucleotide comprises a 2′-substituted sugar moiety, a bicyclic sugar moiety, or a sugar surrogate. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, THP, and F-HNA. In certain embodiments, each nucleoside of a fully modified oligonucleotide comprises the same modified sugar moiety (“uniformly modified sugar motif”). In certain embodiments, the uniformly modified sugar motif is 7 to 20 nucleosides in length. In certain embodiments, each nucleoside of the uniformly modified sugar motif comprises a 2′-substituted sugar moiety, a bicyclic sugar moiety, or a sugar surrogate. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, THP, and F-HNA. In certain embodiments, modified oligonucleotides having at least one fully modified sugar motif may also comprise at least 1, at least 2, at least 3, or at least 4 2′-deoxyribonucleosides.

### B. Certain Nucleobase Motifs

In certain embodiments antisense agents, oligomeric compounds, and modified oligonucleotides described herein comprise or consist of oligonucleotides. In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, one nucleoside comprising a modified nucleobase is in the central region of a modified oligonucleotide. In certain such embodiments, the sugar moiety of said nucleoside is a 2′-β-D-deoxyribosyl moiety. In certain such embodiments, the modified nucleobase is selected from: 5-methyl cytosine, 2-thiopyrimidine, 2-thiothymine, 6-methyladenine, inosine, pseudouracil, or 5-propynepyrimidine.

### C. Certain Internucleoside Linkage Motifs

In certain embodiments, antisense agents, oligomeric compounds, and modified oligonucleotides described herein comprise or consist of oligonucleotides. In certain embodiments, oligonucleotides comprise modified and/or unmodified internucleoside linkages arranged along the oligonucleotide or region thereof in a defined pattern or motif. In certain embodiments, the modified internucleoside linkages are internucleoside linking groups having Formula VIII. In certain embodiments, some or all of the internucleoside linkages in the 5′-region and 3′-region are modified internucleoside linkages having Formula VIII or Formula XVII. In certain embodiments, the terminal internucleoside linkages are modified internucleoside linkages having Formula VIII or Formula XVII. In certain embodiments, the internucleoside linkage motif comprises at least one phosphodiester internucleoside linkage in at least one of the 5′-region and the 3′-region, and at least one modified internucleoside linkage having Formula VIII or Formula XVII. In certain embodiments, the internucleoside linkage motif comprises at least one phosphorothioate internucleoside linkage in at least one of the 5′-region and the 3′-region, and at least one modified internucleoside linkage having Formula VIII or Formula XVII.

In certain embodiments, modified oligonucleotides comprise at least one region having Structure A:

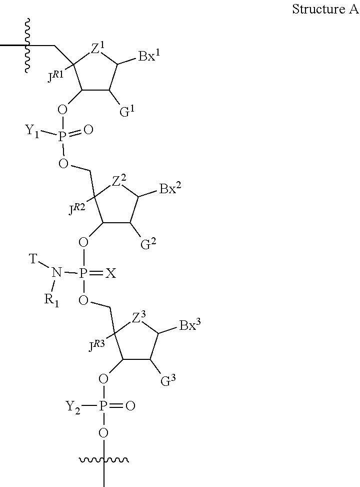

wherein:
each Bx is a heterocyclic base moiety;
X is selected from O or S;
each of Y₁ and Y₂ is independently selected from OH or SH;
each of Z¹, Z², and Z³ are independently selected from —(CH₂)ₚ—X^{Z}—(CH₂)_{q}—, wherein p is 0 or 1, q is 0 or 1, and X^{Z} is O, S, or N(E₁);
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group;
R₅ is selected from OCH₃, OH, C₁-C₆ alkyl, and substituted C₁-C₆ alkyl;
either J^{R1} and G¹ form a J^{R1} to G¹ bridge, or J^{R1} is H and G¹ is selected from H, OH, halogen or O—[C(R₆)(R₇)]ⱼ—[(C═O)ₘ—X^{G}]ⱼ—R₈;
either J^{R2} and G² form a J^{R2} and G² bridge, or J^{R2} is H and G² is selected from H, OH, halogen or O—[C(R₆)(R₇)]ₙ—[(C═O)ₘ—X^{G}]—R₈;
either J^{R3} and G³ form a J^{R3} and G³ bridge, or J^{R3} is H and G³ is selected from H, OH, halogen or O—[C(R₆)(R₇)]ₙ—[(C═O)ₘ—X^{G}]—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

In certain embodiments, modified oligonucleotides comprise at least one region having Structure B:

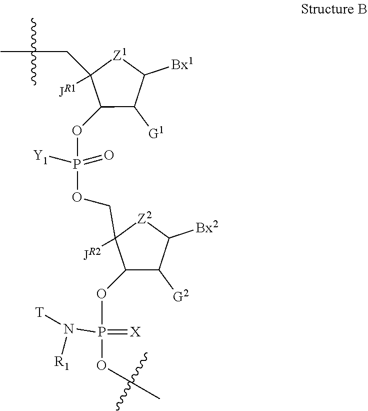

wherein:
   each Bx is a heterocyclic base moiety;
X is selected from O or S;
each of Y₁ and Y₂ is independently selected from OH or SH;
each of Z¹ and Z² are independently selected from —(CH₂)ₚ—X^{Z}—(CH₂)_{q}, wherein p is 0 or 1, q is 0 or 1, and X^{Z} is O, S, or N(E₁);
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group;
R₅ is selected from OCH₃, OH, C₁-C₆ alkyl, and substituted C₁-C₆ alkyl;
either J^{R1} and G¹ form a J^{R1} to G¹ bridge, or J^{R1} is H and G¹ is selected from H, OH, halogen or O—[C(R₆)(R₇)]—[(C═O)ₘ—X^{G}]ⱼ—R₈;
either J^{R2} and G² form a J^{R2} and G² bridge, or J^{R2} is H and G² is selected from H, OH, halogen or O—[C(R₆)(R₇)]ₙ—[(C═O)ₘ—X^{G}]—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

In certain embodiments, modified oligonucleotides comprise at least one region having Structure C:

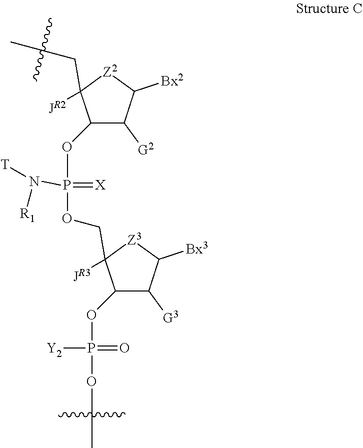

wherein:
each Bx is a heterocyclic base moiety;
X is selected from O or S;
each of Y₁ and Y₂ is independently selected from OH or SH;
each of Z² and Z³ are independently selected from —(CH₂)ₚ—X^{Z}—(CH₂)_{q}—, wherein p is 0 or 1, q is 0 or 1, and X^{Z} is O, S, or N(E₁);
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group;
R₅ is selected from OCH₃, OH, C₁-C₆ alkyl, and substituted C₁-C₆ alkyl;
   either J^{R}₂ and G² form a J^{R}₂ and G² bridge, or J^{R}₂ is H and G² is selected from H, OH, halogen or O—[C(R₆)(R₇)]ₙ—[(C═O)ₘ—X^{G}]ⱼ—R₈;
either J^{R3} and G³ form a J^{R3} and G³ bridge, or J^{R3} is H and G³ is selected from H, OH, halogen or O—[C(R₆)(R₇)]ₙ—[(C═O)ₘ—X^{G}]ⱼ—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

In certain embodiments, modified oligonucleotides comprise at least one region having Structure D:

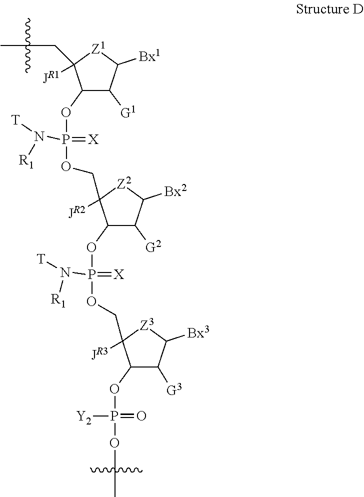

wherein:
each Bx is a heterocyclic base moiety;
X is selected from O or S;
each of Y₁ and Y₂ is independently selected from OH or SH;
each of Z² and Z³ are independently selected from —(CH₂)ₚ—X^{Z}—(CH₂)_{q}—, wherein p is 0 or 1, q is 0 or 1, and X^{Z} is O, S, or N(E₁);
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group;
R₅ is selected from OCH₃, OH, C₁-C₆ alkyl, and substituted C₁-C₆ alkyl;
either J^{R1} and G¹ form a J^{R1} to G¹ bridge, or J^{R1} is H and G¹ is selected from H, OH, halogen or O—[C(R₆)(R₇)]ⱼ—[(C═O)ₘ—X^{G}]ⱼ—R₈;
either J^{R2} and G² form a J^{R2} and G² bridge, or J^{R2} is H and G² is selected from H, OH, halogen or O—[C(R₆)(R₇)]ₙ—[(C═O)ₘ—X^{G}]—R₈;
either J^{R3} and G³ form a J^{R3} and G³ bridge, or J^{R3} is H and G³ is selected from H, OH, halogen or O—[C(R₆)(R₇)]ₙ—[(C═O)ₘ—X^{G}]—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

In certain embodiments, modified oligonucleotides comprise at least one region having Structure E:

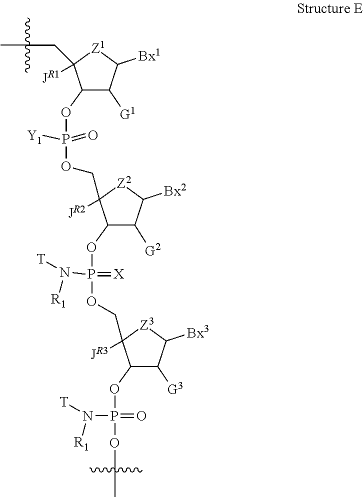

wherein:
each Bx is a heterocyclic base moiety;
X is selected from O or S;
each of Y₁ and Y₂ is independently selected from OH or SH;
each of Z² and Z³ are independently selected from —(CH₂)ₚ—X^{Z}—(CH₂)_{q}—, wherein p is 0 or 1, q is 0 or 1, and X^{Z} is O, S, or N(E₁);
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group;
R₅ is selected from OCH₃, OH, C₁-C₆ alkyl, and substituted C₁-C₆ alkyl;
either J^{R1} and G¹ form a J^{R1} to G¹ bridge, or J^{R1} is H and G¹ is selected from H, OH, halogen or O—[C(R₆)(R₇)]ₙ—[(C═O)ₘ—X^{G}]ⱼ—R₈;
either J^{R2} and G² form a J^{R2} and G² bridge, or J^{R2} is H and G² is selected from H, OH, halogen or O—[C(R₆)(R₇)]ₙ—[(C═O)ₘ—X^{G}]ⱼ—R₈;
either J^{R3} and G³ form a J^{R3} and G³ bridge, or J^{R3} is H and G³ is selected from H, OH, halogen or O—[C(R₆)(R₇)]ₙ—[(C═O)ₘ—X^{G}]ⱼ—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

In certain embodiments, modified oligonucleotides comprise a 5′-terminus having structure F:

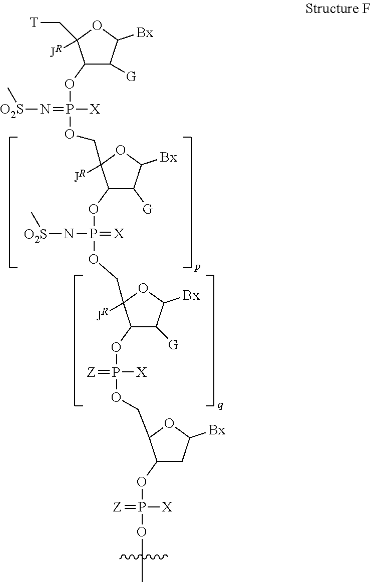

wherein:
p is from 0 to 6;
q is from 0 to 6;
T is OH, a stabilized phosphate group, or a conjugate group;
each Bx is an independently selected heterocyclic base moiety;
each X is independently selected from OH or SH;
each Z is independently selected from O, S, or NSO₂Me;
For each J^{R} and G of the same furanosyl sugar moiety, either J^{R} and G form a J^{R} to G bridge, or J^{R} is H and G is selected from OH, halogen or O—[C(R₆)(R₇)]ₙ—[(C═O)ₘ—X^{G}]ⱼ—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
   R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

In certain embodiments, modified oligonucleotides comprise a 3′-terminus having structure G:

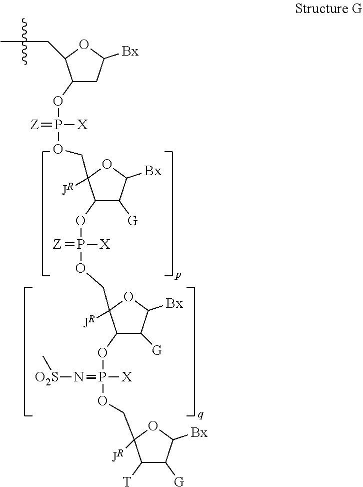

wherein:
p is from 0 to 6;
q is from 1 to 6;
T is OH or a conjugate group;
each Bx is an independently selected heterocyclic base moiety;
each X is independently selected from OH or SH;
each Z is independently selected from O, S, or NSO₂Me;
For each J^{R} and G of the same furanosyl sugar moiety, either J^{R} and G form a J^{R} to G bridge, or J^{R} is H and G is selected from OH, halogen or O—[C(R₆)(R₇)]ₙ—[(C═O)ₘ—X^{G}]ⱼ—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

In certain embodiments, modified oligonucleotides comprise a 5′-terminus having structure H:

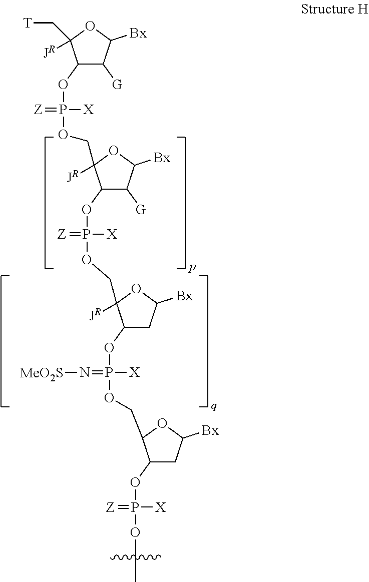

wherein:
p is from 0 to 5;
q is from 1 to 4;
T is OH, a stabilized phosphate group, or a conjugate group;
each Bx is an independently selected heterocyclic base moiety;
each X is independently selected from OH or SH;
each Z is independently selected from O, S, or NSO₂Me;
For each J^{R} and G of the same furanosyl sugar moiety, either J^{R} and G form a J^{R} to G bridge, or J^{R} is H and G is selected from OH, halogen or O—[C(R₆)(R₇)]ₙ[(C═O)ₘ—X^{G}]ⱼ—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

In certain embodiments, modified oligonucleotides comprise a 5′-terminus having structure I:

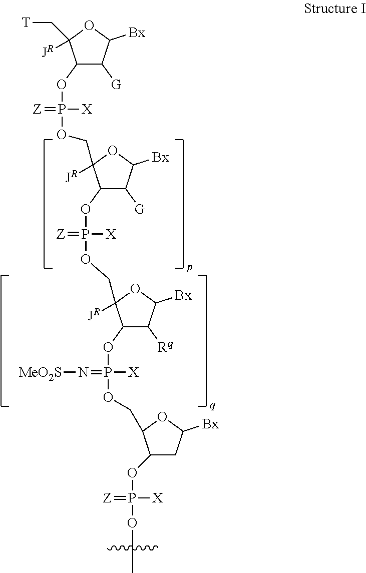

wherein:
p is from 0 to 5;
q is from 1 to 4;
T is OH, a stabilized phosphate group, or a conjugate group;
each Bx is an independently selected heterocyclic base moiety;
each X is independently selected from OH or SH;
each Z is independently selected from O, S, or NSO₂Me;
each R^{q} is H or exactly one R^{q} is OMe and the other R^{q} are H;
For each J^{R} and G of the same furanosyl sugar moiety, either J^{R} and G form a J^{R} to G bridge, or J^{R} is H and G is selected from OH, halogen or O—[C(R₆)(R₇)]ₙ-[(C═O)ₘ—X^{G}],R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

In certain embodiments, modified oligonucleotides comprise a 3′-terminus having structure J:

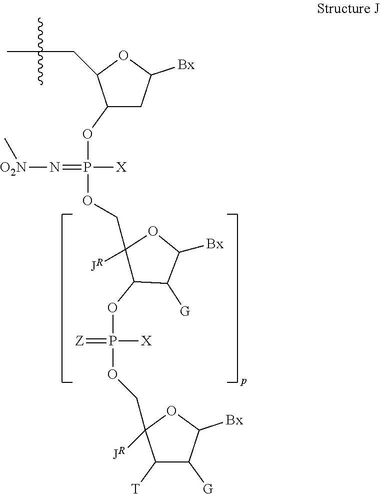

wherein:
p is from 0 to 6;
T is OH or a conjugate group;
each Bx is an independently selected heterocyclic base moiety;
each X is independently selected from OH or SH;
each Z is independently selected from O, S, or NSO₂Me;
For each J^{R} and G of the same furanosyl sugar moiety, either J^{R} and G form a J^{R} to G bridge, or J^{R} is H and G is selected from OH, halogen or O—[C(R₆)(R₇)]ₙ[(C═O)ₘ—X^{G}]ⱼ—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

In certain embodiments, modified oligonucleotides comprise a 5′-terminus having structure K:

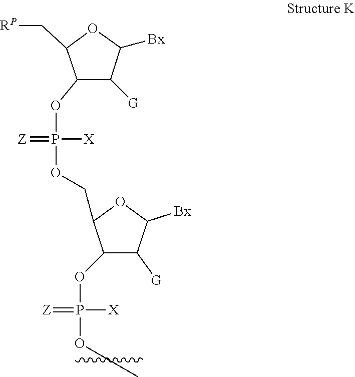

wherein:
R^{P} is a phosphate or stabilized phosphate group;
each Bx is an independently selected heterocyclic base moiety;
each X is independently selected from OH or SH;
each Z is selected from O, S, or NSO₂Me;
at least one Z is NSO₂Me;
each G is independently selected from OH, halogen or O—[C(R₆)(R₇)]ₙ[(C═O)ₘ—X^{G}]ⱼ—R₈;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

In certain embodiments, modified oligonucleotides comprise a 3′-terminus having structure L:

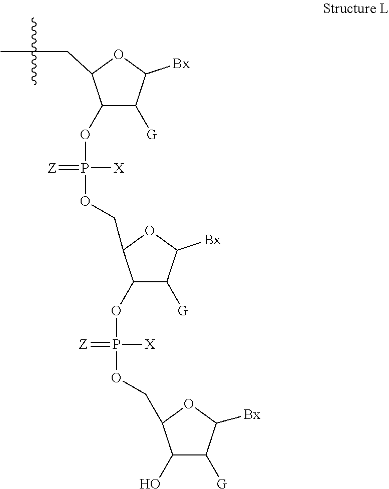

wherein:
   each Bx is an independently selected heterocyclic base moiety;
   each X is independently selected from OH or SH;
   each Z is selected from O, S, or NSO₂Me;
   at least one Z is NSO₂Me;
   each G is independently selected from OH, halogen or O—[C(R₆)(R₇)]˜[(C═O)ₘ—X^{G}]ⱼ—R₈; each R₆ and R₇ is, independently, H, halogen, C₁-C₆ alkyl or substituted C_{G}-C₆ alkyl;
   each X^{G} is O, S or N(E₁);
      R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
   E₁, E₂ and E₃ are each, independently, H, C,—C₆ alkyl or substituted C,—C₆ alkyl;
   n is from 1 to 6;
   m is 0 or 1;
   j is 0 or 1;
   each substituted group comprises one or more optionally protected substituent groups independently selected from halogen, OJ₁, N(J₁)(J₂), =NJ, SJ₁, N₃, CN, OC(=X₂)J₁, OC(=X₂)N(J₁)(J₂) and
   Q₂ is O, S or NJ₃;
   each J₁, J₂ and J₃ is, independently, H or C₁-C₆ alkyl.

In certain embodiments, modified oligonucleotides comprise at least one region having structure M:

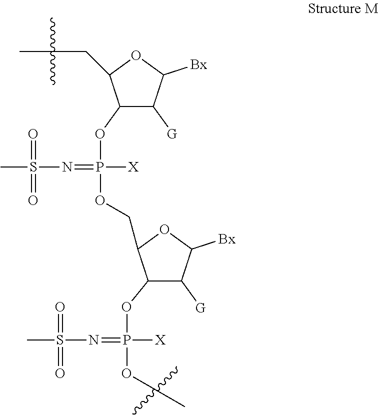

wherein:
   each Bx is an independently selected heterocyclic base moiety;
   each X is independently selected from OH or SH;
   each G is independently selected from OH, halogen or O—[C(R₆)(R₇)]ₙ[(C═O)ₘ—X^{G}]ⱼ—R₈;
   each R₆ and R₇ is, independently, H, halogen, C₁-C₆ alkyl or substituted C₁-C₆ alkyl;
   each X^{G} is O, S or N(E₁);
      R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
   E₁, E₂ and E₃ are each, independently, H, C₁-C₆ alkyl or substituted C₁-C₆ alkyl;
   n is from 1 to 6;
   m is 0 or 1;
   j is 0 or 1;
   each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
      Q₂ is O, S or NJ₃;
   each J₁, J₂ and J₃ is, independently, H or C₁-C₆ alkyl.
   In certain embodiments, modified oligonucleotides comprise a 5′-terminus having structure N:

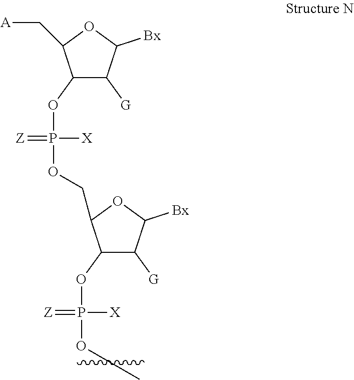

wherein:
A is selected from

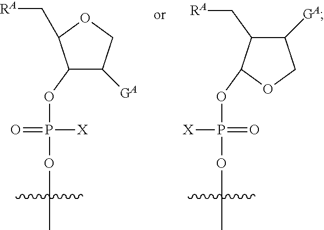

R^{A} is OH, OP(═O)OH, OP(═O)SH, or a stabilized phosphate group;
G^{A} is H, OH, OMe, MOE, or a halogen;
each Bx is an independently selected heterocyclic base moiety;
each X is independently selected from OH or SH;
each G is independently selected from OH, halogen or O—[C(R₆)(R₇)]ₙ—[(C═O)ₘ—X^{G}]ⱼ—R₈;
each R₆ and R₇ is, independently, H, halogen, C₁-C₆ alkyl or substituted C₁-C₆ alkyl;
each X^{G} is O, S or N(E₁);
   R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

In certain embodiments, modified oligonucleotides have a 3′-terminus having structure O:

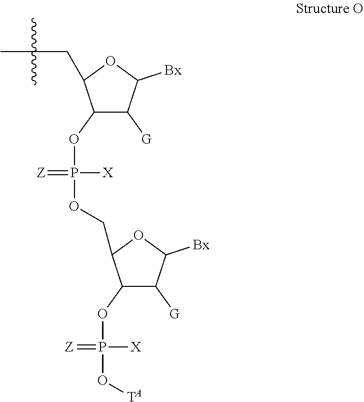

wherein:

T^{A} is selected from

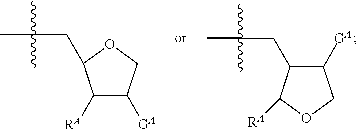

R^{A} G^{A} or R^{A} O
R^{A} is OH, OP(═O)OH, OP(═O)SH, or a stabilized phosphate group;
G^{A} is H, OH, OMe, MOE, or a halogen;
each Bx is an independently selected heterocyclic base moiety;
each X is independently selected from OH or SH;
each Z is selected from O, S, or NSO₂Me;
at least one Z is NSO₂Me;
each G is independently selected from OH, halogen or O—[C(R₆)(R₇)]ₙ[(C═O)ₘ—X^{G}]ⱼ—R₈;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

In certain embodiments, modified oligonucleotides comprise at least one region having the formula: (N_{g1})_{L1}(N_{g2})_{L2}(N_{g3})_{L3}, wherein each N_{g} is a nucleoside comprising furanosyl sugar moiety or a sugar surrogate and each L is an internucleoside linking group; wherein each of L₁, L₂, and L₃ is a phosphodiester internucleoside linking group, a phosphorothioate internucleoside linking group, or an internucleoside linking group of Formula XVII:

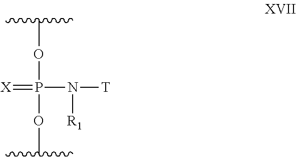

wherein each of L₁, and L₂ is a phosphodiester internucleoside linking group, a phosphorothioate internucleoside linking group, or an internucleoside linking group of Formula XVII:
wherein L₃ is absent or is a phosphodiester internucleoside linking group, a phosphorothioate internucleoside linking group, or an internucleoside linking group of Formula XVII;
wherein at least one of L₁, L₂, and L₃ an internucleoside linking group of Formula XVII; and at least one of L₁, L₂, and L₃ is a phosphorothioate or a phosphodiester internucleoside linking group,
wherein independently for each internucleoside linking group of the modified oligonucleotide having Formula XVII:
   X is selected from O or S;
   R₁ is selected from H, C₁-C₆ alkyl, and substituted C₁-C₆ alkyl; and
   T is selected from SO₂R₂, C(═O)R₃, and P(═O)R₄R₅, wherein:
   R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl, substituted C₁-C₆ alkynyl, and a conjugate group;
   R₃ is selected from an aryl, a substituted aryl, CH₃, N(CH₃)₂, OCH₃ and a conjugate;
   R₄ is selected from OCH₃, OH, C₁-C₆ alkyl, substituted C₁-C₆ alkyl and a conjugate; and
   R₅ is selected from OCH₃, OH, C₁-C₆ alkyl, and substituted C₁-C₆ alkyl.

In certain embodiments, the internucleoside linkages within the central region of a modified oligonucleotide are all modified with internucleoside linking groups having Formula VIII or Formula XVII. In certain embodiments, one internucleoside linkage within the central region of a modified oligonucleotide is an internucleoside linking group having Formula VIII or Formula XVII. In certain embodiments, two internucleoside linkages within the central region of a modified oligonucleotide are internucleoside linking groups having Formula VIII or Formula XVII. In certain embodiments, three internucleoside linkages within the central region of a modified oligonucleotide are internucleoside linking groups having Formula VIII or Formula XVII. In certain embodiments, four internucleoside linkages within the central region of a modified oligonucleotide are internucleoside linking groups having Formula VIII or Formula XVII. In certain embodiments, five internucleoside linkages within the central region of a modified oligonucleotide are internucleoside linking groups having Formula VIII or Formula XVII. In certain embodiments, six internucleoside linkages within the central region of a modified oligonucleotide are internucleoside linking groups having Formula VIII or Formula XVII. In certain embodiments, seven internucleoside linkages within the central region of a modified oligonucleotide are internucleoside linking groups having Formula VIII or Formula XVII. In certain embodiments, eight internucleoside linkages within the central region of a modified oligonucleotide are internucleoside linking groups having Formula VIII or Formula XVII. In certain embodiments, nine intemucleoside linkages within the central region of a modified oligonucleotide are internucleoside linking groups having Formula VIII or Formula XVII. In certain embodiments, ten intemucleoside linkages within the central region of a modified oligonucleotide are internucleoside linking groups having Formula VIII or Formula XVII. In certain embodiments, each internucleoside linkage within the central region of a modified oligonucleotide is an internucleoside linking groups having Formula VIII or Formula XVII.

In certain embodiments, the intemucleoside linking group linking the 2^{nd} and 3^{rd} nucleosides of the central region as counted from the 5′-end of the central region is an internucleoside linking group of Formula VIII or Formula XVII. In certain embodiments, the internucleoside linking group linking the 3^{rd} and 4^{th} nucleosides of the central region as counted from the 5′-end of the central region is an internucleoside linking group of Formula VIII or Formula XVII. In certain embodiments, the internucleoside linking group linking the 4^{th} and 5^{th} nucleosides of the central region as counted from the 5′-end of the central region is an internucleoside linking group of Formula VIII or Formula XVII.

In certain embodiments, the central region consists of 7-11 linked nucleosides, and has the formula: (N_{d1})_{L1}(N_{d2})_{L2}(N_{d3})_{L3}(N_{d4})_{L4}[(Nd)_{L5}]_{q};
wherein N_{d1}, N_{d2}, N_{d3}, N_{d4} are independently selected from among
a stereo-standard DNA nucleoside, a stereo-non-standard DNA nucleoside, or a 2′-substituted nucleoside; with the proviso that no more than one of N_{d1}, N_{d2}, N_{d3}, or N_{d4} is a 2′-substituted nucleoside;
each N_{d} is independently selected from among a stereo-standard DNA nucleoside and a stereo-non-standard DNA nucleoside;
q is from 3-8;
wherein each of L1, L₂, L₃, L₄, and each L₅ is an intemucleoside linkage;
wherein at least two of L₁, L₂, L₃, L₄ is an internucleoside linkage of Formula VIII or Formula XVII.

In certain embodiments, the oligonucleotide comprises at least one block of at least 3 consecutive internucleoside linking groups of Formula VIII or Formula XVII. In certain embodiments, the oligonucleotide comprises at least one block of at least 4 consecutive intemucleoside linking groups of Formula VIII or Formula XVII. In certain embodiments, the oligonucleotide comprises at least one block of at least 5 consecutive internucleoside linking groups of Formula VIII or Formula XVII. In certain embodiments, the oligonucleotide comprises at least one block of at least 5 consecutive internucleoside linking groups of Formula VIII or Formula XVII. In certain embodiments, the oligonucleotide comprises at least one block of at least 6 consecutive internucleoside linking groups of Formula VIII or Formula XVII. In certain embodiments, the oligonucleotide comprises at least one block of at least 7 consecutive internucleoside linking groups of Formula VIII or Formula XVII. In certain embodiments, the oligonucleotide comprises at least one block of at least 8 consecutive intemucleoside linking groups of Formula VIII or Formula XVII. In certain embodiments, the oligonucleotide comprises at least one block of at least 10 consecutive internucleoside linking groups of Formula VIII or Formula XVII. In certain embodiments, the oligonucleotide comprises at least one block of at least 12 consecutive internucleoside linking groups of Formula VIII or Formula XVII. In certain such embodiments, at least one such block is located at the 3′ end of the oligonucleotide. In certain such embodiments, at least one such block is located within 3 nucleosides of the 3′ end of the oligonucleotide. In certain such embodiments, at least one such block is located at the 5′ end of the oligonucleotide. In certain such embodiments, at least one such block is located within 3 nucleosides of the 5′ end of the oligonucleotide.

In certain such embodiments, some or all of the internucleoside linkages in the 5′-region and 3′-region are unmodified phosphate linkages. In certain embodiments, the terminal internucleoside linkages are modified. In certain embodiments, the internucleoside linkage motif comprises at least one phosphodiester internucleoside linkage in at least one of the 5′-region and the 3′-region, wherein the at least one phosphodiester linkage is not a terminal internucleoside linkage, and the remaining internucleoside linkages are internucleoside linking groups of Formula VIII or Formula XVII or phosphorothioate internucleoside linkages.

In certain embodiments, oligonucleotides comprise a region having an alternating internucleoside linkage motif. In certain embodiments, oligonucleotides comprise a region of uniformly modified internucleoside linkages. In certain such embodiments, the internucleoside linkages are internucleoside linking groups of Formula VIII or Formula XVII. In certain embodiments, each internucleoside linkage of the oligonucleotide is selected from phosphodiester, a phosphorothioate, and internucleoside linking group of Formula VIII or Formula XVII. In certain embodiments, each internucleoside linkage of the oligonucleotide is selected from phosphodiester or and an internucleoside linking groups of Formula VIII or Formula XVII. In certain embodiments, each internucleoside linkage of the oligonucleotide is selected from phosphorothioate and an internucleoside linking group of Formula VIII or Formula XVII.

In certain embodiments, each phosphorothioate internucleoside linkage is independently selected from a stereorandom phosphorothioate, a (Sp) phosphorothioate, and a (Rp) phosphorothioate. In certain embodiments, the internucleoside linkages within the central region of a modified oligonucleotide are all modified. In certain such embodiments, all of the phosphorothioate linkages are stereorandom. In certain embodiments, all of the phosphorothioate linkages in the 5′-region and 3′-region are (Sp) phosphorothioates, and the central region comprises at least one Sp, Sp, Rp motif. In certain embodiments, populations of modified oligonucleotides are enriched for modified oligonucleotides comprising such internucleoside linkage motifs.

In certain embodiments, the oligonucleotide comprises at least 6 phosphorothioate internucleoside linkages. In certain embodiments, the oligonucleotide comprises at least 8 phosphorothioate internucleoside linkages. In certain embodiments, the oligonucleotide comprises at least 10 phosphorothioate internucleoside linkages. In certain embodiments, the oligonucleotide comprises at least one block of at least 6 consecutive phosphorothioate internucleoside linkages. In certain embodiments, the oligonucleotide comprises at least one block of at least 8 consecutive phosphorothioate internucleoside linkages. In certain embodiments, the oligonucleotide comprises at least one block of at least 10 consecutive phosphorothioate internucleoside linkages. In certain embodiments, the oligonucleotide comprises at least block of at least one 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide.

In certain embodiments, oligonucleotides comprise one or more methylphosphonate linkages. In certain embodiments, modified oligonucleotides comprise a linkage motif comprising all phosphorothioate linkages except for one or two methylphosphonate linkages. In certain embodiments, one methylphosphonate linkage is in the central region of an oligonucleotide.

In certain embodiments, it is desirable to arrange the number of modified internucleoside linking groups having Formula VIII or Formula XVII, phosphorothioate internucleoside linkages, and phosphodiester internucleoside linkages to maintain nuclease resistance. In certain embodiments, it is desirable to arrange the number and position of modified internucleoside linking groups having Formula VIII or Formula XVII, phosphorothioate internucleoside linkages, and the number and position of phosphodiester internucleoside linkages to maintain nuclease resistance. In certain embodiments, the number of phosphorothioate internucleoside linkages may be decreased and the number of modified internucleoside linking groups having Formula VIII or Formula XVII and/or phosphodiester internucleoside linkages may be increased. In certain embodiments, the number of phosphorothioate internucleoside linkages may be decreased and the number of modified internucleoside linking groups having Formula VIII or Formula XVII and/or phosphodiester internucleoside linkages may be increased while still maintaining nuclease resistance. In certain embodiments it is desirable to decrease the number of phosphorothioate internucleoside linkages while retaining nuclease resistance. In certain embodiments it is desirable to increase the number of phosphodiester internucleoside linkages while retaining nuclease resistance.

In certain embodiments, the number of phosphodiester internucleoside linkages may be decreased by replacing phosphodiester internucleoside linkages with modified internucleoside linking groups having Formula VIII or Formula XVII. In certain embodiments, decreasing the number of phosphodiester internucleoside linkages and increasing the number of modified internucleoside linking groups having Formula VIII or Formula XVII increases the therapeutic index of a modified oligonucleotide or oligomeric compound. In certain embodiments, the number of phosphorothioate internucleoside linkages may be decreased by replacing phosphorothioate internucleoside linkages with modified internucleoside linking groups having Formula VIII or Formula XVII. In certain embodiments, decreasing the number of phosphorothioate internucleoside linkages and increasing the number of modified internucleoside linking groups having Formula VIII or Formula XVII increases the therapeutic index of a modified oligonucleotide or oligomeric compound.

In certain embodiments, a double-stranded antisense compound is a double-stranded RNAi compound comprising an RNAi antisense modified oligonucleotide and an RNAi sense modified oligonucleotide, wherein one or both of the RNAi antisense modified oligonucleotide and/or RNAi sense oligomeric compound have one or more modified internucleoside linking groups having Formula VIII or Formula XVII. In certain embodiments, the RNAi antisense modified oligonucleotide comprises at least two, at least three, at least four, at least five, or at least six modified internucleoside linking groups having Formula VIII or Formula XVII. In certain embodiments, the RNAi sense modified oligonucleotide comprises at least two, at least three, at least four, at least five, or at least six modified internucleoside linking groups having Formula VIII or Formula XVII.

In certain embodiments, the RNAi antisense modified oligonucleotide comprises exactly one modified internucleoside linking group having Formula VIII or Formula XVII. In certain embodiments, the RNAi antisense modified oligonucleotide comprises exactly two modified internucleoside linking groups having Formula VIII or Formula XVII. In certain embodiments, the RNAi antisense modified oligonucleotide comprises exactly three modified internucleoside linking groups having Formula VIII or Formula XVII. In certain embodiments, the RNAi antisense modified oligonucleotide comprises exactly four modified internucleoside linking groups having Formula VIII or Formula XVII. In certain embodiments, the RNAi antisense modified oligonucleotide comprises exactly five modified internucleoside linking groups having Formula VIII or Formula XVII. In certain embodiments, the RNAi antisense modified oligonucleotide comprises exactly six modified internucleoside linking groups having Formula VIII or Formula XVII. In certain embodiments, the RNAi antisense modified oligonucleotide comprises at least 6, at least 7, at least 8, or at least 9 modified internucleoside linking groups having Formula VIII or Formula XVII. In certain embodiments, each internucleoside linking group of the RNAi antisense modified oligonucleotide is a modified internucleoside linking groups having Formula VIII or Formula XVII.

In certain embodiments, the RNAi sense modified oligonucleotide comprises exactly one modified internucleoside linking group having Formula VIII or Formula XVII. In certain embodiments, the RNAi sense modified oligonucleotide comprises exactly two modified internucleoside linking groups having Formula VIII or Formula XVII. In certain embodiments, the RNAi sense modified oligonucleotide comprises exactly three modified internucleoside linking groups having Formula VIII or Formula XVII. In certain embodiments, the RNAi sense modified oligonucleotide comprises exactly four modified internucleoside linking groups having Formula VIII or Formula XVII. In certain embodiments, the RNAi sense modified oligonucleotide comprises exactly five modified internucleoside linking groups having Formula VIII or Formula XVII. In certain embodiments, the RNAi sense modified oligonucleotide comprises exactly six modified internucleoside linking groups having Formula VIII or Formula XVII. In certain embodiments, the RNAi sense modified oligonucleotide comprises at least 6, at least 7, at least 8, or at least 9 modified internucleoside linking groups having Formula VIII or Formula XVII. In certain embodiments, each internucleoside linking group of the RNAi sense modified oligonucleotide is a modified internucleoside linking groups having Formula VIII or Formula XVII.

In certain embodiments, at least one of the five 3′-most internucleoside linking groups of the RNAi antisense modified oligonucleotide is a modified internucleoside linking group having Formula VIII or Formula XVII. In certain embodiments, at least two of the five 3′-most internucleoside linking groups of the RNAi antisense modified oligonucleotide are modified internucleoside linking groups having Formula VIII or Formula XVII. In certain embodiments, at least one nucleoside of the seed region of the RNAi antisense modified oligonucleotide is a modified internucleoside linking group having Formula VIII or Formula XVII. In certain embodiments, at least one nucleoside within nucleosides 2 to 8 of the RNAi antisense modified oligonucleotide, counting from the 5′ end, is a modified internucleoside linking group having Formula VIII or Formula XVII.

In certain embodiments, the 5′-terminus of the RNAi antisense oligonucleotide has structure K:

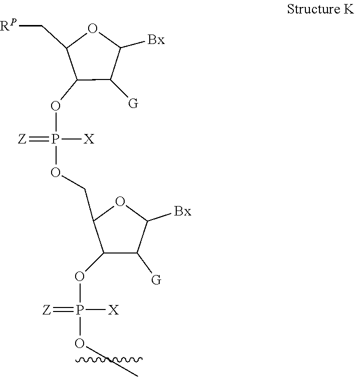

wherein:
R^{P} is a phosphate or stabilized phosphate group;
each Bx is an independently selected heterocyclic base moiety;
each X is independently selected from OH or SH;
each Z is selected from O, S, or NSO₂Me;
at least one Z is NSO₂Me;
each G is independently selected from OH, halogen or O—[C(R₆)(R₇)]ₙ[(C═O)ₘ—X^{G}]ⱼ—R₈;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

In certain embodiments, the 3′-terminus of the RNAi antisense oligonucleotide has structure L:

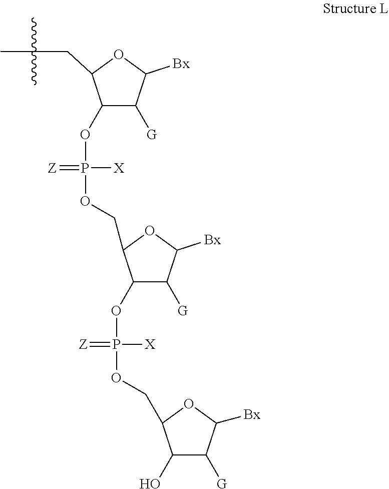

wherein:
   each Bx is an independently selected heterocyclic base moiety;
   each X is independently selected from OH or SH;
   each Z is selected from O, S, or NSO₂Me;
   at least one Z is NSO₂Me;
   each G is independently selected from OH, halogen or O—[C(R₆)(R₇)]ₙ[(C═O)ₘ—X^{G}]ⱼ—R₈;
   each R₆ and R₇ is, independently, H, halogen, C₁-C₆ alkyl or substituted C₁-C₆ alkyl;
   each X^{G} is O, S or N(E₁);
      R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
   E₁, E₂ and E₃ are each, independently, H, C₁-C₆ alkyl or substituted C₁-C₆ alkyl;
   n is from 1 to 6;
   m is 0 or 1;
   j is 0 or 1;
   each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
   Q₂ is O, S or NJ₃;
   each J₁, J₂ and J₃ is, independently, H or C₁-C₆ alkyl.

In certain embodiments, at least one region of the RNAi antisense oligonucleotide has structure M:

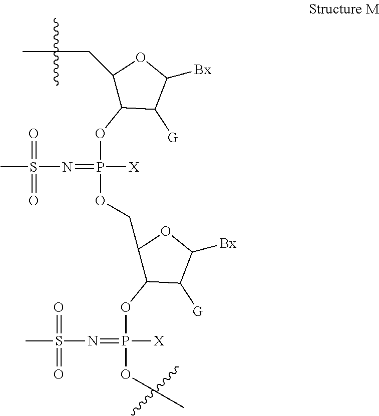

wherein:
   each Bx is an independently selected heterocyclic base moiety;
   each X is independently selected from OH or SH;
   each G is independently selected from OH, halogen or O—[C(R₆)(R₇)]ₙ—[(C═O)ₘ—X^{G}]ⱼ—R₈;
   each R₆ and R₇ is, independently, H, halogen, C₁-C₆ alkyl or substituted C₁-C₆ alkyl; each X^{G} is O, S or N(E₁);
      R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
   E₁, E₂ and E₃ are each, independently, H, C₁-C₆ alkyl or substituted C₁-C₆ alkyl;
   n is from 1 to 6;
   m is 0 or 1;
   j is 0 or 1;
      each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
   Q₂ is O, S or NJ3;
   each J₁, J₂ and J₃ is, independently, H or C₁-C₆ alkyl.

In certain embodiments, the 5′-terminus of the RNAi antisense oligonucleotide has structure N:

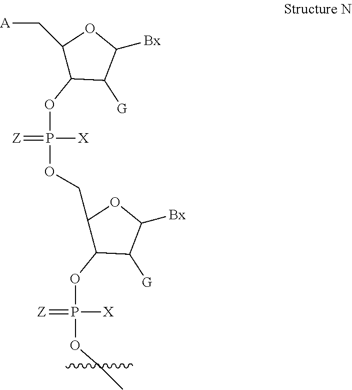

wherein:

A is selected from

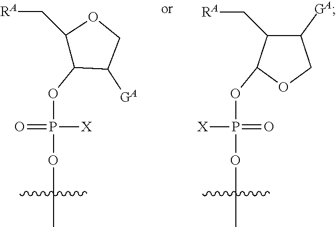

R^{A} is OH, OP(═O)OH, OP(═O)SH, or a stabilized phosphate group;
G^{A} is H, OH, OMe, MOE, or a halogen;
each Bx is an independently selected heterocyclic base moiety;
each X is independently selected from OH or SH;
each G is independently selected from OH, halogen or O—[C(R₆)(R₇)]ₙ—[(C═O)ₘ—X^{G}]ⱼ—R₈;
each R₆ and R₇ is, independently, H, halogen, C₁-C₆ alkyl or substituted C₁-C₆ alkyl;
each X^{G} is O, S or N(E₁);
   R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

In certain embodiments, the 3′-terminus of the RNAi antisense oligonucleotide has structure O:

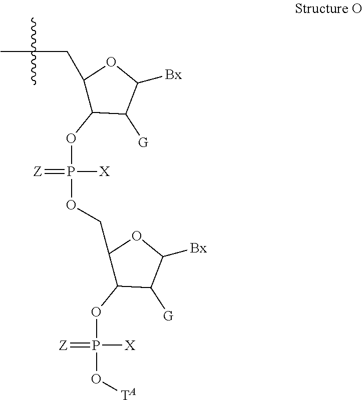

wherein:

T^{A} is selected from

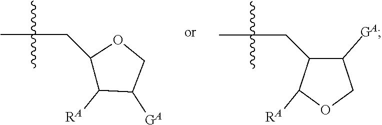

R^{A} is OH, OP(═O)OH, OP(═O)SH, or a stabilized phosphate group;
G^{A} is H, OH, OMe, MOE, or a halogen;
each Bx is an independently selected heterocyclic base moiety;
each X is independently selected from OH or SH;
each Z is selected from O, S, or NSO₂Me;
at least one Z is NSO₂Me;
each G is independently selected from OH, halogen or O—[C(R₆)(R₇)]ₙ[(C═O)ₘ—X^{G}]ⱼ—R₈;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

In certain embodiments, at least one of the first 5 internucleoside linkages from the 5′ end of the RNAi sense modified oligonucleotide is a modified internucleoside linking group having Formula VIII or Formula XVII. In certain embodiments, at least one of the five 3′-most internucleoside linking groups of the RNAi sense modified oligonucleotide is a modified internucleoside linking group having Formula VIII or Formula XVII. In certain embodiments, at least one of the first 5 internucleoside linkages from the 5′ end of the RNAi sense modified oligonucleotide and at least one of the five 3′-most internucleoside linking groups of the RNAi sense modified oligonucleotide is a modified internucleoside linking group having Formula VIII or Formula XVII. In certain embodiments, at least one nucleoside within nucleosides 2 to 8 of the RNAi sense modified oligonucleotide, counting from the 5′ end, is a modified internucleoside linking group having Formula VIII or Formula XVII.

In certain embodiments, the 5′-terminus of the RNAi sense oligonucleotide has structure K:

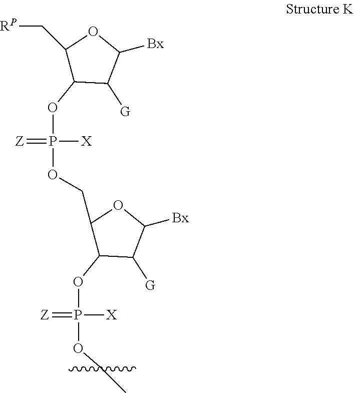

wherein:
R^{P} is a phosphate or stabilized phosphate group;
each Bx is an independently selected heterocyclic base moiety;
each X is independently selected from OH or SH;
each Z is selected from O, S, or NSO₂Me;
at least one Z is NSO₂Me;
each G is independently selected from OH, halogen or O—[C(R₆)(R₇)]ₙ[(C═O)ₘ—X^{G}]ⱼ—R₈;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

In certain embodiments, the 3′-terminus of the RNAi sense oligonucleotide has structure L:

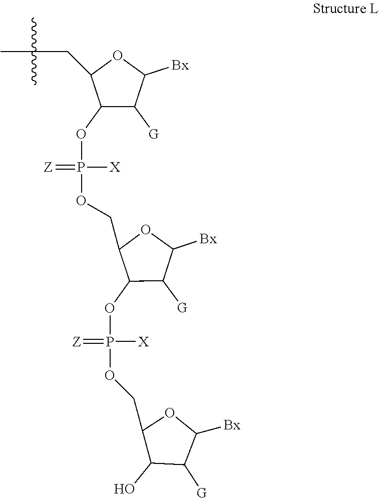

wherein:
   each Bx is an independently selected heterocyclic base moiety;
   each X is independently selected from OH or SH;
   each Z is selected from O, S, or NSO₂Me;
   at least one Z is NSO₂Me;
   each G is independently selected from OH, halogen or O—[C(R₆)(R₇)]ₙ[(C═O)ₘ—X^{G}]ⱼ—R₈;
   each R₆ and R₇ is, independently, H, halogen, C₁-C₆ alkyl or substituted C₁-C₆ alkyl;
   each X^{G} is O, S or N(E₁);
      R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
   E₁, E₂ and E₃ are each, independently, H, C₁-C₆ alkyl or substituted C₁-C₆ alkyl;
   n is from 1 to 6;
   m is 0 or 1;
   j is 0 or 1;
   each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
   Q₂ is O, S or NJ₃;
   each J₁, J₂ and J₃ is, independently, H or C₁-C₆ alkyl.

In certain embodiments, at least one region of the RNAi sense oligonucleotide has structure M:

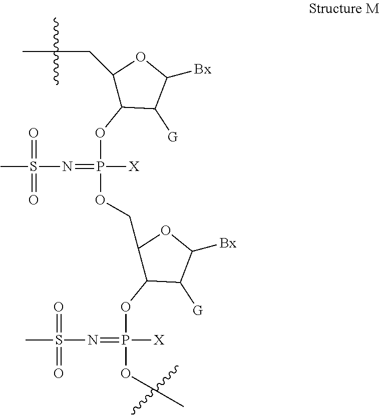

wherein:
   each Bx is an independently selected heterocyclic base moiety;
   each X is independently selected from OH or SH;
   each G is independently selected from OH, halogen or O—[C(R₆)(R₇)]˜-[(C═O)ₘ—X^{G}]ⱼ—R₈;
   each R₆ and R₇ is, independently, H, halogen, C₁-C₆ alkyl or substituted C₁-C₆ alkyl;
   each X^{G} is O, S or N(E₁);
      R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E2)(E3);
   E₁, E₂ and E₃ are each, independently, H, C₁-C₆ alkyl or substituted C₁-C₆ alkyl;
   n is from 1 to 6;
   m is 0 or 1;
   j is 0 or 1;
      each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
   Q₂ is O, S or NJ3;
   each J₁, J₂ and J₃ is, independently, H or C₁-C₆ alkyl.

In certain embodiments, the 5′-terminus of the RNAi sense oligonucleotide has structure N:

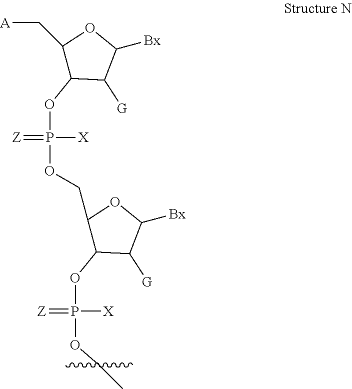

wherein:
A is selected from

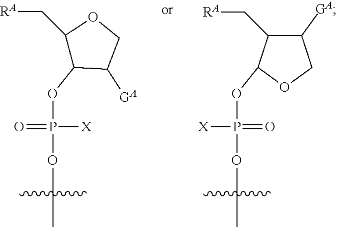

R^{A} is OH, OP(═O)OH, OP(═O)SH, or a stabilized phosphate group;
G^{A} is H, OH, OMe, MOE, or a halogen;
each Bx is an independently selected heterocyclic base moiety;
each X is independently selected from OH or SH;
each G is independently selected from OH, halogen or O—[C(R₆)(R₇)]ₙ[(C═O)ₘ—X^{G}]ⱼ—R₈;
each R₆ and R₇ is, independently, H, halogen, C₁-C₆ alkyl or substituted C₁-C₆ alkyl;
each X^{G} is O, S or N(E₁);
   R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

In certain embodiments, the 3′-terminus of the RNAi sense oligonucleotide has structure O:

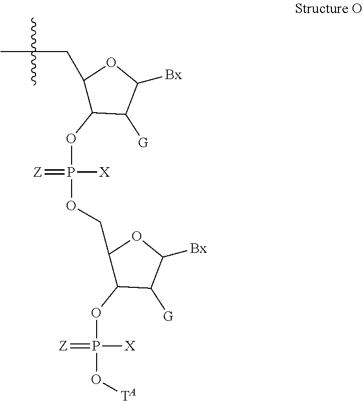

wherein:

T^{A} is selected from

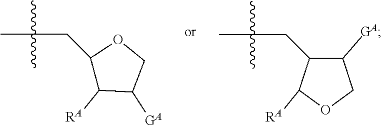

R^{A} G^{A} or R^{A} O
R^{A} is OH, OP(═O)OH, OP(═O)SH, or a stabilized phosphate group;
G^{A} is H, OH, OMe, MOE, or a halogen;
each Bx is an independently selected heterocyclic base moiety;
each X is independently selected from OH or SH;
each Z is selected from O, S, or NSO₂Me;
at least one Z is NSO₂Me;
each G is independently selected from OH, halogen or O—[C(R₆)(R₇)]ₙ[(C═O)ₘ—X^{G}]ⱼ—R₈;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

In certain embodiments, the 5′ terminus of the antisense oligonucleotide has structure P:

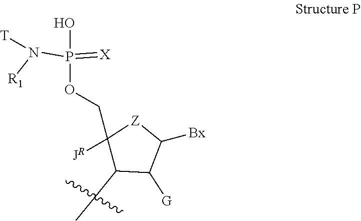

wherein:
each Bx is a heterocyclic base moiety;
X is selected from O or S;
Z is —(CH₂)ₚ—X^{Z}—(CH₂)_{q}—, wherein p is 0 or 1, q is 0 or 1, and X^{Z} is O, S, or N(E₁);
R₁ is selected from H, C₁-C₆ alkyl, and substituted C₁-C₆ alkyl; and
T is selected from SO₂R₂, C(═O)R₃, and P(═O)R₄R₅, wherein:
   R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group;
R₅ is selected from OCH₃, OH, C₁-C₆ alkyl, and substituted C₁-C₆ alkyl;
either J^{R} and G form a J^{R} to G bridge, or J^{R} is H and G is selected from H, OH, halogen or O—[C(R₆)(R₇)]—[(C═O)ₘ—X^{G}]ⱼ—R₈;
wherein each J^{R} to G bridge has a formula independently selected from —CH(CH₃)—O— or —(CH₂)ₖ—O—, wherein k is from 1 to 3;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=X₂)J₁, OC(=X₂)N(J₁)(J₂) and C(=Q₂)N(J₁)(J₂);
Q₂ is O, S or NJ₃;
each J₁, J₂ and J₃ is, independently, H or C₁-C₆ alkyl.

In certain embodiments, an oligomeric compound (including an oligomeric compound that is an antisense agent or a portion thereof) is a single-stranded RNAi compound or RNAi agent comprising an RNAi antisense modified oligonucleotide. In certain embodiments, the single-stranded RNAi compound or RNAi agent comprising an RNAi antisense modified oligonucleotide comprises at least two, at least three, at least four, at least five, or at least six modified internucleoside linking groups having Formula VIII or Formula XVII. In certain embodiments, the single-stranded RNAi compound or RNAi agent comprising an RNAi antisense modified oligonucleotide comprises exactly one modified internucleoside linking group having Formula VIII or Formula XVII. In certain embodiments, the single-stranded RNAi compound or RNAi agent comprising an RNAi antisense modified oligonucleotide comprises exactly two modified internucleoside linking groups having Formula VIII or Formula XVII. In certain embodiments, the single-stranded RNAi compound or RNAi agent comprising an RNAi antisense modified oligonucleotide comprises exactly three modified internucleoside linking groups having Formula VIII or Formula XVII. In certain embodiments, the single-stranded RNAi compound or RNAi agent comprising an RNAi antisense modified oligonucleotide comprises exactly four modified internucleoside linking groups having Formula VIII or Formula XVII. In certain embodiments, the single-stranded RNAi compound or RNAi agent comprising an RNAi antisense modified oligonucleotide comprises exactly five modified internucleoside linking groups having Formula VIII or Formula XVII. In certain embodiments, the single-stranded RNAi compound or RNAi agent comprising an RNAi antisense modified oligonucleotide comprises exactly six modified internucleoside linking groups having Formula VIII or Formula XVII. In certain embodiments, the single-stranded RNAi compound or RNAi agent comprising an RNAi antisense modified oligonucleotide comprises at least 6, at least 7, at least 8, or at least 9 modified internucleoside linking groups having Formula VIII or Formula XVII. In certain embodiments, each internucleoside linking group of the single-stranded RNAi compound or RNAi agent comprising an RNAi antisense modified oligonucleotide is a modified internucleoside linking groups having Formula VIII or Formula XVII.

In certain embodiments, at least one of the first 5 internucleoside linkages from the 5′ end of the single-stranded RNAi compound or RNAi agent comprising an RNAi antisense modified oligonucleotide is a modified internucleoside linking group having Formula VIII or Formula XVII. In certain embodiments, at least one of the five 3′-most internucleoside linking groups from the 3′ end of the single-stranded RNAi compound or RNAi agent comprising an RNAi antisense modified oligonucleotide is a modified internucleoside linking group having Formula VIII or Formula XVII. In certain embodiments, at least one nucleoside of the seed region of the single-stranded RNAi compound or RNAi agent comprising an RNAi antisense modified oligonucleotide is a modified internucleoside linking group having Formula VIII or Formula XVII. In certain embodiments, at least one nucleoside within nucleosides 2 to 8 of the single-stranded RNAi compound or RNAi agent comprising an RNAi antisense modified oligonucleotide, counting from the 5′ end, is a modified internucleoside linking group having Formula VIII or Formula XVII.

In certain embodiments, the 5′-terminus of the single-stranded RNAi compound or RNAi agent comprising an RNAi antisense modified oligonucleotide has a 5′-terminal group having formula XXI or XXII.

In certain embodiments, an oligomeric compound is a CRISPR compound. In certain embodiments, CRISPR compounds comprise a modified oligonucleotide that comprises a DNA recognition region and a tracrRNA recognition region. In certain embodiments, the DNA recognition region includes a seed region. In certain embodiments, CRISPR compounds have at least one modified internucleoside linking group having Formula VIII or Formula XVII. In certain embodiments, CRISPR compounds have at least two modified internucleoside linking groups having Formula VIII or Formula XVII. In certain embodiments, CRISPR compounds have at least three modified internucleoside linking groups having Formula VIII or Formula XVII. In certain embodiments, CRISPR compounds have at least four modified internucleoside linking groups having Formula VIII or Formula XVII. In certain embodiments, CRISPR compounds have at least five modified internucleoside linking groups having Formula VIII or Formula XVII. In certain embodiments, CRISPR compounds have at least six modified internucleoside linking groups having Formula VIII or Formula XVII. In certain embodiments, CRISPR compounds have at least 10 modified internucleoside linking groups having Formula VIII or Formula XVII. In certain embodiments, CRISPR compounds have at least 15 modified internucleoside linking groups having Formula VIII or Formula XVII. In certain embodiments, CRISPR compounds have at least 20 modified internucleoside linking groups having Formula VIII or Formula XVII. In certain embodiments, CRISPR compounds have at least 25 modified internucleoside linking groups having Formula VIII or Formula XVII. In certain embodiments, each internucleoside linking group of the CRISPR compound is a modified internucleoside linking group having Formula VIII or Formula XVII.

In certain embodiments, CRISPR compounds have exactly one modified internucleoside linking group having Formula VIII or Formula XVII. In certain embodiments, CRISPR compounds have exactly two modified internucleoside linking groups having Formula VIII or Formula XVII. In certain embodiments, CRISPR compounds have exactly three modified internucleoside linking groups having Formula VIII or Formula XVII. In certain embodiments, CRISPR compounds have exactly four modified internucleoside linking groups having Formula VIII or Formula XVII. In certain embodiments, CRISPR compounds have exactly five modified internucleoside linking groups having Formula VIII or Formula XVII. In certain embodiments, CRISPR compounds have exactly six modified internucleoside linking groups having Formula VIII or Formula XVII.

In certain embodiments, the DNA recognition portion of a CRISPR compound has at least one modified internucleoside linking group having Formula VIII or Formula XVII. In certain embodiments, the DNA recognition portion of a CRISPR compound has at least two modified internucleoside linking groups having Formula VIII or Formula XVII. In certain embodiments, the DNA recognition portion of a CRISPR compound has at least three modified internucleoside linking groups having Formula VIII or Formula XVII. In certain embodiments, the DNA recognition portion of a CRISPR compound has at least four modified internucleoside linking groups having Formula VIII or Formula XVII. In certain embodiments, the DNA recognition portion of a CRISPR compound has at least five modified internucleoside linking groups having Formula VIII or Formula XVII. In certain embodiments, the DNA recognition portion of a CRISPR compound has at least six modified internucleoside linking groups having Formula VIII or Formula XVII. In certain embodiments, the DNA recognition portion of a CRISPR compound has at least 10 modified internucleoside linking groups having Formula VIII or Formula XVII. In certain embodiments, each internucleoside linking group of the DNA recognition portion of a CRISPR compound is a modified internucleoside linking group having Formula VIII or Formula XVII.

In certain embodiments, the DNA recognition portion of a CRISPR compound has exactly one modified internucleoside linking group having Formula VIII or Formula XVII. In certain embodiments, the DNA recognition portion of a CRISPR compound has exactly two modified internucleoside linking groups having Formula VIII or Formula XVII. In certain embodiments, the DNA recognition portion of a CRISPR compound has exactly three modified internucleoside linking groups having Formula VIII or Formula XVII. In certain embodiments, the DNA recognition portion of a CRISPR compound has exactly four modified internucleoside linking groups having Formula VIII or Formula XVII. In certain embodiments, the DNA recognition portion of a CRISPR compound has exactly five modified internucleoside linking groups having Formula VIII or Formula XVII. In certain embodiments, the DNA recognition portion of a CRISPR compound has exactly six modified internucleoside linking groups having Formula VIII or Formula XVII. In certain embodiments, at least one internucleoside linking group of the of the seed region of the CRISPR oligonucleotide is a modified internucleoside linking group having Formula VIII or Formula XVII.

In certain embodiments, the tracrRNA recognition portion of a CRISPR compound has at least one modified internucleoside linking group having Formula VIII or Formula XVII. In certain embodiments, the tracrRNA recognition portion of a CRISPR compound has at least two modified internucleoside linking groups having Formula VIII or Formula XVII. In certain embodiments, the tracrRNA recognition portion of a CRISPR compound has at least three modified internucleoside linking groups having Formula VIII or Formula XVII. In certain embodiments, the tracrRNA recognition portion of a CRISPR compound has at least four modified internucleoside linking groups having Formula VIII or Formula XVII. In certain embodiments, the tracrRNA recognition portion of a CRISPR compound has at least five modified internucleoside linking groups having Formula VIII or Formula XVII. In certain embodiments, the tracrRNA recognition portion of a CRISPR compound has at least six modified internucleoside linking groups having Formula VIII or Formula XVII. In certain embodiments, the tracrRNA recognition portion of a CRISPR compound has at least 10 modified internucleoside linking groups having Formula VIII or Formula XVII. In certain embodiments, each internucleoside linking group of the tracrRNA recognition portion of a CRISPR compound is a modified internucleoside linking group having Formula VIII or Formula XVII.

In certain embodiments, the tracrRNA recognition portion of a CRISPR compound has exactly one modified internucleoside linking group having Formula VIII or Formula XVII. In certain embodiments, the tracrRNA recognition portion of a CRISPR compound has exactly two modified internucleoside linking groups having Formula VIII or Formula XVII. In certain embodiments, the tracrRNA recognition portion of a CRISPR compound has exactly three modified internucleoside linking groups having Formula VIII or Formula XVII. In certain embodiments, the tracrRNA recognition portion of a CRISPR compound has exactly four modified internucleoside linking groups having Formula VIII or Formula XVII. In certain embodiments, the tracrRNA recognition portion of a CRISPR compound has exactly five modified internucleoside linking groups having Formula VIII or Formula XVII. In certain embodiments, the tracrRNA recognition portion of a CRISPR compound has exactly six modified internucleoside linking groups having Formula VIII or Formula XVII.

In certain embodiments, an oligomeric compound is an artificial mRNA oligonucleotide. In certain embodiments, an oligomeric compound is an artificial mRNA oligonucleotide having a 5′UTR and a 3′UTR. In certain embodiments, the artificial mRNA oligonucleotide comprises more than 10, more than 20, more than 30, more than 40, more than 50, or more than 100 internucleoside linking groups having Formula VIII or Formula XVII.

In certain embodiments, the artificial mRNA oligonucleotide has exactly one modified internucleoside linking group having Formula VIII or Formula XVII. In certain embodiments, the artificial mRNA oligonucleotide has exactly two modified internucleoside linking groups having Formula VIII or Formula XVII. In certain embodiments, the artificial mRNA oligonucleotide has exactly three modified internucleoside linking groups having Formula VIII or Formula XVII. In certain embodiments, the artificial mRNA oligonucleotide has exactly four modified internucleoside linking groups having Formula VIII or Formula XVII. In certain embodiments, the artificial mRNA oligonucleotide has exactly five modified internucleoside linking groups having Formula VIII or Formula XVII. In certain embodiments, the artificial mRNA oligonucleotide has exactly six modified internucleoside linking groups having Formula VIII or Formula XVII.

In certain embodiments, the artificial mRNA oligonucleotide comprises exactly one modified internucleoside linking group having Formula VIII or Formula XVII. In certain embodiments, the artificial mRNA oligonucleotide comprises exactly two modified internucleoside linking groups having Formula VIII or Formula XVII. In certain embodiments, the artificial mRNA oligonucleotide comprises exactly three modified internucleoside linking groups having Formula VIII or Formula XVII. In certain embodiments, the artificial mRNA oligonucleotide comprises exactly four modified internucleoside linking groups having Formula VIII or Formula XVII. In certain embodiments, the artificial mRNA oligonucleotide comprises exactly five modified internucleoside linking groups having Formula VIII or Formula XVII. In certain embodiments, the artificial mRNA oligonucleotide comprises exactly 6, 7, 8, 9, 10, 11, 12, 13, 14, 15, 16, 17, 18, 19, 20, 21, or 22 modified internucleoside linking groups having Formula VIII or Formula XVII.

In certain embodiments, the artificial mRNA oligonucleotide comprises more than 10, more than 20, more than 30, more than 40, more than 50, or more than 100 modified internucleoside linking groups having Formula VIII or Formula XVII. In certain embodiments, at least one of the first 5 internucleoside linking groups from the 5′-end of the artificial mRNA oligonucleotide is a modified internucleoside linking group having Formula VIII or Formula XVII. In certain embodiments, at least one of the five 3′-most internucleoside linking groups of the artificial mRNA oligonucleotide is a modified internucleoside linking group having Formula VIII or Formula XVII. In certain embodiments, at least one internucleoside linking group of the 5′-UTR of the artificial mRNA oligonucleotide is a modified internucleoside linking group having Formula VIII or Formula XVII. In certain embodiments, at least one internucleoside linking group of the 3′-UTR of the artificial mRNA oligonucleotide is a modified internucleoside linking group having Formula VIII or Formula XVII. In certain embodiments, at least one internucleoside linking group of the coding region of the artificial mRNA oligonucleotide is a modified internucleoside linking group having Formula VIII or Formula XVII.

### II. Certain Modified Oligonucleotides

In certain embodiments, antisense agents, oligomeric compounds, and modified oligonucleotides described herein comprise or consist of modified oligonucleotides. In certain embodiments, the above modifications (sugar, nucleobase, internucleoside linkage) are incorporated into a modified oligonucleotide. In certain embodiments, modified oligonucleotides are characterized by their modifications, motifs, and overall lengths. In certain embodiments, such parameters are each independent of one another. Thus, unless otherwise indicated, each internucleoside linkage of a modified oligonucleotide may be modified or unmodified and may or may not follow the modification pattern of the sugar moieties. Likewise, such modified oligonucleotides may comprise one or more modified nucleobase independent of the pattern of the sugar modifications. Furthermore, in certain instances, a modified oligonucleotide is described by an overall length or range and by lengths or length ranges of two or more regions (e.g., a region of nucleosides having specified sugar modifications), in such circumstances it may be possible to select numbers for each range that result in an oligonucleotide having an overall length falling outside the specified range. In such circumstances, both elements must be satisfied. For example, in certain embodiments, a modified oligonucleotide consists of 15-20 linked nucleosides and has a sugar motif consisting of three regions or segments, A, B, and C, wherein region or segment A consists of 2-6 linked nucleosides having a specified sugar moiety, region or segment B consists of 6-10 linked nucleosides having a specified sugar moiety, and region or segment C consists of 2-6 linked nucleosides having a specified sugar moiety. Such embodiments do not include modified oligonucleotides where A and C each consist of 6 linked nucleosides and B consists of 10 linked nucleosides (even though those numbers of nucleosides are permitted within the requirements for A, B, and C) because the overall length of such oligonucleotide is 22, which exceeds the upper limit of 20 for the overall length of the modified oligonucleotide. Unless otherwise indicated, all modifications are independent of nucleobase sequence except that the modified nucleobase 5-methylcytosine is necessarily a “C” in an oligonucleotide sequence. In certain embodiments, when a DNA nucleoside or DNA-like nucleoside that comprises a T in a DNA sequence is replaced with a RNA-like nucleoside, the nucleobase T is replaced with the nucleobase U. Each of these compounds has an identical target RNA.

In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments oligonucleotides have a nucleobase sequence that is complementary to a second oligonucleotide or an identified reference nucleic acid, such as a target nucleic acid. In certain embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 70%, at least 80%, at least 90%, at least 95%, or 100% complementary to the second oligonucleotide or nucleic acid, such as a target nucleic acid.

### IV. Certain Conjugated Compounds

In certain embodiments, antisense agents, oligomeric compounds, and modified oligonucleotides described herein comprise or consist of a modified oligonucleotide that optionally comprises a conjugate group. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate moieties or terminal groups are attached at the 3′ and/or 5′-end of oligonucleotides. In certain such embodiments, conjugate moieties (or terminal groups) are attached at the 3′-end of oligonucleotides. In certain embodiments, conjugate moieties are attached near the 3′-end of oligonucleotides. In certain embodiments, conjugate moieties (or terminal groups) are attached at the 5′-end of oligonucleotides. In certain embodiments, conjugate moieties are attached near the 5′-end of oligonucleotides.

Examples of terminal groups include but are not limited to conjugate moieties, conjugate groups, capping groups, phosphate moieties, protecting groups, modified or unmodified nucleosides, and two or more nucleosides that are independently modified or unmodified.

### A. Certain Conjugate Groups and Conjugate Moieties

In certain embodiments, modified oligonucleotides comprise one or more conjugate moieties or conjugate groups. In certain embodiments, conjugate groups modify one or more properties of the molecule, including but not limited to pharmacodynamics, pharmacokinetics, stability, binding, absorption, tissue distribution, cellular distribution, cellular uptake, charge and clearance. In certain embodiments, conjugate moieties impart a new property on the molecule, e.g., fluorophores or reporter groups that enable detection of the molecule.

Certain conjugate groups have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N. Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a 25 phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic, a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, i, 923-937),a tocopherol group (Nishina et al., Molecular Therapy Nucleic Acids, 2015, 4, e220; doi:10.1038/mtna.2014.72 and Nishina et al., Molecular Therapy, 2008, 16, 734-740), or a GalNAc cluster (e.g., WO2014/179620).

a. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

b. Conjugate linkers

In certain embodiments, conjugate groups comprise a conjugate linker that attaches a conjugate moiety to the remainder of the modified oligonucleotide. In certain embodiments, a conjugate linker is a single chemical bond (i.e. conjugate moiety is attached to the remainder of the modified oligonucleotide via a conjugate linker through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to oligomeric compounds, such as the oligonucleotides provided herein. In general, a bifunctional linking moiety comprises at least two functional groups. One of the functional groups is selected to bind to a particular site on an oligomeric compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides.

In certain embodiments, conjugate linkers comprise no more than 1 linker-nucleoside. In certain embodiments, it is desirable for a conjugate group or conjugate moiety to be cleaved from the remainder of the oligonucleotide. For example, in certain circumstances oligomeric compounds (including oligomeric compounds that are antisense agents or portions thereof) or modified oligonucleotides comprising a particular conjugate moiety are better taken up by a particular cell type, but once the compound has been taken up, it is desirable that the conjugate group be cleaved to release an unconjugated oligonucleotide. Thus, certain conjugate moieties may comprise one or more cleavable moieties, typically within the conjugate linker. In certain embodiments, a cleavable moiety is a cleavable bond. In certain embodiments, a cleavable moiety is a group of atoms comprising at least one cleavable bond.

In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate or phosphodiester linkage between an oligonucleotide and a conjugate moiety or conjugate group.

In certain embodiments, a cleavable moiety comprises or consists of one or more linker-nucleosides. In certain such embodiments, one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is a nucleoside comprising a 2′-deoxyfuranosyl that is attached to either the 3′ or 5′-terminal nucleoside of an oligonucleotide by a phosphodiester internucleoside linkage and covalently attached to the remainder of the conjugate linker or conjugate moiety by a phosphodiester or phosphorothioate linkage. In certain such embodiments, the cleavable moiety is a nucleoside comprising a 2′-β-D-deoxyribosyl sugar moiety. In certain such embodiments, the cleavable moiety is 2′-deoxyadenosine.

c. Certain Cell-Targeting Conjugate Moieties

In certain embodiments, a conjugate group comprises a cell-targeting conjugate moiety. In certain embodiments, a conjugate group has the general formula:

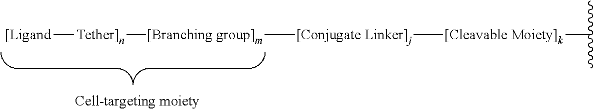

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, the cell-targeting moiety comprises a branching group comprising one or more groups selected from alkyl, amino, oxo, amide, disulfide, polyethylene glycol, ether, thioether and hydroxylamino groups. In certain embodiments, the branching group comprises a branched aliphatic group comprising groups selected from alkyl, amino, oxo, amide, disulfide, polyethylene glycol, ether, thioether and hydroxylamino groups. In certain such embodiments, the branched aliphatic group comprises groups selected from alkyl, amino, oxo, amide and ether groups. In certain such embodiments, the branched aliphatic group comprises groups selected from alkyl, amino and ether groups. In certain such embodiments, the branched aliphatic group comprises groups selected from alkyl and ether groups. In certain embodiments, the branching group comprises a mono or polycyclic ring system.

In certain embodiments, each tether of a cell-targeting moiety comprises one or more groups selected from alkyl, substituted alkyl, ether, thioether, disulfide, amino, oxo, amide, phosphodiester, and polyethylene glycol, in any combination. In certain embodiments, each tether is a linear aliphatic group comprising one or more groups selected from alkyl, ether, thioether, disulfide, amino, oxo, amide, and polyethylene glycol, in any combination. In certain embodiments, each tether is a linear aliphatic group comprising one or more groups selected from alkyl, phosphodiester, ether, amino, oxo, and amide, in any combination. In certain embodiments, each tether is a linear aliphatic group comprising one or more groups selected from alkyl, ether, amino, oxo, and amid, in any combination. In certain embodiments, each tether is a linear aliphatic group comprising one or more groups selected from alkyl, amino, and oxo, in any combination. In certain embodiments, each tether is a linear aliphatic group comprising one or more groups selected from alkyl and oxo, in any combination. In certain embodiments, each tether is a linear aliphatic group comprising one or more groups selected from alkyl and phosphodiester, in any combination. In certain embodiments, each tether comprises at least one phosphorus linking group or neutral linking group. In certain embodiments, each tether comprises a chain from about 6 to about 20 atoms in length. In certain embodiments, each tether comprises a chain from about 10 to about 18 atoms in length. In certain embodiments, each tether comprises about 10 atoms in chain length.

In certain embodiments, each ligand of a cell-targeting moiety has an affinity for at least one type of receptor on a target cell. In certain embodiments, each ligand has an affinity for at least one type of receptor on the surface of a mammalian lung cell.

In certain embodiments, each ligand of a cell-targeting moiety is a carbohydrate, carbohydrate derivative, modified carbohydrate, polysaccharide, modified polysaccharide, or polysaccharide derivative. In certain such embodiments, the conjugate group comprises a carbohydrate cluster (see, e.g., Maier et al., “Synthesis of Antisense Oligonucleotides Conjugated to a Multivalent Carbohydrate Cluster for Cellular Targeting,” Bioconjugate Chemistry, 2003, 14, 18-29, or Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, 47, 5798-5808, which are incorporated herein by reference in their entirety). In certain such embodiments, each ligand is an amino sugar or a thio sugar. For example, amino sugars may be selected from any number of compounds known in the art, such as sialic acid, α-D-galactosamine, β-muramic acid, 2-deoxy-2-methylamino-L-glucopyranose, 4,6-dideoxy-4-formamido-2,3-di-O-methyl-D-mannopyranose, 2-deoxy-2-sulfoamino-D-glucopyranose and N-sulfo-D-glucosamine, and N-glycoloyl-α-neuraminic acid. For example, thio sugars may be selected from 5-Thio-P-D-glucopyranose, methyl 2,3,4-tri-O-acetyl-1-thio-6-O-trityl-α-D-glucopyranoside, 4-thio-β-D-galactopyranose, and ethyl 3,4,6,7-tetra-O-acetyl-2-deoxy-1,5-dithio-α-D-gluco-heptopyranoside.

In certain embodiments, oligomeric compounds (including oligomeric compounds that are antisense agents or portions thereof) or modified oligonucleotides described herein comprise a conjugate group found in any of the following references: Lee, Carbohydr Res, 1978, 67, 509-514; Connolly et al., J Biol Chem, 1982, 257, 939-945; Pavia et al., Int J Pep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycobiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al., Bioorg Med Chem Lett, 2006, 16(19), 5132-5135; Maierhofer et al., Bioorg Med Chem, 2007, 15, 7661-7676; Khorev et al., Bioorg Med Chem, 2008, 16, 5216-5231; Lee et al., Bioorg Med Chem, 2011, 19, 2494-2500; Kornilova et al., Analyt Biochem, 2012, 425, 43-46; Pujol et al., Angew Chemie Int Ed Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromb Vasc Biol, 2006, 26, 169-175; van Rossenberg et al., Gene Ther, 2004, 11, 457-464; Sato et al., J. Am Chem Soc, 2004, 126, 14013-14022; Lee et al., J. Org Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 8,552,163; 6,908,903; 7,262,177; 5,994,517; 6,300,319; 8,106,022; 7,491,805; 7,491,805; 7,582,744; 8,137,695; 6,383,812; 6,525,031; 6,660,720; 7,723,509; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; Published U.S. patent Application Publications US2011/0097264; US2011/0097265; US2013/0004427; US2005/0164235; US2006/0148740; US2008/0281044; US2010/0240730; US2003/0119724; US2006/0183886; US2008/0206869; US2011/0269814; US2009/0286973; US2011/0207799; US2012/0136042; US2012/0165393; US2008/0281041; US2009/0203135; US2012/0035115; US2012/0095075; US2012/0101148; US2012/0128760; US2012/0157509; US2012/0230938; US2013/0109817; US2013/0121954; US2013/0178512; US2013/0236968; US2011/0123520; US2003/0077829; US2008/0108801; and US2009/0203132.

### Compositions and Methods for Formulating Pharmaceutical Compositions

Antisense agents, oligomeric compounds, and modified oligonucleotides described herein may be admixed with pharmaceutically acceptable active or inert substances for the preparation of pharmaceutical compositions. Compositions and methods for the formulation of pharmaceutical compositions are dependent upon a number of criteria, including, but not limited to, route of administration, extent of disease, or dose to be administered.

Certain embodiments provide pharmaceutical compositions comprising one or more oligomeric compounds (including oligomeric compounds that are antisense agents or portions thereof) or a salt thereof. In certain such embodiments, the pharmaceutical composition comprises a suitable pharmaceutically acceptable diluent or carrier. In certain embodiments, a pharmaceutical composition comprises a sterile saline solution and one or more oligomeric compound. In certain embodiments, such pharmaceutical composition consists of a sterile saline solution and one or more oligomeric compound. In certain embodiments, the sterile saline is pharmaceutical grade saline. In certain embodiments, a pharmaceutical composition comprises one or more oligomeric compound and sterile water. In certain embodiments, a pharmaceutical composition consists of one oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, a pharmaceutical composition consists of one or more oligomeric compound and sterile PBS. In certain embodiments, the sterile PBS is pharmaceutical grade PBS. Compositions and methods for the formulation of pharmaceutical compositions are dependent upon a number of criteria, including, but not limited to, route of administration, extent of disease, or dose to be administered.

An oligomeric compound described herein complementary to a target nucleic acid can be utilized in pharmaceutical compositions by combining the oligomeric compound with a suitable pharmaceutically acceptable diluent or carrier and/or additional components such that the pharmaceutical composition is suitable for injection. In certain embodiments, a pharmaceutically acceptable diluent is phosphate buffered saline. Accordingly, in one embodiment, employed in the methods described herein is a pharmaceutical composition comprising an oligomeric compound complementary to a target nucleic acid and a pharmaceutically acceptable diluent. In certain embodiments, the pharmaceutically acceptable diluent is phosphate buffered saline. In certain embodiments, the oligomeric compound comprises or consists of a modified oligonucleotide provided herein.

Pharmaceutical compositions comprising oligomeric compounds (including oligomeric compounds that are antisense agents or portions thereof) provided herein encompass any pharmaceutically acceptable salts, esters, or salts of such esters, or any other oligonucleotide which, upon administration to an animal, including a human, is capable of providing (directly or indirectly) the biologically active metabolite or residue thereof. In certain embodiments, the oligomeric compound comprises or consists of a modified oligonucleotide. Accordingly, for example, the disclosure is also drawn to pharmaceutically acceptable salts of compounds, prodrugs, pharmaceutically acceptable salts of such prodrugs, and other bioequivalents. Suitable pharmaceutically acceptable salts include, but are not limited to, sodium and potassium salts.

### Certain Mechanisms

In certain embodiments, oligomeric compounds (including oligomeric compounds that are antisense agents or portions thereof) described herein comprise or consist of modified oligonucleotides. In certain such embodiments, the oligomeric compounds described herein are capable of hybridizing to a target nucleic acid, resulting in at least one antisense activity. In certain embodiments, compounds described herein selectively affect one or more target nucleic acid. Such compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in a significant undesired antisense activity.

In certain antisense activities, hybridization of a compound described herein to a target nucleic acid results in recruitment of a protein that cleaves the target nucleic acid. For example, certain compounds described herein result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, compounds described herein are sufficiently “DNA-like” to elicit RNase H activity. Nucleosides that are sufficiently “DNA-like” to elicit RNase H activity are referred to as DNA mimics herein. Further, in certain embodiments, one or more non-DNA-like nucleoside in in the RNA:DNA duplex is tolerated.

In certain antisense activities, hybridization of an antisense agent, oligomeric compound, or modified oligonucleotide described herein to a target nucleic acid results in modulation of the splicing of a target pre-mRNA. For example, in certain embodiments, hybridization of a compound described herein will increase exclusion of an exon. For example, in certain embodiments, hybridization of a compound described herein will increase inclusion of an exon.

In certain antisense activities, antisense agents described herein or a portion of the antisense agent is loaded into an RNA-induced silencing complex (RISC), ultimately resulting in cleavage of the target nucleic acid. For example, certain compounds described herein result in cleavage of the target nucleic acid by Argonaute. Compounds that are loaded into RISC are RNAi compounds. RNAi compounds may be double-stranded (siRNA) or single-stranded (ssRNA).

In certain antisense activities, antisense agents, oligomeric compounds, or modified oligonucleotides described herein result in a CRISPR system cleaving a target DNA. In certain antisense activities, compounds described herein result in a CRISPR system editing a target DNA.

In certain antisense activities, hybridization of an antisense agent, oligomeric compound, or modified oligonucleotide described herein to a target nucleic acid results in disruption of secondary structural elements, such as stem-loops and hairpins. For example, in certain embodiments, hybridization of a compound described herein to a stem-loop that is part of a translation suppression element leads to an increase in protein expression.

In certain antisense activities, hybridization of an antisense agent, oligomeric compound, or modified oligonucleotide described herein to a target nucleic acid leads to no-go decay mediated mRNA degradation.

In certain antisense activities, hybridization of an antisense agent, oligomeric compound, or modified oligonucleotide described herein to a target nucleic acid leads to activation of nonsense-mediated decay mRNA degradation.

In certain embodiments, antisense agents, oligomeric compounds, or modified oligonucleotides described herein are artificial mRNA compounds, the nucleobase sequence of which encodes for a protein.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or animal.

### Certain RNAi Agents

In certain embodiments, oligomeric compounds described herein having one or more stinternucleoside linkages Formula VIII or Formula XVII are RNAi agents. In certain embodiments, internucleoside linkages having Formula VIII or Formula XVII can replace one or more phosphorothioate or phosphodiester internucleoside linkages in any RNAi motif. Certain RNAi motifs are described in, e.g., Freier, et al., WO2020/160163, incorporated by reference herein in its entirety; as well as, e.g., Rajeev, et al., WO2013/075035; Maier, et al., WO2016/028649; Theile, et al., WO2018/098328; Nair, et al., WO2019/217459; each of which is incorporated by reference herein.

### Target Nucleic Acids, Target Regions and Nucleotide Sequences

In certain embodiments, antisense agents, oligomeric compounds, or modified oligonucleotides described herein comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: an mRNA and a pre-mRNA, including intronic, exonic and untranslated regions. In certain embodiments, the target RNA is an mRNA. In certain embodiments, the target nucleic acid is a pre-mRNA. In certain embodiments, a pre-mRNA and corresponding mRNA are both target nucleic acids of a single compound. In certain such embodiments, the target region is entirely within an intron of a target pre-mRNA. In certain embodiments, the target region spans an intron/exon junction. In certain embodiments, the target region is at least 50% within an intron. In certain embodiments, the target nucleic acid is a microRNA. In certain embodiments, the target region is in the 5′ UTR of a gene. In certain embodiments, the target region is within a translation suppression element region of a target nucleic acid.

### Certain Compounds

Certain compounds described herein (e.g., antisense agents, oligomeric compounds, and modified oligonucleotides) have one or more asymmetric center and thus give rise to enantiomers, diastereomers, and other stereoisomeric configurations that may be defined, in terms of absolute stereochemistry, as (R) or (S), as α or β such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms. All tautomeric forms of the compounds provided herein are included unless otherwise indicated.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### Examples

The following examples are intended to illustrate certain aspects of the invention and are not intended to limit the invention in any way.

### Example 1: Synthesis of Modified Oligonucleotides with Mesyl Phosphoramidate Internucleoside Linkages

Modified oligonucleotides comprising a single mesyl phosphoramidate internucleoside linkage (Formula IX) were synthesized and tested. As shown in Table 1, each of the modified oligonucleotides has the same nucleobase sequence, GCATGTTCTCACATTA (SEQ ID NO: 5), which is 100% complementary to mouse CXCL12, GENBANK NT_039353.7 truncated from 69/430,515 to 69/445,350 (SEQ ID NO: 1), at position 6877 to 6892. The modified oligonucleotides are each 3-10-3 cEt gapmers with a sugar motif of: kkkddddddddddkkk (a 3-10-3 cEt motif) where “k” represents a cEt modified sugar moiety, and “d” represents a β-D-2′-deoxyribosyl sugar moiety. Each internucleoside linkage is either a phosphorothioate internucleoside linkage (“s”) or a mesyl phosphoramidate internucleoside linkage (“z”). Each of the compounds in Table 1 has exactly one mesyl phosphoramidate internucleoside linkage of formula IX.

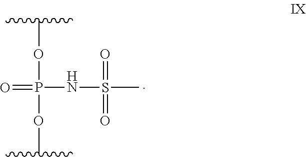 [SMILES: *OP(=O)(NS(C)(=O)=O)O*]

### Activity Assay

The modified oligonucleotides were tested for their ability to reduce target RNA in a series of experiments. Cultured mouse 3T3-L1 cells at a density of 20,000 cells per well were transfected using electroporation with modified oligonucleotides diluted to 20 μM, 7 μM, 2 μM, 0.7 μM, 0.3 μM, 0.1 μM, and 0.03 μM. After a treatment period of approximately 16 hours, CXCL12 RNA levels were measured using mouse primer-probe set RTS2605 (forward sequence CCAGAGCCAACGTCAAGCAT, SEQ ID NO: 2; reverse sequence: CAGCCGTGCAACAATCTGAA, SEQ ID NO: 3; probe sequence: TGAAAATCCTCAACACTCCAAACTGTGCC, SEQ ID NO: 4). CXCL12 RNA levels were normalized to total RNA content, as measured by RIBOGREEN®. Activity expressed as half maximal inhibitory concentration (IC50) was calculated using the log (inhibitor) vs response (three parameter) function in GraphPad Prism 7 and is presented in the table below.

TABLE 1
Design and activity of modified oligonucleotides having a
single mesyl phosphoramidate internucleoside linkage

| Compound | | IC50 | SEQ ID |
|---|---|---|---|
| Number | Chemistry Notation (5′-3′) | (nM) | NO. |
| 558807 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 84 | 5 |
| 1375403 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛT_{kz}Aₖ | 72 | 5 |
| 1375404 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}T_{kz}TₖₛAₖ | 61 | 5 |
| 1375405 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{dz}TₖₛTₖₛAₖ | 97 | 5 |
| 1375406 | Gₖₛ^{m}CₖₛAₖₛT_{ds}G_{ds}T_{ds}T_{ds}^{m}C_{ds}T_{ds}^{m}C_{ds}A_{ds}^{m}C_{dz}A_{ds}TₖₛTₖₛAₖ | 101 | 5 |
| 1375407 | Gₖₛ^{m}CₖₛAₖₛT_{ds}G_{ds}T_{ds}T_{ds}^{m}C_{ds}T_{ds}^{m}C_{ds}A_{dz}^{m}C_{ds}A_{ds}TₖₛTₖₛAₖ | 121 | 5 |
| 1375408 | Gₖₛ^{m}CₖₛAₖₛT_{ds}G_{ds}T_{ds}T_{ds}^{m}C_{ds}T_{ds}^{m}C_{dz}A_{ds}^{m}C_{ds}A_{ds}TₖₛTₖₛAₖ | 103 | 5 |
| 1375409 | Gₖₛ^{m}CₖₛAₖₛT_{ds}G_{ds}T_{ds}T_{ds}^{m}C_{ds}T_{dz}^{m}C_{ds}A_{ds}^{m}C_{ds}A_{ds}TₖₛTₖₛAₖ | 83 | 5 |
| 1375410 | Gₖₛ^{m}CₖₛAₖₛT_{ds}G_{ds}T_{ds}T_{ds}^{m}C_{dz}T_{ds}^{m}C_{ds}A_{ds}^{m}C_{ds}A_{ds}TₖₛTₖₛAₖ | 98 | 5 |
| 1375411 | Gₖₛ^{m}CₖₛAₖₛT_{ds}G_{ds}T_{ds}T_{dz}^{m}C_{ds}T_{ds}^{m}C_{ds}A_{ds}^{m}C_{ds}A_{ds}TₖₛTₖₛAₖ | 90 | 5 |
| 1375412 | Gₖₛ^{m}CₖₛAₖₛT_{ds}G_{ds}T_{dz}T_{ds}^{m}C_{ds}T_{ds}^{m}C_{ds}A_{ds}^{m}C_{ds}A_{ds}TₖₛTₖₛAₖ | 74 | 5 |
| 1375413 | Gₖₛ^{m}CₖₛAₖₛT_{ds}G_{dz}T_{ds}T_{ds}^{m}C_{ds}T_{ds}^{m}C_{ds}A_{ds}^{m}C_{ds}A_{ds}TₖₛTₖₛAₖ | 105 | 5 |
| 1375414 | Gₖₛ^{m}CₖₛAₖₛT_{dz}G_{ds}T_{ds}T_{ds}^{m}C_{ds}T_{ds}^{m}C_{ds}A_{ds}^{m}C_{ds}A_{ds}TₖₛTₖₛAₖ | 89 | 5 |
| 1375415 | Gₖₛ^{m}CₖₛA_{kz}T_{ds}G_{ds}T_{ds}T_{ds}^{m}C_{ds}T_{ds}^{m}C_{ds}A_{ds}^{m}C_{ds}A_{ds}TₖₛTₖₛAₖ | 43 | 5 |
| 1375416 | Gₖₛ^{m}C_{kz}AₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 84 | 5 |
| 1375417 | G_{kz}^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 76 | 5 |

A subscript “k” represents a cEt nucleoside, a subscript “d” represents a stereo-standard DNA nucleoside, a subscript “s” indicates phosphorothioate internucleoside linkage, a subscript “z” represents an internucleoside linkage of formula IX, which is a mesyl phosphoramidate linkage. A superscript “m” before a C represents a 5-methyl Cytosine

The phosphorothioate linkages were incorporated into the modified oligonucleotide using known processes. The phosphoramidate internucleoside linkages were incorporated into the modified oligonucleotides during synthesis using a Staudinger reaction with mesyl azide, a schematic of which is shown below:

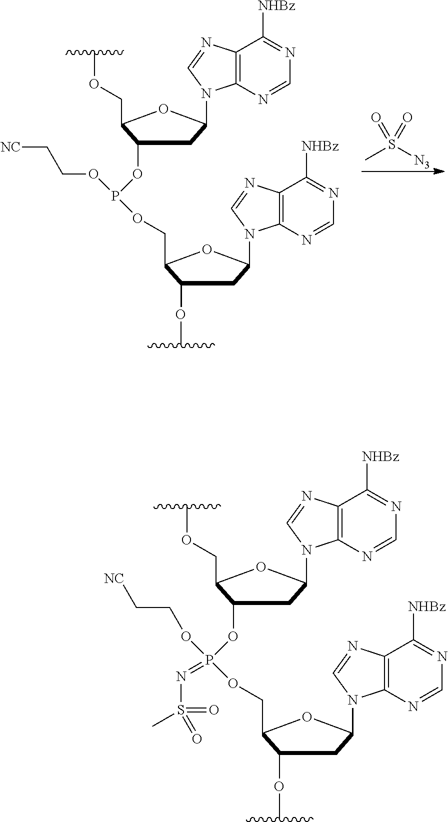

After the final nucleoside was added to the modified oligonucleotide, the modified oligonucleotide was deprotected and the intermediate linkage shown above was converted to the phosphoramidate internucleoside linkage shown below:

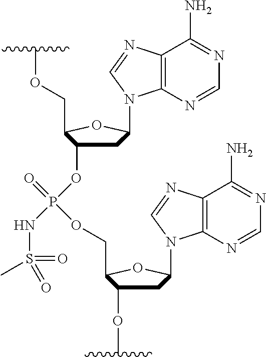

### Example 2: Design and Activity of Modified Oligonucleotides with Multiple Mesyl Phosphoramidate Internucleoside Linkages

Modified oligonucleotides comprising two consecutive (Table 2) or multiple (Table 3) mesyl phosphoramidate internucleoside linkages (Formula IX) were synthesized and tested. As shown in the tables below, each of the modified oligonucleotides has the same nucleobase sequence, GCATGTTCTCACATTA (SEQ ID NO: 5), which is 100% complementary to mouse CXCL12, GENBANK NT_039353.7 truncated from 69/430,515 to 69/445,350 (SEQ ID NO: 1), at position 6877 to 6892. The modified oligonucleotides are each 3-10-3 cEt gapmers with a sugar motif of: kkkddddddddddkkk (a 3-10-3 cEt motif) where “k” represents a cEt modified sugar moiety, and “d” represents a β-D-2′-deoxyribosyl sugar moiety. Each internucleoside linkage is either a phosphorothioate internucleoside linkage (“s”) or a mesyl phosphoramidate internucleoside linkage (“z”). Each of the compounds in Table 2 has two mesyl phosphoramidate internucleoside linkages of formula IX, and each compound in Table 3 has multiple mesyl phosphoramidate internucleoside linkages of formula IX.

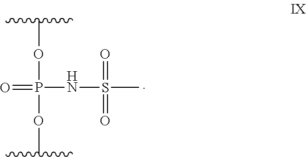 [SMILES: *OP(=O)(NS(C)(=O)=O)O*]

### Activity Assay

The modified oligonucleotides were tested for their ability to reduce target RNA in a series of experiments. Cultured mouse 3T3-L1 cells at a density of 20,000 cells per well were transfected using electroporation with modified oligonucleotides diluted to 20 μM, 7 μM, 2 μM, 0.7 μM, 0.3 μM, 0.1 μM, and 0.03 μM. After a treatment period of approximately 16 hours, CXCL12 RNA levels were measured using mouse primer-probe set RTS2605 (forward sequence CCAGAGCCAACGTCAAGCAT, SEQ ID NO: 2; reverse sequence: CAGCCGTGCAACAATCTGAA, SEQ ID NO: 3; probe sequence: TGAAAATCCTCAACACTCCAAACTGTGCC, SEQ ID NO: 4). CXCL12 RNA levels were normalized to total RNA content, as measured by RIBOGREEN® Activity expressed as half maximal inhibitory concentration (IC50) was calculated using the log (inhibitor) vs response (three parameter) function in GraphPad Prism 7 and is presented in the table below.

TABLE 2
Design and activity of modified oligonucleotides having
two consecutive mesyl phosphoramidate internucleoside linkages

| | | IC50 | IC50 | |
|---|---|---|---|---|
| Compound | | (nM) | (nM) | SEQ |
| Number | Chemistry Notation (5′-3′) | Study 1 | Study 2 | ID NO. |
| 558807 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 84 | 170 | 5 |
| 1375418 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}T_{kz}T_{kz}Aₖ | 16 | 117 | 5 |
| 1375419 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{dz}T_{kz}TₖₛAₖ | 23 | 105 | 5 |
| 1375420 | Gₖₛ^{m}CₖₛAₖₛT_{ds}G_{ds}T_{ds}T_{ds}^{m}C_{ds}T_{ds}^{m}C_{ds}A_{ds}^{m}C_{dz}A_{dz}TₖₛTₖₛAₖ | 41 | 138 | 5 |
| 1375421 | Gₖₛ^{m}CₖₛAₖₛT_{ds}G_{ds}T_{ds}T_{ds}^{m}C_{ds}T_{ds}^{m}C_{ds}A_{dz}^{m}C_{dz}A_{ds}TₖₛTₖₛAₖ | 37 | 83 | 5 |
| 1375422 | Gₖₛ^{m}CₖₛAₖₛT_{ds}G_{ds}T_{ds}T_{ds}^{m}C_{ds}T_{ds}^{m}C_{dz}A_{dz}^{m}C_{ds}A_{ds}TₖₛTₖₛAₖ | 61 | 83 | 5 |
| 1375423 | Gₖₛ^{m}CₖₛAₖₛT_{ds}G_{ds}T_{ds}T_{ds}^{m}C_{ds}T_{dz}^{m}C_{dz}A_{ds}^{m}C_{ds}A_{ds}TₖₛTₖₛAₖ | 40 | 123 | 5 |
| 1375424 | Gₖₛ^{m}CₖₛAₖₛT_{ds}G_{ds}T_{ds}T_{ds}^{m}C_{dz}T_{dz}^{m}C_{ds}A_{ds}^{m}C_{ds}A_{ds}TₖₛTₖₛAₖ | 38 | 146 | 5 |
| 1375425 | Gₖₛ^{m}CₖₛAₖₛT_{ds}G_{ds}T_{ds}T_{dz}^{m}C_{dz}T_{ds}^{m}C_{ds}A_{ds}^{m}C_{ds}A_{ds}TₖₛTₖₛAₖ | 33 | 71 | 5 |
| 1375426 | Gₖₛ^{m}CₖₛAₖₛT_{ds}G_{ds}T_{dz}T_{dz}^{m}C_{ds}T_{ds}^{m}C_{ds}A_{ds}^{m}C_{ds}A_{ds}TₖₛTₖₛAₖ | 32 | 100 | 5 |
| 1375427 | Gₖₛ^{m}CₖₛAₖₛT_{ds}G_{dz}T_{dz}T_{ds}^{m}C_{ds}T_{ds}^{m}C_{ds}A_{ds}^{m}C_{ds}A_{ds}TₖₛTₖₛAₖ | 28 | 87 | 5 |
| 1375428 | Gₖₛ^{m}CₖₛAₖₛT_{dz}G_{dz}T_{ds}T_{ds}^{m}C_{ds}T_{ds}^{m}C_{ds}A_{ds}^{m}C_{ds}A_{ds}TₖₛTₖₛAₖ | 33 | 99 | 5 |
| 1375429 | Gₖₛ^{m}CₖₛA_{kz}T_{dz}G_{ds}T_{ds}T_{ds}^{m}C_{ds}T_{ds}^{m}C_{ds}A_{ds}^{m}C_{ds}A_{ds}TₖₛTₖₛAₖ | 39 | 83 | 5 |
| 1375430 | Gₖₛ^{m}C_{kz}A_{kz}T_{ds}G_{ds}T_{ds}T_{ds}^{m}C_{ds}T_{ds}^{m}C_{ds}A_{ds}^{m}C_{ds}A_{ds}TₖₛTₖₛAₖ | 21 | 83 | 5 |
| 1375431 | G_{kz}^{m}C_{kz}AₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 27 | 108 | 5 |

A subscript “k” represents a cEt nucleoside, a subscript “d” represents a stereo-standard DNA nucleoside, a subscript “s” indicates phosphorothioate internucleoside linkage, a subscript “z” represents an internucleoside linkage of formula IX, which is a mesyl phosphoramidate linkage. A superscript “m” before a C represents a 5-methyl Cytosine

TABLE 3
Design and activity of modified oligonucleotides containing
multiple mesyl phosphoramidate internucleoside linkages

| Compound | | IC50 | SEQ ID |
|---|---|---|---|
| Number | Chemistry Notation (5′-3′) | (nM) | NO. |
| 558807 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 61 | 5 |
| 1375432 | G_{kz}^{m}C_{kz}A_{kz}T_{dz}G_{dz}T_{dz}T_{dz}^{m}C_{dz}T_{dz}^{m}C_{dz}A_{dz}^{m}C_{dz}A_{dz}T_{kz}T_{kz}Aₖ | 232 | 5 |
| 1386094 | Gₖₛ^{m}CₖₛA_{kz}T_{dz}G_{dz}T_{dz}T_{dz}^{m}C_{dz}T_{dz}^{m}C_{dz}A_{dz}^{m}C_{dz}A_{dz}TₖₛTₖₛAₖ | 810 | 5 |
| 1378793 | Gₖₛ^{m}CₖₛA_{kz}T_{dz}G_{dz}T_{dz}T_{dz}^{m}C_{dz}T_{dz}^{m}C_{dz}A_{dz}^{m}C_{dz}A_{ds}TₖₛTₖₛAₖ | 646 | 5 |
| 1386355 | Gₖₛ^{m}CₖₛAₖₛT_{dz}G_{dz}T_{dz}T_{dz}^{m}C_{dz}T_{dz}^{m}C_{dz}A_{dz}^{m}C_{dz}A_{ds}TₖₛTₖₛAₖ | 1158 | 5 |
| 1378794 | G_{kz}^{m}C_{kz}AₖₛT_{ds}G_{ds}T_{ds}T_{ds}^{m}C_{ds}T_{ds}^{m}C_{ds}A_{ds}^{m}C_{ds}A_{dz}T_{kz}T_{kz}Aₖ | 83 | 5 |

A subscript “k” represents a cEt nucleoside, a subscript “d” represents a stereostandard DNA nucleoside, a subscript “s” indicates a phosphorothioate internucleoside linkage, a subscript “z” represents an internucleoside linkage of formula IX, which is a mesyl phosphoramidate linkage. A superscript “m” before a C represents a 5-methyl Cytosine.

### Example 3: Caspase Activity of Modified Oligonucleotides in In Vitro Caspase Activation Assays

The modified oligonucleotides were tested for their ability to mediate caspase activity in a series of experiments. Cultured mouse HEPA1-6 cells at a density of 20,000 cells per well were transfected using electroporation with modified oligonucleotides diluted to 20 μM. After a treatment period of approximately 16 hours, caspase-3 and caspase-7 activation was measured using the Caspase-Glo® 3/7 Assay System (G8090, Promega). Results are shown in the tables below. Increased levels of caspase activation correlate with apoptotic cell death and cytotoxicity.

In some cases, the caspase activation mediated by the modified oligonucleotide was confirmed in an additional study. In such cases, the table shows % mock values for both studies 1 and 2 in separate columns.

TABLE 4
In vitro Caspase activation by modified oligonucleotides containing
a single mesyl phosphoramidate internucleoside linkage

| | Caspase |
|---|---|
| Compound | Activation |
| No. | (% Mock) |
| 558807 | 2727 |
| 1375403 | 2508 |
| 1375404 | 3089 |
| 1375405 | 2660 |
| 1375406 | 2427 |
| 1375407 | 2938 |
| 1375408 | 2546 |
| 1375409 | 2966 |
| 1375410 | 2744 |
| 1375411 | 2571 |
| 1375412 | 1808 |
| 1375413 | 2639 |
| 1375414 | 3028 |
| 1375415 | 2733 |
| 1375416 | 2714 |
| 1375417 | 2627 |

TABLE 5
In vitro Caspase activation by modified oligonucleotides containing
two consecutive mesyl phosphoramidate internucleoside linkages

| | Caspase | Caspase |
|---|---|---|
| | Activation | Activation |
| Compound | (% Mock) | (% Mock) |
| No. | Study 1 | Study 2 |
| 558807 | 738 | 848 |
| 1375418 | 821 | 955 |
| 1375419 | 1163 | 1192 |
| 1375420 | 683 | 623 |
| 1375421 | 814 | 749 |
| 1375422 | 662 | 767 |
| 1375423 | 900 | 1165 |
| 1375424 | 1305 | 1212 |
| 1375425 | 606 | 609 |
| 1375426 | 297 | 292 |
| 1375427 | 204 | 193 |
| 1375428 | 398 | 550 |
| 1375429 | 1032 | 728 |
| 1375430 | 1157 | 1271 |
| 1375431 | 869 | 844 |

TABLE 6
In vitro Caspase activation by modified oligonucleotides containing
multiple mesyl phosphoramidate internucleoside linkages

| | Caspase |
|---|---|
| Compound | Activation |
| No. | (% Mock) |
| 558807 | 2290 |
| 1375432 | 161 |
| 1386094 | 142 |
| 1378793 | 165 |
| 1386355 | 141 |
| 1378794 | 2500 |

### Example 4: Stability of Modified Oligonucleotides Containing Mesyl Phosphoramidate Internucleoside Linkages

The thermal stability (Tm) of duplexes of each of modified oligonucleotides described in the examples above with a complementary RNA 20-mer having the sequence GAUAAUGUGAGAACAUGCCU (SEQ ID NO: 6) was tested. Each modified oligonucleotide was separately hybridized with the complementary RNA strand to form a duplex. Once the duplex was formed, it was slowly heated and the melting temperature was measured using a spectrophotometer and the hyperchromicity method. Results are provided in the table below. This example demonstrates that mesyl phosphoramidate internucleoside linkages can be incorporated into modified oligonucleotides without destabilizing the interaction between the modified oligonucleotide and its complement.

TABLE 7
Tm of modified oligonucleotides complementary to CXCL12

| Compound No. | Tm (° C.) |
|---|---|
| 558807 | 63.5 |
| 1375403 | 63.8 |
| 1375404 | 63.9 |
| 1375405 | 63.5 |
| 1375406 | 63.7 |
| 1375407 | 63.6 |
| 1375408 | 63.9 |
| 1375409 | 64.2 |
| 1375410 | 63.9 |
| 1375411 | 64.0 |
| 1375412 | 63.7 |
| 1375413 | 63.6 |
| 1375414 | 63.3 |
| 1375415 | 63.7 |
| 1375416 | 63.7 |
| 1375417 | 63.3 |
| 1375418 | 63.3 |
| 1375419 | 63.7 |
| 1375420 | 63.4 |
| 1375421 | 63.6 |
| 1375422 | 63.8 |
| 1375423 | 64.2 |
| 1375424 | 64.2 |
| 1375425 | 64.4 |
| 1375426 | 64.4 |
| 1375427 | 63.9 |
| 1375428 | 63.1 |
| 1375429 | 63.4 |
| 1375430 | 63.8 |
| 1375431 | 64.1 |
| 1375432 | 63.7 |
| 1386094 | 64.6 |
| 1378793 | 63.9 |
| 1386355 | 63.3 |
| 1378794 | 63.7 |

### Example 5: Design and Synthesis of Modified Internucleoside Linkages

Additional modified internucleoside linkages described herein may be prepared via a Staudinger reaction similar to the reaction in Example 1, but where a substituted azide is used in place of the mesyl azide in Example 1.

For example, during the synthesis of a modified oligonucleotide, reaction of substituted azide (1) below with a 2-cyanoethyl phosphite internucleoside linkage will form the modified oligonucleotide intermediate shown below:

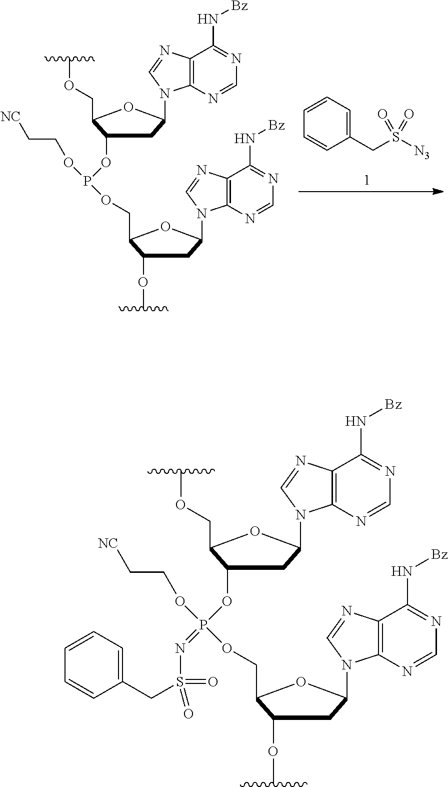

Upon deprotection and purification of the modified oligonucleotide, the modified internucleoside linkage intermediate above becomes the modified internucleoside linkage below:

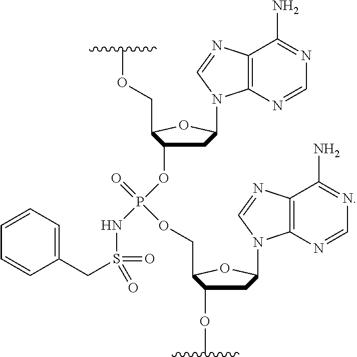

Alternatively, during the synthesis of a modified oligonucleotide, reaction of substituted azide (2) below with a 2-cyanoethyl phosphite internucleoside linkage will form the modified oligonucleotide intermediate shown below:

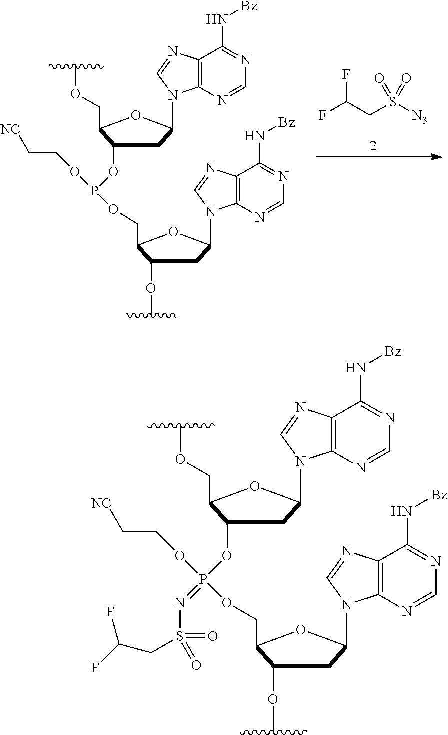

Upon deprotection and purification of the modified oligonucleotide, the modified internucleoside linkage intermediate above becomes the modified internucleoside linkage below:

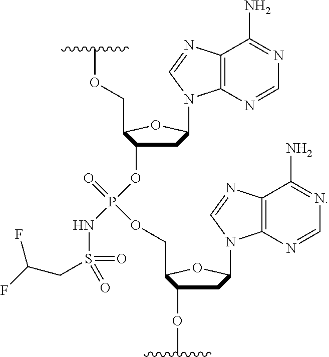

Alternatively, during the synthesis of a modified oligonucleotide, reaction of substituted azide (3) below with a 2-cyanoethyl phosphite internucleoside linkage will form the modified oligonucleotide intermediate shown below:

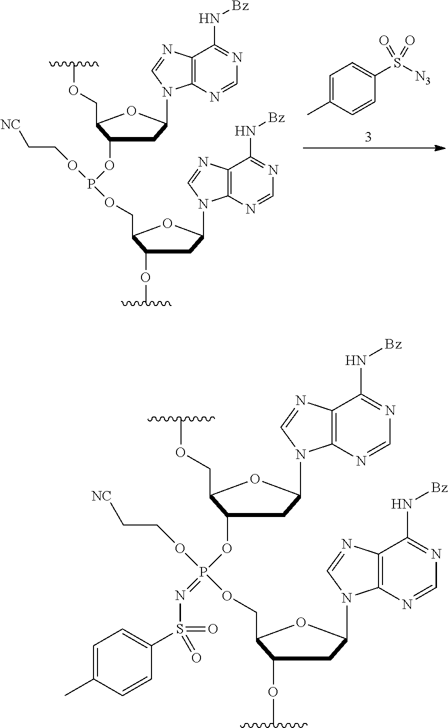

Upon deprotection and purification of the modified oligonucleotide, the modified intemucleoside linkage intermediate above becomes the modified internucleoside linkage below:

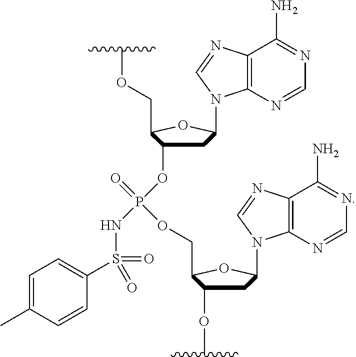

Alternatively, during the synthesis of a modified oligonucleotide, reaction of substituted azide (4) below with a 2-cyanoethyl phosphite internucleoside linkage will form the modified oligonucleotide intermediate shown below:

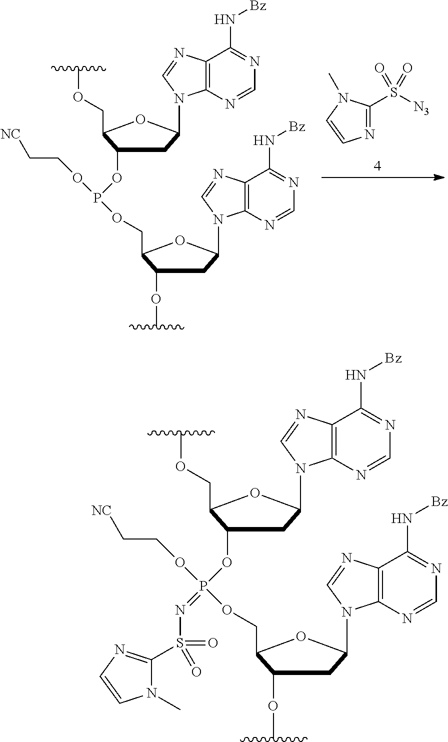

Upon deprotection and purification of the modified oligonucleotide, the modified internucleoside linkage intermediate above becomes the modified internucleoside linkage below:

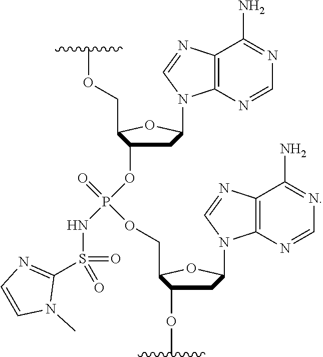

Alternatively, during the synthesis of a modified oligonucleotide, reaction of substituted azide (5) below with a 2-cyanoethyl phosphite internucleoside linkage will form the modified oligonucleotide intermediate shown below:

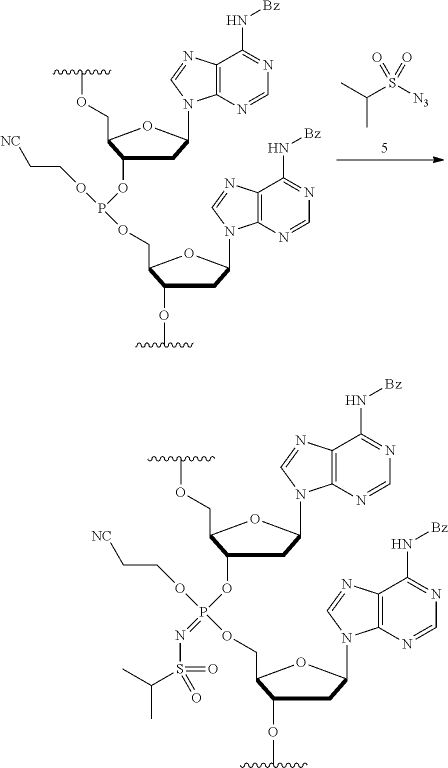

Upon deprotection and purification of the modified oligonucleotide, the modified internucleoside linkage intermediate above becomes the modified internucleoside linkage below:

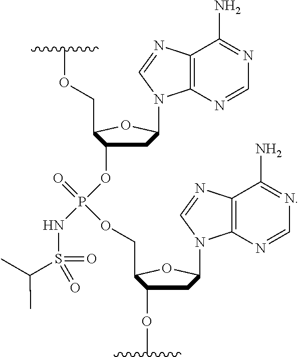

Alternatively, during the synthesis of a modified oligonucleotide, reaction of substituted azide (6) below with a 2-cyanoethyl phosphite internucleoside linkage will form the modified oligonucleotide intermediate shown below:

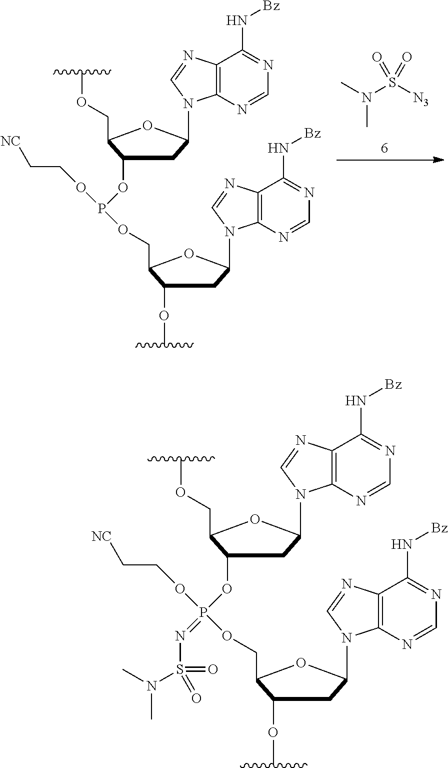

Upon deprotection and purification of the modified oligonucleotide, the modified internucleoside linkage intermediate above becomes the modified internucleoside linkage below:

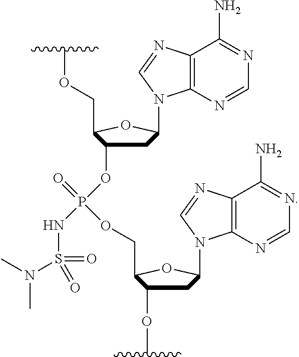

Alternatively, during the synthesis of a modified oligonucleotide, reaction of substituted azide (7) below with a 2-cyanoethyl phosphite internucleoside linkage will form the modified oligonucleotide intermediate shown below:

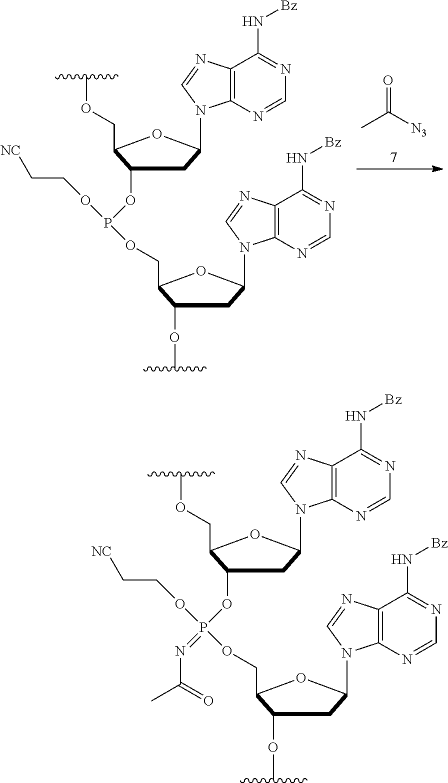

Upon deprotection and purification of the modified oligonucleotide, the modified internucleoside linkage intermediate above becomes the modified internucleoside linkage below:

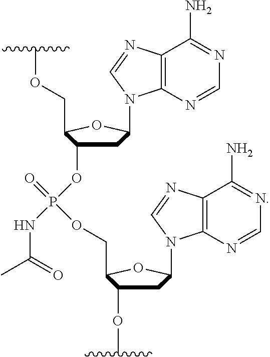

Alternatively, during the synthesis of a modified oligonucleotide, reaction of substituted azide (8) below with a 2-cyanoethyl phosphite internucleoside linkage will form the modified oligonucleotide intermediate shown below:

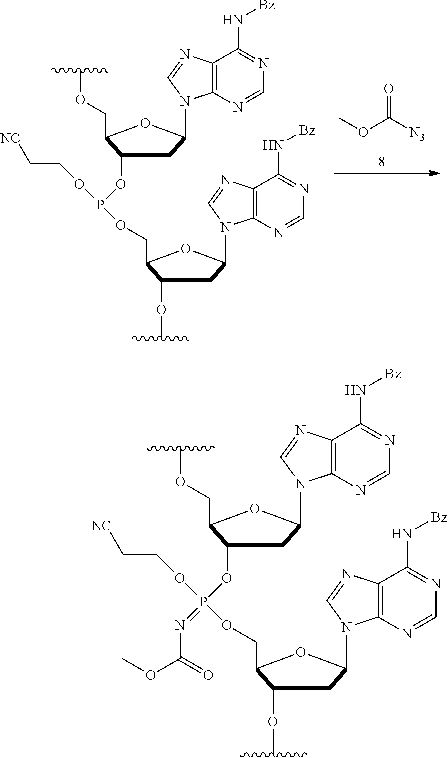

Upon deprotection and purification of the modified oligonucleotide, the modified internucleoside linkage intermediate above becomes the modified internucleoside linkage below:

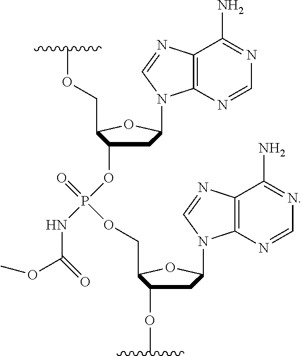

Alternatively, during the synthesis of a modified oligonucleotide, reaction of substituted azide (9) below with a 2-cyanoethyl phosphite internucleoside linkage will form the modified oligonucleotide intermediate shown below:

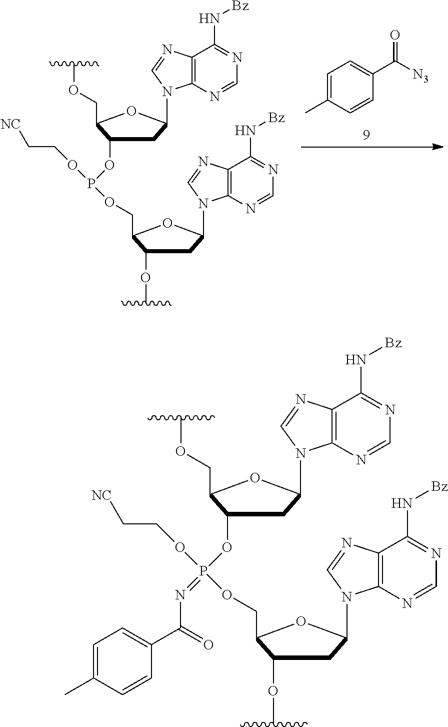

Upon deprotection and purification of the modified oligonucleotide, the modified internucleoside linkage intermediate above becomes the modified internucleoside linkage below:

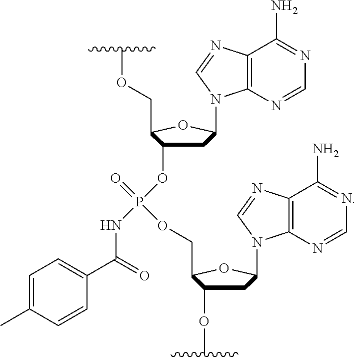

Alternatively, during the synthesis of a modified oligonucleotide, reaction of substituted azide (10) below with a 2-cyanoethyl phosphite internucleoside linkage will form the modified oligonucleotide intermediate shown below:

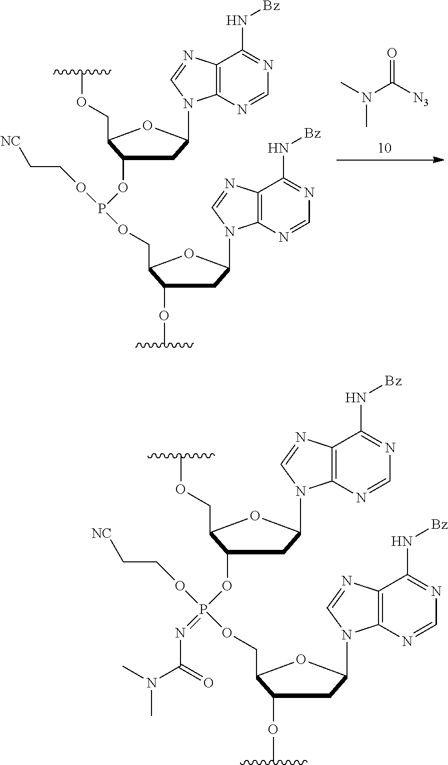

Upon deprotection and purification of the modified oligonucleotide, the modified internucleoside linkage intermediate above becomes the modified internucleoside linkage below:

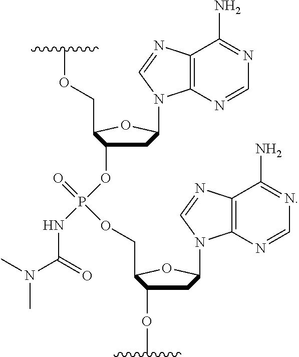

Alternatively, during the synthesis of a modified oligonucleotide, reaction of substituted azide (11) below with a 2-cyanoethyl phosphite internucleoside linkage will form the modified oligonucleotide intermediate shown below:

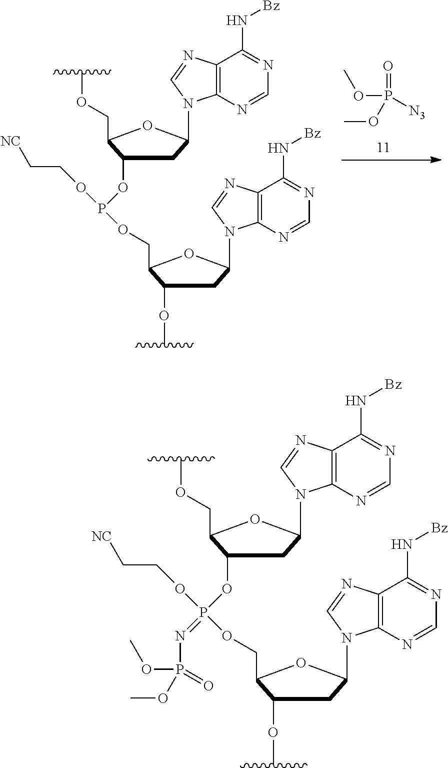

Upon deprotection and purification of the modified oligonucleotide, the modified internucleoside linkage intermediate above becomes the modified internucleoside linkage below:

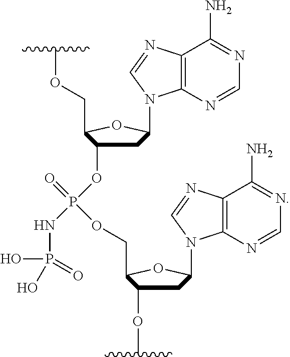

Alternatively, during the synthesis of a modified oligonucleotide, reaction of substituted azide (12) below with a 2-cyanoethyl phosphite internucleoside linkage will form the modified oligonucleotide intermediate shown below:

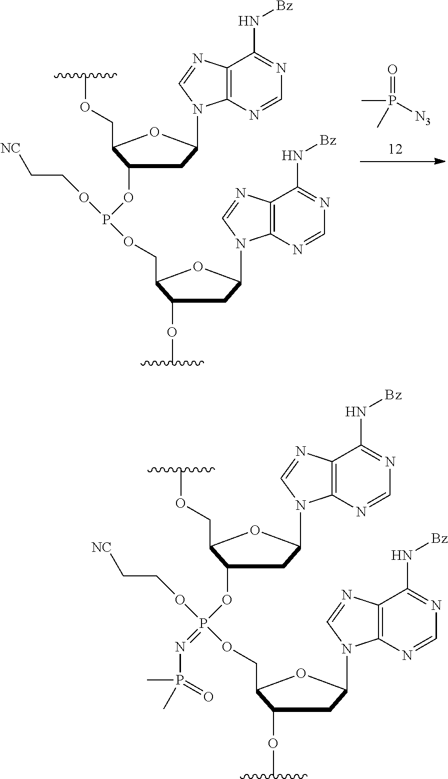

Upon deprotection and purification of the modified oligonucleotide, the modified internucleoside linkage intermediate above becomes the modified internucleoside linkage below:

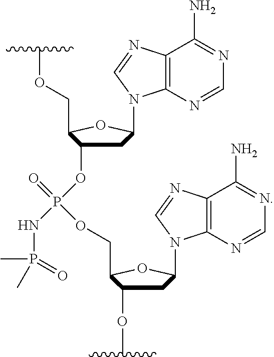

Alternatively, during the synthesis of a modified oligonucleotide, reaction of substituted azide (13) below with a 2-cyanoethyl phosphite internucleoside linkage will form the modified oligonucleotide intermediate shown below:

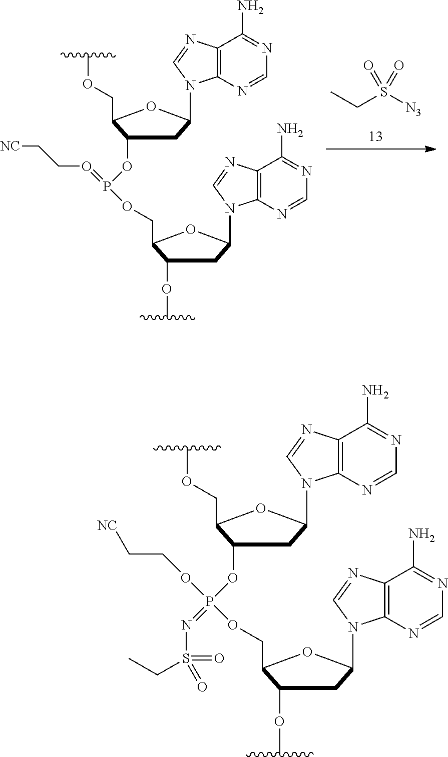

Upon deprotection and purification of the modified oligonucleotide, the modified internucleoside linkage intermediate above becomes the modified internucleoside linkage below:

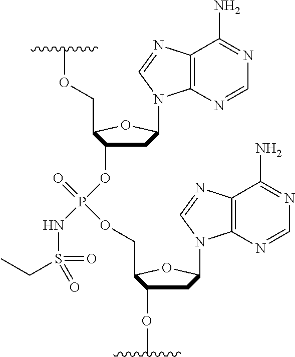

Alternatively, during the synthesis of a modified oligonucleotide, reaction of substituted azide (14) below with a 2-cyanoethyl phosphite internucleoside linkage will form the modified oligonucleotide intermediate shown below:

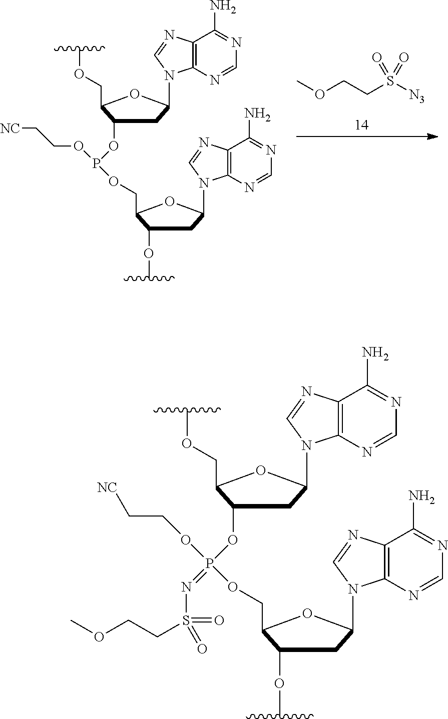

Upon deprotection and purification of the modified oligonucleotide, the modified internucleoside linkage intermediate above becomes the modified internucleoside linkage below:

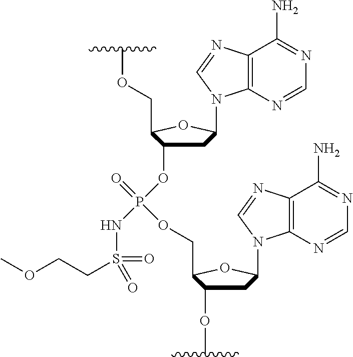

Additional substituted azides are known and readily available or easily synthesized.

### Example 6: Activity and Tolerability of Modified Oligonucleotides with Mesyl Phosphoramidate Internucleoside Linkages In Vivo

For the in vivo activity and tolerability study in the tables below, 3 BALB/C mice per group were administered modified oligonucleotide by subcutaneous injection and sacrificed after 72 hours. Compound 558807 was dosed at 1.8, 5.5, 16.7, or 50 mg/kg, while other modified oligonucleotides were dosed at 1.8, 5.5, 16.7, 50, or 150 mg/kg.

Tissue were collected and mRNA was isolated and levels of CXCL 12 in both liver and kidney samples were measured by RT-qPCR with primer probe set RTS2605 as described above. Levels of P21 were analyzed using primer probe set Mm04207341_ml (ThermoFisher) in liver and kidney and levels of Tnfrsf10b were analyzed using primer probe set Mm00457866_ml (ThermoFisher) in liver. Elevated P21 or Tnfrsf10b indicates toxicity. Plasma ALT was measured. Elevations in ALT are associated with liver toxicity.

Expression levels were normalized with Ribogreen® and are presented relative to levels in mice treated with PBS. In addition to compounds containing a mesyl phosphoramidate internucleoside linkage, Compound No. 936053 was tested. This compound has the sequence GCATGTTCTCACATTA (SEQ ID NO: 5) and a sugar motif of kkk-d-m-dddddddd-kkk, wherein each “k” represents a cEt nucleoside, each “d” represents a stereo-standard DNA nucleoside, and “m” represents a 2′-OMe nucleoside. Compound No. 936053 was described in WO2019/157531, and is included as a comparator compound as it has reduced toxicity relative to 558807 as well as reduced potency in vivo. Note that at least some of the observed potency of 558807 is “false”; that is, the RNA reduction observed is not specific to RNAse H mediated reduction of CXCL12 RNA, but rather, is related to global reductions in RNA due to cellular toxicity. Therefore, Compound No. 936503 represents a better comparator compound for determining the relative in vivo potency of compounds comprising mesyl phosphoramidate internucleoside linkages.

TABLE 8
In Vivo Activity and Toxicity of modified oligonucleotides
complementary to CXCL12

| | | in vivo | in vivo | | |
|---|---|---|---|---|---|
| | position of | CXCL12 | CXCL12 | ALT | |
| | modifications | ED50 | ED50 | @50 | ALT @ |
| Compound | in the gap (5′ | liver | kidney | mg/kg | 150 mg/kg |
| ID | to 3′) | (mg/kg) | (mg/kg) | (IU/L) | (IU/L) |
| 558807 | n/a | 1.0 | 31 | 7666 @ 50 mg/kg | |
| 936053 | nucleoside 2 | 5.5 | 38 | 23 | 29 |
| 1375426 | linkages 3-4, | 4.1 | 41 | 55 | 4325 |
| | 4-5 | | | | |
| 1375427 | linkages 2-3, | 4.5 | 52 | 29 | 1519 |
| | 3-4 | | | | |
| 1375428 | linkages 1-2, | 4.7 | 38 | 473 | 3945 |
| | 2-3 | | | | |

TABLE 9
In Vivo Dose-response of liver P21 mRNA upon treatment with
modified oligonucleotides complementary CXCL12

| Compound | 1.8 mg/kg | 5.5 mg/kg | 16.7 mg/kg | 50 mg/kg | 150 mg/kg |
|---|---|---|---|---|---|
| ID | Expression level of P21 mRNA (% Control) | | | | |
| 558807 | 48 | 87 | 1148 | 11488 | n.d. |
| 936053 | 98 | 142 | 137 | 132 | 169 |
| 1375426 | 113 | 114 | 106 | 250 | 6388 |
| 1375427 | 79 | 230 | 179 | 180 | 2171 |
| 1375428 | 142 | 111 | 45 | 788 | 12412 |

TABLE 10
In Vivo Dose-response of kidney P21 mRNA upon treatment
with modified oligonucleotides complementary to CXCL12

| Compound | 1.8 mg/kg | 5.5 mg/kg | 16.7 mg/kg | 50 mg/kg | 150 mg/kg |
|---|---|---|---|---|---|
| ID | Expression level of P21 mRNA (% Control) | | | | |
| 558807 | 108 | 140 | 149 | 649 | n.d. |
| 936053 | 100 | 162 | 151 | 105 | 122 |
| 1375426 | 125 | 151 | 132 | 133 | 156 |
| 1375427 | 156 | 170 | 167 | 122 | 208 |
| 1375428 | 143 | 116 | 104 | 109 | 237 |

TABLE 11
In Vivo Dose-response of liver Tnfrsf10b mRNA upon treatment
with modified oligonucleotides complementary to CXCL12

| Compound | 1.8 mg/kg | 5.5 mg/kg | 16.7 mg/kg | 50 mg/kg | 150 mg/kg |
|---|---|---|---|---|---|
| ID | Expression level of P21 mRNA (% Control) | | | | |
| 558807 | 96 | 122 | 870 | 11757 | n.d. |
| 936053 | 151 | 121 | 132 | 148 | 191 |
| 1375426 | 124 | 150 | 115 | 213 | 4869 |
| 1375427 | 116 | 178 | 127 | 280 | 971 |
| 1375428 | 149 | 104 | 118 | 586 | 12528 |

### Example 7: Design, Activity and Tolerability of Modified Oligonucleotides Complementary to SOD1 with Mesyl Phosphoramidate Internucleoside Linkages In Vitro

### Modified Oligonucleotides

Modified oligonucleotides comprising two consecutive mesyl phosphoramidate internucleoside linkages (Formula IX) were synthesized and tested. The modified oligonucleotides are each 3-10-3 cEt gapmers with a sugar motif of: kkkddddddddddkkk (a 3-10-3 cEt motif) where “k” represents a cEt modified sugar moiety, and “d” represents a β-D-2′-deoxyribosyl sugar moiety. Each internucleoside linkage is either a phosphorothioate internucleoside linkage (“s”) or a mesyl phosphoramidate internucleoside linkage (“z”). Each of the compounds in the tables below has two consecutive internucleoside linkages of formula IX.

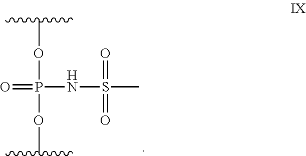 [SMILES: *OP(=O)(NS(C)(=O)=O)O*]

The compounds in the table below have the sequence TGAGGTCCTGCACTGG (SEQ ID NO: 11) and are 100% complementary to mouse SOD1, GENBANK NT_039625.7 truncated from 24924000to 24933000 (SEQ ID NO: 7), at position 5685 to 5880.

### In Vitro Activity Assay

The modified oligonucleotides were tested for their ability to reduce target RNA in a series of experiments. Cultured mouse 3T3-L1 cells at a density of 20,000 cells per well were transfected using electroporation with modified oligonucleotides diluted to 20 μM, 7 μM, 2 μM, 0.7 μM, 0.3 μM, 0.1 μM, and 0.03 μM. After a treatment period of approximately 16 hours, RNA levels were measured using mouse primer-probe set RTS3025 (SOD1; forward sequence: TTTTTTGCGCGGTCCTTTC (SEQ ID NO: 8); reverse sequence: GAGGGACCAGAGAGAGCAAGAC (SEQ ID NO: 9), probe sequence: CGCCTTCCGTCCGTCGGCT (SEQ ID NO: 10)). RNA levels for each target were normalized to total RNA content, as measured by RIBOGREEN®. Activity expressed as half maximal inhibitory concentration (IC50) was calculated using the log (inhibitor) vs response (three parameter) function in GraphPad Prism 7.

In vitro Toxicity Assay

In vitro toxicity of modified oligonucleotides described above was determined as described in Example 3.

TABLE 12
Design, Activity, and Toxicity of modified oligonucleotides having
two consecutive mesyl phosphoramidates linkages complementary to SOD1

| | | SOD1 | Caspase | |
|---|---|---|---|---|
| Compound | | IC₅₀ | Activation | SEQ ID |
| Number | Chemistry Notation (5′-3′) | (μM) | (% Mock) | NO. |
| 508031 | TₖₛGₖₛAₖₛG_{ds}G_{ds}T_{ds}^{m}C_{ds}^{m}C_{ds}T_{ds}G_{ds}^{m}C_{ds}A_{ds}^{m}C_{ds}TₖₛGₖₛGₖ | 0.69 | 902 | 11 |
| 1405473 | TₖₛGₖₛAₖₛG_{ds}G_{dz}T_{dz}^{m}C_{ds}^{m}C_{ds}T_{ds}G_{ds}^{m}C_{ds}A_{ds}^{m}C_{ds}TₖₛGₖₛGₖ | 0.38 | 273 | 11 |
| 1405474 | TₖₛGₖₛAₖₛG_{ds}G_{ds}T_{dz}^{m}C_{dz}^{m}C_{ds}T_{ds}G_{ds}^{m}C_{ds}A_{ds}^{m}C_{ds}TₖₛGₖₛGₖ | 0.33 | 345 | 11 |
| 1405475 | TₖₛGₖₛAₖₛG_{ds}G_{ds}T_{ds}^{m}C_{ds}^{m}C_{ds}T_{ds}G_{dz}^{m}C_{dz}A_{ds}^{m}C_{ds}TₖₛGₖₛGₖ | 0.47 | 844 | 11 |

A subscript “k” represents a cEt nucleoside, a subscript “d” represents a stereostandard DNA nucleoside, a subscript “s” indicates a phosphorothioate internucleoside linkage, a subscript “z” represents an internucleoside linkage of formula IX, which is a mesyl phosphoramidate linkage. Subscripts of nucleotides having an internucleoside linkage offormula IX are bold and underlined. A superscript “m” before a C represents a 5-methyl Cytosine.

### Example 8: Design, Activity and Tolerability of Modified Oligonucleotides Complementary to HDAC2 with Mesyl Phosphoramidate Internucleoside Linkages In Vitro

### Modified Oligonucleotides

Modified oligonucleotides comprising two consecutive mesyl phosphoramidate internucleoside linkages (Formula IX) were synthesized and tested. The modified oligonucleotides are each 3-10-3 cEt gapmers with a sugar motif of: kkkddddddddddkkk (a 3-10-3 cEt motif) where “k” represents a cEt modified sugar moiety, and “d” represents a β-D-2′-deoxyribosyl sugar moiety. Each internucleoside linkage is either a phosphorothioate internucleoside linkage (“s”) or a mesyl phosphoramidate internucleoside linkage (“z”). Each of the compounds in the tables below has two consecutive internucleoside linkages of formula IX.

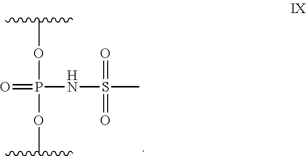 [SMILES: *OP(=O)(NS(C)(=O)=O)O*]

The compounds in the table below are 100% complementary to mouse HDAC2, GENBANK NC_000076.6 truncated from 36972001 to 37005000 (SEQ ID NO: 12), at several positions, as indicated in the table below.

TABLE 13
Positions of modified oligonucleotides
complementary to HDAC2

| Compound | Sequence | Start | Stop | SEQ |
|---|---|---|---|---|
| Number | (5′ to 3′) | Site | Site | ID NO: |
| 546108 | TAGTCTCTGTCAGTTA | 8162 | 8177 | 13 |
| | | 8204 | 8219 | |
| | | 8246 | 8261 | |
| | | 8330 | 8345 | |
| 546110 | TCATGTACCTATAGTC | 8173 | 8188 | 14 |
| | | 8215 | 8230 | |
| | | 8257 | 8272 | |
| | | 8299 | 8314 | |
| | | 8341 | 8356 | |

### In Vitro Activity Assay

The modified oligonucleotides were tested for their ability to reduce target RNA in a series of experiments. Cultured mouse 3T3-L1 cells at a density of 20,000 cells per well were transfected using electroporation with modified oligonucleotides diluted to 20 μM, 7 μM, 2 μM, 0.7 μM, 0.3 μM, 0.1 μM, and 0.03 μM. After a treatment period of approximately 16 hours, RNA levels were measured using mouse HDAC2 primer-probe set RTS3500 (forward sequence TGATGGTGTTGAGGAAGCTTTTT (SEQ ID NO: 15, reverse sequence: TCCCTCAAGTCTCCTGTTCCA (SEQ TD NO: 16), probe sequence: ACAACAGATCGCGTGATGACCGTCTC, (SEQ ID NO: 17)). RNA levels for each5target were normalized to total RNA content, as measured by RIBOGREEN Activity expressed as half maximal inhibitory concentration (IC₅₀) was calculated using the log (inhibitor) vs response (three parameter) function in GraphPad Prism 7.

In vitro Toxicity Assay

Invitro toxicity of modified oligonucleotides described above was determined as described in Example 3.

TABLE 14
Modified oligonucleotides having two consecutive mesyl
phosphoramidate linkages complementary to HDAC2

| | | HDAC2 | Caspase | |
|---|---|---|---|---|
| Compound | | IC₅₀ | Activation | SEQ |
| Number | Chemistry Notation (5′-3′) | (μM) | (% Mock) | ID NO. |
| 546108 | TₖₛAₖₛGₖₛT_{ds}^{m}C_{ds}T_{ds}^{m}C_{ds}T_{ds}G_{ds}T_{ds}^{m}C_{ds}A_{ds}G_{ds}TₖₛTₖₛAₖ | 0.57 | 602 | 13 |
| 1405476 | TₖₛAₖₛGₖₛT_{ds}^{m}C_{dz}T_{dz}^{m}C_{ds}T_{ds}G_{ds}T_{ds}^{m}C_{ds}A_{ds}G_{ds}TₖₛTₖₛAₖ | 0.24 | 178 | 13 |
| 1405477 | TₖₛAₖₛGₖₛT_{ds}^{m}C_{ds}T_{dz}^{m}C_{dz}T_{ds}G_{ds}T_{ds}^{m}C_{ds}A_{ds}G_{ds}TₖₛTₖₛAₖ | 0.31 | 225 | 13 |
| 1405478 | TₖₛAₖₛGₖₛT_{ds}^{m}C_{ds}T_{ds}^{m}C_{ds}T_{ds}G_{ds}T_{dz}^{m}C_{dz}A_{ds}G_{ds}TₖₛTₖₛAₖ | 0.28 | 207 | 13 |
| 546110 | Tₖₛ^{m}CₖₛAₖₛT_{ds}G_{ds}T_{ds}A_{ds}^{m}C_{ds}^{m}C_{ds}T_{ds}A_{ds}T_{ds}A_{ds}GₖₛTₖₛ^{m}Cₖ | 0.20 | 142 | 14 |
| 1405479 | Tₖₛ^{m}CₖₛAₖₛT_{ds}G_{dz}T_{dz}A_{ds}^{m}C_{ds}^{m}C_{ds}T_{ds}A_{ds}T_{ds}A_{ds}GₖₛTₖₛ^{m}Cₖ | 0.61 | 74 | 14 |
| 1405480 | Tₖₛ^{m}CₖₛAₖₛT_{ds}G_{ds}T_{dz}A_{dz}^{m}C_{ds}^{m}C_{ds}T_{ds}A_{ds}T_{ds}A_{ds}GₖₛTₖₛ^{m}Cₖ | 0.43 | 79 | 14 |
| 1405481 | Tₖₛ^{m}CₖₛAₖₛT_{ds}G_{ds}T_{ds}A_{ds}^{m}C_{ds}^{m}C_{ds}T_{dz}A_{dz}T_{ds}A_{ds}GₖₛTₖₛ^{m}Cₖ | 0.63 | 113 | 14 |

A subscript “k” represents a cEt nucleoside, a subscript “d” represents a stereostandard DNA nucleoside, a subscript “s” indicates a phosphorothioate internucleoside linkage, a subscript “z” represents an internucleoside linkage of formula IX, which is a mesyl phosphoramidate linkage. Subscripts of nucleotides having an internucleoside linkage of formula IX are bold and underlined. A superscript “m” before a C represents a 5-methyl Cytosine.

A subscript “k” represents a cEt nucleoside, a subscript “d” represents a stereostandard DNA nucleoside, a subscript “s” indicates a phosphorothioate internucleoside linkage, a subscript “z” represents an internucleoside linkage of formula IX, which is a mesyl phosphoramidate linkage. Subscripts of nucleotides having an internucleoside linkage of formula IX are bold and underlined. A superscript “m” before a C represents a 5-methyl Cytosine.

### Example 9: Design, Activity and Tolerability of Modified Oligonucleotides Having Multiple Mesyl Phosphoramidate Internucleoside Linkages In Vitro

### Modified Oligonucleotides

Modified oligonucleotides comprising multiple mesyl phosphoramidate internucleoside linkages (Formula IX) were synthesized and tested. The modified oligonucleotides are each 3-10-3 cEt gapmers with a sugar motif of: kkkddddddddddkkk (a 3-10-3 cEt motif) where “k” represents a cEt modified sugar moiety, and “d” represents a β-D-2′-deoxyribosyl sugar moiety. Each of the modified oligonucleotides has the same nucleobase sequence, GCATGTTCTCACATTA (SEQ ID NO: 5), which is 100% complementary to mouse CXCL12, GENBANK NT_039353.7 truncated from 69/430,515 to 69/445,350 (SEQ ID NO: 1), at position 6877 to 6892. Each internucleoside linkage is either a phosphorothioate internucleoside linkage (“s”) or a mesyl phosphoramidate internucleoside linkage (“z”).

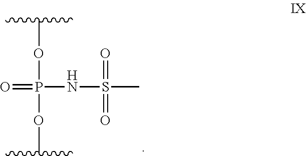 [SMILES: *OP(=O)(NS(C)(=O)=O)O*]

### In Vitro Assays

In vitro activity of modified oligonucleotides described above was determined as described in Example 1. In vitro toxicity of modified oligonucleotides described above was determined as described in Example 3.

Each of the compounds in the table below has three or four consecutive internucleoside linkages of formula IX.

TABLE 15
Design, Activity, and Tolerability of modified oligonucleotides
having three or four consecutive mesyl phosphoramidates linkages
complementary to CXCL12

| | | CXCL12 | Caspase | |
|---|---|---|---|---|
| Compound | | IC₅₀ | Activation | SEQ |
| Number | Chemistry Notation (5′-3′) | (μM) | (% Mock) | ID NO. |
| 558807 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 0.17 | 1329 | 5 |
| 1405434 | Gₖₛ^{m}CₖₛAₖₛT_{dz}G_{dz}T_{dz}T_{ds}^{m}C_{ds}T_{ds}^{m}C_{ds}A_{ds}^{m}C_{ds}A_{ds}TₖₛTₖₛAₖ | 0.16 | 211 | 5 |
| 1405435 | Gₖₛ^{m}CₖₛAₖₛT_{ds}G_{dz}T_{dz}T_{dz}^{m}C_{ds}T_{ds}^{m}C_{ds}A_{ds}^{m}C_{ds}A_{ds}TₖₛTₖₛAₖ | 0.12 | 240 | 5 |
| 1405436 | Gₖₛ^{m}CₖₛAₖₛT_{dz}G_{dz}T_{dz}T_{dz}^{m}C_{ds}T_{ds}^{m}C_{ds}A_{ds}^{m}C_{ds}A_{ds}TₖₛTₖₛAₖ | 0.12 | 187 | 5 |

A subscript “k” represents a cEt nucleoside, a subscript “d” represents a stereostandard DNA nucleoside, a subscript “s” indicates a phosphorothioate internucleoside linkage, a subscript “z” represents an internucleoside linkage of formula IX, which is a mesyl phosphoramidate linkage. Subscripts of nucleotides having an internucleoside linkage of formula IX are bold and underlined. A superscript “m” before a C represents a 5-methyl Cytosine.

Each of the compounds in the table below has multiple internucleoside linkages of formula IX.

TABLE 16
Design, Activity, and Tolerability of modified oligonucleotides having
multiple mesyl phosphoramidate linkages complementary to CXCL12

| | | CXCL1 | Caspase | |
|---|---|---|---|---|
| Compound | | IC₅₀ | Activation | SEQ ID |
| Number | Chemistry Notation (5′-3′) | (μM) | (% Mock) | NO. |
| 558807 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 0.20 | 2727 | 5 |
| 1437592 | Gₖₛ^{m}CₖₛA_{kz}T_{dz}G_{dz}T_{ds}T_{ds}^{m}C_{ds}T_{ds}^{m}C_{ds}A_{ds}^{m}C_{ds}A_{ds}TₖₛTₖₛAₖ | 0.11 | 1708 | 5 |
| 1437593 | Gₖₛ^{m}CₖₛA_{kz}T_{dz}G_{dz}T_{dz}T_{ds}^{m}C_{ds}T_{ds}^{m}C_{ds}A_{ds}^{m}C_{ds}A_{ds}TₖₛTₖₛAₖ | 0.15 | 500 | 5 |
| 1437594 | Gₖₛ^{m}CₖₛA_{kz}T_{dz}G_{dz}T_{dz}T_{dz}^{m}C_{ds}T_{ds}^{m}C_{ds}A_{ds}^{m}C_{ds}A_{ds}TₖₛTₖₛAₖ | 0.24 | 308 | 5 |
| 1437595 | Gₖₛ^{m}CₖₛA_{kz}T_{dz}G_{dz}T_{dz}T_{dz}^{m}C_{dz}T_{ds}^{m}C_{ds}A_{ds}^{m}C_{ds}A_{ds}TₖₛTₖₛAₖ | 0.26 | 309 | 5 |
| 1437596 | Gₖₛ^{m}CₖₛA_{kz}T_{dz}G_{dz}T_{dz}T_{dz}^{m}C_{dz}T_{dz}^{m}C_{ds}A_{ds}^{m}C_{ds}A_{ds}TₖₛTₖₛAₖ | 0.50 | 324 | 5 |
| 1437597 | Gₖₛ^{m}CₖₛA_{kz}T_{dz}G_{dz}T_{dz}T_{dz}^{m}C_{dz}T_{dz}^{m}C_{dz}A_{ds}^{m}C_{ds}A_{ds}TₖₛTₖₛAₖ | 0.28 | 193 | 5 |
| 1437598 | Gₖₛ^{m}CₖₛA_{kz}T_{dz}G_{dz}T_{dz}T_{dz}^{m}C_{dz}T_{dz}^{m}C_{dz}A_{dz}^{m}C_{ds}A_{ds}TₖₛTₖₛAₖ | 0.44 | 181 | 5 |
| 1437599 | Gₖₛ^{m}CₖₛAₖₛT_{ds}G_{ds}T_{ds}T_{ds}^{m}C_{ds}T_{ds}^{m}C_{ds}A_{dz}^{m}C_{dz}A_{dz}TₖₛTₖₛAₖ | 0.16 | 1290 | 5 |
| 1437600 | Gₖₛ^{m}CₖₛAₖₛT_{ds}G_{ds}T_{ds}T_{ds}^{m}C_{ds}T_{ds}^{m}C_{dz}A_{dz}^{m}C_{dz}A_{dz}TₖₛTₖₛAₖ | 0.14 | 1807 | 5 |
| 1437601 | Gₖₛ^{m}CₖₛAₖₛT_{ds}G_{ds}T_{ds}T_{ds}^{m}C_{ds}T_{dz}^{m}C_{dz}A_{dz}^{m}C_{dz}A_{dz}TₖₛTₖₛAₖ | 0.18 | 1941 | 5 |
| 1437602 | Gₖₛ^{m}CₖₛAₖₛT_{ds}G_{ds}T_{ds}T_{ds}^{m}C_{dz}T_{dz}^{m}C_{dz}A_{dz}^{m}C_{dz}A_{dz}TₖₛTₖₛAₖ | 0.13 | 1927 | 5 |
| 1437603 | Gₖₛ^{m}CₖₛAₖₛT_{ds}G_{ds}T_{ds}T_{dz}^{m}C_{dz}T_{dz}^{m}C_{dz}A_{dz}^{m}C_{dz}A_{dz}TₖₛTₖₛAₖ | 0.32 | 866 | 5 |
| 1437604 | Gₖₛ^{m}CₖₛAₖₛT_{ds}G_{ds}T_{dz}T_{dz}^{m}C_{dz}T_{dz}^{m}C_{dz}A_{dz}^{m}C_{dz}A_{dz}TₖₛTₖₛAₖ | 0.97 | 250 | 5 |
| 1437605 | Gₖₛ^{m}CₖₛAₖₛT_{ds}G_{dz}T_{dz}T_{dz}^{m}C_{dz}T_{dz}^{m}C_{dz}A_{dz}^{m}C_{dz}A_{dz}TₖₛTₖₛAₖ | 1.84 | 152 | 5 |
| 1437606 | Gₖₛ^{m}CₖₛAₖₛT_{dz}G_{dz}T_{dz}T_{dz}^{m}C_{dz}T_{dz}^{m}C_{dz}A_{dz}^{m}C_{dz}A_{dz}TₖₛTₖₛAₖ | 1.69 | 155 | 5 |
| 1441068 | Gₖₛ^{m}CₖₛA_{kz}T_{ds}G_{dz}T_{ds}T_{dz}^{m}C_{ds}T_{dz}^{m}C_{ds}A_{dz}^{m}C_{ds}A_{dz}TₖₛTₖₛAₖ | 0.29 | 759 | 5 |
| 1441069 | Gₖₛ^{m}CₖₛAₖₛT_{dz}G_{ds}T_{dz}T_{ds}^{m}C_{dz}T_{ds}^{m}C_{dz}A_{ds}^{m}C_{dz}A_{ds}TₖₛTₖₛAₖ | 0.39 | 1122 | 5 |

A subscript “k” represents a cEt nucleoside, a subscript “d” represents a stereostandard DNA nucleoside, a subscript “s” indicates a phosphorothioate internucleoside linkage, a subscript “z” represents an internucleoside linkage of formula IX, which is a mesyl phosphoramidate linkage. Subscripts of nucleotides having an internucleoside linkage of formula IX are bold and underlined. A superscript “m” before a C represents a 5-methyl Cytosine.

### Example 10: Design, Synthesis, Activity, and Tolerability of Modified Oligonucleotides Having Various Modified Phosphoramidate Internucleoside Linkages In Vitro

Modified oligonucleotides comprising mesyl phosphoramidate internucleoside linkages were synthesized and tested. The modified oligonucleotides are each 3-10-3 cEt gapmers with a sugar motif of (from 5′ to 3′): kkkddddddddddkkk (a 3-10-3 cEt motif) wherein “k” represents a cEt modified sugar moiety, and “d” represents a β-D-2′-deoxyribosyl sugar moiety. Each of the modified oligonucleotides has the same nucleobase sequence, GCATGTTCTCACATTA (SEQ ID NO: 5), which is 100% complementary to mouse CXCL12, GENBANK NT_039353.7 truncated from 69/430,515 to 69/445,350 (SEQ ID NO: 1), at position 6877 to 6892. Each internucleoside linkage is either a phosphorothioate internucleoside linkage (“s”) or a modified phosphoramidate internucleoside linkage represented by formulas X-XVI, as indicated in the table below.

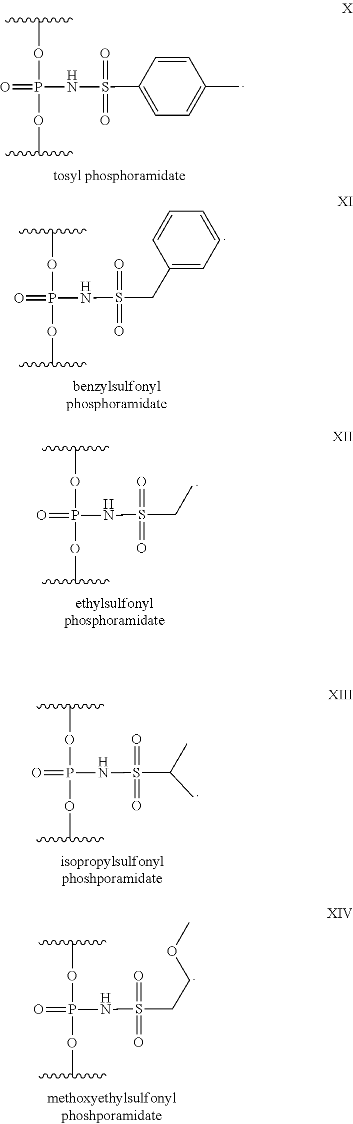 [SMILES: *OP(=O)(NS(=O)(=O)c1ccc(C)cc1)O* ; *OP(=O)(NS(=O)(=O)Cc1ccccc1)O* ; *OP(=O)(NS(=O)(=O)CC)O* ; *OP(=O)(NS(=O)(=O)C(C)C)O* ; *OP(=O)(NS(=O)(=O)CCOC)O*]

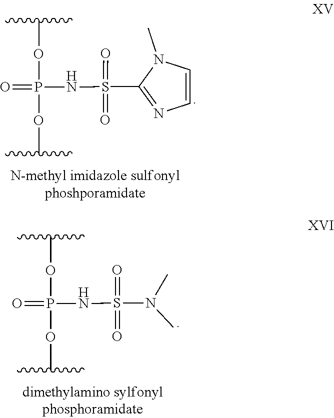 [SMILES: *OP(=O)(NS(=O)(=O)c1nccn1C)O* ; *OP(=O)(NS(=O)(=O)N(C)C)O*]

### Synthesis

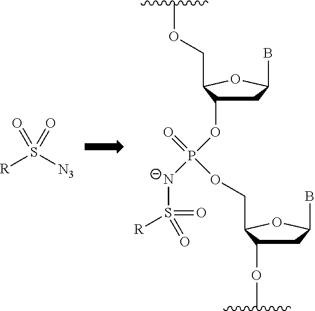

Oligonucleotides were synthesized on a 2 μmol scale using VIMAD UnyLinker support (200 μmol/g) on an ABI 394 DNA/RNA synthesizer. Fully protected nucleoside phosphoramidites were incorporated using standard solid-phase oligonucleotide synthesis, i.e. 3% dichloroacetic acid in dichloromethane for deblocking, 1 M 4,5-dicyanoimidazole 0.1 M N-methylimidazole in acetonitrile as activator for amidite couplings, 20% acetic anhydride in THF and 10% 1-methylimidazole in THF/pyridine for capping and 0.1 M xanthane hydride in pyridine:acetonitrile 3:2 (v:v) for thiolation. Mesyl phosphoramidate couplings were oxidized instead of thiolated using 0.5 M mesyl azide in acetonitrile:toluene 1:1 (v:v) with oxidation times varying (3×500s to 6×900s) depending on the steric hindrance of the substituted azide or the steric hindrance of the phosphoramidite being oxidized (Table 17 and Table 18). Amidites were dissolved to 0.1 M in acetonitrile:toluene 1:1 (v:v) and incorporated using 6 min coupling recycling time for DNA amidites and 10 min for all other amidites. At the end of the solid phase synthesis cyanoethyl protecting groups were removed by a 30 min treatment with 20% diethylamine in toluene. Modified oligonucleotides were deprotected and cleaved using conc. aq. ammonia at room temperature for 48 h or at 55° C. overnight.

TABLE 17
Oxidation times for the various substituted azide analogs
using an ABI oligonucleotides synthesizer on 2 μmol scale

| Substituted Azide | R = | Oxidation time |
|---|---|---|
| | methyl | 3 × 500 s |
| | ethyl | 3 × 500 s |
| | methoxy ethyl | 3 × 650 s |
| | isopropyl | 3 × 999 s |
| | tosyl | 3 × 500 s |
| 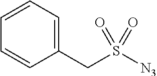 [SMILES: [N-]=[N+]=NS(=O)(=O)Cc1ccccc1] | benzyl | 3 × 500 s |
| 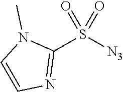 | N-methyl imidazole | 3 × 500 s |
| 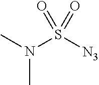 | dimethylamine | 4 × 900 s |

TABLE 18
Oxidation times for various sugar phosphoramidites
to form mesyl phosphoramidate linkages (R = methyl)

| Sugar modification | Oxidation time |
|---|---|
| DNA | 3 × 500 s |
| cEt | 6 × 900 s |
| OMe | 3 × 650 s |
| MOE | 3 × 650 s |
| 2′-F | 4 × 750 s |
| LNA | 6 × 900 s |

### In Vitro Assays

In vitro activity of modified oligonucleotides described above was determined as described in Example 1. In vitro toxicity of modified oligonucleotides described above was determined as described in Example 3. Each experiment is presented in a separate table.

TABLE 19
Design, activity, and tolerability of modified oligonucleotides having modified
phosphoramidate linkages of formulas X, XI, or XII complementary to CXCL12

| | | CXCL12 | Caspase | |
|---|---|---|---|---|
| Compound | | IC₅₀ | Activation | SEQ |
| Number | Chemistry Notation (5′-3′) | (nM) | (% Mock) | ID NO. |
| 558807 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 150 | 1336 | 5 |
| 1419483 | Gₖₛ^{m}CₖₛAₖₛT_{dX}G_{dX}T_{ds}T_{ds}^{m}C_{ds}T_{ds}^{m}C_{ds}A_{ds}^{m}C_{ds}A_{ds}TₖₛTₖₛAₖ | 263 | 141 | 5 |
| 1419482 | Gₖₛ^{m}CₖₛAₖₛT_{ds}G_{dX}T_{dX}T_{ds}^{m}C_{ds}T_{ds}^{m}C_{ds}A_{ds}^{m}C_{ds}A_{ds}TₖₛTₖₛAₖ | 299 | 88 | 5 |
| 1419481 | Gₖₛ^{m}CₖₛAₖₛT_{ds}G_{ds}T_{dX}T_{dX}^{m}C_{ds}T_{ds}^{m}C_{ds}A_{ds}^{m}C_{ds}A_{ds}TₖₛTₖₛAₖ | 404 | 79 | 5 |
| 1419480 | Gₖₛ^{m}CₖₛAₖₛT_{ds}G_{ds}T_{ds}T_{ds}^{m}C_{ds}T_{ds}^{m}C_{dX}A_{dX}^{m}C_{ds}A_{ds}TₖₛTₖₛAₖ | 171 | 433 | 5 |
| 1427921 | Gₖₛ^{m}CₖₛAₖₛT_{dXI}G_{dXI}T_{ds}T_{ds}^{m}C_{ds}T_{ds}^{m}C_{ds}A_{ds}^{m}C_{ds}A_{ds}TₖₛTₖₛAₖ | 439 | 134 | 5 |
| 1427922 | Gₖₛ^{m}CₖₛAₖₛT_{ds}T_{dXI}G_{dXI}T_{ds}^{m}C_{ds}T_{ds}^{m}C_{ds}A_{ds}^{m}C_{ds}A_{ds}TₖₛTₖₛAₖ | 431 | 109 | 5 |
| 1427923 | Gₖₛ^{m}CₖₛAₖₛT_{ds}G_{ds}T_{dXI}T_{dXI}^{m}C_{ds}T_{ds}^{m}C_{ds}A_{ds}^{m}C_{ds}A_{ds}TₖₛTₖₛAₖ | 465 | 90 | 5 |
| 1427924 | Gₖₛ^{m}CₖₛAₖₛT_{ds}G_{ds}T_{ds}T_{ds}^{m}C_{ds}T_{ds}^{m}C_{dXI}A_{dXI}^{m}C_{ds}A_{ds}TₖₛTₖₛAₖ | 181 | 517 | 5 |
| 1417944 | Gₖₛ^{m}CₖₛAₖₛT_{dXII}G_{dXII}T_{ds}T_{ds}^{m}C_{ds}T_{ds}^{m}C_{ds}A_{ds}^{m}C_{ds}A_{ds}TₖₛTₖₛAₖ | 118 | 982 | 5 |
| 1417943 | Gₖₛ^{m}CₖₛAₖₛT_{ds}G_{dXII}T_{dXII}T_{ds}^{m}C_{ds}T_{ds}^{m}C_{ds}A_{ds}^{m}C_{ds}A_{ds}TₖₛTₖₛAₖ | 136 | 274 | 5 |
| 1417942 | Gₖₛ^{m}CₖₛAₖₛT_{ds}G_{ds}T_{dXII}T_{dXII}^{m}C_{ds}T_{ds}^{m}C_{ds}A_{ds}^{m}C_{ds}A_{ds}TₖₛTₖₛAₖ | 167 | 452 | 5 |
| 1417941 | Gₖₛ^{m}CₖₛAₖₛT_{ds}G_{ds}T_{ds}T_{ds}^{m}C_{ds}T_{ds}^{m}C_{dXII}A_{dXII}^{m}C_{ds}A_{ds}TₖₛTₖₛAₖ | 63 | 2029 | 5 |

A subscript “k” represents a cEt leoside, a subscript “d” represents a stereo-standard DNA nucleoside, a subscript “s” indicates a phosphorothioate internucleoside linkage. A subscript “X” represents an internucleoside linkage of formula X; a subscript “XI” represents an internucleoside linkage of formula XI; a subscript “XII” represents an internucleoside linkage of formula XII. Subscripts of nucleotides having a substituted phosphoramidate internucleoside linkage of generic Formula XVII are bold and underlined. A superscript “m” before a C represents a 5-methyl Cytosine.

TABLE 20
Design, activity, and tolerability of modified oligonucleotides having modified
phosphoramidate linkages of formulas XIII, XIV, or XV complementary to CXCL12

| | | CXCL12 | Caspase | SEQ |
|---|---|---|---|---|
| Compound | | IC₅₀ | Activation | ID |
| Number | Chemistry Notation (5′-3′) | (nM) | (% Mock) | NO. |
| 558807 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 98 | 986 | 5 |
| 1429189 | Gₖₛ^{m}CₖₛAₖₛT_{dXIII}G_{dXIII}T_{ds}T_{ds}^{m}C_{ds}T_{ds}^{m}C_{ds}A_{ds}^{m}C_{ds}A_{ds}TₖₛTₖₛAₖ | 80 | 638 | 5 |
| 1429190 | Gₖₛ^{m}CₖₛAₖₛT_{ds}G_{dXIII}T_{dXIII}T_{ds}^{m}C_{ds}T_{ds}^{m}C_{ds}A_{ds}^{m}C_{ds}A_{ds}TₖₛTₖₛAₖ | 113 | 168 | 5 |
| 1429191 | Gₖₛ^{m}CₖₛAₖₛT_{ds}G_{ds}T_{dXIII}T_{dXIII}^{m}C_{ds}T_{ds}^{m}C_{ds}A_{ds}^{m}C_{ds}A_{ds}TₖₛTₖₛAₖ | 111 | 184 | 5 |
| 1429192 | Gₖₛ^{m}CₖₛAₖₛT_{ds}G_{ds}T_{ds}T_{ds}^{m}C_{ds}T_{ds}^{m}C_{dXIII}A_{dXIII}^{m}C_{ds}A_{ds}TₖₛTₖₛAₖ | 46 | 1004 | 5 |
| 1417948 | GₖₛCₖₛAₖₛT_{dXIV}G_{dXIV}T_{ds}T_{ds}^{m}C_{ds}T_{ds}^{m}C_{ds}A_{ds}^{m}C_{ds}A_{ds}TₖₛTₖₛAₖ | 98 | 1270 | 5 |
| 1417947 | Gₖₛ^{m}CₖₛAₖₛT_{ds}G_{dXIV}T_{dXIV}T_{ds}^{m}C_{ds}T_{ds}^{m}C_{ds}A_{ds}^{m}C_{ds}A_{ds}TₖₛTₖₛAₖ | 141 | 271 | 5 |
| 1417946 | Gₖₛ^{m}CₖₛAₖₛT_{ds}G_{ds}T_{dXIV}T_{dXIV}^{m}C_{ds}T_{ds}^{m}C_{ds}A_{ds}^{m}C_{ds}A_{ds}TₖₛTₖₛAₖ | 128 | 232 | 5 |
| 1417945 | Gₖₛ^{m}CₖₛAₖₛT_{ds}G_{ds}T_{ds}T_{ds}^{m}C_{ds}T_{ds}^{m}C_{dXIV}A_{dXIV}^{m}C_{ds}A_{ds}TₖₛTₖₛAₖ | 276 | 951 | 5 |
| 1431805 | Gₖₛ^{m}CₖₛAₖₛT_{dXV}G_{dXV}T_{ds}T_{ds}^{m}C_{ds}T_{ds}^{m}C_{ds}A_{ds}^{m}C_{ds}A_{ds}TₖₛTₖₛAₖ | 299 | 235 | 5 |
| 1431806 | Gₖₛ^{m}CₖₛAₖₛT_{ds}G_{dXV}T_{dXV}T_{ds}^{m}C_{ds}T_{ds}^{m}C_{ds}A_{ds}^{m}C_{ds}A_{ds}TₖₛTₖₛAₖ | 152 | 137 | 5 |
| 1431807 | Gₖₛ^{m}CₖₛAₖₛT_{ds}G_{ds}T_{dXV}T_{dXV}^{m}C_{ds}T_{ds}^{m}C_{ds}A_{ds}^{m}C_{ds}A_{ds}TₖₛTₖₛAₖ | 243 | 119 | 5 |
| 1431808 | Gₖₛ^{m}CₖₛAₖₛT_{ds}G_{ds}T_{ds}T_{ds}^{m}C_{ds}T_{ds}^{m}C_{dXV}A_{dXV}^{m}C_{ds}A_{ds}TₖₛTₖₛAₖ | 114 | 556 | 5 |

A subscript “k” represents a cEt nucleoside, a subscript “d” represents a stereo-standard DNA nucleoside, a subscript “s” indicates a phosphorothioate internucleoside linkage. A subscript “XIII” represents an internucleoside linkage of formula XIII; a subscript “XIV” represents an internucleoside linkage of formula XIV; a subscript “XV” represents aninternucleoside linkage of formula XV. Subscripts of nucleotides having a modified mesyl phosphoramidate internucleoside linkage of generic Formula XVII are bold and underlined. A superscript “m” before a C represents a 5-methyl Cytosine.

TABLE 21
Design, Activity, and Tolerability of modified oligonucleotides having
modified phosphoramidate linkages of formula XVI complementary to CXCL12

| | | CXCL12 | Caspase | |
|---|---|---|---|---|
| Compound | | IC₅₀ | Activation | SEQ |
| Number | Chemistry Notation (5′-3′) | (nM) | (% Mock) | ID NO. |
| 558807 | Gₕₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 98* | 1026 | 5 |
| 1431745 | Gₕₛ^{m}CₖₛAₖₛT_{dXVI}G_{dXVI}T_{ds}T_{ds}^{m}C_{ds}T_{ds}^{m}C_{ds}A_{ds}^{m}C_{ds}A_{ds}TₖₛTₖₛAₖ | 82 | 786 | 5 |
| 1431746 | Gₕₛ^{m}CₖₛAₖₛT_{ds}G_{dXVI}T_{dXVI}T_{ds}^{m}C_{ds}T_{ds}^{m}C_{ds}A_{ds}^{m}C_{ds}A_{ds}TₖₛTₖₛAₖ | 139 | 344 | 5 |
| 1431747 | Gₕₛ^{m}CₖₛAₖₛT_{ds}G_{ds}T_{dXVI}T_{dXVI}^{m}C_{ds}T_{ds}^{m}C_{ds}A_{ds}^{m}C_{ds}A_{ds}TₖₛTₖₛAₖ | 137 | 317 | 5 |
| 1431748 | Gₕₛ^{m}CₖₛAₖₛT_{ds}G_{ds}T_{ds}T_{ds}^{m}C_{ds}T_{ds}^{m}C_{dXVI}A_{dXVI}^{m}C_{ds}A_{ds}TₖₛTₖₛAₖ | 82 | 1157 | 5 |

*historical data; not determined in this experiment.
A subscript “k” represents a cEt nucleoside, a subscript “d” represents a stereo-standard DNA nucleoside, a subscript “s” indicates a phosphorothioate internucleoside linkage. A subscript “XVI” represents an internucleoside linkage of formula XVI. Subscripts of nucleotides having a substituted phosphoramidate internucleoside linkage of generic Formula XVII are bold and underlined. A superscript “m” before a C represents a 5-methyl Cytosine.

### Example 11: Design and Protein Upregulation Activity of Uniformly 2′-Modified Oligonucleotides Having Mesyl Phosphoramidate Internucleoside Linkages In Vitro

### Modified Oligonucleotides

Modified oligonucleotides comprising multiple mesyl phosphoramidate internucleoside linkages (Formula IX) were synthesized and tested. The modified oligonucleotides are uniform 2′-OMe modified oligonucleotides. Each of the modified oligonucleotides has the same nucleobase sequence, TGCAGTGGGGTGATTT (SEQ ID NO: 18), which is 100% complementary to human LDLR mRNA GenBank NM_000527.4. (SEQ ID NO: 19), at position 28 to 43. Each internucleoside linkage is either a phosphorothioate internucleoside linkage (“s”) or a mesyl phosphoramidate internucleoside linkage (“z”).

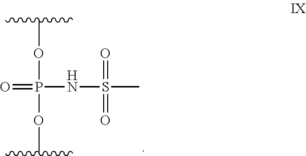 [SMILES: *OP(=O)(NS(C)(=O)=O)O*]

### Protein Upregulation

Modified oligonucleotides were tested for their ability to upregulate LDLR protein after transfection in HeLa cells. Compound No. 842196 is a uniform 2′-OMe/phosphorothioate oligonucleotide that upregulates expression of LDLR (Liang, et. al., Nucleic Acids Research 2017). Cells were transfected using 25 nM of modified oligonucleotide and Lipofectamine® 2000 (Invitrogen) for 16 hours. Cells were harvested and LDLR protein was quantified using the Quantikine ELISA Human LDLR Kit (Biotechne, Catalog Number: DLDLR0), normalized to the expression level of untreated control cells. The results show that modified oligonucleotides comprising mesyl phosphoramidate linkages at the 5′ end are more effective than a full phosphorothioate counterpart for the upregulation of LDLR.

TABLE 22
Design and Activity (protein upregulation) of modified
oligonucleotides having mesyl phosphoramidate
linkages of formula IX complementary to LDLR

| | | Relative | |
|---|---|---|---|
| | | LDLR | SEQ |
| Compound | | Protein | ID |
| Number | Chemistry Notation (5′-3′) | Level | NO. |
| 842196 | U_{ys}G_{ys}C_{ys}A_{ys}G_{ys}U_{ys}G_{ys}G_{ys}G_{ys}G_{ys}U_{ys}G_{ys}A_{ys}U_{ys}U_{ys}U_{y} | 189 | 20 |
| 1405447 | U_{yz}G_{yz}C_{yz}A_{yz}G_{yz}U_{yz}G_{yz}G_{yz}G_{yz}G_{yz}U_{yz}G_{yz}A_{yz}U_{yz}U_{yz}U_{yz} | 146 | 20 |
| 1405546 | U_{yz}G_{yz}C_{yz}A_{yz}G_{yz}U_{yz}G_{ys}G_{ys}G_{ys}G_{ys}U_{ys}G_{ys}A_{ys}U_{ys}U_{ys}U_{y} | 235 | 20 |
| 1405547 | U_{ys}G_{ys}C_{ys}A_{ys}G_{ys}U_{yz}G_{yz}G_{yz}G_{yz}G_{yz}U_{yz}G_{ys}A_{ys}U_{ys}U_{ys}U_{y} | 181 | 20 |
| 1405548 | U_{ys}G_{ys}C_{ys}A_{ys}G_{ys}U_{ys}G_{ys}G_{ys}G_{ys}G_{yz}U_{yz}G_{yz}A_{yz}U_{yz}U_{yz}U_{y} | 133 | 20 |

A subscript “y” represents a 2′-OMe modified nucleoside, a subscript “s” indicates a phosphorothioate internucleoside linkage, a subscript “z” represents an internucleoside linkage of formula IX, which is a mesyl phosphoramidate linkage. Subscripts of nucleotides having a phosphoramidate internucleoside linkage of generic Formula XVII are bold and underlined. A superscript “m” before a C represents a 5-methyl Cytosine.

### Example 12: Design and Activity of siRNA to HRPT1 Having Mesyl Phosphoramidate Internucleoside Linkages In Vitro

siRNA

Double-stranded siRNA comprising modified oligonucleotides having mesyl phosphoramidate internucleoside linkages (Formula IX) in the sense and/or antisense strands were synthesized and tested. Each internucleoside linkage is either a phosphorothioate internucleoside linkage (“s”), a phosphodiester internucleoside linkage (“o”), or a mesyl phosphoramidate internucleoside linkage (“z”), indicated by formula IX below.

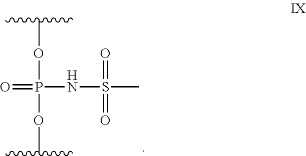 [SMILES: *OP(=O)(NS(C)(=O)=O)O*]

Each antisense strand has the sequence AUAAAAUCUACAGUCAUAGGAAU (SEQ ID NO: 21) and is 100% complementary to GenBank NM_000194.2 (SEQ ID NO: 22) from 444 to 466, and each antisense strand has a 5′-phosphate. Each sense strand has the sequence UCCUAUGACUGUAGAUUUUAU (SEQ ID NO: 23) and is 100% identical to GenBank NM_000194.2 (SEQ ID NO: 22) from 446 to466. Compound No. 1151789 further comprises a 3′-linked C₇ amino modifier (Glen Research), shown below:

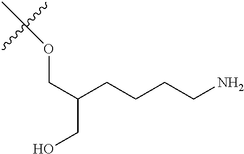 [SMILES: *OCC(CO)CCCCN]

TABLE 23
Design of antisense strand modified oligonucleotides targeted to
human/mouse HRPT1 having mesyl phosphoramidate linkages

| Compound | | SEQ ID |
|---|---|---|
| ID | Chemical Notation (5′ to 3′) | NO: |
| 1073762 | p.A_{yo}U_{fo}A_{yo}A_{fo}A_{yo}A_{fo}U_{yo}C_{fo}U_{yo}A_{fo}C_{yo}A_{fo}G_{yo}U_{fo}C_{yo}A_{fo}U_{yo}A_{fo}G_{yo}G_{fo}A_{ys}AfₛU_{y} | 21 |
| 1405420 | p.A_{yo}U_{fo}A_{yo}A_{fo}A_{yo}A_{fo}U_{yo}C_{fo}U_{yo}A_{fo}C_{yo}A_{fo}G_{yo}U_{fo}C_{yo}A_{fo}U_{yo}A_{fo}G_{yo}G_{fo}A_{yz}A_{fz}U_{y} | 21 |
| 1405427 | p.A_{yz}U_{fz}A_{yo}A_{fo}A_{yo}A_{fo}U_{yo}C_{fo}U_{yo}A_{fo}C_{yo}A_{fo}G_{yo}U_{fo}C_{yo}A_{fo}U_{yo}A_{fo}G_{yo}G_{fo}A_{yo}A_{fo}U_{y} | 21 |
| 1405428 | p.A_{yz}U_{fz}A_{yo}A_{fo}A_{yo}A_{fo}U_{yo}C_{fo}U_{yo}A_{fo}C_{yo}A_{fo}G_{yo}U_{fo}C_{yo}A_{fo}U_{yo}A_{fo}G_{yo}G_{fo}A_{yz}A_{fz}U_{y} | 21 |

A “p.” represents a 5′-phosphate. A subscript “f” represents a 2′-F modified nucleoside, a subscript “y” represents a 2′-OMe modified nucleoside, a subscript “s” indicates a phosphorothioate internucleoside linkage, a subscript “o” represents a phosphodiester internucleoside linkage, a subscript “z” represents an internucleoside linkage of formula IX, which is a mesyl phosphoramidate linkage. Subscripts of nucleotides having a phosphoramidate internucleoside linkage of generic Formula XVII are bold and underlined. A superscript “m” before a C represents a 5-methyl Cytosine.

TABLE 24
Design of sense strand modified oligonucleotides targeted to
human/mouse HRPT1 having mesyl phosphoramidate linkages

| Compound | | SEQ ID |
|---|---|---|
| ID | Chemical Notation (5′ to 3′) | NO: |
| 1151789 | U_{fo}C_{yo}C_{fo}U_{yo}A_{fo}U_{yo}G_{fo}A_{yo}C_{fo}U_{yo}G_{fo}U_{yo}A_{fo}G_{yo}A_{fo}U_{yo}U_{fo}U_{yo}U_{fo}A_{yo}U_{fo}[3′- | 23 |
| | amino C7 Tag] | |
| 1405429 | U_{fo}C_{yo}C_{fo}U_{yo}A_{fo}U_{yo}G_{fo}A_{yo}C_{fo}U_{yo}G_{fo}U_{yo}A_{fo}G_{yo}A_{fo}U_{yo}U_{fo}U_{yo}U_{fz}A_{yz}U_{f} | 23 |
| 1405430 | U_{fz}C_{yz}C_{fo}U_{yo}A_{fo}U_{yo}G_{fo}A_{yo}C_{fo}U_{yo}G_{fo}U_{yo}A_{fo}G_{yo}A_{fo}U_{yo}U_{fo}U_{yo}U_{fo}A_{yo}U_{f} | 23 |
| 1405431 | U_{fz}C_{yz}C_{fo}U_{yo}A_{fo}U_{yo}G_{fo}A_{yo}C_{fo}U_{yo}G_{fo}U_{yo}A_{fo}G_{yo}A_{fo}U_{yo}U_{fo}U_{yo}U_{fz}A_{yz}U_{f} | 23 |

A subscript “f” represents a 2′-F modified nucleoside, a subscript “y” represents a 2′-OMe modified nucleoside, a subscript “s” indicates a phosphorothioate internucleoside linkage, a subscript “o” represents a phosphodiester internucleoside linkage, a subscript “z” represents an internucleoside linkage of formula IX, which is a mesyl phosphoramidate linkage. Subscripts of nucleotides having a phosphoramidate internucleoside linkage of generic Formula XVII are bold and underlined. A superscript “m” before a C represents a 5-methyl Cytosine.

### Activity Assay

Activity of various siRNA formed by annealing one antisense strand and one sense strand described above was tested in HeLa cells. HeLa cells were transfected with 6 μL/mL of siRNA using RNAiMAX for 5 hours. RNA was isolated and RNA expression was analyzed via RT-qPCR using primer probe set Hs02800695_m1(ThennoFisher). Incorporation of mesyl phosphoramidate linkages into the 3′ end of the antisense strand and into either or both the 3′ and 5′ ends of the sense strand of siRNA does not lead to a reduction in activity.

TABLE 25
Activity of siRNAs having mesyl phosphoramidate linkages
against human HPRT1

| | | Linkage mod. | Linkage mod. | |
|---|---|---|---|---|
| Antisense | Sense | position in | position in | IC50 |
| Strand | Strand | antisense strand | sense strand | (nM) |
| 1073762 | 1151789 | n/a | n/a | 0.091 |
| 1405420 | 1405429 | 3′ | 3′ | 0.085 |
| 1405420 | 1405430 | 3′ | 5′ | 0.065 |
| 1405420 | 1405431 | 3′ | 3′ and 5′ | 0.066 |
| 1405427 | 1405429 | 5′ | 3′ | 0.457 |
| 1405427 | 1405430 | 5′ | 5′ | 0.831 |
| 1405427 | 1405431 | 5′ | 3′ and 5′ | 1.391 |
| 1405428 | 1405429 | 3′ and 5′ | 3′ | 1.19 |
| 1405428 | 1405430 | 3′ and 5′ | 5′ | 0.816 |
| 1405428 | 1405431 | 3′ and 5′ | 3′ and 5′ | 0.934 |

### Example 13: Design and Activity of siRNA to Human/Mouse PTEN Having Mesyl Phosphoramidate Internucleoside Linkages In Vitro

siRNA

Single-stranded siRNA and double-stranded siRNA comprising modified oligonucleotides having mesyl phosphoramidate internucleoside linkages (Formula IX) in the antisense strands were synthesized and tested. Each internucleoside linkage is either a phosphorothioate internucleoside linkage (“s”), a phosphodiester internucleoside linkage (“o”), or a mesyl phosphoramidate internucleoside linkage (“z”), indicated by formula IX below.

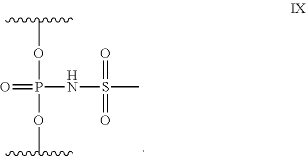 [SMILES: *OP(=O)(NS(C)(=O)=O)O*]

Each antisense strand has the sequence TUAUCUAUAAUGAUCAGGUAA (SEQ ID NO: 24) and has three mismatches to PTEN cDNA, the cDNA of ENSEMBL Accession No. ENST00000371953.8 from ENSEMBL version 99: January 2020, human reference assembly version GRCh38.p13 located on the forward strand of chromosome 10 (CM000682.2) from positions 87,863,625 to 87,971,930 (SEQ ID NO: 26) from 1962 to 1982, and each antisense strand has a 5′-phosphate. For double-stranded siRNA, the sense strand 790973 has the sequence ACCUGAUCAUUAUAGAUAA (SEQ ID NO: 25) and has one mismatch to the cDNA of ENSEMBL Accession No. ENST00000371953.8 from ENSEMBL version 99: January 2020, human reference assembly version GRCh38.p13 located on the forward strand of chromosome 10 (CM000682.2) from positions 87,863,625 to 87,971,930 (SEQ ID NO: 26) from 1964 to 1982. Each internucleoside linkage of the sense strand is either a phosphodiester internucleoside linkage (“o”) or a phosphorothioate internucleoside linkage (“s”), and the sense strand has the chemical notation (5′ to 3′): A_{fs}C_{ys}C_{fo}U_{yo}G_{fo}A_{yo}U_{fo}C_{yo}A_{fo}U_{yo}U_{fo}A_{yo}U_{fo}A_{yo}G_{fo}A_{yo}U_{fs}A_{ys}A_{f} (SEQ ID ON: 25).

TABLE 26
Design of antisense strand modified oligonucleotides targeted
to PTEN having mesyl phosphoramidate linkages

| Compound | | SEQ ID |
|---|---|---|
| ID | Chemical Notation (5′ to 3′) | NO: |
| 456963 | p.TₑₛU_{fs}A_{yo}U_{fs}C_{yo}U_{fs}A_{yo}U_{fs}A_{yo}A_{fs}U_{yo}G_{fs}A_{yo}U_{fs}C_{ys}A_{fs}G_{ys}G_{fs}U_{ys}AₑₛAₑ | 24 |
| 1421366 | p.T_{ez}U_{fz}A_{yo}U_{fs}C_{yo}U_{fs}A_{yo}U_{fs}A_{yo}A_{fs}U_{yo}G_{fs}A_{yo}U_{fs}C_{ys}A_{fs}G_{ys}G_{fs}U_{ys}AₑₛAₑ | 24 |
| 1439694 | p.TₑₛU_{fs}A_{yz}U_{fz}C_{yo}U_{fs}A_{yo}U_{fs}A_{yo}A_{fs}U_{yo}G_{fs}A_{yo}U_{fs}C_{ys}A_{fs}G_{ys}G_{fs}U_{ys}AₑₛAₑ | 24 |
| 1440988 | p.TₑₛU_{fs}A_{yo}U_{fs}C_{yz}U_{fz}A_{yo}U_{fs}A_{yo}A_{fs}U_{yo}G_{fs}A_{yo}U_{fs}C_{ys}A_{fs}G_{ys}G_{fs}U_{ys}AₑₛAₑ | 24 |
| 1440992 | p.TₑₛU_{fs}A_{yo}U_{fs}C_{yo}U_{fs}A_{yz}U_{fz}A_{yo}A_{fs}U_{yo}G_{fs}A_{yo}U_{fs}C_{ys}A_{fs}G_{ys}G_{fs}U_{ys}AₑₛAₑ | 24 |
| 1440993 | p.TₑₛU_{fs}A_{yo}U_{fs}C_{yo}U_{fs}A_{yo}U_{fs}A_{yz}A_{fz}U_{yo}G_{fs}A_{yo}U_{fs}C_{ys}A_{fs}G_{ys}G_{fs}U_{ys}AₑₛAₑ | 24 |
| 1440994 | p.TₑₛU_{fs}A_{yo}U_{fs}C_{yo}U_{fs}A_{yo}U_{fs}A_{yo}A_{fs}U_{yz}G_{fz}A_{yo}U_{fs}C_{ys}A_{fs}G_{ys}G_{fs}U_{ys}AₑₛAₑ | 24 |
| 1440995 | p.TₑₛU_{fs}A_{yo}U_{fs}C_{yo}U_{fs}A_{yo}U_{fs}A_{yo}A_{fs}U_{yo}G_{fs}A_{yz}U_{fz}C_{ys}A_{fs}G_{ys}G_{fs}U_{ys}AₑₛAₑ | 24 |
| 1441021 | p.TₑₛU_{fs}A_{yo}U_{fs}C_{yo}U_{fs}A_{yo}U_{fs}A_{yo}A_{fs}U_{yo}G_{fs}A_{yo}U_{fs}C_{yz}A_{fz}G_{ys}G_{fs}U_{ys}AₑₛAₑ | 24 |
| 1441022 | p.TₑₛU_{fs}A_{yo}U_{fs}C_{yo}U_{fs}A_{yo}U_{fs}A_{yo}A_{fs}U_{yo}G_{fs}A_{yo}U_{fs}C_{ys}A_{fs}G_{yz}G_{fz}U_{ys}AₑₛAₑ | 24 |
| 1441023 | p.TₑₛU_{fs}A_{yo}U_{fs}C_{yo}U_{fs}A_{yo}U_{fs}A_{yo}A_{fs}U_{yo}G_{fs}A_{yo}U_{fs}C_{ys}A_{fs}G_{ys}G_{fs}U_{yz}A_{ez}Aₑ | 24 |

A “p.” represents a 5′-phosphate. A subscript “f” represents a 2′-F modified nucleoside, a subscript “y” represents a 2′-OMe modified nucleoside, a subscript “s” indicates a phosphorothioate internucleoside linkage, a subscript “o” represents a phosphodiester internucleoside linkage, a subscript “z” represents an internucleoside linkage of formula IX, which is a mesyl phosphoramidate linkage. Subscripts of nucleotides having a phosphoramidate internucleoside linkage of generic Formula XVII are bold and underlined. A superscript “m” before a C represents a 5-methyl Cytosine.

### Activity Assay

Activity of various siRNA formed by annealing one antisense strand and one sense strand described above was tested in HeLa cells. HeLa cells were transfected with 6 μL/mL of siRNA using RNAIMAX for 6 hours. RNA was isolated and RNA expression was analyzed via RT-qPCR using primer probe set Hs02800695_m1(ThennoFisher).

TABLE 27
Activity of double-stranded siRNAs having mesyl
phosphoramidate linkages against human PTEN

| | | Linkage mod. | |
|---|---|---|---|
| Antisense | Sense | position in | IC50 |
| Strand | Strand | antisense strand | (nM) |
| 456963 | 790973 | N/A | 0.98 |
| 1421366 | 790973 | Positions 1-2, | 2.87 |
| | | 2-3 | |
| 1439694 | 790973 | Positions 3-4, | 2.89 |
| | | 4-5 | |
| 1440988 | 790973 | Positions 5-6, | >10 |
| | | 6-7 | |
| 1440992 | 790973 | Positions 7-8, | 2.17 |
| | | 8-9 | |
| 1440993 | 790973 | Positions 9-10, | 0.84 |
| | | 10-11 | |
| 1440994 | 790973 | Positions 11-12, | >10 |
| | | 12-13 | |
| 1440995 | 790973 | Positions 13-14, | |
| | | 14-15 | 10.00 |
| 1441021 | 790973 | Positions 15-16, | |
| | | 16-17 | 10.00 |
| 1441022 | 790973 | Positions 17-18, | |
| | | 18-19 | 1.24 |
| 1441023 | 790973 | Positions 19-20, | |
| | | 20-21 | 2.41 |

### Example 14: Design of siRNA to SOD-1 Having a C16 Modified-Phosphoramidate Internucleoside Linkage

Double-stranded siRNA comprising a modified oligonucleotide having a mesyl phosphoramidate internucleoside linkages of Formula XIX in the sense strand was synthesized and tested in vitro.

For the sense strand, each internucleoside linkage is either a phosphorothioate internucleoside linkage (“s”), a phosphodiester internucleoside linkage (“o”), or a modified phosphoramidate internucleoside linkage (“XIX”), as shown below.

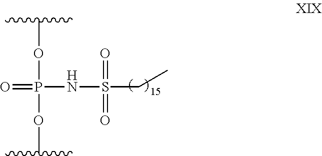

The sense strand has the chemical notation (5′ to 3′):
C_{ys}A_{ys}U_{ys}U_{ys}U_{ys}U_{yXIX}A_{fo}A_{yo}U_{fo}C_{fo}C_{fo}U_{yo}C_{yo}A_{yo}C_{yo}U_{yo}C_{yo}U_{yo}A_{ys}A_{ys}A_{y}, (SEQ ID NO: 30) wherein a subscript “f” represents a 2′-F modified nucleoside, a subscript “y” represents a 2′-OMe modified nucleoside, a subscript “s” indicates a phosphorothioate internucleoside linkage, a subscript “o” represents a phosphodiester internucleoside linkage, and a subscript XIX represents an internucleoside linkage of Formula XIX.

The antisense strand has a 5′-vinyl phosphonate (vP). Each internucleoside linkage of the antisense strand is either a phosphodiester internucleoside linkage (“o”) or a phosphorothioate internucleoside linkage (“s”). The antisense strand has the chemical notation (5′ to 3′):

vP-U_{yo}U_{fo}U_{yo}A_{yo}G_{yo}A_{fo}G_{yo}U_{fo}G_{fo}A_{yo}G_{yo}G_{yo}A_{yo}U_{fo}U_{yo}A_{fo}A_{yo}A_{yo}A_{yo}U_{yo}G_{ys}A_{ys}A_{y} (SEQ ID NO: 31), wherein a subscript “f” represents a 2′-F modified nucleoside, a subscript “y” represents a 2′-OMe modified nucleoside, a subscript “s” indicates a phosphorothioate internucleoside linkage and a subscript “o” represents a phosphodiester internucleoside linkage.

### Example 15: Tolerability of Modified Oligonucleotides Having Mesyl Phosphoramidate Internucleoside Linkages In Vivo in Wild-Type Mice

### Modified Oligonucleotides

Modified oligonucleotides comprising multiple mesyl phosphoramidate internucleoside linkages (Formula IX) were synthesized and tested. The modified oligonucleotides are each 5-10-5 MOE gapmers with a sugar motif of: eeeeeddddddddddeeeee where “e” represents a 2′-MOE modified sugar moiety, and “d” represents a β-D-2′-deoxyribosyl sugar moiety. The modified oligonucleotides are 100% complementary to human MAPT, GENIBANK accession number NT_010783.15 truncated from 9240000 to 9381000 (SEQ TD NO: 32).

Each internucleoside linkage is a phosphorothioate intemucleoside linkage (“s”), a phosphodiester internucleoside linkage (“o”) or a mesyl phosphoramidate internucleoside linkage (“z”), as indicated in the table below.

Oligonucleotides described above were tested in wild-type female C57/B16 mice to assess the tolerability of the oligonucleotides. Wild-type female C57/B16 mice each received a single ICV dose of 700 μg of modified oligonucleotide listed in the table below. Each treatment group consisted of 2 mice. A group of 2 mice received PB S as a negative control. At 3 hours post-injection, mice were evaluated according to seven different criteria. The criteria are (1) the mouse was bright, alert, and responsive; (2) the mouse was standing or hunched without stimuli; (3) the mouse showed any movement without stimuli; (4) the mouse demonstrated forward movement after it was lifted; (5) the mouse demonstrated any movement after it was lifted; (6) the mouse responded to tail pinching; (7) regular breathing. For each of the 7 criteria, a mouse was given a subscore of 0 if it met the criteria and 1 if it did not (the functional observational battery score or FOB). After all 7 criteria were evaluated, the scores were summed for each mouse. The results are presented in the table below. Oligonucleotides comprising mesyl phosphoramidate intemucleoside linkages have similar tolerability in the mouse CNS as the parent oligonucleotide. Oligonucleotides having mesyl terminal phosphoramidate linkages as well as two mesyl phosphoramidate linkages in the deoxy region have improved CNS tolerability.

TABLE 28
CNS Tolerability of modified oligonucleotides containing
mesyl phosphoramidate linkages

| Compound | | SEQ ID | 3 hour |
|---|---|---|---|
| ID | Chemical Notation (5′ to 3′) | NO: | FOB |
| 613039 | TₑₛGₑₒ^{m}CₑₒAₑₒTₑₒG_{ds}G_{ds}T_{ds}G_{ds}T_{ds}A_{ds}G_{ds}^{m}C_{ds}^{m}C_{ds}^{m}C_{ds}^{m}Cₑₒ^{m}Cₑₒ^{m}CₑₛTₑₛGₑ | 27 | 6, 6 |
| 1405498 | TₑₛGₑₒ^{m}CₑₒAₑₒTₑₒG_{dz}G_{dz}T_{ds}G_{ds}T_{ds}A_{ds}G_{ds}^{m}C_{ds}^{m}C_{ds}^{m}C_{ds}^{m}Cₑₒ^{m}Cₑₒ^{m}CₑₛTₑₛGₑ | 27 | 6, 6 |
| 1405499 | TₑₛGₑₒ^{m}CₑₒAₑₒTₑₒG_{ds}G_{dz}T_{dz}G_{ds}T_{ds}A_{ds}G_{ds}^{m}C_{ds}^{m}C_{ds}^{m}C_{ds}^{m}Cₑₒ^{m}Cₑₒ^{m}CₑₛTₑₛGₑ | 27 | 6, 6 |
| 1405500 | TₑₛGₑₒ^{m}CₑₒAₑₒTₑₒG_{ds}G_{ds}T_{dz}G_{dz}T_{ds}A_{ds}G_{ds}^{m}C_{ds}^{m}C_{ds}^{m}C_{ds}^{m}Cₑₒ^{m}Cₑₒ^{m}CₑₛTₑₛGₑ | 27 | 6, 7 |
| 1405501 | TₑₛGₑₒ^{m}CₑₒAₑₒTₑₒG_{ds}G_{ds}T_{ds}G_{ds}T_{ds}A_{ds}G_{dz}^{m}C_{dz}^{m}C_{ds}^{m}C_{ds}^{m}Cₑₒ^{m}Cₑₒ^{m}CₑₛTₑₛGₑ | 27 | 5, 5 |
| 1421514 | T_{ez}Gₑₒ^{m}CₑₒAₑₒTₑₒG_{ds}G_{ds}T_{ds}G_{ds}T_{ds}A_{ds}G_{ds}^{m}C_{ds}^{m}C_{ds}^{m}C_{ds}^{m}Cₑₒ^{m}Cₑₒ^{m}C_{ez}T_{ez}Gₑ | 27 | 6, 5 |
| 1421517 | T_{ez}Gₑₒ^{m}CₑₒAₑₒTₑₒG_{ds}G_{dz}T_{dz}G_{ds}T_{ds}A_{ds}G_{ds}^{m}C_{ds}^{m}C_{ds}^{m}C_{ds}^{m}Cₑₒ^{m}Cₑₒ^{m}C_{ez}T_{ez}Gₑ | 27 | 4, 4 |
| 613369 | GₑₛTₑₒTₑₒTₑₒTₑₒ^{m}C_{ds}A_{ds}A_{ds}A_{ds}^{m}C_{ds}A_{ds}^{m}C_{ds}A_{ds}^{m}C_{ds}^{m}C_{ds}TₑₒTₑₒ^{m}CₑₛAₑₛTₑ | 28 | 0, 0 |
| 1405502 | GₑₛTₑₒTₑₒTₑₒTₑₒ^{m}C_{dz}A_{dz}A_{ds}A_{ds}^{m}C_{ds}A_{ds}^{m}C_{ds}A_{ds}^{m}C_{ds}^{m}C_{ds}TₑₒTₑₒ^{m}CₑₛAₑₛTₑ | 28 | 0, 0 |
| 1405503 | GₑₛTₑₒTₑₒTₑₒTₑₒ^{m}C_{ds}A_{dz}A_{dz}A_{ds}^{m}C_{ds}A_{ds}^{m}C_{ds}A_{ds}^{m}C_{ds}^{m}C_{ds}TₑₒTₑₒ^{m}CₑₛAₑₛTₑ | 28 | 1, 1 |
| 1405504 | GₑₛTₑₒTₑₒTₑₒTₑₒ^{m}C_{ds}A_{ds}A_{dz}A_{dz}^{m}C_{ds}A_{ds}^{m}C_{ds}A_{ds}^{m}C_{ds}^{m}C_{ds}TₑₒTₑₒ^{m}CₑₛAₑₛTₑ | 28 | 1, 1 |
| 1405505 | GₑₛTₑₒTₑₒTₑₒTₑₒ^{m}C_{ds}A_{ds}A_{ds}A_{ds}^{m}C_{ds}A_{ds}^{m}C_{dz}A_{dz}^{m}C_{ds}^{m}C_{ds}TₑₒTₑₒ^{m}CₑₛAₑₛTₑ | 28 | 2, 2 |
| 1421518 | G_{ez}TₑₒTₑₒTₑₒTₑₒ^{m}C_{ds}A_{ds}A_{ds}A_{ds}^{m}C_{ds}A_{ds}^{m}C_{ds}A_{ds}^{m}C_{ds}^{m}C_{ds}TₑₒTₑₒ^{m}C_{ez}A_{ez}Tₑ | 28 | 0, 0 |
| 1421519 | G_{ez}TₑₒTₑₒTₑₒTₑₒ^{m}C_{ds}A_{dz}A_{dz}A_{ds}^{m}C_{ds}A_{ds}^{m}C_{ds}A_{ds}^{m}C_{ds}^{m}C_{ds}TₑₒTₑₒ^{m}C_{ez}A_{ez}Tₑ | 28 | 1, 1 |

A subscript “o” indicates a phosphodiester internucleoside linkage, a subscript “s” indicates a phosphorothioate internucleoside linkage, a subscript “z” represents an internucleoside linkage of formula IX, which is a mesyl phosphoramidate linkage. Subscripts of nucletides having a phosphoramidate internucleoside linkage of generic Formula XVII are bold and underlined. A superscript “m” before a C represents a 5-methyl Cytosine.

### Example 16: Design, Activity and Tolerability of Modified Oligonucleotides Having Various Mesyl Phosphoramidate Internucleoside Linkages In Vitro

### Modified Oligonucleotides

Modified oligonucleotides comprising multiple mesyl phosphoramidate internucleoside linkages (Formula IX), phosphorothioate internucleoside linkages, and phosphodiester internucleoside linkages, were synthesized and tested. The modified oligonucleotides are each 3-10-3 cEt gapmers with a sugar motif of: kkkddddddddddkkk (a 3-10-3 cEt motif) where “k” represents a cEt modified sugar moiety, and “d” represents a β-D-2′-deoxyribosyl sugar moiety. Each of the modified oligonucleotides has the same nucleobase sequence, GCATGTTCTCACATTA (SEQ ID NO: 5), which is 100% complementary to mouse CXCL12, GENBANK NT_039353.7 truncated from 69/430,515 to 69/445,350 (SEQ ID NO: 1), at position 6877 to 6892. Each internucleoside linkage is either a phosphorothioate internucleoside linkage (“s”), a phosphodiester internucleoside linkage (“o”), or a mesyl phosphoramidate internucleoside linkage (“z”).

### In Vitro Assays

In vitro activity of modified oligonucleotides described above was determined as described in Example 1. In vitro toxicity of modified oligonucleotides described above was determined as described in Example 3. The internucleoside linkages of the modified nucleosides are indicated in subscripts in the table below.

TABLE 29
Design, Activity, and Tolerability of modified oligonucleotides having
multiple mesyl phosphoramidates linkages complementary to CXCL12

| | | CXCL12 | Caspase | |
|---|---|---|---|---|
| Compound | | IC₅₀ | Activation | SEQ ID |
| Number | Chemistry Notation (5′-3′) | (nM) | (% Mock) | NO. |
| 558807 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 101 | 1223 | 5 |
| 1193271 | Gₖₒ^{m}CₖₒAₖₒT_{ds}G_{ds}T_{ds}T_{ds}^{m}C_{ds}T_{ds}^{m}C_{ds}A_{ds}^{m}C_{ds}A_{ds}TₖₒTₖₒAₖ | 87 | 1966 | 5 |
| 1233817 | Gₖₛ^{m}CₖₒAₖₒT_{ds}G_{ds}T_{ds}T_{ds}^{m}C_{ds}T_{ds}^{m}C_{ds}A_{ds}^{m}C_{ds}A_{ds}TₖₒTₖₛAₖ | 58 | 1712 | 5 |
| 1467836 | G_{kz}^{m}CₖₒAₖₒT_{ds}G_{ds}T_{ds}T_{ds}^{m}C_{ds}T_{ds}^{m}C_{ds}A_{ds}^{m}C_{ds}A_{ds}TₖₒT_{kz}Aₖ | 100 | 1595 | 5 |
| 1467198 | G_{kz}^{m}CₖₒAₖₒT_{ds}G_{ds}T_{ds}T_{ds}^{m}C_{ds}T_{ds}^{m}C_{ds}A_{ds}^{m}C_{ds}A_{do}TₖₒT_{kz}Aₖ | 77 | 1733 | 5 |
| 1467199 | G_{kz}^{m}CₖₒA_{kz}T_{ds}G_{ds}T_{ds}T_{ds}^{m}C_{ds}T_{ds}^{m}C_{ds}A_{ds}^{m}C_{ds}A_{dz}TₖₒT_{kz}Aₖ | 63 | 1462 | 5 |
| 1467837 | Gₖₒ^{m}CₖₒA_{kz}T_{ds}G_{ds}T_{ds}T_{ds}^{m}C_{ds}T_{ds}^{m}C_{ds}A_{ds}^{m}C_{ds}A_{dz}TₖₒTₖₒAₖ | 72 | 1809 | 5 |
| 1467838 | G_{kz}^{m}CₖₒAₖₒT_{ds}G_{dz}T_{dz}T_{ds}^{m}C_{ds}T_{ds}^{m}C_{ds}A_{ds}^{m}C_{ds}A_{ds}TₖₒT_{kz}Aₖ | 110 | 693 | 5 |
| 1467839 | G_{kz}^{m}CₖₒAₖₛT_{ds}G_{dz}T_{dz}T_{ds}^{m}C_{ds}T_{ds}^{m}C_{ds}A_{ds}^{m}C_{ds}A_{ds}TₖₒT_{kz}Aₖ | 208 | 502 | 5 |
| 1467821 | G_{kz}^{m}CₖₒAₖₛT_{ds}G_{dz}T_{dz}T_{ds}^{m}C_{ds}T_{ds}^{m}C_{ds}A_{ds}^{m}C_{ds}A_{ds}TₖₒT_{kz}Aₖ | 172 | 495 | 5 |
| 1467840 | G_{kz}^{m}CₖₒAₖₒT_{dz}G_{dz}T_{dz}T_{dz}^{m}C_{ds}T_{ds}^{m}C_{ds}A_{ds}^{m}C_{ds}A_{dz}TₖₒT_{kz}Aₖ | 152 | 654 | 5 |
| 1467841 | G_{kz}^{m}CₖₒAₖₒT_{ds}G_{ds}T_{ds}T_{ds}^{m}C_{ds}T_{ds}^{m}C_{dz}A_{dz}^{m}C_{dz}A_{dz}TₖₒT_{kz}Aₖ | 132 | 1216 | 5 |

A subscript “k” represents a cEt nucleoside, a subscript “d” represents a stereostandard DNA nucleoside, a subscript “s” indicates a phosphorothioate internucleoside linkage, a subscript “o” indicates a phosphodiester internucleoside linkage, and a subscript “z” represents an internucleoside linkage of formula IX, which is a mesyl phosphoramidate linkage. Subscripts of nucleotides having an internucleoside linkage of formula IX are bold and underlined. A superscript “m” before a C represents a 5-methyl Cytosine.

### Example 17: Activity and Tolerability of Modified Oligonucleotides with Mesyl Phosphoramidate Internucleoside Linkages In Vivo

### Modified Oligonucleotides

GalNAc-conjugated modified oligonucleotides comprising multiple mesyl phosphoramidate internucleoside linkages (Formula IX) and phosphorothioate internucleoside linkages were synthesized and tested. The modified oligonucleotides are each 3-10-3 cEt gapmers with a sugar motif of: kkkddddddddddkkk (a 3-10-3 cEt motif) where “k” represents a cEt modified sugar moiety, and “d” represents a β-D-2′-deoxyribosyl sugar moiety. Each of the modified oligonucleotides has the same nucleobase sequence, GCATGTTCTCACATTA (SEQ ID NO: 5), which is 100% complementary to mouse CXCL12, GENBANK NT_039353.7 truncated from 69/430,515 to 69/445,350 (SEQ ID NO: 1), at position 6877 to 6892. Each internucleoside linkage is either a phosphorothioate internucleoside linkage (“s”), a phosphodiester internucleoside linkage (“o”), or a mesyl phosphoramidate internucleoside linkage (“z”).

The GalNAc moiety is conjugated to the 5′ oxygen of the oligonucleotide via a THA linker, as shown below:

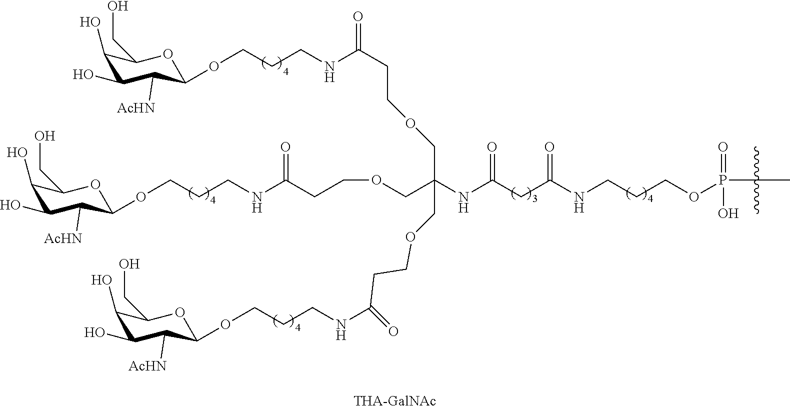

In addition to compounds containing a mesyl phosphoramidate internucleoside linkage, a 3′-GalNAc conjugated version of Compound No. 936053 (1306456) was tested. This compound has the sequence GCATGTTCTCACATTA (SEQ ID NO: 5) and a sugar motif of kkk-d-m-dddddddd-kkk, wherein each “k” represents a cEt nucleoside, each “d” represents a stereo-standard DNA nucleoside, and “m” represents a 2′-OMe nucleoside. It has a GalNAc conjugated at the 3′- oxygen of the oligonucleotide via a THA linker as shown above.

Compound No. 936053 was described in WO2019/157531. It was chosen as the parent of the comparator compound because it has reduced toxicity relative to 558807 as well as reduced potency in vivo. Note that at least some of the observed potency of 558807 is “false”; that is, the RNA reduction observed is not specific to RNAse H mediated reduction of CXCL 12 RNA, but rather, is related to global reductions in RNA due to cellular toxicity. Therefore, Compound No. 936503 represents a better comparator compound for determining the relative in vivo potency of compounds comprising mesyl phosphoramidate internucleoside linkages.

TABLE 30

| Compound | | SEQ ID |
|---|---|---|
| Number | Chemistry Notation (5′-3′) | NO. |
| 1306456 | Gₖₛ^{m}CₖₛAₖₛT_{ds}G_{ys}T_{ds}T_{ds}^{m}C_{ds} | 5 |
| | T_{ds}^{m}C_{ds}A_{ds}^{m}C_{ds}A_{ds}TₖₛTₖₛAₖ- | |
| | THA-GalNAc | |
| 1462752 | THA-GalNAc- | 5 |
| | Gₖₛ^{m}CₖₛAₖₛT_{dz}G_{dz}T_{dz}T_{ds}^{m}C_{ds} | |
| | T_{ds}^{m}C_{ds}A_{ds}^{m}C_{ds}A_{ds}TₖₛTₖₛAₖ | |
| 1462753 | THA-GalNAc- | 5 |
| | Gₖₛ^{m}CₖₛAₖₛT_{dz}G_{dz}T_{dz}T_{dz}^{m}C_{ds} | |
| | T_{ds}^{m}C_{ds}A_{ds}^{m}C_{ds}A_{ds}TₖₛTₖₛAₖ | |
| 1462754 | THA-GalNAc- | 5 |
| | Gₖₛ^{m}CₖₛAₖₛT_{dz}G_{dz}T_{dz}T_{dz}^{m}C_{dz} | |
| | T_{ds}^{m}C_{ds}A_{ds}^{m}C_{ds}A_{ds}TₖₛTₖₛAₖ | |

### Study Design

For the in vivo activity and tolerability study in the tables below, 3 BALB/C mice per group were administered modified oligonucleotide by subcutaneous injection and sacrificed after 72 hours. Modified oligonucleotides were dosed at 0.2, 0.6, 1.8, 5.4, or 15 mg/kg.

Tissue were collected and mRNA was isolated and levels of CXCL12 in liver samples were measured by RT-qPCR with primer probe set RTS2605 as described above. Plasma ALT was measured. Elevations in ALT are associated with liver toxicity.

Expression levels were normalized with Ribogreen® and are presented relative to levels in mice treated with PBS.

TABLE 31
In Vivo Activity and Toxicity of modified
oligonucleotides complementary to CXCL12

| | in vivo | | |
|---|---|---|---|
| | CXCL12 | ALT | |
| | ED50 | @5.4 | ALT @ |
| Compound | liver | mg/kg | 15 mg/kg |
| ID | (mg/kg) | (IU/L) | (IU/L) |
| 1306456 | 0.39 | 29 | 34 |
| 1462752 | 0.17 | 55 | 336 |
| 1462753 | 0.17 | 32 | 107 |
| 1462754 | 0.30 | 34 | 79 |

### Example 18: Design, Activity and Tolerability of Modified Oligonucleotides Having Various Mesyl Phosphoramidate, Phosphorothioate, and Phosphodiester Internucleoside Linkages In Vitro

### Modified Oligonucleotides

Modified oligonucleotides comprising multiple mesyl phosphoramidate internucleoside linkages (Formula IX), phosphorothioate internucleoside linkages, and phosphodiester internucleoside linkages, were synthesized and tested. The modified oligonucleotides are each 3-10-3 cEt gapmers with a sugar motif of: kkkddddddddddkkk (a 3-10-3 cEt motif) where “k” represents a cEt modified sugar moiety, and “d” represents a β-D-2′-deoxyribosyl sugar moiety. Each of the modified oligonucleotides has the same nucleobase sequence, GCATGTTCTCACATTA (SEQ ID NO: 5), which is 100% complementary to mouse CXCL12, GENBANK NT_039353.7 truncated from 69/430,515 to 69/445,350 (SEQ ID NO: 1), at position 6877 to 6892. Each internucleoside linkage is either a phosphorothioate internucleoside linkage (“s”), a phosphodiester internucleoside linkage (“o”), or a mesyl phosphoramidate internucleoside linkage “z”).

### In Vitro Assays

In vitro activity of modified oligonucleotides described above was determined at 100 nM only, and the results are presented as the % expression relative to untreated control cells. For selected compounds, in vitro activity dose response was tested as described in Example 1. In vitro toxicity of modified oligonucleotides described above was determined as described in Example 3. The intemucleoside linkages of the modified nucleosides are indicated in subscripts in the table below.

TABLE 32
Design, Activity, and Tolerability of modified oligonucleotides having
multiple mesyl phosphoramidates linkages complementary to CXCL12

| | | CXCL12 | | | |
|---|---|---|---|---|---|
| | | at | CXCL12 | Caspase | SEQ |
| Compound | | 100 nM | IC₅₀ | Activation | ID |
| Number | Chemistry Notation (5′-3′) | (% UTC) | (nM) | (% Mock) | NO. |
| 558807 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 34 | 88 | 1287 | 5 |
| 1467797 | Gₖₛ^{m}CₖₛAₖₛT_{ds}G_{ds}T_{dz}T_{dz}^{m}C_{dz}T_{ds}^{m}C_{ds}A_{ds}^{m}C_{ds}A_{ds}TₖₛTₖₛAₖ | 45 | 170 | 393 | 5 |
| 1467798 | Gₖₛ^{m}CₖₛAₖₛT_{ds}G_{ds}T_{ds}T_{dz}^{m}C_{dz}T_{dz}^{m}C_{ds}A_{ds}^{m}C_{ds}A_{ds}TₖₛTₖₛAₖ | 70 | n.d. | 974 | 5 |
| 1467799 | Gₖₛ^{m}CₖₛAₖₛT_{ds}G_{ds}T_{ds}T_{ds}^{m}C_{dz}T_{dz}^{m}C_{dz}A_{ds}^{m}C_{ds}A_{ds}TₖₛTₖₛAₖ | 73 | n.d. | 1023 | 5 |
| 1467800 | Gₖₛ^{m}CₖₛAₖₛT_{ds}G_{ds}T_{ds}T_{ds}^{m}C_{ds}T_{dz}^{m}C_{dz}A_{dz}^{m}C_{ds}A_{ds}TₖₛTₖₛAₖ | 59 | n.d. | 939 | 5 |
| 1467801 | Gₖₛ^{m}CₖₛAₖₛT_{ds}G_{ds}T_{ds}T_{ds}^{m}C_{ds}T_{ds}^{m}C_{dz}A_{dz}^{m}C_{dz}A_{ds}TₖₛTₖₛAₖ | 53 | n.d. | 682 | 5 |
| 1467802 | Gₖₛ^{m}CₖₛAₖₛT_{ds}G_{dz}T_{dz}T_{dz}^{m}C_{dz}T_{ds}^{m}C_{ds}A_{ds}^{m}C_{ds}A_{ds}TₖₛTₖₛAₖ | 52 | 199 | 269 | 5 |
| 1467803 | Gₖₛ^{m}CₖₛAₖₛT_{ds}G_{ds}T_{dz}T_{dz}^{m}C_{dz}T_{dz}^{m}C_{ds}A_{ds}^{m}C_{ds}A_{ds}TₖₛTₖₛAₖ | 75 | n.d. | 792 | 5 |
| 1467804 | Gₖₛ^{m}CₖₛAₖₛT_{ds}G_{ds}T_{ds}T_{dz}^{m}C_{dz}T_{dz}^{m}C_{dz}A_{ds}^{m}C_{ds}A_{ds}TₖₛTₖₛAₖ | 60 | n.d. | 983 | 5 |
| 1467805 | Gₖₛ^{m}CₖₛAₖₛT_{ds}G_{ds}T_{ds}T_{ds}^{m}C_{dz}T_{dz}^{m}C_{dz}A_{dz}^{m}C_{ds}A_{ds}TₖₛTₖₛAₖ | 50 | n.d. | 1247 | 5 |
| 1467806 | Gₖₛ^{m}CₖₛAₖₛT_{ds}G_{ds}T_{ds}T_{ds}^{m}C_{ds}T_{dz}^{m}C_{dz}A_{dz}^{m}C_{dz}A_{ds}TₖₛTₖₛAₖ | 50 | n.d. | 1062 | 5 |
| 1467807 | Gₖₛ^{m}CₖₛAₖₛT_{dz}G_{dz}T_{ds}T_{ds}^{m}C_{ds}T_{ds}^{m}C_{ds}A_{ds}^{m}C_{dz}A_{dz}TₖₛTₖₛAₖ | 63 | n.d. | 443 | 5 |
| 1467808 | Gₖₛ^{m}CₖₛAₖₛT_{dz}G_{dz}T_{ds}T_{ds}^{m}C_{ds}T_{ds}^{m}C_{ds}A_{dz}^{m}C_{dz}A_{ds}TₖₛTₖₛAₖ | 52 | n.d. | 615 | 5 |
| 1467809 | Gₖₛ^{m}CₖₛAₖₛT_{dz}G_{dz}T_{ds}T_{ds}^{m}C_{ds}T_{ds}^{m}C_{dz}A_{dz}^{m}C_{ds}A_{ds}TₖₛTₖₛAₖ | 51 | n.d. | 408 | 5 |
| 1467810 | Gₖₛ^{m}CₖₛAₖₛT_{dz}G_{dz}T_{ds}T_{ds}^{m}C_{ds}T_{dz}^{m}C_{dz}A_{ds}^{m}C_{ds}A_{ds}TₖₛTₖₛAₖ | 57 | n.d. | 426 | 5 |
| 1467811 | Gₖₛ^{m}CₖₛAₖₛT_{dz}G_{dz}T_{ds}T_{ds}^{m}C_{dz}T_{dz}^{m}C_{ds}A_{ds}^{m}C_{ds}A_{ds}TₖₛTₖₛAₖ | 54 | n.d. | 347 | 5 |
| 1467812 | Gₖₛ^{m}CₖₛAₖₛT_{dz}G_{dz}T_{ds}T_{dz}^{m}C_{dz}T_{ds}^{m}C_{ds}A_{ds}^{m}C_{ds}A_{ds}TₖₛTₖₛAₖ | 36 | 150 | 327 | 5 |
| 1467813 | Gₖₛ^{m}CₖₛA_{kz}T_{dz}G_{ds}T_{ds}T_{ds}^{m}C_{ds}T_{ds}^{m}C_{ds}A_{ds}^{m}C_{dz}A_{dz}TₖₛTₖₛAₖ | 39 | 83 | 1236 | 5 |
| 1467814 | Gₖₛ^{m}CₖₛAₖₛT_{ds}G_{dz}T_{dz}T_{ds}^{m}C_{ds}T_{ds}^{m}C_{ds}A_{ds}^{m}C_{dz}A_{dz}TₖₛTₖₛAₖ | 78 | 220 | 241 | 5 |
| 1467815 | Gₖₛ^{m}CₖₛAₖₛT_{ds}G_{ds}T_{dz}T_{dz}^{m}C_{ds}T_{ds}^{m}C_{ds}A_{ds}^{m}C_{dz}A_{dz}TₖₛTₖₛAₖ | 59 | n.d. | 435 | 5 |
| 1467816 | Gₖₛ^{m}CₖₛAₖₛT_{ds}G_{ds}T_{ds}T_{dz}^{m}C_{dz}T_{ds}^{m}C_{ds}A_{ds}^{m}C_{dz}A_{dz}TₖₛTₖₛAₖ | 67 | n.d. | 714 | 5 |
| 1467817 | Gₖₛ^{m}CₖₛAₖₛT_{ds}G_{ds}T_{ds}T_{ds}^{m}C_{dz}T_{dz}^{m}C_{ds}A_{ds}^{m}C_{dz}A_{dz}TₖₛTₖₛAₖ | 38 | n.d. | 1146 | 5 |
| 1467818 | Gₖₛ^{m}CₖₛAₖₛT_{ds}G_{ds}T_{ds}T_{ds}^{m}C_{ds}T_{dz}^{m}C_{dz}A_{ds}^{m}C_{dz}A_{dz}TₖₛTₖₛAₖ | 66 | 129 | 812 | 5 |
| 1467819 | Gₖₛ^{m}CₖₛAₖₛT_{ds}G_{dz}T_{dz}T_{ds}^{m}C_{ds}T_{ds}^{m}C_{dz}A_{dz}^{m}C_{ds}A_{ds}TₖₛTₖₛAₖ | 84 | n.d. | 181 | 5 |
| 1467820 | Gₖₛ^{m}CₖₛAₖₛT_{ds}G_{ds}T_{dz}T_{dz}^{m}C_{ds}T_{dz}^{m}C_{dz}A_{ds}^{m}C_{ds}A_{ds}TₖₛTₖₛAₖ | 78 | 231 | 335 | 5 |

A subscript “k” represents a cEt nucleoside, a subscript “d” represents a stereostandard DNA nucleoside, a subscript “s” indicates a phosphorothioate internucleoside linkage, a subscript “o” indicates a phosphodiester internucleoside linkage, and a subscript “z” represents an internucleoside linkage of formula IX, which is a mesyl phosphoramidate linkage. Subscripts of nucleotides having an internucleoside linkage of formula IX are bold and underlined. A superscript “m” before a C represents a 5-methyl Cytosine.

### Example 19: In Vitro Activity and Caspase Activation of Modified Oligonucleotides Comprising

### Modified Oligonucleotides

Modified oligonucleotides comprising multiple mesyl phosphoramidate internucleoside linkages (Formula IX), phosphorothioate internucleoside linkages, and phosphodiester internucleoside linkages, were synthesized and tested. The modified oligonucleotides are each 3-10-3 cEt gapmers with a sugar motif of: kkkddddddddddkkk (a 3-10-3 cEt motif) or kkkdyddddddddkkk, where “k” represents a cEt modified sugar moiety, “y” represents a 2′-OMe modified sugar moiety, and “d” represents a β-D-2′-deoxyribosyl sugar moiety. Each of the modified oligonucleotides has the same nucleobase sequence, GCATGTTCTCACATTA (SEQ ID NO: 5), which is 100% complementary to mouse CXCL12, GENBANK NT_039353.7 truncated from 69/430,515 to 69/445,350 (SEQ ID NO: 1), at position 6877 to 6892. Each internucleoside linkage is either a phosphorothioate internucleoside linkage (“s”) or a mesyl phosphoramidate internucleoside linkage (“z”).

### In Vitro Assays

In vitro activity of modified oligonucleotides described above was tested as described in Example 1. In vitro toxicity of modified oligonucleotides described above was determined as described in Example 3. The internucleoside linkages and sugar modifications of the modified nucleosides are indicated in subscripts in the table below.

TABLE 33
Design, Activity, and Tolerability of modified oligonucleotides having
multiple mesyl phosphoramidates linkages complementary to CXCL12

| | | CXCL12 | Caspase | SEQ |
|---|---|---|---|---|
| Compound | | IC₅₀ | Activation | ID |
| Number | Chemistry Notation (5′-3′) | (nM) | (% Mock) | NO. |
| 558807 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 54 | 2230 | 5 |
| 936053 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 144 | 102 | 5 |
| 1375418 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}T_{kz}T_{kz}Aₖ | 74 | 1983 | 5 |
| 1405468 | G_{kz}^{m}C_{kz}A_{kz}T_{ds}G_{ds}T_{ds}T_{ds}^{m}C_{ds}T_{ds}^{m}C_{ds}A_{ds}^{m}C_{ds}A_{ds}TₖₛTₖₛAₖ | 88 | 1480 | 5 |
| 1405469 | G_{kz}^{m}C_{kz}A_{kz}T_{ds}G_{ds}T_{ds}T_{ds}^{m}C_{ds}T_{ds}^{m}C_{ds}A_{ds}^{m}C_{ds}A_{ds}T_{kz}T_{kz}Aₖ | 58 | 1980 | 5 |
| 1405470 | G_{kz}^{m}C_{kz}A_{kz}T_{ds}G_{ys}T_{ds}T_{ds}^{m}C_{ds}T_{ds}^{m}C_{ds}A_{ds}^{m}C_{ds}A_{ds}TₖₛTₖₛAₖ | 86 | 133 | 5 |
| 1405471 | Gₖₛ^{m}CₖₛAₖₛT_{ds}G_{ys}T_{ds}T_{ds}^{m}C_{ds}T_{ds}^{m}C_{ds}A_{ds}^{m}C_{ds}A_{ds}T_{kz}T_{kz}Aₖ | 78 | 131 | 5 |
| 1405472 | G_{kz}^{m}C_{kz}A_{kz}T_{ds}G_{ys}T_{ds}T_{ds}^{m}C_{ds}T_{ds}^{m}C_{ds}A_{ds}^{m}C_{ds}A_{ds}T_{kz}T_{kz}Aₖ | 73 | 148 | 5 |
| 1405488 | Gₖₛ^{m}CₖₛAₖₛT_{ds}G_{ys}T_{dz}T_{dz}^{m}C_{ds}T_{ds}^{m}C_{ds}A_{ds}^{m}C_{ds}A_{ds}TₖₛTₖₛAₖ | 110 | 128 | 5 |
| 1405489 | Gₖₛ^{m}CₖₛAₖₛT_{ds}G_{yz}T_{dz}T_{ds}^{m}C_{ds}T_{ds}^{m}C_{ds}A_{ds}^{m}C_{ds}A_{ds}TₖₛTₖₛAₖ | 94 | 182 | 5 |
| 1405490 | Gₖₛ^{m}CₖₛAₖₛT_{dz}G_{yz}T_{ds}T_{ds}^{m}C_{ds}T_{ds}^{m}C_{ds}A_{ds}^{m}C_{ds}A_{ds}TₖₛTₖₛAₖ | 78 | 107 | 5 |
| 1405491 | Gₖₛ^{m}CₖₛAₖₛT_{ds}G_{ys}T_{ds}T_{ds}^{m}C_{ds}T_{ds}^{m}C_{dz}A_{dz}^{m}C_{ds}A_{ds}TₖₛTₖₛAₖ | 130 | 95 | 5 |
| 1405495 | Gₖₛ^{m}CₖₛAₖₛT_{ds}G_{ys}T_{dz}T_{ds}^{m}C_{ds}T_{ds}^{m}C_{ds}A_{ds}^{m}C_{ds}A_{ds}TₖₛTₖₛAₖ | 91 | 74 | 5 |
| 1405496 | Gₖₛ^{m}CₖₛAₖₛT_{ds}G_{yz}T_{ds}T_{ds}^{m}C_{ds}T_{ds}^{m}C_{ds}A_{ds}^{m}C_{ds}A_{ds}TₖₛTₖₛAₖ | 197 | 148 | 5 |
| 1405497 | Gₖₛ^{m}CₖₛAₖₛT_{ds}G_{ys}T_{ds}T_{ds}^{m}C_{ds}T_{ds}^{m}C_{ds}A_{dz}^{m}C_{ds}A_{ds}TₖₛTₖₛAₖ | 126 | 95 | 5 |

A subscript “k” represents a cEt nucleoside, a subscript “d” represents a stereostandard DNA nucleoside, a subscript “s” indicates a phosphorothioate internucleoside linkage, a subscript “z” represents an internucleoside linkage of formula IX, which is a mesyl phosphoramidate linkage, and a subscript “y” represents a 2′-OMe modified nucleoside. Subscripts of nucleotides having an internucleoside linkage of formula IX are bold and underlined. A superscript “m” before a C represents a 5-methyl Cytosine.

### Example 20: Design and Activity of siRNA to HRPT1 Having Mesyl Phosphoramidate Internucleoside Linkages In Vitro

siRNA

Double-stranded siRNA comprising modified oligonucleotides having mesyl phosphoramidate internucleoside linkages (Formula IX) in the sense and/or antisense strands were synthesized and tested. Each internucleoside linkage is either a phosphorothioate internucleoside linkage (“s”), a phosphodiester internucleoside linkage (“o”), or a mesyl phosphoramidate internucleoside linkage (“z”).

Each antisense strand has the sequence AUAAAAUCUACAGUCAUAGGAAU (SEQ ID NO: 21) and is 100% complementary to GenBlank NM_000194.2 (SEQ ID NO: 22) from 444 to 466, and each antisense strand has a 5′-phosphate. Each sense strand has the sequence UCCUAUGACUGUAGAUUUUAU (SEQ ID NO: 23) and is 100% identical to GenBlank NM_000194.2 (SEQID NO: 22) from 446 to466. Compound No. 1151789, 1337113, 1471455, and 1515982 comprise a 3′-linked C7 amino modifier (Glen Research), shown below:

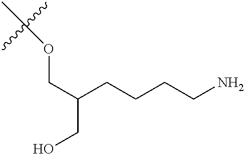 [SMILES: *OCC(CO)CCCCN]

Compound No. 1448688 further comprises a GalNAc conjugated at the 3′-oxygen of the oligonucleotide via a THA linker as shown below:

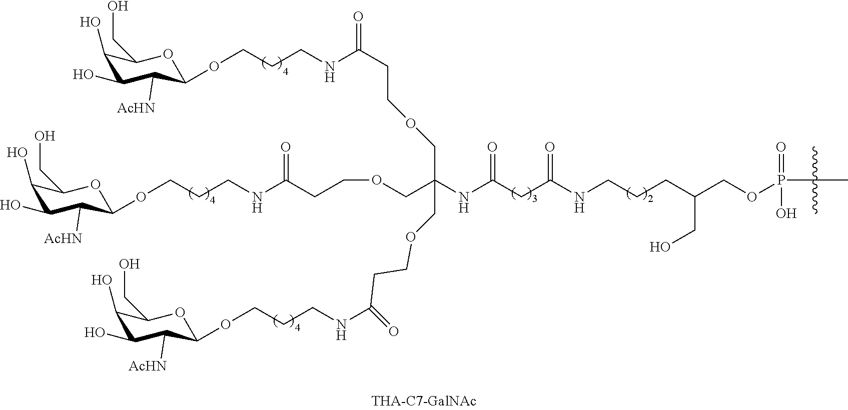

TABLE 34
Design of antisense strand modified oligonucleotides targeted to
human/mouse HRPT1 having mesyl phosphoramidate linkages

| Compound | | SEQ ID |
|---|---|---|
| ID | Chemical Notation (5′ to 3′) | NO: |
| 1073762 | p.A_{yo}U_{fo}A_{yo}A_{fo}A_{yo}A_{fo}U_{yo}C_{fo}U_{yo}A_{fo}C_{yo}A_{fo}G_{yo}U_{fo}C_{yo}A_{fo}U_{yo}A_{fo}G_{yo}G_{fo}A_{ys}A_{fs}U_{y} | 21 |
| 1337111 | p.A_{ys}U_{fs}A_{yo}A_{fo}A_{yo}A_{fo}U_{yo}C_{fo}U_{yo}A_{fo}C_{yo}A_{fo}G_{yo}U_{fo}C_{yo}A_{fo}U_{yo}A_{fo}G_{yo}G_{fo}A_{ys}A_{fs}U_{y} | 21 |
| 1465680 | p.A_{yz}U_{fs}A_{yo}A_{fo}A_{yo}A_{fo}U_{yo}C_{fo}U_{yo}A_{fo}C_{yo}A_{fo}G_{yo}U_{fo}C_{yo}A_{fo}U_{yo}A_{fo}G_{yo}G_{fo}A_{ys}A_{fs}U_{y} | 21 |
| 1465681 | p.A_{ys}U_{fz}A_{yo}A_{fo}A_{yo}A_{fo}U_{yo}C_{fo}U_{yo}A_{fo}C_{yo}A_{fo}G_{yo}U_{fo}C_{yo}A_{fo}U_{yo}A_{fo}G_{yo}G_{fo}A_{ys}A_{fs}U_{y} | 21 |
| 1465682 | p.A_{yo}U_{fo}A_{yo}A_{fo}A_{yo}A_{fo}U_{yo}C_{fo}U_{yo}A_{fo}C_{yo}A_{fo}G_{yo}U_{fo}C_{yo}A_{fo}U_{yo}A_{fo}G_{yo}G_{fz}A_{yz}A_{fz}U_{y} | 21 |
| 1465683 | p.A_{yo}U_{fo}A_{yo}A_{fo}A_{yo}A_{fo}U_{yo}C_{fo}U_{yo}A_{fo}C_{yo}A_{fo}G_{yo}U_{fo}C_{yo}A_{fo}U_{yz}A_{fz}G_{yz}G_{fo}A_{ys}A_{fs}U_{y} | 21 |
| 1465684 | p.A_{yo}U_{fo}A_{yo}A_{fo}A_{yo}A_{fo}U_{yo}C_{fo}U_{yo}A_{fo}C_{yo}A_{fo}G_{yo}U_{fz}C_{yz}A_{fz}U_{yo}A_{fo}G_{yo}G_{fo}A_{ys}A_{fs}U_{y} | 21 |
| 1465685 | p.A_{yo}U_{fo}A_{yo}A_{fo}A_{yo}A_{fo}U_{yo}C_{fo}U_{yo}A_{fo}C_{yz}A_{fz}G_{yz}U_{fo}C_{yo}A_{fo}U_{yo}A_{fo}G_{yo}G_{fo}A_{ys}A_{fs}U_{y} | 21 |
| 1465686 | p.A_{yo}U_{fo}A_{yo}A_{fo}A_{yo}A_{fo}U_{yo}C_{fz}U_{yz}A_{fz}C_{yo}A_{fo}G_{yo}U_{fo}C_{yo}A_{fo}U_{yo}A_{fo}G_{yo}G_{fo}A_{ys}A_{fs}U_{y} | 21 |
| 1465687 | p.A_{yo}U_{fo}A_{yo}A_{fo}A_{yz}A_{fz}U_{yz}C_{fo}U_{yo}A_{fo}C_{yo}A_{fo}G_{yo}U_{fo}C_{yo}A_{fo}U_{yo}A_{fo}G_{yo}G_{fo}A_{ys}A_{fs}U_{y} | 21 |
| 1449196 | p.A_{ys}U_{fs}A_{yo}A_{yo}A_{yo}A_{fo}U_{yo}C_{yo}U_{yo}A_{yo}C_{yo}A_{yo}G_{yo}U_{fo}C_{yo}A_{fo}U_{yo}A_{yo}G_{yo}G_{yo}A_{ys}A_{ys}U_{y} | 21 |
| 1466140 | p.A_{ys}U_{fs}A_{yo}A_{yo}A_{yo}A_{fo}U_{yo}C_{yo}U_{yo}A_{yo}C_{yo}A_{yo}G_{yo}U_{fo}C_{yo}A_{fo}U_{yo}A_{yo}G_{yo}G_{yo}A_{yz}A_{yz}U_{y} | 21 |
| 1515975 | p.A_{ys}U_{fs}A_{yo}A_{yo}A_{yo}A_{fz}U_{yo}C_{yo}U_{yo}A_{yo}C_{yo}A_{yo}G_{yo}U_{fz}C_{yo}A_{fz}U_{yo}A_{yo}G_{yo}G_{yo}A_{ys}A_{ys}U_{y} | 21 |
| 1515976 | p.A_{ys}U_{fz}A_{yo}A_{yo}A_{yo}A_{fz}U_{yo}C_{yo}U_{yo}A_{yo}C_{yo}A_{yo}G_{yo}u_{fz}C_{yo}A_{fz}U_{yo}A_{yo}G_{yo}G_{yo}A_{ys}A_{ys}U_{y} | 21 |

A “p.” represents a 5′-phosphate. A subscript “f” represents a 2′-F modified nucleoside, a subscript “y” represents a 2′-OMe modified nucleoside, a subscript “s” indicates a phosphorothioate internucleoside linkage, a subscript “o” represents a phosphodiester internucleoside linkage, a subscript “z” represents an internucleoside linkage of formula IX, which is a mesyl phosphoramidate linkage. Subscripts of nucleotides having a phosphoramidate internucleoside linkage of generic Formula XVII are bold and underlined.

TABLE 35
Design of sense strand modified oligonucleotides targeted to
human/mouse HRPT1 having mesyl phosphoramidate linkages

| Compound | | SEQ ID |
|---|---|---|
| ID | Chemical Notation (5′ to 3′) | NO: |
| 1151789 | U_{fo}C_{yo}C_{fo}U_{yo}A_{fo}U_{yo}G_{fo}A_{yo}C_{fo}U_{yo}G_{fo}U_{yo}A_{fo}G_{yo}A_{fo}U_{yo}U_{fo}U_{yo}U_{fo}A_{yo}U_{fo} | 23 |
| | [3′-amino C7 Tag] | |
| 1448688 | U_{ys}C_{ys}C_{yo}U_{yo}A_{yo}U_{yo}G_{fo}A_{yo}C_{fo}U_{fo}G_{fo}U_{yo}A_{yo}G_{yo}A_{yo}U_{yo}U_{yo}U_{yo}U_{yo}A_{yo}U_{y} | 23 |
| | THA-C7-GalNAc | |
| 1471455 | U_{yz}C_{yz}C_{yo}U_{yo}A_{yo}U_{yo}G_{fo}A_{yo}C_{fo}U_{fo}G_{fo}U_{yo}A_{yo}G_{yo}A_{yo}U_{yo}U_{yo}U_{yo}U_{yz}A_{yz}U_{yo} | 23 |
| | [3′-amino C7 Tag] | |
| 1515982 | U_{ys}C_{ys}C_{yo}U_{yo}A_{yo}U_{yo}G_{fz}A_{yo}C_{fz}U_{fz}G_{fz}U_{yo}A_{yo}G_{yo}A_{yo}U_{yo}U_{yo}U_{yo}U_{yo}A_{yo}U_{y} | 23 |
| | [3′-amino C7 tag] | |

A subscript “f” represents a 2′-F modified nucleoside, a subscript “y” represents a 2′-OMe modified nucleoside, a subscript “s” indicates a phosphorothioate internucleoside linkage, a subscript “o” represents a phosphodiester internucleoside linkage.

### Activity Assay

Activity of various siRNA formed by annealing one antisense strand and the sense strand 1151789 described above was tested in HeLa cells. HeLa cells were transfected with 6 μL/mL of siRNA using RNAiMAX for 5 hours. RNA was isolated and RNA expression was analyzed via RT-qPCR using primer probe set Hs02800695_ml(ThennoFisher).

TABLE 36
Activity of siRNAs having mesyl phosphoramidate
linkages against human HPRT1

| Antisense Strand | Sense Strand | IC50 (nM) |
|---|---|---|
| 1073762 | 1151789 | 0.009 |
| 1337111 | 1151789 | 0.037 |
| 1465680 | 1151789 | 0.083 |
| 1465681 | 1151789 | 0.051 |
| 1465682 | 1151789 | 0.016 |
| 1465683 | 1151789 | 0.023 |
| 1465684 | 1151789 | 0.042 |
| 1465685 | 1151789 | 0.045 |
| 1465686 | 1151789 | 0.082 |
| 1465687 | 1151789 | 0.382 |

TABLE 37
Activity of siRNAs having mesyl phosphoramidate
linkages against HPRT1

| Antisense Strand | Sense Strand | IC₅₀ (nM) |
|---|---|---|
| 1073762 | 1337113 | 0.075 |
| 1449196 | 1471455 | 0.040 |
| 1466140 | 1471455 | 0.049 |
| 1515975 | 1448688 | 0.066 |
| 1515976 | 1448688 | 0.137 |
| 1515975 | 1515982 | 2.531 |
| 1515976 | 1515982 | 3.875 |
| 1449196 | 1515982 | 0.190 |

### Example 21: Measurement of Viscosity of Modified Oligonucleotides

The viscosity of modified oligonucleotides comprising mesyl phosphoramidate internucleoside linkages was compared to the viscosity of modified oligonucleotides having only phosphorothioate internucleoside linkages. Each nucleobase in the table below is represented by N, representing A, G, T, or ^{m}C. Each of oligonucleotides A1, A2, and A3 have the same sequence, and each of oligonucleotides B1, B2, and B3 have the same sequence. Oligonucleotides (32-38 mg) were weighed into a glass vial; approximately 100 μL of water was added, and the modified oligonucleotide was dissolved into solution by heating the vial to 55° C. Part (75 μL) of the pre-heated sample was pipetted to a micro-viscometer (PAC Cambridge Viscosity Viscometer). The temperature of the micro-viscometer was set to 25° C. and the viscosity of the sample was measured. The entire 75 μL of sample was them combined with the remaining portion of the sample was diluted appropriately for UV reading at 260 nM (Cary UV instrument). The data below indicates that the incorporation of mesyl phosphoramidate linkages in the gap can reduce viscosity.

TABLE 38
Viscosity

| Compound | | Viscosity | SEQ ID |
|---|---|---|---|
| ID | Chemistry Notation | (cP) | NO: |
| A1 | NₖₛNₖₛNₖₛN_{ds}N_{ds}N_{ds}N_{ds}N_{ds}N_{ds}N_{ds}N_{ds}N_{ds}N_{ds}NₖₛNₖₛNₖ | 90.6 | 29 |
| A2 | NₖₛNₖₛNₖₛN_{dz}N_{dz}N_{dz}N_{dz}N_{ds}N_{ds}N_{ds}N_{ds}N_{ds}N_{ds}NₖₛNₖₛNₖ | 46.0 | 29 |
| A3 | N_{kz}N_{kz}NₖₛN_{ds}N_{ds}N_{ds}N_{ds}N_{ds}N_{ds}N_{ds}N_{ds}N_{ds}N_{dz}N_{kz}N_{kz}Nₖ | 96.2 | 29 |
| B1 | NₖₛNₖₛNₖₛN_{ds}N_{ds}N_{ds}N_{ds}N_{ds}N_{ds}N_{ds}N_{ds}N_{ds}NₖₛNₖₛNₖₛNₑ | 44.5 | 29 |
| B2 | NₖₛNₖₛNₖₛN_{dz}N_{dz}N_{dz}N_{dz}N_{ds}N_{ds}N_{ds}N_{ds}N_{ds}NₖₛNₖₛNₖₛNₑ | 17.0 | 29 |
| B3 | N_{kz}N_{kz}NₖₛN_{ds}N_{ds}N_{ds}N_{ds}N_{ds}N_{ds}N_{ds}N_{ds}N_{ds}N_{kz}N_{kz}N_{kz}Nₑ | 9.6 | 29 |

A subscript “k” represents a cEt modified nucleoside, a subscript “d” represents a stereostandard DNA nucleoside, a subscript “e” represents a 2′-MOE modified nucleoside, a subscript “s” indicates a phosphorothioate internucleoside and a subscript “z” represents an internucleoside linkage of formula IX

### Example 22: Synthesis and In Vivo Activity of siRNA to SOD-1 Having a C16 Modified-Phosphoramidate Internucleoside Linkage

Double-stranded siRNA comprising a modified oligonucleotide having a mesyl phosphoramidate internucleoside linkage of Formula XIX in the sense strand was synthesized and tested in vivo.

For the sense strand, each internucleoside linkage is either a phosphorothioate internucleoside linkage (“s”), a phosphodiester internucleoside linkage (“o”), or a modified phosphoramidate internucleoside linkage having a C16 moiety, as shown below (“XIX”).

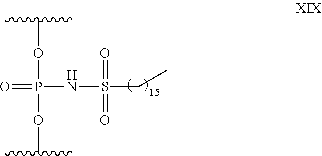

### Synthesis

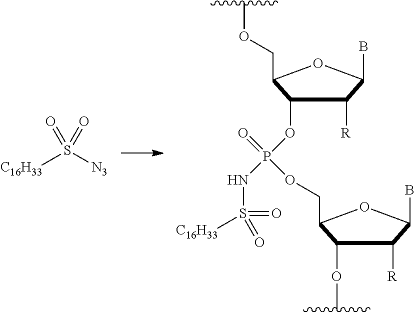

Oligonucleotides were synthesized on a 40 μmol scale using Nittophase UnyLinker support (405 μmol/g) on an AKTA 10 Oligopilot. Fully protected nucleoside phosphoramidites were incorporated using standard solid-phase oligonucleotide synthesis, i.e. 15% dichloroacetic acid in toluene for deblocking, 1 M 4,5-dicyanoimidazole 0.1 M N-methylimidazole in acetonitrile as activator for phosphoramidite couplings, 20% acetic anhydride in THF and 10% 1-methylimidazole in THF/pyridine for capping and 20% tBuOOH in acetonitrile for oxidation or 0.1 M xanthane hydride in pyridine:acetonitrile 3:2 (v:v) for thiolation. Oxidation to form the hexadecyl sulfonyl phosphoramidate linkage (Formula XIX) was performed using 0.5 M C₁₆H₃₃SO₂N₃(hexadecyl sulfonyl azide) in acetonitrile:toluene 1:1 (v/v) with a 90 minute recycle time. Phosphoramidites were dissolved to 0.1 M in acetonitrile:toluene 1:1 (v:v) and incorporated using a 10 min coupling recycling time. At the end of the solid phase synthesis cyanoethyl protecting groups were removed by a 30 min treatment with 20% diethylamine in toluene. Oligonucleotides were deprotected and cleaved using conc. aq. ammonia at room temperature for 48 h.

siRNA Design

Double-stranded siRNA compounds were formed by annealing one antisense strand and one sense strand described below. Compound No. 1521629 is the antisense strand, wherein the sequence (from 5′ to 3′) UUAGAGUGAGGAUUAAAAUGAG (SEQ ID NO: 33) is 100% complementary to the genomic sequence of rat SOD-1, SEQ ID NO: 34, the complement of GENBANK Accession No. NW_047354.2, truncated from 29807000 to 29819000, at position 6230 to 6251. The non-complementary overhang is highlighted in bold in the table below.

TABLE 39
Design of antisense strand of modified oligonucleotides

| Compound | | SEQ ID |
|---|---|---|
| ID | Chemistry Notation (5′ to 3′) | NO. |
| 1521629 | vP-TₑₛU_{fs}U_{yo}A_{yo}G_{yo}A_{fo}G_{yo}U_{fo}G_{fo}A_{yo}G_{yo}G_{yo}A_{yo}U_{fo}U_{yo}A_{fo}A_{yo}A_{yo}A_{yo}U_{yo}G_{ys}A_{ys}G_{y} | 33 |

In the table above, a subscript “f” represents a 2′-F modified nucleoside, a subscript “y” represents a 2′-OMe modified nucleoside, a subscript “e” represents a 2′-MOE modified nucleoside, a subscript “s” indicates a phosphorothioate internucleoside linkage, and a subscript “o” represents a phosphodiester internucleoside linkage. Compound No. 1521629 contains a vinyl phosphonate (vP) moiety on the 5′-end.

TABLE 40
Design of sense strand of modified oligonucleotides

| Compound | | SEQ ID |
|---|---|---|
| ID | Chemistry Notation (5′ to 3′) | NO. |
| 1523488 | C_{ys}A_{ys}U_{yo}U_{yo}U_{yo}U_{yo}A_{fo}A_{yo}U_{fo}C_{fo}C_{fo}U_{yo}C_{yo}A_{yo}C_{yo}U_{yo}C_{yo}U_{yo}A_{ys}A_{ys}A_{y} | 30 |
| 1524752 | C_{ys}A_{ys}U_{yo}U_{yo}U_{yo}U_{y[XIX]}A_{fo}A_{yo}U_{fo}C_{fo}C_{fo}U_{yo}C_{yo}A_{yo}C_{yo}U_{yo}C_{yo}U_{yo}A_{ys}A_{ys}A_{y} | 30 |

In the table above, a subscript “f” represents a 2′-F modified nucleoside, a subscript “y” represents a 2′-OMe modified nucleoside, a subscript “s” indicates a phosphorothioate internucleoside linkage, and a subscript “o” represents a phosphodiester internucleoside linkage. A subscript “[XIX]” represents an internucleoside linkage of Formula XIX. Subscripts of nucleotides having a substituted phosphoramidate internucleoside linkage of generic Formula XVII are bold and underlined.

### In Vivo

For the in vivo activity study in the table below, 2-4 Sprague Dawley rats per group were administered siRNA by intrathecal injection at a total dose of 7.5, 30, 75, 90, 300, or 900 μg. One group of four Sprague Dawley rats was injected with PBS as a control.

RNA analysis

Two weeks post treatment, rats were sacrificed and RNA was extracted from cortical brain tissue and spinal cord for real-time qPCR analysis of SOD-1 RNA. Primer probe set RTS592 (forward sequence CGGATGAAGAGAGGCATGTTG, designated herein as SEQ ID NO: 35; reverse sequence TTGGCCACACCGTCCTTT, designated herein as SEQ ID NO: 36; probe sequence AGACCTGGGCAATGTGGCTGCTG, designated herein as SEQ ID NO: 37) was used to determine the amount of SOD-1 RNA. The median effective dose (ED₅₀) of each siRNA was calculated in GraphPad Prism using the equation “log(agonist) vs. response—Find ECanything Least squares fit.”

As shown in the table below, treatment with siRNA with a C16 modified-phosphoramidate internucleoside linkage resulted in increased potency in both the cortex and the spinal cord compared to an siRNA lacking a C16 modification.

TABLE 41
In vivo activity of siRNA to SOD-1

| Antisense | Sense | Cortex | Spinal Cord |
|---|---|---|---|
| Strand | Strand | ED₅₀ (μg) | ED₅₀ (μg) |
| 1521629 | 1523488 | 998 | 48 |
| 1521629 | 1524752 | 223 | 15 |

### Example 23: Design, Activity, and Tolerability of Modified Oligonucleotides with Mesyl Phosphoramidate Internucleoside Linkages Complementary to Factor XI In Vivo

### Design of Modified Oligonucleotides

GalNAc-conjugated modified oligonucleotides comprising multiple mesyl phosphoramidate internucleoside linkages (Formula IX) and phosphorothioate internucleoside linkages were synthesized and tested. The modified oligonucleotides are 100% complementary to the complement of mouse Factor XI, GENBANK Accession No. NT_039460.6 truncated from 6086000 to 6111000 (SEQ ID NO: 38), at position 22323 to 22338. Each internucleoside linkage is either a phosphorothioate internucleoside linkage (“s”) or a mesyl phosphoramidate internucleoside linkage (“z”).

The modified oligonucleotides in the table below contain the GalNAc moiety conjugated to the 3-oxygen as shown below:

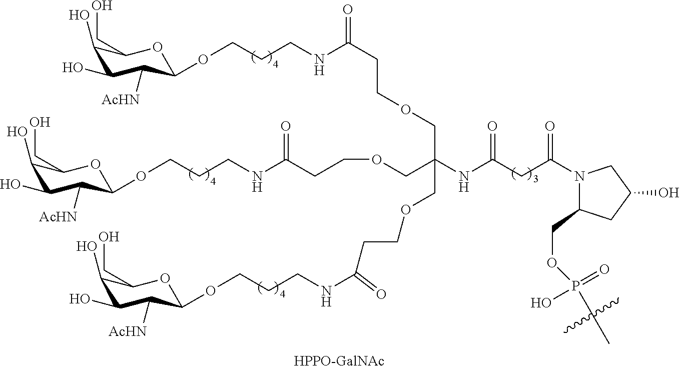

TABLE 42
Design of modified oligonucleotides with mesyl
phosphoramidate internucleoside linkages

| Compound | | SEQ ID |
|---|---|---|
| ID | Chemistry Notation (5′ to 3′) | NO. |
| 1468445 | ^{m}CₖₛTₖₛGₖₛT_{ds}T_{ds}T_{ds}G_{ds}A_{ds}G_{ds}T_{ds}T_{ds}T_{ds}T_{ds}^{m}CₖₛTₖₛ^{m}Cₖ- | 39 |
| | HPPO-GalNAc | |
| 1506051 | ^{m}CₖₛTₖₛGₖₛT_{ds}U_{ys}T_{ds}G_{ds}A_{ds}G_{ds}T_{ds}T_{ds}T_{ds}T_{ds}^{m}CₖₛTₖₛ^{m}Cₖ- | 40 |
| | HPPO-GalNAc | |
| 1505717 | ^{m}CₖₛTₖₛGₖₛT_{dz}T_{dz}T_{dz}G_{dz}A_{ds}G_{ds}T_{ds}T_{ds}T_{ds}T_{ds}^{m}CₖₛTₖₛ^{m}Cₖ- | 39 |
| | HPPO-GalNAc | |

A subscript “k” represents a cEt nucleoside, a subscript “d” represents a stereo-standard DNA nucleoside, a subscript “s” indicates a phosphorothioate internucleoside linkage, and a subscript “z” represents an internucleoside linkage of formula IX, which is a mesyl phosphoramidate linkage. Subscripts of nucleotides having an internucleoside linkage of formula IX are bold and underlined. A superscript “m” before a C represents a 5-methylcytosine. HPPO-GalNAc represents a 3′-GalNAc moiety.

### Study Design

For the in vivo activity and tolerability study in the table below, 3 BALB/C mice per group were administered a single dose of modified oligonucleotide by subcutaneous injection and sacrificed after 72 hours. Modified oligonucleotides were administered at 0.31, 0.93, 2.78, 8.33, or 25 mg/kg. One group of four BALB/C mice was injected with PBS.

Liver tissue was collected, mRNA was isolated, and levels of FXI in liver samples were measured by quantitative RTPCR with mouse primer probe set RTS2898 (forward sequence: ACATGACAGGCGCGATCTCT, SEQ ID NO: 41; reverse sequence: TCTAGGTTCACGTACACATCTTTGC, SEQ ID NO: 42; probe sequence: TTCCTTCAAGCAATGCCCTCAGCAAT, SEQ ID NO: 43). Expression levels were normalized to total RNA content as measured with RIBOGREEN®. ED₅₀ values were calculated by a least squares fit of data in GraphPad Prism using the equation “[Inhibitor]vs. response—Variable slope (four parameters)” and are presented in the table below. Plasma ALT was also measured and is presented in the table below. Elevations in ALT are associated with liver toxicity. The PBS treated mice have an ALT of 36.75 IU/L.

TABLE 43
In vivo activity and toxicity of modified
oligonucleotides complementary to FXI

| Compound | in vivo FXI ED₅₀ | ALT @ 25 |
|---|---|---|
| ID | liver (mg/kg) | mg/kg (IU/L) |
| 1468445 | 1.1 | 42 |
| 1506051 | 31 | 63 |
| 1505717 | 1.4 | 52 |

### Example 24: Design, Activity, and Tolerability of Modified Oligonucleotides with Mesyl Phosphoramidate Internucleoside Linkages Complementary to HDAC2 In Vivo

### Design of Modified Oligonucleotides

GalNAc-conjugated modified oligonucleotides having multiple mesyl phosphoramidate internucleoside linkages (Formula IX) and phosphorothioate internucleoside linkages were synthesized and tested. The modified oligonucleotides are 100% complementary to mouse HDAC2 GENBANK Accession No. NT_039492.7 truncated from 29396000 to 29430000 (SEQ ID NO: 44), at position 19150 to 19165. Each internucleoside linkage is either a phosphorothioate internucleoside linkage (“s”) or a mesyl phosphoramidate internucleoside linkage (“z”).

The modified oligonucleotides in the table below contain the GalNAc moiety conjugated to the 3-oxygen as shown below:

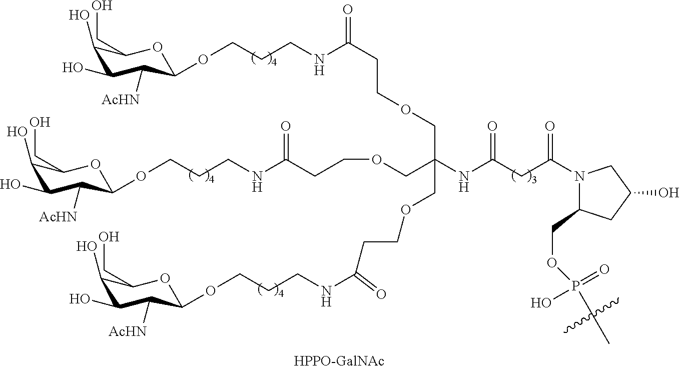

TABLE 44
Design of modified oligonucleotides with
mesyl phosphoramidate internucleoside linkages

| Com- | | SEQ |
|---|---|---|
| pound | | ID |
| ID | Chemistry Notation (5′ to 3′) | NO. |
| 1506050 | Aₖₛ^{m}Cₖₛ^{m}Cₖₛ^{m}C_{ds}U_{ys}^{m}C_{ds}A_{ds}A_{ds}G_{ds}T_{ds}^{m}C_{ds}T_{ds}^{m}C_{ds}^{m} | 45 |
| | CₖₛTₖₛGₖ HPPO-GalNAc | |
| 1505715 | Aₖₛ^{m}Cₖₛ^{m}Cₖₛ^{m}C_{dz}T_{dz}^{m}C_{dz}A_{dz}A_{ds}G_{ds}T_{ds}^{m}C_{ds}T_{ds}^{m} | 46 |
| | C_{ds}^{m}CₖₛTₖₛGₖ HPPO-GalNAc | |

A subscript “k” represents a cEt nucleoside, a subscript “d” represents a stereo-standard DNA nucleoside, a subscript “y” represents a 2′-OMe modified nucleoside, a subscript “s” indicates a phosphorothioate internucleoside linkage, and a subscript “z” represents an internucleoside linkage of formula IX, which is a mesyl phosphoramidate linkage. Subscripts of nucleotides having an internucleoside linkage of formula IX are bold and underlined. A superscript “m” before a C represents a 5-methylcytosine.

### Study Design

For the in vivo activity and tolerability study in the table below, 3 BALB/C mice per group were administered a single dose of modified oligonucleotide by subcutaneous injection and sacrificed after 72 hours. Modified oligonucleotides were dosed at 0.3, 0.9, 2.8, 8.3, or 25 mg/kg. One group of four BALB/C mice was injected with PBS.

Liver tissue was collected, mRNA was isolated, and levels of HDAC2 in liver samples were measured by quantitative RTPCR with mouse primer probe set Mm00515108_ml (Applied Biosystems). Expression levels were normalized to total RNA as measured with RIBOGREEN®. ED₅₀ values were calculated by a least squares fit of data in GraphPad Prism using the equation “[Inhibitor]vs. response—Variable slope (four parameters)” and are presented in the table below. Plasma ALT was also measured and is presented in the table below. Elevations in ALT are associated with liver toxicity. The PBS treated mice have an ALT of 53 IU/L.

TABLE 45
In vivo activity and toxicity of modified
oligonucleotides complementary to HDAC2

| Compound | in vivo HDAC2 | ALT @ 25 |
|---|---|---|
| ID | ED₅₀ liver (mg/kg) | mg/kg (IU/L) |
| 1506050 | 7.5 | 62 |
| 1505715 | 3.9 | 81 |

### Example 25: Design, activity, and tolerability of modified oligonucleotides with mesyl phosphoramidate internucleoside linkages complementary to DNM2 in vivo

### Design of Modified Oligonucleotides

GalNAc-conjugated modified oligonucleotides having multiple mesyl phosphoramidate internucleoside linkages (Formula IX) and phosphorothioate internucleoside linkages were synthesized and tested. Each of the modified oligonucleotides is 100% complementary to mouse DNM2, GENBANK NC_000075.6 truncated from 21422001 to 21511000 (SEQ ID NO: 47), at position 3046 to 3061. Each internucleoside linkage is either a phosphorothioate internucleoside linkage (“s”) or a mesyl phosphoramidate internucleoside linkage (“z”).

The modified oligonucleotides in the table below contain the GalNAc moiety conjugated to the 3-oxygen as shown below:

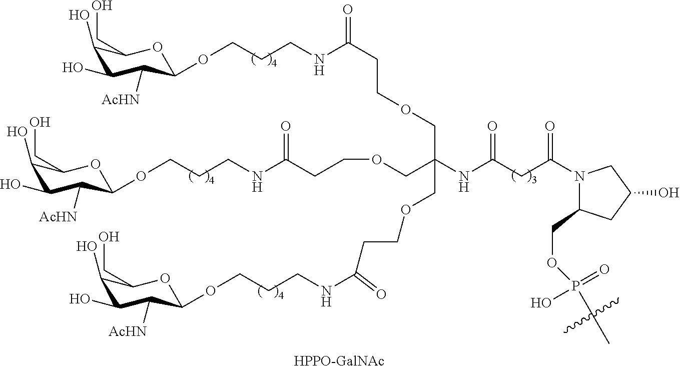

TABLE 46
Design of modified oligonucleotides with
mesyl phosphoramidate internucleoside linkages

| | | SEQ |
|---|---|---|
| Compound | | ID |
| ID | Chemistry Notation (5′ to 3′) | NO. |
| 1506053 | AₖₛGₖₛAₖₛ^{m}C_{ds}U_{ys}^{m}C_{ds}T_{ds}^{m}C_{ds}G_{ds}G_{ds}T_{ds}T_{ds}^{m}C_{ds}^{m} | 48 |
| | CₖₛGₖₛAₖ-HPPO-GalNAc | |
| 1505722 | AₖₛGₖₛAₖₛ^{m}C_{dz}T_{dz}^{m}C_{dz}T_{dz}^{m}C_{ds}G_{ds}G_{ds}T_{ds}T_{ds}^{m} | 49 |
| | C_{ds}^{m}CₖₛGₖₛAₖ-HPPO-GalNAc | |

A subscript “k” represents a cEt nucleoside, a subscript “d” represents a stereo-standard DNA nucleoside, a subscript “s” indicates a phosphorothioate internucleoside linkage, and a subscript “z” represents an internucleoside linkage of formula IX, which is a mesyl phosphoramidate linkage. Subscripts of nucleotides having an internucleoside linkage of formula IX are bold and underlined. A superscript “m” before a C represents a 5-methylcytosine.

### Study Design

For the in vivo activity and tolerability study in the table below, 3 BALB/C mice per group were administered a single dose of modified oligonucleotide by subcutaneous injection and sacrificed after 72 hours. Modified oligonucleotides were dosed at 0.1, 0.3, 0.9, 2.8, 8.3, or 25 mg/kg. One group of four BALB/C mice was injected with PBS.

Liver tissue was collected, mRNA was isolated, and levels of DNM2 in liver samples were measured by quantitative RTPCR with primer probe set RTS36436 (forward sequence: AGAGGAGACCGAGCGAAT, SEQ ID NO: 50; reverse sequence: CATGGTTTGTGTTGATGTACGAC, SEQ ID NO: 51; probe sequence: CCTACATCAGGGAGCGAGAAGGGA, SEQ ID NO: 52). Expression levels were normalized to total RNA as measured with RIBOGREEN®. ED₅₀ values were calculated by a least squares fit of data in GraphPad Prism using the equation “[Inhibitor]vs. response—Variable slope (four parameters)” and are presented in the table below. Plasma ALT was also measured and is presented in the table below. Elevations in ALT are associated with liver toxicity. The PBS treated mice have an ALT of 38.75 IU/L.

TABLE 47
In vivo activity and toxicity of modified
oligonucleotides complementary to DNM2

| Compound | in vivo DNM2 ED₅₀ | ALT @ 25 |
|---|---|---|
| ID | liver (mg/kg) | mg/kg (IU/L) |
| 1506053 | 0.94 | 35 |
| 1505722 | 0.53 | 110 |

### Example 26: Design and Activity of Modified Oligonucleotides Complementary to Mouse FXII In Vitro and a Single Dose Duration of Action Study

### Design of Modified Oligonucleotides

GalNAc-conjugated modified oligonucleotides comprising multiple mesyl phosphoramidate internucleoside linkages (Formula IX) and phosphorothioate internucleoside linkages were synthesized and tested. Each of the modified oligonucleotides has the same nucleobase sequence, AGCACTTTATTGAGTT (SEQ ID NO: 53), which is 100% complementary to mouse FXII, the complement of GENBANK NC_000079.6 truncated from 55415001 to 55430000 (SEQ ID NO: 54), at position 12009 to 12024. Each internucleoside linkage is either a phosphorothioate internucleoside linkage (“s”), a phosphodiester internucleoside linkage (“o”), or a mesyl phosphoramidate internucleoside linkage (“z”).

The GalNAc moiety is conjugated to the 5′ oxygen of compound 1447171 via a THA linker, as shown below:

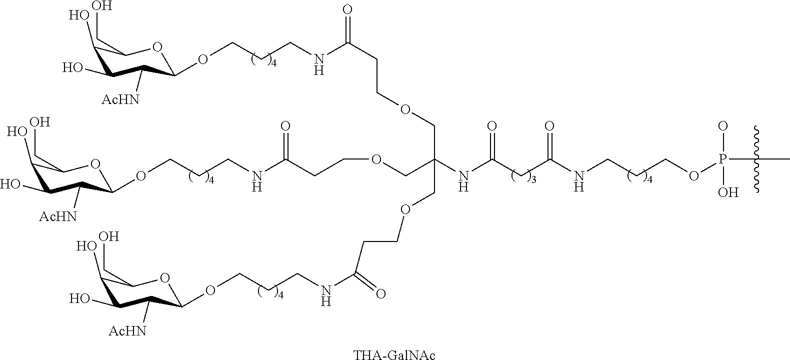

Aside from compound 1447171, the modified oligonucleotides in the table below contain the GalNAc moiety conjugated to the 3′-oxygen as shown below:

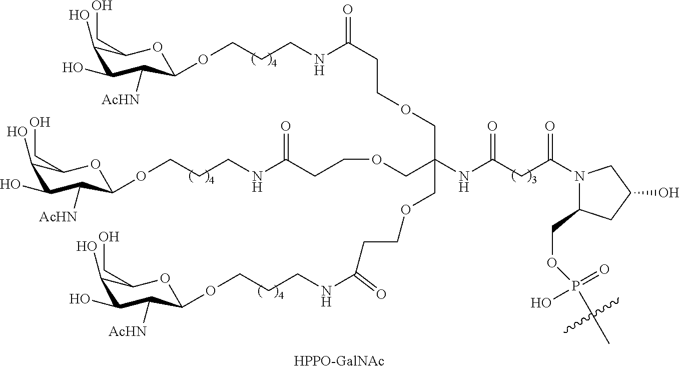

### Activity Assay

Activity of antisense oligonucleotides was tested in primary mouse hepatocytes. Primary mouse hepatocytes cells were transfected with lipofectamine. Each modified oligonucleotide was transfected at a starting concentration of 200 nM with 5-fold serial dilutions for a total of 8 dilutions. After a treatment period of approximately 24 hours, RNA was isolated and RNA expression was analyzed via quantitative RTPCR using primer probe set RTS2959 (forward sequence CAAAGGAGGGACATGTATCAACAC, SEQ ID NO: 91; reverse sequence: CTGGCAATGTTTCCCAGTGA, SEQ ID NO: 92; probe sequence: CCCAATGGGCCACACTGTCTCTGC, SEQ ID NO: 93). FXII RNA levels were normalized to total GAPDH. Activity expressed as half maximal inhibitory concentration (IC₅₀) was calculated using the log (inhibitor) vs normalized response—Variable slope function in GraphPad Prism 7.

TABLE 48
Design and in vitro activity of modified
oligonucleotides complementary to mouse FXII

| Compound | | IC₅₀ | SEQ ID |
|---|---|---|---|
| ID | Chemistry Notation (5′ to 3′) | (nM) | NO. |
| 1447171 | THA-GalNAc- | n.d. | 53 |
| | AₖₛGₖₛ^{m}CₖₛA_{ds}^{m}C_{ds}T_{ds}T_{ds}T_{ds}A_{ds}T_{ds}T_{ds}G_{ds}A_{ds}GₖₛTₖₛTₖ | | |
| 1525915 | AₖₛGₖₛ^{m}CₖₛA_{ds}^{m}C_{ds}T_{ds}T_{ds}T_{ds}A_{ds}T_{ds}T_{ds}G_{ds}A_{ds}GₖₛTₖₛTₖ- | 0.0231 | 53 |
| | HPPO-GalNAc | | |
| 1525921 | AₖₛGₖₒ^{m}CₖₒA_{ds}^{m}C_{ds}T_{ds}T_{ds}T_{ds}A_{ds}T_{ds}T_{ds}G_{ds}A_{ds}GₖₒTₖₛTₖ- | 0.0175 | 53 |
| | HPPO-GalNAc | | |
| 1525920 | AₖₒGₖₒ^{m}CₖₒA_{ds}^{m}C_{ds}T_{ds}T_{ds}T_{ds}A_{ds}T_{ds}T_{ds}G_{ds}A_{ds}GₖₒTₖₒTₖ- | 0.0118 | 53 |
| | HPPO-GalNAc | | |
| 1525922 | AₖₛGₖₛ^{m}CₖₒA_{ds}^{m}C_{ds}T_{ds}T_{ds}T_{ds}A_{ds}T_{ds}T_{ds}G_{ds}A_{do}GₖₛTₖₛTₖ- | 0.0135 | 53 |
| | HPPO-GalNAc | | |
| 1525923 | AₖₛGₖₛ^{m}C_{kz}A_{ds}^{m}C_{ds}T_{ds}T_{ds}T_{ds}A_{ds}T_{ds}T_{ds}G_{ds}A_{dz}GₖₛTₖₛTₖ- | 0.0155 | 53 |
| | HPPO-GalNAc | | |
| 1525924 | AₖₛGₖₛ^{m}CₖₛA_{dz}^{m}C_{dz}T_{dz}T_{dz}T_{ds}A_{ds}T_{ds}T_{ds}G_{ds}A_{ds}GₖₛTₖₛTₖ- | 0.0111 | 53 |
| | HPPO-GalNAc | | |
| 1525925 | AₖₛGₖₒ^{m}CₖₒA_{dz}^{m}C_{dz}T_{dz}T_{dz}T_{ds}A_{ds}T_{ds}T_{ds}G_{ds}A_{ds}GₖₒTₖₛTₖ | 0.0118 | 53 |
| | HPPO-GalNAc | | |
| 1525919 | A_{kz}Gₖₒ^{m}CₖₒA_{ds}^{m}C_{ds}T_{ds}T_{ds}T_{ds}A_{ds}T_{ds}T_{ds}G_{ds}A_{ds}GₖₒT_{kz}Tₖ | 0.0292 | 53 |
| | HPPO-GalNAc | | |

A subscript “k” represents a cEt nucleoside, a subscript “d” represents a stereo-standard DNA nucleoside, a subscript “s” indicates a phosphorothioate internucleoside linkage, a subscript “o” indicates a phosphodiester internucleoside linkage, and a subscript “z” represents an internucleoside linkage of formula IX, which is a mesyl phosphoramidate linkage. Subscripts of nucleotides having an internucleoside linkage of formula IX are bold and underlined. A superscript “m” before a C represents a 5-methylcytosine.

### Treatment

C57/B6J mice (Jax) were divided into groups of four male mice each for modified oligonucleotide treatment. Each mouse received a single subcutaneous injection of modified oligonucleotide at a dose of 0.9 mg/kg. One group of four mice received subcutaneous injections of PBS. The PBS-injected group served as the control group to which oligonucleotide-treated groups were compared. Prior to the first dose, a tail bleed was performed to determine plasma FXII protein levels at baseline (BL). Tail bleeds were also performed at 48 h, 96 h, 7 days, 14 days, and 21 days following the dose.

### Protein Analysis

Mouse FXII protein levels in plasma were determined using a FXII ELISA kit (Molecular Innovations catalog number: MFXIIKT-TOT). The data is presented as percent change from baseline within each treatment group.

TABLE 49
Reduction of mouse FXII protein in plasma

| | FXII protein (% baseline) in plasma at indicated time | | | | | |
|---|---|---|---|---|---|---|
| | after injection | | | | | |
| Compound | Day 0 | 48 | 96 | | | |
| No. | (baseline) | hours | hours | 7 day | 14 day | 21 day |
| PBS | 100 | 168 | 257 | 137 | 138 | 118 |
| 1525915 | 100 | 139 | 93 | 23 | 31 | 67 |
| 1525921 | 100 | 90 | 58 | 23 | 38 | 59 |
| 1525920 | 100 | 119 | 101 | 24 | 29 | 65 |
| 1525922 | 100 | 70 | 45 | 18 | 27 | 41 |
| 1525923 | 100 | 89 | 92 | 35 | 48 | 102 |
| 1525924 | 100 | 88 | 54 | 29 | 43 | 58 |
| 1525925 | 100 | 90 | 52 | 20 | 16 | 35 |
| 1525919 | 100 | 95 | 61 | 21 | 29 | 46 |

### Example 27: Design and Activity of siRNA Complementary to Mouse FXII in a Single Dose Duration of Action Study

Design of siRNA

Double-stranded siRNA compounds were formed by annealing one antisense strand and one sense strand described below. siRNA antisense strands containing mesyl phosphoramidate internucleoside linkages were designed as described in the table below and synthesized as described above. Each antisense strand has the sequence (from 5′ to 3′) UAAAGCACUUUAUUGAGUUUCUG (SEQ ID NO: 55) or TAAAGCACUUUAUUGAGUUUCUG (SEQ ID NO: 56), wherein the sequence (from 5′ to 3′) of AAAGCACUUUAUUGAGUUUCUG (SEQ ID NO: 57) is 100% complementary to mouse FXII, the complement of GENBANK NC_000079.6 truncated from 55415001 to 55430000 (SEQ ID NO: 54), at position 12005 to 12026.

TABLE 50
Design of antisense strand of modified
oligonucleotides complementary to mouse FXII

| Compound | | SEQ ID |
|---|---|---|
| ID | Chemistry Notation (5′ to 3′) | NO. |
| 1523579 | U_{ys}A_{fs}A_{yo}A_{yo}G_{yo}C_{fo}A_{yo}C_{yo}U_{yo}U_{yo}U_{yo}A_{yo}U_{yo}U_{fo}G_{yo}A_{fo}G_{yo}U_{yo}U_{yo}U_{yo}C_{ys}U_{ys}G_{y} | 55 |
| 1525955 | U_{ys}A_{fs}A_{yo}A_{yo}G_{yo}C_{fo}A_{yo}C_{yo}U_{yo}U_{yo}U_{yo}A_{yo}U_{yo}U_{fo}G_{yo}A_{fo}G_{yo}U_{yo}U_{yo}U_{yo}C_{yz}U_{yz}G_{y} | 55 |
| 1525956 | U_{yz}A_{fs}A_{yo}A_{yo}G_{yo}C_{fo}A_{yo}C_{yo}U_{yo}U_{yo}U_{yo}A_{yo}U_{yo}U_{fo}G_{yo}A_{fo}G_{yo}U_{yo}U_{yo}U_{yo}C_{yz}U_{yz}G_{y} | 55 |
| 1525957 | U_{ys}A_{fz}A_{yo}A_{yo}G_{yo}C_{fo}A_{yo}C_{yo}U_{yo}U_{yo}U_{yo}A_{yo}U_{yo}U_{fo}G_{yo}A_{fo}G_{yo}U_{yo}U_{yo}U_{yo}C_{yz}U_{yz}G_{y} | 55 |
| 1525958 | U_{yz}A_{fo}A_{yo}A_{yo}G_{yo}C_{fo}A_{yo}C_{yo}U_{yo}U_{yo}U_{yo}A_{yo}U_{yo}U_{fo}G_{yo}A_{fo}G_{yo}U_{yo}U_{yo}U_{yo}C_{yz}U_{yz}G_{y} | 55 |
| 1525959 | TₑₛA_{fo}A_{yo}A_{yo}G_{yo}C_{fo}A_{yo}C_{yo}U_{yo}U_{yo}U_{yo}A_{yo}U_{yo}U_{fo}G_{yo}A_{fo}G_{yo}U_{yo}U_{yo}U_{yo}C_{yz}U_{yz}G_{y} | 55 |
| 1527076 | U_{ys}A_{fo}A_{yo}A_{yo}G_{yo}C_{fo}A_{yo}C_{yo}U_{yo}U_{yo}U_{yo}A_{yo}U_{yo}U_{fo}G_{yo}A_{fo}G_{yo}U_{yo}U_{yo}U_{yo}C_{yz}U_{yz}G_{y} | 55 |
| 1528437 | T_{ez}A_{fo}A_{yo}A_{yo}G_{yo}C_{fo}A_{yo}C_{yo}U_{yo}U_{yo}U_{yo}A_{yo}U_{yo}U_{fo}G_{yo}A_{fo}G_{yo}U_{yo}U_{yo}U_{yo}C_{yz}U_{yz}G_{y} | 56 |
| 1528438 | TₑₒA_{fz}A_{yo}A_{yo}G_{yo}C_{fo}A_{yo}C_{yo}U_{yo}U_{yo}U_{yo}A_{yo}U_{yo}U_{fo}G_{fo}A_{fo}G_{yo}U_{yo}U_{yo}U_{yo}C_{yz}U_{yz}G_{y} | 56 |
| 1526197 | vP-TₑₛA_{fs}A_{yo}A_{yo}G_{yo}C_{fo}A_{yo}C_{yo}U_{yo}U_{yo}U_{yo}A_{yo}U_{yo}U_{fo}G_{yo}A_{fo}G_{yo}U_{yo}U_{yo}U_{yo}C_{yz}U_{yz}G_{y} | 56 |
| 1528440 | z.TₑₛA_{fs}A_{yo}A_{yo}G_{yo}C_{fo}A_{yo}C_{yo}U_{yo}U_{yo}U_{yo}A_{yo}U_{yo}U_{fo}G_{yo}A_{fo}G_{yo}U_{yo}U_{yo}U_{yo}C_{yz}U_{yz}G_{y} | 56 |

In the table, above, a subscript “e” represents a 2′-MOE modified nucleoside, a subscript “y” represents a 2′-OMe modified nucleoside, a subscript “f” represents a 2′-F modified nucleoside, a subscript “s” indicates a phosphorothioate internucleoside linkage, a subscript “o” indicates a phosphodiester internucleoside linkage, and a subscript “z” represents an internucleoside linkage of formula IX, which is a mesyl phosphoramidate linkage. Subscripts of nucleotides having an internucleoside linkage of formula IX are bold and underlined. Compound No. 1526197 contains a vinyl phosphonate (vP) moiety on the 5′-end. Compound No. 1528440 contains a 5′-mesylphosphoramidate having formula XXII:

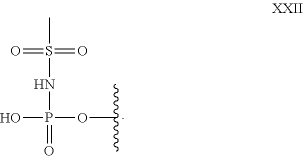 [SMILES: *OP(=O)(O)NS(C)(=O)=O]

TABLE 51
Design of sense strand of modified
oligonucleotides

| | | SEQ |
|---|---|---|
| Compound | | ID |
| ID | Chemistry Notation (5′ to 3′) | NO. |
| 1523578 | G_{ys}A_{ys}A_{yo}A_{yo}A_{yo}C_{yo}U_{yo}C_{fo}A_{yo}A_{fo}U_{fo}A_{fo}A_{yo}A_{yo}G_{yo} | 58 |
| | U_{yo}G_{yo}C_{yo}U_{yo}U_{yo}U_{yo}A_{y}-HPPO-GalNAc | |
| 1523580 | A_{ys}A_{ys}C_{yo}U_{yo}C_{yo}A_{yo}A_{fo}U_{yo}A_{fo}A_{fo}A_{fo}G_{yo}U_{yo}G_{yo}C_{yo} | 59 |
| | U_{yo}U_{yo}U_{yo}G_{yo}A_{yo}A_{y}-HPPO-GalNAc | |

In the table above, a subscript “f” represents a 2′-F modified nucleoside, a subscript “y” represents a 2′-OMe modified nucleoside, a subscript “s” indicates a phosphorothioate internucleoside linkage, and a subscript “o” represents a phosphodiester internucleoside linkage.

### In Vitro Activity

Activity of various siRNA formed by annealing one antisense strand and one sense strand described above was tested in primary mouse hepatocytes. Primary mouse hepatocytes cells were transfected with RNAiMAX formulated siRNA. Each modified oligonucleotide was transfected at a starting concentration of 200 nM with 5-fold serial dilutions for a total of 8 dilutions. After a treatment period of approximately 24 hours, RNA was isolated and RNA expression was analyzed via quantitative RTPCR using primer probe set RTS2959 (described herein above). FXII RNA levels were normalized to total GAPDH. Activity expressed as half maximal inhibitory concentration (IC₅₀) was calculated using the log (inhibitor) vs normalized response—Variable slope function in GraphPad Prism 7.

TABLE 52a
Reduction of mouse FXII protein

| siRNA Duplex | Antisense | Sense | |
|---|---|---|---|
| Compound | Compound | Compound | IC₅₀ |
| No. | No. | No. | (nM) |
| 1523582 | 1523579 | 1523578 | 0.0015 |
| 1523583 | 1523581 | 1523580 | 0.0076 |
| 1526140 | 1525955 | 1523578 | 0.0060 |
| 1526171 | 1525956 | 1523578 | 0.1304 |
| 1526182 | 1525957 | 1523578 | 0.0086 |
| 1526193 | 1525958 | 1523578 | 0.1171 |
| 1526194 | 1525959 | 1523578 | 0.0376 |
| 1527077 | 1527076 | 1523578 | 0.0053 |
| 1529977 | 1528437 | 1523578 | 0.1266 |
| 1529978 | 1528438 | 1523578 | 0.0292 |
| 1529980 | 1528440 | 1523578 | 0.00003 |
| 1526198 | 1526197 | 1523578 | 0.0014 |

### Treatment

C57/B6J mice (Jax) were divided into groups of four male mice each for treatment with siRNAs. Each mouse received a single subcutaneous injection of oligomeric duplex at a dose of 0.5 mg/kg. One group of four mice received subcutaneous injections of PBS. The PBS-injected group served as the control group to which oligonucleotide-treated groups were compared. Prior to the first dose, a tail bleed was performed to determine plasma FXII protein levels at baseline (BL). Tail bleeds were also performed at 48 h, 96 h, 7 days, 14 days, and 21 days following the dose.

### Protein Analysis

Mouse FXII protein levels in plasma were determined using a Molecular Innovations FXII ELISA kit (catalog number: MFXIIKT-TOT). The data is presented as percent change in protein, relative to PBS control.

TABLE 52b
Reduction of mouse FXII protein

| siRNA | Antisense | Sense | FXII protein (% baseline) in plasma at | | | | | |
|---|---|---|---|---|---|---|---|---|
| Duplex | Com- | Com- | indicated time after injection | | | | | |
| Compound | pound | pound | Day 0 | 48 | 96 | 7 | 14 | 21 |
| No. | No. | No. | (baseline) | hours | hours | day | day | day |
| PBS | N/A | N/A | 100 | 168 | 257 | 137 | 138 | 118 |
| 1523582 | 1523579 | 1523578 | 100 | 75 | 62 | 30 | 40 | 42 |
| 1523583 | 1523581 | 1523580 | 100 | 108 | 155 | 48 | 25 | 33 |
| 1526140 | 1525955 | 1523578 | 100 | 83 | 69 | 32 | 38 | 47 |
| 1526171 | 1525956 | 1523578 | 100 | 143 | 256 | 193 | 112 | 144 |
| 1526182 | 1525957 | 1523578 | 100 | 71 | 60 | 17 | 26 | 39 |
| 1526193 | 1525958 | 1523578 | 100 | 134 | 228 | 144 | 96 | 127 |
| 1526194 | 1525959 | 1523578 | 100 | 131 | 88 | 65 | 64 | 111 |
| 1527077 | 1527076 | 1523578 | 100 | 78 | 60 | 36 | 36 | 96 |
| 1529977 | 1528437 | 1523578 | 100 | 114 | 99 | 80 | 105 | 107 |
| 1529978 | 1528438 | 1523578 | 100 | 111 | 150 | 167 | 109 | 132 |
| 1526198 | 1526197 | 1523578 | 100 | 64 | 25 | 16 | 19 | 30 |

### Example 28: Design, Activity, and Tolerability of Modified Oligonucleotides with Mesyl Phosphoramidate Internucleoside Linkages in Combination with Stereo-Non-Standard Nucleosides

### Design of Modified Oligonucleotides

Modified oligonucleotides comprising multiple mesyl phosphoramidate internucleoside linkages (Formula IX) and phosphorothioate internucleoside linkages were synthesized and tested. Each of the modified oligonucleotides has the same nucleobase sequence, AGACTCTCGGTTCCGA (SEQ ID NO: 49), which is 100% complementary to mouse DNM2, GENBANK Accession No. NC_000075.6 truncated from 21422001 to 21511000 (SEQ ID NO: 47), at position 3046 to 3061. Each internucleoside linkage is either a phosphorothioate interucleoside linkage (“s”) or a mesyl phosphoramidate internucleoside linkage (“z”).

TABLE 53
Design of modified oligonucleotides with
mesyl phosphoramidate internucleoside linkages

| | | SEQ |
|---|---|---|
| Compound | | ID |
| ID | Chemistry Notation (5′ to 3′) | NO. |
| 694804 | AₖₛGₖₛAₖₛ^{m}C_{ds}T_{ds}^{m}C_{ds}T_{ds}^{m}C_{ds}G_{ds}G_{ds}T_{ds}T_{ds}^{m}C_{ds}^{m} | 49 |
| | CₖₛGₖₛAₖ | |
| 1537106 | AₖₛGₖₛAₖₛ^{m}C_{dz}T_{dz}^{m}C_{ds}T_{ds}^{m}C_{ds}G_{ds}G_{ds}T_{ds}T_{ds}^{m}C_{ds}^{m} | 49 |
| | CₖₛGₖₛAₖ | |
| 1537108 | AₖₛGₖₛAₖₛ^{m}C_{dz}U_{yz}^{m}C_{ds}T_{ds}^{m}C_{ds}G_{ds}G_{ds}T_{ds}T_{ds}^{m}C_{ds}^{m} | 48 |
| | CₖₛGₖₛAₖ | |
| 1537109 | AₖₛGₖₛAₖₛ^{m}C_{dz}T_{[}_{aDd}_{]}_{z}^{m}C_{ds}T_{ds}^{m}C_{ds}G_{ds}G_{ds}T_{ds}T_{ds}^{m}C_{ds}^{m} | 49 |
| | CₖₛGₖₛAₖ | |
| 1537110 | AₖₛGₖₛAₖₛ^{m}C_{dz}T_{[}_{dx}_{]}_{z}^{m}C_{ds}T_{ds}^{m}C_{ds}G_{ds}G_{ds}T_{ds}T_{ds}^{m}C_{ds}^{m} | 49 |
| | CₖₛGₖₛAₖ | |
| 1537111 | AₖₛGₖₛAₖₛ^{m}C_{dz}T_{[}_{bLd}_{]}_{z}^{m}C_{ds}T_{ds}^{m}C_{ds}G_{ds}G_{ds}T_{ds}T_{ds}^{m}C_{ds}^{m} | 49 |
| | CₖₛGₖₛAₖ | |
| 1537112 | AₖₛGₖₛAₖₛ^{m}C_{dz}T_{[}_{aLd}_{]}_{z}^{m}C_{ds}T_{ds}^{m}C_{ds}G_{ds}G_{ds}T_{ds}T_{ds}^{m}C_{ds}^{m} | 49 |
| | CₖₛGₖₛAₖ | |
| 1537113 | AₖₛGₖₛAₖₛ^{m}C_{dz}T_{[}_{aDdx}_{]}_{z}^{m}C_{ds}T_{ds}^{m}C_{ds}G_{ds}G_{ds}T_{ds}T_{ds}^{m} | 49 |
| | C_{ds}^{m}CₖₛGₖₛAₖ | |
| 1537114 | AₖₛGₖₛAₖₛ^{m}C_{dz}T_{[}_{bLdx}_{]}_{z}^{m}C_{ds}T_{ds}^{m}C_{ds}G_{ds}G_{ds}T_{ds}T_{ds}^{m} | 49 |
| | C_{ds}^{m}CₖₛGₖₛAₖ | |
| 1537115 | AₖₛGₖₛAₖₛ^{m}C_{dz}T_{[}_{aLdx}_{]}_{z}^{m}C_{ds}T_{ds}^{m}C_{ds}G_{ds}G_{ds}T_{ds}T_{ds}^{m} | 49 |
| | C_{ds}^{m}CₖₛGₖₛAₖ | |

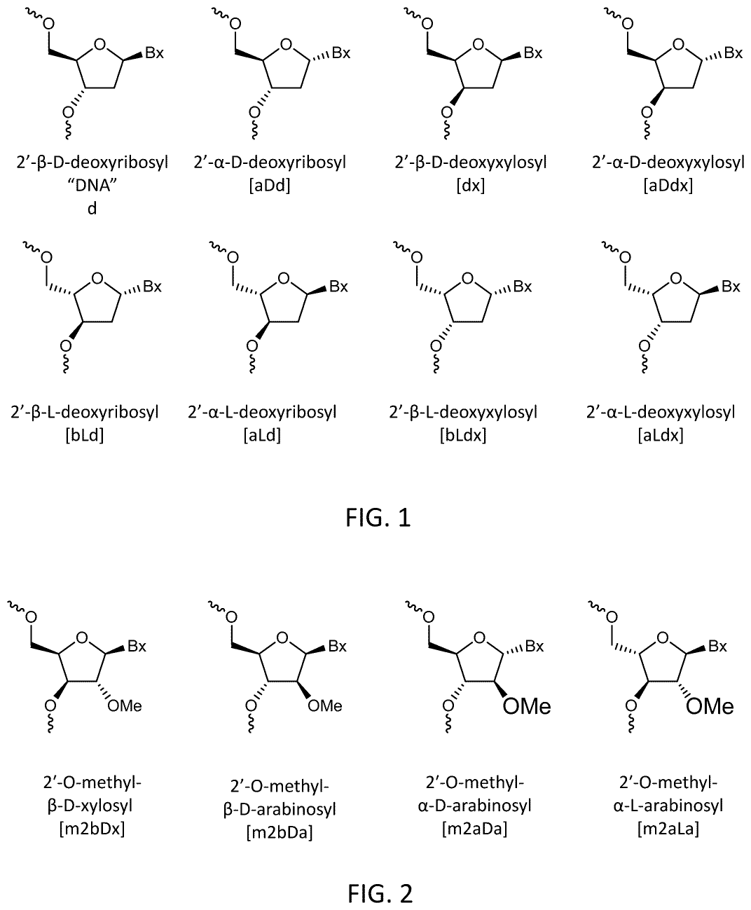

In the table above, a subscript “d” represents a stereo-standard DNA nucleoside, a subscript “y” represents a 2′-OMe modified nucleoside, a subscript “k” represents a cEt nucleoside, a subscript “s” indicates a phosphorothioate internucleoside linkage, and a subscript “z” represents an internucleoside linkage of formula IX, which is a mesyl phosphoramidate linkage. Subscripts of nucleotides having an internucleoside linkage of formula IX are bold and underlined. A superscript “m” before a C represents a 5-methylcytosine. A subscript “[bLd]” represents a 2′-β-L-deoxyribosyl sugar moiety, a subscript “[aDd]” represents a 2′-α-D-deoxyribosyl sugar moiety, a subscript “[aLd]” represents a 2′-α-deoxyribosyl sugar moiety, a subscript “[dx]” represents a 2′-β-D-deoxyxylosyl sugar moiety, a subscript “[bLdx]” represents a 2′-β-L-deoxyxylosyl sugar moiety, a subscript “[aDdx]” represents a 2′-α-D-deoxyxylosyl sugar moiety, a subscript “[aLdx]” represents a 2′-α-L-deoxyxylosyl sugar moiety (See FIG. 1)

### In Vitro Activity Assay

The modified oligonucleotides were tested for their ability to reduce target RNA in a series of experiments. Cultured mouse 3T3-L1 cells at a density of 20,000 cells per well were transfected using electroporation with modified oligonucleotides diluted to 20 μM, 7 μM, 2 μM, 0.7 μM, 0.3 μM, 0.1 μM, and 0.03 μM. After a treatment period of approximately 16 hours, RNA levels were measured using DNM2 primer probe set RTS36436 (forward sequence: AGAGGAGACCGAGCGAAT, SEQ ID NO: 50; reverse sequence: CATGGTTTGTGTTGATGTACGAC, SEQ ID NO: 51; probe sequence: CCTACATCAGGGAGCGAGAAGGGA, SEQ ID NO: 52). RNA levels for each target were normalized to total RNA content, as measured by RIBOGREEN®. Activity expressed as half maximal inhibitory concentration (IC₅₀) was calculated using the log (inhibitor) vs response (three parameter) function in GraphPad Prism 7.

### In Vitro Toxicity Assay

In vitro toxicity of modified oligonucleotides described above was determined as described in Example 3.

TABLE 54
In vitro activity and caspase activation by modified
oligonucleotides with mesyl phosphoramidate
internucleoside linkages in combination
with stereo-non-standard nucleosides

| Compound | Caspase Activation | IC₅₀ |
|---|---|---|
| ID | (% Mock) | (nM) |
| 694804 | 552 | 344 |
| 1537106 | 657 | 295 |
| 1537108 | 178 | 231 |
| 1537109 | 159 | 717 |
| 1537110 | 166 | 421 |
| 1537111 | 144 | 572 |
| 1537112 | 186 | 388 |
| 1537113 | 241 | 860 |
| 1537114 | 278 | 586 |
| 1537115 | 262 | 290 |

### Example 29: Design and Activity of siRNA with Mesyl Phosphoramidate Internucleoside Linkages to HPRT1 In Vitro

Design of siRNAs

Double-stranded siRNAs comprising modified oligonucleotides having mesyl phosphoramidate internucleoside linkages (Formula IX) and having either stereo-standard nucleosides or stereo-non-standard nucleosides were synthesized and tested. Each internucleoside linkage is either a phosphorothioate internucleoside linkage (“s”), a phosphodiester internucleoside linkage (“o”), or a mesyl phosphoramidate internucleoside linkage (“z”).

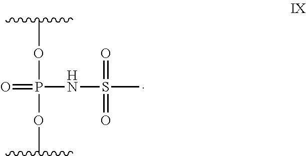 [SMILES: *OP(=O)(NS(C)(=O)=O)O*]

Each antisense strand has either the sequence (from 5′ to 3′): TUAAAAUCUACAGUCAUAGGATT (SEQ ID NO: 60) or UUAAAAUCUACAGUCAUAGGATT (SEQ ID NO: 61), wherein the sequence (from 5′ to 3′) UAAAAUCUACAGUCAUAGG^{A} (SEQ ID NO: 62) is 100% complementary to GenBank Accession No. NM_000194.2 (SEQ ID NO: 22) from 446 to 465, and each antisense strand has a 5′-phosphate.

The sense strand (Compound ID: 1505889) has the chemical notation (5′ to 3′): U_{ys}C_{ys}C_{yo}U_{yo}A_{yo}U_{yo}G_{fo}A_{yo}C_{fo}U_{fo}G_{fo}U_{yo}A_{yo}G_{yo}A_{yo}U_{yo}U_{yo}U_{yo}U_{ys}A_{ys}U_{y} (SEQ ID NO: 23), wherein a subscript “f” represents a 2′-F modified nucleoside, a subscript “y” represents a 2′-OMe modified nucleoside, a subscript “s” indicates a phosphorothioate internucleoside linkage, and a subscript “o” represents a phosphodiester internucleoside linkage.

TABLE 55
Design of antisense strand modified oligonucleotides
targeted to HPRT1 containing mesyl phosphoramidate
internucleoside linkages

| Compound | | SEQ ID |
|---|---|---|
| ID | Chemistry Notation (5′ to 3′) | NO. |
| 1512935 | P.T_{ys}U_{fs}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 60 |
| 1534483 | p.U_{ys}U_{fs}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 61 |
| 1534484 | p.T_{yz}U_{fo}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 60 |
| 1534485 | p.U_{yz}U_{fo}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 61 |
| 1534486 | P.U_{fz}U_{fo}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 61 |
| 1534487 | p.U_{[}_{f2Da}_{]}_{z}U_{fo}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 61 |
| 1534488 | P.U_{[f2bDx]o}U_{fz}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 61 |
| 1534489 | p.U_{[f2aDr]o}U_{fz}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 61 |
| 1534490 | P.U_{[f2aDa]o}U_{fz}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 61 |
| 1534491 | P.U_{[f2aDx]o}U_{fz}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 61 |
| 1534493 | p.U_{[f2aLr]o}U_{fz}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 61 |
| 1534494 | p.U_{[f2bLx]o}U_{fz}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 61 |
| 1534495 | p.U_{[f2aLa]o}U_{fz}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 61 |
| 1534496 | P.U_{[f2aLx]o}U_{fz}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 61 |
| 1534497 | P.U_{[f2bLr]o}U_{fz}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 61 |
| 1534492 | P.U_{[f2bLa]o}U_{fz}A_{yo}A_{yo}A_{yo}A_{fo}U_{yo}C_{fo}U_{fo}A_{yo}CycA_{yo}G_{yo}U_{fo}C_{yo}A_{fo}U_{yo}A_{yo}G_{yo}G_{yo}A_{ys}T_{ds}T_{d} | 61 |
| 1537089 | p.T_{[}_{m2bDx}_{]}_{z}U_{fo}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 60 |
| 1537090 | p.T_{[}_{m2bDa}_{]}_{z}U_{fo}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 60 |
| 1537091 | p.T_{[}_{m2aDa}_{]}_{z}U_{fo}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 60 |
| 1537092 | p.T_{[}_{m2aLa}_{]}_{z}U_{fo}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 60 |

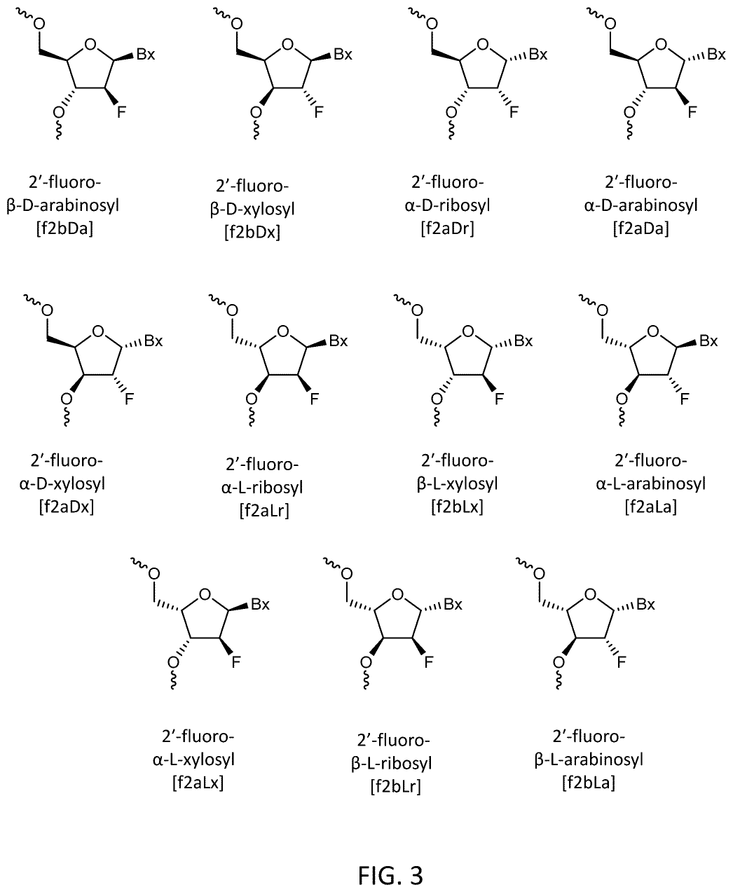

In the table above, a “p.” represents a 5′-phosphate, a subscript “d” represents a stereo-standard DNA nucleoside, a subscript “y” represents a 2′-OMe modified nucleoside, a subscript “f” represents a 2′-F modified nucleoside, a subscript “s” indicates a phosphorothioate internucleoside linkage, a subscript “o” indicates a phosphodiester internucleoside linkage, and a subscript “z” represents an internucleoside linkage of formula IX, which is a mesyl phosphoramidate linkage. Subscripts of nucleotides having an internucleoside linkage of formula IX are bold and underlined. A subscript “[f2bDa]” represents a 2′-fluoro-β-D-arabinosyl sugar moiety, a subscript “[f2bDx]” represents a 2′-fluoro-β-D-xylosyl sugar moiety, a subscript “[f2aDr]” represents a 2′-fluoro-α-D-ribosyl sugar moiety, a subscript “[f2aDa]” represents a 2′-fluoro-α-D-arabinosyl sugar moiety, a subscript “[f2aDx]” represents a 2′-fluoro-α-D-xylosyl sugar moiety, a subscript “[f2aLr]” represents a 2′-fluoro-α-ribosyl sugar moiety, a subscript “[f2bLx]” represents a 2′-fluoro-β-L-xylosyl sugar moiety, a subscript “[f2aLa]” represents a 2′-fluoro-α-arabinosyl sugar moiety, a subscript “[f2aLx]” represents a 2′-fluoro-α-xylosyl sugar moiety, a subscript “[f2bLr]” represents a 2′-fluoro-β-L-ribosyl sugar moiety, a subscript “[f2bLa]” represents a 2′-fluoro-β-L-arabinosyl sugar moiety, a subscript “[m2bDx]” represents a 2′-O-methyl-β-D-xylosyl sugar moiety, a subscript “[m2bDa]” represents a 2′-O-methyl-β-D-arabinosyl sugar moiety, a subscript “[m2aDa]” represents a 2′-O-methyl-α-D-arabinosyl sugar moiety, a subscript “[m2aLa]” represents a 2′-O-methyl-α-L-arabinosyl sugar moiety. (See FIG. 2 and FIG. 3)

### Activity Assay

Activity of various siRNA formed by annealing one antisense strand and one sense strand described above was tested in HeLa cells. HeLa cells were transfected with RNAiMAX formulated siRNA. Each siRNA compound was transfected at a starting concentration of 10 nM with 5-fold serial dilutions for a total of 8 dilutions. After a treatment period of approximately 6 hours, RNA was isolated and RNA expression was analyzed via quantitative RTPCR using primer probe set RTS35336 (forward sequence TTGTTGTAGGATATGCCCTTGA, SEQ ID NO: 63; reverse sequence: GCGATGTCAATAGGACTCCAG, SEQ ID NO: 64; probe sequence: AGCCTAAGATGAGAGTTCAAGTTGAGTTTGG, SEQ ID NO: 65). HPRT1 RNA levels were normalized to total RNA content, as measured by RIBOGREEN®. IC₅₀ values were calculated and are presented in the table below.

TABLE 56
Activity of siRNAs targeted to HPRT1 containing
mesyl phosphoramidate internucleoside linkages
and/or stereo-non-standard nucleosides

| Antisense | Sense | IC₅₀ |
|---|---|---|
| Strand | Strand | (nM) |
| 1455005 | 1505889 | 0.01 |
| 1512935 | 1505889 | 0.03 |
| 1534483 | 1505889 | 0.02 |
| 1534484 | 1505889 | 0.06 |
| 1534485 | 1505889 | 0.03 |
| 1534486 | 1505889 | 0.04 |
| 1534487 | 1505889 | 0.04 |
| 1534488 | 1505889 | 0.10 |
| 1534489 | 1505889 | 0.04 |
| 1534491 | 1505889 | 0.04 |
| 1534494 | 1505889 | 0.18 |
| 1534496 | 1505889 | 0.04 |
| 1534497 | 1505889 | 0.08 |
| 1534492 | 1505889 | 0.07 |
| 1537090 | 1505889 | 0.05 |
| 1537091 | 1505889 | 0.06 |
| 1537092 | 1505889 | 0.13 |

### Example 30: Evaluation of Proinflammatory Effects in BJAB Assay

Modified oligonucleotides targeting human CRP, human neurology Target X, human CXCL12, human oncology target Y, or human oncology target Z were tested for potential immunostimulatory properties in an in vitro human BJAB activation assay.

Immortalized human Burkitt lymphoma B cells, BJAB cells (DSMZ, Cat #ACC 757), were cultured in RPMI1640 medium containing 20% fetal bovine serum at 37° C. and 5% CO₂. Cells were maintained at the optimal recommended density of 0.5-0.7×10⁶ cells per milliliter. Cells were transferred to 50 mL conical Falcon tubes and centrifuged at 330 RCF for 5 minutes. Cells were resuspended at a concentration of 7.5×10⁵ cells per milliliter in RPMI culture medium. 50 mL per well of RPMI culture medium containing 200 U/mL penicillin and 200 mg/ml streptomycin was added to v-bottom tissue culture treated 96-well microplate. 50 μL of the cell suspension was added to the v-bottom tissue culture treated 96-well microplate. 11 μl of 10× concentrated modified oligonucleotides was then added to the plate and incubated for 24 hours at 37° C. and 5% CO₂.

The modified oligonucleotides were designed as described in the table below, wherein “d” represents a 2′-β-D-deoxyribosyl sugar moiety, “k” represents a cEt sugar moiety, and “e” represents a 2′-MOE sugar moiety. Each internucleoside linkage is either a phosphorothioate internucleoside linkage (“s”), aphosphodiester internucleoside linkage (“o”), a mesyl phosphoramidate internucleoside linkage of Formula IX (“z”), a mesyl phosphoramidate internucleoside linkage of Formula XI (“[XI]”), or a mesyl phosphoramidate internucleoside linkage of Formula XIII (“[XIII]”). Subscripts of nucleotides having a modified mesyl phosphoramidate internucleoside linkage of generic Formula XVII are bold and underlined. All cytosine residues are 5-methylcytosines. A nucleobase represented by N in the table below indicates A, G, T, or ^{m}C. Each oligonucleotide X (X1-X4) has the same sequence; each oligonucleotide Y (Y1-Y3) has the same sequence; and each oligonucleotide Z has the same sequence.

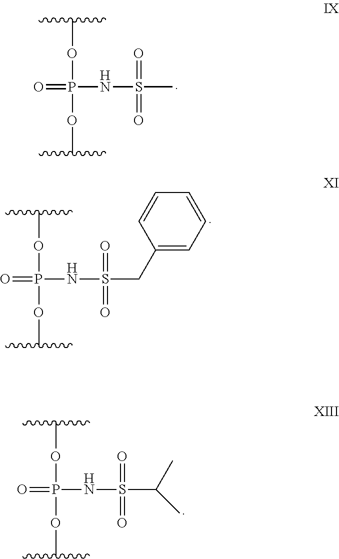 [SMILES: *OP(=O)(NS(C)(=O)=O)O* ; *OP(=O)(NS(=O)(=O)Cc1ccccc1)O* ; *OP(=O)(NS(=O)(=O)C(C)C)O*]

Compound Nos. 353512, 104838, 735746, and 785674 were added to the assay as standards. Compound No. 353512 is an internal standard known to be a high responder for CCL22 release in the assay. Compound No. 104838 is an internal standard known to be a non-responder in the assay (a negative control).

After incubation, total RNA was isolated. The amount of CCL22 mRNA was quantified using quantitative RTPCR. CCL22 PCR results were normalized to total GAPDH. Results are presented in the table below as log fold increase of CCL22, relative to untreated control.

TABLE 57
Design and BJAB inflammatory response of modified oligonucleotides
with mesyl phosphoramidate internucleoside linkages for BJAB assay

| | | | CCL22 | SEQ |
|---|---|---|---|---|
| Compound | | | log fold | ID |
| ID | Chemistry Notation (5′ to 3′) | Target | increase | NO. |
| 104838 | Gₑₛ^{m}CₑₛTₑₛGₑₛA_{ds}T_{ds}T_{ds}A_{ds}G_{ds}A_{ds} | Standard - low | 0.32 | 66 |
| | G_{ds}A_{ds}G_{ds}A_{ds}G_{ds}GₑₛTₑₛ^{m}Cₑₛ^{m}Cₑₛ^{m}Cₑ | | | |
| 353512 | Tₑₛ^{m}Cₑₛ^{m}Cₑₛ^{m}C_{ds}A_{ds}T_{ds}T_{ds}T_{ds}^{m}C_{ds}A_{ds} | Standard - high | 1.48 | 67 |
| | G_{ds}G_{ds}A_{ds}G_{ds}A_{ds}^{m}C_{ds}^{m}C_{ds}TₑₛGₑₛGₑ | | | |
| X1 | NₑₛNₑₒNₑₒNₑₒNₑₛN_{ds}N_{ds}N_{ds}N_{ds}N_{ds} | Target X | 2.38 | 70 |
| | N_{ds}N_{ds}N_{ds}N_{ds}N_{ds}NₑₒNₑₒNₑₛNₑₛNₑ | | | |
| X2 | N_{ez}N_{ez}NₑₒNₑₒNₑₛN_{ds}N_{ds}N_{ds}N_{ds}N_{ds} | Target X | 1.63 | 70 |
| | N_{ds}N_{ds}N_{ds}N_{ds}N_{ds}NₑₒNₑₒN_{ez}N_{ez}Nₑ | | | |
| X3 | NₑₛNₑₒNₑₒNₑₒNₑₛN_{dz}N_{dz}N_{dz}N_{ds}N_{ds} | Target X | 0.70 | 70 |
| | N_{ds}N_{ds}N_{ds}N_{ds}N_{ds}NₑₒNₑₒNₑₛNₑₛNₑ | | | |
| X4 | NₑₛNₑₒNₑₒNₑₒNₑₛN_{ds}N_{ds}N_{ds}N_{ds}N_{ds} | Target X | 0.95 | 70 |
| | N_{ds}N_{ds}N_{dz}N_{dz}N_{dz}NₑₒNₑₒNₑₛNₑₛNₑ | | | |
| 353512 | Tₑₛ^{m}Cₑₛ^{m}Cₑₛ^{m}C_{ds}A_{ds}T_{ds}T_{ds}T_{ds}^{m}C_{ds}A_{ds} | CRP | 1.40 | 69 |
| | G_{ds}G_{ds}A_{ds}G_{ds}A_{ds}^{m}C_{ds}^{m}C_{ds}TₑₛGₑₛGₑ | | | |
| 1523450 | Tₑₛ^{m}Cₑₛ^{m}Cₑₛ^{m}C_{dz}A_{dz}T_{dz}T_{ds}T_{ds}^{m}C_{ds}A_{ds} | CRP | 0.09 | 69 |
| | G_{ds}G_{ds}A_{ds}G_{ds}A_{ds}^{m}C_{ds}^{m}C_{ds}TₑₛGₑₛGₑ | | | |
| 1523451 | Tₑₛ^{m}Cₑₛ^{m}Cₑₛ^{m}C_{ds}A_{ds}T_{ds}T_{ds}T_{ds}^{m}C_{ds}A_{ds} | CRP | 0.57 | 69 |
| | G_{ds}G_{ds}A_{ds}G_{ds}A_{dz}^{m}C_{dz}^{m}C_{dz}TₑₛGₑₛGₑ | | | |
| Y1 | NₖₛNₖₛNₖₛN_{ds}N_{ds}N_{ds}N_{ds}N_{ds}N_{ds}N_{ds}N_{ds}N_{ds}N_{ds}NₖₛNₖₛNₖ | Target Y | 1.33 | 29 |
| Y2 | NₖₛNₖₛNₖN_{dz}N_{dz}N_{dz}N_{ds}N_{ds}N_{ds}N_{ds}N_{ds}N_{ds}N_{ds}NₖₛNₖₛNₖ | Target Y | 0.77 | 29 |
| Y3 | NₖₛNₖₛNₖₛN_{ds}N_{ds}N_{ds}N_{ds}N_{ds}N_{ds}N_{ds}N_{dz}N_{dz}N_{dz}NₖₛNₖₛNₖ | Target Y | 0.24 | 29 |
| Z1 | NₖₛNₖₛNₖₛN_{ds}N_{ds}N_{ds}N_{ds}N_{ds}N_{ds}N_{ds}N_{ds}N_{ds}N_{ds}NₖₛNₖₛNₖ | Target Z | 1.15 | 29 |
| Z2 | N_{dz}N_{dz}NₖₛN_{ds}N_{ds}N_{ds}N_{ds}N_{ds}N_{ds}N_{ds}N_{ds}N_{ds}N_{ds}N_{kz}N_{kz}Nₖ | Target Z | 0.03 | 29 |
| Z3 | NₖₛNₖₛNₖN_{dz}N_{dz}N_{dz}N_{ds}N_{ds}N_{ds}N_{ds}N_{ds}N_{ds}N_{ds}NₖₛNₖₛNₖ | Target Z | 0.56 | 29 |
| Z4 | NₖₛNₖₛNₖₛN_{ds}N_{ds}N_{ds}N_{ds}N_{ds}N_{ds}N_{ds}N_{dz}N_{dz}N_{dz}NₖₛNₖₛNₖ | Target Z | 0.05 | 29 |
| 558807 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | CXCL12 | 0.33 | 5 |
| 1375403 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛT_{kz}Aₖ | CXCL12 | 0.39 | 5 |
| 1375404 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}T_{kz}TₖₛAₖ | CXCL12 | 0.34 | 5 |
| 1375405 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{dz}TₖₛTₖₛAₖ | CXCL12 | 0.07 | 5 |
| 1375406 | Gₖₛ^{m}CₖₛAₖₛT_{ds}G_{ds}T_{ds}T_{ds}^{m}C_{ds}T_{ds}^{m}C_{ds}A_{ds}^{m}C_{dz}A_{ds}TₖₛTₖₛAₖ | CXCL12 | 0.23 | 5 |
| 1375407 | Gₖₛ^{m}CₖₛAₖₛT_{ds}G_{ds}T_{ds}T_{ds}^{m}C_{ds}T_{ds}^{m}C_{ds}A_{dz}^{m}C_{ds}A_{ds}TₖₛTₖₛAₖ | CXCL12 | 0.19 | 5 |
| 1375408 | Gₖₛ^{m}CₖₛAₖₛT_{ds}G_{ds}T_{ds}T_{ds}^{m}C_{ds}T_{ds}^{m}C_{dz}A_{ds}^{m}C_{ds}A_{ds}TₖₛTₖₛAₖ | CXCL12 | 0.16 | 5 |
| 1375409 | Gₖₛ^{m}CₖₛAₖₛT_{ds}G_{ds}T_{ds}T_{ds}^{m}C_{ds}T_{dz}^{m}C_{ds}A_{ds}^{m}C_{ds}A_{ds}TₖₛTₖₛAₖ | CXCL12 | 0.24 | 5 |
| 1375410 | Gₖₛ^{m}CₖₛAₖₛT_{ds}G_{ds}T_{ds}T_{ds}^{m}C_{dz}T_{ds}^{m}C_{ds}A_{ds}^{m}C_{ds}A_{ds}TₖₛTₖₛAₖ | CXCL12 | 0.33 | 5 |
| 1375411 | Gₖₛ^{m}CₖₛAₖₛT_{ds}G_{ds}T_{ds}T_{dz}^{m}C_{ds}T_{ds}^{m}C_{ds}A_{ds}^{m}C_{ds}A_{ds}TₖₛTₖₛAₖ | CXCL12 | 0.23 | 5 |
| 1375412 | Gₖₛ^{m}CₖₛAₖₛT_{ds}G_{ds}T_{dz}T_{ds}^{m}C_{ds}T_{ds}^{m}C_{ds}A_{ds}^{m}C_{ds}A_{ds}TₖₛTₖₛAₖ | CXCL12 | 0.37 | 5 |
| 1375413 | Gₖₛ^{m}CₖₛAₖₛT_{ds}G_{dz}T_{ds}T_{ds}^{m}C_{ds}T_{ds}^{m}C_{ds}A_{ds}^{m}C_{ds}A_{ds}TₖₛTₖₛAₖ | CXCL12 | 0.32 | 5 |
| 1375414 | Gₖₛ^{m}CₖₛAₖₛT_{dz}G_{ds}T_{ds}T_{ds}^{m}C_{ds}T_{ds}^{m}C_{ds}A_{ds}^{m}C_{ds}A_{ds}TₖₛTₖₛAₖ | CXCL12 | 0.22 | 5 |
| 1375415 | Gₖₛ^{m}CₖₛA_{kz}T_{ds}G_{ds}T_{ds}T_{ds}^{m}C_{ds}T_{ds}^{m}C_{ds}A_{ds}^{m}C_{ds}A_{ds}TₖₛTₖₛAₖ | CXCL12 | 0.55 | 5 |
| 1375416 | Gₖₛ^{m}C_{kz}AₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | CXCL12 | 0.29 | 5 |
| 1375417 | G_{kz}^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | CXCL12 | 0.21 | 5 |
| 1375418 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}T_{kz}T_{kz}Aₖ | CXCL12 | 0.25 | 5 |
| 1375419 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{dz}T_{kz}T_{kz}Aₖ | CXCL12 | 0.06 | 5 |
| 1375420 | Gₖₛ^{m}CₖₛAₖₛT_{ds}G_{ds}T_{ds}T_{ds}^{m}C_{ds}T_{ds}^{m}C_{ds}A_{ds}^{m}C_{dz}A_{dz}TₖₛTₖₛAₖ | CXCL12 | 0.17 | 5 |
| 1375421 | Gₖₛ^{m}CₖₛAₖₛT_{ds}G_{ds}T_{ds}T_{ds}^{m}C_{ds}T_{ds}^{m}C_{ds}A_{dz}^{m}C_{dz}A_{ds}TₖₛTₖₛAₖ | CXCL12 | 0.05 | 5 |
| 1375422 | Gₖₛ^{m}CₖₛAₖₛT_{ds}G_{ds}T_{ds}T_{ds}^{m}C_{ds}T_{ds}^{m}C_{dz}A_{dz}^{m}C_{ds}A_{ds}TₖₛTₖₛAₖ | CXCL12 | 0.13 | 5 |
| 1375423 | Gₖₛ^{m}CₖₛAₖₛT_{ds}G_{ds}T_{ds}T_{ds}^{m}C_{ds}T_{dz}^{m}C_{dz}A_{ds}^{m}C_{ds}A_{ds}TₖₛTₖₛAₖ | CXCL12 | 0.11 | 5 |
| 1375424 | Gₖₛ^{m}CₖₛAₖₛT_{ds}G_{ds}T_{ds}T_{ds}^{m}C_{dz}T_{dz}^{m}C_{ds}A_{ds}^{m}C_{ds}A_{ds}TₖₛTₖₛAₖ | CXCL12 | 0.18 | 5 |
| 1375425 | Gₖₛ^{m}CₖₛAₖₛT_{ds}G_{ds}T_{ds}T_{dz}^{m}C_{dz}T_{ds}^{m}C_{ds}A_{ds}^{m}C_{ds}A_{ds}TₖₛTₖₛAₖ | CXCL12 | 0.23 | 5 |
| 1375426 | Gₖₛ^{m}CₖₛAₖₛT_{ds}G_{ds}T_{dz}T_{dz}^{m}C_{ds}T_{ds}^{m}C_{ds}A_{ds}^{m}C_{ds}A_{ds}TₖₛTₖₛAₖ | CXCL12 | 0.65 | 5 |
| 1375427 | Gₖₛ^{m}CₖₛAₖₛT_{ds}G_{dz}T_{dz}T_{ds}^{m}C_{ds}T_{ds}^{m}C_{ds}A_{ds}^{m}C_{ds}A_{ds}TₖₛTₖₛAₖ | CXCL12 | 0.26 | 5 |
| 1375428 | Gₖₛ^{m}CₖₛAₖₛT_{dz}G_{dz}T_{ds}T_{ds}^{m}C_{ds}T_{ds}^{m}C_{ds}A_{ds}^{m}C_{ds}A_{ds}TₖₛTₖₛAₖ | CXCL12 | 0.15 | 5 |
| 1375429 | Gₖₛ^{m}CₖₛA_{kz}T_{dz}G_{ds}T_{ds}T_{ds}^{m}C_{ds}T_{ds}^{m}C_{ds}A_{ds}^{m}C_{ds}A_{ds}TₖₛTₖₛAₖ | CXCL12 | 0.42 | 5 |
| 1375430 | Gₖₛ^{m}C_{kz}A_{kz}T_{ds}G_{ds}T_{ds}T_{ds}^{m}C_{ds}T_{ds}^{m}C_{ds}A_{ds}^{m}C_{ds}A_{ds}TₖₛTₖₛAₖ | CXCL12 | 0.27 | 5 |
| 1375431 | G_{kz}^{m}C_{kz}AₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | CXCL12 | 0.20 | 5 |
| 1375432 | G_{kz}^{m}C_{kz}A_{kz}T_{dz}G_{dz}T_{dz}T_{dz}^{m}C_{dz}T_{dz}^{m}C_{dz}A_{dz}^{m}C_{dz}A_{dz}T_{kz}T_{kz}Aₖ | CXCL12 | 0.00 | 5 |
| 1378793 | Gₖₛ^{m}CₖₛA_{kz}T_{dz}G_{dz}T_{dz}T_{dz}^{m}C_{dz}T_{dz}^{m}C_{dz}A_{dz}^{m}C_{dz}A_{ds}TₖₛTₖₛAₖ | CXCL12 | −0.04 | 5 |
| 1378794 | G_{kz}^{m}C_{kz}AₖₛT_{ds}G_{ds}T_{ds}T_{ds}^{m}C_{ds}T_{ds}^{m}C_{ds}A_{ds}^{m}C_{ds}A_{dz}T_{kz}T_{kz}Aₖ | CXCL12 | 0.12 | 5 |
| 1386094 | Gₖₛ^{m}CₖₛA_{kz}T_{dz}G_{dz}T_{dz}T_{dz}^{m}C_{dz}T_{dz}^{m}C_{dz}A_{dz}^{m}C_{dz}A_{dz}TₖₛTₖₛAₖ | CXCL12 | −0.12 | 5 |
| 1386355 | Gₖₛ^{m}CₖₛAₖₛT_{dz}G_{dz}T_{dz}T_{dz}^{m}C_{dz}T_{dz}^{m}C_{dz}A_{dz}^{m}C_{dz}A_{ds}TₖₛTₖₛAₖ | CXCL12 | 0.01 | 5 |
| 1405434 | Gₖₛ^{m}CₖₛAₖₛT_{dz}G_{dz}T_{dz}TdsT_{ds}^{m}C_{ds}T_{ds}^{m}C_{ds}A_{ds}^{m}C_{ds}A_{ds}TₖₛTₖₛAₖ | CXCL12 | 0.08 | 5 |
| 1405435 | Gₖₛ^{m}CₖₛAₖₛT_{ds}G_{dz}T_{dz}T_{dz}^{m}C_{ds}T_{ds}^{m}C_{ds}A_{ds}^{m}C_{ds}A_{ds}TₖₛTₖₛAₖ | CXCL12 | 0.21 | 5 |
| 1405436 | Gₖₛ^{m}CₖₛAₖₛT_{dz}G_{dz}T_{dz}T_{dz}^{m}C_{ds}T_{ds}^{m}C_{ds}A_{ds}^{m}C_{ds}A_{ds}TₖₛTₖₛAₖ | CXCL12 | 0.24 | 5 |
| 1427921 | Gₖₛ^{m}CₖₛAₖₛT_{d}_{[}_{XI}_{]}G_{d}_{[}_{XI}_{]}T_{ds}T_{ds}^{m}C_{ds}T_{ds}^{m}C_{ds}A_{ds}^{m}C_{ds}A_{ds}TₖₛTₖₛAₖ | CXCL12 | 0.19 | 5 |
| 1427922 | Gₖₛ^{m}CₖₛAₖₛT_{ds}G_{d}_{[}_{XI}_{]}T_{d}_{[}_{XI}_{]}T_{ds}^{m}C_{ds}T_{ds}^{m}C_{ds}A_{ds}^{m}C_{ds}A_{ds}TₖₛTₖₛAₖ | CXCL12 | 0.26 | 5 |
| 1427923 | Gₖₛ^{m}CₖₛAₖₛT_{ds}G_{ds}T_{d}_{[}_{XI}_{]}T_{d}_{[}_{XI}_{]}^{m}C_{ds}T_{ds}^{m}C_{ds}A_{ds}^{m}C_{ds}A_{ds}TₖₛTₖₛAₖ | CXCL12 | 0.44 | 5 |
| 1427924 | Gₖₛ^{m}CₖₛAₖₛT_{ds}G_{ds}T_{ds}T_{ds}^{m}C_{ds}T_{ds}^{m}C_{d}_{[}_{XI}_{]}A_{d}_{[}_{XI}_{]}^{m}C_{ds}A_{ds}TₖₛTₖₛAₖ | CXCL12 | 0.13 | 5 |
| 1429189 | Gₖₛ^{m}CₖₛAₖₛT_{d}_{[}_{XIII}_{]}G_{d}_{[}_{XIII}_{]}T_{ds}T_{ds}^{m}C_{ds}T_{ds}^{m}C_{ds}A_{ds}^{m}C_{ds}A_{ds}TₖₛTₖₛAₖ | CXCL12 | 0.26 | 5 |
| 1429190 | Gₖₛ^{m}CₖₛAₖₛT_{ds}G_{d}_{[}_{XIII}_{]}T_{d}_{[}_{XIII}_{]}T_{ds}^{m}C_{ds}T_{ds}^{m}C_{ds}A_{ds}^{m}C_{ds}A_{ds}TₖₛTₖₛAₖ | CXCL12 | 0.39 | 5 |
| 1429191 | Gₖₛ^{m}CₖₛAₖₛT_{ds}G_{ds}T_{d}_{[}_{XIII}_{]}T_{d} _{[}_{XIII}_{]}^{m}C_{ds}T_{ds}^{m}C_{ds}A_{ds}^{m}C_{ds}A_{ds}TₖₛTₖₛAₖ | CXCL12 | 0.34 | 5 |
| 1429192 | Gₖₛ^{m}CₖₛAₖₛT_{ds}G_{ds}T_{ds}T_{ds}^{m}C_{ds}T_{ds}^{m}C_{d}_{[}_{XIII}_{]}A_{d}_{[}_{XIII}_{]}^{m}C_{ds}A_{ds}TₖₛTₖₛAₖ | CXCL12 | 0.16 | 5 |
| 1437601 | Gₖₛ^{m}CₖₛAₖₛT_{ds}G_{ds}T_{ds}T_{ds}^{m}C_{ds}T_{dz}^{m}C_{dz}A_{dz}^{m}C_{dz}A_{dz}TₖₛTₖₛAₖ | CXCL12 | 0.00 | 5 |
| 1437602 | Gₖₛ^{m}CₖₛAₖₛT_{ds}G_{ds}T_{ds}T_{ds}^{m}C_{dz}T_{dz}^{m}C_{dz}A_{dz}^{m}C_{dz}A_{dz}TₖₛTₖₛAₖ | CXCL12 | −0.03 | 5 |
| 1437603 | Gₖₛ^{m}CₖₛAₖₛT_{ds}G_{ds}T_{ds}T_{dz}^{m}C_{dz}T_{dz}^{m}C_{dz}A_{dz}^{m}C_{dz}A_{dz}TₖₛTₖₛAₖ | CXCL12 | −0.08 | 5 |
| 1437604 | Gₖₛ^{m}CₖₛAₖₛT_{ds}G_{ds}T_{dz}T_{dz}^{m}C_{dz}T_{dz}^{m}C_{dz}A_{dz}^{m}C_{dz}A_{dz}TₖₛTₖₛAₖ | CXCL12 | −0.10 | 5 |
| 1437605 | Gₖₛ^{m}CₖₛAₖₛT_{ds}G_{dz}T_{dz}T_{dz}^{m}C_{dz}T_{dz}^{m}C_{dz}A_{dz}^{m}C_{dz}A_{dz}TₖₛTₖₛAₖ | CXCL12 | −0.09 | 5 |
| 1437606 | Gₖₛ^{m}CₖₛAₖₛT_{dz}G_{dz}T_{dz}T_{dz}^{m}C_{dz}T_{dz}^{m}C_{dz}A_{dz}^{m}C_{dz}A_{dz}TₖₛTₖₛAₖ | CXCL12 | −0.04 | 5 |
| 1441068 | Gₖₛ^{m}CₖₛA_{dz}T_{ds}G_{dz}T_{ds}T_{dz}^{m}C_{ds}T_{dz}^{m}C_{ds}A_{dz}^{m}C_{ds}A_{dz}TₖₛTₖₛAₖ | CXCL12 | −0.04 | 5 |
| 1441069 | Gₖₛ^{m}CₖₛAₖₛT_{dz}G_{ds}T_{dz}T_{ds}^{m}C_{dz}T_{ds}^{m}C_{dz}A_{ds}^{m}C_{dz}A_{ds}TₖₛTₖₛAₖ | CXCL12 | −0.03 | 5 |

In the table above, a subscript “d” represents a 2′-β-D-deoxyribosyl sugar moiety, “k” represents a cEt sugar moiety, and “e” represents a 2′-MOE sugar moiety. Each internucleoside linkage is either a phosphorothioate internucleoside linkage (“s”), a phosphodiester internucleoside linkage (“o”), a mesyl phosphoramidate internucleoside linkage of Formula IX (“z”), a mesyl phosphoramidate internucleoside linkage of Formula XI (“[XI]”), or a mesyl phosphoramidate internucleoside linkage of Formula XIII (“[XIII]”). Subscripts of nucleotides having a modified mesyl phosphoramidate internucleoside linkage of generic Formula XVII are bold and underlined. All cytosine residues are 5-methylcytosines. A nucleobase represented by N in the table above indicates A, G, T, or ^{m}C.

### Example 31: Exonuclease Stability of Modified Oligonucleotides with Mesyl Phosphoramidate Internucleoside Linkages

Oligonucleotides comprising mesyl phosphoramidate internucleoside linkages were synthesized using standard techniques or those described herein. Each oligonucleotide in the table below has the sequence TTTTTTTTTTTT (SEQ ID NO:71).

The oligonucleotides described below were incubated at 5 μM concentration in buffer with snake venom phosphodiesterase (SVPD, Sigma P4506, Lot #SLBV4179), a strong 3′-exonuclease, at the standard concentration of 0.5 mU/mL and at a higher concentration of 2 mU/mL. SVPD is commonly used to measure the stability of modified nucleosides (see, e.g., Antisense Drug Technology, Crooke S. T., Ed., CRC Press, 2008). Aliquots were removed at various time points and analyzed by MS-HPLC with an internal standard. Relative peak areas were plotted versus time and half-life was determined using GraphPad Prism. A longer half-life means the 3′-terminal nucleosides have increased resistance to the SVPD exonuclease.

The results in the table below show that modified oligonucleotides comprising mesyl phosphoramidate internucleoside linkages are more stable to exonuclease degradation than unmodified DNA, 2′-MOE, and LNA with phosphodiester linkages. Such compounds are also more stable to exonuclease degradation than PS-linked DNA, and adding a second mesyl phosphoramidate internucleoside linkage on the 3′ end increases stability even further.

TABLE 58
Exonuclease resistance of modified oligonucleotides
with mesyl phosphoramidate internucleoside linkages

| | | | | SEQ |
|---|---|---|---|---|
| | | SVPD | T_{1/2} | ID |
| Compound ID | Chemistry Notation (5′ to 3′) | (mU/mL) | (min) | NO. |
| 7157 | T_{do}T_{do}T_{do}T_{do}T_{do}T_{do}T_{do}T_{do}T_{do}T_{do}T_{do}T_{d} | 0.5 | 0.4 | 71 |
| 395421 | T_{do}T_{do}T_{do}T_{do}T_{do}T_{do}T_{do}T_{do}T_{do}T_{do}TₑₒTₑ | 0.5 | 7.1, 4.8 | 71 |
| 395422 | T_{do}T_{do}T_{do}T_{do}T_{do}T_{do}T_{do}T_{do}T_{do}T_{do}TₗₒTₗ | 0.5 | 27.8 | 71 |
| 1506055 | T_{do}T_{do}T_{do}T_{do}T_{do}T_{do}T_{do}T_{do}T_{do}T_{do}T_{ds}T_{d} | 0.5 | 46.8 | 71 |
| 1506055 | T_{do}T_{do}T_{do}T_{do}T_{do}T_{do}T_{do}T_{do}T_{do}T_{do}T_{ds}T_{d} | 2 | 8.9 | 71 |
| 1468868 | T_{do}T_{do}T_{do}T_{do}T_{do}T_{do}T_{do}T_{do}T_{do}T_{do}T_{dz}T_{d} | 2 | 29.1 | 71 |
| 1468869 | T_{do}T_{do}T_{do}T_{do}T_{do}T_{do}T_{do}T_{do}T_{do}T_{dz}T_{dz}T_{d} | 2 | 69.6 | 71 |
| 1515981 | T_{do}T_{do}T_{do}T_{do}T_{do}T_{do}T_{do}T_{do}T_{do}T_{do}T_{lz}Tₗ | 2 | 26.7 | 71 |

A subscript “d” indicates a nucleoside comprising an unmodified, 2′-β-D-deoxyribosyl sugar moiety. A subscript “e” indicates a 2′-MOE sugar moiety. A subscript “l” indicates an LNA. A subscript “o” indicates a phosphodiester internucleoside linkage. A subscript “s” indicates a phosphorothioate internucleoside linkage. A subscript “z” indicates an internucleoside linkage of formula IX, which is a mesyl phosphoramidate linkage. Subscripts of nucleotides having an internucleoside linkage of formula IX are bold and underlined.

### Example 32: Exonuclease Stability of siRNA Antisense Oligonucleotides with Mesyl Phosphoramidate Internucleoside Linkages

Oligonucleotides comprising mesyl phosphoramidate internucleoside linkages were synthesized using standard techniques or those described herein. Each oligonucleotide in the table below has the sequence

(SEQ ID NO: 21)
AUAAAAUCUACAGUCAUAGGAAU.

### SVPD Assay

Selected oligonucleotides described below were tested in a 3′-exonuclease assay. Modified oligonucleotides were incubated at 5 μM concentration in buffer with snake venom phosphodiesterase (SVPD, Sigma P4506, Lot #SLBV4179), a strong 3′-exonuclease, at the standard concentration of 2 mU/mL. SVPD is commonly used to measure the stability of modified nucleosides (see, e.g., Antisense Drug Technology, Crooke S.T., Ed., CRC Press, 2008). Aliquots were removed at various time points and analyzed by MS-HPLC with an internal standard. Relative peak areas were plotted versus time and half-life was determined using GraphPad Prism. A longer half-life means the 3′-terminal nucleosides have increased resistance to the SVPD exonuclease.

### BSPDII Assay

Selected oligonucleotides described below were tested in a 5′-exonuclease assay. Modified oligonucleotides were first incubated with 100units/mL alkaline phosphatase (AP, Sigma P7923, Lot SLCB86083) in Tris-HCl buffer at pH 8.5 for 30 minutes, until the reaction was complete by MS-HPLC. The pH was adjusted to 6.5 and oligonucleotides were incubated with 5 mU/mL or 10 mU/mL bovine spleen phosphodiesterase II (BSPDII) (see Bernardi, A. and G. Bernardi, “Studies on acid hydrolases: IV. Isolation and characterization of spleen exonuclease.” Biochimica et Biophysica Acta-Nucleic Acids and Protein Synthesis 155(2): 360-370, 1968).

Aliquots were removed at various time points and analyzed by MS-HPLC with an internal standard. Relative peak areas were plotted versus time and half-life was determined using GraphPad Prism. A longer half-life means the 5′-terminal nucleosides have increased resistance to the PD II exonuclease.

Assay for Tritosome Stability of siRNA Antisense Oligonucleotides

Antisense oligonucleotides having modification patterns suitable for RNAi were investigated for tritosome stability in rat tritosomes. Modified oligonucleotides were incubated at 5 mM for 0 and 48 hours in 20% rat tritosomes in pH 4.5 acetate buffer. Samples were extracted utilizing standard protocols (Chappell, A. E., et al. (2020). “Mechanisms of palmitic acid-conjugated antisense oligonucleotide distribution in mice.” Nucleic Acids Res 48(8): 4382-4395) and analyzed by MS-HPLC with an internal standard. Relative peak areas for 0 and 48 h time points were determined and percent of full length modified oligonucleotide was calculated. Oligonucleotides lacking the 5′-terminal phosphate are included as “full length” due to rapid removal of this moiety.

Assay for Plasma Stability of siRNA Antisense Oligonucleotides

Antisense oligonucleotides having modification patterns suitable for RNAi were investigated for plasma stability in fresh mouse serum. Modified oligonucleotides were incubated for 0 and 24 hours in 50% fresh mouse serum. Samples were extracted utilizing standard protocols (Chappell, A. E., et al. (2020). “Mechanisms of palmitic acid-conjugated antisense oligonucleotide distribution in mice.” Nucleic Acids Res 48(8): 4382-4395) and analyzed by MS-HPLC with an internal standard. Relative peak areas for 0 and 24 h time points were determined and % intact modified oligonucleotide calculated.

### Assay for Alkaline Phosphatase Enzyme Stability

Antisense oligonucleotides having modification patterns suitable for RNAi were investigated for phosphatase stability utilizing alkaline phosphatase from bovine intestinal mucosa (AP, Sigma P7923, Lot SLCB86083). Modified oligonucleotides were incubated in Tris-HCl buffer at pH 8.5 containing 100 units/mL alkaline phosphatase (AP, Sigma P7923, Lot SLCB86083). Aliquots were removed at 30 minutes and analyzed by HPLC-MS. The 5′-terminal phosphate groups were removed at this time point for oligonucleotides 1337111, 1405420 and 1405428 while the 5′-terminal mesyl phosphoramidate group of 1527118 was still intact. 24- and 48-hour time points were taken for this compound and HPLC-MS analysis revealed that the mesyl phosphoramidate group was still present.

TABLE 59
Design of siRNA antisense oligonucleotides
with mesyl phosphoramidate internucleoside linkages

| | | SEQ |
|---|---|---|
| Compound | | ID |
| ID | Chemistry Notation (5′ to 3′) | NO. |
| 1527119 | p.A_{yo}U_{fo}A_{yo}A_{fo}A_{yo}A_{fo}U_{yo}C_{fo}U_{yo}A_{fo}C_{yo}A_{fo}G_{yo}U_{fo}C_{yo}A_{fo}U_{yo}A_{fo}G_{yo}G_{fo}A_{yo}A_{fo}U_{y} | 22 |
| 1073762 | p.A_{yo}U_{fo}A_{yo}A_{fo}A_{yo}A_{fo}U_{yo}C_{fo}U_{yo}A_{fo}C_{yo}A_{fo}G_{yo}U_{fo}C_{yo}A_{fo}U_{yo}A_{fo}G_{yo}G_{fo}A_{ys}A_{fs}U_{y} | 22 |
| 1337111 | p.A_{ys}U_{fs}A_{yo}A_{fo}A_{yo}A_{fo}U_{yo}C_{fo}U_{yo}A_{fo}C_{yo}A_{fo}G_{yo}U_{fo}C_{yo}A_{fo}U_{yo}A_{fo}G_{yo}G_{fo}A_{ys}A_{fs}U_{y} | 22 |
| 1405420 | p.A_{yo}U_{fo}A_{yo}A_{fo}A_{yo}A_{fo}U_{yo}C_{fo}U_{yo}A_{fo}C_{yo}A_{fo}G_{yo}U_{fo}C_{yo}A_{fo}U_{yo}A_{fo}G_{yo}G_{fo}A_{yz}A_{fz}U_{y} | 22 |
| 1405427 | p.A_{yz}A_{fz}A_{yo}A_{fo}A_{yo}A_{fo}U_{yo}C_{fo}U_{yo}A_{fo}C_{yo}A_{fo}G_{yo}U_{fo}C_{yo}A_{fo}U_{yo}A_{fo}G_{yo}G_{fo}A_{yo}A_{fo}U_{y} | 22 |
| 1405428 | p.A_{yz}A_{fz}A_{yo}A_{fo}A_{yo}A_{fo}U_{yo}C_{fo}C_{yo}A_{fo}C_{yo}A_{fo}G_{yo}U_{fo}C_{yo}A_{fo}U_{yo}A_{fo}G_{yo}G_{fo}A_{yz}A_{fz}U_{y} | 22 |

A subscript “y” represents a 2′-OMe modified nucleoside, a subscript “f” represents a 2′-F modified nucleoside, a subscript “s” indicates a phosphorothioate internucleoside linkage, a subscript “o” indicates a phosphodiester internucleoside linkage, and a subscript “z” represents an internucleoside linkage of formula IX, which is a mesyl phosphoramidate linkage. Subscripts of nucleotides having an internucleoside linkage of formula IX are bold and underlined. A superscript “m” before a C represents a 5-methylcytosine.

TABLE 60
Nuclease stability of siRNA antisense oligonucleotides
with mesyl phosphoramidate internucleoside linkages

| | SVPD | | | | | | |
|---|---|---|---|---|---|---|---|
| | Assay | BSPII Assay (5′) | | Tritosome | Plasma | AP Assay | |
| | (3′) | T½ (min) | | Stability | Stability | Time to | |
| Compound | T½ | T½ | Amount | % full | % full | remove | |
| ID | (min) | (min) | (mU/mL) | length | length | 5′-moiety | |
| 1527119 | 0.3 | n.d. | N/A | 0 | 0 | >48 | hours |
| 1073762 | 4.2 | n.d. | N/A | 0 | 28 | n.d. | |
| 1337111 | 5.5 | 29.7 | 10 | 55 | 28 | <30 | min |
| 1405420 | 12.8 | 2.7 | 5 | 0 | 58 | <30 | min |
| 1405427 | 0.3 | n.d. | N/A | 0 | 0 | n.d. | |
| 1405428 | 9.5 | 42.6 | 10 | 100 | 44 | <30 | min |

### Example 33: Design, Activity, and Tolerability of Modified Oligonucleotides with Mesyl Phosphoramidate Internucleoside Linkages In Vivo

### Design of Modified Oligonucleotides

Modified oligonucleotides comprising multiple mesyl phosphoramidate internucleoside linkages (Formula IX) and phosphorothioate intemucleoside linkages were designed, synthesized and tested. The modified oligonucleotides are each 5-10-5 MOE gapmers with a sugar motif of: eeeeeddddddddddeeeee, where “e” represents a 2′-MOE modified sugar moiety, and “d” represents a 2′-β-D-deoxyribosyl sugar moiety. Each of the modified oligonucleotides has the same nucleobase sequence, GCCAGGCTGGTTATGACTCA (SEQ ID NO: 72), which is 100% complementary to the complement of mouse Malatl, GENBANK Accession No. NC 000085.6 truncated from 5793001 to 5806000 (SEQ TD NO: 73), at position 6668 to 6687. Each internucleoside linkage is either a phosphorothioate internucleoside linkage (“s”), a phosphodiester intemucleoside linkage (“o”), or a mesyl phosphoramidate internucleoside linkage (“z”).

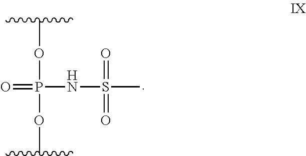 [SMILES: *OP(=O)(NS(C)(=O)=O)O*]

TABLE 61
Design of modified oligonucleotides with
mesyl phosphoramidate internucleoside linkages

| | | SEQ |
|---|---|---|
| Compound | | ID |
| ID | Chemistry Notation (5′ to 3′) | NO. |
| 602056 | Gₑₛ^{m}Cₑₛ^{m}CₑₛAₑₛGₑₛG_{ds}^{m}C_{ds}T_{ds}G_{ds}G_{ds}T_{ds}T_{ds}A_{ds}T_{ds}G_{ds}Aₑₛ^{m}CₑₛTₑₛ^{m}CₑₛAₑ | 72 |
| 626112 | Gₑₛ^{m}Cₑₒ^{m}CₑₒAₑₒGₑₒG_{ds}^{m}C_{ds}T_{ds}G_{ds}G_{ds}T_{ds}T_{ds}A_{ds}T_{ds}G_{ds}Aₑₒ^{m}CₑₒTₑₛ^{m}CₑₛAₑ | 72 |
| 1454990 | Gₑₒ^{m}Cₑₒ^{m}CₑₒAₑₒGₑₒG_{ds}^{m}C_{ds}T_{ds}G_{ds}G_{ds}T_{ds}T_{ds}A_{ds}T_{ds}G_{ds}Aₑₒ^{m}CₑₒTₑₒ^{m}CₑₒAₑ | 72 |
| 1469248 | Gₑₛ^{m}Cₑₒ^{m}CₑₒAₑₒGₑₒG_{ds}^{m}C_{ds}T_{ds}G_{ds}G_{ds}T_{ds}T_{ds}A_{ds}T_{ds}G_{ds}Aₑₒ^{m}CₑₒTₑₒ^{m}CₑₛAₑ | 72 |
| 1515340 | Gₑₛ^{m}Cₑₛ^{m}CₑₛAₑₛG_{ez}G_{ds}^{m}C_{ds}T_{ds}G_{ds}G_{ds}T_{ds}T_{ds}A_{ds}T_{ds}G_{dz}Aₑₛ^{m}CₑₛTₑₛ^{m}CₑₛAₑ | 72 |
| 1515341 | G_{ez}^{m}Cₑₛ^{m}CₑₛAₑₛGₑₛG_{ds}^{m}C_{ds}T_{ds}G_{ds}G_{ds}T_{ds}T_{ds}A_{ds}T_{ds}G_{ds}Aₑₛ^{m}CₑₛT_{ez}^{m}C_{ez}Aₑ | 72 |
| 1515342 | Gₑₛ^{m}Cₑₛ^{m}CₑₛAₑₛGₑₒG_{ds}^{m}C_{ds}T_{ds}G_{ds}G_{ds}T_{ds}T_{ds}A_{ds}T_{ds}G_{do}Aₑₛ^{m}CₑₛTₑₛ^{m}CₑₛAₑ | 72 |
| 1515344 | Gₑₛ^{m}Cₑₛ^{m}CₑₛAₑₛGₑₛG_{ds}^{m}C_{do}T_{ds}G_{ds}G_{ds}T_{ds}T_{ds}A_{ds}T_{ds}G_{ds}Aₑₛ^{m}CₑₛTₑₛ^{m}CₑₛAₑ | 72 |
| 1515346 | Gₑₛ^{m}Cₑₛ^{m}CₑₛAₑₛGₑₛG_{dz}^{m}C_{dz}T_{dz}G_{ds}G_{ds}T_{ds}T_{ds}A_{ds}T_{ds}G_{ds}Aₑₛ^{m}CₑₛTₑₛ^{m}CₑₛAₑ | 72 |
| 1515347 | Gₑₛ^{m}Cₑₛ^{m}CₑₛAₑₛG_{dz}^{m}C_{dz}T_{dz}G_{dz}G_{ds}T_{ds}T_{ds}A_{ds}T_{ds}G_{ds}Aₑₛ^{m}CₑₛTₑₛ^{m}CₑₛAₑ | 72 |
| 1515348 | Gₑₛ^{m}Cₑₛ^{m}CₑₛAₑₛGₑG_{dz}^{m}C_{dz}T_{dz}G_{dz}G_{dz}T_{ds}T_{ds}A_{ds}T_{ds}G_{ds}Aₑₛ^{m}CₑₛTₑₛ^{m}CₑₛAₑ | 72 |
| 1515350 | G_{ez}^{m}Cₑₒ^{m}CₑₒAₑₒGₑₒG_{ds}^{m}C_{ds}T_{ds}G_{ds}G_{ds}T_{ds}T_{ds}A_{ds}T_{ds}G_{ds}Aₑₒ^{m}CₑₒT_{ez}^{m}C_{ez}Aₑ | 72 |
| 1515355 | Gₑₛ^{m}Cₑₒ^{m}CₑₒAₑₒGₑₒG_{dz}^{m}C_{dz}T_{dz}G_{dz}G_{ds}T_{ds}T_{ds}A_{ds}T_{ds}G_{dz}Aₑₒ^{m}CₑₒTₑₛ^{m}CₑₛAₑ | 72 |
| 1524739 | G_{ez}^{m}Cₑₒ^{m}CₑₒAₑₒGₑₒG_{ds}^{m}C_{ds}T_{ds}G_{ds}G_{ds}T_{ds}T_{ds}A_{ds}T_{ds}G_{ds}Aₑₒ^{m}CₑₒTₑₒ^{m}C_{ez}Aₑ | 72 |
| 1524740 | G_{ez}^{m}Cₑₒ^{m}CₑₒAₑₒGₑₒG_{dz}^{m}C_{ds}T_{ds}G_{ds}G_{ds}T_{ds}T_{ds}A_{ds}T_{ds}G_{ds}Aₑₒ^{m}CₑₒTₑₒ^{m}C_{ez}Aₑ | 72 |
| 1524741 | G_{ez}z^{m}Cₑₒ^{m}CₑₒAₑₒGₑₒG_{dz}^{m}C_{dz}T_{ds}G_{ds}G_{ds}T_{ds}T_{ds}A_{ds}T_{ds}G_{ds}Aₑₒ^{m}CₑₒTₑₒ^{m}C_{ez}Aₑ | 72 |
| 1524742 | G_{ez}^{m}Cₑₒ^{m}CₑₒAₑₒGₑₒG_{dz}^{m}C_{dz}T_{dz}G_{ds}G_{ds}T_{ds}T_{ds}A_{ds}T_{ds}G_{ds}Aₑₒ^{m}CₑₒTₑₒ^{m}C_{ez}Aₑ | 72 |
| 1524743 | G_{ez}^{m}Cₑₒ^{m}CₑₒAₑₒGₑₒG_{dz}^{m}C_{dz}T_{dz}G_{dz}G_{ds}T_{ds}T_{ds}A_{ds}T_{ds}G_{ds}Aₑₒ^{m}CₑₒTₑₒ^{m}C_{ez}Aₑ | 72 |
| 1524744 | G_{ez}^{m}Cₑₒ^{m}CₑₒAₑₒGₑₒG_{dz}^{m}C_{dz}T_{dz}G_{dz}G_{dz}T_{ds}T_{ds}A_{ds}T_{ds}G_{ds}Aₑₒ^{m}CₑₒTₑₒ^{m}C_{ez}Aₑ | 72 |
| 1524745 | G_{ez}^{m}Cₑₒ^{m}CₑₒAₑₒGₑₒG_{dz}^{m}C_{dz}T_{dz}G_{dz}G_{dz}T_{dz}T_{ds}A_{ds}T_{ds}G_{ds}Aₑₒ^{m}CₑₒTₑₒ^{m}C_{ez}Aₑ | 72 |

A subscript “e” represents a 2′-MOE modified nucleoside, a subscript “d” represents a stereo-standard DNA nucleoside, a subscript “s” indicates a phosphorothioate internucleoside linkage, subscript “o” indicates a phosphodiester internucleoside linkage, and a subscript “z” represents an internucleoside linkage of formula IX, which is a mesyl phosphoramidate linkage. Subscripts of nucleotides having an internucleoside linkage of formula IX are bold and underlined. A superscript “m” before a C represents a 5-methylcytosine.

### Activity in CNS

Oligonucleotides described above were tested in wild-type female C57/B16 mice to assess the activity of the oligonucleotides. Wild-type female C57/B16 mice each received a single ICV bolus of 30 μg of modified oligonucleotide listed in the table below. Each treatment group consisted of 4 mice. A group of 4 mice received PBS as a negative control. Twelve days post treatment, mice were sacrificed and RNA was extracted from cortical brain tissue and spinal cord for quantitative RTPCR analysis to measure the amount of Malatl RNA using mouse primer probe set RTS592 (forward sequence CGGATGAAGAGAGGCATGTTG, designated herein as SEQ ID NO: 74; reverse sequence TTGGCCACACCGTCCTTT, designated herein as SEQ ID NO: 75; probe sequence AGACCTGGGCAATGTGGCTGCTG, designated herein as SEQ ID NO: 76). Results are presented as percent mouse Malatl RNA relative to PBS control, adjusted to mouse Cyclophilin A RNA.

TABLE 62
In vivo CNS activity of modified
oligonucleotides complementary to Malat1

| | | MALAT1 |
|---|---|---|
| | MALAT1 | RNA |
| | RNA | Spinal |
| Compound | Cortex | Cord (% |
| ID | (% Control) | Control) |
| 602056 | 37 | 16 |
| 626112 | 40 | 26 |
| 1454990 | 93 | 68 |
| 1469248 | 53 | 34 |
| 1515340 | 45 | 11 |
| 1515341 | 48 | 17 |
| 1515342 | 56 | 21 |
| 1515344 | 91 | 64 |
| 1515346 | 49 | 19 |
| 1515347 | 51 | 25 |
| 1515348 | 45 | 22 |
| 1515350 | 63 | 38 |
| 1515355 | 78 | 46 |
| 1524739 | 56 | 36 |
| 1524740 | 66 | 29 |
| 1524741 | 69 | 31 |
| 1524742 | 76 | 45 |
| 1524743 | 69 | 48 |
| 1524744 | 72 | 58 |
| 1524745 | 74 | 58 |

### Example 34: Activity and Tolerability of siRNA with Mesyl Phosphoramidate Internucleoside Linkages to HPRT1 In Vivo

siRNA

Double-stranded siRNA comprising modified oligonucleotides having mesyl phosphoramidate internucleoside linkages (Formula IX) in the antisense strand were synthesized and tested. Each internucleoside linkage is either a phosphorothioate internucleoside linkage (“s”), a phosphodiester internucleoside linkage (“o”), or a mesyl phosphoramidate internucleoside linkage (“z”).

Each antisense strand has the sequence AUAAAAUCUACAGUCAUAGGAAU (SEQ ID NO: 21) and is 100% complementary to GenBank NM_000194.2 (SEQ ID NO: 22) from 444 to 466, and each antisense strand has a 5′-phosphate. The sense strand, Compound No. 1448688, has the chemical notation U_{ys}C_{ys}C_{yo}U_{yo}A_{yo}U_{yo}G_{fo}A_{yo}C_{fo}U_{fo}G_{fo}U_{yo}A_{yo}G_{yo}A_{yo}U_{yo}U_{yo}U_{yo}U_{yo}A_{yo}U_{y}-THA-C7-GalNAc (SEQ ID NO: 23). The THA-C7-GalNAc conjugate is attached to the 3′-oxygen and has the structure below:

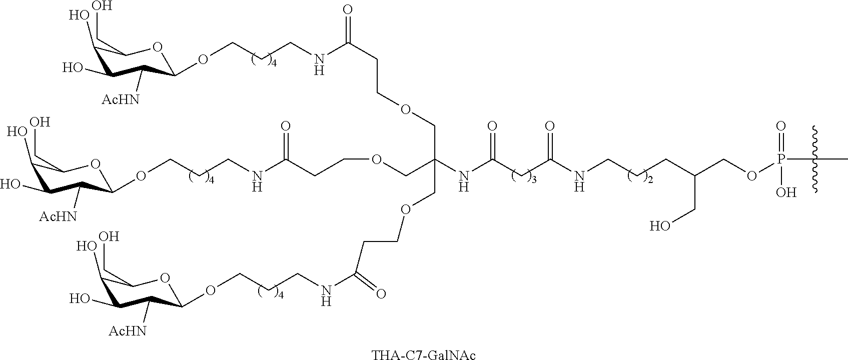

TABLE 63
Design of antisense strand modified oligonucleotides
targeted to HPRT1 containing mesyl phosphoramidate
internucleoside linkages

| | | SEQ |
|---|---|---|
| Compound | | ID |
| ID | Chemistry Notation (5′ to 3′) | NO. |
| 1337111 | p.A_{ys}U_{fs}A_{yo}A_{fo}A_{yo}A_{fo}U_{yo}C_{fo}U_{yo}A_{fo}C_{yo}A_{fo}G_{yo}U_{fo}C_{yo}A_{fo}U_{yo}A_{fo}G_{yo}G_{fo}A_{ys}A_{fs}U_{y} | 21 |
| 1405428 | p.A_{yz}U_{fz}A_{yo}A_{fo}A_{yo}A_{fo}U_{yo}C_{fo}U_{yo}A_{fo}C_{yo}A_{fo}G_{yo}U_{fo}C_{yo}A_{fo}U_{yo}A_{fo}G_{yo}G_{fo}A_{yz}A_{fz}U_{y} | 21 |
| 1465680 | p.A_{yz}U_{fs}A_{yo}A_{fo}A_{yo}A_{fo}U_{yo}C_{fo}U_{yo}A_{fo}C_{yo}A_{fo}G_{yo}U_{fo}C_{yo}A_{fo}U_{yo}A_{fo}G_{yo}G_{fo}A_{ys}A_{fs}U_{y} | 21 |
| 1465681 | p.A_{ys}U_{fz}_{yo}A_{fo}A_{yo}A_{fo}U_{yo}C_{fo}U_{yo}A_{fo}C_{yo}A_{fo}G_{yo}U_{fo}C_{yo}A_{fo}U_{yo}A_{fo}G_{yo}G_{fo}A_{ys}A_{fs}U_{y} | 21 |

In the table above, a “p.” represents a 5′-phosphate, a subscript “y” represents a 2′-OMe modified nucleoside, a subscript “f” represents a 2′-F modified nucleoside, a subscript “s” indicates a phosphorothioate internucleoside linkage, a subscript “o” indicates a phosphodiester internucleoside linkage, and a subscript “z” represents an internucleoside linkage of formula IX, which is a mesyl phosphoramidate linkage. Subscripts of nucleotides having an internucleoside linkage of formula IX are bold and underlined.

In vivo Tolerability and Activity Assay

For the in vivo activity and tolerability study in the table below, 4 C57/B6J male mice per group were administered siRNA by subcutaneous injection and sacrificed after 72 hours. The siRNA compounds were administered at 0.3, 1, or 3 mg/kg. One group of four C57/B6J mice was injected with PBS.

Liver tissue was collected, total RNA was isolated, and levels of HPRT1 in liver samples were measured by quantitative RTPCR with primer probe set RTS43125 (forward sequence: CTCCTCAGACCGCTTTTTGC, SEQ ID NO: 77; reverse sequence: TAACCTGGTTCATCATCGCTAATC, SEQ ID NO: 78; probe sequence: CCGTCATGCCGACCCGCAGT, SEQ ID NO: 79). Expression levels were normalized to total RNA as measured with RIBOGREEN®. ED₅₀ values were calculated by a least squares fit of data in GraphPad Prism using the equation “[Inhibitor]vs. response—Variable slope (four parameters)” and are presented in the table below. Plasma ALT was also measured and is presented in the table below. Elevations in ALT are associated with liver toxicity. The PBS treated mice have an ALT of 58.8 IU/L.

TABLE 64
In vivo activity and toxicity of siRNA to HPRT1

| Antisense | Sense | in vivo HPRT1 | ALT @ 3 |
|---|---|---|---|
| Strand | Strand | ED₅₀ liver (mg/kg) | mg/kg (IU/L) |
| 1337111 | 1448688 | 0.53 | 58.5 |
| 1405428 | 1448688 | >3 | 22.0 |
| 1465680 | 1448688 | 2.6 | 31.3 |
| 1465681 | 1448688 | 0.69 | 66.0 |

### Example 35: Design and Activity of Modified Oligonucleotides with Mesyl Phosphoramidate Internucleoside Linkages In Vivo to Mouse NOTCH3

### Design of Modified Oligonucleotides

Modified oligonucleotides comprising multiple mesyl phosphoramidate internucleoside linkages (Formula IX)and phosphorothioate internucleoside linkages were designed, synthesized and tested. The modified oligonucleotides are each 3-10-3 cEt gapmers with a sugar motif of: kkkddddddddddkkk (a 3-10-3 cEt motif), wherein “k” represents a cEt modified sugar moiety and “d” represents a β-D-2′-deoxyribosyl sugar moiety. Each of the modified oligonucleotides is 100% complementary to the complement of mouse NOTCH3, GENBANK Accession No. NC_000083.6 truncated from 32118001 to 32170000 (SEQ ID NO: 80). Each internucleoside linkage is either a phosphorothioate internucleoside linkage (“s”) or a mesyl phosphoramidate internucleoside linkage (“z”).

TABLE 65
Design of modified oligonucleotides with
mesyl phosphoramidate internucleoside linkages

| Compound | | SEQ ID |
|---|---|---|
| ID | Chemistry Notation (5′ to 3′) | NO. |
| 977131 | GₖₛGₖₛAₖₛA_{ds}T_{ds}A_{ds}T_{ds}T_{ds}G_{ds}G_{ds}T_{ds}T_{ds}^{m}C_{ds}AₖₛGₖₛTₖ | 81 |
| 1516016 | TₖₛG_{kz}T_{kz}^{m}C_{ds}G_{ds}A_{ds}A_{ds}G_{ds}^{m}C_{ds}T_{ds}^{m}C_{ds}A_{ds}A_{ds}^{m}C_{kz}^{m}Cₖₛ^{m}Cₖ | 82 |
| 1516017 | AₖₛT_{kz}^{m}C_{kz}T_{ds}A_{ds}T_{ds}T_{ds}G_{ds}T_{ds}^{m}C_{ds}A_{ds}^{m}C_{ds}T_{ds}T_{ds}T_{kz}GₖₛGₖ | 83 |
| 1516018 | GₖₛA_{kz}A_{kz}T_{ds}A_{ds}T_{ds}T_{ds}G_{ds}G_{ds}T_{ds}T_{ds}^{m}C_{ds}A_{ds}G_{kz}TₖₛAₖ | 84 |
| 1516019 | GₖₛG_{kz}A_{kz}A_{ds}T_{ds}A_{ds}T_{ds}T_{ds}G_{ds}G_{ds}T_{ds}T_{ds}^{m}C_{ds}A_{kz}GₖₛTₖ | 81 |
| 1516020 | TₖₛG_{kz}T_{kz}A_{ds}T_{ds}G_{ds}T_{ds}^{m}C_{ds}G_{ds}^{m}C_{ds}A_{ds}^{m}C_{ds}A_{ds}G_{kz}Gₖₛ^{m}Cₖ | 85 |
| 1516021 | Aₖₛ^{m}C_{kz}A_{kz}A_{ds}T_{ds}T_{ds}^{m}C_{ds}T_{ds}A_{ds}T_{ds}G_{ds}G_{ds}T_{ds}^{m}C_{kz}Tₖₛ^{m}Cₖ | 86 |

In the table above, a subscript “k” represents a cEt nucleoside, a subscript “d” represents a stereo-standard DNA nucleoside, a subscript “s” indicates a phosphorothioate internucleoside linkage, and a subscript “z” represents an internucleoside linkage of formula IX, which is a mesyl phosphoramidate linkage. Subscripts of nucleotides having an internucleoside linkage of formula IX are bold and underlined. A superscript “m” before a C represents a 5-methylcytosine.

### Activity Assay in CNS

Modified oligonucleotides described above were tested in wild-type C57BL6/J mice to assess the CNS activity of the oligonucleotides. Wild-type C57BL6/J mice each received a single ICV bolus of 300 μg of modified oligonucleotide listed in the table below. Each treatment group consisted of 4 mice. A group of 4 mice received PBS as a negative control.

Two weeks post treatment, mice were sacrificed. RNA was extracted from cortical brain tissue and spinal cord for quantitative real-time RTPCR analysis to measure the amount of NOTCH3 RNA using mouse primer probe set RTS36973 (forward sequence CATGGTCTTCCCCTATCACC, designated herein as SEQ ID NO: 87; reverse sequence TGTCAATCTCCAGCATCACC, designated herein as SEQ ID NO: 88; probe sequence ATCACCTCAGGACCCAGCTCAC, designated herein as SEQ ID NO: 89). Results are presented as percent mouse NOTCH3 RNA relative to PBS control, adjusted to mouse GAPDH RNA.

TABLE 66
In vivo CNS activity of modified oligonucleotides
complementary to mouse NOTCH3

| | Mouse NOTCH3 RNA | |
|---|---|---|
| Compound | Cortex | Spinal Cord |
| ID | (% control) | (% control) |
| PBS | 100.0 | 100.0 |
| 977131 | 30.5 | 9.5 |
| 1516016 | 14.5 | 8.0 |
| 1516017 | 21.0 | 11.5 |
| 1516018 | 12.0 | 8.0 |
| 1516019 | 10.7 | 6.3 |
| 1516020 | 48.0 | 4.0 |
| 1516021 | 4.0 | 2.7 |

### Example 36: Design and Activity of siRNA Having a 5′-Mesyl Phosphoramidate to HPRT1 In Vitro

siRNA Design

A double-stranded siRNA comprising modified oligonucleotides was synthesized and tested. The antisense strand has the chemical notation z.A_{ys}U_{fs}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} (SEQ ID NO: 90). The first 21 nucleosides of the antisense strand is 100% complementary to GenBank NM_000194.2 (SEQ ID NO: 22) from 446 to 466. The antisense strand has a 5′-mesyl phosphoramidate (z.). The sense strand is 1448688, described in Example 20 herein.

TABLE 67
Activity of siRNAs targeted to HPRT1 containing
mesyl phosphoramidate internucleoside linkages
and/or stereo-non-standard nucleosides

| Compound | Antisense | Sense | IC₅₀ |
|---|---|---|---|
| ID | Strand | Strand | (nM) |
| 1545957 | 1527118 | 1448688 | 0.02 |

### Example 38: Design of siRNA Having 5′-Mesyl Phosphoramidate Moieties

Double-stranded siRNA comprising modified oligonucleotides having 5′-mesyl phosphoramidate terminal groups (Formula XXII.) at the 5′-end of the siRNA antisense oligonucleotide were designed.

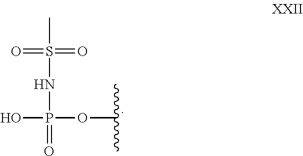 [SMILES: *OP(=O)(O)NS(C)(=O)=O]

Compound Nos. 1547257, 1547258, 1547259, and 1547296 contain a 2′-O-hexadecyl modified nucleoside (“16C2r”), shown below, wherein Bx is an independently selected heterocyclic base moiety:

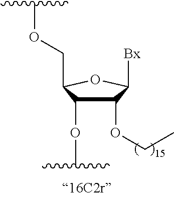

Compound Nos. 1547286, 1547287, and 1547288 contain the sugar surrogate glycol nucleic acid (GNA) with the chiral center in the S configuration (“Sgna”), shown below, wherein Bx is an independently selected heterocyclic base moiety:

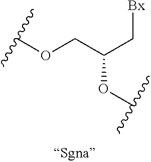

Compound No. 1448688 has a GalNAc conjugated at the 3′-oxygen of the oligonucleotide via a THA linker as shown below:

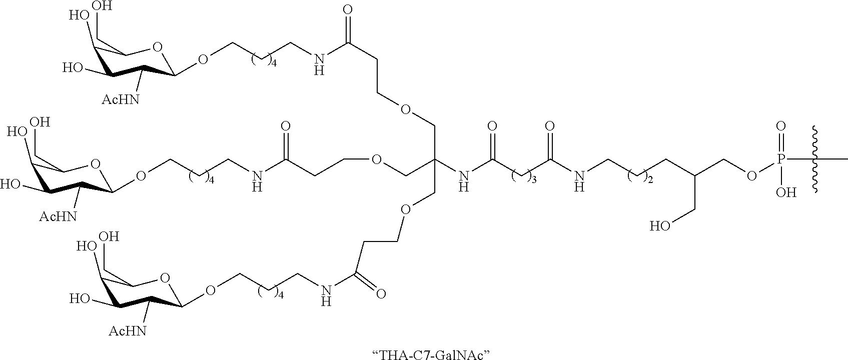

TABLE 68
Design of RNAi antisense modified oligonucleotides having
5′-mesyl phosphoramidate modifications

| | | SEQ |
|---|---|---|
| Compound | | ID |
| ID | Chemistry Notation (5′ to 3′) | NO. |
| 1547253 | z.A_{ys}U_{fs}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}A_{ys}U_{y} | 21 |
| 1527118 | z.A_{ys}U_{fs}A_{yo}A_{yo}A_{yo}A_{fo}U_{yo}C_{yo}U_{yo}A_{yo}C_{yo}A_{yo}G_{yo}U_{fo}C_{yo}A_{fo}U_{yo}A_{yo}G_{yo}G_{yo}A_{ys}A_{ys}U_{y} | 21 |
| 1547254 | z.A_{ys}U_{fs}A_{yo}A_{yo}A_{yo}A_{fo}U_{yo}C_{yo}U_{fo}A_{yo}C_{yo}A_{yo}G_{yo}U_{fo}C_{yo}A_{fo}U_{yo}A_{yo}G_{yo}G_{yo}A_{ys}A_{ys}U_{y} | 21 |
| 1547257 | z.A_{ys}U_{fs}A_{yo}A_{yo}A_{yo}A_{fo}U_{[16C2r]o}C_{fo}U_{fo}A_{yo}C_{yo}A_{yo}G_{yo}U_{fo}C_{yo}A_{fo}U_{yo}A_{yo}G_{yo}G_{yo}A_{ys}A_{ys}U_{y} | 21 |
| 1547258 | z.A_{ys}U_{fs}A_{yo}A_{yo}A_{yo}A_{fo}U_{[16C2r]o}C_{yo}U_{yo}A_{yo}C_{yo}A_{yo}G_{yo}U_{fo}C_{yo}A_{fo}U_{yo}A_{yo}G_{yo}G_{yo}A_{ys}A_{ys}U_{y} | 21 |
| 1547259 | z.A_{ys}U_{fs}A_{yo}A_{yo}A_{yo}A_{fo}U_{[16C2r]o}C_{yo}U_{fo}A_{yo}C_{yo}A_{yo}G_{yo}U_{fo}C_{yo}A_{fo}U_{yo}A_{yo}G_{yo}G_{yo}A_{ys}A_{ys}U_{y} | 21 |
| 1547286 | z.A_{ys}U_{fs}A_{yo}A_{yo}A_{yo}A_{[Sgna]o}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}A_{ys}U_{y} | 21 |
| 1547287 | z.A_{ys}U_{fs}A_{yo}A_{yo}A_{yo}A_{[Sgna]o}U_{yo}C_{yo}U_{yo}A_{yo}C_{yo}A_{yo}G_{yo}U_{fo}C_{yo}A_{fo}U_{yo}A_{yo}G_{yo}G_{yo}A_{ys}A_{ys}U_{y} | 21 |
| 1547288 | z.A_{ys}U_{fs}A_{yo}A_{yo}A_{yo}A_{[Sgna]o}U_{yo}r_{yo}U_{fo}A_{yo}C_{yo}A_{yo}G_{yo}U_{fo}C_{yo}A_{fo}U_{yo}A_{yo}G_{yo}G_{yo}A_{ys}A_{ys}U_{y} | 21 |

In the table above, a “z.” represents a 5′-mesyl phosphoramidate, a subscript “y” represents a 2′-OMe modified nucleoside, a subscript “f” represents a 2′-F modified nucleoside, a subscript “s” indicates a phosphorothioate internucleoside linkage, and a subscript “o” indicates a phosphodiester internucleoside linkage. A subscript “16C2r” represents a 2′-O-hexadecyl modified nucleoside, and a subscript “Sgna” represents a (S)-glycol nucleic acid.

TABLE 69
Design of RNAi sense modified oligonucleotides

| | | SEQ |
|---|---|---|
| Compound | | ID |
| ID | Chemistry Notation (5′ to 3′) | NO. |
| 1448688 | U_{ys}C_{ys}C_{yo}U_{yo}A_{yo}U_{yo}G_{fo}A_{yo}C_{fo}U_{fo}G_{fo}U_{yo}A_{yo}G_{yo}A_{yo}U_{yo}U_{yo}U_{yo}U_{yo}A_{yo}U_{y}- | 23 |
| | THA-C7-GalNAc | |
| 1505889 | U_{ys}C_{ys}C_{yo}U_{yo}A_{yo}U_{yo}G_{fo}A_{yo}C_{fo}U_{fo}G_{fo}U_{yo}A_{yo}G_{yo}A_{yo}U_{yo}U_{yo}U_{yo}U_{yo}U_{ys}A_{ys}U_{y} | 23 |
| 1547296 | U_{ys}C_{ys}U_{yo}A_{yo}U_{[6C2r]o}G_{fo}A_{yo}C_{fo}U_{fo}G_{fo}U_{yo}A_{yo}G_{yo}A_{yo}U_{yo}U_{yo}U_{yo}U_{yo}U_{ys}A_{ys}U_{y} | 23 |
| 1548003 | U_{ys}C_{ys}C_{yo}U_{yo}A_{yo}U_{y[XIX]}G_{fo}A_{yo}C_{fo}U_{fo}G_{fo}U_{yo}A_{yo}G_{yo}A_{yo}U_{yo}U_{yo}U_{yo}U_{ys}A_{ys}U_{y} | 23 |

In the table above, a subscript “y” represents a 2′-OMe modified nucleoside, a subscript “f” represents a 2′-F modified nucleoside, a subscript “s” indicates a phosphorothioate internucleoside linkage, and a subscript “o” indicates a phosphodiester internucleoside linkage. A subscript “16C2r” represents a 2′-O-hexadecyl modified nucleoside. A subscript “[XIX]” represents an internucleoside linkage of Formula XIX.

TABLE 70
Design of siRNA compounds

| siRNA Duplex | Antisense Strand | Sense Strand |
|---|---|---|
| Compound No. | Compound No. | Compound No. |
| 1547255 | 1547253 | 1448688 |
| 1545957 | 1527118 | 1448688 |
| 1547256 | 1547254 | 1448688 |
| 1547293 | 1547257 | 1505889 |
| 1547294 | 1547258 | 1505889 |
| 1547295 | 1547259 | 1505889 |
| 1547290 | 1547286 | 1448688 |
| 1547291 | 1547287 | 1448688 |
| 1547292 | 1547288 | 1448688 |
| 1547297 | 1547253 | 1547296 |
| 1547298 | 1527118 | 1547296 |
| 1547299 | 1547254 | 1547296 |
| 1548004 | 1547253 | 1548003 |
| 1548006 | 1527118 | 1548003 |
| 1548007 | 1547254 | 1548003 |

What is claimed:

1. An oligomeric compound comprising a modified oligonucleotide consisting of 12-22 linked nucleosides, wherein the modified oligonucleotide has a 5′-region, a central region, and a 3′-region, wherein:
the 5′-region consists of 1-6 linked nucleosides, each comprising a modified sugar moiety;
the 3′-region consists of 1-6 linked nucleosides, each comprising a modified sugar moiety;
the central region is a deoxy region;
and at least one internucleoside linkage of the central region is a mesyl phosphoramidate internucleoside linkage.

2. The oligomeric compound of claim 1, wherein each internucleoside linkage of the modified oligonucleotide is independently selected from a mesyl phosphoramidate, a phosphorothioate, and a phosphodiester.

3. The oligomeric compound of claim 1, wherein each nucleoside of the deoxy region is a stereo-standard DNA nucleoside.

4. The oligomeric compound of claim 1, wherein exactly one nucleoside of the deoxy region is a substituted stereo-standard nucleoside, and the remainder of the nucleosides of the deoxy region are stereo-standard DNA nucleosides.

5. The oligomeric compound of claim 4, wherein the substituted stereo-standard nucleoside comprises a stereo-standard 2′-OMe sugar moiety.

6. The oligomeric compound of claim 1, wherein each modified sugar moiety is independently selected from a bicyclic sugar moiety and a non-bicyclic sugar moiety.

7. The oligomeric compound of claim 6, wherein each bicyclic sugar moiety is independently selected from cEt, LNA, and ENA.

8. The oligomeric compound of claim 6, wherein each non-bicyclic sugar moiety is independently selected from 2′-OMe and 2′-MOE.

9. The oligomeric compound of claim 8, wherein each non-bicyclic sugar moiety is a stereo-standard sugar moiety.

10. The oligomeric compound of claim 1, wherein
the central region consists of 7-12 linked nucleosides and has the formula (from 5′ to 3′): (N_{d1})_{L1}(N_{d2})_{L2}(N_{d3})_{L3}(N_{d4})_{L4}[(N_{d})_{L5}]_{q};
wherein N_{d1}, N_{d2}, N_{d3}, N_{d4} are independently selected from among
a stereo-standard DNA nucleoside or a 2′-substituted nucleoside; with the proviso that no more than one of N_{d1}, N_{d2}, N_{d3}, or N_{d4} is a 2′-substituted nucleoside;
each N_{d} is a stereo-standard DNA nucleoside;
q is from 3-9;
wherein each of L₁, L₂, L₃, L₄, and each L₅ is an internucleoside linkage;
wherein at least one of L₁, L₂, L₃, L₄ is mesyl phosphoramidate internucleoside linkage.

11. The oligomeric compound of claim 10, wherein L₁ and L₂ are mesyl phosphoramidate internucleoside linkages.

12. The oligomeric compound of claim 10, wherein L₂ and L₃ are mesyl phosphoramidate internucleoside linkages.

13. The oligomeric compound of claim 10, wherein L₃ and L₄ are mesyl phosphoramidate internucleoside linkages.

14. The oligomeric compound of claim 10, wherein L₁, L₂, and L₃ are mesyl phosphoramidate internucleoside linkages.

15. The oligomeric compound of claim 10, wherein L₂, L₃, and L₄ are mesyl phosphoramidate internucleoside linkages.

16. The oligomeric compound of claim 10, wherein L₁, L₂, L₃, and L₄ are mesyl phosphoramidate internucleoside linkages.

17. The oligomeric compound of claim 10, wherein each L₅ is a phosphorothioate internucleoside linkage.

18. The oligomeric compound of claim 10, wherein each of L₁, L₂, L₃, L₄ are independently selected from a mesyl phosphoramidate and a phosphorothioate internucleoside linkage, and each L₅ is a phosphorothioate internucleoside linkage.

19. The oligomeric compound of claim 10, wherein N_{d2} is a stereo-standard 2′-OMe nucleoside.

20. The oligomeric compound of claim 1, wherein
the 5′-region consists of 3 linked nucleosides, each comprising a bicyclic sugar moiety;
the 3′ region consists of 3 linked nucleosides, each comprising bicyclic sugar moiety;
and the central region consists of 10 linked nucleosides.

21. The oligomeric compound of claim 20, wherein each bicyclic sugar moiety is a cEt sugar moiety.

22. The oligomeric compound of claim 1, wherein
the 5′-region consists of 5 linked nucleosides, each comprising a stereo-standard 2′-MOE sugar moiety;
the 3′ region consists of 5 linked nucleosides, each comprising a stereo-standard 2′-MOE sugar moiety; and the central region consists of 10 linked nucleosides.

23. The oligomeric compound of claim 1, wherein the modified oligonucleotide consists of 16-20, 16-18, 18-20, or 18-22 linked nucleosides.

24. The oligomeric compound of claim 1, wherein the modified oligonucleotide consists of 16 linked nucleosides.

25. The oligomeric compound of claim 1, wherein the modified oligonucleotide consists of 20 linked nucleosides.

26. The oligomeric compound of claim 1, wherein the oligomeric compound comprises a conjugate group.

27. The oligomeric compound of claim 26, wherein the conjugate group comprises a conjugate linker and a conjugate moiety.

28. The oligomeric compound of claim 27, wherein the conjugate moiety is selected from a cell-targeting moiety, a lipid, a carbohydrate, a C₁₂-C₂₀ alkyl, an antibody, an antibody fragment, or a peptide.

29. The oligomeric compound of claim 28, wherein the conjugate moiety comprises a GalNAc.

30. The oligomeric compound of claim 1, wherein each nucleobase of the modified oligonucleotide is independently selected from thymine, uracil, guanine, cytosine, 5-methylcytosine, and adenine.


SEQUENCE LISTING (93 sequences)

SEQ ID NO: 1 - DNA, 14836 bp, Mus musculus
  source 1..14836: mol_type = genomic DNA
cctcccccgtgtctccccacacccgggttggggttgttttggttgaccagagtggaacacaacgatctattggcagggctgaacaccaatgggtctatttgtaaagcgccaatgaccactttctgaagcagggttttagggagcggggccttagggaactctttggtcctttttagaacactggactttcttctggaaaggcaggaaacactgaagtttaagaagttgtttccagcttccattaactgaacacacattaaaaccaagcacagagaatcaggacgtttcgcgggagtgagacccagtcatttctcctccgtttccattctgcagggtgagagttgtaatcacccacccactattcgtaccatccacccacccccagtcgagagaataggggtacagaggggaggtggcaaagaaaattcacgatactgagtatctctgggagacctgtttggtctctttgctcggtagcgcagccctacgttagaatgcatcttcccgggaatgactgtagtgagactttggctgggaatccaagttattctaactgtagattggtccacgttgccctaagcctagcagtccactgcggcacagacaccctggacatgaggtgggtcagcttaagttcctggcacgaaagaaagggtactctggcaacttttggatgcggcgaaacagactgtttcgtctctcaggttcttatttcacggcttgtgcctttgacagccccttagtttctctatctgcaggatgggagcattaagctctacgacccagcctctttacaattcaggtccaaagagcccgcccaagttggggactgggaagatcaaaggtctcagcacccagcggagccgcggacactgagggcgccaagaagggggtgggtaggtagggaactggaagggcggctgctccgcaggggatgcgcgtcagagaccccagccacactccaggcccgccccttgatgagccccgccccgccccgcctggttttcgcctctaaagcgcccagcgctcgcctcccgctgccgcactttcactctcggtccacctcggtgtcctcttgctgtccagctctgcagcctccggcgcgccctcccgcccacgccatggacgccaaggtcgtcgccgtgctggccctggtgctggccgcgctctgcatcagtgacggtgagtgcaatccgcggccgggcccgggaaaggctcgcagctctgcgccggagctccttcgggtccgcggttcctctgcccgcgccgaagtcgcggagaaagaactcggtcggcgccgttcactacaagcgaacttggggcagtccactttgcagggcgcactcccaccgggtgccctttcccgtgtcccacgggtcgcaccgaggttttgtgctctgcgaagtgcggccataggacctagagagggctgcaggggaggacccgcaggattgttgggcaagagtgggttcggcgcggaatggaagcgtgggcgattgtgtccggggcttgggccccggagcgcgccagctgcactcagctagtgtctaccggcgcccagatgtttccagaggcgaagggcagcgcggtcccggagttgaccgtgcaagaggttcactcgggtggtgcgtgtgtcagcaaactctcaaagaccggtcaagtagctcgaagtgcatggcttggctataggttcagtggtgaggctgagtttcgtcccctgcgggtgtagcgtgttctcttacagcaccctcgaggggctcagggccaccagcagcgcagcgcagctcttgaactcgcgctgccagccagggccgcgcttctgcacagttcgttggtccgtagcgacgcggacctgagcacgcgtctcttcactgcccctttttcttcttacccgggtcactagacaaaggctcagcagttacccaagctatatgcacacctctccccaacccccaaacacacctgcaaacgggcgcttttgtagccagccccggagtcctcagctctggaatgagagctgcagcggagttcagtctcccagacccagggtggtgtcttctttcactgggaaagggctttcattttgttttctttttttgacactgaagagaaaactctcagcgctgttacaagacaccgttgctgcaaaacaaaacaaaccattgcctctgaacacaaaacaaaatcctactagtcgatcccctgccttcctccgcagtggtgtttcctggagagaactgagggacagtcggggctcttggtgagactgagctctaaatgctgcccaagtacaccaactcgttcgtttgggttctttccctgtgacaacggggtacgggaatggttggagttgcctagtccgagggaaatgttctgtaaaagaatagtcagttgctgatcggagtagtaaaaaaaaagaaatgaaaggcagtttcgatttttttttttttttttttttttttttttgttaccgagaacacccgggaggctgagccttcccactggtcccccagtgccccgtcatggagcacattgatttgggcattaataattgaatgagctggtgatgttgcaagggtcacagcctctggcaagttaggtatggggcaagaatgtaggactcaggtcctcaaggttggagtgcaattatccagagtaaaagttgtctcaccctcaacatattctgaccctaggaagagtcggattgttgacagtgtctggatcagacctgttctctaggcaggaccccattgtgctgcccgaatgaacttttttacctcctagtgcctgtgtgccctctgatcttacacagccctcaagttgcagcacggctaaccttgctgtggttcctgtcttttcccatcagctactccaactcagaagctagatagtagacaccggaggcttctttggttaaacccagagcagcaggcttgccaggcttgttagattgaatggacccctggttccctaagccaagctctctagattcccaagtccagggtggcagcagagctggattagactttggtctgtacctgaagtctggttttcctatgctttagagtctaaagacactacccttcctggggcatgcatcccttagctaaataatgcttgcagaagaagataatcccatcatatatttaattcggtccacttctccagctgcttcccaaaggcagtgaacttcagaatacccagaagtctcctggaactctaaataagcaaacttaaaatcctggggctaactattctcagtcatacttttaaactttggtgaaaagacccataaattgaaacatttggggatgctcagtagagctaggataaaaccctgttgttgggggagcagctacaaatccagcagtcctcagggtttgcaattctagacttaaagggtggttcttaagggggggttctaaaggagccccttgctaatttacactaatgagtgtcaattatagcattttgcaaattggtgaattggcaaacaaagctggtaataggatccaggaggcctaggcatccaggtagtgaccataaaagccacggttgaccccagcttttgggaaaagctggatagaaggtaaatccgggtcctcccctctggattcttttgtgatttccagggcttaggatagggtgagtgggaggagggaaaactgcaggtggtagaagtgaagccccccacctccaggcctgcaccagagggccacaagggagcccagaactctgccaccccacttctcctgggtccttttgtcctttagaggctgagcccagtcagatctcactgtgatccctggccgaggggatggtctttgcaagaaactttctgtaaccattcctgctgatgttcctgagtcttccccacaagagccaccaaaccccctgcaccaggcagataatgactggccccacttttctctctacacctcctctaggtaaaccagtcagcctgagctaccgatgcccctgccggttcttcgagagccacatcgccagagccaacgtcaagcatctgaaaatcctcaacactccaaactgtgcccttcagattgtgtaagtcctagccgccatcccccaaagaggagcatggtatagaagcctcggacttggcataactaggggcagctgttaccaccaccaccacggggacactgatatgccatcagacatgggtttcaaaggatacttttgttccccagagccctgatgtcctcagtgtttctcactcttgctttccaagctgtttcttgcagcacagtgggccgcctctctacagaaaaagccatggacttgatggaggtcagccctcagctgacagttgggtctgtcttgtcagtttcaaggttctggtgtccaaagttaatcctttctcacatagaaaaaaaaattacaagacccggatggcacggggggggggggggttcagttttactcacttgcactcacttgctcagaggtcatttttgttttagagttttagagtttgctggagtgtgatggtagctgccagtatttgatttaaatttacctgggaaataagaaaagcccaaaaaaggtataaatgatgtgaatatctcactcagagtctggtagacttggcagagatgtgtcctgtgctagtctgtcctgctcactgccccccagcaggggttcccatcctcgggagactcaacactaacaacagtataaggatgcagcagctggagcaatgctagcctgacggctttgtcacccaacggtgactgcttcagactttctgtgctcatcagccttcctctccagcctccgctgctgtgttatgtacagtaggctttagagacctagatgatgaatattatttttgctgttttgattaaaatacaatactctcccgagaaagggattttaaagatgatgagtttacgtttgaataggctgtgctggtgcactgtcccgggaagggcccttgaacttagagggtcaaatacaactattgattctgggtgatcactaagttaataaatggcaggatccagactgacacccctgatccctgttgaagttacatccctctgaacgactggtcaactgcagggcagcctgcttgaagagggttacctgtccctaggacactgaacaggcatttgtttttcctagaagacagttcaccagctggagaggagtcgtctcccgtagtttctgtttggttgcttttggtttttgtttggttttggttttttaattatctggcatccaggacttgatggaaaataaccagagctaagctcaccggttcatctgcccattaggaagttctagggatgggagaaagaacacggcgtcaattaacaaatccacaaagctaagaccttgaagcattctgtgaacttgtaaacgcgctcaggcaaccattggacaatttgtctagactgctccttgcccacctgaactgccctgttcctccccttctggactcctgccgtcttcctccagagctacctttaaggttgtcccatgtactatcaaggtgctctgtcaaaagttcttaggctgcttctggcactctccagaattttccaagacctcccccccaccatgatatcagtcatccgcgccttctgggtggttcttcctccacaccctttgggcactttgactcctgtgggatattcgtccttccttttcctttagctttcctcacttgccaagctccaacttggccagaagctcaaatgcctccactgtggtctcttctctgtgtcccctgggagacatccttagcacgtccctaactctgcggtggtggtcccaacacgattcaagtgctatgtcttccaaaactgaagcttccgggagcagcagctgggccctgcagtgaggacctttagctgggtgtgttgggtgagcccacaggatcgctttctcccgcttggctgtacagcgtctctccccttgtgttttggcagtgcacggctgaagaacaacaacagacaagtgtgcattgacccgaaattaaagtggatccaagagtacctggagaaagctttaaacaagtaagcacaacagcccaaaggactttccagtagacccccgaggaaggctgacatccgtgggagatgcaagggcagtggtggggaggagggcctgaaccctggccaggatggccggcgggacagcactgactggggtcatgctaaggtttgccagcataaagacactccgccatagcatatggtacgatattgcagcttatattcatccctgccctcgcccgtgcacaatggagcttttataactggggtttttctaaggaattgtattaccctaaccagttagcttcatccccattctcctcatcctcatcttcattttaaaaagcagtgattacttcaagggctgtattcagtttgctttggagcttctctttgccctggggcctctgggcacagttatagacggtggctttgcagggagccctagagagaaaccttccaccagagcagagtccgaggaacgctgcagggcttgtcctgcagggggcgctcctcgacagatgccttgtcctgagtcaacacaagatccggcagagggaggctcctttatccagttcagtgccagggtcgggaagcttcctttagaagtgatccctgaagctgtgctcagagaccctttcctagccgttcctgctctctgcttgcctccaaacgcatgcttcatctgacttccgcttctcacctctgtagcctgacggaccaatgctgcaatggaagggaggagagtgatgtggggtgccccctccctctcttccctttgctttcctctcacttgggccctttgtgagatttttctttggcctcctgtagaatggagccagaccatcctggataatgtgagaacatgcctagatttacccacaaaacacaagtctgagaattaatcataaacggaagtttaaatgaggatttggactttggtaattgtccctgagtcctatatatttcaacagtggctctatgggctctgatcgaatatcagtgatgaaaataataataataataataataacgaataagccagaatcttgccatgaagccacagtggggattctgggttccaatcagaaatggagacaagataaaacttgcatacattcttatgatcacagacggccctggtggtttttggtaactatttacaaggcatttttttacatatatttttgtgcactttttatgtttctttggaagacaaatgtatttcagaatatatttgtagtcaattcatatatttgaagtggagccatagtaatgccagtagatatctctatgatcttgagctactggcaacttgtaaagaaatatatatgacatataaatgtattgtagctttccggtgtcagccacggtgtatttttccacttggaatgaaattgtatcaactgtgacattatatgcactagcaataaaatgctaattgtttcatgctgtaaacctcctaccgtatgtgggaatttatttacctgaaataaaatctactagttgttagatggagtgcacatacatttctgaagatggagaaaaacaggtgtgcctgctgatcaggtgctgtgggctgccctgcagtcctggtgagcgacagacactgaggcaggcttgtctcatgaacaggctgcctctgcagtgaaagtttttgtgtattttttttaacccaagctagttttctaatgaataatacttgactcactaatttcccctcctcctccttctcctcagttctcctaacatcctcatgtgatccccagactcaactccagtaatatcaagctttcctattttcccatgtaaaaaaatcccatgactctgggccatgttaatatcaggcttttgtgggaacaggtggcctcaccccataaatcattaaataccattcagcttgaatcattttaatgtgacagtcacaaaccagttgctctaataaaaactctgctaaccatccttctccttagctctctagaacaatctcagttatccctagggatgctccccagcatccagaaaagagaagtgggatcaatcatcctgcctttctccccctcctctcttggagggctgcctgagcccgtggcctccacctcccctgctttgtataatttgaaatgcagatttgtagtgaaggcagagttcacctctgcattgaaagggaaggcaggcccagagcttccttccctgccctctgagatgtgcatttatgtctcaggatggatgagctttggtaggaatgctcaaaaccaggaccagccagacaaactggcagtccctgtaagcggttcccgggtcatagggttagggcacccctgtttaactttggggtggggaaagtatctggttttctttgataaattgcttgtgaaccacatttgccaagtggcctccaggcctcaaactcaaagaccgagctaaatcgactcggaaggcaatgctgaatgaagattgtgggaactgagatagatacactcctctatgttgcaatgtgattaatggttctactaattttatctaagggggcgcagagaagaaaaagtggggaaaaaagaaaagataggaaaaaagaagcgacagaagaagagaaaggctgcccagaaaaggaaaaactagttccccgcttcctgccgatggaccgcagtgcgctctgctctggcgctttgtaactcgctcctccctcttcgggggcagaccccacactccgggcaggtgctcaaacctgacggtaaactcttccctcttcgggggcagaccccataccccggggcgggtgcttaggctttcctgccctggtggccacaccagctgctgtatttatgtgcttcataaggccctgctctgtctgctaaagctatgaagaaagatgtgcagagactggggtggagactaagccaaagaggagctgcctagcctggcagcattgccccgagctgagcccccttggccaggacttcacaaggctcacacctacaatcccatgaaggccagggtggtctgcttagccaggaaagggcaagtgccttcccctcggccacactgccccttgtggccttctcgggacatgtggtaactgacttgctctcaggcccacccgcagcttttccaaatacctgcagccttcagccctgctgccctgcctgtgggagcagctttgactccagtccagaagggtttctgcagactgtgttgggtgagacgcagaaaggatgaaatctcagaacacatgtcagctgcttctcaggaaatcttttctttggacaattcactttagagtctttaaacgggtctctcgtggggaggatagatgtgctctggaactttctgaaggaccagcagcttcagggactcttagtctgtccttccccacttttggtcccaacatccctgggatggtgtgctgtctgggcaccacggtctccatcctcactcctgagagatttctgccttctgtgagttgggttaaagctctggaattatctactatcccaatccactaccctcacctggcaatatttgtctgtttttgtttgtttgtttgtttgtttttgtcttttgccagtttgaattagaaggcaaggctctgattttagtagtgttttggaaaaggacttttttcttcaccttcctctttgcctcatgtgtacacacacacacacatcttgtaccccagacctctgggtataattttcataattggtgcagaaagaagaaatgatctgaagatgtgttaaatggattgcaggggaaggaaggcccagggccctgtgtgtcatgccctcttgggttcctaagttctatgttccttagaggttctagcattaaacagataaagcccttcatggtcctggctgaggaagagtcttgctagggggattcagggaagacccgtgttaccagctcttaccctttatctggacagctctcctaccctgtatcttctcctcagatctgaggatagcaggctggactattggtgggcacctttcaagcccagggctactgtttgtcctgtggcagccggctacagtctcgtctgagtggcctcatctggacccttcctgttattaataaaacgcttctggaggccagatctgtgctcaagccatagttctgcttagaaagggatgccccacccttaccggacactgggaagaactgttggcccctagaaaccaaaggccaaactgaggctgccctgagttggaagaccactttctgaaatgcccatggactctgcctcccaaccattcgtctctcactcctagcagagctgtctgtgcagactgtttcttaggaggcacagcaagctccagggaaccctctgtgcttatgaagctcgtctggtgggcaaccccagcccactggacagagtcctcatggaaatgcctgggaagctgatttcatctaaggatgggttgaagtaggatgtgctcctgcgacttctcaggcaggtgagaggggtagtccttacactgtctagcataaacgccttccggaaggacctgcagctccagagaccacctcctgagcaccaagacctcttctggtggtgtggaaccagccaagagatttcaaggaagagtgattatttgatgaatgctatgggaatggcctcttctcttggagttctgaggcctggggatgcccaggaacactgggcacctgctgctgttagggccaatgcatagtctcagcaccggtgtcctaaggttaaggcggtgcgccttgtcatgtgctccttgtaccatgccatctgtgccagtgtgtgtctgcctcaccctgtgcttgacatgttcacccatcttctctgcttcccgccaccatccagatcctcagcggccgccccggctgtgcccttccctgctctcccgctctctcaggcctcggaaggaagatcggtggctgcgagctgaactaaggagtagggcctgtggctcagcgctaggccacgcacgcagcatcccaggcatgtggtgagaaactgccttaatgtgtctcctctgttcttgtcaacaggaggctcaagatgtgagaggtgtgagtcagacgcccgaggaacttacaggaggagcctaggtctgaagtcagtgttagggaagggcccatagccacttcctctgctcctgagcagggctgaagccgtttgcaagggacttgctttgcacagttttgctgtactttcacattttattatgtagcaagatacatggtgatttttttttttttcatttagcctgattttccaacgtcattggtgacaggccaaggccactatgttatttcctttgttctggtatccttcccttggaggaccttctctgagtagtggctccccaggtttgtcctttgagctgaggcaggaggctcacccattcttctgaataggaactgggtgttcccaccccccaaggactgcagggctttcccaagctgaggcaggaacgtgaggccagggaagagtgagcttcaccctcatcccacgctgtcctcctcaacccaccatgctcatcattctgtctcatccatccatccatccatccattcatcgccatgtgtccgcaagactgtctccatgaccctgaaaaaggactctcgagatgaaatcctttattcaaatgggacagcaagaaggaaaagccaatgtctggtgtctctccccccgcccctaccctgcgcgcatctatgtcttgtttggaatattgtctcttcaaccccctgttcatgtccttctcactcatgatcgatgtcttgtctgtgcactgtctctaacccaaatgcaaaggctgagtgtgaggtgatggccccgaggtccaggttgtagtcatggaaagagccctgctgtctcccttctcagggggcccattttagacacacaaagcccaaagaaaggtggtttgcaacagtgcttagctcgagcctccatatttccataactgttagcttaaaactgtggggttttaccttcctggaaccaaatgcattcttctgttgaggagtaacaggtctcaattcttttcaattaattttaaaagtcaatcactaagagcatcggctttgggccctgatgggcaggcatttccctggaaagggggtgaactacctacctctcctcaagacagccgaagggtgggattggtgccgctctgggaagcgtggccccaggagttttgtcctctgcagtttttaatgcaagttcactgccactttgacaaaagcccaattagaagccagtctctagttccttaaacaaaacagacagagtaaggaaaggaaggagggtggcagccagctggctggacactcgagaaagacggggaagtaagctacagaaagatagtcttcaaaaacaggtgtttgagagtgaatactctgtagaattgttagtggggtgtgtgtggtggtggtggggggatttctacaaaatagtcctttaagttgagtttacagcagatgaaaaatccaaccagcaaaattttgatcaaatttgaacaaaaacccaaaaacctaaaactgttgagcaggttgcgatgaggagcacagggctagctgcagagctggatcctcaggaggatagcgaattattttcaaccctggaatagaaaccacacactggcttgctgtgcaccagccactttgcatctaatccaagctttgaagggtgttgcttgggaggaaacaaatacagccttccatcttcactccagttagggatcctttcaaagtctccttcacagtgaggaaaaagagaagggtagaaactttagggagccggatttgtgtatcaattcctccgctgacagtcagtttctagatggagacagcctgcttaaagcaaatccgaatttaaataggacatttacatcggaaaagtctctccctaccttaatcccccattctcttgctttcaaaatacaagcacagcagtccttgaatggctgttgacccagggcacctagctgtccctgctggtcctggggctgccagaattcccttgggcgccaagcaacctgccaggtagccagtccctctgttacaagcctttgcatctggatagggaaaggggtggagacatacagtctgctttgtgttgaaacccagatttgtaccctgtgtttatacactgctgctggctcccgaggacagtgggactttagcaaggaagtgcagccgaggggtaaagagccctctggttcattgcctgatcggctttgagagagggtttggagggcaaggggctgcattcctctgagggacttggcctgaggcctttcgggcctctccagtgggttctgtttatcctctcatgggtgattatctcagtggtgtcaccaggggcttcctcccagaagtcagtcatccccaggccgtgcacccttttcagctggatgagagccagggatgcattctctccaaacagctaccctggcccattttaaggtaatctcattcttcaaaatgttccatagaatcctccaaattcccccagcagacttctaccctcgccaagttcccaaaacccactcagcaaagttgccaacctcgacgggctagcagtgtctaagcagcgatgggttcagtgttgtgtgtggtgaatactgtattttgtttcagttctgtctcccagataatgtgaaaacggtccaggagaaggcagcttcctatatgcagcgtgtgctttcttattcttatttttaatatatgacagttatttgagaagccatttctactttgaagtcattatcgatgaaagtgatgtatcttcacctaccattttcctaataaagttctgtattcaaatatagctgccaagcatcctcagtgaatgttaccatgtggaattttccacacttggttttaccccctcaaacctgactctgaccgtgcagtcttagcagaagagcttagcaggtcctagtgttcactcttggtctaactgctggtgtcagaagatctctacagggagaggtgttccattttctccacatgacctggattgctccttagaggtcagacagccttgcactgtacaaggcaatggcttagggtaaagtcccaggagttttccctacagtcccaagaatttggaagaggaaggcccacactacacatgcaggtcatggtggaaggtgacagaggaaggactctgtccctgtaagacagctggaaaccacaatattctgcatgttcctatcctgggtgaggacgctaatggaagtcaaaggggaatttgctaactgctgttggccagcttcctccaagaatcctgcttccccaacagacagagcctttgtctcttatagtttggtcttcagattctctttatcccacattcagccatttttgtaaaagagaggctagcaccagctccaaatatccaaatctgcagtgtttgagatctcactgcgcctcctccataccaacacatttgccattacttatagggtagttttcatgtgagttctaagttgattaacacacaagaattagaagggtgggaggctctaggaaaggcactgtgggactatttgactgcatgggtgtgaaaatgtaaggaacaggcaagagcttggatcccattctctctgcccacattgtgacttgagatatactaattgctcttgggggtctcagtcatataccatccataacagagttaaactgagagagatacaggatcagctagaatgaaaagcccaccccatgcttccagaaagtcccctctttatacctcctgtgatatgaactagaggaaaagcaattgactttgcttctcaaacagcctacggcaaagccctgtgagtttg

SEQ ID NO: 2 - DNA, 20 bp, synthetic construct
  source 1..20: mol_type = other DNA
ccagagccaacgtcaagcat

SEQ ID NO: 3 - DNA, 20 bp, synthetic construct
  source 1..20: mol_type = other DNA
cagccgtgcaacaatctgaa

SEQ ID NO: 4 - DNA, 29 bp, synthetic construct
  source 1..29: mol_type = other DNA
tgaaaatcctcaacactccaaactgtgcc

SEQ ID NO: 5 - DNA, 16 bp, synthetic construct
  source 1..16: mol_type = other DNA
gcatgttctcacatta

SEQ ID NO: 6 - RNA, 20 nt, synthetic construct
  source 1..20: mol_type = other RNA
gataatgtgagaacatgcct

SEQ ID NO: 7 - DNA, 9001 bp, Mus musculus
  source 1..9001: mol_type = genomic DNA
ggcagatttctaagtttgaggccagcctggtctacagagtgagttccaggacagtcagggctacacagagaaaccctgtctcgaaaaaaaaaaaaaaatccctttctctggctataaggatggctcagtggttaagagcactgactgctctgccagaggtcctgagttcaagtcccagcaaccatctgtaatgggaaataatgcactcttctagtgtgtgtctgaagacagctgtagtgtactcatataaataaaaaataaatcctaaaaaaaaaaagaattcccttcctcttattgataccctttcttgtctctagggaccccccatatatctttctagacatttctgagaactcatgtaaatacatgctgagccccctctttgtagtttgtaacctttgctcattccataccattttaacaaatattttccttgaaacactatttctcacccattgcatggaggtatcacataggactttatcaggcatcctgttctccagtgtgtggcttagagccagtggaatgcacggcgtgtccgagaaccacttcacacagggaagagaatacagatttttactcagcaagtaacaccagctgggaatggtggggcagacaagcaatcctagttactagggatgctgaggcaggaggatcttaaattcaagtttagtccctatctcaaaaattaaaaaagaacccctctaaccccagcaactgagaggcagaggccagggagatctctgtgagttcaaggtcagcctgtctgttctacacaatgagttccatgagagccaaaggtacacagtgtgatatttttaaaaaggtatgtgtgtctttttttttttcaaatttttattattttcttcatttacatttcaaatgctatctggggagtcccctataccacgcccccctgctcccctccccacccactcccacttcttggccctggcattccactgtactgaggcatataaatttgcaagaccaagggcctctcctcccagtgattgtcgagttggccatcttctgctacatatgcagctagaaacacgagctccggaggtactgggtagttcatattgttgttccacctatagggttgtagaccccttcagctccttgggtactttctctagctcctccattaggggccctgtgttccatccaatagatgactgtgaacatccacttctgtatttgccaggcactggcaaagccttacacgagacagctatatcagggtcctttcagcaaaatcttgctggcatacgcaatagtgtctgcatttggtggctgattatggcacggatctccaggtggggcagtctctggatggtccatccttttgtctcagctccaaactgtgtctctgtaactccgtccatgggtattttgttcgttattttaaggaggaatgaagtatccacacgttggtcttccttcctcttgattttcttgtgttttgcgtgtgtgtgtgtgtgtgtgtgtgtgtgtgtgtgtcttaactggcagagcacttgtctgtcatgcagggggcggggtggggggtggggactgtctaatctccagctctagtaacaaaaataaaagaagtaaaaaataagtaagaaacgggggtgtgtctagagatagaacatgggctttacacattttagacatcatgagaaaataaagctggaaatgacactgggcatccatcttggcgcatctcaactttcacactgcaaccgaggcgcgctgtgcaaagtcagtgacaatccgcatttccagacacagtgggttcagaccttccaggcgcgcacgcgggcctcgtgttctcggtttccgcggcgactcggccgacgtcacagttagaagacaatagcgactttccccgctcaggctcctcgggaactttctcagtccgcacgctccaggagccggagctaccctccgccccgcccccagcgtgccccgcggccagggagctccacgaagggcgggcggaggccgcgggtagcgattggttccgtgccaaggtgggcgtggtcagactcaggcctataaaagctccgtggcgccagggcctcgtttttttgcgcggtcctttcctgcggcgccttccgtccgtcggcttctcgtcttgctctctctggtccctccggaggaggccgccgcgcgtctcccggggaagcatggcgatgaaagcggtgtgcgtgctgaagggcgacggtccggtgcagggaaccatccacttcgagcagaaggcaaggcccggggcgcggggcgcaggccgcggtgacgcggcgcacctgtgcgggagcacgccacgcccccgccacggcctgagcccgctaagtgctgagtcaccgtggcctggggcaggggctgggcgccgggaagcgaggcccggggcgccgcggggccttccgggcgggcgggggcctccccgcgccccggagcggctgggcctgcccgggagagccggcttggcatccgttatccttctggggctgctgcttttccggtgtccgtgtcccacaggctcagagccccgtggccaccggctgcgattgttgtaagaatatttgaacccggtggtgccagaccggactaaggccgcaggacgcgtttgcggcactttaaagcaaagtcctgggctgttctgtactaggtcagggtcgtgtcgcaaggcggaaagaaagagatggccttggacagccgtcccttgctttgcactccagagagagacccggctgtgggttttttctaccacagcgagtttctgagcacattttggaaaagtacatagagatattttcgaaaatactgtgaccctgcaaaaacacatgcgtcacagggaagatgtgtgtggtaaggttgtgtccagagccttagggaggttaccgttgttgtattcaccttaatcccgagagaatatttgataaatgagcgttatgtgctctctgaagtggtggacatacgtgtgagaaggcagacaccatagtgaatcccaagtgtttggtttacgacgagaactgataacggcaatttagagtttttcgtaactagcctcgtttccagcagtttcttggcattgaaatgcgttttgttgttttcctgtggaagttttttgtttttgtctttttctcctccccacgtaattcactgtgagaaagacgaagttcggctgggtcttacccctgtgtgtgggtttctgtcatcttccaccatgccatgccagagcagctcgcactatttttgtgacgctgcaaactacacatcgctggtgccctttgtacccaatgaaacgatagttaagcattccagattggcagttgtaatcaaagctggttgatttaacctgttgccaacttttcagaatcagatttttctacccaaagttcatattcccttattctgttgcaaaagttggacatttaaaaaaaaaaaaacctaaaaaatgattgtccttgcttgttggtcggttgctcttacattttctccctattgctacactttctggagcagtactaatttgaattttgggtgttcttttcttttttgttaagtggcaaattttctagatttggatagctaatgagattttttttttaaggtagctctggttagacccaaatggatctccacaggcagtaggacaaaggcattttctgaccactaaataaaaataggggaactgataaaatcactgaatgtggagaacagggttctcggcagccagtgttctgtaagagtcaagtctgacagtgcagtagccatctcttccccaggcctggcattcagtagcccctgtttgttccacctggtgctttctaaatgctgttcagtccaggtgcctgcacacatggcatctggcagcaagtgttaggagaagtgtgacagggagagagaggcctagagctgagcgtctccagagccaccctgtaggaagtgggtctacttggatctgaacataggtttgattttcactgttgtgtgttttgacttgagctttttactgtgcttggttagggtgtaacccagcaacagccctggtgcaggagtatttacactcaaacttgatgtcttcatttttgtatttttttaaatcaaggcaagcggtgaaccagttgtgttgtcaggacaaattacaggattaactgaaggccagcatgggttccacgtccatcagtatggggacaatacacaaggtaggtcctaggctggctagtgaccagtgatggaaaggaactgagtcaggacccaattactaaccatttaaaactatctcgtttgttttctttttcttttagataaagttaaaatgaccacttaggtcaaccttggaaagtagccacaaaagtattttatttagtatcaagtattgcttgcttccttaagtgtgggaaggtaaagaaggtgatttttcttcattgtaattataattaagcagcaccttgcttattctgggtgtttattgggtgcttatttgggtgtttggagctgggcgttgaggatggatgcattaggcagagtgtctaaggacaaccatgccttagcatgagaggcatagcgggacagaagtgacaaaaactgaagattcaatataaatgcttaagtaagatttattttctctatttgggattagaatcaagtcagtaaaaagtagtggcttaaattgcagttagtgaacttttaccatattggagtaatgatctgaatttgcttaccgtcatttaagagcctcatccatgttgcgagagccttttcctttcctctccttctcccctgccttcttcccttctccacatagcccacggtggcctggaatttagtcttgtgtgtgctgtgttaaaggcatgtaccatcaacctgtgtgctatgtgccataatttgttctacagttacttaggattgggtttgacccatttgataattactaaagttaccccgagttgcctctggcctggtagctttgatttgttaagctccttccagaatcctgcccagttcctattttcttggtctgagtaaacactggaagtcctgcatataaaaggacttgctgcattgttgagctgtgccttgtgactggcatcccttagcccacatgagtagtgtggtacacctcctggagttgaggacaccagccctggcccttgggaacaagccatctaacagtctgcctgccccaagtaaaagctagacaggtgagctgtttggtggcacatggtctagaaagataagtatttttatcatgaagtatgctcccttcttaaaagccaaggtctttaaatgtgggactttaactttagaagtgccattaaagatcacatctgttccagttacaaggaaggaacaagagccaggcatgctgtcctgacactgccatggccctaaggctaaggtgggagggtcataggtcgcagatatcctgagctgtagtagtgagacactgtctcaaaactcaaaagcaaacaaaaagaaaaatgtgacagtctaggaaaaaaaggtagcctgagaatgtaaggctatacagtgcagctacttacaccagggcgctgctgcctgtttttatcgccccagcacataccaggtcagtgtttgctatgttggaggttgtaagaatgcctgtgttgttacatataggggtttacttcataatctgactgctggtttctggtaaataggctgtaccagtgcaggacctcattttaatcctcactctaagaaacatggtggcccggcggatgaagagaggtgagcagcacgctctgtatgcatggtggaggagaggggtctgtggaggaccccagtaagacagaactgcatggcctcctgcctctgcttttgtgtttgtttccattcacccaactcactcccacaaccccacgtgctagaatagcttctgttgggtgaaggagctgacaaatgtggactcttaaagtgatttggttttgtagcatttattgaagatgaactaatacaagtgccaaaaggaaccaatacagaaaatatcatggataacagtactgtcagtcactggcaaagtaaatcattgtataataggacgctaatgcagataatgaaaactagttgagattccatttgtatgtgaaaccttaggaaagtcctaaataaagaagggctagcctgtttttagaatgggggcctgggagcaaacctttgctaactcaggagctggcatactttactaaagccccagattatgactcttctcagagcactacctttaaacttgaagaactgtctgtcaaggtatcctgtagctacctgttttgaactttgtgtttccagacctttgccggtctggaaaagccatcatagttgataatgtatgtgtactttttcatccactcatacgtatttgacttagtcagattttaacttagttattgaactctagtgatgtgaaatagacatcattgttcatccacctgatgctgttttaatgttaggcatgttggagacctgggcaatgtgactgctggaaaggacggtgtggccaatgtgtccattgaagatcgtgtgatctcactctcaggagagcattccatcattggccgtacaatggtggtaagtttttcatataaggatatatacataggatttcttctaacatagttatgtaccttcccatgactttatggtggttaaactagtttctaaagagtcacataaattgttaagagttcagggtaggaaaaaagttcttttattggctgtgatagtaaagaattaatttgcctaggtcagttaagaacactgttgtgctgaaatgcagtagaaagcagttacatttgatgagactggatctgagttgaggatacaatagtctttagtctaaaacagccggattttcttgccatgattgcccccccccttgcaacatttcgttgagtctaaaatctgcgatggatggcagtattcaagtctgtaggttatcgcttggttaccatatgggagccgtcttcccaagttaccctcgggagatgcatctgggtcatgcagaacaccaagtagtaaaggctcttgcccacctcgggcagctaacttttcagtaggcacttccttccttgcagttgaccctttatccttagaatgctcttcagccctattggtgaagcagaacagtcattcataagtgttgtaaaataaagctttagagtcttgttgctaagtagagatacttagaattgcctcttatgtgtaggcctatagttctttcaacatgagattttgatagagaaatttgtaagaatgactactgtgtagttggggaggagctaagatcagcatgtacctggtagttacttgggtcttagtatttcatctagaaatagccactagcaaggaaaaacttagtggtctgctcttaactgctagtatttaagtctgtagtattgctgggaagaagtactagttacttgatcattcaaacctaaatgttcttcttttcaaaggtccatgagaaacaagatgacttgggcaaaggtggaaatgaagaaagtacaaagactggaaatgctgggagccgcttggcctgtggagtgattgggattgcgcagtaaacattccctgtgtggtctgagtctcagactcatctgctaccctcaaaccattaaactgtaatctgaagagttgtaactgtgtgactcctttgactgggctaaggacagcaatgacagctgatggagactgtgtacaactcactgaattcaaatctgtttctgtgcctttccatattttgccagactacacaggtgataagctgaaattctcatttgagcctgttagtaaatatgtgtggcacttattttgagcctattaatgtgtacaaaaaaaaattttaagttagctctatacattgagcatcaataacagactcaatgatgctaactcatagtatttcattttgaaagtgttttatgtgataccatcaaaatggtgggtggtagcccaaacaaaatttgagcagaaaattttctgccccttatcagagaaattattgaaagctctcaagattcagagtacttaaccttatattttaaaattgtattaggattagatgtcatgatttaagaaaaagccctttagtaaacttgtatcaaactcatagaaggcaaacatggagcctcagctagctctactagccaagtgaagttggtaccacccatctttaaggttggcactcagggaaaacacaatagctcggggaatgacaccaagtttgactggaggttctggctaaatcgacttttatagccccaggtaatgaaattgagtgccttaatacccaagaaagagtgcctttgaaaggaaatattaacaggcttgtgactatctgaaatagttcaattgaagtattttcaacaaattgggtgtaaaccatagttctcactgatatactgaagtcactgaagaagagacaactaaattggaaaagcacataatttggtgtttccaaccttaaaatttttaagtttagatttccaatctaagattgctcataatgctttttcaagtagttatgttgaagttccaggtaaatcctatgtaactgatttccttaatgtagttttgatgtgggggatgactcaatgcggattaacttggtaatcacaaaccatttagtggctcacgtctcagtatttttagttggaaagacaagctgcaagtctgtccttggaatctgacattggatcatcgtcggatgcatgttttatgatactctaataaggacttaaaagcctaagtagggtcaccagaaagctgaagcctggcaaagctacagacacatttcttccatcattaggaagagctcagatctaaatgtcaaatgggaacatacaaaaaggaacttctaggtacgataaagctaagtttgacaagttttttgtttaacctagcaccttgtagttttaaaaatcatttttagggtgtgtgcactaagaggaaaacaagttcatattcttccaccttttattgtccc

SEQ ID NO: 8 - DNA, 19 bp, synthetic construct
  source 1..19: mol_type = other DNA
ttttttgcgcggtcctttc

SEQ ID NO: 9 - DNA, 22 bp, synthetic construct
  source 1..22: mol_type = other DNA
gagggaccagagagagcaagac

SEQ ID NO: 10 - DNA, 19 bp, synthetic construct
  source 1..19: mol_type = other DNA
cgccttccgtccgtcggct

SEQ ID NO: 11 - DNA, 16 bp, synthetic construct
  source 1..16: mol_type = other DNA
tgaggtcctgcactgg

SEQ ID NO: 12 - DNA, 33000 bp, Mus musculus
  source 1..33000: mol_type = genomic DNA
caattggatactgtcttagttagggttttactgctgtgagcaggcaccatgaccaaggcaactcttatgagaacaacatttaattgggactggcttacaggttcagaggttcagtccattatcatcaaggcaggatcatggcagcgtccaggcaggtgtgatctaggaagagctgagagttctacatgttcatctgaaggctgctaggcgaagactgacttccaggcagctaggatgagggtattaaacccatgcccacagtgacacacctactccagcctcttccctgacatttgaagttaggttctactgatgcaaggaataatagcggctataatcactcttatcaaaatggatattacatctcaaatagcatctgctcatatggtttaaatattattccatgtggattatcttttttttttattgctgagaactgtggttttattaattggttttttttttgtttgcgttttttgtttttgtttttgttttttttaatgtcaaatggtaaattttcattttattttatactttttaaaaattaggtattttcttcatttacatttcaaatgctatcccaaaagtcccccataccctctcccccccagtcccctacccacccactcccacttcttggccctggcattcccctgtactgaggcatataaaatttgcaagaccaaggggtctctcttcccaatgatggccaactaggccatcttctgctacatatgcagctagagacacgagctccgggggtactggttagttcatattgttgtttcacctatagggttgcagacccctttagttctttgggtattttctctagctccccatgtggattatcttaagagggtttcaatagtcacataagaaagtctctcattttaggggtgaagaccactttaaaaaaattcaaatggcttgctgaaagccacagacactagagaaataattatgtttagggtaaagtcggcttgtcctcttgtgaaactgtgtccaacggagacaagctgaggaattatgggcaatgataacatgttaaatgttgagctaaatgtattaaaaagcgcaaaaaaacaggtgtggtgggggaacagaggctagaagtctccctgaaaaaaatcaagtcttatttcctacttcccccgtgtttggtctggggaagaacaatagtgtttgcttaagcatctcattctgttttcaaaaccttgcatggttgacttgggaactgatagagaaaatgaagcaaacaatactctgtaatcaacattaacatcaaagctagctctctccactgtggtccctgacccaaccacgttaatctcccagggtcttgttaaaactgtaggcttgggagagaagtggttgtggtgaggggtaccttggagtaattctaagtctcttggggttaagcctggaagattggtgctttatgtattaagagtcctggggatccgaaagtagaaaaataatgatttttgttagtgactcagtatggttcttacttcaacacctcctcttaagggaggaagtagtgtgtgcatgtagctggaattttaaataataacagttcaggtgtgtgagtatttcattagacagtcagcttccatcctttatgcatcctttaagcaggttggtttagacaggtgcacaataaacgccaggctattaaacacagggactccaatatttaaaccaattcttttgaattgtctggctagaagcacatccagtctgataaagtagacaaagcacgtggagtcttgaaaagttcgacaccccttcggtcctcagatgactattatttagcaattgagagcactatactccttcttttgcccacacctataaggagatggacatggcacgtacatcccagcacttttgacagctttaaatggtgtttattacgagcaggtatggatgctgggaacagtgcttataccgagtcctggaaatgcttattaaacccaggagatacaaggagtctgccattaacctctctgtaactcaagagtagttatcaggagccaatgagggagaacaagagaattgaaacacgtgggaatatcggaccaccaggtagcgggtgtgtgtgtggggggaggcagccctagggagctctcaagccgaggcccctcggggtccccgcggcacgtggcgggctcgggccggagcgtcgcggcggtgggggaggggagcggcatgagccctggaggaggggtctagactgcccgggattcgtttgagcaggcgcggagtgcgcaggctcggcccaggcgcaaccagtgcgcgtaagaccgaggggtgaacctcactgctgcgcgtccgggtctctgagcaaggggctaaagtccgcttgtgcgcacctccggactgtgtgtgactacccgtagaaacactactgtcgccctggccggtggcgtccactccgtcccggtcgctccctcccgaccgtagttgtggtgcccccctccgcgcagccgcagtggtcgctcccgcctcgcagccccgccgcccggaggcctacccctgccgagtgtcgcgcgtcggcgccgctgcctatcccgctctgtgccctacggccctcccggcacccgccgccgtcccgctccgggctgcccttgctccgagggctgcagcaccggccgtccccgtcagccctcttgtctggccgccgggccgcggcgggcgggagcagccggaggaggagccgcagccgggaggcggcggcctgagcccatggcgtacagtcaaggaggcggcaagaagaaagtgtgctactactatgatggtgagtggccgaggagcctgcagccgggatgcggggagggcgcggcggggtgtcgggggtggcagcccgcggggacaccaacacggcctacccagcggtggcgtctcggggacgcccgccttcgctcctctcgggtcctctcgggcacacgcaggtttagggtgcactgcctctgaccggtctcccgaggaaccccagttggcaccctgggcctgactgcacaaagctggcgtcctggagctccgccccacgtccctgtcccctcccccgctctcccggtccccccttctcgcccccgatctgtccgcgctgcgcttccagcctcacccgggaccccggcacccgggtggggctgcggaggagttactgctggccggtggccaagttcgcagagcggcgctgtctcgctggtgttttgcgtggactacatccctcgcctttgttcgcgttcttcgagctccactttctcagggttcaaatggaagggctgcacttcctcggtgacaattaaggggttttgagttggaagtaggtaggtcgttgggaagtggtaggagagaggaagcgtgcagttgttcttgtttgatgggtcctggcatgtctctatgggaggttattacctcccccttctttgttgctctttgttcgatctgctataataccctgctctggggggttaggaaagaacaccggcatcttgcttaggaactcttctactgtggtgaagaccttgctgcaggaaactttgagaagtggactaaatattcacaatgcagtgaacggtacaaggtgactgtaaggattttctaatttggagtataagtagtaatagtctctgggttcagttacaccaaatggaatggtgccctgcctatacagatttcacccaagacatcaatagataaaccattgagagaagtaataaggaaatttgacagaactttcgaagctcattgattgaacgcttgatctccacaaagcaatatgaaattgggagcgttttctgtagttgtattgaagtcacagtgtttggagaggtagattagagaactaggctgaatgaaacacttaaacttgttaaaatagtcgtgcattttcttggtttgacagaatcttttaatacaagctttaaataaaatagccacctgtcattggctggaaaaccaaaaaccttacagaaagctaggccaaaatggaaaaggctgttttgttttttgagacaggctctaagttgacctggctgtcctgaaactcactgtgtaaatgaggctctaactcatagagatccacctgcctctgcctcccagatcctgggattaaagatatgtgccaccatggcctggcttaattggaagtttgagaggactcaaactgtattgcatgtttatttgaagttacaccatcatggagagcagagatgggagagcatgtcggtttaaacatgagttacattatgacgttggggtcatagcaaagccctgacctgcattctctgatccattggaaattacagcctttctaaagtaaagtcctgctcttgttagagtcaggggaaaggttgagctgttggggaacagctcagtctcttcagctgtcagcttctgtcatcagtgatgaactctaagtcccattaactttcaccaggcctgatggagaaccagagaggactctctgtcttgaacagctccttcctggcttcatgcgtagggatgagcctttgctgaggagtgggatagacattggtcatggagataaggtgtggagggaggcatttttcagtcatgtgattttcgacttatgcccatgcagctgctttacctagttgtgagtgaccaactgtataattgatggtggaaatctgctgagttatctatctgtggttcttacttgacttgtttttagaatgtacttaacttatttttattctgactatttgcatttgggggacgctaacaaaaattgaagatatataaattggtgtcatagttaagctaattaacatgtcatagcatgctagactacagagaattctctgcagagaattcccttcacgtctgacttgattacagaaaccctcttcttgttagtttataaaggtagcagcaatctttaattgtctttattaaattctaaacaagaggagcaagtaaagtgacttttgtaccaagaaagtgcatgactttcagacgcatgcagggttgggttggtggagaacccggccagtaggattggggctcgaatagtatactagtcttatgttagtgcttttaacttggttggtaggggaggagatcaggaaaggaggaggttaacggggcactaggcacccagagagcctcttgacaactcatgtgagtgttttcctgctaagactgtgtgcttagtgaagaatacttctgaagattgaatatggttttctccgtgtgtgctgcagtcttcagaattgtacttgtagggtctctgtgccactgatctgtgaactttcagataggagcgtatgaactatccctaaacctcacaagagaaatcagagttgttcttagtggactgtgactactgactggtcatcataactcccagaaacacatttcagaatgttggctaatctcttactaaactaaaagacaaagatcggtaaatgatataaagctaattatgtgtctacactgagatcctttctgacctctttcccgtttttctcaaagtgtaattgagcagatggggctacatttagttctaattgcaaggctcaggcttttaatactttatctagaaatataaactttgcctggtttagagtgaagcttgggaccaatcatgtagtgccttctaatgtatagtgtataacagaattgaatgtatgcataatttatgtaattagacaccaattgctttatgctttgtttattttctctgatagagataatcagaatcaaaatgtttggagacatagtccagaggaaagcacgtggcaacaaaaatatagaatataacaattactataattatatttcccagtgtactcctaagtttcttaacagatacaagagtactacagagtggccatcggttgtcagatttattttacttttatttaaaatgtgtgtgcttcttgtgtatgcctggtgcccttgatgtttaggagtggttgtgagctaccacgtagatgctggaaaccaaatttgggtcttctggaagagcaataaatgctcttaagctactctccagcctgtccgttcccactataacagtaggcaggaggatggttaggcttctgccacttatagatgaaaacaggtggacacttcatattctccacatgggttttctttccccacataggtttctgttttaatgtctaagaaagattcgtacctagaatacaaagattttatttttcttttgcagagttttgttcttctgtccctaaaaaaatgtattaataattggtttttttcttagcaaaattaaatgttagtaaattcttaaaacacatggagttggtgaacaaaaggaaaatttcttacattagcaaaatgaaatgtgaagtcattaagtaacggtttgggcttttttaaggccgccattgctatggataatgacaccgagatttatttctgtttagtaataaatgcctaggccttaagctaggcctactcccaactagctcatcactcaattatctcatttatacttctcagtttcctacaaccaacttcctccgagtccgaatagggaataccccacgcctaattctgagttcctctctcttccagatgtcccaccttactatcctgccttttgctgtaagccataggctttttattttaatctgtcaggaggtgccttaggcagttggggaaggatagagacacatcttcacacagtgtaccgaaacatcaccctaacaccattacattgaagttctactctcaccatgttggttagtcggtctcactctggtttcatggtttatgcttgtacatgaagtcagtttggggttcagaaacataagattgttttaagtccctgctttgtcccatatagacaaagattacttaagaaggaaatgggtgtttgtaacagagtaaaagaggaatggagtaataaaacaagcatctctaggaagcatatgcgccagtcttaaaacagtcctccctgtgaaacaacaaaggccaagtccttggaatgtgttgtacccaaaagaagggcaacccagagatagggatgctgggctgagccccaaggggccaggcacagcaagcttggttgaaagagagtttgactcttagagggagtgtcttggagcttttaaagtggaggctagtcaataagagaattggtgagaagtagaatgtaggctagaggaaagtgttaaagtgaaaggaaatggttgtctttagatagaatcgggtacatttgagagtggtttataaaccaagaaaaaaatacatcaaaactacttgttggtggcttggacgagggattagggctgggagttgtgatttctggactggtcatgagatggacaacatactgtccattgagattatatggaatcaagggctctatattggctattatgtatttgagatcttgatttagatcaaatgaaaatacttccaaaaattacctggaacccagggagaagtagtatttcttagctggtaagtgataaatactgacgtgtttgttgaaggtaatggtatggagagacacagactccggaaattgttcctgagaagtcataggatgaagtcactcttggtgtactgcttggccagtgacaagctcttgacttatgaggaaagtgaagaaaagctgagatttaaaaggtttggaggaagtgagcttagagaaggaacacaatggaggacagaattgtggagatccagactccacagtgtttcaaggaggagggagggtctaacagtgttaaaaccttcacagaactaatgttgtgtggcagacagaaattgcacagtagtctttttattcttgattttttcccctccctcgagcccccccacacacactacttttggcagcttctctatgtacagtgtgaaagtacatttcacagtcctgaaatttacaatgatctgaaattttgttcactcagaatcaaaagcttatcctgaaattaacgaaattacaatttattgtctttattccactaactgtgataattacacatttttgctttagaaatacctttgattatagaatgttgtcccagttttagtacattatatatttttatcttgagtcaaacttttctggatattcttaattggtaaaactatatttaagtatttgaacaaaaaatgggatcacattttaaatgcccaaattatgtaactgacagagactataggtacatgaaattaagaaattatgtaactgacagagactataggtacatgaaattaagaaattatgtaactgacagagactataggtacatgaaattaagaaattatgtaactggcagagactataggtacatgaaattaagaaattatgtaactgacagagactataggtacatgaaattaagaaaagaagagcagtgtgaggtttgatcttgctgtgcattgcaatactaagtgcaggccccaagatctggagtctgcacacagacctgagtaacagtatgtggccttgccttcaagttatttctgactggtaaataaagatgcctacagccaatagctgagcagaagagttgtatgtggggcttaggattcctattgtaaatataaaggttgtgtgtatcttttatctggaaactaaatggtcaaagccagggtagaaacgccaggttgggattaagcgttttaacaacagggctggatggcttagcagtttaaaaccctggctgctctttcagaggacctgggttggattcccagcactcacagggttgttcacaggggtcattcactacagccacaggggatctgatgctgtctcctgccctccatggccaccagacacagtcatatgtatggcaaaatcaccatgcacattaaataaaatttaaaaagaatcagttcagaaaacattttcttaaaaaagaaaaaggcagaaggttttttagtaagaaatagatgacttcaaagtcgccatgtataaaactaatagtgaacaggaaacattagaaatgaagattcttccctctcctcctcccagaatcacctaccctctgctgctagagtgctggtttaaaggtgccttgcccacaaaaatggttcttgtttgtttttgaaattataatatgatcacaccattttcttccttcctttactccctccaacccctcctgtgtagccttccacctgctctctttcaaatccatacccttggatttctttaactgttgatacatataaatactcctaaatacataaatacagtgttactatatgtttttagagctggctatttgttattggataaccaatgggttggtcattacctggaggagattctttgtcctatccttagtccttagttgtctgtagttctttgtctaggcttgaggcctcctgaccttcactaacgtgtctgttaatgaccttgttcaggtcatgtttagccagtcatgttggtgagactttgtggatgttgcttctgacatgtctcactgacaggcttacagtaaactccttgttcttcaagctctttaaaccctctctctcttccccaatgccctctgaccctaaggtgtaggggttgtattgtagatgtgtcacttggtactgggctccaaaactctgcatttcaattgtgcttttctgtaatggtctctgctacaaggagaacttttcctttataggggtgaggactacaggctcctgtgtttacaaggacaattatgtagaatgtagatcgggatgctgctgcttttgtaaagtagcaattgcatattttattcaaagatggatgacttcagtagtcttgagttggctaggtctccaatactaggcatgatttccctcttgctgaatggatctgaaatccaattagaaagctgttggttactgtaaaggtctgcgtgccaccactccacatttatgctgccatggtggttgttactgtggttcagaggtgtcataactgcgtaggactattggttgcttccctcctctggagccttgcatgactcctttttataatatgaagggtagtcctcaagaaggattaggctctcagttctgtccagctcaggggcttcttgggccctgcatctcaagtgcatgatgtcttcagcaatatgcagttacctctagggggcaaccaagggcaatagcgtataagattttgggagtctcttggatagtcctgaccagcaactccaaagacggctgcgtagttttttgtttgtttgtttgtttgttttgttttgttttgtttttgtggataatggctcctggagggagcctgtgtctatttatacacagtcttatgtgtattataggtacagtagggcaatggcatgattgtgcttgatccttgagacatcctcactgttcctctaccgtcctcattccttgtcctgtatttgtcttcctccctagttagaagccccactccattccccttacatttcctttccttccccttccttctccccctttgcgttcccgtcgctccccttttgtgcagaggaattaaaccaaaagcttgtacatgctagagaaatgttccaccactgtatatctcaagcctttggagggattcggaaaaattcatattatggctcagctgccctcgtgaatatgtgtcttttaggctaaatgaatattcttactgagaataaggcctcaaaattatgacagaagtttgtcgaaagctgtatatattaatataacgttaggagtctcatagttagaaggtaactcccttcataaattaggtaagccatccattttgttcatattcgtcaaatgaacaaattcgatgctgagcacaagcatgtatactgtattctttcctcattcgccattgtcctgctattacattgctgtgccagtgacaatacaaaagaatattcactgcctgtgctctcttcttcctaaatctgaatgtagctcctatctgctagttgtataattttggctacattatttaacatgcttctcggcagttataaagttgtggtacatcctagagttgagaaataaaagctgatgctgagtactaggaaaatgttcttgctgttacttctcaaacactacaacttaaagttggctgcatagggagaacatctggaaggattaggggaggggaaaataggacaaaaatatatttaaatttaaagttaaataataatataataaagaaagtttctacttaggtcaacaagatgtattgttttctggttctgaagttttcatttacctttgaaaaactagttagcattctgagtgctcctaaatttgtaaatcattttgtgaaaaaattgaactaaataaatcagaggtactataccaacagattcatactgtttgaaggcaggttttgtaaacctgaatgttcagctgggtctggtggcaaaagccagtggtctctcagtggaatcatgaattcaatgcctgcctgggacacatagctagaattttgtctcaagggggaacaaaagcaaatgttttctggattattgtcaagtagatagatagtatgaaaatttctggatttttgattgcccctatataagtgaaaaggtactatgagaggagagtttgaaatggggatgtttgtgtttgagagctggtcttgctgtgttgtctggccagcctggagtcctgtttgcaggtagataaggcttgccttgatcttacagagactcctgcttctgtttccctcagtgttagattaaaggtgtgcactaccataccctgcttaagctttgtccttataagggcagatatatgaagtgtgggggctgtcttttgttgtataaacctgtgctgaaacagtaagatctgcaggctgttaaagtcaggtcaactgtcctaacaaattatgaatatttgattttaaactataacattaatagtaatttctcatttcttgcttgataaggccattgtaaaattattccttatagggcaggagaaattactcagcagttaaaagcattggcagctcctactaaggacccagggttgtgttcctagcatatagccatctgtaactccatttccaggggatctgacacatcgtgaccaccacaggcaccaggtgtcaatgcaggctgcctatgtatatataggcaagctcgcaggcacataaaagtagatagtgtccctcaccttagaaaaagtaatacataagttttctaagcttgttgacaagctttcttattgtctaaaagtattttgtggttgaaaatcagattttggcattattctgtgtgttgttttaagacattggcatctgtcttacttagtttgggttttactgctgtgaacagacaccatgacctaggcaactcttacaaatgcaaacatttatttggggctagcttagtttcagaggtttagctcattatcatggtgggaagcatggcagcatgcaggtagacatagtgctggaagagggttctacatccttattcaaaggcagcagcaggagactccttcacaggcagccaggaggagggccttttccataccaggtagagcctgagcataggaggcctcaaaacccacctccatagtgacacactttctctaacaaggccccacctcctaataatgccacttcccatgggccaagcatattcaaaccaccacagcatctaagatgttttaatgcacaggctactactgtgtagtcctgagaaatgaagacaagagtgtctttattaccctgaaaaatgctgtgactctcccacttgtggacactgaacatttaagcccttccataattccagctcgactgtaaggtatttctacaggattccaataagttatccaagaagcactactagctgacaaattagatcctactccgtacttcaagaatacttcttatgtatctaaatttacaaaatgaacaacaacaacaacaaaaatccactaaaaatggatttcaccttaaggaaccaaaccagggaaattggaaaactaaaagtcagaaaaactttctctctccacaaagaatgtgaatcctaggcatgtataaaatctgcataatatattagatttctaatgtaatttgaatgttacaaaaacaactcttgtttaaatataaattttttgatgttgggtacatgtcagtggtggtatttaataagcatcttttctcttttaggtgatattggcaattattattatggccagggtcatcccatgaagcctcatagaatccggatgactcataacttgctgctaaattatggtttataccgaaaaatggaaatatatgtaagtactagttggcactgtgtttttaaactggtatttgaaagctcttcttaggctgctgtgggagatgcatgtggtagatgaaaagatctgaacgaacacagacaggtcttgtggttgtgtccctcagagaacgcttaaggaattggagtagtcctctcttccctctgatcatttcaaacaccaaatatttttgtgacaatactgttaagttgcctgtgctgctagaaacaattgtcctggggttggtgagatgacttaataggtagaggtgcttgccaccaactggatggcctgtttaccttccccagaactctcacagtagaaggagagacctaattcccccaaattgttttctaattccacgtgggcatgaggacacacctccaataataagtaaccacagtgtaattttaaaaagaaataaaagagaattgacctgaattattgcaagagtttggtagaaaattaatcataaatatttatttggttaacatacatatgaaatgttgccaatgataacaaggtaagtaaaaaatggtgtaagatacacagccacctaaaaagtcttcgaaacatgggcaggagcatacataaaaagtgcaaggtaggataaggcttaactaactactccaggagaagagggctagacagtgttataggaagcatttctttaagatacaggtattttagagaaatgggaagacttgtggaagaagtctgtggtaagtgaagctgagctcttcagagcagaagttagaataaacgaaggctaagggagatctcaagctggtgttcttagaaacctttgatgaaagtcagtgggtagacagcagcgaggctatgctgggaaagagcctgaacaaacaggagtagcctcgcttggagaggggctgccggctgcctgctgcctgccacaaatgtgtgcatttgaattagcattgtaacttgctactcaggtggatctggttcatttagagagactgatcgtggaaacttacacatatcatttgataattctcatttaacaactgatacttccaacatcctggtgtttttcttttcagaggcctcataaagccactgctgaagaaatgactaaataccacagcgatgagtatatcaagtttctacgatcaataagaccagataatatgtctgagtacagtaagcagatgcagagatgtacgttataaataattattttactagtgctgaatgtaaatgaatctttttaaagtttctgatcagagttgcctcaataggtatttttcctcataatttaaaatattaatataattattaattcagaaggtcattgaaccaatattaatgttactttagaaaacaaacctatttaaatttgttcttttactttttattttgcacccatgaaaagtttgggattggtagggagatcacagagttacaagagaaaaatatatttctttctttttttttttttttaataatttttttgagacagggtctgactttgtaaccagctgtcctggagattgctaatgcagatcaggctggcctcaaactcccagagttccacctgcttctctcttgcattaacaatcctccatttcaccttctgtgtactccagtcacattgttctgtaatgtaaagatgttgccttttgttggcttctttgtgtttctttttattcttctacatgtttttgattataggtgaaagtgtattgttattggtagtcaattgtattaactggtagtcaagtgtattaaagaaatattgccaagtatcctgaaactgtgggctgctgtcttttgaagcttaataccggagcccatttcctcagtgagaggtggatttagttcagtgatgacgaatgataaggatttctcaagttcactgaacaccatatttccctaccgtatgttatcgcatttggttgactcaagactggaataggacggacggatgctattacatctattgttttttgggttttttatttgggatttacacagtaatgggtagccagcccttgggtaaatgacaggatttgtttaaaccattatagtgcttgtttatattaaattctatttgagcataaaaaactaaaactttttttatagtatctgtaggtctgtaaaacccctgccctcttgctaacgtgtgatctgagaagtgaagaatgactagggatgggccacacacacttctgttgtgttttttcctctcagcacagggagccaaccatgtcatgaccaagtgacatgtcatctgtcttatgttccatgaaactgattcattcatggctgcttctgaagtcagtgttagccacagaaaaaaaacaaagtaacttaatattttgatactcactaaaacatgtttcggagtcagggacactgtgtgtgagggtcagtaagatgagttagaaaggggttgcagccacttaatctttaactcactatcatatttaaagagaaaactagatttgtgcctattttcatataggcttttgatttatgttgttggtgatggtggtggttgggtagagtgacttcacaaatttcctaataagatttggtaatggaattacatagataacaatttaattatcttgtaaaataagcattatattataaaattataacttttataatactacctacattgaagtactactccctgtgaattttaaaattcataggtaatattttaaaattacaaatttcacattgctaccatgaaatattatattattacctatgattgcctgaaacaaatatttttaatagtttataagaaaaagccttcaatgacttgataatagattgactttaatgaagttcaccttccacagttaacgtcggagaagattgtccggtgtttgatggactctttgagttttgtcagctctccacgggtggttcagttggtgagtatcctaaatcagtcagcctcaagaggatctgaaggggttagagtgtctgtaggttttgtctaaagcggaagttgtaatggtagtaaggttgtgttggcttgattttgctgtatcacccaggtgggcctccgtctcagccacctctgtggctacctcccaaatgcttgcttacagacatgtgctatcattccaacttaatgagtacccaagctgtcttgtagttcactgtggggcccgggctgaccttgatattgtggaaatcctcccctggagcctctcaatcattgggattagaggcttggaatattatgatattgctattcactcttctgttaatgtaacaaaacctgcggtaacttttaaaagaatggtttatcttggttcacagtttgggagcttaactggccctgtccttggggactgtgatagcacattatggtatgtgcatttgggagaaggccagtttacctcatggccgctgaatggagaaaagaggattccagtatccctttcaagcaagatcagatctccagtaaactaaaatcagatcttggctcttagagagtccacccccttccagtggtaccatactggtgactaaatgttccctggggttcttagaaggcatttcaggttcaaagtagcaagtataattgtctttaaaatgccagtgtttaacagtttttacttgaacacagtctttaagcttgtgtctttttactcagagacctttttctcctcttctttcttccgccatttttactaagactttgcatgtgcactgtgatttagttgggtaaactgtaggaaaatggttatctgaggaaagcttaggctccgaagttataatcctttgcttttgaatgccaaaccttttgtgcttacacatggcatatttaatagggcttcgcttatatgtattttgtgtaacctgattttttaaaattctgagtattttataaataacatagcatgtatctccttcttagctggggctgtgaaattaaaccggcaacaaactgatatggctgtcaattgggctggaggactacatcatgccaagaagtcagaagcatcagggttctgctatgttaatgatattgtgcttgccatcctcgaattacttaagtaagttaattcaaactgaattttccctgtgatcagatctcttaattgaaagaaaaaaatgattttaaagactatcaaataaatggtaatagattaatgctgagtcttccagggtttgttgtgagcccctgcagaagtgtgagaaatagaccactacagtgggagagcgagagggcagacagtgccgtgcctgagactgccggtaaatggtctctgctcatttaggtttgcagtcgtctaagctgattaaaaatggctgctagagatccggaaatgtgaatgctaaagtaacttgaagtcaagcctttgcaacttgtgttataagaagtttgtctggccacttagtagcgtggctgaccccagttctctcatccatcctctacacagacacacagaacacatagataatagacacacagtgatgcccaactgctgaaggggttttggaagttatccttcactgctttttcagaagtgtgaaggtccttaggagtgtggacacttgtgaaactagcttattttccactgttagctataatgctgcagtgagttgtatttcagctttgactgcagctgtgttctgtgggctttgagagtgatgctcttgccccgcatgtacatcccaagagttaacttcctgaccttaaataaacagcagctaagtgctgtcagtgtaacatatctgactcccccaaccgacagaaaccggaaatctccttcattgacttgaagcttcttccactggcttcagttctagagatctgctgctttttccttcatagcttagtcttgagtaagcccagttctgtacagttcttcacctggtaaatctgaacatgatttgattggtttggtaagcagctctctttgctcatctaaacacattgggcatttgatcatgtttgattttgaactttatgaagaagttctgctatgatgaagcccgctcgctgtttgcactattttagattttcttagtgttgcttttctgtgccttgagaacataaatgcctgggtttgatcatgccagcttacgttgtatgagcacagttacagcatctttaaataataaacaaaatgttttaagtcagccacagtgacaagataatcatttactaccttaaatggtgaatttaaatttcattttgtatgtattggtgttttgcctgcgtgtgtgtctatgcaccacatgtgtgcagtgcccatggaggacagaagaggccggtagatccccaggaactgctgtaagagatggttgtgagtcaccatgtgggtgctggggatttaacctggattctctagaaggcagccagtgctcctaactactagtccaactttccaacaccagtagctttttttaaatacagtttttttctatttgcactttgacatagggatggaactgatgtcattctatggtggagtgactacagaagctcatttgagtagttgctgtgactggtggtgtacagagcatcagggaatgatgaaggcacttgtgatctgtcacagtcatgaggtcttagtatgctgtagatgatgtgtaaaatgtgctcatctcgctgtaaaaggtgtttatggatatggacattggactgtctcagaactgtcatgttataaaatactctgagttttgttttattcttggaccattggaaaccggggagtggggtggcacaccggcatgccagaggcaggtaggtttgagatgagcctggatgagcagctatcaaaataaccacagggaaaaggaaacaacctatagttctgctcactccaaagctgttgaaagaataaaaataataacagcactatgttggttacagttgcccatagattattttataatgctgaaatttttttaattgatttattttcactttatgtcatttgtgttttgtttacacatgtgtctgtgtatgtgttggattacctggaactggagttacaggtaactgtgagcctgctgtgtgggtgctggggactgaacctgcggcctctggaagagcagccagtactgccaactgttgagccatctccagccctccctccctccctccatgttgagttgtgttgtttgtttgtttgagatagggttcctctgtgtagccatgggtctcctagaattttctctgtagaccaggctgacctcagactcaagagacccacctgcctgtacctcctgatggctgtgatgaaaggtgtatgtcactaagcccaacaattctgaaattgtgtaatgtagcccttggcataccctttaggtgtgagtacatagataatgtctgcccgtttttaaagtgtaaagacaagggtagacctcagagtacagtactcgattgtcttggtagcccgaggagacgtgacgtgctggggtctggatgctgagactgcgggggaagaagggtgagcattcgctgtaagatgaaggacctgcttccacattccggcttgcgctttcctttcaggtatcatcagagagtcttatatattgacatagacatccaccatggtgatggtgttgaggaagctttttatacaacagatcgcgtgatgaccgtctcattccataaatatggggaatactttcctggaacaggagacttgagggtaagactgagttctgtcagaataaatataagaagagcataggaggttgctaatttctggaagagccatgttgtcttagtcattttgtttgtgtatttgtgatgggacctcggcatctggtgtgtatggttttttttgtttgtttccggagatgtggcctcaccttagttgagcagttgttgggatcattgcttactgttaggatttgtcacatgtggacccactgaatgctgcttgttgttcagctgtgattactttgaatatgtagaaacgagggatcagaagtggtctcaactcagttaggtgtaagtgctgatcctaaaaagtttgctgtcctctccaggttgcataggttgtcttgggtagcaggggtggggtcacaatgggaatggacagtgttctataggtgtggggtacagattatcacactgtcttgtggtaggttgggagaggtcaggcagtgtctttatctgagatcacaggcatagagtcccagggtgtcttattaggtggtgttgaagactgattttgggctccctacctggggttgtgttctcaggactccatgttgccccagagtggcgcagtggatgtagaagctctgaggatccctccctacctatgggtctgggtcacagggctcctcactttgagaatggtggataagtgggctctacttttacatcttgtaaaattgtttcatgcctcaaactgaggtttgtaagaactttttagtttattttgtgtggtggggaggagcacatgtgtgccctgttacatgtgcaggagtcctcctgccatgggtggatcctggggatcaaaccgtggggatctcacttgttgggctttatctacccaggtatttcgccagcccaaactacagtgcttgtgtgttatctaaaaactgccttagagcttaaaacgtacttctacaacctagaatacccaagacacaatttacaaaactcatgaaactcaagaagaaggaagaccaaagtgtggacactttgttccttcttagaagggggaataaaatacccacggaaagagtttcagagacaaaattcagagcaaagactgaaggcatggccatccagagactgccccacctggggatccattccataaacaaccaccatacccagacattattacatataccaacaagattttgctgacaggaccctgatatagctgtctcctgtgaggctatgccagtccctggcaaatacagaagtggatgctcacagtcatctataggatggaacacagggcccccaatggaggagctagagaaagcacccaaggagctgaaggggtctgcaactctataggaggaacaacaatatgaactaaccagtacccccagagctcgtgtctctagctgcatatgtagcagaagaaggcctagtcggccatcactgggaggagaggcccttggtcttgggaagattatatgccccagtacaggggaatgccagtgacaggaagcaggaaatgggagtgggtgggtagggaagcagagggaggggggaggatataaggaatttttggagaggaaactaggaaaggggatagcatttgaaatgtaaatgaagaaaacatttaattttttcagaattgtgtgttacttggtcttttaatcattttaaaatatgtcagactttttgttatgaaatagtcttctaagatctacttttgtgttttaggatattggtgctggaaagggaaaatactatgctgtcaattttcccatgagagatggtatagatgatgaatcatatggacaaatttttaagcctgtaagtactgctttcagaaataaaatgggagttgtaaatatccttagatactaatgtgtcttattctgtggctagatcatctcaaaagtgatggagatgtaccagcctagcgcggtggtgctgcagtgtggcgcagactccctgtctggggacaggcttggttgtttcaatctaactgtcaaaggtaagcagttcacgttccccctggtgtggtgtttctcctccccaagaacttcccataaaagttttcattgctgagggctggagagatggttaagaacacatgttccttttgtagaagacttgggttggatcccagctcctacatgttggctcaaaatctagcactctcttctgatttgcatgcatgtggtacgcaccatacatgtttaaaaacatatttttaatttctcattgttaccttttgcttgccaactcgatgccaaactctatatttgaatttttagtggatttttatttgttgttgttgtttgtttttgttatgtggctgactagttaatttagttccccaagtcttacatgttatcatatttatgtttatgtatttatccatgagtagtttgttgccatgtcagcgccagcagtttttaatcagttttttcagaagacctgtaccttgttgtctgattcagttgctgttacagagtatgaggatttagcttggcgcactttattctatcatggttactctcttccttacccaaccctaagaacttcggtcgctgtggctagtgctcagcagcagtggtttttagcttagcagtcttctctactggcaagactcacttttttttcttctggatttttttgtttgcttagtttccatattccttattactaaactatgaatttctaatgtattctgctcagctatctcaggtgctctgtcactgagctcctcctgttcggacttagtgtcctcatgggtttaatgggacagagagctctctgcttctgctttattatgtctcctgatcagtgggcactgtatctaacttgctggtggaacacataagtgtcttcatgaggaaagaagcccaagtgtaaatgtgtaaggtggtgttgaaattctcaagtccttacgaagagccgaagtccacatgctgaatacaagtctcatattgctgttatgggatgaacttcgagggtttcagaatgaagcaaagttgcatgtggcagcgcatgcttttagactcagcattagaacagcagagatgggtccaggccagctaaggctttatatatagtaaaaccttgtctcataaagaaaaagataaataaaataaatctttttaataaagttaatgtttggattagaaatgacttagcatgcacattgcatgttttactttagactttctgtgtgaactttgaggtagtaagtaacatcttgctttgagttctcttacagaacttttaagataattaaatatagatgttaagaacttctcatgattgtactctgcagaccagcctcagcactgtggcatgctctggtactctccttcgtatctagttgggtcatacaaaattagttggccactctcctggaggtcagcattcagttaacagcactttcatgtgggccatagaatgttcatagatatctcgtgtttccgagcagataagacttaaccggaagtcatcagccaccaggtagttcctttctttttaggagctatatactgttagtgttctcttttgtaactgaaagtttataatgcttgtatttaaaagtagtagctttcattataaactgcatttgactctttatagtacatctgcattgtgtttcaacaggtagaagttcatttgtgaatttgcaattccattttcagacttagaatacactgctgcgactggcttcaactacattgtctcaggcctagtgaagccaagcagccagctgctgtccaccttgcctccttaattatttcttcttgtggtgttcttttccgtgcttcctcttacgggttatagccttcattgtataaaacccctttgtagtaatttttcctgctgttgggtggtagaatgctggtcttgtctgggctctttcattgtactctgaagttaacttctgtccttaaaaatgtccaagagacactgttgtagtagaagatgcctggttgataatgtacatgaagaaaacagagaaggcactataaactctcactaggtaatgaggatttttttttcttttgtggcattttctttttttccaaatattttaagctttgttatagaaaagttacaaaaacaaacaacaaaaataaaatcacacactgaaattaaccagacccaaagattcaatgacttttgtgcttagagcagccagcgtctcaataccaggtgaaagcccactgcaaaaggaaatgactttgttttttaaaaaaaaaaaaaatcttttttttaataaggctaaaatccatgaatctctgctgtcatcattgagggactttctttctttaatggttgaattatgtgcctgaatggcaccagaagttactggaaacttgtaacttgttcctgtgtgaggcttgtaatctgttttgttttttttttgttttttttttttttttttttggtttttcgagaaagggtttttctgtgtagccccggctgtcctggaactcactctgtagaccaggctggcctcgcactcagagatccgcctgcctctgctgggtgctgggattaaaggcggaggtttgtaatcttaaataaattaagaaaattatccacaataccagcaataaggatactctgtggtcaaactttttggtattgcttttcgaaattaagccacacatgtctgtgcgggagaaacaaaatttagcatttgtttattgtgttctctcccttatgtgatctctcatgaacgtcttctatggtagtaaaatacatgtgtgcgtattttaacacttagtacaagttattagttggatatacacccccccaggccccccaccctcgtcatcatcccctgtcattgttcctccctgccccccccacccctacccccccccccccaccttgtgttgaggtgcctgccttgtagtcatgctgtatagactggcctcccaattctgatccacgtaccttagcctcccaagagctgggattgctggtatgtgccactctgcctggctgtctacagacattttttggttgagtaaaattgtttgtatttatcctttttaaggtcctattttaaattgctaaagaatacatatttcattgttacccaaatcttctttgtctaaggtcatgctaaatgtgtagaagtagtgaaaacttttaacttgccattgctgatgctcggtggaggaggctacacaatccggaatgttgcccgatgttggacatatgagactgcagttgcccttgattgtgaaattcccaatggtaggtgttcaggttgcagtatctagaagaacatctgctatgtacaaatggatgcatgggagagtctactgccacacccctgaaatgtgtgatctcttctgatggatgagtggttagattgcaaatctgtgtgagagcattccatgtgcactttcaagctttcctttgggaatagttcttttatctggattcggattatgtttctgagattatacggaagctaagcttttaatgtgtaacttgttttttcattgtttttaatagagttgccatataatgattactttgagtattttggaccagacttcaaactgcatattagtccttcaaacatgacaaaccagaacactccagaatatatggaaaagataaagtaagaaatcacttcggcttaatgaaacttcaggaggctatagaaggtcaaataaaggaagttggtttagcatatacatcagatacttcctaaccttaggctattcctgttttttaatctcttatattaatacaaatatgtaacctttgtaaatagaaacattcttattagatcaaatgctttatgtctacagaatgtagaaacattgatcagaacgggctgtgtcctctctcccatagaccagttgtatgacatttataagtacacctcattgtcaaatagaggtgaaaaggtacatgtttgtgtgctgtgggttcagtcagctaatatatgcagtgatctgatgcttagatagtgtcccattcagtggttagtaaatgaaagctcgttcttgtttagttcctcctgtgtataacagaaactttacatacagtggattttgtctaataaattgtgtcatttagacagcgtttatttgaaaatctacgtatgttaccacatgcacctggtgttcaaatgcaagctattccagaggatgctgttcatgaagacagtggagatgaggatggagaagacccggacaaaagaatttccagtaagaaaacccttgctatgtcttcttgcatttttcttatgtgtcaaaataagacttaaaattgaaggtacacagggaatggttcacagcacatgttgtgattttccttctctccattttaattacatatagatttagctccctgatgtctcaaagcctgaattaatatcaccagtttcattttgtgtgactacacagacatggctgtgtccagaaagtaggcacttgatatatctatctatctatctgtcttacctatgcaatgattgtagaacctgtggctattatcaaatttataaaatcttttgtgtatcagttcgagcatcagacaaacggatagcttgcgatgaagagttttcagattctgaggatgaaggtgaaggaggtcgtaggaatgttgctgatcataagaaaggagcaaagaaggctaggattgaagaagacaagaaggagacagaggacaagaagacaggtcggtttatgttttggtgaccatttcactttccctacttaagggttgcactgtgtctcttagcgatcctgcagtcacacgtctcactttaggcaggtaacttttcctggtgtaagagatagttagttaccgctcatgcttactgtttagtgcttcagagctgacttaaaggttttacagttgtgctcaaattttctttgtggtatagaaaccttccttttaataacatagtagtaaacgttacatgtcatgggatggggttaaggggatgacagtagttacagatgttgggcctcagaacatttactgtagcttaggtggacttagactcagtgctcttctgccttgtcctcttatatactgggattgaaggcatgccagtacacctggctaaaattctaaataatttatactggttaaagctgacttcatgtagcaaaagttaagctacatagtcctttgaaaagttactttaagagtgaagactctttaggaactgaagaaactaaaactgaggaaagataggaaggggcagtttctggtgtcttccggtatttcattcagagtgtttatttagcatgccattacagcacctcgttagcactctcaggtttctcattgctatgctgaactgtgcaggggtaaggagtgggcagtagcctctttaaacatgataatgctgcaagttttagttgacctactagtcaagtgaggctgcctggctttactaggcctcactatatagacatggctgctccggctacttactgggtggccttggattcacaccattgtcctttctcagatgcctgggtgctgggattacagaaatgtgccaccacctccaactcagtttctttctattttaaaatatcttctgcattttcttttttttttccaaataggaaagtgataaagtaaattgactcttggtattttagacaaaataccacaataatctatgggtcataacctcaaataagacccaccaccaccccctcttggcactgagtaaaaatttagccagaattatccaagacactaaaatgtgaaatctccattgtcggcatgtagatttgtatgggaggtgacagcagtgccctgcagtgtctgcattgcccaagcgtgctgctgtccctgcacctctccatgaggccattgcttcataactaatttacacagagaaaatactctggctaatctgggacatttactttaattactgtatttaagttatgaagttcagtttagtagtaattaaaaatttaaggttagaacaatgggatattgtgggccagcaaggtaaaggtgcttgctgccaagcctgattacatatgatctgcatgttggaggaagactcctgacttctacaagctgtcttttgacctccctatggaagtcatggtgtggatgcccaaacatacacacacactctgaataaataaatgttaaaaacaaaaaacataatctagcctcttgcctttaggatcacgtccttcttacagtgcagacacagttctcttgagctccttctgtcttaccactgcttccgtgcctcagtccttttacagttttccctggaagataccctttcctcttacctgagagtttgttcattctttgggacccacattctagagaactgtgtctgcactgtaagaaggaagtgatcctaaaggcaagaagctaaattatttttttgtttttaacatttattatatatacactatatacatacatatatatattgtgtgtcttatatttcaaaatagacatcaatatttttcttctgtctttagatgttaaggaagaagacaaatccaaggacaatagtggtgagaaaacagaccccaaagggtgagcgatgcttgtgtctgtagacgtcatgcatgtcttgggggttgggcgggggcatgcatacttgagaaatactggcacagcctgagaacatacaatgagtgcaggtctgcgtgtgggtaacgtgcttaggctatttgaccggtaacttttatgtatgcaatcgtgtttttcatgatgctcatattactgtgcttagaatactttgatgttaggagacaggctagtttaaggaaaaaacattgggctaagtgaaagcctattggagaattttgttatcagaatgcatgtatggctaggtaggtgaagtgttgcggtaaatctccaacccaaatatgccctggcaatgaaacacaactcaattaatatgaatacatgctgtgtgcctagaatgggcagatctaccgctacactaccatcgtcaacaactgtgagagcccttagaacttgcagtttctccaggccacgtgcttctgctccacttttcttcttccccctcctctgcatcctcaccctcccctattttctcctctctctccccaccttcttctccaccttccctttatctgcccaatcatcagctctcctttattttacaaattaggtgggaagcaggtttatgggaagtaaacctgagtgctgactcattgcttgtttgtaggccctcactggagaaggaagtagcatcaaatataataagtaagccccagggctatccacacctgtgaagaggcttgagtctataatctcagtccactggagaggctgagcccagaggattgccatgaatttaaatccacactaagctacatagtgatttttaaatcgtcactctgggctgaagaatgagagtctgtgttagaagcaagcacgtgtgcttggtcacccagttgtttgctaattttgaaggataacttagaatgctcagttgtaaaacaactggaaagattaaggcttttattgctcagaaaaaaaagaaaaaacaacaacaacaacaacagtgaatagcttggtgtgaatctataagactgtgcaagtagccaggtcagtgctcctaaaccaagactattacttttaatatttcatgatcctctttcaacagagccaagtcagaacaactcagcaacccttgaatttgactctccaactttaggaacctcgaaaagtgagacgattctgggataagaaaccttccctgtttgaggacattggcttcattttatactgttttggcatggactgtatttattttcaaaatggcttgtttttgtttttcttggcaagttttattgtgagtttttctaattatgaagcaaatttttttttccaccatgctttatgtgattgtatttaaattgatgtgttattatgtcaaaagccggatctattaaagaaacaattggcctttctgagctgatttttccatcttttgtaattatctttattaaaaaattgtacttggatcgttttctgtctgtttattatgaaagcttgtttccaagtcaatgacttgatggtcttaagactggaacataccaaaaggaatgtcagtgtcagagaccatcactagatctacacagtgcttactggctctaacagctatttcttacttcaggaaaaataacagtgtcactttgtgtcaggaagactggtatttcataaattatttccaaattcataatctgtgcacttgggatgaagcaattatttttgaactgaatcaaatactcagaaattggaaattgtgaattgaaaaaagaaacctgtccaaccaataactggcccaacttaagacccatcccatgagccagcaccagtccctgacacttaatgatactctgttatacttgcagaccggagcctagtgtggctgtcctctgagaggctccacccagcagctgactaggacagatgcagatacccacagccaaacagtggatggagcttggggacttttatggaataggaggaaggatttcaggcctccaaggagataggaactctacaggaagaccaactgagtcaactagcctggacctttgtggctctcagagactgaaccactaaccaaagagcatacacgggctggacctaggtctacccacatagatgtagcagatgtgcagcttggtatttgtgtgagtcccagacaactggagaggaggctatgccaaaagcagttgcctgtctggatcttccagcttggctaccttgtctggcctcaatgggaaaggatgtgcctagcctcagacttgatgtgtcagggcatgtatacaaaagattgtaggcggtagaaacatacccttttgtgatgtcagtgtaaaatgaaccagctagcaaaacctggttcttgccctgtagtttgatcacaaccagggtttctactcaagaagtaaaacttaatatgctttggcattccgcattcattgagtgtctactcaccttttggtaataactgcagctgaatagaattttaagtgctttcacctaaacttagattgttattgtagaaagcccacaaatggagtttcctctcccagtgtgtacaacagaaaaatgttttaggaatttgccaaagttggtgaaaccatcctacttgctagaatcaggttgctgattttgagattaggagaacagagtggtagaaaggaggcagtcgtggagaatgttcttacattgtcctgaggttcgaaggtttcatgttcatgggttttccccttttgctatgtatttgccaggagtccatatcctttatgactaaggaagcaggatcccttcgctgcagagacttatttagtgtgtaattttcatgaaacaaagtgtatcctttggatacttcatctggtattgtgatctgccttctggcacatgtcgcataataattacagtgtgttaagttctggtctatgaatctactagaatgatgtcccagatgccacagtgattctccttggttggatctgggaggttctggtttcatagtttaaggctgtgaaactaagcagtttcagaacaggtgatttgggacggtaaaaatacacagataccagtaatttgtaaagtcaaactacagcccacattgtttggaagaacaaaggctccttttgcatagcagtaatcacaatttctacaaatgtgtaaaactgattctatgcataatgaataagatgagtgttaataatagcctgtcgggacaagggttgaaaatcaaaacaatgaggaaataacaagaaccaggtggagggaggcgaagttcatttttctgtcacaagagtgagatttataaatagaaaagacaacaaaacagaacctggtaaaagccatgggaatttttcaaaagtgaaggccaaagcttgacaccgaaccccacataagttacatacttaacagaagttctctagaccagcaaaaacaagtaaaataagatagggaaccccggcagtggtggcgcactcctttaatcccagcacttgggaggcagaggcaggtggattcctgagtctacagagtgagttccaggacagcctggactgcacagagaaaccctgtctcggaaaacccaaaaaaaaaaaaaaaaaaaaaagatatgggaaccataatggacttgttccacctcaaagaactttatttctttattttaaaaataaagggtctttgtttttggccaggggtagagagaaaattcagtggttaggagtatttgctgctcttacacagggtccaggtttgattccaaccacatgctcacagccatctgaggatctgacacccctttctttgagtaccaggtatacatgtataaaacacccatacatgttaactaaaaatagcttaaaaatttgacaagagttcaatgaaacccatgaatgtagtatcttgttaggaagacacaatgtccaaggtaattcttataaaggaaaacatttaattgaggctggctttccatcgcataggttcagtccattatcgtgatgggaagcatggcttgcagatagacatggtcttgatcagcagggaacaggaggagactgttccacactcggccgaacttgagcataggagacctcaaagcctgcccccacagtgacacacttcctctaacaaagccatacctacaacaagaccactaatagtgccactacctatgggccaaacatttaaacacatgtctaggggtgggggttagtacctattcaaaccactacattccaccttctgggccccatagtcttgtacctttaccataatgcaaaatgaatttaatttcaaaagtccacatagtttagcacagtttcagcagtgtttaagtccaaagtctcttctgagattcatgcactcttttttattagatattttcttcatttacatttcaaatgctattctgaaagtacccaataccctccccccccaactcccccccccccccgccctgctccccaacccactcacttctgcttcctggccctggcattaccctgtactggggcatataatctttgtaagaccaagggcctctcctcccaattatggccgactaggccatcctctgctacacattcatgtactctcttaac

SEQ ID NO: 13 - DNA, 16 bp, synthetic construct
  source 1..16: mol_type = other DNA
tagtctctgtcagtta

SEQ ID NO: 14 - DNA, 16 bp, synthetic construct
  source 1..16: mol_type = other DNA
tcatgtacctatagtc

SEQ ID NO: 15 - DNA, 23 bp, synthetic construct
  source 1..23: mol_type = other DNA
tgatggtgttgaggaagcttttt

SEQ ID NO: 16 - DNA, 21 bp, synthetic construct
  source 1..21: mol_type = other DNA
tccctcaagtctcctgttcca

SEQ ID NO: 17 - DNA, 26 bp, synthetic construct
  source 1..26: mol_type = other DNA
acaacagatcgcgtgatgaccgtctc

SEQ ID NO: 18 - DNA, 16 bp, synthetic construct
  source 1..16: mol_type = other DNA
tgcagtggggtgattt

SEQ ID NO: 19 - RNA, 5292 nt, Homo sapiens
  source 1..5292: mol_type = mRNA
ctcttgcagtgaggtgaagacatttgaaaatcaccccactgcaaactcctccccctgctagaaacctcacattgaaatgctgtaaatgacgtgggccccgagtgcaatcgcgggaagccagggtttccagctaggacacagcaggtcgtgatccgggtcgggacactgcctggcagaggctgcgagcatggggccctggggctggaaattgcgctggaccgtcgccttgctcctcgccgcggcggggactgcagtgggcgacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggttctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccgacctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcgtgaacatctgcctggagtcccgtccctgcccagaacccttcctgagacctcgccggccttgttttattcaaagacagagaagaccaaagcattgcctgccagagctttgttttatatatttattcatctgggaggcagaacaggcttcggacagtgcccatgcaatggcttgggttgggattttggtttcttcctttcctcgtgaaggataagagaaacaggcccggggggaccaggatgacacctccatttctctccaggaagttttgagtttctctccaccgtgacacaatcctcaaacatggaagatgaaaggggaggggatgtcaggcccagagaagcaagtggctttcaacacacaacagcagatggcaccaacgggaccccctggccctgcctcatccaccaatctctaagccaaacccctaaactcaggagtcaacgtgtttacctcttctatgcaagccttgctagacagccaggttagcctttgccctgtcacccccgaatcatgacccacccagtgtctttcgaggtgggtttgtaccttccttaagccaggaaagggattcatggcgtcggaaatgatctggctgaatccgtggtggcaccgagaccaaactcattcaccaaatgatgccacttcccagaggcagagcctgagtcactggtcacccttaatatttattaagtgcctgagacacccggttaccttggccgtgaggacacgtggcctgcacccaggtgtggctgtcaggacaccagcctggtgcccatcctcccgacccctacccacttccattcccgtggtctccttgcactttctcagttcagagttgtacactgtgtacatttggcatttgtgttattattttgcactgttttctgtcgtgtgtgttgggatgggatcccaggccagggaaagcccgtgtcaatgaatgccggggacagagaggggcaggttgaccgggacttcaaagccgtgatcgtgaatatcgagaactgccattgtcgtctttatgtccgcccacctagtgcttccacttctatgcaaatgcctccaagccattcacttccccaatcttgtcgttgatgggtatgtgtttaaaacatgcacggtgaggccgggcgcagtggctcacgcctgtaatcccagcactttgggaggccgaggcgggtggatcatgaggtcaggagatcgagaccatcctggctaacacgtgaaaccccgtctctactaaaaatacaaaaaattagccgggcgtggtggcgggcacctgtagtcccagctactcgggaggctgaggcaggagaatggtgtgaacccgggaagcggagcttgcagtgagccgagattgcgccactgcagtccgcagtctggcctgggcgacagagcgagactccgtctcaaaaaaaaaaaacaaaaaaaaaccatgcatggtgcatcagcagcccatggcctctggccaggcatggcgaggctgaggtgggaggatggtttgagctcaggcatttgaggctgtcgtgagctatgattatgccactgctttccagcctgggcaacatagtaagaccccatctcttaaaaaatgaatttggccagacacaggtgcctcacgcctgtaatcccagcactttgggaggctgagctggatcacttgagttcaggagttggagaccaggcctgagcaacaaagcgagatcccatctctacaaaaaccaaaaagttaaaaatcagctgggtacggtggcacgtgcctgtgatcccagctacttgggaggctgaggcaggaggatcgcctgagcccaggaggtggaggttgcagtgagccatgatcgagccactgcactccagcctgggcaacagatgaagaccctatttcagaaatacaactataaaaaaataaataaatcctccagtctggatcgtttgacgggacttcaggttctttctgaaatcgccgtgttactgttgcactgatgtccggagagacagtgacagcctccgtcagactcccgcgtgaagatgtcacaagggattggcaattgtccccagggacaaaacactgtgtcccccccagtgcagggaaccgtgataagcctttctggtttcggagcacgtaaatgcgtccctgtacagatagtggggattttttgttatgtttgcactttgtatattggttgaaactgttatcacttatatatatatatatacacacatatatataaaatctatttatttttgcaaaccctggttgctgtatttgttcagtgactattctcggggccctgtgtagggggttattgcctctgaaatgcctcttctttatgtacaaagattatttgcacgaactggactgtgtgcaacgctttttgggagaatgatgtccccgttgtatgtatgagtggcttctgggagatgggtgtcactttttaaaccactgtatagaaggtttttgtagcctgaatgtcttactgtgatcaattaaatttcttaaatgaaccaatttgtctaaaaaaaaaaa

SEQ ID NO: 20 - RNA, 16 nt, synthetic construct
  source 1..16: mol_type = other RNA
tgcagtggggtgattt

SEQ ID NO: 21 - RNA, 23 nt, synthetic construct
  source 1..23: mol_type = other RNA
ataaaatctacagtcataggaat

SEQ ID NO: 22 - RNA, 1435 nt, Homo sapiens
  source 1..1435: mol_type = mRNA
ggcggggcctgcttctcctcagcttcaggcggctgcgacgagccctcaggcgaacctctcggctttcccgcgcggcgccgcctcttgctgcgcctccgcctcctcctctgctccgccaccggcttcctcctcctgagcagtcagcccgcgcgccggccggctccgttatggcgacccgcagccctggcgtcgtgattagtgatgatgaaccaggttatgaccttgatttattttgcatacctaatcattatgctgaggatttggaaagggtgtttattcctcatggactaattatggacaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtaatgaccagtcaacaggggacataaaagtaattggtggagatgatctctcaactttaactggaaagaatgtcttgattgtggaagatataattgacactggcaaaacaatgcagactttgctttccttggtcaggcagtataatccaaagatggtcaaggtcgcaagcttgctggtgaaaaggaccccacgaagtgttggatataagccagactttgttggatttgaaattccagacaagtttgttgtaggatatgcccttgactataatgaatacttcagggatttgaatcatgtttgtgtcattagtgaaactggaaaagcaaaatacaaagcctaagatgagagttcaagttgagtttggaaacatctggagtcctattgacatcgccagtaaaattatcaatgttctagttctgtggccatctgcttagtagagctttttgcatgtatcttctaagaattttatctgttttgtactttagaaatgtcagttgctgcattcctaaactgtttatttgcactatgagcctatagactatcagttccctttgggcggattgttgtttaacttgtaaatgaaaaaattctcttaaaccacagcactattgagtgaaacattgaactcatatctgtaagaaataaagagaagatatattagttttttaattggtattttaatttttatatatgcaggaaagaatagaagtgattgaatattgttaattataccaccgtgtgttagaaaagtaagaagcagtcaattttcacatcaaagacagcatctaagaagttttgttctgtcctggaattattttagtagtgtttcagtaatgttgactgtattttccaacttgttcaaattattaccagtgaatctttgtcagcagttcccttttaaatgcaaatcaataaattcccaaaaatttaaaaaaaaaaaaaaaaaaaaaa

SEQ ID NO: 23 - RNA, 21 nt, synthetic construct
  source 1..21: mol_type = other RNA
tcctatgactgtagattttat

SEQ ID NO: 24 - RNA, 21 nt, synthetic construct
  source 1..21: mol_type = other RNA
  modified_base 1: mod_base = OTHER; note = thymine
ttatctataatgatcaggtaa

SEQ ID NO: 25 - RNA, 19 nt, synthetic construct
  source 1..19: mol_type = other RNA
acctgatcattatagataa

SEQ ID NO: 26 - RNA, 8515 nt, Homo sapiens
  source 1..8515: mol_type = mRNA
gttctctcctctcggaagctgcagccatgatggaagtttgagagttgagccgctgtgaggcgaggccgggctcaggcgagggagatgagagacggcggcggccgcggcccggagcccctctcagcgcctgtgagcagccgcgggggcagcgccctcggggagccggccggcctgcggcggcggcagcggcggcgtttctcgcctcctcttcgtcttttctaaccgtgcagcctcttcctcggcttctcctgaaagggaaggtggaagccgtgggctcgggcgggagccggctgaggcgcggcggcggcggcggcacctcccgctcctggagcgggggggagaagcggcggcggcggcggccgcggcggctgcagctccagggagggggtctgagtcgcctgtcaccatttccagggctgggaacgccggagagttggtctctccccttctactgcctccaacacggcggcggcggcggctggcacatccagggacccgggccggttttaaacctcccgtgcgccgccgccgcaccccccgtggcccgggctccggaggccgccggcggaggcagccgttcggaggattattcgtcttctccccattccgctgccgccgctgccaggcctctggctgctgaggagaagcaggcccagtcgctgcaaccatccagcagccgccgcagcagccattacccggctgcggtccagagccaagcggcggcagagcgaggggcatcagctaccgccaagtccagagccatttccatcctgcagaagaagccccgccaccagcagcttctgccatctctctcctcctttttcttcagccacaggctcccagacatgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctgaatttttttttatcaagagggataaaacaccatgaaaataaacttgaataaactgaaaatggacctttttttttttaatggcaataggacattgtgtcagattaccagttataggaacaattctcttttcctgaccaatcttgttttaccctatacatccacagggttttgacacttgttgtccagttgaaaaaaggttgtgtagctgtgtcatgtatatacctttttgtgtcaaaaggacatttaaaattcaattaggattaataaagatggcactttcccgttttattccagttttataaaaagtggagacagactgatgtgtatacgtaggaattttttccttttgtgttctgtcaccaactgaagtggctaaagagctttgtgatatactggttcacatcctacccctttgcacttgtggcaacagataagtttgcagttggctaagagaggtttccgaagggttttgctacattctaatgcatgtattcgggttaggggaatggagggaatgctcagaaaggaaataattttatgctggactctggaccatataccatctccagctatttacacacacctttctttagcatgctacagttattaatctggacattcgaggaattggccgctgtcactgcttgttgtttgcgcatttttttttaaagcatattggtgctagaaaaggcagctaaaggaagtgaatctgtattggggtacaggaatgaaccttctgcaacatcttaagatccacaaatgaagggatataaaaataatgtcataggtaagaaacacagcaacaatgacttaaccatataaatgtggaggctatcaacaaagaatgggcttgaaacattataaaaattgacaatgatttattaaatatgttttctcaattgtaacgacttctccatctcctgtgtaatcaaggccagtgctaaaattcagatgctgttagtacctacatcagtcaacaacttacacttattttactagttttcaatcataatacctgctgtggatgcttcatgtgctgcctgcaagcttcttttttctcattaaatataaaatattttgtaatgctgcacagaaattttcaatttgagattctacagtaagcgttttttttctttgaagatttatgatgcacttattcaatagctgtcagccgttccacccttttgaccttacacattctattacaatgaattttgcagttttgcacattttttaaatgtcattaactgttagggaattttacttgaatactgaatacatataatgtttatattaaaaaggacatttgtgttaaaaaggaaattagagttgcagtaaactttcaatgctgcacacaaaaaaaagacatttgatttttcagtagaaattgtcctacatgtgctttattgatttgctattgaaagaatagggtttttttttttttttttttttttttttttaaatgtgcagtgttgaatcatttcttcatagtgctcccccgagttgggactagggcttcaatttcacttcttaaaaaaaatcatcatatatttgatatgcccagactgcatacgattttaagcggagtacaactactattgtaaagctaatgtgaagatattattaaaaaggtttttttttccagaaatttggtgtcttcaaattataccttcaccttgacatttgaatatccagccattttgtttcttaatggtataaaattccattttcaataacttattggtgctgaaattgttcactagctgtggtctgacctagttaatttacaaatacagattgaataggacctactagagcagcatttatagagtttgatggcaaatagattaggcagaacttcatctaaaatattcttagtaaataatgttgacacgttttccataccttgtcagtttcattcaacaatttttaaatttttaacaaagctcttaggatttacacatttatatttaaacattgatatatagagtattgattgattgctcataagttaaattggtaaagttagagacaactattctaacacctcaccattgaaatttatatgccaccttgtctttcataaaagctgaaaattgttacctaaaatgaaaatcaacttcatgttttgaagatagttataaatattgttctttgttacaatttcgggcaccgcatattaaaacgtaactttattgttccaatatgtaacatggagggccaggtcataaataatgacattataatgggcttttgcactgttattatttttcctttggaatgtgaaggtctgaatgagggttttgattttgaatgtttcaatgtttttgagaagccttgcttacattttatggtgtagtcattggaaatggaaaaatggcattatatatattatatatataaatatatattatacatactctccttactttatttcagttaccatccccatagaatttgacaagaattgctatgactgaaaggttttcgagtcctaattaaaactttatttatggcagtattcataattagcctgaaatgcattctgtaggtaatctctgagtttctggaatattttcttagactttttggatgtgcagcagcttacatgtctgaagttacttgaaggcatcacttttaagaaagcttacagttgggccctgtaccatcccaagtcctttgtagctcctcttgaacatgtttgccatacttttaaaagggtagttgaataaatagcatcaccattctttgctgtggcacaggttataaacttaagtggagtttaccggcagcatcaaatgtttcagctttaaaaaataaaagtagggtacaagtttaatgtttagttctagaaattttgtgcaatatgttcataacgatggctgtggttgccacaaagtgcctcgtttacctttaaatactgttaatgtgtcatgcatgcagatggaaggggtggaactgtgcactaaagtgggggctttaactgtagtatttggcagagttgccttctacctgccagttcaaaagttcaacctgttttcatatagaatatatatactaaaaaatttcagtctgttaaacagccttactctgattcagcctcttcagatactcttgtgctgtgcagcagtggctctgtgtgtaaatgctatgcactgaggatacacaaaaataccaatatgatgtgtacaggataatgcctcatcccaatcagatgtccatttgttattgtgtttgttaacaaccctttatctcttagtgttataaactccacttaaaactgattaaagtctcattcttgtcattgtgtgggtgttttattaaatgagagtttataattcaaattgcttaagtccattgaagttttaattaatgggcagccaaatgtgaatacaaagttttcagtttttttttttcctgctgtccttcaaagcctactgtttaaaaaaaaaaaaaaaaaaaaacatggcctgagagtagagtatctgtctactcatgtttaattaaggaaaaacacttatttttagggctttagtcatcacttcataaattgtataagcacattaaatagcgttctagtcctgaaaaagtccaagattcttagaaaattgtgcatatttttattatgacagatgtttgaagataattccccagaatggatttgatactttagatttcaattttgtggcttttgtctattattctgtactctgccatcagcatatggaaagcttcatttactcatcatgacttgtgccatataaaaattgatatttcggaatagtctaaaggactttttgtacttgaatttaatcatgttgtttctaatattcttaaaagcttgaagactaaagcatatcctttcaacaaagcatagtaaggtaataagaaagtgtagtttgtacaagtgttaaaaaaataaagtagacaatgttacagtgggacttattatttcaagtttacattttctccatgtaattttttaaaaagtaaatgaaaaaatgtgcaataatgtaaaatatgaagtgtatgtgtacacacattttatttttcggtatcttgggtatacgtatggttgaaaactatactggagtctaaaagtattctaatttataagaagacattttggtgatgtttgaaaaatagaaatgtgctagttttgtttttatatcatgtcctttgtacgttgtaatatgagctggcttggttcagtaaatgccatcaccatttccattgagaatttaaaactcaccagtgtttaatatgcaggcttccaaaggcttatgaaaaaaatcaagacccttaaatctagttaatttgctgctaacatgaaactctttggttcttttatttttgccagataattagacacacatctaaagcttagtcttaaatggcttaagtgtagctattgattagtgctgttgctagttcagaaagaaatgtttgtgaatggaaacaagaatattcagtccaaactgttgtaaggacagtacctgaaaaccaggaaacaggataatggaaaaagtcttttaaagatgaaatgttggagccaactttcttatagaattaattgtatgtggctatagaaagcctaatgattgttgcttatttttgagagcatattattcttttatgaccataatcttgctgtttttccatcttccaaaagatcttccttctaatatgtatatcagaatgtgggtagccagtcagacaaattcatattggttggtagctttaaaaagtttgtaatgtgaagacaggaaaggacaaaatagtttgctttggtggtagtactctggttgttaagctaggtattttgagactacttccccatcacaacaacaataaaataatcactcataatcctatcacctggagacatagccatcgttaatatgttagtgactatacaatcatgttttcttctgtatatccatgtatattctttaaaaatgaaatttatactgtacctgatctcaaagctttttagcttagtatatctgtcatgaatttgtaggatgttccattgcatcagaaaacggacagtgatttgattactttctaatgccacagatgcagattacatgtagttattgagaatcctttcgaattcagtggcttaatcatgaatgtctaaatattgttgacattaggatgatacatgtaaattaaagttacatttgtttagcatagacaagcttaacattgtagatgtttctcttcaaaaatcatcttaaacatttgcatttggaattgtgttaaatagaatgtgtgaaacactgtattagtaaacttcatcacctttctacttccttatagtttgaacttttcagtttttgtagttcccaaacagttgctcaatttagagcaaattaatttaacacctgccaaaaaaaggctgctgttggcttatcagttgtctttaaattcaaatgctcatgtgacttttatcacatcaaaaaatatttcattaatgattcacctttagctctgaaaattaccgcgtttagtaattatagtgggcttataaaaacatgcaactctttttgatagttatttgagaattttggtgaaaaatatttagctgagggcagtatagaacttataaaccaatatattgatatttttaaaacatttttacatataagtaaactgccatctttgagcataactacatttaaaaataaagctgcatatttttaaatcaagtgtttaacaagaatttatattttttattttttaaaattaaaaataatttatatttcctctgttgcatgaggattctcatctgtgcttataatggttagagattttatttgtgtggaatgaagtgaggcttgtagtcatggttctagtgtttcagtttgccaagtctgtttactgcagtgaaattcatcaaatgtttcagtgtggttttctgtagcctatcatttactggctatttttttatgtacacctttaggattttctgcctactctatccagttgtccaaatgatatcctacattttacaaatgccctttcagtttctattttctttttccattaaattgccctcatgtcctaatgtgcagtttgtaagtgtgtgtgtgtgtgtctgtgtgtgtgtgaatttgattttcaagagtgctagacttccaatttgagagattaaataatttaattcaggcaaacatttttcattggaatttcacagttcattgtaatgaaaatgttaatcctggatgacctttgacatacagtaatgaatcttggatattaatgaatttgttagtagcatcttgatgtgtgttttaatgagttattttcaaagttgtgcattaaaccaaagttggcatactggaagtgtttatatcaagttccatttggctactgatggacaaaaaatagaaatgccttcctatggagagtatttttcctttaaaaaattaaaaaggttaattattttgacta

SEQ ID NO: 27 - DNA, 20 bp, synthetic construct
  source 1..20: mol_type = other DNA
tgcatggtgtagcccccctg

SEQ ID NO: 28 - DNA, 20 bp, synthetic construct
  source 1..20: mol_type = other DNA
gttttcaaacacaccttcat

SEQ ID NO: 29 - skipped

SEQ ID NO: 30 - RNA, 21 nt, synthetic construct
  source 1..21: mol_type = other RNA
cattttaatcctcactctaaa

SEQ ID NO: 31 - RNA, 23 nt, synthetic construct
  source 1..23: mol_type = other RNA
tttagagtgaggattaaaatgaa

SEQ ID NO: 32 - DNA, 141001 bp, Homo sapiens
  source 1..141001: mol_type = genomic DNA
aatttataaaggaaaaaggtttaattgactcacagttcagcatgtctggggaagtgttaggaaacttacaatcatggcagaagagaaagcaaaccatcctttctcacatggtgacaggaagagcaaagcggggtaagccccttacaaaaccaccagatctcatgagaactcactatcacgagaacaccatggaggtaactgcccccatgattcaattacctcccaccaggtccctcccacgacatgtggggattatgcgaactccaactcaagatgagatttgggtggggacacagccaaaccatatcagaagcttaaccttctttggagcatgattattcagttgaacctaagttcagtagtcacccagttatgctgtcttcagctactattttccatatgtttctcaaacatctgatatatcacactggctagtgcactttcttccaccagcataccatctcaatttaccactttaacaattggactgccactttgtgtcagggactatctgtgctccaactactacaagtgataaggtcctcactgacagccagggagcaagtgatccagctctaaaactcaccttatcatctgctttcctagaccactcctaacaaccaactattctgggttgagttctccaagaggcagagagttcaggatacagaatgttgttttgtttttgttgttgttgctgttgttgtttgtgtgtgtgtttgggcttttttgagacggagtctcactctgttgcccaggtagaagtgcagtggcatgatctcagctccctgcaacctccacctcctgggtttaagtgattcccctgcctccacctcctgagtagctgggactacaagtgtgcgccaccacacccagctaatttttgtgtttttagtagaaatggggttttaccatgttggctaggctgctcccaaactcctgacctccagtgatccacctacctctgcctcccaaagtgctgggattacaggcgtgagccaccacacccagcccagaatgtttattagaatgcacaattaataccagaggcagtggggaaggaaggactgagcagaggaggaagttgagttgtgattcaacccaacaactgcctggctggcatggggagctctggagttaaatagggccatcagactttcccagtgtggggccaacatgactgggtctttatacccccacctctgtcagtcactcaacgtggtctccctgcaacaaggtgactcttgcagccgagacaatccctgaagggacagagggctgaagcctgtctgccaacagcactcccagtggctggaacaagtccttccctataggggaatctgggcggcacacctccatctccatgtccatcacatacgatatcacagacatttaaatattttgataactgtacataagagtttcctttataatcttatagatcttattttatgcatttgaaaatattcttctgagacagggcttttatcatattgccatagggtgccacgatataaaaaaggttaaatactctctgattcagaagtatccaatgatgacttctctctcatgcatttaattgaaaatctggtttttctccttctctgctagttctctacctctctccccacctcccacatcatagcctattcacatatgtctgaatctcatgatagacaagttcaggttcttttcccaggttctttttaccacatccccccacccccacataaaaagtatatatggcacagcctaggttccacccaaatcctttctcctcttcttcctgggcccacaactctcctacatacattggtataccttgcgcttagggatggccatgtgactaagttctaacagtggaacatgatcagatgccacttccagcctctaagacagccagtgtgtttcctccataagctccttctcttcctcccaactggagactctaaatgatgaccctgcctcaagcaagcaaacaacaagtccctcaggggtggtgtaggctgcaaatggaaggagcttgagtcccaaaccttccacggagaaggctggctaccaacctggatcactcacccaagactgctcgaagagttggtttgaaccattgtgttttggggtctatttattacaacagtttagcttgctttgtgaatagatttagtggcagagcctccaaattctatagatacattgatctcagtcctaaccgcatctggaacaccattaaataaaggaattgcaaacccagagaaggtaatgaatttgtctaaggtcatacaagatggctaggatcaggacccaactctccagttttctttcttctctgctattctgccttctgtgatcctacataagtgggcatgattgtataacatatgcggccatgagatttctctttcagcaagagaaagggacaggaagaaagagagggaatgcattttcttggcctgaattagtgtgagccattagttacctacattgactaaattatctggaatgaacattcaactctacatcacatatagttaaaatgacagatctgcttaagattgtttctagcatacgttatttcaatttaggcaaatgtgaccattcagtgtgaggggaccatactgtcattaggtccctgtcagttctcaattatactgttatcttagagggggaaaaatgtgaaatttgaatgtagacgagtgttgatttgactgctacagtttattttacgtatagaaataaaataatgtgtagcaaaagcattattacaaagatgataatgaaataactagtatttataatagtataatagtatagtatttataatagtatgatagtttaatgactatttgtcagatgttgtgtaagaaactttatacacacacacacacacacctcatttaattcctgtatcaatcaggatacaggacgctgtggtaacaactcctcaaatctcggtggcttgcacaacaaatgcttatttcttttttttttttgacaccaagtcttgctctgtaacaggctggagtgcaatggtgcaatctcggctcactgcagcctctgcctcctgggttcaagcgattctcctgcctcagtctctcgagtagctgggaacacaggcacgcgccaccacatctggctaatttttgtgattttagtagagatgggatttcaccatgttgctcaggctggccttgaactcctgacctcaagcgatccacccacctcagcctcccaaagtgctgggattacaggcatgagccactgcgcccagccccaaatgtttatttcttgctcatgtgacatgtacttcctcgagtttttccttcctgagatctaagctgaaggaacagctctctggagccacgccattctggtggcggaaaggaagagtaaaagtggtagaaccttgcaatgctcttgaagcgcctatttggaatgtctacatcatgtaaatggtaatggacaagtatgtataatccccacaccaaaaaaaggggacactattggggacaataaccacatttcaatgctgcaagacggatattgactgcacccccttcccactttcagaaagaagaagagtaattttgctgaactccttctagagactggaaatgtcccttccagttggggtgattagggaaggctttggtaaaatttgagctagagtttgaaggttaggtagactactggtgggtgaagaaagaacaaggacctttgtaggcaaaggaaaacctcagaattacagaggtggaaaaagagttctagtcaagccacttcagctggctacagagtaggtgggaaagaaaatgggaggacaagggctcagatgatggggggttggggcattggggggacacttgaaagctaaactaaggggttgaacttaatttaggaggcagttagaagcttttacatatttttgagcaagagagtgacataattaaaatgatctgggccaggtgtggtggctcacacctgtaatcccagcactttgggaggctgaggagcttgggtcacctgaggtcaggagatcgagaccagcctggccaacatggtgaaatcccgtcctactaaaaatacaaaaattagccgggagtggtggcatatgcctgtaatcccagtagctgggaggctgagacaggaaaatcgcttgaacccgggaaacaggttgcagtgagccgagatcgtgccactgcactccagcctgggcaacagagcgagactccatctcaaaaaaacaaaacaaacacacacaaaaaaccaaaaataaataaataaaatgatcacttctgaatactgatctaactaggggttgcagggtgggctgatatagggagaaactggagagcaaggagatcactaaggtccctacatgtccagaaccaagatagaggtcttgaactaggatggtggcagttagaacaacaacaacaaaaagtcaattccaggctgagtgcagtggctcatgcctgtaatcccaacgctttgggaggctgaggtgggagttagaaagcagcctgggcaacactgcaagacctcctctctaaaaaaaaaaaaaaaaaaaagttagccaggtgtggtggtgcccacctgtagtcccagcaactcagaaggctgaggtgggaagattgcttgagccccaggagttcaagcttgccgtgagctacgattgtgccactgcactccagcctgagcaagaccttgtctccaaaaaaaggtcaattccactgacttttctaaggtgtacaccatcaaggggcagctccatctccaggccattggctcatgagacattctgtagtcagaaggctagggcagattgctttgagcaagcccccatggtggttctcactcctacttctttgggtatatgcccctctgtttaaaaataaagttaatatgcatttaaaaaaaaaaaggagaaaaaggtcagttccagaaactgtgtgaataaagcattttacttgctttttctattaatctataacatatgttgattttttaaaaagaatataagagctatgcaaattggagcttcaagacaacttcccatctccctaggaggagatggctgccctaaacccccctacatagaaatcatcccactgcttgggcttaaacttgatgttggggaaatgaaaaatccaagctaaggccgaagcctggggcctgggcgaccagcagaatgaggaccactggtcagtttcaggctgaggtgcgtcttccaggggacaatctctagctggcccttaaacattcagacttcaagctctatttacagcataaaggtgtttcaaaagacgtgatacaaataactgcaaatgctctgcgatgtgttaagcactgtttgaaattcgtctaatttaagatttttttttctgacgtaacggttagattcacgtttctttttttttaagtacagttctactgtattgtaactgagttagcttgctttaagccgatttgttaaggaaaggattcaccttggtcagtaacaaaaaaggtgggaaaaaagcaaggagaaaggaagcagcctgggggaaagagaccttagccaggggggcggtttcgggactacgaagggtcggggcggacggactcgagggccggccacgtggaaggccgctcaggacttctgtaggagaggacaccgccccaggctgactgaaagtaaagggcagcggacccagcggcggagccactggccttgccccgaccccgcatggcccgaaggaggacacccacccccgcaacgacacaaagactccaactacaggaggtggagaaagcgcgtgcgccacggaacgcgcgtgcgcgctgcggtcagcgccgcggcctgaggcgtagcgggagggggaccgcgaaagggcagcgccgagaggaacgagccgggagacgccggacggccgagcggcagggcgctcgcgcgcgcccactagtggccggaggagaaggctcccgcggaggccgcgctgcccgccccctcccctggggaggctcgcgttcccgctgctcgcgcctgcgccgcccgccggcctcaggaacgcgccctcttcgccggcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtaagcgccgcggctccgaaatctgcctcgccgtccgcctctgtgcacccctgcgccgccgcccctcgccctccctctccgcagactggggcttcgtgcgccgggcatcggtcggggccaccgcagggcccctccctgcctcccctgctcgggggctggggccagggcggcctggaaagggacctgagcaagggatgcacgcacgcgtgagtgcgcgcgtgtgtgtgtgctggagggtcttcaccaccagattcgcgcagaccccaggtggaggctgtgccggcagggtggggcgcggcggcggtgacttgggggagggggctgcccttcactctcgactgcagccttttgccgcaatgggcgtgtgtgtgtgtgtgtgtgtgtgtgtgtgtgtgtgtgtgtgtggaggggtccgataacgacccccgaaaccgaatctgaaatccgctgtccctgccgctgttcgccatcagctctaagaaagacgtggatcgggttctagaaaagatgactccctgcacgcccctccctgcacctcccgagcagtgattccgacagggccttcactgcccctgattttaggcgggggccggccccctccccttttcctccttcagaaacccgtaggggacatttgggggctgggagaaatcgaggagatggggaggggtccacgcgctgtcactttagttgcccttccccctgcgcacgcctggcacagagacgcgagcagcgccgtgcctgagaacagtgcgcggatcccactgtgcacgctcgcaaaggcagggttcacctggcctggcgatgtggacggactcggcggccgctggtccccgttcgcgggcacgcacagccgcagccacgcacggatgggcgcggggctgcaggtgcatctcggggcggatttctttctcagcgctcggagcgcagggcgcccggcgtgtgcgctccctgccggaggcgcggggctggcgcgcagggctcgcccctcactgcggcagtgggtgtggaccctggtgggcgaggaagggggaggataggctgtgcctcctcccactcccgcccccagcccccctttttttccccctcggaacgcgaggtgccatcttttttcggcgtgtcacgtctttacggtgccatgccaaaccgggtggccgggcttcataggacagggcggggcctggcattaaagggagggggacaatcagcgctgaaatcttggcgttttgctgctgcgggcgtgagcactgggggcgttcgcccagcaccttcttcgggggctctttgctttgtctgtagaggttacgtgatctgcgctcccagccctggtttctggcttttattctgagggtgttcagtcaacctcccccctacgcccatgcgcctctctttcctttttcgctcctcatttccgagcccattgttggatctcgaggcttgctgggttcgatgaactcgagtcaaccccccgacccccggcacgcatggaacgggcgtgaccgcgcgcagcctcgtctcggagtctgccggcgccgggaagcttctgaagggatgggattcgagtctccgtgcgcgctgcgggcggcggcagagggatctcgcccctccctacaccccaagtgtcctgagggccacgccacaccaggttgcccagcgagggacgctggctacccatccggggatgggtggggagccctggcggggcctctccggctttacgccctgttgcttcgcctggccggagaatgtgaggaaggggcataaggttactggtgcttcggccacacccatctttctgagcccactggactgggcgcagaggggggattgccatggaaaccacaggtgtccggagaggggatcttggggctggcctcaccccttccctgcggagattggggaccctggggtagggggagccgcgcccagtcggcctcctggaggacacgggaggaagccccgaacccccgcgcctgaggctgtttctgattggcccctggaggccgcagacacgcagataggcggccctgggtgtatttttattaatattatgtccgtactgattaatattatttatcttaaataaatttcacccgtgtccaagttcaccgcgcccccaaaaccgagtctggggcggcagggggaactcctggccaacgaatccatgcctcgccctcctgtgatgaacctggtacgcacggttttctggttaattctatcgctgaaaactggtgcggggggcgcacttctgagacggaagagcatctaggagctgaatcctccacgcgggtcgcccaggttgatctgaatttctggggaatggcttggctgcccgcccgggaccaggccgaccctccttgacggtggcgtagagggctggagcctgggtactgcgaggctcctcgcatggctgggcccgccgcgaggggttgcagagcggctcagggatcgattcaagcatcgtctctcctccctcgcccccagacagagctgggcgcggggttccccttccagatggagcgagggtctcggggtggccccggaaaaggggagcccgcggccacggctacgtattgccatctcgcgagcagagatgtcacctcctgcctttggaggaaagggagcccggtggggatgagcgcatttagcccaatgctgggaacaaagcgcactccgcgcttctgcgatttcgctccattttgaaatgtgttggcgctttggtggggccgctgcggtgggcaaggccgggggcgctgttaatggaggaacctcagggggacggtccttcgtaggaaactctatcctggctctgcgcgcgctttaaggaaatggcttccctccaggacctcgagggatgcagcttttgcgcggatgacggtggggtgctgaaccagccggtgcgcctctggaaatgtctgggcacggatcctggggccatcgacgactcctccccattcccagcaggcgggagctcttacattccgagcgagtgacccctctcaccctctggcgctcacacacctgtaactccaaacctccgtctcagaatggtccaggctggaagggatgatgggggctccgacagcgactgcctagctcacccctctgcgtgctcaggctccaggctcagcaggaccaatttgagttctatctgatccccctcggccccttaactgacccatcctacaggagacagggaaatgtctttcctaccgcggttgattctggggtgtcattttgtgttttgtgatggctgcttatatttactgtataagcattgtatttactgtataagcattgtattataattactgtataagctgcttatatttactgtataagcatctccaaatcctccctctacgtaaacaaattaatggataaacagataagtgtatcccctgcccccacccctgctacgcaggtccggagtgactcttgaagctcatacattccttggccaagtttgcttctctaacagatgtttatatagcaataacctggcttggctcttgggttcacctttggacgatttggggaaggggcttgttggctttgctgggttttggatgagtgacagtccatgactgttcctgctggaagggcgtgacttttaagtggtttctaatatcaggcattgctcctccgacaggaacaaaagaaatggatactgcccataaattgttagaaaacttagaatcgctttgattgaggaaaggttagatttattccggttggaaaaagtggcctttctattaaacgtgccctttgaccctcatgcccttggaggtcggtgccagcctggagatgggataagattgtggttttccttctgcctttttaacatctgttgttacagtccatttgttgaaaatttaaagaaactgttttattccactttccctcagcatttatgtgtgtggtttcagtagctctgtggctatatgtacgaacacgtgttatttttccaattggacatgtgataattttccaactggaccttgccttctattgatgtatttatttagcatcttccttactccctccttgaaaaagaatcactcaaaaacaaataaaaacagccgtaggggcctaatacagtgctagacatacaagaggtattcggtccataccaaatggattttatccatgaaggataaatggggaaatacagtgggaagcaggtgggaaactgcgtttgactctgctctttcctccaccaccactttcctcatcaccgtgttcagagacccccaaagccccctcacactcccagaaacacccccctggccactcctaacttgccatgcccaggagttaggtgcttccactagtgacatggagctggcgtttggggggcacctcagcaggtgacgggaagagaagaccccagcctcaccagctgggctgcagcagggagaggagtcctcatgttccagcagggactctcagctgttttcctgtaaaaccatggttctcaactgggggccactgagatgtctagagagatgtttttgttttcacaactcggggagggtgctactgacatcttgtgggtagaggccaggaatgctgttaaacatcctacaaggaaggcacaggacagtctcctacatcaaaatatgacccagtcccaatgtcaccactgctggggttgacactggcactgctatcttaattacattcattgagtgtcttttaggaggccctattctaagtgcttgctaagattatctcatttaatcctcacaacacttccgctatgtagcaggtgctgttattatctccgtgatggggaaactgaagcacagagagggttagtaacttgctaaaggtcacagagccagtgggtggtggagctggttgcctgacactagttccctcccctctcagccacatgtgggtttacttggccattgtggactagtctgggaacccagatatgatctataacattgacccagtagaatattgattccaaaaccactgtctcacaaatgaatttttacaagagtctgtaatcggagcatgacccagaataaggttagggagatgtggagttaaagctctcaatttcttatctggccccgacacagagagcaaggcatttcactctacattggtgctctgtttataaaacaaagagcaaatatctcttcctaaggtccttaaacctcttcccccaatccagggtttctggactgctctgccatatgacggggcagctggtttgattgacccagggaaggctggaaatcaagactgggggatcaagacgtagattcagtgtggccaaggtcaagtctctgaggtttagggacatcagatccccagcttaggttctgtacctcggcaaggtgaaagcgttggcgcccactgatgaggcctgctctgagattgtgggtgtgggttgagttgggtgggcataggcaagtcctcttgtaagaatcttttggcaaagatgggcctgggaggcttttctcacttcctggggcccaggctttgcaataagtattccattatactgtggtaccttggggctacctgagaatcctctgtctcgcccctgttgccttgccaaagagtttgctgtccaagaattcctttcctgtctccaggtgccatgctcctgccacctctgccaggttccctgcctgcccagatggctcccaactgagtgtgaggaggaatttgagacaggttttgagctttctgggttctccagttaggaaactttctgtaagcatgcagatagaatgggcttcagcaaaatacaaactcgaacaacttccatgtatagtcccttaattttctttgcttttttcatatttcatcaggctccatgctgagcccaatcagggacccgatagaaatccaaacaccatgtcagcgagtccccaagaaatgcattttgtgccaaggctattcaaggaaggtttgggagcagctcaagggcagacactgttaccctcccccaggtccccagtgcagggcagtgttctgcatgtggaggcagtttggcctaatggttaaggaggtaggctctgatcgggcctcctgggcacaaatcccagctccctgctcactgtgagacctaagccatattgtttagctgcttggagagttttttgtcatccacaacttggagtatgatggtacctgtctcacgggttgccatggggttcacacaagctaacccggtactcactagggccaagcacatagtaactgctcagtaaatggcatcatcggcggtgtcctgtggatgagtgcttgtgattggctgaatgaccagaggggtctaaagatcctggtgatggaatcagttgtacagataaattgttacactgagtagggatcaagataggaaaagtcggcaactacccagctcccctgcaccaaactgggcagaagtggatcctctgaaaattgcacacacccatgtttaaatgtacacacagaactcttgccacaggcaagcggagatttgtcatctgctgtccctgcctcatcttcttcctgaaatccactccatgccaggaataaactgcatgctctccaccagcccaaactgacctgccttcccgccagccatcccgggcagggtgacctggcttagtacatcgggttcagagatctttccagtttactcgttgaataaaaagtgagggctgatcgagaaagtaatggcagtcagggaaggcgaaggaggtaaagaagagattttacaaatgaagtaattcaacagagtgctgacattggtaaactggcaaacagatttcagggtggttggttgagagtagagtagaaaaggattaaataaagcaaacttgtggtgtactgaatcttaggaattccatgtatccaataagtatagtcatttatgaattaataaattcggcctaagaagccttcttatcgcttaaatcaagactaagtaacaatatatcagttttaaaaagtcattatatcagaaaatcatttaaatgatacacatagatttccaagattttactttaaccgaaactatataaatgtgaatttgttcacccatcttttgacacagggctcaggtcttctcttggtgtctggatcagccagttgaaatttcttgtctgttttgcctatgccacattaataatgcactgtctgggtcctccgatttcagtttggattttgggtttacattgtggagtcatctgaatgcagaatccttcagggattttacttttttttttttttttcatggtctttaccatcccatttgatagtaaatattactcacctttatgaagtctttccaaaacattcaactaaattttcttaaaatcattgaatgatttgaagagcttattcctcagcacttttactccatcagcttgcaccttattttttaatctttttttgagacggagtctcgctctatcgcccaggcttaagtgcaatggcgcgatcttggctcactgcgacctccacctcctgggttcaagcaattccgcctcagcctccgccgtagccgggactacaggtacacaccataatgctcggctgatttttgtatttttgtagggatggggtatcgccatgttggccaggctggtcccgaacttctgacccaagtgatccacccacctcggcctcccaaagtgctgggattacaggtgtgagccaccgcgcccggccagcttgcaccttatttaggatatgtgattattatagcaagtctggtgtacatacaagattttgaatgggcacagatgacctttagtaagtgcttggctgtgataagaggcagtcctgactgcagatcaggctgtgtggaccccagccttgcatgtttacagaccttcatgtcttattcttacagggtatcagaagaacacctactggggaaacttataaattagtaaaaggtgggcattctccccgcccatcttctgtctgtctgccaggactagcacagcactttgaagtcattcacatagaatcccaacttaagagggtaaaatcctcctcaacagactgaaaataagtttaaattccctttgctatattaactcccctgaggaaagagtcttagatcaatgtccaacactaaaaacagttttaaatcagcaagtgagaattaaatctgaagcaattgataataatgtttcattcattcctctcctttggccccgtccaccctactgctaaatccaggcatcaaagagaagagggacataattatctctagtcccagctgctggttttccttccagcctatggcccagttttctgttttactgagaaggctggtgatgttatcttgggatctaagtctgcagtttcaccacaaaaagtccagggatgcactttcatgcttgtgtcctcctccctgggatagcaaggatattagaagacccctggctctgtaattgcttgtcatgtgctctacagacgccacagaatgccaagaacgaagtgctgggaaggacaaattcatggaaccgtgggacggtgctcctcccccagcgtaaaggacagctcctcctcctgaattggagccagcgttctaaatcatgtgtcaacagagttgtcctggatcggatccagttctgccattgatttgcaggtcatttcagtggtacctgtttccagttgttcttaattgaacagtggcaccaaactattgtcttgcctcatccccctcccatggcctgtcccccaaaaagagacttcttgggtaattaatcagggcaacatcaggcagtctgggcgcggtggctcacgcctgtaatcccagcactttgggaggccgaggcgggcagatcatgaggttaggagattgagaccatcctggctttgtgaaaccccgtctctactaaaaatacaaaaaattagccgggcgtggtggcgggcgcctgtagtcccagctactcgagaggctgaggcaggggaatggcgtgaacccgggaggtggaggttgcagtgagccgagatcgcaccactgcactctagcctgggcgacagagctagacttcttctcaaaaaaaaaaaaaaaaaggaatctctttggttttatatatattttttttatatatataatatatattaaaatataatatatatatttatataatataatatataaatatattatatattatatattttatatattatatattatatatattatatattatatatttatatatttatatattatatatatttatatattatatatttatatatattatatatttatatataatatatattatatattatatattatatattatatattatatatttatatatattatatattatatatattatatattatatatttatatattatatatttatatatattatatattatatattatatatttatatattatatatttatatattatatatatttatatatattatatattatatattatatatgtatatattatatatgttatatattatatatatttatatatataatatattgtatatattatatatctaatatattatatatattatatatattatatattataatatatattatatattatatattatatatatttttatatatataatatgtataatatataatatatataaaaacatatataatatatattatatattatatatatattatatatattatatatattaaatatattttatatatattatatatatatacacatatatatatataaatgaggccaggctcggtggctcacacttgtaatcccagcactgtgggaggatcacttgaagccaggagtctgagactagcctgggcaacaaaacaagatcctgtctctacaaaaggaaactgtaaaaattagctgggcatgatggcatgtgtctgtagccctagctacttgggaggccgaagcaggaggatcgcttgagcccaggagttcaaggctacagtgagctatgattgtcccatagcactccagcctgggtaacacagcaaggccctgtctctaaactttttttttttaattctatttatatttacatgtatttaaatgtgaatattcactacctatttgttgcatgcctgcattttttatactgggcttgccaaaaacccgaacagctttctactttgacaatgtatcagaatttaaatcagcaatatgttaataagccaagcaaaggttatatatgcaaataaaactgttgtctataacctcctgttacactggggcacagcaaaagtcatggtgtagtcgcatgtgaacctgtccctttcatagctgctcattgccaggaaacatcaggaatagccatttggaagagtcatcagccctcccaccatccgttttctgtcttgtcttttccctatgagcaggggaaattccacgctggccccaatccccagtgcagcggctcagcctctgcctctgctgctggtccccatgaggccagcttagaaacggaggattttgcagaacatccctaaatccgcttgaataatgaagtgatcattcataaactcacctgaaccttattaaaacctatttaatatttttcctggataatcctatagggataacttgcctcctgggcttctctccaccgggttcagttcttcctttagtggtgaagttcctcccttcttagcatctcaactgtgcctgagaaaaggccagtggcggctgcactctgttccctgtggagtgttaataaagactgaataaattgaaataaatccctttcaatgtcattaagtgctataaataatcatgaaccaatgttcgatggctgatgagaaatgcaagaaaaaatttttaatcagtaggattcataagttgacaatctgggccaagttaaaaaaaataaaaataaaaagacttttaaaaagatcttatcgtttgttaccagtaagactgaattccagaagcaagctactccctcatttgtgggcccctgttatcactggctgcttagggttgccaagccctgaattcatttgtcaactaagagatttttggccaagattaagatttcccatgcctccatatttccatctgagaaatggagattatactgtcttccccctcagaatggatgataatgtggtctctcttctgttcgcatagtcatagaactgaaataaaacaacttaagagaattcctttgagcttctcagaagtgctgcagggctgggggatgcctcccaggagccgcagtcaggtgctgatctgaagtctttggtgggctgactttagcctgacctgaaatagtatagctgctgccacctggctcccttagcgtcagtcagacggtgcagctggttcctaggggtgagggctgagccagcagggtccgtgcccaggagggatgcatgggtggccacagcccagcctgcactgatcttgtctgtccccttctttggaaggaaggagccccaaaccagggtgcaagacagtgggtgggggtgccttgagcatgacctcaagtgatttccagcccctgccagtgctgacttctctggggaagggctgggacttccttctgggctcaagtcacgacccttggatggaatttcctgggagcttttctgttttttctggagttttcagttttttcctaaccagacagggacttggtacagaatctcatattctaattatgcctaggagcagcctctccccaccactcacagtgtttagcatgtgacaggaatcgattaaggcatgagtgattaaattaaagccaggcattgacttggatggtgtaatattctgacatctgtttggtgtcaaaggcacggggcaggcgcgttaattgaactgcttgcacctggcatttgaattgagccagagcggggctaaagtcagtttgccttcaccctgtaaatggagggtttctccggagcgtggatggtgggaggtatttcagggtgtatgcataacccccaccctgacaatggcccatctcttctccagcgtggccaggtttgagtgccagtcctgggtgtccagtggccccatagccttgcgttttagtaaaatgctgcccccattaccacctggtctgtgcacttcggtcactggaatttgccatcttccagtcccgaatgtggcaagccatggagccttaagctcttctccctccacatcctggaacagacccgccagtttcttccaggcattgcctcagtttgcccctctgtttccagtcacactctcaccagcgataaaatgattttagaccttatcatctcaccctcggatccttatggaaacaataatgagttgttccctgtttcaattccaaaattcatatccaatccgttttgcatgccattgccaaattcctcccagagcaaccccgtcacctgccctggccctctccaagtgtggtcctgccatgggcatcgcctgctaagccaagctggcctcgagctgcctgcccgggtccccacaccttggctcacctccctgcccagtcccgcctcctgccagcctgccctgtggctccttcatagatgccgtgctctttctgccccttgctcacccatggcagccttgcccctctctccctgccccaccccctatttaaattgacctgaccttcctcagtgtccatcttccccgaagctttccccagccttggcactcaaggtccagaggctacgcgtttcctctcacctgtggcagcgccgtgctccccagtgcctcacagtttccttcttgcccccgcttcctgtgtaggactcatctgcccacaggttgcacgtcctgtgagggcaaggactgtgtcttatgtgactttccttctccagtcacagagctgggcacatagatagctcaaaaccctctttattaacacagttggatgttgagaaatcaaacaggccaatgtcaaatgagctctccttatttaaatcaagtcagttctccacctcctagcactcagttccagtactctatatacatggaaataataaaaaacacatttcctttgaaacattctataatcgttcctttgccctacttcagaccaacttaacgcactccccattggtccaaatgagttttgctatacgaagatgctgataataatagcagcagtggattattctgctaaaaccattgcctcgttaatcctcagtcccgaggtggggattattatcctcattttgcagagaagcaaactgagactcagagatttcacagctggggagggagccagctcatccctctgtccaggcccaagctctctcccgcttgccttcctgcctctgcaacctcagagcatcccccatctggttctactgcctgtgctagtcgtgcaggagccaaaagacacgtctttagtgctaaggactggagaagccatgccctccagcctctgtgaatgggtcatatgtaacatgagcctggagaaattatttgaaaccaaaggcaagcctctaaaccaggctgctgcttcatggcgccggtgacggcagaaccaaatttagtgctgtgggcaggtccacacttatcaaatagagaagctcatttttcttccggctcacatcaagcatgaaaaatgttcacacataccccccacacacacatgctttccggaggggtccatgtggctagaggctggaagatgtggatgagaggagcctggcaggtaagcccagggaagatgacattcagcttcccagacagcatctacagggagaaatttaattaaaagtggggcggtttccctgagcaaggcagacaaagtcagccctctactgttaagaaaaagggtcacagtgagaggggaggtgaggagactgagtctgtattttctagtctgttgggctacactacctgatcccccttcctcaaaaatccactttactttccccatgtctacaccaatgtggttcacactctgggaccaggaaaagggggagtgatggggaacagagaagggaggagctcacacagctgaggctggggttatgcatatcgaattacttagaatttgcaacctcacagggtactttcatggcgttgaaatacacttcccacagccaccctccctctaactaaaagcaagagtcatttctcagttctggtcttgcctcccacgttctcctccacatttaagaaaatccaccagctacaaagtgaagataccatatgtgatatcccaccctagtttctgttttatcagggtttggagcaggtggagcaggcagagggatcatttcagcctataaattgtattaagggtgagtactgagtcattcttcaagaaaagttttagaagcatccaaaactgaagggtggagccacctggagacagtatcatcagtcctggccccgagcatggcctgcataggcccccatggatcccagcgggagctgcagagtgcgggcaccttggcacacagccctgagtgcaaaattaggagctgggcagagggcatctctctgtcgccattgggcagcccagggcacactggtcatagccttagaccacgaacaccctgtgcccgggggacagatgcaaccagtgtgccctgggctgcccaatggcaacagagagatcgacacctggaccccatgtcacggggactccactactaaggctcctaagactgccaccttccagtgggataagccctgcctcctactgggcccacaatgtgcagagaacacttgggactacctggctttctggatacacaaatattgatccaatctggactaattagaaggtcagtcccaataacaaatcgaagtcagctgggcgtgatggctcactcctataatcccagcactttgggaggctgaggtgggcagatcatttgaagccagaagttcaagaccagcctgggcaacatagcaaaaccctgtctctactaaaaatacaaataattaggctgggtgtggtggctcatgcctgtaatcccaacagtttgggaggctgaggcaggtggtcacctgaggtcaggagtttgagaccagcctggccaacagggtgaaaccccgtgtctactaaaaacataaaaattagccaagcatgatggcatgtgcctataatcctggctactagggaggctgagacaggagagaatcgcttgaatccaggaggtggttgcagtgagctgagatggtgccactgcactccagcctggttgacagagcaagactctgtctcaaaaaaaaaaaaaaaaaaaaaaaagccatgcctggtggagcactacgtgtaatctcagctatttgggaggctgaggcacgagaatcacttgaacctgggaggcagtggttgcagtgagctgagatcgcgccactgcactccagcctgggcgacagagtgagtgagactccatttcaaaaaaataataaatctgagtcactttaatattgttatttggatgtcaacctctaggtgtttgagacaggagagtgatatgggggcactggaaacacacaggcacggggtgtcctcacacttgggtagcccacacgatgtgatttcagggtgctgggaggtccccccactccccaaattactaacaagtggatagtactttacagtttatatgatctcatttgattcttaacatgagcctgtgagtgaaaaattccttcccctcttctacagattaggacgttgagattcagggaggttcagagggattcagggaagtcaagtggcacctggagtcccgtggctaatttgaggccggtaggggattcgaacccaggatttgtgcttcttatgcctgggcttctgctccctggggcatggtcttccccctagctttcccattcactgctttagcctaggggtcctaccctttattaaactgccagtgcctcactgcttttctcccccaaagacaaaaaaaaagtgtttttgcttttgttttgtttttcatgggcagagacctggaatttcagcttgagaatttgtgccatatgataaataaatcaacagatggctttttccttaaaaaaaaaaaaaaaaaaaactaagatgtatttgcagtgaggcataatttgtaccaaaaagtgctcaccacactgtagtcatgggggcaggaggcagccgcgggtgaagggagaaatcttggagtccaggcagcccccttctgggctgaactggggagctgggggtgctgccagccctgccaggttctcctaggaggcggcagctcatatggctgtgggaggaggcagagggagcctcatatgcacccacatttccagggatctagaagacagaaggaggaaaaccaccatcatgttaaagcagacagttaggtaacacatcctgtaatacaagttattttttccacatctaaaggctaaaaatagttgttagaatttaaagataattggtaaatgagtttctatccttctagtttcacatcaaatggaatcatgctgccttcacatcactagtgcccgttatttgtgtttaatttccacaatgttgtctaattccactctttgggcttccccagggatccagcctccctcactcgcccatcgcagggagatgctttattcatctttgtgtcttctgtgccgggcatagcgcatggcacagaataagcactcagtaattgattcacgagtgaataaatggatgagtgggtgagttcaatattgactacaaaaaccctaaggccacactggtgagtggctgcgcctgtagtcccagctgctggggaatctgaggcaggaggatctcttgagcccaggagtttgaaactagcctgggcgatatagcgagaacctgtctcaaatgacaaaaacagggccaggtgcagtggctcacgcctggaatcccagcactttaggaggccaagatgggaggatcacttgaggccaggagtccgagaccagcctgggcaacatagggagaccctgtctctacaaaaaattttttaaaaattagctgggcatggcggtgtgcgcttgtagtcccagctactcaggaggctgaggcaggaggatcacttgagcccaggaaattgaggctgcagcgagccatgatggcaccactgcactgcagcctgggcgtcagaacgagacctgctctcaaaaaaacaaacaaacaacaaaaaaaaaggctttcttaaagagacttgagaacagaaaggggaacagatacataacttatatatttatttgttcatctttccaccttcctggagggtggaggggaacaggtctgtatttggagttttgaatgctaaaagtgggaatacatgtactgtttgccatgatctgttcaaaagttaagccaaatgccttagattctcctgaaaactggaatgccactgtaaactataagccccacttcaaagataaaagatcttgatgaacagggctgggtctgtggactgggcctctccccaccacacaaggaagggtggtgccagttgaaggaaaatcacttaaatccttgctgtctcctaataaggtgtggtcccaggtagggctgtcagaattagcaaattaaaacacagggcatctgtgaaaattagaatttcagataacaacaaataattggcataggctgcataatgtccctcaaagatatcaggtcctaatctccagaacctgtaaatgtgatcttatttggaaaaggggtctttgtagatgtggttaaattaaggattttgagatggggggattatcctgtattatctaggtaggtcctaaatgcagtcacactcatccttgtaagaggaaggaagagagagatggaaaacacagaagagaagacaatgtggtgatggaggcagagattggagtgaggtggccacaagccaaggactgctggcagctaccagcagccagaaaagtccaggaaccaattctctcttggagctccagagggagtgtggccctgctgacaccttagcttcaacctagtgatcctgattttggactttggccttcagaagtgtgagggaatgaatatctgttgttttaagccaccaagtttatggtcatttcctacagcagccacaggaatcaaaaacagtaagtatgtcccatgcaatgtttgtgacacacaccaaaaatattacttgttgttcacctgaaattcaaatttaactgggtctcctgtattttatttggccaacctagttcccaggcccaaagaaagaggcttttgaaatttgcaagaaagctggttggagctgtcagaaagtggactttgtaaacacagtaccaccgaaccaatttgaactgtactacctctagacaaaagagagggcagtcagacagttgttcgtgatttcttctttcaacagtcatttgagcacttactacaaaacagaagctatgtgtaagggtggaggcgttagctgttaatcaggacctccaggctaagtttctgtattagtccgttttcacgctgctgataaagacatacccgagactggggaatttacaaaagaaagaggtttaattggacttacagttccaagtggctggggaagcctcacaatcatggcagaaggcaaggaggagcaagccacatcttacatggatggcagcagacagacagggagagagagcttgtgcaggggaactcctctttttaaaaccatcagatctcgttagacttattcactatcaagagaacagcacagaaaagacctgcccccatgattcagttacttcccaccagatccctcccacaacatgtgggaattcaagatgagatttgttaccatatcagttaccaacccttccagataaatcacgtgaaatatcgccattaacagagtgagctcaggtggttcttcagtgcatttctgatacctgaaccttccctgggaatttcacagaccatcaggctctccaccctttgatagcaggatagcagggcccaggttctgcaggaggagatgttaccacaggcctgaaagggagggaggggcagatgctacaggaagatgctggctctggattcgctggaggagctttcaagggaagtagatacacactgtctccatcatttcatgtccatcacactctaaaatgctttggacaagaagcaaatgttaaagacaaatgtggcccattttcctgtacaaagagggctgctcccatgccaggctattggcactggtgggcatgaggcttctctgctgccctggccggggggttctctcactcaccattggctctctgacacctggagagaccaccacccttgggctttcatgatgctcacagaatccacactgttggagctttaaggagcctggatcaactggaacaggcagggagtactaggacagcccagcattgccccaaaatatccaggcctgataaaagagaaaaacaggtagctcacaggaaaaggataaaaaaaggaggagggatttaacatgaaaaggtgcttgatctccctcataataaaaagactgctgattccatccaggcaagtgacagaaaaaaaaaaattaatttaaaaagactgctgataaaaccacagcgagacactgctgctcagggatctgagggtgtgggcagccaggctgccacgcatcatgggtcggagaggaagaccacacccctggagcagagggcggctgatctgtcagatgccctttgacagcacctcagcttccaagaattaaccctttctatgtgagcagaggcatccatggggggacacactggtgaatcatctgttatgtagaagtctggaaaacatcaggatggaactggtgaaataagtgtggcctctgacggaatggagcggtccgtctgcactgctgcgggtgcccctcagatcctgtgggtcagtgagaaaagcagtgaggaacaaggcaggtactgtgtactgtcctctgcgtgcaaggaaggccagcgcatgcaacagagtccacacagacatagcctaactctggaaggaagaatgagaatgcagtttcagtggtggcctctggtggggagaaactgggtgaagggagatgtcatttccatttctctactattaattttgtattaccatgcttaaatgttactttttacctttttttttttttttgagacagggtctctctctgttgcccaggcaggagtgcagtggtacaatcatggttcactgcagcctgaacctcccaggctcaagcaatcctcccacctcagcctcctgagtagctgggactataggcacgcataccaccgtgcccagctattttttttaatcaagatggagtttttctatgttgcccaggctggtctcaagctcctggactcaagcaatcctcctgcctcagcctcccaaagggctgagattaaaacgtgagtcaccctgcccagccaattgctttttaaaaaagattaaatgcatgtatacgctcaggcatcagcacacttggaaaggatgaaaatatccggaagaagggttcttttaaaaggctcctcaagtgatgctggcaggcatgacgaatgtccctggtcacaaaagctctgatctggcctaaccctgtcatgttagagactggagtgcgtgtgtgtgcgcgcaaagtgtggggggatgggggtgagtgtgtgtggtgtgtaagcatgagtgtgtatgtgtgtggtgtgggggtgtgtgctgtgtgagcgtgtgtgagtctgtgtgtgtagtgtgtgtgtgaagtatgtggtgtgtatgtgtgacgtgaggtgtgtgtggtgtgtgagttgtgtatggtgtgtgcatgagcatgtgtgtgggcatgtgatgtgtgtgtggtgtgtaagcatgtgtgagtgtgtatgtttgagcatgtgtggtgtgttgtgatatgtgtgtggtgtgtgagcatgtgtgtgtgatgtgtctgtgtgtggtgtgtgtgagcatgtgtgttgtgtgtgtggtgcatgtgtgtggcgtgtgagcgtgtgtgtgcattgtgtctgtgagcatgtgtgagtgtgtgtgtgttcagcatatataaggcatgtaactgaacacagcactttagagggctctcctggagtcagagggggtgggtaggaggagaagggaggtgggctagtgtgctgaagtatctactccttgtcatagtctgtgacaacccagactagcccatgagccaccctgttccctgcatttccaatgagacctcggtggacatgttccctgaggtgaggctgactgatgtcatttgacgatcttgatgccaaatccttttatatcaaaaacaaccagaacactctcttttctcttagtgctttcacccagatgaccacatttcatcctcccagccactctgggccaggtggcactgctggtttgaaagggaggtctcccctggagtaacttccgtgggcggattcacaccctgcccacagtcctgtcccagtcagcccaccatggtggtctccggttcctccagaattcccgcttttcagctcatccccacattcccggagggactgagagcgcagccccagggccctgctctttgggggccgtctctacacccagagaagcagcaaggcattcctaggtttctctttcagatgcagaacttcagtgttcagagatgttcccactggtcctgagagggctcagttcagctttaatgactgcgctgttgcgtgtgctctgcagagggcgggtggcccagcgtggctgactgcagttttcctgacgtggagcccgagcctgccccgctgtttattaattaaggatcactctgcttgcagaaccctgaactccccagaactgtgaggtgggagaaccccgagaggccacctggccccacttcccacctgctgcccaaaccccctctctgccttcctgacagtcaccccaactcccagtgatccccatcaaccatctgacaaggggactgagagggaagagaaaggaggggcccaaagaggaaggtaaaactgtcgggaacagcccccaaatgtgtgacagccttcagtggagttgcccactttcccttttctcctccctgcaggacctcccttctccccagtcctccccaacttctgaggttacattgagaaaagtctgcagagaggtgccagcatcacaaggtgttaaggaccacgagtttggcattttaacagatgccagagccacttgagaaatgtggtaactaagcccagagaggtacagttaacctccccagagtcacacagcaggttcatggcaaagctggactagcacaggtgtccttcccctgcagatccccttctgtgccccacatcacctccctccagtgtctgggccacctggagatgggccctcagactcacccggccagaggtgccatctcatgggagaggtctggccaggaagcatcgatatttgagatcccaagaaatgaagacttggcctgtcagatgacagacttcggtcatgggaacacgtgatctgttttacacatgcgtcccctcagcagcagctttccagaacattcccactttcttctgtagtgagaagaactctttccctgcagcctcctgcccaactcctccttcagtgtctttgcttcagtgtctttgataaaccattctgctttgcagagtgcgagctctgccttgcagggttcgcatctgcctgtgctgagtaaccaacgctaaggtcgagtggtcggtcacctctcataagagctagggttgtctcatgctgatgactaggacttgccctcaaggagaaaaataaatcaaaacaaaagcaaaaacagcaaacatgcatctcttaaagaaggctctgagtccaggtaaatttccttccactgaagcagccaggctgaattcgaattatctttgcccctgcttaaaaactaatgcaaattttcctagagaatatccactaattcctggagggggcatgggcattcctgatgcccatgagaggaccatttgctcttccctcagtatgctaaataacagaagcgacatttgttgctggaaagtatcagtgaagttaataaggtttttcttgcccagggtgagggaacagttcccaatgacaaatgctgtatgggaaggggctgtagaactgccagcccctttggtccatccgtaaagtgaactctgtggatcctggaggattccagcgtctttttttttttttctttttttttaagacagagccttgctgtcacccaggctggagtgcagtggcacgatctcagttcactgcaacctccgcctcccgggttcaagcgattctcatgtctcggcctcccgagcagcaagactacaggtgcgcaccaccatgcccgactaatttttgtattattagtagagacgggggtttcactctgttggccaggctggtctcaaactcctgacctcaggtgatccacccgcctcagcctcccaaagtgctgggattacaggcatgagccaccatgcccagccagcatctttcatttttctgtctgctttggccctttcctctctcactgtcttccttttccatttccaaagtcagtccatctcactattagcacaaaaactgctagagcgcttgtcattggtcatctctccctgcacctggctggtctgttcttggccactgaagcgtttcccccagctgttgctttaatcattttattgttattatgccttacttaagaaatggatatgagatgcatttacctgtctcttcctgccactctgcagagccagtaagatgtggtggaaagggcccaggctttggaggagggctggctggggttggatcttggctgccccctactagctgtgtgaccttgggtaagtagctggacctctctgagcctggttcggaatcatagcacctctctttcagggctgctgtaaggaatagcagtggtgtgtataaagcagagcgcacagccagcaactggcccctagccacactgctgagcacctactgtgataagctgccattgtggtgtgtgaagcaaaggggaaacatgcctgctgtagtgagcttcctgtagggcaggttgtagaaccagaggtgggttccaaggttacaaagggactcttagtgtattagtctgttctcacattactataaagacctacctgagactggatcatttataaagaaaagaggtttaattggctcacattggctgggtgcggtggctcacgcctgtaatcccagcattttgggaggccaaggccggcggatcacttgaggtcaggaatttgagaccagcctggccaacatggtgaaaccctgtctcttctaaaataaaatacaaaaattagctggccatggtggtgtgcgcctggaatcccagctactcaggaggctgaggtggaagaattgcttgagcccgggaggtggaggttgcagtgagccaagatcgccccactgcactctagcctgggcagcagactgagactctgtctcaataaaaaaaaaaaaaaagaaaagaaaaagaattgcaagaaataaattattgtttatgagctatatggtctgtggtaccttgttgtgggactgggagtcttggcgtctccctgaccctgcctgttgctgcagcaccgctcagccctgcctgctccctacctgcctcccctcggcctctcctgcctccaccgggcccctggtgcctcctctagagacagtcctcctgggaccgattgtgttctcacttacacgaggcatccaggactacagataaccagaggaaggggcgccccccccgcctgccctcctccctggcatcctcacgctgcagaggtcagagcctcatcccagccccttacctgcccctactctgtggagaaccgtggtcagttcgccaggccggatccacgaacggccttgtggaagatggtgagctcacacccagagctggctccgatgaccctgtctcctttacatgtttctaccttcccctccctaccttcccccactgctgggcgcagagtggaggcagatgaggtttaaagctcagaagggcttaaacgggttggggcgcagtggctcatgcctgtaatcccggcactttgggaggccaaggcagaggatcacttgagcccaggagttcgagaccaacctgagcaacatagtgagaccgcgtctctacaaaaaataaaataaataaaattagctttgcagggtggcatgcacctgcagtccctgctactcagaaggctgaggtgggaggatcgcttgtgcccaggagtttgaggctgcagtgagctatgctggcaccacagcactccagcctgagtaacagaatgagatcctgtctcaaaacaaacaaacaaacaaacaaaagaaggcttaaagggggctccaggtgggcttggcagcacaaagctatgaagttctatcttagacacaagttctgttactgggcctttgcaggctggcctgggtacctggctgccatagacagggaaccttccagatgagctgcaggcgtggagcacaggagccagggtgctcttcctgggctctgtccacaggcagaacgtacacagtctttgtacacgtccggcggctctggtgcctatttttgtttgtgtttttcttttgtttggggggatggatttggtttcccccgagccctctgtcctcctgtcacctggctggtgctcggcaatgttgaccagctgcctggctggagttggcagtggctaaggctgtgacagctaacatgttcctgagtcctctcatttcttcaccataatgccctgttgagtttgcagatactgtctctgtttttatctcccggggaaactgaggctcagagtggctaggccaccttcccatggtccctcagctcatgagggccacacagggcattgcggtggccttctcctcagccttgaccctccggccccagcattgctgcctcaaggggtctcctctgctgagccgtgcaccttctgcctggcagctccaactctgtggctgtgttcagtggctcagcactgccccttgaccctccctggccttctgcggatgccagactggagcactctgacaaggtctggggtggttgtatgggtcctgtgacctctatacacctcccagtgcctgggaatcctgcagatacaccctccttagccgtccctaaccatagaggacatttctgaggtccccgagagagtggggcacccctgcaggatccaactgctgggcccaggaaggatagcagcagcatgaggggttccattagccacaaactcacggcatggaaccttcacccacctcgcccctcatctgctgtttagcacctggcacgccgtgtatacttactgattattacattttaatggcaaattatagtggcaaacgtatgcatctttgcacaattgttgtacagcatgatgaacaagtcattaatagtaaagaataaatgtgaaagtgagaaaaatctgactgccaaagtttttactccttccttccctccccagacttttaaatgaaagtttagggataatcccttagttgtcctgctagtaggacttgcaattaaaagaattgggccaagaacacttctacgcttctccttttaggtttgggtgtaaattcggggtatttctcactgatgaaagcctggtgcagggcagaccgtgggaagctttcatttccggaatggaccatcaacatcccttggagaagaattctcttctccagacccagacctggtgtcctggcacccattgggcaagtgggtcctagaagacaaacctggtcagagcctggaggctgcttagcattccccacgcacattagcagctcggagagctcaggaagccgcagcccctccttgcctcaccagcctggatcaggacagcatcccctggaagacacacagggcctggcctctgattacccagcctggagggaaagctcaatcgagcatcatgtcacccggtgcccccatgcagggtggcactggtgagacccccaagccaatgataccacctcacaggagtgcaggcccattgtggccagatcatcttgacttttcaagataaatcagaaatcgtatttccatgagatatccctatttgcaagtgatggtgactaaattagaagtttttgaatattgtaacatgttcgtaggctgtttgtctggtttaaactctatctggaggaattcaagctagacttcaggaataacttcttgaggcaaggattttgagaccttagggaaagaaggacgtcttgggggtattctgactgttgtcctcctggaagggaagaacagagaactagaagactgcccttagcgaagttcaaagcacctaagcccgggaccctcagcaagtgttcttgagtcacagattctccctgaggcgcctctttctggctccatagaatggctgattctgtaactcggtgagtttgctttttttttttcctccatcacccaggctggagtgcagtgaagctggagtgccgtggagcgatcactgcaacctctgtctcccaggttcaagcaattctccttcctcagcctcccaagtagctgggattacaagcatgcagcaccacacctggctaatttttgtgtttttaatagagacggcccgaagtgctaggattacaggcatgagccaccgcggccagccataactctgtgactcttgttacaaaggccttatattttgctctttgagggtggttttggtttgatgcctgttggttgccatcttttaactagggatgttttatcaaaatgcccagccaaagtgtccaaacaaattataccttaaagtttgaaaatgtctggcacttctaattcaatgcctgttgtgccaggcactgggctgctgaggaactgagtcccgtccctgcaggctagctagagaacacacacacacacacacacacacacacacacagagtggtcttacaagtcagttttatattctacctatatgcaataaaggtattattatgttgaggtgccttgatataaaaatttttcttaaaggagaggatgcctaaaacaggcattacctgaaacctcctctctccagcattggttgtcttctgtcatgactcagggttttcactgagaatgggatggaaatgtggtctaaagatagggccaatgttgggactggatcccctctgggaagtcagaccaggctagggcaggtccttgaagccatcaggaaaagcctctggagccagaaacaaaacaaaaaaaaaatggtgttaactaaactcagtctcaaatcctgaataggactcaagtcaagcaaaataattaaaggagttagcaaagggcaagtcagagagaccgagcaacaccaatgtcttccgggagccctgtggcgagtgacagagcctggactctggagtagaactcatcttgtgtcttcttctgccactcgttagctgggtgaccttgagccaagccccttaacctcttggaccctatgttcttatctctaagtaggggctggtaatatcttcccctttgaggaatgccctctaaggggtgttgtgaagattcggtaaggtggcaggggtaggactcctggccagaaacaggcacataataaatgctaagtctctccttctctccacctgctggatgctgtagatactaaggatttcgatgtgaatgagacaaaacccctgccttccaggagcctttgagaatcagagaactagacccatttccagaacaaggggatgcagggtctggataaagttttggggatcaatagagcagagggctcccagaggatcccatagggttgactcctaactcaagggcatgagacaacccccaggaagggcaccctggaaggggtccggctgtccctgatttacttgtgggcactgggggaatgcccggagccatccagccctcagggctctgtgtgattctgggttcctcccataaaagataatcagattctttcacgttaatgtctttctccacctcattgcacatcatgcagctattcattgactcagcaagtatcagctttgcatgcgaccttggcctacccactttagcttttagtaatagctcccttcttgaataatacaaccagtggggaaacagaacctaactcttacctctgggaggcttatttgctttgagaacatatgtcctgcagttttgttcatatggcagtgaagtttcgtgcacacactctagagccaggcagcctgggttcaaagcgcagctctgccaggtcctaactgcatgaatttgggcaagtcgctcaacctctccatgcctgagtttcctcatctgtaagattggagcaatggtaatacctgctttttagggttgagaagagaattaaatgaattaagatgggtaaagtgcttagagtggagctttgcaagtagtaagtgctatgtaagtgttcgatttaaaatgaaagacccttaaatacattctttgttcatttcacaagcccttcatttcacaaccttacatttcacaaccaagctctgtctcccctggaatccagccataactctgctcacaagtgtgagacaggccccagcagagctgcacgaagaggagagaaggcagccccccagactcccaaccccctgtccaagatggcaaaaccagaacacagcctctgtaccaccccagcaggtattcagaatctgcaatctccaaagcccacttcaattgtaaatgtagagccacgtgcgctttaagtcacctgtcactctggaggctcttttgctcagttcctcaccattagcagggatgacagggagtgcaggagtgcggtcgactcccagatattggagagcgctgggctagctgcccattctcccggcctccactcctctttgctgtccagccatcacttgctctttgaaggcaaacaaaacagaaaacagtgccaaaagtatgggaagaaagccagcttctcccctggggtgcctgtgatgccatgcccaccctccctgaccacgcagcccctgtggaccctcagggccccaagcccccatttccatcacatgcgtacacccatgtgtgtccatagccgcccatctcagtcaataaggctgctcctgcccacttggaatagtggtgacaaccaggagtggcttatgggaactatcccaatggcctgacagcatgtccgctgcaaaccgctgaggtaggacactgccctcatgtctagctgatcagcaagaggcgcagttgctttcttaggtaacattgctgctgtgtcctggccattgctggggggtggcacttaatctacaccagaattttccctcctgtatcttccaagctgcttggatcttggtgctgaattaggttggactttgtcttgtggggaagggaggactatagaccctcaacgtaagcaatggtcagactattctaagaaaactcgccgaattaaagcatgaggtaaatttagttctgacttctgtccaccccactgccactgtccccttttatcccatgatcccttgcttttcttttcctcctctctccctatctcttgtgtttgacgcatgataggaattcagaaatatatgtttgtggatttgtttattcacgtagcaaaccatttcttgagtgcctaccatgggccaggtagaatgggcggccccgggctgcagtggtttcttcagcccctctccagggtttacactgtgcaagacggtttgtgatgggtcctcccatcgaggaccacactcttctttctctgtgccccttggtcctcagtctctgaccccacttcaaaggcagcattcactcagggaagctcccatacaatgctagtcagagtaaaagtttggacaaattgccaggaagcagcttgtcagtatgcataaacagcctttaaaatattactactctttgacccagaatttcacttctaggaatctgtcctaaggaagtagtcacatgcaaaagatttatgtaccaagatgttcatcaaagtgttgttttataacaggaagtctcagaagctggataaatatccaacctctggaaatggttagatagaatagtatgtagccattagaaaattatgtctatggggtttaaaatgtcatgggaaaacacttctgacataaaagagcatgagaactgtatatttagcataatcttaactatgttttagaatgcacaggaaaaaaatgtacaaacatattcatagtgatgtctctggtggtaggattatgatcagtaagtacttctgtctcttcatattttcctgtatttgataatacatgcatatgttgtttttaaaataagaaaaattttaagtttaaaattggagctgaaaagtgtttttaggtcaggcgaggtggctcacacctgtaatagcaccactttgggaggctgaggcagtcagatcacttgagcccaggagttcgagaccagcctggccaacatggtgaaaccccatctctactaaaaataaaaaaattagccatgtgtggtggcacacatctgtaatcccagctacttgggaggctgaggcatgagaattgcttgaacccaggaggtggaggttgcagtgagccaagatcgtgccactgcactctagtctgggcaacagagtaagactctatgtcaaagaaaaaaaaaaaagaaaagcctttttaaacagtagcagacataactatataatccttactaagctgtcggtcaaatttttatttatatatttattttattcatttattatttttagacagggtctcactctgttgcccaggctggagtacagtggcgtgatcatggctctcttcaaacttgacctcccgggctcaagtgatcctcccatcttagcctcccaagtagatgggaccacaggtgcataccaccacacctggctaattttttttattttttatttttagagatggtgtttactatgttgcccaggctagtctcaaactcctgggctcaagctatcctcccacctcggcctcccgaagtgctggggttaccagcatgagccactgtacccagccctcaaatttttaaaaatctataagagacattattggacaattagagaaattcacatatggacttataatagtatcagagtgtgtggtgtgatggttctggagggaatggactttttctttggagacaggcttttctatgcccacccttttatcttgctaacttatcatcatccaggttccagcagaaacattacttcccccaggaaatttcttaagggtgcagtatcatgatgtctgcagcaaattctcaaatagctcaggaaaaaagtacgtgtgtggtatgagtgtgtgtatgtatgtgtgtatatatatacacatatatacacatatatatacatatatgtgtatatatatacatatatgtgtatatatatacacacacatacacatatatatacacacacacatacatacatgtatttttatataattatatatgcagagagtgcaaatgttgccaagttaaagattggtgagtctaggtgaagggaatatggtatttattgtattatttgtgcaacttttcttaagtttgaaaattttcaaaacaaaaaattggaggaagaaggcatgccagtctaccccaagccctccattggaatgctgaaaatctaaacaatgtgatttggcaatttcatttcttttctgttgtgggccagtagtccttagatgttggggaagggggtagtcgctgaggtgtggttgacttaggatggaagaagcagaagtcaagactcccagggtcaaagtggtttgctctgctgacccaagtgtgggaggcccagagtcagcgtttcaggtgtgctaattcagcatggttctattcacggccaaagtccaccctgggcacctctctggcagcaatcttgggtgactctactaaggccaggcctccatgaccctatgtctggatcccatatctccacctctcccactgtctcaggaacggtgcttagctttttcttttccctctcctgtcttctttgccagcatgtagaaagtttaaataattcccctctttacaacaaaacaaaacatacccccttcagtcaaccaccctagctctcttctccttttcccagccagatttttttaaaagcatcctaggccaggcgcggtgactcacgcctgtaattccagcactttgggaggccaaggtgggtggatcacaaggtcaggagatcgagaccatcctggctaacatggtgaaaccccatctctactaaaaatacaaaaaagtagccgggagtggtggcaggtgcctgtagtcccagctactcgggaggctgaggcaggagaatggcgtgaacctggtaggcggaggttgcagtgagccgagatggcgccactgcactccagcctgggtgacagagtgagactccgtctcaggaaaaaaaaaaaaaaaaaaaaaaaaagcatcctcagcactttggcaactccatctcctcccaacatgtccctgttactggaatccagccaggactcagccccgatctttctactctaaccagttgtctcagttaacaaggacaggtttatgctgcagtgacaaacaagatcccaaattcttgtggcttcacacatctggcaccacctcatcttccagccttaggagtcatcttttagttccttgaaaactctttacagttttctgttggggccttgtcatatactattcccctggaatgttctttcctatcccctccctttcaccttgctaacttgtgcccatccttcaggtctcagcagaaacatcacttccttggggaagttttctccaacacccacactacacaggtgtcccatctacactcctatgactttgtggtacttgtctcacttcattttccactgccttccccacaaggcacctgcacaagggcaaggaccgtaccactgtacctatgtcactcattgctgtggtcacctgcactctggctgcctaccttaactacacattagaatcacctgaggagcttttaaagccacaatgcaagactccaccctaggccaattggatccaaatccctggggtagggccagacatcagtggagttatatatacatatatatattttgtttgtttgtttgtttgttttttgagacagagttttgctctgtcacccaggctggagtgcagtggcgcgatcttggctcactgcaagctccgcctctcgggttcacaccattctcctgcctcagcctcctgagtggctggaactacaagtgctcgccaccacgcccagctaatttttttgtgtttttagtagagatggggtttcaccgtgttagccaggatggtctcgatctcctgacctcatgatctgcctgcctcatcagcctcccagagtgctgggattacaggcatgagccactgcacccggccatcagtggatatatttttaaagcactgcagagaattctgttgcatcagcttgagaaccactgatctgccttgtgcttcacatttaaaacttttttttaatgaataaataaaccccaaaaaattaatctccctaagcctccctagaagataggatggtaaggatattttcctaggtaaaaatatgttaatttcatatttcatgaaatttcatgtttcatttcaatcaagctctgtcatacaccttacatggggcaagcccagtgcctgggcagggtgtaattatactcattacacaggcaaggaaaagtcacattaggtgatggagcacaaataggcagttaatggtttcagggctagttaggatatgtttgtctttcaattgcaagtaatagaagcccaaagaaattggttatttatataatataattgattggttcccaaatttgaaaaattcaggaatagacccagcttaggtacagctggatccagtcactcaaacaatgtcacaaagaaccctttgacaggaatgtatcctgtgttgactctactttgctctgagtagtctttccccaggtgatgataaaaatggtcatcatcgccaggcttgtgtcctgtttagtaggaatatacaagaagagctcagtaaatgctggccccaccactaagcaaaaacaaaacttttgttgttgttattgttgttttaaataacagcttagacctttcttctttccttgttattctctttcatctgtaatccagttttctacttctgaagtatagaatgttctgatgatttattcttcattacccacaacttgcacatgtttatttaaaaatgccaggattgcctggccgttgtgtgctgttaacctttgtttgctgttagtggatccctgaagttcaggctcccaggggagcagataatgggtatccagttcctgcaatatccaccctctggcaagccaagttccttcctgggtaaggttttgcctacctgcattcctagggaagtttctgggcctgaccaccaagccagctctgagaaggggtgcataagccccaccatgctttggctctgtccctatagaatattttatgttgttactgaaaactaaaggaagatgggtgcggtggctcatgcctgtaatcccagcactttgggaggccaagacagattgatcactcgatgccaggagttcaagaccagcctggccaacatggtgaaaccttgtctctacaaaaacaaaacaaaacaaaaattagccgggtatggtggcatgcacctgtggtaccagctactcaagaggctgaggcacaagaatctcttgaacctgggaggtagaggttgcagtgagccgagatcgcactactgcattccagcctgggtgacagagcaagattctgtctccaaaaaaaaaaaaaaaaagaaaaggaaagctaaaggagagagactaaaatgatatcaggttcctggagaacaaacagacatgattttgcttcatggcaggacagccggaagaagtgggattatatcctcacattacaaataagaaaactgagactcagaatggttaagtcacttgtcccaggccacacagccagtaaattacagaaacagaatttgaacccaaatcttccagctccaaagcttgtgttcttttcactacctcctgcttaattttttaatttctaagattagacccttcatctatccatgacacctgcctgtcatcccctgaaaaaaggtgaacgccgttcagaaatttttctagcctgagctcactcccagttcacttatttttgctttgtcatggctgcccagtccccacttgtagaccaggaataggtcatggctgcggggactacacgctgtcgctgctgcaagggccggcctctgtttccggggctgagtgggggccagacctgccaggagcaccatcttctgtgggtcctgcctggatgtcacatcccggccccaagaagtcactgcaaaccttcgtattattgagcttcacatcctagaatttgctgtcactgtggctgctgcatgaagttgtcctgagagaaacgggcattgtcattaacagggaaattgatggtctgggggaaaagtcatcctcattctcttgcagatctatgggtgattgagactggctgatgttgaaggggtttctcagccatcgtgtgccatgttatggaacagtggtgtagccagccatttgacacccagcgctgacctttgtttaacaacctcacctatatatgacaaaatgattgtcagaaataatcgtgtaatgaaatgactgtaataatggccagaaaagaaacgcagatagtaaaatgtttctcttgttgaactctgtacatataattgcaccaggatttttttcaaataaaaagtaaatattatactacaaaaaagggaaaaagcacaagcatttattaaatagctttctatatctttctgagttttgatcctttgattgcagactgatgtaatattttatgtaaatcattgcttggttactaagtgaactttaagaaaagtgagacgtctgcagaagttgcccataatttagcagctactgtattgtaccattgatgtacggctttattttcttgattaattatttaaacaatataattcacaattttaaaataataaatttccacttaaaatggtatttaaactcagcaaaatatatcatctatgagtaaaatttgtatttaccaagcaaaaatattacagtttgtggttcacatgctgtctcactgttttaaattttaaatacaaaaactccaagtaggctgggtgtggtggctcacacctgtaatcccagtactttgggaggctgaggcaggcatatcgcttgagttcaggagttcaagatttgcctgggcaacatagtgagatcctgtctctactgaaaacaattagctgggtgtggtggcacatgcctgcggtcccagctactcaggaggctgagataggaggatcacttgaaccctgggggacagaggttgcagtgaggcaagattgcaccactgcactccagcctgggtgacagattgagaccctgtctcaaaaaaagaaaaaaaaaaaagaaacacaaaaactccaggtggtcgcacagaatgacaggactgaagtaacttagctccaatttctgtcttcataatcactgtcctaccattgtctgtgcttagaatctacttgcttaatgcaggaacatgtgttctcacagagatggaaaatgcaaatggcgccagaagcaagctggaaattctgaaccattaagaatttactctctgccaggcacggtggctcacgcctgtaatcccaggactttgggaggctgaggcaggcagatcatctgaggtcaggagttcaagaccagcctggccaacatggtgaaacttcatctctacaaaaatacaaaaattagccaggcatgatggtgggtgcctgtaatcccagctactcgggaggctgaggcaggagaatcgcttgcacctgagaggtggaggttgcagtgagccgagatctatctgcaccattgcacttcagcctgggagacagagtaagactccatctcaaaaaaaaaaaaaaaaaaaaagaacttactctcaaaataaatacgtgtggctgactccacatatggtagggccaactgtataactagaagttctccaaataacttctgtggagaaaaaaaagtttattaaaggttaacttttttaaagtgctaactagaaccttactaacactgagatcgcaccaattgtttataacttagacagggccgggtgcagtggctcatgcctataatcccaacactttgggaggccgaggcaggtggatcacttgatgtcaggagttcgagaccagcctaaccaacatgatgaaaccccatctctactaaaaatacaaaaattagccaggcacggtggtacacgcctgtaatcccagctactggggagggtgaggcaggagaatctcttgaacccaggaggcggagattgcagtgggccaagatcgcaccattgcactctagccccagcaacaagagtgaaactctgtttcaaacaaacaaacaaaaaaaaaaacctcttggaccaggaaaatattttttaagggaggagtattttatcactggcattgtttaggattgcaggcacatgatgctaatgaaaagcagactaactattagttggttttattactgtttttgaactctctctctcccttttttttttttttgagacagagtctctctctctgtcacccaggctggaatgcagtgactgcagtctcagctcactacatcctctgcctcctcagttcaagtgattctcgtgcctcagcctcccgagtagctgggattacagggcaccacaccaggctaagtttttgtatttttagtagaggcagggtttcaccatgttgcccaggctggtctcaaactcctggcctcaagcgatctgcccatcttgacctcccaaagtgttgggattacaggcgtgagccaccgtgcctagccctgtttttgaactctctagagacagtccagccccttattacttgtcctgaggcagctgctcccttcacctggccccccgcattgtgttccggacccttgtcctggtggtgctaaagaatatctctgtcgatcctttggggactggggaaactgaggcccagtgccacgcgatgccatttgttcagggaagattaggtcatctgctaggtccccagtcacttgaccttcttcccagacaggaagaagctgctctgggtctctcagtgctccacgtgtctttgcacattgaaatgttttctgattttttttttttttttttgctgttacatttacttttaaaaaataacaagcaataaaatgttacatttgagaaggttgaaatgagaattgatttgagttaaattctagcagatttttcttagaagaatgatatcatcatctccagctacctgcaattgatctactctgaattaagaaagagacttccatttgttgtttatattttgcactcttgatgtgtttctttaaattatggtcatgggccaggtgtaggagctcacacctgtaatcccagcaccttgggactctgaggagggaggatcactggaggccaggagttcaagacctcgtctgtacagtaaattttaaaaattagccaggcatggtagcattcacctgtagtcttagctacttgggaggctgagatgggaggattgcttgagccagaactttgaggctacagtgagttattttcacgccactgccctctagcctggctgacagagcaagacctgcctcaaaaaaataagtaaaaaataaattaaatttcaatcattagcagtcattaggatatttaaatacagtatgttgaatcaaagttacgcatgtgtgtatttttttttccagagagttgtttatcatgtgggttttaatttaactttaaaaaaatgttggctggacagttgcccaaatggtatcatcagccatttggttgagaacgtatgtcctgcgggctcctctgtcactggagttttgctagctgacagccactggctagttagagactgcagtcagcacagatgcaggcgtggacttgcgcacgtaaccatgtcaatgcaaagccatcacttcttaaaaattctgaaccctgctgtctgagatggtggtgcagcggatagaactctgctctaagaggcagtagctaattccatgtcttctttgcccttgactagctgagtgactttgcacatggggcttgcctctctgttgccttgtctgcaaagtggaatcatcttttccttgctagacagaaggtggaccctggacctatggcctttttgagtttcccccccgcttcttagaaggacctctgatcctactgagtttaatacccacgggttaataattgggaaaagcaaaggaagcgcttctgtttaggtaattatatgcatgtttttgtctttttctggctggaaagatatccaagccactgggaaggtccgtggctacccagggtagccctctctggggagggctgctatatccaagagcccctcatgagaatttgaaaatcgaccatggtagggcctgctgacttttgacagctaatggtgtgctgagaattgtccctccaaagatgcctttccattccctcgggagagtctgggcagcccctactgggggctgggatgctggctcttccctcagcctccaccccaactgctctcttccctcctcccctccccagccccctaatttctctcacaaggctttgttctgcagcaacctttcctaatgcagtcctggcctcttcgcagcttcattacataaccttccgtggactcctggtccaaggatcaccccagaaagccagtcagaggtaggcacgcagctggggtccatttacttaccttccccaccccctcggaactcagaggtggtgcaggaatttggactccaagaattaacagctccaccaccatcaccagagccaaaactcaggatgcatgtgcttcatctgctgcttatttccagctgagagccagtggtgccatggttccttagggagccggtcccctgatgccggctcctggccccaaatctctctgatccgggctcttccagaatgtcttgtctccaccatcgcctttgaccaatggtgtccctttgcctggtaatgtcccctttgcctgatgatggccctgtcactcctctctttagcacagaggaggctgtttcatcccttcaagcctgccctcccttcaagtcttagctcaagttcaccttctccgcagagccttctccaatcttcttgactacgtctcctctcagctccagcaacctctgtctctggcactgattccttacttagctaagagaatcacagacacttggggctcaggacaatctgctttctctcttcttacccatggccttggactgtgtgtacctctttgtctccactcccaaacccaacccccagagggcagagagcatgttgtctgtccctttgctcagcatgaagccatgcgtgtggtagatcggcagagttccataacttgtgttgaccgaggggtcactttgctctgaaattacccctgtgtccttcagtatttgcacagatagcttcctggccagaccgaatatatccaagggcatggcccacctctgctcctgtttccaggtccctggtgggggttagttcatgccttcctcataatctgcccactggcctggtcctcaaggtcttcccaactgctcagccagagttgagaaaatgggtcgctccatcctgtttgtgtcgttctctccttcctggcccactctcctgcccacaggtatccaggggctgcctgtagcattagaggacatacatgcacatgcgtgggcatgggacactcacgtagcctccaagcacagcatcaataatgcattctgtgctttatagcatggaaagctgctctaaactttattacacagtggacatgtctgaagcagctcccaaatccacccctgagtgtgttggaattggcaagcctatcacttgggagtctagtttttttgttcgttaataatagatgcttcctgtggccccagcttggcaattttgatttaaagtgatcttaactgaagagactaatggacgggtctgaatttgtgccttttaagcacaaagtattgctcttaattaactggattctatcctttgagcaggcagaggccttcccccaagggcgtcattaacgatccacatctggacatcttccaaagccttcttctgtttcaggccaaccgcaggtgtgttcctgaacacccaggaggctatgagagccacatatgcctcccaaatacacacagtgtgcatgcccagggacatagagcagtgtgcaaagtcccattccatctctctccacctgggagaggatggctcttctgtctgattcatggctcaaagtggtaaaggagctccccactccccgtcccacgcctactcagagtctgcaaatatgtatgcgatatgagagctcgtcagttagctgtcttcagtgtggcgcacatttgaggagtctgactcccctccagcacaggccaatgtgcactgctctcctatctttgtacccccactgttgcactgtgcagaggttggagccatagaagtaccagagctgtgaaaggagaggccccctctcacctctgccctggtctccatccccactttctctaggaagctagtaggtgctgacaggggagagaagggaggggaggggtccagaaacagtggctcatgcctgcaatcctagcactttgggaggctgaggcaggaggatcatttgaggtcaggagtttgagaccagcctgggcaatgtagcaagaccctatctctacaaaaagaaaaaatgtaattagctgggtgtggtggtgggcacctgtagtcctagctacttgggaggatgaggtgggaggattgcttgagcccaagagtttgaggttacagtaagctgtgattgcaccactgcactccagcctgggcaacagagctgagaccctatctcaaaaaaagaaaaaaaaaaagaaaggagagagagagaaagaaaagaaaagaaaaaaaaaaaagaagggaagggaaagcccagaagagtgtggggagaggaggcggccgtcattctggggccctcagtgtgcacaaccagataacacatgctctgtgggcttttgtaccattttgcttgagcataaagaaaggaaggctgcccctaaatagaaagcactctggaggcaaacaaatctgactccaatcctggccctgccactttcccagctgaggacttagacaagcaccctagcctcttggacattctcagagccatctgctgcaagtgggtgctgccatacccaccttactgggcaggcttgggggaccaagggtggtaaatggctcagtctttcatgatgcggccacacagcaggtgcgccatccaggtccatttctttccttcctttcccccaaatcaagttgtcattaaagtactagtccacattaatgaaatcaactgtattaattttctatttgctgctataataaatcatcagaaatttagtggcttaaaccaacacaaatgtattaccttacagttctggaggccagaagccctccataggtgtcactgggctgaaatcaaggttttggcaaggttgcggtcctttctggagggtccaggggagaatccattttcttcctttttccagcttctaaaggtttcatgcattccttggctcatgatcttctatagctatagtcagaaaaattttccatcaatcatcttcaaagccagcaatggcaggatgagtcctcacatcaccttgctctgacaccagttctctgcctccctcttccacatgtcaggaccctcatgattactttgggctcactctgataatctgggatgatctctctattttagagtcagctgactgggaaccttaattccatctacaaccccaattcctctttgccatgtacagtgacatattcacaggttctggggattaggacgagcctgtctctgaaaggctactttacatgaaaattcatttttttaattaagatttttttttcctcttgagacaaggtctcactctatggttcaggctggagtgcagtggtatgatcacagctcactgcagcctcgacgtctctgggctcaggtgatcctcccacctcagcttccctagtagctggaactacaggggtgagcccccatgcccagctaatttttttttttttttttttttgagacagagtctcactcagtcacccaggctggtgtgcagtggtgcaatctcagctcacagcaacctccgcctcctgggttcaagtgattcttgtgcctcagcctcccaaggagctgggactacaggtgtgcaccaccacgcccgactaatttttgtatttttagtaaagatggggtttcaccatgttggccaggctggtctcaaactcctgatctcaagtgatccaccaacctcagcctctcaaagtgctgggattacaggtgtaagccaacatgcccggccccagctaatttttaaatattttttttgtagagatggggttttaccattttgtctaggctggtcttgaactcctgggctcaagcaaacctcccaccttggtctcccaaagtgctgggattacagcatgagccactgcactcggccttaagagaagatttaataattaatactttacaacaagatctggaagaggtgggatgagtaactaaatgaggatacaagtaacccgggtcatatttgctaatacccttggtcacattgaacttgatatcttatcagattttcctaatcagctcctttagcagcagtgttgcagcatcttatctcattttgttttttgtttttttgcctagcacatgcctgtaaatcactggattgaggtgtttagatgtttgttgtcctttggatgcttcttataaatccatatttcatggctccctggaaagtgctatgcaaatgataagctgcaaggatggaaaggaaattgcagtgctcctgaattgtaaatgggcttttacgaggaggtttctaattactcgctctttctcttgaactgaggagttgaagtgtaggtggcagatccataacagataatcatgtgtgtgatgtgacttcagcctgagcgtcgaggaccaagtcacagagcaggaacagccactctccagtgtccttggggctacgtctgaggagaacctgggatttcatatatgacctgcactggctggggggctctcttgacgtaacgtgttccctctgagcatgttacagattctgacattcttatgttccttctgtggagagacatgtacttagtgacctaactcactttagcatatttttgctcatcgtttgtgtagcttaaaggaatcagataattaccccctccccactactttcggaagcacaaatgcaatgccctagaattgtactggggactcaaaaagaaaagagagtagtaaaatctattaaaggggacaaagacagcctatatactacaagctttctatttttatggcagagaatgccattttctaagtaaacagagaactgcatttgacctgcaatatcaaatgcatggatttgatgctttggaaagcaactgttttctgcgttaatctgggtgtcttccgtgaaatgtcctcctgcctttggcttaaacactagctttgtctacagccattccatcctgaacctgcccaatcttgtctgaatcctggtttcaccactgacaagctgtgtgtccttgggcaagttacttcacctgtctgtgcttcagagtcctcatctgtgagttggggaatctggacagaatctaccccatagggcgtagtgaggatgtgttgaattatcccaagtggctacacagagtaagcactcaaatgatgtcatcgttgtcatgattgctgttaccagagcctagagttcattctgatactcgagtctgtggcccatccagcccaggtaaggaatagttggaggagttgggcatgttcagcttgaagaggagacgacaggggatatgggatagttgaatctgtgaagggccccctgggatgaagaactggcatgttctgtgtggctccagggcactgagcaggacccatttgccaaagtctcagggacacagtttctagctatagacagaaaaattttctgtcactcagaggatgaaaatagaatgagcccccttaagaggtaatgagctccctgtcattggaaggattccagaagagctaggtaaccactttaggtgctatcaaggggcttttttctttaaagtcctttccaaaagcttctgagattgcataaacaataggaagccatcttggtgctttaacacaaactctccccagtgatgagggttgagccaaagccagattggcaagcagagaggagacttgtgtacaaggagttcctcgagtcaattgctttttccttgttctagccagccagagggctcctgttggaaaacaggagaccggagaggctgaggcctgaccaaaccagcttctgcaggccagctgggaggccacaactcctacctacgggaaaactgaagggcatctctatttttagattagcaaaagaaaataaatttaagtttgagtctcctttgcaacttttaaaagacatctttattgagatgatcattcacattctataaaattcccccactttgagttacaattcagtggttttagtcttccttgatgattttgatggtcttttcttaaggctcttggaagacccagaagcctctcagacacaggtgggtgtggagggcgtagcacagaggcagacttctcatttcctgggtctcccctttaatgactctcagagacccctccttccccctgcccctggcttctaccccaggggtgtagagttttgccattttccaagcagaacttcatttcctcttctgtgtctacactctttgtgcttctttcttgccagctttttctcctttgcccgcccttccttccttccttccctccctccccccttccctccttccctctttccctccttccccccttccacccttccccccttccccccttccctccttccttccttccttcctgcctgccttccttccttccttccttccttccttccttccttccttccttccttcctggtatgtgactaatttctgtttcaggacataaatgttgtccaggctgttctttggtctttctgttggataatggacatttggcattgagagaggctgctttttctgaaatcatgttcttggggcccagaacctaggtgtgtgcttctgactttgttttcttcctgatccaaattctgatatgtccatttaaattgatctagacccacagggcactgtgggacagatcctcagtggaacatgactctgtaacgagagcattttgttttgtcaaaatgagaacatattattgcctttcatctgattgtaaacataatacatgtttataaaacagtataatgagacaaaaatgtagacactaataagggaaaatctccctaattgtatttctcttcacagagaaagcccctgttgggcatatatactctagtttgtttatttgtttgactacacatatatgtattcttttcttatgtataaaaattctgaacatgcacatttctgcaactactgttttcacttgatgatgcatggacctctctagagtgtacgtttcttcttccttacaaagcagttggcttcgcccagggtgcaccaggacacggttttggctctgtccccagggtgtcacgggaccaggggatgatctcacagggtctgccatctgccctgcctggccggaggctgcatcgagagggccaaggggcaccacgtgtcgtgggtactgtcaaacaagagccttcagagccttccacagtctttcttttgcttcccagcattgcttccccgctggtggactctgaatctagaactagctccaggcgcctctccaaattcagacgggagctggggcactattataatgcaaatctaggcaaagccctcccaataccaggatccagaatggggtggggccctttgccctgaaaagctgtttagtttgaaaatacaaacaggagacagaaaagtttggctaaattaatggataaagttttaacgatggtaaccatagtagggttcatcgacagccagcgatggttctgaacacttgacatgtattaactcacctaatccccacattttacagacaatgcaaaggaggctctgggaggttgagtgacttgccccaaagtcgcacagctcctaagtgaaggattcggagtggactccaggcagcctggtctgactccctgcactgcgctgtgcttatctctggccccaatgccgccatgcagaagtgtctgggggcactttgtctctgtcagacagaattcggagatgtgtatgcttgccctggtatggcacttctctttttttgagacagaatctcactctgtcaccctggctggagtgcagtggcatgatctcagctcactgcaacctccgcctcccaggttcaagcaattcttgtgcctcagcctcccaagtagctgggattatagatgtgcaccatcgtgcctagctaaatttttgtacttttagtaaagatgttgttttgctgtgttggccaagctgatctcgaacttttggcctcaagtgatctgcctacctcagcctcccaaagtgctgggattacaggcatgagccaccatgcctggcagtgtggcacttcttacgtgtgttcagcggacactgtttatcttctgtccctccaagacggtgctgagctcaggtcgttcattactggcagacaactgctgatttccaacagaattgccatcctcttctcccctgcgactttcagagtgtgacctcagactcaaaaattagaagtgaaaacatcttaaaaactatcaccttttcttcctaatcctcctctcccctccctgtcttccttgttgtccccatctaatgaactatcatggcaaaaagagcccatttctggtcattttctgtggcctttcaaactcccacctaccccactgctcctgggtgcattacccgaaagctgagacttcagtgcagaaagtgccaggccctctgtccccccagatcgccttccttgtcttccctgtgcttgcctgtcacattgtgtgggttccagcgctggaaggaatgaggaacagattctctggttctccttttgaagtttaccttcgctccaccacttctgagaccttcccggaagttgccccttgtttctctcctctccagggctgccccagagctgcctctcacctcttcctgctgtcaccccaccaccatcagggcagaagttgggacaaagcctctcctactggctcctgcttttctcccttaggtccagcctcctcttctccatcttcaggagtctccttctccactcacacgtcatgacttcagcacctcgcatcagtccagaatatgactgcttgttcaagtgccacctttctcatgcatttttttctagtgacaatcacagccaccctgtggggcaggagtgtcatcatccccatgtttcaaatgaagaattgcagttcagagagggcaagtgactggcccagcctcaacagctagccagtggaccccaccagggcttctgactccagtccgggttccctttccacccaaatccatggagggagctgagccgagaacaggtgtccttcaggaagacgtgaagccaaagcctccacctccaaactcaggggcccagggagtccaggcacccatccactcacaaggctggatatggtgcattccaggagaggggttgggggcgagtggcctctctgtgtacccgtggggatagatgcgcaagtggcatcgccacatcgtgagtcctggcttcatgggtgagctccaggtccaacgagaagccaagcagggggcccttcaagctcagctttgggcccgggtcggggtacagggtagagcgggcctccccagcccctgccatgaggccaaggcagtgcatcgttcgcagcgtacattcagaaaccaaagcctaggagctggttatcattccggtttacagctgatggaagagcaggtgcttccgagaacccacagtgctctttggccagtgacccaagggtgcctctgagaggcctcgcagcacccggaggtgctgctgaggcaacgccctgactgtaagaaggaccattcatcctcagagagtggccgtgatgctgctgcgacagtcccaccatccctcccgactctcactcccaacagacttcccactgtaaagctgaactctccagcaaatcacctctcgccagactctctcctcactctctctgggtccactagaggttcctcagcctctctttgccttggttttcccagctgtaaaatggagcaaagagggcctatgtacccacaaaggtgtggttggagcgactcctcctacattagggcctcgagtggggcttcatgattggttggtggaggtctccaaacccacccagtgccaccgaaggctgagactgcagatgcaatgccacaggtgtccttcctcagcctgggcagctgaacatcatgtgtaaaacggggataataagataataacagccccttgcacctatgtggctgtgaggattaaacaagataaatgtgtaacagtgcctggctatagaaatatttactcttgttattaagggaagaatatgtgtggctaaaaagggatcgaagatgtaaaagccaatccctccccctctagcatatttaagggtaatgttgagttggtttgtggaccatttgctgcctgttagagctggaaggtagggaccccctctcaacagcgatgctacaaattatacccattggaggtcaaccaaaagacaaagcttattggctggacatggtggctcacacctgtaatcctagcactttgggaggccaaggcaggcggatcacttgagatcaggagttcgagaccagcctggccaacatggtgaaaccccatccctactaaaaatacaaaaattagctgggcgtggtggtgcacacctgtaatcccagctactcaggaggctgaggcaggagaatcactagaacccaggaggtgaaggttgcagtgagccgagatcgcaccactgtactcaaaccgaggcaacagagggagacgcaatctcaaaaaaaaagaaaaaaagacaaagcttgttaataccagcatattgttaagggaataaagtaggctgcagaacaactggtgtaatatggtgccatgtagggaaaattacatgtgtgcataggagaggggtctgcaaggttgtgccctaagatgttagagtggttcctttgcttttctcttttataattttgtatttgacttttaaataaggaccataaatcacttttataaaatacattctctccagcccctactactcctttaaagaataagagtggtttgcccaagaaagacagttttttttgctctggttttcttgattctgacatcagaggaaactgcttctcatccacttggggctctgggttcaggggattcatttcaggcagattaaagtggtgaccaggggcattcgtggacacagggagggacaggagcaccatcagtttgtctcacacaaccactgtcatcctcactgaaggctgttgcctgatcaaaaacagtattgggccaggcacggtggctcacacctgtaataccaccactttgggaggctgaggtgagtggatcacttgaggtcaggagttcgagatcaacctggccaacatggtgaaaccttgtctctactaaaagttcaaaaattagccaggcgtggtgggtgcctgtagtcccagctacttgggaggctgaggcaggagaattgcttgaacccgagaggtagaggttgcagtgagccgagatggcaccaccacactccagcctgggcgaccgagggggactctgtcttaaaaaaaaaaaaaaaaaatatatatatatatatatatatatgtcaaaaatggggtagtttttagatctatagtagttctaaaaacaaaggccatccaagcatgacagatttacaagcactattggctattccagtagttacaatggaggagagaagcttttagttaaaacaaacaaacaacacaacaaacccagaaaccttaggtcaaaaccaaaattgtcctctcagacacaatctgggaattttctcatgacagtgggcattagccaactgacatcagcagcaaccatccgtgtgcacacagtggcaccacctcctcccaaaaagcagccttcatctatgccctcatacaatcgttgattattctctttggattgaggcccggaattatttaagtttcttcttgccagcatgagtctttcctttctgtatgctccttatcttctctctttaatttggcagttctgcttgaaatctgggtctttcattagtagtagttcaatttggttccagaacattctgtggtgtgatgcaatgtgaccagagctcacacttcagagctcttcaagggccagtcttactgagcacctcccagtggctgcctgtgtgctgggcgccacttgtggtgggcaggagagaggaggggacacaaaaggagacacagctccttcttagaagctcaaagttggggaccagctgccacagaagagtatgtttagcatctgagacaccaagatccagcgtcacaagggtgtttattaagcctcctcatctctttctttttctttttttttttttttttcctcaggcagtcttactctgtcacccaggctggagtgcagtggcatgatctcggctcactgcatgcaaccaccacctcccgggtttaagcaattctcctgcctcagcctccccagtagctgggattacaggtgcccaccaccacacccagctaatttttgtgtttttagtagagacagggtttcaccatgttggtcaggctggtctcgaactcctgacctcagatgattcacccacctcggcctcccagtgtgctgggattacaggtgtgagccaccgcgcctggccttgctgttgattcatctatagtatgtttgacttgatgacctccagttaccttagacagaggttctcatctaagctccaactttccatttcctttgtcctcgtctttccccttaacccctccacatttctctcaaaatcaccccacttctaaaaaatactgtttatttttcttttaaatttcaaattatctatactcattgaaataaatcaaaatagcatggaataagcgaaaaaaatggatcccacccttccccactcccattccctagggctaaccatagttaaccatttaatgactaggtttttttgttgttgttattttttatttatttattttgagacagagtcttactctgtcacccaggctggagtgcagtggtgtgatctcggctcactgcaacctctgcctcccaggttcaagcattctcctgcctctgcctcctgagtagctgggattacaggtgcctgccaccacacctggctaatttttgtacttttggtagagacagggtttctcaatgttagccaggctggtctcgaactcctggcctcaagtgatctgcccaccttggccttccaaaatactgggattaaggtatgagccaccgcacccagccctcctgggctcttttcctttagttgcactcgctccccgctcctggagtagagggatttccgagagactgtgggctccagccttcacctaggcccaggactaggatgcctgccctaacatttatctttataccttaaagcaaaacagctggaccataagcattcaagaacaaactgtgaataaggagaaagttctcccaggaaacaagagctttagttctgttgggccagcccttatattccttagctgttaccagtcactgcttgatttaatctcggctatcacttggcctgacaggtctgctgctggtgccaggatgtctgggttttgaagcctggctccattacatacttcctgtgtgaccttgggcaacttactcaacctgtctgttcctcagtttccccagctgtattatgtcagcataatagtttgttgtgtgaattaaatgaggtaataactggaaatgcttcaaacatggttcctatcatgagaaatcctgctttccgcctaaatgtgctggaaaattcctggtggtgcagaacaggagaccagagcaaaggaaagacagggtgcagaagccaaaaattaccttggagaacaaagcgcatgttaaggttatttttggattctaggtttatctctgcttggtcttcagttacctgcaagagatccatttaggggatttttgtttgtttttaacgatagctttattgagatataattcatatgccataaaagtcactcttttaaaatgtttccggtatattcacaaggctgtgcagccttccctgtccttgattccagtctgagtttttaactgaagggataaggaggaccacgctttccccagaccagaaccgcgggccagggggcgattctgctgagtcaccgcgggcgcctggtgcgcggcggcggagcccgggaccttccttggctgccccctagcgagggccgcagcgcagcctgagacacccgccggggccgctccacggccgtcggatttagactggaagctcggtccaggtccccagcttgatgcgcccgcggtgtaggagaccagcccgactcgggcttcccctgagcccctggactcttgactccagcagggcctgggtaatgaacgtcagctcccctttcccaaaggggttgctctgttgggaaggcacccgtttgatacagtagcatagagatgggttttagcatcaaaatatcagaattcaagccttgctctctgcttactagctgtgtgaccctaaaaaggtttctgaacgtctctgagcttcagtttcctcatcattccttctcacggggtggttgtgagcattacagagatcctctctgtgaagcccctgtgagtggctcatcctgagggctgaaataaacatgttattaataatccaaaactggcaagggatgttgactggtccccctcccttgcccaaggagctttctagaacctgagttatcattaccaaactgtactgccttgagtaagaaagttagaaggaatgggaaggatggtggcaggtggaggaaggcggattggtcatcacctccttgcagcaagaaacagccccagatcgtgggaaacctacagacctgctagacagactaggagcaaaagctggggctttaagaatccccagggaggttctcctgagagagtagccagttggattttgtaagcagagatttgtttggggaggaggtgacaacgtagggagcagaggggcaaagctgtcgggaatcctgccttgagggcagggatgtgtgttggggggagttgggtcactggggctcggtggccttgggcaagtttctacctctcaggtcctttacccacctagggtcgccatcctgcccacctcacaggttacagtgagcctggatgcactgtcatgggcaggtgcccaggaaaatggcagacatgttccaaacagcacgcagcattccccagtgatgcccagggtcaccttggaggtgggcgagatgcctggggtttctcgtccaccccacaacacctcaggggacagccaaagctgtcccttcaggtaagctgcacagaagatgtgaactctgctgcaaagactctattctttgggagcaaaagggacccagggtctcacctgcacatccctgtccctgagggcctaggggttcttggaggccccagccttggcaaaatgaggaagaaggtgaaggttgtctgggcccctgccaggctccttcctcggccacgcactccccttcctgcacacacacccttctccctccaccccatctccattgttgtcagaaaagtcacaataaaaaggtccatattgtctagttcccatacttttaatttttaaaattttatttatttatttatttatgtattttttgagacagagtcttaacccaggctggagttcagtggcatgatctaggctcactgcaacctctccctcctgggttcaagtgattctcatgcctcagcctcccgagtagctgagattacagatatgtgccactatgcccagctaatttttgtatttttagtagagacggggtttcaccatgttggccaggctggtctcgaactcctggcctcaagtgatctgcctgcctgagcctccggaagtgctgggatttcaggtgtgagccaccgcactcggctccacacttttcacttattaaaagactgtggtgtccatcaatggatgaatgaataaaccaatgtggactatccctcccattacccaaggaatgaagcacggagccgtgccaagatctggattcacagtgaaagaagccagtcaccaaaagccacgtgctgtgtgacttcccttatacgaaatatccagaagagatacatccatggtgacagaaagtagatgagcagctggggactggcgaaggggagaagggggagcagctgtctatgaggtccagcctttcttctgggtttggtgagaatgttttggaactagatagaggtgatagttgtacaacattgtgaatgtactaaatgccactgaatcattcattttaaatcgttctttacgttgcatgaattttaagtcaatcaaaaacagttgtttgaaaagagaaaagcctatgggtagcggcagcagtgattggatttatgattcgattccatggctcatccctcccctgcctcaccccctcgccctccgacgtcttcttcttttactctgaactgttatctttgttctcatctctctctctctctctcaaccctgcagacacttttccctttctttgtctgcccccaccctccagatttccgtgtctccagtgtctccctacgaggcatgaattgagactgggagggtgtgattctgaagaaggcaccaacagtgactcagctagccccttcccccaccccgccccccgggcctcaatttagctaaaaaaccacagggacggactcaggaggcaatacctttccaagggtccctaaaaaatgtcccattttagtgtccaggtttcactcaactttagtgcctcccctaaaatgtgttccttacctcccaccccactgcatctaagtcactgcctgagaaaacaggattgaggaaaggagaaaggaagagagagagagaggaggagagagagagagagggaggaaggctgatggatttagaaaagaagaaaacaagtggtctgaggaaaacagccttggtgtgtttattttcctgtctgtgtatcgcttctcggccttttggctaagatcaagtgtattttcctgtctgtgtgtctcgcttagattacagggatctgtgggtgatgacacgtctggtccaggctgcgtagtcacctcaagggcatgcttattgatgtgtttttcaattcactatctttgcatgggagtcccaggccaagaggcacagctgcgccatttgtctgttggtttagatatcctttatccagttcttccagagaaatcatcctgcccttctggaggaggtgggcagcaggggtcagagatgggagggaaaggaaggagccaggtccttggctaggatgccagggtcccctgcctctcacctggcctgggctggaggcctcctgctgtcctgtcactgatcactaccccgccccagcctcctgagttagaagacacaggctaaagtagagtatttcttcattgaaaaacccatacaaaataaaggttcataaaaaataaaaatttagactgggtgctgtggctcacacctgtgatcccagcactttgggaggccaaggcaggtggatcgcttgagccctggggttcatgaccagcctgggcaacatagtgaaaccccatctctacaaaaaatacaaaaaattagccaggcatggtggtgcatacctgtggtcccagcttctcagcctatggacccacatagaatacaatgtcagcataagaagggagccctggggtcaccaaatggtttgggcggcaaagaacctgaaggttgagagaagtggcttggttacccagctgttggatgtgagacctggccactgcttcttccataccctagacctgcaccctgacatctcaagtaaaaagttgggggatgttttatggtccaggatgaaggaagggcagtgaggggcagcggagcatcactttgcatttctgtctgcctcttactggctgtgtgacctggggcaggtaacttcccagactcctgggaatcataacacctatgatgatgatgatgatgatgatgatgatgacacctacctcaaggattgccctgaagggtcacagagatgcctgcaaggcacctgcatggagcaagcgccccttctctggcaggtgctgggtgagcactacctgctgccaggccctggggctatggcactgcgtgaccctgcaagtcctacctggcgaagctgtcgttcttgtgctcagtcagtgttggttgtaagactgagaagagtcacttcattttgctctccagggacatctttctgggtcctattttctgcctatgtcaagtagcgcctcaaggatgctcctgaaaatgggcttgtctttcttaacatggcaggtaggtcccaaagcattagcatggggcagctgacctagcccagccaatgcagtgcagtgactcttgcaaccgagtctaatcagaaggtccatgaacctacgagcatttcctgtcccaggatcagggtggaggctgagcctccctgcttagagattcttcccatgcattccacttttttccccaaaagaaaatattgacccttgagaggcacacagtttatttattttgcatagtaaatagtagcctgtattttaaggatgagttgatttctgcatcagcccctgtaggtcatcagccttctattggtgcatctgactctctctagccctgcagggatggtggagggggaggggaaggagggatctttattggaaaccaggacagtgagactcattgccctgtcatctgctctgtggtgctgaatgaggcagcccaacagagaaataccctgagcgagcatccccagcctccaaaacagtggcgcattgccctgagtcctgggaatgacctttgattctcctgctcctgacttggaacccatggaaacctctagaagcagctgaggaaaacccaacatgaaaagcagaactccacactgagaatataggaggtgatcggaacatacaatgattcttgctaagaccgattcacagtttttcttttttttcgatcgaagaaatactggagaagcctaaagaaggagtctaaaaactctggcacgtgggccaaaactgtccttgagctaagaatgattttcacatttttaagtggttgaaaaatgaaataaaataagatgatgttttgtgacacatgaaagctatgggaaattcaaattctaatatctataaatagtgttttatcagaacacagtcatgctcatttatttatgctcgatggctgctttcccgctacaattacgttgagcagttacaacagagaccacgtggcccacaaagccttacaatatttactatctggccctttccagaaaaaaatgtgccgactcttgaccttaacctcagcaatttgggaggccgaggcaggcggatcgcttgagctctggagttcatgaccagcctgggcaacatagtaagactccatctctacaaaaaatacaaaacattagccaggcatggtggtgcacacctgtggtcctagccactcgggagactgaggtgggaggatcgcctgagcccaggaagtcgaggctgcagtgagctgtgatggcaccactgcacctcagcctgggcgacagagcaagaccttgtctccaaataaataaataatgcaaagtaaaataaataaaaccatataaaaaggaatcaatttaaaattataatgaaagctggccgggcatggtggctcacgcctgtaatcccagcactttgggaggctgaggtgggtggatcacgaggccaggagatcgagaccatcttggctaacacggtgaaaccccgtctctactaaaaatacaaaaaaaaaattagccgggcacagtggcgggcgcctgtagtcccagctactcgggaggctgaggcaggagaatgtcttgaacccgggaggtggagcttgcagtgagccgagatcgtgccacttgcagtccagcctgggcgaaagagcgagactccgtctcaaaaacaaaaacaaaaacaaaaacaaaaaaaaattataatgaaagccaaggggcatagtagaacaaattttctagagctcattaagtcaaatgagtcaccagttagtaaaacgcagtcacggggaagagagggcaggattctttgaagcagcggctctcctaaaaacaacccacccttgtccagctgccttccctcctgagggtgttccctttgactgtgtgacccccatcccctatttcccaaccgtccaagcccacctctagcataatacgagcttttaatccctctccctgaccccaacccgattttgaagcccagtctagtattttctcaaatacacttcttggctccattccttcctttccatcacctctgccttttcactgcatgcttggaccactgcagtcagctccctatgaacagttgctctctacccatccaatcggccccgcctgctgctgccaaattcaccgagggcacctctgtggtgctgcctgtggacaaagtccaagccagccacctcacccacctacaggtgagtggggagcagccagcgtgtccagtggtttaccccatcgccacagacttggtgatgtgtcgatgtgcagagaaggggtgttggcagccacaacacaagcaaccccgccccatgtgagatctaagatgggcgtgctgggagccacctctgagaatccaacagaaggcagaggggagaacggctcacacggcacaaacactccttccttttttttttttctttttcctttttgaaaggagtctcactctattgcccaggcaggagtgcagtggtgcaatctcagctcactgcaacctccgcctcctaggttcaagcgattctccagcctcagcttcccaagtagctgggattacaggtacactccaccatgcccggctaatttttgtgtttttagtagagacggggtttccctatgttggccaggctggtcttgagctcctgacctcaggtgatctgcctgccttggcctcccaaagtgctgggattacaggtgtgagccatggggcctagcctccttccatttaaatgtatgcctaatttgcccattgagaacggctgagacgcattttaagtggccagggtctacttagagttagtgctcatgaccaggcccaggtcaagcctggctggccagatggtgcctttgacctgctctgtctctgtgcaaaggaatgagctgaaggatgggggtgcagtgtgtgggcagtgggctggggctggcaggactcagtgactaagggaagagaactttcctcactaccagcctgtcttttcagggcaccgcggggggctttgggacttggtgatgaacacagcacagagagctgtccagcatgcgggtccctggcttctcacacttcccaggctccttcagaggctctctccaaagggagctgctctctctagaacccatgaatttggaatataggcaaccactgcattggggaccactgacctcaaacatagagaccagagcaaatggggctcatcacgtgaaactcatctggaactctagcaggttcttttatatatatatatatatatatatatatatatatatatatatatatatatatattttttattattatactttaagttctagggtacatgtgcacaacatgcaggtttgttacatatgtatacatgtgccatgttggtgtgctgcacccattaattcatcatttacattaggtatatctcctaatgctatccctccccactccccccaccccacaacaggccccagtgtgtgatgttccccttcctgtgtccaagtgttctcattgttcaattcccacctacgagtgagaacatgctgtgtttggtttttttgtccttgcgatagtttgctgagaatgatggtttccagcttcatccatgtccctacaaaggacatgaactcatcattttttatggctgcatagtattccatggtgtatatgtgccacattttcttaatccagtctatcattgttggacatttgggttggttccaagtctttgctattgtgaatagtgccgcaataaacatacgtgtgcatgtgtctttataacagcatgatttatattcctttggttatatacccagtaatgagatggctgggtcaaatggtatttctagttctagatccctgaggaatcgccacactgtcttccacaatggttgaactagtttacagtcctaccaacagtgtaaaagtgttcctatttctccacatcctctccagcagctgttgtttcctgactttttaatgatcgccattctaactggtgtgagatgttatctcatggtggttttgatttgcatttctctgatggccagtgatgatgagcattttttcacgtgtctgttggcgaactctagcagcttcttttcacaagttcatggagagaggtttcccactgagggaatcacatctgtctgatcaaaagaggcttgggaaatggctctcctgttcattccctgaaaacctctgatggaaccactgccactgtggcagccccagcactggcaccccagccatgattggtgccccagccacatctctgctgtgagccccagagccctggttaattaatcatccacgtgttgatggggagaggcccattcacaaaagcgacataaagcccagggagacgtggccgtggcaagaagggtgtgggactacattccgcccccaactgagagattcagaaaccagaaaaaaatggaaaaacatactgtgctcttgggtgggaaaactaaatatcatgaagggagcaatttttatagttttggcctataatacaattccagccgaaatcccagtggaactttgagaatttgcaggaaaaaaaaaaatgtctaaagtacatctggaagacaaacttacaagaaggtcaaataattttgaaaaagaaaatgatatctaagcccacctagagaataagacttgagatccaaagctaaatcaggaggctctagcaaaattgacagataagcaggacagagtgcatggtgcattcacctggggaagagggcagattggtctacaaataggcctgggtccactgactttagctgttatatttggggagaaacttttcaacctcactccatcttaaacctaaaaatattccagatgaattaataaatataaaaaattagaccactaaaaatgtagaagaaaatggatgatctttctataccatagagcaatggaataaatcacaaaggaaaacagatttgactatataaaacttaaaccctgcccatcaaaaaccatcagaaaccaaaataaaaggcaaccaactggagaagatagttgccacaaatatgatcaagggttaatgttattcataaattaagagcccacacaagtcattagaataagcactgagacctgaacagacaagcaaaaagaatgagagtgggtcggcgcggcggctcatgcctgtaatcccagcactttggaaggctgaagcaggcggatcacttgatcccaggagttccaacaccagcctgagcaacatggtgaaaccctgcctctacaaaagtcataaatattagccgggtgtgatggcacacgcctgtagtcccagctactcaggaggctgaggtgggtggatcacttgagcccgggaggtagagtctgcagtgagccaagatcacaccgctgcactccagctggagcaacagagtgagaccctgacttaaaagaaaaaaaaaaaaaaagaggagaaaaatgctgatctcactagtaattaaaacatcaggccaggcgcagtggctcacacctttaatcccagcactctgggaggctgaggcaggcagatcacttgagatcaggagttctagaccagcttggccaacatggtgaaatcccgtctctacaaaaaatacaaaaattcgccaagcgtggtggcacatgcctgtgatcccagctactcgggaggctgagacaggagaattgcttgaacacgggaggcagaggttgcagtaagctgagatcgtaccattccagtccagcctgggctacagagcgagactctgtcccagaaaaaattaaaacatcacatatttaaacaactctaggatatcatttaaaaaaacattaatagactgttttttagagcacttttaggttcacagtgaaactgagtggaaggtacagagacttcccgtatgttccctgccctccacgtacagcctcccccactgccaacgtcctgcaccagagtggtacacttgttacaaccaatgaatcctcattaacatatcattatcacccaagttcatagtttacattagtaaaacatcatctttcatctataagcacaaaaattttttggcatttatttaggtgtatgattaactcagtgttgacaagactcacacttcatacccacttgcactgcatctgagaagcaattggtgtctacagccgctacaccctcaacaagcccgatcttgtttgaaaagcaattggtgatgcttctcaaaattctatggacaaagtcagccgggcatggtggctcatgcctgtaatccctaaactttgggaggccgaggcaggcagatcacctgaggtctggtgaaaccctgtctctactaaaaatgcaaaaattacccaggcatggtggctggggcctgtaatcccagctactcgggaggctgaggcaggagaatcgcttgaagcaaggaggcggaggtttcagtgagccaagattgcaccactgcactccagcctgggtgacaagagtgaaactccatctaaaaaaaaaaaattatggacaaagtttttcaaaaagatatttaatgcaactttatttgtaatattggaacatctgaggccatttcagtgctaactattaggggatggttaggaaaatatggtacatatgtggaaaggaacatttggtagttagtgcccctgatgtttacaaaggcttttagtgaccaacaaatgctcatgctataatcttatgtgaaaaaagcaagtagcataattgcaactatatttttaatgcatagaataaaaggctagaaggaaatatcacagatccttgacatacattcccaaacctttgtaaatccgcggattcatgaaaacagacacatttgcacaagtgcctgatcttttctgttatacattcattagaagtcaagccctggtgccacaaagtatctgccttttcaaatgtgatcagaatgttctcttttgcttcaaggccatttttcacgaagcagtggcatttttgcctcttcatcagagtcaccgtgtgccctggaggactgagaacagcagagccgttttaggatgggacagggcagccaggaggattgggctcactccctactgagtgcctcactcccgtacagcccccatagaggaagaggggttcaaatttattcctcagccagatggcatgtgccgcctgtcctggaatttcacatcacttatgatggaccaaaattccaaaagctgaatccatgattgtcaaagtctggtatggcaggatgtcaacagtaatcgtttctgggcagagggatgattttctcttcccatcttgctttgtataaatacattttctataataaggttgtattacttttctcatcaagaaatagcaaagtactgttttactcaaaatatgaatagagccaggcatggtggcagcttatgcctgtaatcccaacactttgagaggcggatatgggaggatcactttagcccaggagtttgagaccagcctgggcaacatagtgagacccccgtccccactcccccaaagaaaacccacaaagcatttatcctggattattcacaggggccaaaaaaaaaaaaaaaaattcaggcctcctatagccatgagctacgaatatgaaaatatgcaaatgtgtaagaaaagccagcacatccgatttttacttttactttcacacctctgtccaccatgttccaagagaagaaacttggtcattgaaaggaatagatcaaatccaaagaacaaaaccactgtgctcattaaacttcttagtgttcacaaagctttagctgcaggttgaatggggcaacccgaattggctggctcacctgggctgcagggagcagagatcgcgacactgcactccagcctgggcaacaaagcgagactctatctcaaaaaaaaaaaagttcataaattcaaagttatgaattatttttaaaataataataatttacaataaagatgaggacaaagtgtgagtaaatggtggtttctatccagctctgttgagctgaagtggcatctccctgctggggcttttggggaagaagggtgtgtgttgctcttcagatcccaagcctcatgcccctactgggccctgtggggtgcttctcagcccaccaggagagccaccgttggaacacacacgtgggggacctggtgggtgccggtgtggtgaatgggggccacagcctgactccaggaagccagcaaactcggagctggaggagtcaggacacccccgatgagtcaagagttggttttgctgccagttgacatctgattgaaccatctcttcacttctccgtgcctcactttccttaccagacaggctctgctgatgctgtccctctcctgttcagtcgtgccctcaccgttaaagagaaagagcaaactgctgggcagcagcattgatttttttaatgaagtggaaagagagctgggaataacaagtcgggcccacctcacctgcctcacctggtgggtttatttgttttgtttttttttttttgttttgagacagagtttcaccctgtcacccaggctggagtgcagtggtgtaatctcagctcactgcaacctccacctgccaggttcaattgattctcctgcctcagcctccccagtagctgggattacaggcacctgccacatgcctggctaattattgtatttttagtagagatggggttttaccatgttggccaggctggtctcgatcccctgacctcaggtgatccacccacctcggcctcccaaagtgctgagatcacaggcgtgagccaccatgcctggccgtcacctggtggtgttgaatatgaactgctgcggtgttggtaaattaagcaagcagatagatgtaaataacgcttgggcaggaatatggagcacgggatgaggatgggcggccaactgttagagagggtagcagggaggctgagatctgcctgccatgaactgggaggagaggctcctctctctcttcacccccactctgccccccaacactcctcagaacttatcctctcctcttctttccccaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaaggttagtggacagccatgcacagcaggcccagatcactgcaagccaaggggtggcgggaacagtttgcatccagaattgcaaagaaattttaaatacattattgtcttagactgtcagtaaagtaaagcctcattaatttgagtgggccaagataactcaagcagtgagataatggccagacacggtggctcacgcctgtaatcccagcactttggaaggcccaggcaggaggatcccttgaggccaggaatttgagaccggcctgggcaacatagcaagaccccgtctctaaaataatttaaaaattagccaggtgttgtggtgcatgtctatagtcctagctactcaggatgctgaggcagaaggatcacttgagcccaggagttcaaggttgcagtaagctgtgattataaaactgcactccagcctgagcaacagagcaagaccctgtcaaaaaaaaaagaaaagaaaaaagaaagaaagaaatttaccttgagttacccacatgagtgaatgtagggacagagattttagggccttaacaatctctcaaatacagggtactttttgaggcattagccacacctgttagcttataaatcagtggtattgattagcatgtaaaatatgtgactttaaacattgctttttatctcttacttagatcaggcctgagtggcctctctttagcaagagttggttagccctgggattcttactgtagccacattaataaacaacatcgacttctaaacattctataataccatcttttggccaaattgacttcgcctcttcctctctctttccaaatgaaatgtgtttcatttcactgtcagaccacatggttggggaccccacagagcacacagccctccctctgccttcccatgctggcccttcacccactgctggagtgccaggttggtccaagggttggaccaagttgtctgaggttgtctcaaggttggtcgaggctgtctccgcgctgggttgtgctacaaggagcccttctttccatgggtgtggctggcagtgagtgctcacagcaacagcccacagtgcagcccgagggcaggatggactcagtccctgcctccatacccatttctaaggaggcaaaatggcaaacactctacttttctcttttaatgctaaaaataagaaaacaccttgcagcccagggtatgggtagtgcatggaagccgtggagttgtgaggtgggaagtgacctctgctggatatgtctattcaggaagattgctggagtgggtggggtctctgggaggtcccctgagtgtgggaagctgggaccaccagctttctcgcacagggagtggccatcccagcttggagaggttccaggactggttgggaggcacgtttcagatttctatctgttgaatcagcgaagatattggattatgaggaatttgggaattaggaaagtgggtgcaggtgggttgggggtaggtgaaggaagacatgggcgtattgggggagcaggggctgctcagaggtgttccagaagctctgggtgaggaggtgagagggaccggggaatgcagctcggcccagcctccctgcctgaggtcagccatcacgtggtgatggcaagatggaaatgtgctttctgactgctccagccagtgctgccagattcagctccccagggagggcacctgagaggctccaagccaggagatctgttttctcctttgttttgtttttttttgttttgttttgttttattatactttaagttctagggtacatgtgcacaacgtgcaggtttgttacatatgtatacatgtgccatgttggtgtgctgcacccatcaacttgtcatttacattaggtatatctcctaatgctatccctcccccctccccccaccccctgttttctcctttgaatccttcttagaggccgggcgcggtggctcacgcctgtaatcccagcactttgggaggctgcggcaggaggattgcttgagcccaggagttccagaccagcctgggcaacatagtgagacctcgtctctacagataataattttaaaaattatccgggcatagtggcatgcacctatagtcccagctactcaagaggcagaggcaggaggatcacttgagcccaggaggcggaggttgccgtgagccaagatcccaccactgcactccagcctgggcgacagagacccccatgtcaaataataataataataaataaatccttctcagtcccttcctcactgtgtccccctccactgaatttttccacctcctctcccacttcccccactcccgctttccctctccttctctccccactccatctttttctttctctgctgtttctcgtccctccctcctctccatcccacaacactgcctaccctgtccctgccccaccctggtgctcaggatgtgtgaagtgaggggtggtagcccccaagacctcaaccccgaaggttagcctgttgaaaccactttctcccagctgcccccctggcagttggtgctgctgggggaaactgggattgggggccagattttgcctcttttcctgacaaagagagatgaagagttctctcaccaggtgcctgggactggggtgtgggtgtcccagcctatcccagcgcatctgttctgcatcatgattaatagtgctgctttcagccgggcgcggtggctcacacctgtaatcccagcactttgggaggctaaggtgggcagatcacaaggtcaggagttcgagaccagcctggccaacatggtgaaacctcgtctctactaaaaatacaaaaattaaccaggtgtggtggtgggtgcctgtagtcccagctacttgggaggctgaggcaggagaatcacttgaatctgggaagcagaggttgcagtgagccaagatcgtgccactgcactccagcctgggtgacagagtgagactccgtcctaaaaaaaaaggagttttgctctgtcgcccaggctggagtgtagtggcgccatctcggctcaccgcaacctgcgcctcccgggtgcaagcgattctcctgcctcagcctcccaagtagctaggattacaggcgcctaccaccacgcctggccagttcttgtatttttagaagagacggggtttcaccctgttggccaggctcgtctgggactcctgacctcaggtaatccgcccacctcagcctcccaaagtgctgggattgcaggcatgagccaccgtgcccagtcaactccttctcaaaaaaaaaaaaatagtgctgctttctctttcaagtgtcctgatttgggtgatagtaaatgccactctacttataagggatctacctcagaatgctaattgggacatttttgtagcactctactgttggcagcaggtgatgctcacaacagcccgtgagggtggatgacgtccgcttcacagatgacaaaggagcctcatgctcagaccgtgggctgccagagcaggtccatggctgcagccccacatggaccatatttcccccttgtcactctttccaccaagctcccttggaacttcagttattaagctctcttgggtggaatccaagttagaatcacaacatgtgcctcatatggattgtgccagtgaaaaatgacattctatttagaggcagggcagcctggcttagagtcagtttaaaatatgtattatgctgcaacaaatgtaccatgatcctgtaagatgttcacaacaagggaactggatgtggggtatactgtctgtactaacttcacaagttttctgtaaatctaaaactgttccaaaataacaagttcgtttaaaattaactccaggagaccaggtacggtagctaatgcctataatcccagcacttcggaaggctgaggcaggtggattgcttgagcccaggagtttgagacaagcctgggcaacatggtgaaatcctgtctctaaaaaaaatcacaaaaattagccaggtgtggtggcgcattcctgtagtcccagctacttgcggggctgaggtgggagaatcatctgagcccaggagtttgaggctgcagtgagctgtgattgtaccactgcactccaacctgggcaacagagcaagaccctgtctcaaaaaacaaaaatgaaataaagtccaggaaagaagtaggttttaccactcttattttctgaagagaaaactaaatttaatgtgtaaagtgaggacaagttcaccaagttagtgtttgagttgcctaaaatatgtttgctaaaactattcaaagctttcacataaaacatgatcagaagttctatgccaaaacatatgtgtgtgtatatatatatgcactatatatactgtatataaaaatgcaaaatctaaattgccaaccttttagaaattgctctgaaaggaaagcatttcaagataatttgcttacccaaagaatatactttccaagaaagcaagtaatacttaaggtgttcataatcctcatcaaattaattcttgctactgaaagcttacaaggagctgttttgatgtcgggtgtgacaggtttgacttggcagaaggtgtcactttactaacaacattttaaataagtgacagaagacaagaaactacacgttaaatgccagaacaaagagtgtctaagtggatgctaagagttgaaatatggctggatacctgcccaagagagctgaaaagtagatgaaagttggttacctataaactagtgcaccctaatgaattaaaaggtgttgatgagttaacttgttatgccttccagataagacatgcaaatggggcttcttcctccttcactacttccaagggatttaacaaggagaccaatgcaaatgataaggactgtagggctcaagctggggacagattggggaaagggggaccatcatgcccatatagatgtccctgtgccctggcagtcaaggctgctgaaaaataacaaaacccagaagtctgcgtgatgctgcctctccatttgtccaaagccttcttgcggcagtttgcaggcttttgcaaaagctccaggaccaaggagctatgttcatgctggaagcttgttcaggattagctgttctttgtgggatgggtgcagccagggccaggtgtccagggacagtgttttaacaaagggcatgaggtgtctgatctcacagtggaactccacttgcctttttttcatcttctcattctgcttcatgcacagaaccagccccatcctgaaactgactctaaattactcccgccccaggtggagtgcctttctcggagttcaacagagccttcctgtcgcccaagggacaactccactgaatgcccaagccacacccaaaacctaacaagtaaaaaccaaattctgtgctcccccatcctgggccattcctggtttctctactgctgttggtgataccaccatcagcttgtccatcatgaccctggccagttcctcccacaaccctccacagcacccagggacctcacctccattccatccgacacagatctcctcaccacaaaccttggttttgcaacagcagccatgagacctttacaccctccgcccttcatcctgtcccccactgaggccccagagccattccttaaagcagcgcgccacaaactataacccacaagccaattctggtacccagcctgttttgcacagccagtgaactgacaatgatcttttcatacagccagaaaaacaaaacaaaacaaaaaacaacaaaaaaaaaccccaccattctgagcatgtgacttccatgttcaagatgtctcatgttcagaaaggcccctggaaaaggaggaaggggagctgggcacaaagggagaccctctcagctgagctcctcccatccagacattttcctggacttcctatccaatgacttcccttagcttcttatcagccacccctgtctgcccaggaggctggaagatgtggccttttaactgggcacagctctgtcctctatcatatcagggctctgttcccaaggagggtagagagaatggacaccaggtggaccctcagcagtctgtgccacagagggagtgtttgcaatttccagactaaaagtccccatgtgcttgacggggtatgtgactacaacgtgatgcttgacttttcctcatatgaccagagccactttgtccatctggtacaatgtcagctatctgctaggggccctccaggattcccagtcaattccatatctgcatcaccaccattggcactaaataaaataaaatactcaagttcctgctggtgagcatgagcagtgctacactgggcccttcaaccaaggtgacatgataatgactgaaaataatcactgccacttattggggacgtctcatctgccaggcatggtacaaagtgctttaaataagcattcaacaatttcatgctgacagaagccctgtgagccagtggagctactactatgcccattatacaggggagaaaactgaggcagagagaggttaggtaattcgctcagcctcacacaaccaataggtggtggagccaggatttgggccccatctgcctgactctctagaggctctatcttccagtcttccagagttgagtctaagccatgaataggacaattagacagcagaggaaacccattcagccaccatgtgcatgaagagtaaggaatttctgtcatacagaggggagtgaattcactgagctgagagctgaggaaccattgatctgatggctgagacaccactgggaagactggagaggcttttctgggcatgcagtgccaggcacaggaggagctgagggaagatgactaagaggtactggcaaagaattcagaaattctgatggaagctttacatgttaccatcacatccatccatctatccacccatccatccacccatatcttcctccctccacccaatcatgcatacatccagtcatctatacaccacccacccacccatccatccatccatccatcccttcatccatcccatcatccatccaattatacatacatccaatcatatatctgtacataatccattcttccctcggttcatccatccatccattcatccatccatccacccatcccttccttcatccttcctatcatccatccaatcatatatctgtacataatccattcttccctcggttcatccatccatccattcatccatccatccacccatcccttccttcatccttcctatcatccatccaatcatacatatatccaatcatacatctgcacatcaccagctcatccatctatccatttatccatccatccttccttccatccatcattcatccatcatacatacatctaaccatacatctctacatcattcattcttccatcgattcatccaattatccatcattccttcctccatccatcccattatccatttgatcatacatatatcatctatacatcatccattcatccatccatccatccatccacccatatcttcatccaatcaatcatacatacatcgaatcatctacacatcacccatccatccatccatccattcatctatccacccatccatccatccatccatccattcatctatccacccatccatccatccatccatccatccatccatgtaaccatccagtcatatatccaattacacatccatccagttatacattcatacatgcatctaatcattcaattatacatacacacatccatataattctacatccaattatacctccatccaattacacattcatacacccacctaataaattattaattcatatatccatccatataattatacatcaattatacatccatctaatcattcagtaattcacccaccatccagtcatctatccaataatacattcatccaatcatccatccatccatccacccattcatccatccatccgtccgtccacccatcatggtatgagccatgatttaccacgatggtcccctgtggacagcccaggtggggcagaactgaagggaagcccagggctgcccccataaacatttgcctcctttacatggatgagaactagatccacatgtataaatcctcatgatttgaaggtgcttttaccaacattcactcatgggattctcccaggagctctaggaggaggcaggtagagttgaggtcatctcacgcattttacagatgaggaaacggaggccctgagaggcaggtccaaggccacctgaccagaaagaagtggaactgggacttgaacccagccatcttgccccttggtcccatgctctctagcctgtaactcctgcttcctggtggggcatctccaggaggaccctatcggctggccatgggcctgccctggagtcttttgctctgtgtggccatccttcctccctcaggagagtgtgtgctcccagagcacaggctgtatcttctgagcattttgtcccttcccagtacctagcactcagctctgtatacattgggctctcaagaattctcaaccttccagagtgtaaggccttgacctgctcagccctggatactgcatgatgcattgataagcccataaaataaccagggcagattgactcccagtggccaaagtgccacagggaagggacaattcagcccttctaggaggaggaggaggtagttttctcatttctattaaggcaacaaaagctgccttactaaggacattcttggtggagggcgtgactgtcaaccactgtgatcatttgggcctctcttgcccaggcttcccattctgaaaggacagttttattgtaggtacacatggctgccatttcaaatgtaactcacagcttgtccatcagtccttggaggtctttctatgaaaggagcttggtggcgtccaaacaccacccaatgtccacttagaagtaagcaccgtgtctgccctgagctgactccttttccaaggaaggggttggatcgctgagtgtttttccaggtgtctacttgttgttaattaatagcaatgacaaagcagaaggttcatgcgtagctcggctttctggtatttgctgcccgttgaccaatggaagataaacctttgcctcaggtggcaccactagctggttaagaggcactttgtcctttcacccaggagcaaacgcacatcacctgtgtcctcatctgatggccctggtgtggggcacagtcgtgttggcagggagggaggtggggttggtcccctttgtgggtttgttgcgaggccgtgttccagctgtttccacagggagcgattttcagctccacaggacactgctccccagttcctcctgagaacaaaagggggcgctggggagaggccaccgttctgagggctcactgtatgtgttccagaatctcccctgcagacccccactgaggacggatctgaggaaccgggctctgaaacctctgatgctaagagcactccaacagcggaaggtgggccccccttcagacgccccctccatgcctccagcctgtgcttagccgtgctttgagcctccctcctggctgcatctgctgctccccctggctgagagatgtgctcactccttcggtgctttgcaggacagcgtggtgggagctgagccttgcgtcgatgccttgcttgctggtgctgagtgtgggcaccttcatcccgtgtgtgctctggaggcagccacccttggacagtcccgcgcacagctccacaaagccccgctccatacgattgtcctcccacacccccttcaaaagccccctcctctctctttcttcaggggccagtaggtcccagagcagccatttggctgagggaaggggcaggtcagtggacatctgatcttggtttagtatccttcattttgggggctctgggtgtggcctgggcctctggactttggccacggtgtttgttccagcccttctcctaacctgtcctttccagacactcggcatctaggttattagcacctcgcatactttctgacatgctcctcagtcctgattttgaccatcttctcttgcttcccatctgtgtcagtcaagactgcatttggctgtaagaaacagaaaccccaactaactgtggcatttacatgaagaggtttacttttctcacataatcagatgtctagacttggccagcacctcaagggtcattgatgctctcctgtctttattttctgtcatctttagtggttggattgttgcctcatggttacaaagtggctgctgcacttccaggcatcacatctgcctttgaagcaggaacaagttgcaaagtaaagtggccaaaagggccctgaaactaaatgtgtccccttaggaaagcaggagttttcttgcaagtggcaatcttctgcttatgtctcattggccagagctgggtcttacggccaccccttgctgcgagcaaggctgggacattgagcattttgccgtccaacctctttagcagaataaaccaagggggaagaacgttaatagtggcttttgagtcactagttggcagtatctgcccctctatctttccatcctccccatggagtttcaaggttcctttctcagtacttcttcaggctctgcacgttcatttggatcttgtgtcttggggtgaaaaactggcccaagtgtctccccaagcatccacctttggattaatttggaaaatggctgtcaagtgcccgcctcttgcttggtataatgctacagctttagaggacgcagcaggcatgggccttgccgctgaggttcttagcctcatgagaatatccagatcagattctcttggctccttcttagagccagtgatgcaagacacttcctgctcatcttgtcgggacggttttacaagttgcctgccatcctgagaaagtctacaaaacgatgccagacctcatgccagcttcccaagccttgactctcagtgctccctcaacaggattctggaagaatctcccaaacaagtcgcaatgccctctggaccctgtgcaggcatgagactcaagagcattggctcccacccctggtggagggaacactgctggggctgggatcttgcctggttgctccgcctgcacccaagacaaccataattaaaatgtccttcattgaacttggaaagccttcaaagctgacaactccttatgtgtacccggaaaggcctgggagtgtgccagggcattgctcgggagggacgctgatttggaagcatttacctgatgagagactgacagcagctcctggtagccgagctttccctcctgcctctgctgtgaaggtggacccatccaacagtcaaatgcctgactctggacaggagcggacctatttattgccatgcaagggactctgcacttttgaattgtgggtcatgggcttggatttaggggttagagctgggagaagtcttggaagtcacctagagatgacactgccattttgcagatgaggaaaccgtccaatcaaaatggaccaaggacttgcccaaagcctcacagcaaaaccataggcccccgcactaaccccagagtccctgtgctgtcttaaggatcatatagttgtaagcaatcatctggttttcagtatttcttcttttaaaatgcctggggccatgcccagcagtctgtttcactgcagcgtttacacagggctgccgggctttcctggtggatgagctgggcggttcatgagccagaaccactcagcagcatgtcagtgtgcttcctggggagctggtagcaggggctccgggccctacttcagggctgctttctggcatatggctgatcccctcctcactcctcctccctgcattgctcctgcgcaagaagcaaaggtgaggggctgggtatggctcgtcctggcccctctaaggtggatctcggtggtttctagatgtgacagcacccttagtggatgagggagctcccggcaagcaggctgccgcgcagccccacacggagatcccagaaggaaccacaggtgagggtaagccccagagacccccaggcagtcaaggccctgctgggtgccccagctgacctgtgacagaagtgagggagctttgcgtgtttatcctcctgtggggcaggaacatgggtggattctggctcctgggaatcttgggttgtgagtagctcgatgccttggtgctcagttacctccctggctgcctgccagcctctcagagcatttagggccttctggacttctagatgctcctcatcttgcctcagtcagcgcgtcagttccagagacttctctgcagggttttctggggcaggtggtggcagacccgtgccttcttgacacctgaggtcagtccaccctcctgctcagactgcccagcacagggtcacctcccaaggggtggaccccaagatcacctgagcgcacagagggtgcagatgactggaccacaccttttggtgatcttaatgaggtggtcccagaggagctcagacatgcaatctagcatccagttctgggactctgtctccttttcaaacgtattcatgtagaacaggcatgacgagaatgccttgtcaacatgggtgatggggaatcaatcagacagggcgccgggctcaaggctgcagtcacccaagagtggctcagcccaccaggccctaggaaacgcctgcacagcctggagctcctggagtcatttccttcatgtcttcttcactgcacttacgtaaagatgccagccattggtttggtgatttggagggtgcccagttgcccaacaagaaatgcagaagaggcctagccaggatttcaccagcagtggagagtagagaagatgtggccagaaaagagtttcctttccctcctaaagatggtactccctgcagctactggggaagcctgcagcattctctagggctctgtgtgttgagagcagccccaccctggccccttctgagtgcatttctgctttgtgacttgatccgtgaagtcccctgagatgggcagaggggatgtcctcgaagctggggcagagcctcatccttgaacgtgaaggacgtttgaagactgtggcatgatcacaggatgagatcacagggaacttgagtttctctcctcctctcccttcacagttatttcactgagggaaatccctcccctgcccagaatgaaaactctagccaactcttgacttttccatcactccaaagtagttgaaagtacattagtctccacagtggcaaaacagtgtgcaaaagctaaataattagaacagccagtcccatgtgacagtcaaagcttctaactccattcaaagttgcagccattcccctcgagggctggcagggaggggaggggtaagagaaacaggaaggttcttactgagttggtcctggtgtgagctgcgtcacactccctgcagaggtttcaaggagactctctctctctctgtctccatggggaccttatttgaattcttctactcttaccccagcctgccatctccagctatcctcccctgaagagcccttctgctgcgctggattctggtggccatgtcatctcctcggccccgtgggagtctgaagatctggctgcagcctcacctctgaggtcctgctagttgccacctcttaaacatgatctgaggctcccatgcactctgacctgtgcccacatggggcccacgggaaacacgctggcaagcaaactgtgggtgtgcagacggttctcagggctgcagcacctgtcctttgctctgcccccaaagcaaggccagcccatcttccatcctctagtgttccttggtggggccctgaccacagtccaccaggtccctaaccagaggggacacacaccaggtgtcctcaatgtattgccttgaaacagttgtgctgggactgtgatggggggtggccatgtagccacccccaccacccccaagccactctctccaaggaaatcctcctaaagatccctttacatcctccatgtggtggggaggttctagagttgggtgcatgtgtcttcagctactgacaatgcagaccttagttggcacctcgctctggcctatcctgtttgctgttcttggcgctccagtgaaactccccatgggccatccagttggggtgcagtgtggccacccccttgcaggttcctgccttgctggagagcacagggccctcctggctcttgtaaaacactccccatggtacagagaggccagcagtgatgtgaggcccaacctccctccatggtgttcccaagcagctccctttctggggtcaaggggtggcaaagacagtgcagcgtccaatttctgactcaagccgggcctggctatcgcagctctgcactgtgtgtgacagcaaggcaactcacccagtgccgtggcagtgaccgtgtccgaggaagcctcctcacaccctctgtctcaaggactctggcatttagctggacttgctgtagctctgagcctttctgccattgccatcaccttgtcagaaactcaggccgaatctgcactcagagttgtgcccaggcagttgagccaacacttgctcagcgatattgtcacatgacaaggcactgtcaccactgggcatcgtgggtagcgcagtgtcggctggatggacccggagggtgtctgtgtcatgctagtgctagtgatgggagccccgtgagcccattgcccgccctcccatgccctcagcagctgcctggggacagccaatggcctgggtgtttctgaggctaccacatggcttccaggaaactcgagaacctttctctcccttgcctacactcttcacacaggcctgtgctggccagcggtggggatccggcattcctatcttaggtgcagagagtgactgactcattgcaggcctgggagataagactgatggcccagccagcaagatgtatggatttctcagaggcagtggcctctgtcattgtcctcaggaaatgctggtgattctggtggcctgaggtcaatgcatgtcaacgtggccaacttgccttataaactttttttctggacaattgcgtgcactgtcctgtaacagtgtcctgttgtttatgatgcagaaataggtgtttttaaagcctattgattttggtactattaatgtggtcaggaactttctcagtctttcttgtttggggtgagctgtggcttcctaaacaggaacccaagacacccccaaaagctgctcaccagcactgccagcctccctcttaccaagtagcacccgttcaggacattctgcgaaaggcatttgcccagaagttgggaggaaggaaatgtaacattttggggcacctaccatatgccaggcaccaggctaaacgtgttcacacaaattctcttactaaccctcaccatccttctacaagacaaactagtatcttcatcttggggttcaagatgaggaaatggaggctcagagaggttgaatgaatgccggtgcctggatatgaaccccatctgcctgactccgcaacccaggcaaagtctttccttgaacttcccagcagccactgcttagacacagcctccacaaccatggctcagcagcaaattgcttctctgacctcactcagcctgtgtgtccttgttgagtgaggcattcaggaccctggtcccaaagtggagaaagtctttcctactaggtcatagctacacctgcatgtgggtgctgtgccttttgtttagtgaacttttatcaccagcatcctcagcaatgacatttgcagagaagccagagctgaggcaccttggtattcttgggatgtgactttcctgaatgtttaagggaaaatgcccgaaggtacagagagcttggtttctagtaaacaataactgtcttgcttttaccccccttcatttgctgacacatacaccagctgaagaagcaggcattggagacacccccagcctggaagacgaagctgctggtcacgtgacccaaggtcagtgaactggaattgcctgccatgacttgggggttggggggagggacatggggtgggctctgccctgaaaagatcatttggacctgagctctaattcacaagtccaggagattttagggagttggttcttatcaaaggttggctactcagatatagaaagagccctagtggtttttttctaataccatttctgggtaattcctaaggcatttagtgttctgaaagatgctagccttgtccagcctgggagttgagaatgaatgtctaacagaaactctaggccgggcctggtggctcacgcctctaatcccagcactatgggagacccaggtgggcagatcacctgaggtcaggagtttgagaccagcctggccaacatgtgaaatcctgtctcactacaaataaaaaaattagccgggtgtggtggtaggtgcctataatcccagctactcaggaggctgaggcaggacaatcgctcgaacccaggaggtggacgttgcagtgagccgagatcgcatcattgcactccagcctgggcaacaaaagcaaaactccgtctcaaaaaaaaaaaagaaactcaaatatgtgtgacaggcgattctcactgcaggctgccctgtggctgatccaggagcaaggccttaaccatgtcatccccaagcgattgcttgtaaactttcttctgtgcagccttcaacccttattatgattttcttctcaggaaccaaactgctgtattcaagaaaggcagctttgtgtaatcatttatcataaatatcttaagaaaaatcctagagattcctaattttaggaaatgggagacctatggtactgatataatgtgggctgggcttgttttctgtcatttgctagataaatgaacttgagagcctactgtaaaatgtggaagcttctagattgcagaagggctggaaagacactgttcttttctcccgagtgatgggatctgtccagtatttagagctgcctctgaggccatctgattctaggagactctgcctcgttgaggatattttgaggcctaactacacattcctgcccccagagaggtcacagcctatagcaggctgatgtttctcatgtcacatggcacagaaaggcacattttcgttctcaggctaacaaagagcttcaaaaactattagaagggacagtggctataagagaagaacctcagtcaatgtgtgaaattaactaggaacctggctcctgtttcttttaggtcatgtttttcagcttaggtaaaactagaggctttgataaagcatgacctctagaaatcattgcttttcataaatggaagtgggtttgagttttttctactgattgttagtgcaggtgatgtctacatgcccccagaacatattccatgcaacaaaaaaagcccaggtcaccgtctttgctgggaacttgacttttgtgctcactgaattttaagctttctgacagcagcctggaatcatggagggataaagtacctattagtaagatggaaaaaggtgtttcaggttggagctgcagtctgttgagagtaagctatgggaaggcctgtatacgaggggtggacttttcttctgtaagtgtccagagaccaggcctcctgaagagggcatgggggcttaacttacctggactactgtgtttacaatactcatttatcttgaactcctcctaacccctgagaattgctacatttagtatttgctgagtacttcctagcatcctagggaatcaatagaacattctcccaaccaggctgggtgcggtggctcatgtctgtaatcccagcactttgggaggccaaggtaggcagatcccttgaggccaggagtgcaagactagcctggctgacatggtgaaaccccgtctttactaaaaatacaaaagttagccaggcatggtggtacacacctgtaatcccagctacatgggaggagtaggaggcaggagaattgcttgaacctgggaggtggaggttgctgtgagccgagatcatgccactgcactccagcctgggcgacagagtgagtgagactctgtttaaaaaaaaaaaaaaaaagaacattctcctaacctggcttcttcctccaggggtgtaattaatcatgtcagtttcctcattgatacacacacacacacactacaatcctgtatccattacttttcaaggtacatttactatttacgtttggggtccttgtctcttttttaatagtgtttcttaaagtcttgtattatatcagagtacagtaacatcccagtcaagagcactctagtaagctctaggaggaaagcgacttccggaaggcagtggagacctgtcctgttggggcagcataggggcagcccctgcctctggtcagttctggcgctcaggctcagggttgcctctgggctgttcttcccagagactgacaaagggctcccataaggcacctgcagagcctgtgagaagctgaagtcaatgttttcctgacaccagttgatctgtgcaggatccattgatttaaccacctgctgtgtggcatgcactgtggtcgatgccaggaacaggaattggaggggcccatgagcatggccagtatcacaggctggaggtgctgctgcgctctgaccgggcctcttggggatgagcccatgtcaaccaccttgcctccgatggggtcgggcccacaggttacctttgtgtgtccatgaccacaccttcctccccgacctcatccaaatctctttcttttccaagcccctgaatccttcagggctgcaggttttgtttaaagcagagctggtgagttgcataggttgttgcattgggactagatggggtgttcaaagagttgggagttaaaaaacataaagggtatttattaggagaaccaaggagtgtaattctcctgttcttaatatgcggccaggttaatgaatgtcacgtgaatgaaccagaaaaaaatgaagtgtgcccttgatcagctgggttggtgtgcagcaagctgtgtgaccaggggacagcagtggtcctgagggccgtcactgtctgccgtgcagagcccttcctcccacgggggcctacctcacctgtgccaagggcttgtctgtggtcagtgacctggatagatctgaatggggcttctttttcgaggagtcttatggcaggtctctcagtaaagactccattcttgatgatcacacattttggattttccaaatctgtcagagaatgggcttgaggcggggtttgtgggcactagtttcactggtttcatttaccaaaaaggggagcagaagtcaagtatggtggctcatccctgtaatcccagaggcaagagaattgcttgagcccaggagttcgagaccagcctgagcaacataaggagaccccgtctccacaaaaatgaaaaataacattttagtcagacgtggtggcatgcatctgtggtcccagctgcttgggagggtgagatgggagggttgtttgagccctggagttaaagttgcaatgagctgtgattgcaccactgcactctagcctgggtgacagaacgagaccctgtctcaaaaaaaaaaaaaaagaaagaaagaaaggaaaaaaaaaactcatgcctgtaatcccagcactttggggaccggggtgggcagatcacgaggtcaggagatcaagactatcctggccaacatggtgaaaccccgtttctactaaaaatacaaaaattagccaggtgtggtggcacgtgcctgtaatcccagttactcgggaggctgaggcaggagaatcgcttgaaccagggagtcagaggttgcagtgagctgagatcgtgccactgtactccagcctgggcgacagagtgagactctgtctcaaaccaaaaaaaaggggtggggggcgggggcaggagaacagtgagaggtagggagaggaaaggggattctcgctacacccaaaccagataccatctagaggctagaatctttgggaggctcaaattccctagaaagcaggagaagcttctgtagccctcccgctttcccagtagattaagcccagggcggctccagatgtgtgacatgctctgtgcccaaccagagcccatcataggcagaggaataacacccacaccagaagggccctcggaggtcaccacgtccaagaaccctctttacagatgaggaaactgaggcccagagaggggagagccacctagcgagctggtggcggctagaccaggagagctgtcattccaagcaagcaaaggcaacgagacgagcccagagctgtgctcccatctctttgttagggggcctgggatgccctctcagtgtcattttgtccaggatgatgctccctctcttaagcgattaatgcgcccttgctaaccttttgctatcgctgcctcttcaaaccagaggagttgagagttccgggccggcagaggaaggcgcctgaaaggcccctggccaatgagattagcgcccacgtccagcctggaccctgcggagaggcctctggggtctctgggccgtgcctcggggagaaagagccagaagctcccgtcccgctgaccgcgagccttcctcagcaccgtcccgtttgcccagcgcctcctccaacaggaggccctcaggagccctccctggagtggggacaaaaaggcggggactgggccgagaagggtccggcctttccgaagcccgccaccactgcgtatctccacacagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaaggtctgtgtcttgagcttcttcgctccttccctggggacctcccaggcctcccaggctgcgggcactgccactgagcttccaggcctcccgactcctgctgcttctgacgttcctaggacgccactaaatcgacacctgggtgcagctgctccactccctcggcctcctcccgtgctcaggctgtggccgcacgcgcccctcacgcttgcccgccactctgcatgtcaccagcacccccgctccgtgctacccaccttgtttgactctctggccacttgatttgtccacaacggcccatcagcccacaggaggtttggtgggtgccttccaccgacaggatgacgggtgccctcatggtgtctagaactctccaaccctcccatgtaggcataagcagccccactttgcagatgaggaaacggaggctcagagaagtacagtaacttgccgaaggccaatgagtagtaagtgacagagccaggtttgggatccaggtaggttgtctctgaaagacacgcctgtcctgcatcccacaacgcctcccaggaggtgctggagtgtggacgcctaacacagagatgtgcagggcacacacagcaggtgacacacacagcatccagaggtggcccagagctcatgctgtgcctttggcccagtgccctgcccccacccactctgccttgtggcaggaagacaaggagcagacacaagatctccctggtccacatgccaccacctccctctgcagaggacaaggggatcctcatgctggcattggagggggttgagcagggcccaccttgagccctcaggagcacgaccacagcagccctgcagggagggattggtgggaggagagtcccaagtatcagggagaggagagttggtgtcccacaggagacctcagagccacaaggcgagcttgttcataaatttgggacccttagcatttcacagttatttgcagagcccagaaatggatgttactgaagctcacagttgcaagcatctgttaaatttttattagattttacttttagggaaaactttgaaatgctataaagaagcctgtgtttaaaagttaagacagaggctgggggcgatggctcacgcctgtaatctcagcactttgggaggccaaggcaggtggatcatttgaggttaggagttcgagaccagcctggccaacatggtgagaccctgtctctactaaaattacaaaaaattagctgggcgtggtggcgggcacctgtagtcccagctactggggaggctgaagcaggataagtgcttgaacccaggaggcagaggttacagtgagccaagatcacaccactgtaccctaagcctgggcgacagagtgagactctgtctcaaaaaataaaataaaataaagttaagagagaaaaaaatatatcctatatcctttgttaaattccaaaacagtaggggacaaataactgacttgacaggttactacaatatttcctgaaatgatgttttcttgaatactggcctactagaggttcataggtgtgtttggattaaaaaagagttccatggcccagtgactgggggaaaaaaataaaagactaaagtaagttaaacaggcttttctgctgcaggacttgtcagagcctttaatgtactaatggccattgtgaccctctgagaaggtcacagagtgggtttcccaaacttacttgattctacctgctaacatttcctggaggaagtttgggaaatgccgatttagcagattcttttgttgtgccgtggatggtgctggttgatgtgggcaaaacaaagaacacgtgagtcagatccgcctggggctcttactaaagtgcaggttcccaggtgccactttaggcttacagacccagttgtggggtaagcctgggagtcttttagcaggtgattctgccacatagtatagttggaaaacctctgggcatactcattgctggtccctctagaaatccaggtgacaatagccaatgagaagctccaagagacccagttgtccatggggtagagggaatgtgatattgaaaccaaagaagaaaatctatgatcagttttcagcagtgactgtcaagagaaggagaagggtgagttagcgctgatgctggctgacaggtcagcgggttggtttcaccaaggagtgtgatgaaggctgatgttgtctgtgggaatgtatgatggtaactggtttgtagctaatttggggaagcagtgagaattcgtgccctttgaagaccagtaagtggcaagaaacccaccaggcctggctcagggctgggctgggcttggctcgtctcagagcagctggggctggtggccaaagccaccattagtgaggggcaggccctgggggtacaaccagcaactaggggacaaagacaaccctgccagcctctcctattctggaggcgtgtgaccagaaatggagatgggttggtcagcataagatggccaggaaggtggaaatcaggactgctggcaatctagccacatgggcaggggagccgggtggttccaggcagtttccaaggccaagagggtgagcaggcacctcacagggaatcagggccaagcctggctgcagtgtggagacaatgcacccacccccatccttggatcttgcaggaggctgggtcctcactgagctaccaacatccatggccctgaggcttttaaaacacccatccatggagtggggctggtcccagtggggtgaggctgaccctggcagaaacagggcaggagcctgtgggttagggagactgcaccttccttagatagcctccatgccatcatgtccccgtgacagtttctgctgcgtcccctctgcatggtcccaccctcggccagcctgctgccccctcttgccaggttgcgctaatcagtgaccccagtgtgctgtgttgatactaacaatgcgaggcctagcagattcaagggaaaagagaaccaactgggtttccaccagacccaactaaacaaacatggacctatcccagagaaatccagcttcaccacagctggctttctgtgaacagtgaaaatggagtgtgacaagcattcttattttatattttatcagctcgcatggtcagtaaaagcaaagacgggactggaagcgatgacaaaaaagccaaggtaagctgacgatgccacggagctctgcagctggtcaagtttacagagaagctgtgctttatgtctgattcattctcatatataatgtggggagtatttgtcactaaagtacagctgtcatttaaagtgctttgtattttggggcaggcttttaaaaagtccagcatttattagttttgatacttaccccagggaagagcagttggcaggttcatgaagtcatgctcctaattccagctttcttagtgtactttcagtgagaccctgacagtaaatgaaggtgtgtttgaaaaccaaacccaggacagtaaatgaaggtgtgtttgaaaaccagccctaggacagtaaatgaagccatcttctcactgcataaactgcacccagatcttcgcccatccttctcagtatttcacttcacccattgtttactgtctcaatgactggggaaatgtctggggaaatgctcccgtaattgcacagtggcgtttttcctggaaaatcccaccatggctctagataagacctatttttcttaaaggtatctaaaatttccagcataaattctgtctgaaacacctgaattttaatcagtactggagcccggagggcatctccagttgccacatagctctgagcattcagtggtgtgttgagggctgctcccggaagtgcctgcagagtcagggctccccagcctcatctagtgaggcagtggaagggcctgtggggatttggagagctggcctgggtctctgaagtgatagtgacagctgcttgtcaatcacggtgcacatttagtgctgggggcagggggcagggaataccagcctcatgcatgcatgcattcatttgttccttccttcattcattcattcagtacacatgggtacaacatccctgccctggagttgcccagagtctagggaggggaaagatctattaccctgggcctcggccagctggggagtgctgctggtggagaggggccgtgtgcagcgagggaaggaggagtcgtcaatacccccaccccagctttgctttcttgtcatcagccccagggccccagcctgtgtccctcctctcccattgctacttcatctcctgggtcctccttaccaagcctgaccacacagagggccttggccgcttccatggggaattggaaagcaataagatagcatcccctagaagcccagtgaagtctgggacaggacccttctctgagctctgacttgctcttggaaacacttcgaggcttagcctccccactttgtttcccgagagtgtgacctgttcccctccaaacacccccttctcctccagggccatgcccacccgtcaaaatcccccacgggcaggacgaactgtgggtgtcagtcaccatctatcctgcatcctggttccagggccccccccagccccgcctccatagggacaggcgtgcagacacccgtccctggctgcttcctcttgtggaatgggttcaaaagtaagcagtgttgtttacactgacaaactgaaaaaaaaagaaaaagagataacattggaggcttggcacagtggctcatgcctgtaatcccagcactttgggaggctaaggtgggaggatgtccccagcccaagagttctagaccagcctgggcaacatagcaagaccccatctcaaaaaaaaaatttaattggccaggcagaggtgggaggatcacttgaacccaaagggtggaggctgcagtgagccgtgatggcaccactgcactccagccagggcaacagagggagaccctgtctctaaaacaaacaaacaaacaaacaaacaaaagagttaacattggccagattaggattcaccagatagtgttaatattagtttgatttgagactttaatcagaaagcacatgtgtggtgggggtgggtgtaacctaagtcaggtagaatctttccaacttggggggggcacactcctgattgtagccatatgagtctgtcagtgtggtggaagaggccatgggttaatgggcaggtaaaaaagcaccttgcctggaattgagtagaaagtaaggcccttcagaccccgtgacacacttggggacattttcttgagtaacatcctaagattcatgtaccttgatgatctccatcaacttactcatgtgaagcacctttaaaccagtcgtctccaaattcaggggcacagtaacatccaacaggctggagaaagaacgtactagaacttccattcctttttcatgtcctcttctaaaagctttgtcagggccaggcgcggtggctcacgcctgtaatcccagcactttgggaggccgagacgggtggatcacgaggtcaggagatcgagaccatcctggctaacacagtgaaaccccatctctactaaaaatacaaaaaaacgagccgggcgtggtggtgggcgcctgtagtcccagctactcgggaggctgaggcaggagaatggcgtgaacccaggaggcagagcttgcagtgagccgagattgcaccactgcagtccagcctgggcgacagagcgagactccgtctcaaaaaagaaaaagaaaaagaaaaagaactgtgattggggaggacggtcactttcctgttcttactgatcagaagggatattaagggtacctgattcaaacagcctggagatcactgctttcaaccattacctgccttatttatttttagttactgtccttttttcagtttgtttccctcctccatgtgctgacttttattttgattttatttatgtttatgtttaagacatccacacgttcctctgctaaaaccttgaaaaataggccttgccttagccccaaacaccccactcctggtagctcagaccctctgatccaaccctccagccctgctgtgtgcccagagccaccttcctctcctaaatacgtctcttctgtcacttcccgaactggcagttctggagcaaaggagatgaaactcaaggtaaggaaaccacctttgaaaagaaccaggctgctctgctgtggtttgcaaatgtggggtttgtttatttgttttttagcctcaaagacctttcttcaaatgagttctggcatagaagcaccgtgtaaaatagttagaattctgggcaaaggggaaaagagagctgggggccatccctctcagcaccccacaggctctcatagcagcagctcctaagacacctggtgggaccttggtttcgaaatcgctactctaaggctgggcacggtggctcacacctgtaatcccagctctttaggaggccgaggagggtggatcacctgagatcaggagttcgagaccagcctggctaacatggcaaaaccctgtctctactaaaaatacaaaaattagccgggcgtggtggtatgcgtggtggtaatcgcagctactcgggaggctgaggcacaaggattgcttgaaccccagaggcagaggttgtagttagctccagcttgggcgacagagcaagaccctgtcgcaaaaattgtttaaaaaacaaacccaaaattgctactctcattgggttcctttgcccattcctgattttggcaagagaaatgcttccagattgccctgatctgggtaggacagcatcacgccatagcaacactgccccgtgagctcactgccccctcaactagcttgtggtccttggttaatgtcagtttcttttttgagtttgtgttatgtctaagggtcatctgctgggtaacggaacccagggactgccctagtccctagactgtgccatgcccgactctgccagctttgtcagtgatgctggtgctcgcctcctcgggtgctcgcctggtctgagcacacccaaggagttcttgaggccttagggttgtttgcgagagaatgaaagaacacgacctagctctctttagcatccttggtcaggttcaacactgcccccaggggcctctggtggagccaaccaccatcagccaaataaatccataattagagtcagaaaatggatgtctgcatatgtgtagtgcactaatgtcctgccgatgattgacatggagtggagagtgacctgatcattgctgtgagctctgctggccttggcacaactcatgctgataactaatgcacacagttcctctgggaggaaatgtcctcagggaacttggagtttgggtggggatgtgggtttgtgtgcccagcaagcccttgtggttgtagcagacactagtggcatctaggaggcaaagggtcaccccagtcttagccacgttttgagtcaaggtggcggagtggggctggtgttgactcttggtggcagtaacttttcccaatggtgaaaaacccctctatcatgtttcatttacagggggctgatggtaaaacgaagatcgccacaccgcggggagcagcccctccaggccagaagggccaggccaacgccaccaggattccagcaaaaaccccgcccgctccaaagacaccacccagctctggtaagaagaacgttctcttgaatcttagaggaagctgaagctctcagaggtacagccttcattttaggaggccttaggccactgagaatgaataacccctggcagctggtcagcagcttgcagtttactaagcactggagtcttcattgccttctcagtccttttgatttctgaggcaaatgttgaatccctacctttttttttttttttcttttgagacagagtttcgcttttgttatccaggccggagtgcagtggtgtgatctcagctcactgcatcctccacctcccaggttcaagcgattctcctacctcagcctccctagtagctgggattacaggcacctgccactatgcccggctaattttttgtatttttagtagagacagggtttcaccatgttggccaggctggtctcgaacgcctgacctcaggtgatccacctgcctcggcctcccaaagtgctgggattacaggcatgagccaccactcccagcctgaatcctcactttttatcaatgaagaaattgaggctgattctgcagcatgataaaaaaaaatacagaaaaaggaaaaaaaagaaagaaatcgagcctctgagagtttgcttgactgagtctaaccagctcattttaaacccgaggaaaatgcagtcacatgactactaagtggcagctctcggagcctctctggccccaagtccagggttccatagaggcagccccagcatggcatgttttcagtccccaaatgagactctggagacaaatgtctctggagacagagcagcagcctggataagtcacaatgggtgacgtcactcagggctcaacccctgggcagcttaacttgctagggacgttaggagtctgctgcaaaacctgagggtcttagctgagcagtcacaggctgggcccgttgccctgggctcctgtgagtaaaacccagtcaattttgagtacccagtaaggcatccattgagttattttgcagccaggagtgctattaagaacagtcgcggctgggcgtggtggctcatgcctgtaatcccagcactttgggaggccaaggtgggcggatcacctgaggtcaggagttcgagaccagcttggccaacatggcaaaaccccgtctctaataaaaatacaaaataattagctgggcgtggtggcgggcgcctgtaatcccagcttctcaggagggtgaggaaggagaatcacttgaacccaggaggcagaggttgcagtgagctgagatcgcaccattgcactccagcctggatgacaaaagtgagattccttctcaaaaaaaaaaaaaaaaaaacagtcgtcctctttggggattagggacagcctgcctgcctgcccgagcacttctctcttccattgccccagtgaagtattccaggcccctgggtttagactctgcaccatgtaggggtgtctgacctgcacttgctccttggtggcacgggcagcctatggcacttgctgcgggctgtgaccaaagcctggcctggatcttggatcttggtgactctgcttctccctggcctgagggagctgcccagagcctgcccaccacctgctgcgtgtctttgcggtggcatttctcgcacacatgccgtgcagtggcacccccaaggatggccattcactaaggcccattgtttttgtcttttcgcttcgtgttttctggcctggtgtttttctcatatacatgtgatccagggataattcccagaattttgacaggattttaagtagcgtttggatcctgctgtttttttttcacttaacatcgggccagttgactcacactctgttttttgttgttgtttttttgagacggagtctcactgtgtcacccaggctgaagtgcagtggcacaatcttggcatactgcaacctctgcttcccaaattcaagcagttttcctgcctcagcctcctgagtagctgggactacaggcacaggccaccacgccctgctaatttttgtatttttagtaaagacagggtttcaccattttggccagcctagtctcgaactcctgacctcaagtgatccgcccacctcggcctcccaaagtgctgggattacaggggactcacactttgtaacaacctgaaacaacgtgatgcatttccctttgggtcttacctgctcttcggtggctgcctgcaggtggagagaccctcccccttgggcccctcgaccttgtttcagaatggggcccctgctgggccagctgtgggtgcctgccacgtgaaggactcattaaggccctgtttaagcctgatgataataaggctttcgtggatttttctctttaagcgactaagcaagtccagagaagaccaccccctgcagggcccagatctgagagaggtactcgggagcctacttcgctgggagcagcctccctttgcgtgtgtggccattcactggcttgtgtttctagagccgggaggacccttttctgcaatgcagggttcacacagggttcgcagcctgaagatggagcagtccgaattctcttccctgtgcagtttgcgcagctgtgtttgtctgatgggctttctaatcctgtgtgctctccttgacttcagggacaatggcattacaggcatgagccaccatgcctggctgtctccctatgtttcagatgaagacataggcttaaggaggtcaggtgacttgcccacgaccactctgtaaataagaggcatgaaaagtatttggagccaccaccaccaagcccactggtcaccctgggtctctgaagtcagggaggcaggaggatgggaggtctgaggaggcagagaggctgagcctggaggccctggaggccgaggccccatctgttgtttccttatgtggaaaataagaggcttcgtttgtcctattgccacagagcgtactacttcaggaacatccaagacatggaaatccgcagggcacggtggctcacgtctataatcccggcactttgggaggttgaggtgggagaatcgcttgaggccagaagttcaagaccagcctgagcaacatagtcagaccccgtctctataaaaaacattatttttaaaaaagacatggaagtcaaattctaaaaactggtgctggctgggtgcggtggctcatgcctataatcccagcactttgggaggccgaggcgggtggatcacctgaggtcaggagttcaagaccagcctggccaacatggtaaaacctctactaaagaaatctttactgaaaatacaaaaatccagtctctactaaaataagtctctactaaaaatacaaaaattagccaggcgtggtgctgcacacctgtaatatcagctactcgggaggctgaggcaggagactcgcttgatcccatgcagcggaggttgcagtgagccgagatcacgccattgcactccagcctgggcatcagaataagactccgtctcaaaaaaaaaaccacaaaaaaacaaaacaacaacaaaagaaaactagtgcttattcgtcactggccaagctgcccattggctacatgggtgcttcaaagagctgcccttctccaggtctggccagcaggtatgtgttacagcaaatgcctggggcagcggcaggggcattgctgcgggaagcttctggacttgcaggaaagctaagttctcagactgcaggggagctaagcacacctcggcacagggtgaggcctgcggttctcagacttcagtctttgtggagcttgagaaaaatgaggctttgcaggtcccacccctagagattctgctctatccactcttgaaggggatcgagaaatttgcattttgcaactcccactttcctccttgaaagctccggagattctgacgcagggttccgtgggccacactttggaaaatacagacccatgagatagaataccagactgttgaagtgtaacgggggcctgggaagtgcagtaacagaagcaagtttgagggtaaaggacacccagaggagggagggacagcatctgcatggagaggagaagagaccccccagcagcttccagggtgttggaagggtgcgctagtaactgctatgcatggcaggtggggaactgtacgtcagggcacagcagcatgaagcggtatggctcgtgtggacagctagggacaggcaggcgtggagcaggcatcctgttctgaaggccaaatcccacagaggagccagggtgctggcaggagccctgaactagccgaacagctgaacagctgaacattcaccctgtggggaaagggtcagaagcgtccaggcttgagggcacagctgggtctcgtcactgcatcacccttatttaggataaaggccctgaagaattgtattagaggttggcaaagcatatctaccacctcctggagccacgctggccgcagggattataattatttccattttcaaattaaggcctctgagctcagagaggggaagttacttgtctgaggccacacagcttgttggagcccatctcttgacccaaagactgtggagccgagttggccacctctctgggagcgggtattggatggtggttgatggttttccattgctttcctgggaaaggggtgtctctgtccctaagcaaaaaggcagggaggaagagatgcttccccagggcagccgtctgctgtagctgcgcttccaacctggcttccacctgcctaacccagtggtgagcctgggaatggacccacgggacaggcagcccccagggccttttctgaccccacccactcgagtcctggcttcactcccttccttccttcccaggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgagagtggctggctgcgcgtggaggtgtggggggctgcgcctggaggggtagggctgtgcctggaagggtagggctgcgcctggaggtgcgcggttgagcgtggagtcgtgggactgtgcatggaggtgtggggctccccgcacctgagcacccccgcataacaccccagtcccctctggaccctcttcaaggaagttcagttctttattgggctctccactacactgtgagtgccctcctcaggcgagagaacgttctggctcttctcttgccccttcagcccctgttaatcggacagagatggcagggctgtgtctccacggccggaggctctcatagtcagggcacccacagcggttccccacctgccttctgggcagaatacactgccacccataggtcagcatctccactcgtgggccatctgcttaggttgggttcctctggattctggggagattgggggttctgttttgatcagctgattcttctgggagcaagtgggtgctcgcgagctctccagcttcctaaaggtggagaagcacagacttcgggggcctggcctggatccctttccccattcctgtccctgtgcccctcgtctgggtgcgttagggctgacatacaaagcaccacagtgaaagaacagcagtatgcctcctcactagccaggtgtgggcgggtgggtttcttccaaggcctctctgtggccgtgggtagccacctctgtcctgcaccgctgcagtcttccctctgtgtgtgctcctggtagctctgcgcatgctcatcttcttataagaacaccatggcagctgggcgtagtggctcacgcctataatcccagcactttgggaggctgaggcaggcagatcacgaggtcaggagttcgagaccaacctgaccaacagggtgaaacctcgtctctactaaaaatacaaaaatacctgggcgtggtggtggtgcgcgcctataatcccagctactcaggaggctgaggcaggagaatcgcttgaacccaggaggcagaggttgcagtgagccgagatagtgccactgcactccagtttgagcaacagagcgagactctgtctcaaaacaaaataaaacaaaccaaaaaaacccaccatggcttagggcccagcctgatgacctcatttttcacttagtcacctctctaaaggccctgtctccaaatagagtcacattctaaggtacgggggtgttggggaggggggttagggcttcaacatgtgaatttgcggggaccacaattcagcccaggaccccgctcccgccacccagcactggggagctggggaagggtgaagaggaggctgggggtgagaaggaccacagctcactctgaggctgcagatgtgctgggccttctgggcactgggcctcggggagctagggggctttctggaaccctgggcctgcgtgtcagcttgcctcccccacgcaggcgctctccacaccattgaagttcttatcacttgggtctgagcctggggcatttggacggagggtggccaccagtgcacatgggcaccttgcctcaaaccctgccacctccccccacccaggatcccccctgcccccgaacaagcttgtgagtgcagtgtcacatcccatcgggatggaaatggacggtcgggttaaaagggacgcatgtgtagaccctgcctctgtgcatcaggcctcttttgagagtccctgcgtgccaggcggtgcacagaggtggagaagactcggctgtgccccagagcacctcctctcatcgaggaaaggacagacagtggctcccctgtggctgtggggacaagggcagagctccctggaacacaggagggagggaaggaagagaacatctcagaatctccctcctgatggcaaacgatccgggttaaattaaggtccggccttttcctgctcaggcatgtggagcttgtagtggaagaggctctctggaccctcatccaccacagtggcctggttagagaccttggggaaataactcacaggtgacccagggcctctgtcctgtaccgcagctgagggaaactgtcctgcgcttccactggggacaatgcgctccctcgtctccagactttccagtcctcattcggttctcgaaagtcgcctccagaagccccatcttgggaccaccgtgactttcattctccagggtgcctggccttggtgctgcccaagaccccagaggggccctcactggcctttcctgccttttctcccattgcccacccatgcacccccatcctgctccagcacccagactgccatccaggatctcctcaagtcacataacaagcagcacccacaaggtgctcccttccccctagcctgaatctgctgctccccgtctggggttccccgcccatgcacctctgggggcccctgggttctgccataccctgccctgtgtcccatggtggggaatgtccttctctccttatctcttcccttcccttaaatccaagttcagttgccatctcctccaggaagtcttcctggattcccctctctcttcttaaagcccctgtaaactctgaccacactgagcatgtgtctgctgctccctagtctgggccatgagtgagggtggaggccaagtctcatgcatttttgcagcccccacaagactgtgcaggtggccggccctcattgaatgcggggttaatttaactcagcctctgtgtgagtggatgattcaggttgccagagacagaaccctcagcttagcatgggaagtagcttccctgttgaccctgagttcatctgaggttggcttggaaggtgtgggcaccatttggcccagttcttacagctctgaagagagcagcaggaatggggctgagcagggaagacaactttccattgaaggcccctttcagggccagaactgtccctcccaccctgcagctgccctgcctctgcccatgaggggtgagagtcaggcgacctcatgccaagtgtagaaaggggcagatgggagccccaggttatgacgtcaccatgctgggtggaggcagcacgtccaaatctactaaagggttaaaggagaaagggtgacttgacttttcttgagatattttgggggacgaagtgtggaaaagtggcagaggacacagtcacagcctcccttaaatgccaggaaagcctagaaaaattgtctgaaactaaacctcagccataacaaagaccaacacatgaatctccaggaaaaaagaaaaagaaaaatgtcatacagggtccatgcacaagagcctttaaaatgacccgctgaagggtgtcaggcctcctcctcctggactggcctgaaggctccacgagcttttgctgagacctttgggtccctgtggcctcatgtagtacccagtatgcagtaagtgctcaataaatgtttggctacaaaagaggcaaagctggcggagtctgaagaatccctcaaccgtgccggaacagatgctaacaccaaagggaaaagagcaggagccaagtcacgtttgggaacctgcagaggctgaaaactgccgcagattgctgcaaatcattgggggaaaaacggaaaacgtctgttttcccctttgtgcttttctctgttttcttctttgtgcttttctctgttttcaggatttgctacagtgaacatagattgctttggggccccaaatggaattattttgaaaggaaaatgcagataatcaggtggccgcactggagcaccagctgggtaggggtagagattgcaggcaaggaggaggagctgggtggggtgccaggcaggaagagcccgtaggccccgccgatcttgtgggagtcgtgggtggcagtgttccctccagactgtaaaagggagcacctggcgggaagagggaattcttttaaacatcattccagtgcccgagcctcctggacctgttgtcatcttgaggtgggcctcccctgggtgactctagtgtgcagcctggctgagactcagtggccctgggttcttactgctgacacctaccctcaacctcaaccactgcggcctcctgtgcaccctgatccagtggctcattttccactttcagtcccagctctatccctatttgcagtttccaagtgcctggtcctcagtcagctcagacccagccaggccagcccctggttcccacatcccctttgccaagctcatccccgccctgtttggcctgcgggagtgggagtgtgtccagacacagagacaaaggaccagcttttaaaacattttgttggggccaggtgtggtggctcacacctaatcccaacacctggggaggccaaggcagaaggatcacttgagtccaggagttcaagaccagcctgggcaacatagggagaccctgtctctacaattttttttttaattagctgggcctgttggcactctcctgtagttccagctactctagaggctgaggtgggaggactgcttgagcctgggaggtcagggctgcaatgagccatgttcacaccactgaacgccagcctgggcgagaccctgtatcaaaaaagtaaagtaaaatgaatcctgtacgttatattaaggtgccccaaattgtacttagaaggatttcatagttttaaatacttttgttatttaaaaaattaaatgactgcagcatataaattaggttcttaatggaggggaaaaagagtacaagaaaagaaataagaatctagaaacaaagataagagcagaaataaaccagaaaacacaaccttgcactcctaacttaaaaaaaaaaatgaagaaaacacaaccagtaaaacaacatataacagcattaagagctggctcctggctgggcgcggtggcgcatgcctgtaatcccaacactttgggaggccgatgctggaggatcacttgagaccaggagttcaaggttgcagtgagctatgatcataccactacaccctagcctgggcaacacagtgagactgagactctattaaaaaaaaaatgctggttccttccttatttcattcctttattcattcattcagacaacatttatggggcacttctgagcaccaggctctgtgctaagagcttttgcccccagggtccaggccaggggacaggggcaggtgagcagagaaacagggccagtcacagcagcaggaggaatgtaggatggagagcttggccaggcaaggacatgcagggggagcagcctgcacaagtcagcaagccagagaagacaggcagacccttgtttgggacctgttcagtggcctttgaaaggacagcccccacccggagtgctgggtgcaggagctgaaggaggatagtggaacactgcaacgtggagctcttcagagcaaaagcaaaataaacaactggaggcagctggggcagcagagggtgtgtgttcagcactaaggggtgtgaagcttgagcgctaggagagttcacactggcagaagagaggttggggcagctgcaagcctctggacatcgcccgacaggacagagggtggtggacggtggccctgaagagaggctcagttcagctggcagtggccgtgggagtgctgaagcaggcaggctgtcggcatctgctggggacggttaagcaggggtgagggcccagcctcagcagcccttcttggggggtcgctgggaaacatagaggagaactgaagaagcagggagtcccagggtccatgcagggcgagagagaagttgctcatgtggggcccaggctgcaggatcaggagaactggggaccctgtgactgccagcggggagaagggggtgtgcaggatcatgcccagggaagggcccaggggcccaagcatgggggggcctggttggctctgagaagatggagctaaagtcactttctcggaggatgtccaggccaatagttgggatgtgaagacgtgaagcagcacagagcctggaagcccaggatggacagaaacctacctgagcagtggggctttgaaagccttggggcggggggtgcaatattcaagatggccacaagatggcaatagaatgctgtaactttcttggttctgggccgcagcctgggtggctgcttccttccctgtgtgtattgatttgtttctcttttttgagacagagtcttgctgggttgcccaggctggagtgcagtggtgcgatcatagctcactgcagccttgaagtcctgagctcaagagatccttccacctcagcctcctgagtagttgggaccacaggcttgcaccacagtgcccaactaatttcttatattttttgtagagatggggtttcactgtgtcgcccaggatggtcttgaactcctgggctcaagtgatcctcctgcctcagcctcgcaaattgctgggattacaggtgtgagccaccatgcccgaccttctctttttaagggcgtgtgtgtgtgtgtgtgtgtgtgggcgcactctcgtcttcaccttcccccagccttgctctgtctctacccagtcacctctgcccatctctccgatctgtttctctctccttttacccctctttcctccctcctcatacaccactgaccattatagagaactgagtattctaaaaatacattttatttatttattttgagacagagtctcactctgtcacccaggctggagtgcagtggtgcaatctcggctcactgcaacctccgcctcccaggttgaagcaactctcctgcctcagcctccctagtagctgggattacaagcacacaccaccatgcctagcaaatttttatatttttagtagaggaggggtgtcaccatgtttgccaagctggtctcaaactcctggcctcaggtgatctgcctaccttggtctcccaaagtgctgggattacaggtgtgagccaccacgcctgcccttaaaaatacattatatttaatagcaaagccccagttgtcactttaaaaagcatctatgtagaacatttatgtggaataaatacagtgaatttgtacgtggaatcgtttgcctctcctcaatcagggccagggatgcaggtgagcttgggctgagatgtcagaccccacagtaagtggggggcagagccaggctgggaccctcctctaggacagctctgtaactctgagaccctccaggcatcttttcctgtacctcagtgcttctgaaaaatctgtgtgaatcaaatcattttaaaggagcttgggttcatcactgtttaaaggacagtgtaaataattctgaaggtgactctaccctgttatttgatctcttctttggccagctgacttaacaggacatagacaggttttcctgtgtcagttcctaagctgatcaccttggacttgaagaggaggcttgtgtgggcatccagtgcccaccccgggttaaactcccagcagagtattgcactgggcttgctgagcctggtgaggcaaagcacagcacagcgagcaccaggcagtgctggagacaggccaagtctgggccagcctgggagccaactgtgaggcacggacggggctgtggggctgtggggctgcaggcttggggccagggagggagggctgggctctttggaacagccttgagagaactgaacccaaacaaaaccagatcaaggtctagtgagagcttagggctgctttgggtgctccaggaaattgattaaaccaagtggacacacacccccagccccacctcaccacagcctctccttcagggtcaaactctgaccacagacatttctcccctgactaggagttccctggatcaaaattgggagcttgcaacacatcgttctctcccttgatggtttttgtcagtgtctatccagagctgaagtgtaatatatatgttactgtagctgagaaattaaatttcaggattctgatttcataatgacaaccattcctcttttctctcccttctgtaaatctaagattctataaacggtgttgacttaatgtgacaattggcagtagttcaggtctgctttgtaaatacccttgtgtctattgtaaaatctcacaaaggcttgttgccttttttgtggggttagaacaagaaaaagccacatggaaaaaaaatttcttttttgtttttttgtttgcttgtttttttgagacagagtttcactctgtcgcccaggctggagtgcagtggtgcgatctccgcccactgcaagctccacctcccgggttcatgctattctcctgtctcagcctcccaagtagctgggactgcaggtgcccgccaccacacctggctaatttttttgtatttttagtagagacggggtttcaccgtgttagccaggatggtctcaatctcctgacctcgtcatctgcctgcctcggcctcccaaagtgctgagattacaggcgtgagccaccgtgcccggccagaaaaaaacatttctaagtatgtggcagatactgaattattgcttaatgtcctttgattcatttgtttaatttctttaatggattagtacagaaaacaaagttctcttccttgaaaaactggtaagttttctttgtcagataaggagagttaaataacccatgacatttccctttttgcctcggcttccaggaagctcaaagttaaatgtaatgatcactcttgtaattatcagtgttgatgcccttcccttcttctaatgttactctttacattttcctgctttattattgtgtgtgttttctaattctaagctgttcccactcctttctgaaagcaggcaaatcttctaagccttatccactgaaaagttatgaataaaaaatgatcgtcaagcctacaggtgctgaggctactccagaggctgaggccagaggaccacttgagcccaggaatttgagacctgggctgggcagcatagcaagactctatctccattaaaactatttttttttatttaaaaaataatccgcaaagaaggagtttatgtgggattccttaaaatcggagggtggcatgaattgattcaaagacttgtgcagagggcgacagtgactccttgagaagcagtgtgagaaagcctgtcccacctccttccgcagctccagcctgggctgaggcactgtcacagtgtctccttgctggcaggagagaatttcaacattcaccaaaaagtagtattgtttttattaggtttatgaggctgtagccttgaggacagcccaggacaactttgttgtcacatagatagcctgtggctacaaactctgagatctagattcttctgtggctgcttctgacctgagaaagttgcggaacctcagcgagcctcacatggcctccttgtccttaacgtggggacggtgggcaagaaaggtgatgtggcactagagatttatccatctctaaaggaggagtggattgtacattgaaacaccagagaaggaattacaaaggaagaatttgagtatctaaaaatgtaggtcaggcgctcctgtgttgattgcagggctattcacaatagccaagatttggaagcaacccaagtgtccatcaacagacaaatggataaagaaaatgtggtgcatatacacaatggaatactattcagccatgaaaaagaatgagaatctgtcatttgaaacaacatggatggaactggaggacattatgttaagtgaaataagccagacagaaggacagacttcacatgttctcacacatttgtgggagctaaaaattaaactcatggagatagagagtagaaggatggttaccagaggctgaggagggtggaggggagcagggagaaagtagggatggttaatgggtacaaaaacgtagttagcatgcatagatctagtattggatagcacagcagggtgacgacagccaacagtaatttatagtacatttaaaaacaactaaaagagtgtaattggactggctaacatggtgaaaccccgtctctactaaaaatacaaaaattagctgggcatggtggctcacgcctgtaatcccagcactttgggaggccgaggcgggccgatcacgaggtcaggagatcgagaccatcctagctaacatggtgaaaccccgtctctactacaaatacaaaaaaaagaaaaaattagccgggcatggtggtgggcgcctgtagtcccagctactcgggaggctgaggcaggagaatggcgtgaacccgggaggcggagcttgcagtgagccgagatcgcgccactgcactccagcctgggcgacaaggcaagattctatctcaaaaaaataaaaataaaataaaataaaataataaaataaaataaaataaaataaaataaataaaataaaataaaatgtataattggaatgtttataacacaagaaatgataaatgcttgaggtgatagataccccattcaccgtgatgtgattattgcacaatgtatgtctgtatctaaatatctcatgtaccccacaagtatatacacctactatgtacccatataaatttaaaattaaaaaattataaaacaaaaataaataagtaaattaaaatgtaggctggacaccgtggttcacgcctgtaatcccagtgctttgtgaggctgaggtgagagaatcacttgagcccaggagtttgagaccggcctgggtgacatagcgagaccccatcatcacaaagaatttttaaaaattagctgggcgtggtagcacataccggtagttccagctacttgggagaccgaggcaggaggattgcttgagcccaggagtttaaggctgcagtgagctacgatggcgccactgcattccagcctgggtgacagagtgagagcttgtctctattttaaaaataataaaaagaataaataaaaataaattaaaatgtaaatatgtgcatgttagaaaaaatacacccatcagcaaaaagggggtaaaggagcgatttcagtcataattggagagatgcagaataagccagcaatgcagtttcttttattttggtcaaaaaaaataagcaaaacaatgttgtaaacacccagtgctggcagcaatgtggtgaggctggctctctcaccagggctcacagggaaaactcatgcaacccttttagaaagccatgtggagagttgtaccgagaggttttagaatatttataactttgacccagaaattctattctaggactctgtgttatgaaaataacccatcatatggaaaaagctcctttcagaaagaggttcatgggaggctgtttgtattttttttttctttgcatcaaatccagctcctgcaggactgtttgtattattgaagtacaaagtggaatcaatacaaatgttggatagcaggggaacaatattcacaaaatggaatgggacatagtattaaacatagtgcttctgatgaccgtagaccatagacaatgcttaggatatgatatcacttcttttgttgttttttgtattttgagacgaagtctcattctgtcacccaggctggagttcagtggcgccatctcagctcactgcaacctccatctcccgggttcaagctattctccttcctcaacctcccgagtagctgggttgcgcaccaccatgcctggctaacttttgtatttttagtacagacggggtttcaccacgttggccaggctgctcttgaactcctgacgtcaggtgatccaccagccttgacctcccaaagtgctaggattacaggagccactgtacccagcctaggatatgatatcacttcttagagcaagatacaaaattgcatgtgcacaataattctaccaagtataggtatacaggggtagttatatataaatgagacttcaaggaaatacaacaaaatgcaatcgtgattgtgttagggtggtaagaaaacggtttttgctttgatgagctctgttttttaaaatcgttatattttctaataaaaatacatagtcttttgaaggaacataaaagattatgaagaaatgagttagatattgattcctattgaagattcagacaagtaaaattaaggggaaaaaaaacgggatgaaccagaagtcaggctggagttccaaccccagatccgacagcccaggctgatggggcctccagggcagtggtttccacccagcattctcaaaagagccactgaggtctcagtgccattttcaagatttcggaagcggcctgggcacggctggtccttcactgggatcaccacttggcaattatttacacctgagacgaataaaaaccagagtgctgagattacaggcatggtggcttacgcttgtaatcggctttgggaagccgaggtgggctgattgcttgagcccaggagtttcaaactatcctggacaacatagcatgacctcgtctctacaaaaaatacaaaaaatttgccaggtgtggtggcatgtgcctgtggtcccagctacttgggaggctgaagtaggagaatcccctgagccctgggaagtcgaggctgcactgagccgtgatggtgtcactgcactccagcctgggtgacaaagtgagaccctatctcacaaagaaaaaaaacaaaacaaaaaacccaaagcacactgtttccactgtttccagagttcctgagaggaaaggtcaccgggtgaggaagacgttctcactgatctggcagagaaaatgtccagtttttccaactccctaaaccatggttttctatttcatagttcttaggcaaattggtaaaaatcatttctcatcaaaacgctgatattttcacacctccctggtgtctgcagaaagaaccttccagaaatgcagtcgtgggagacccatccaggccacccctgcttatggaagagctgagaaaaagccccacgggagcatttgctcagcttccgttacgcacctagtggcattgtgggtgggagagggctggtgggtggatggaaggagaaggcacagcccccccttgcagggacagagccctcgtacagaagggacaccccacatttgtcttccccacaaagcggcctgtgtcctgcctacggggtcagggcttctcaaacctggctgtgtgtcagaatcaccaggggaacttttcaaaactagagagactgaagccagactcctagattctaattctaggtcagggctaggggctgagattgtaaaaatccacaggtgattctgatgcccggcaggcttgagaacagccgcagggagttctctgggaatgtgccggtgggtctagccaggtgtgagtggagatgccggggaacttcctattactcactcgtcagtgtggccgaacacatttttcacttgacctcaggctggtgaacgctcccctctggggttcaggcctcacgatgccatccttttgtgaagtgaggacctgcaatcccagcttcgtaaagcccgctggaaatcactcacacttctgggatgccttcagagcagccctctatcccttcagctcccctgggatgtgactcaacctcccgtcactccccagactgcctctgccaagtccgaaagtggaggcatccttgcgagcaagtaggcgggtccagggtggcgcatgtcactcatcgaaagtggaggcgtccttgcgagcaagcaggcgggtccagggtggcgtgtcactcatccttttttctggctaccaaaggtgcagataattaataagaagctggatcttagcaacgtccagtccaagtgtggctcaaaggataatatcaaacacgtcccgggaggcggcagtgtgagtaccttcacacgtcccatgcgccgtgctgtggcttgaattattaggaagtggtgtgagtgcgtacacttgcgagacactgcatagaataaatccttcttgggctctcaggatctggctgcgacctctgggtgaatgtagcccggctccccacattcccccacacggtccactgttcccagaagccccttcctcatattctaggagggggtgtcccagcatttctgggtcccccagcctgcgcaggctgtgtggacagaatagggcagatgacggaccctctctccggaccctgcctgggaagctgagaatacccatcaaagtctccttccactcatgcccagccctgtccccaggagccccatagcccattggaagttgggctgaaggtggtggcacctgagactgggctgccgcctcctcccccgacacctgggcaggttgacgttgagtggctccactgtggacaggtgacccgtttgttctgatgagcggacaccaaggtcttactgtcctgctcagctgctgctcctacacgttcaaggcaggagccgattcctaagcctccagcttatgcttagcctgcgccaccctctggcagagactccagatgcaaagagccaaaccaaagtgcgacaggtccctctgcccagcgttgaggtgtggcagagaaatgctgcttttggcccttttagatttggctgcctcttgccaggagtggtggctcgtgcctgtaattccagcactttgggagactaaggcgggaggttcgcttgagcccaggagttcaagaccagcctgggcaacaatgagacccctgtgtctacaaaaagaattaaaattagccaggtgtggtggcacgcacctgtagtcccagctacttgggaggctgaggtgggaggattgcctgagtccgggaggcggaagttgcaaggagccatgatcgcgccactgcacttcaacctaggcaacagagtgagactttgtctcaaaaaacaatcatataataattttaaaataaatagatttggcttcctctaaatgtccccggggactccgtgcatcttctgtggagtgtctccgtgagattcgggactcagatcctcaagtgcaactgacccacccgataagctgaggcttcatcatcccctggccggtctatgtcgactgggcacccgaggctcctctcccaccagctctcttggtcagctgaaagcaaactgttaacaccctggggagctggacgtatgagacccttggggtgggaggcgttgatttttgagagcaatcacctggccctggctggcagtaccgggacactgctgtggctccggggtgggctgtctccagaaaatgcctggcctgaggcagccacccgcatccagcccagagggtttattcttgcaatgtgctgctgcttcctgccctgagcacctggatcccggcttctgccctgaggccccttgagtcccacaggtagcaagcgcttgccctgcggctgctgcatggggctaactaacgcttcctcaccagtgtctgctaagtgtctcctctgtctcccacgccctgctctcctgtccccccagtttgtctgctgtgaggggacagaagaggtgtgtgccgcccccacccctgcccgggcccttgttcctgggattgctgttttcagctgtttgagctttgatcctggttctctggcttcctcaaagtgagctcggccagaggaggaaggccatgtgctttctggttgaagtcaagtctggtgccctggtggaggctgtgctgctgaggcggagctggggagagagtgcacacgggctgcgtggccaacccctctgggtagctgatgcccaaagacgctgcagtgcccaggacatctgggacctccctggggcccgcccgtgtgtcccgcgctgtgttcatctgcgggctagcctgtgacccgcgctgtgctcgtctgcgggctagcctgtgtcccgcgctctgcttgtctgcggtctagcctgtgacctggcagagagccaccagatgtcccgggctgagcactgccctctgagcaccttcacaggaagcccttctcctggtgagaagagatgccagcccctggcatctgggggcactggatccctggcctgagccctagcctctccccagcctgggggccccttcccagcaggctggccctgctccttctctacctgggacccttctgcctcctggctggaccctggaagctctgcagggcctgctgtccccctccctgccctccaggtatcctgaccaccggccctggctcccactgccatccactcctctcctttctggccgttccctggtccctgtcccagcccccctccccctctcacgagttacctcacccaggccagagggaagagggaaggaggccctggtcataccagcacgtcctcccacctccctcggccctggtccaccccctcagtgctggcctcagagcacagctctctccaagccaggccgcgcgccatccatcctccctgtcccccaacgtccttgccacagatcatgtccgccctgacacacatgggtctcagccatctctgccccagttaactccccatccataaagagcacatgccagctgacaccaaaataattcgggatggttccagtttagacctaagtggaaggagaaaccaccacctgccctgcaccttgttttttggtgaccttgataaaccatcttcagccatgaagccagctgtctcccaggaagctccagggcggtgcttcctcgggagctgactgataggtgggaggtggctgcccccttgcaccctcaggtgaccccacacaaggccactgctggaggccctggggactccaggaatgtcaatcagtgacctgccccccaggccccacacagccatggctgcatagaggcctgcctccaagggacctgtctgtctgccactgtggagtccctacagcgtgccccccacaggggagctggttctttgactgagatcagctggcagctcagggtcatcattcccagagggagcggtgccctggaggccacaggcctcctcatgtgtgtctgcgtccgctcgagcttactgagacactaaatctgttggtttctgctgtgccacctacccaccctgttggtgttgctttgttcctattgctaaagacaggaatgtccaggacactgagtgtgcaggtgcctgctggttctcacgtccgagctgctgaactccgctgggtcctgcttactgatggtctttgctctagtgctttccagggtccgtggaagcttttcctggaataaagcccacgcatcgaccctcacagcgcctcccctctttgaggcccagcagataccccactcctgcctttccagcaagatttttcagatgctgtgcatactcatcatattgatcacttttttcttcatgcctgattgtgatctgtcaatttcatgtcaggaaagggagtgacatttttacacttaagcgtttgctgagcaaatgtctgggtcttgcacaatgacaatgggtccctgtttttcccagaggctcttttgttctgcagggattgaagacactccagtcccacagtccccagctcccctggggcagggttggcagaatttcgacaacacatttttccaccctgactaggatgtgctcctcatggcagctgggaaccactgtccaataagggcctgggcttacacagctgcttctcattgagttacacccttaataaaataatcccattttatcctttttgtctctctgtcttcctctctctctgcctttcctcttctctctcctcctctctcatctccaggtgcaaatagtctacaaaccagttgacctgagcaaggtgacctccaagtgtggctcattaggcaacatccatcataaaccaggtagccctgtggaaggtgagggttgggacgggagggtgcagggggtggaggagtcctggtgaggctggaactgctccagacttcagaaggggctggaaaggatattttaggtagacctacatcaaggaaagtgttgagtgtgaaacttgcgggagcccaggaggcgtggtggctccagctcgctcctgcccaggccatgctgcccaagacaaggtgaggcgggagtgaagtgaaataaggcaggcacagaaagaaagcacatattctcggccgggcgctgtggctcacgcctgtaattccagcactttgggaggccaaggtgggtggatcatgaggtcaggagattgagaccatcctggctaacacagtgaaaccccgtctctactaaaaatacaaaaaattagccgggcgtggtggtgggcgcctgtagtcccagctactccggaggctgaggcaggaaaatggcgtgaacccggaaggcggagcttgcagtgagcggagtgagcagagatcgcgccactgcactccagcctgggcgacagagcgagactccgtctcaaaaaaaaaaagcacatgttctcgcttctttgtgggatccaggagatagagaatagaaggatggttaccagaggctgggaagggtagtgaggggatggtggggggatggtcaatgggtacaaaaaaaatagaataagacctagtatttgatagtgcaacagggtgactatagtcaataataatttaattgtacatttaaaaataactaaaagatagccgggtgcagtggcttacgtctgtaatcccagtactttgggaggctgaggtgggcgtttgagaccagcctggccaacatggtgaaaccccatctctactaaaaatacaaaaattagccaggcatggtggcgggcgcctgtaatcccagctactcgggaggctgaggcaggagaatcacttgaacctgggaggcagaggttgcagtgagccgagatcttgccactgcactccagcctgggtgacagtgaaactccgtctcaaaaataaaaataaaaatacagctgggcacggtggctcacgcctgtaatcccagcactttgggaggccgaggcgagcggatcacaaggtcaggagatatagaccatcctggctaacacggtgaaacccggtctctactaaaaatacaaaaaattagccaggcgtggtggcaggtgcctatagtcccagctactcacaaggctgaggcaggagaatggcatgaacctgggaggcggagcttgcagtgagccgagattgtgccactgcactccagcctgggcgagagagtgagactccgtctcaaaacaaaaacaaaaacaaaaacaaaaacaaacacacaacaaaaacctaaaagaatataaatggattgtttgtaacacaaaggacaaatgtttgaggggatggataccccattttccatgatgtgattattatacattgtgtgtctgtatcaaaacatctcatgagccccataaatatatacacctaactatgtacccacaaaaattaaaaaaatatattttttaaggtgaagagggaggcgagatgctggccttaacccctaacccgttgttctccctgcaagctgtccacagggcctctcagactcgaggttcagctatatggatgcatgagcttggtccccagccaacatgggagacacttcaccatcggcagcagctacagcacaggaaccctgggtcactgccatgtcccctctgtgactttgtttaaacagaaaatgatgctctgggccggctgtggtggcccacacctataatcccagcaccttgggaggcgggggtgggcagattgcctgaggtcaggagttggagatcagcctggccgacatggcgaaaccccatgtctactaaaaatacaaaaactagccaggcatggtggcacatgcctgtaatcccagctacttgggaggctgaagcaggagaatcacttgaacccaggaggcagaggctgagtgagccaagatcgtgccaatgcactccagcttgggtgagggagtgagactccgtctcaaaaaaaaaaaaaaagaaagaaaaagaaaagaaagtgatcctactggaaccatgcttactcccctccccacctcacactgtgtagaaattagtgctgtcggccaggcgcggtggctcatgcctgtaatcgcagcactttgggaggccaaggcaggcggatcacgaggtcaggagatcaagaccatcctggctaacacagtgaaaccctgtctctactaaaaatacaaaaaattagccgggcatggtggcaggcacctgtagtcccaactacttgggaggctgaggcaggagaatggcatgaacctgggaggcggagcttgcagtgagccaagatcgcgccactgcataccagcctaggtgacagagtgagactcagcaaaaaaagaaagaaagaaagaaagaaatcagtgctgtctatacttctttctgcagtgatggaaatattctgtatctgtgctgtccagtatagtagccactagctacatgtggcacttgaaacatggctggtacagttgaggaagagtggctgccatatcggacgacacagctatagattctgtcaccccaccccgagagtccagagcggggacttctgccttaggccctattcagggctgatttttacttgaacccttactgtgggaagagaaggccatgagaagttcagtctagaatgtgactccttattttctggctcccttggacactttgtgggatttagtctccctgtggaaagtattccacaagtggtgccaccaccccagctgtgagagcagctgggagctgcttttgtcatctttccctggaaagtcctgtgggctgtctcttcctcatgccttgtcccatgcttgggcatggtgtcaagcgtcaggagggagaaagggtccttatttatttatttagagagggacccttcttctgttcccaggctggagtgcagtggtgcgatctcggctcactgcaacctccgcctcctgggttcaagtgattctcctgcctcagcctcctgagtagctgagattacaggcacatgccaacatgcctggctaattttttttttttttttttttttttttttttgagatggagttgtactctcattgcccaggctggaatgtaatggcacaatctcggctcactgcaacctccacctcctggattcaagcaattctcctgtctcagcttcccaagtagctgggattacaggtgcccgccaccatgctcaactaatttttgtattttttttttagtagagacgaggtttcaccatgttggtcagactggtctcaaactcctgacctcaggtgatccacctgcctcggcctcccaaagtgctaggattacaggcatgagccaccacgcccggcctgaaagggttcttatttagtgtgcattttgacattcaatttaattccaaggtcttgtggggtcatggtttacaggatgttgatatagaaaagacttcacttaatgggccgggcgcagtggctcatgcctgtaatcccagcactttgggaggccgaggcaggcagatcaggaggtcaggagattgagaccatcctggctaacacagtgaaaccccatctctactgaaaatacaaaaaattagctgggcgtggtggcaggcacctgtagtcccagccactcggttggctgaggcaggagaatggcatgaacccgggaggcggagcttgcagtgagcagagaccatgccactgcactccagcctgggcgacagagcaagactctgtctcaagaaaaaaaaaaaaaaaacagactttacttactggaagccaaccaatgtatatttagagtaatttttcctgggctgagctgtcatttacttttgcagtatctcaagaagaagagtttacagtgtaaatatttgatgcacactttgattatatagatgaagcaaactattttcaagagctttgcaaggacttacttgtatccaaacaccattctaaaggagtcttacctacttctaaaggctggtctctacttggaaccacttgcttggccctggttcaagtcctgctgcaaacctggaagtcctgtcattgtcttcttccctccagagcagtggcacccaatctaatttttgctgtgccccagcagcccctggcactttgccctgtagactgcagacctcatgtaatgtatgttaagtccacagaaccacagaagatgatggcaagatgctcttgtgtgtgttgtgttctaggaggtggccaggtggaagtaaaatctgagaagcttgacttcaaggacagagtccagtcgaagattgggtccctggacaatatcacccacgtccctggcggaggaaataaaaaggtaaagggggtagggtgggttggatgctgcccttgggtatatgggcattaatcaagttgagtggacaaaggctggtccagttcccagaggaggaaaacagaggcttctgtgttgactggctggatgtgggccctcagcagcatccagtgggtctccactgcctgtctcaatcacctggagctttagcacgtttcacacctgggccccaacctggagaggctgaccaatgggtctcaggggcagctcggttgctggagtttttgtttttatttatttttatgtatttaaggcagggtctctgtattagtccattctcacactgctaataaagacatacccaagactgggtaatttataaaggaaagaggtttaatggactcacagttccacatggctggggaggcctcaaaatcatggcggaaggcaaaggagaagcaaaggcatttcttacatggcgacaggcaagagagcgtgtgcaggggaactcccatttataaaaccatcagacctcatgagatttattcactatcatgagaacagcatgggaaagacccgcccccatgattcagttacctcccactgggtccctcccatgacacatggaattatgggagctacaattcaagatgagatttgggtggggacacagccaaaccatatcagtctccctctgtcatccaggctggagtgcactggcatgatctcggctcactgcagcctctacctccctgggtcaggtgatcttcccacctcagcctcccaggtagctggaactacaggtacctgccactatgcctggctaaatattttgtatttcctgtggagacgaggttttgccacgttgcccaggctggtcttgaactcctgaggtcaagcaatatgcccacctcggcctcccaaggtgctgggattacaggtgtgagccacagtgctcggcctaagtcactgcagtttttaaagctcccaggtgattcttcagtgcagtcaaaagtgagaactggctgggtgcggtggctcatgcctgtaatcccagcaccttgggaggcgaaggtgggcagatggcttgaggtcaggagttcaagaccagcctggccaacatggtaaaaccccatctctactaaaaatacaaaagttagctgggtgtggtggtgcgtgcctgtaatcccagctacttgggaggctgaggcatgagaattgcttgaacccaggggacagaggttgtagtgagccgagatcgtgccactgcactccagcctgggcaacagagtgagattccatctcacaaaaaaaaaaaaaaaagcgagaaccactgtcctaggccctgatgtttgcaggcaactaaaaaaggaagtggacatccccagtcagctgtggcgcaccaagaacaagtcatgggaacataacctaattttctaaatgggttactaggcacttagagcaaaacaatgatgccgaaatcctgatttcagcaaagcctctgcctgcctgtcttggaagtatccacatgaggctgctggggccttggtgtccccagcagtttctagtctctaggtcttgctgtgggtgtctgtgcagtgagggtgtgtgtggcgctgggtgagctctgtctaggcctggcacaggatgcggtctggtagctgctgcttctcttctgcagaagcgcagccaagcaccctctggggtttcaggcccacacccagcctgaagttctgggagtggctcactttccaaccttcagggtctcccagcagctgactggggagtggtggagggaaaagggattgtattagtccgttttcacgccgctgatgaagacatacccgatactgggcagtctaaaagatagaggtctgatggactcacagttccacgtgactggggaggcctgacaatcatggtggaaggtgaaaggcttgtctcacacggtggcagacaagagaaaagagcttgtgcaggggaactcccctttataaaaccatcagatctcgggagacttattcactatcatgagaacagcacgggaaagaccctcctctatgattcaattacctcccaccaggtccctcccacaacatgtaggaattgtgggaactacaattcaagatgacatttgggtggggacacagccaaaccatatcagggcgtcccagaaagggtatagggtctgagacccaagtcagcatgagaaagtatgcttctcatggtggcccagttgggtggaagtggcagccgggccgtctttccaccaggccactcaagtagcagctgagagacccctgccctggccagtccccgccctcccctcttgccactgcctctggttctgaacagatgggcaccctcatcttgtatttgtgattaatgtctaacaatgtagttttgtgagaagggtttgctgatacagccttgctgcagatgctgcgaactgtggcctggggcagaccttacctccagacacgccctgaggcaggggagggcactggcccgtagctggccgagagctctcgggttgcgcgacagggatacttttcagcggctgggtcgctatccaaagtgagaaaacgaggagggaccaggaggctgtccgcctcaagagatgtgggggccaggtccagttatctggggaagcagtaagcttctctgctgtttctaaccccaggcctcccctggtctaaggcagggcctcccagcctcggggcactttaaagatatctgggcctggccccatccccacagtctgactgagtgggtctggatagggcctgagcattggtgatttcctgggtgaaaggaggcccctcacagtctctggaagcttctctgtgttaggaaaagctctgggcttgactctgctttgaaagtcaagatccgcaaatcctctcagcctcagtttctccttcagcaagatgaaatggaaatgctgtacctacgtcccggggtggttgtgagacccaaaaaagacaatgttctggaaggttcctggtgcgttgcagtcctctaagaacctgagttagagccacgctgagtctcagcttcttggctccttctgtttcaaactcgtccatgtgatagctcaggaagggtaggcagggccctgccccctactcagaaaacaccatcctggtcctggggatccccgcagcattagtcccctgttttcccagtgtattgagaaaaattgctaacaagcagtggggcacaccaccagcctcctgggttcctttcagtttggggatttttggacattcccaggaatgtcttaaaaaacacttcaaaaaacattaacataaatatttttatcaaagcctgtattaaatggtctttcaagaaaatacagtaacaggtcaggcatggtggctcatgcctgtaaccccagcactttgggaggccaaggcaggcagatcacctgaaatcaggagttcaagaccaacctggccaacacagccaaatcccatctctacaaaaaatacaaaaattagctgggtgtggtggcacacacctgtagtcccagctacttgggaggccgaggcaggagaattgcttgatcccggaggcggaggttgcagtgagctgagatcgtgccactgcactccagcgtgggtgacaaggtgaatctttgtctcaaaaaaaaaaaaaaaaaaaagataaaatacagtatacagtaatagagaacaatccttttttcaaagtagtgaccccaaatgaacaaaatatgcatctagcttaaatgcgaacctggttttctctacgcccattcaagcccctgcaataggggcccttcaccccgcatccatggactcctaaaattatatggaaaatggctgtgtgtgagtgtggatggacatgtgcacacatatttttggctttaccagatgctcaaagagcctaggacccaaaaagggctgagaatgaccgtgtcggccacttcagggtcatcaggaattgctgtgcactgctcacttctccagtgaacactttctgcttctgtgtttcctggtatcctttgggactcctggctaggtcatgtgtttctctactttcaaaagggcttcagccaggcacgatggcatgagcctgtagtcccagttgctctggaggttaaggtgggaagattgcttgagcccaggaatttgaggccagcctgggcaagtagataggtagatgattgatagatagatagatagataaatagatggatagataagtcgctagacagtcatccatccacccatccacacataaaaaggcctttgtcatgtcatgttttgtggcccacctgccagtgttgcccacagttgctgcccctccaaactcatcagtcactggcaaacaggaggaatgtgtggctcatgtctgggcatcagtggctgtgggagacatccttgatcttctccagcttctccttccacattttcctttgcaatctggcaatatctattaaaataaaatgtgcatgccttttgacctaagagcttcacttctaggacccacttacacgtgtgtgacatgatgttcatacgggtttatttatctgaggttgttcatacacaccattgcctgtaatcactaaaggcgggagcagcctacacatccatccacagaggagtagatgccttttggtacatccgtggcgacggaatactaagcagcctgtgtatctatacactcacacgtgtttgtttatgtgtggaatatctctggagggtacacaagaaacttaaaatgatcactgtctctggggagggtacctgggtgcctgggaggcaggtcagggaaggagtgggcacaggtattaccaattggaagacaataaaaacaacagctcctggccaggcgcagtggctcacgcctgtaatggcagcactctgagaggctgaggcgggcagattgcttgcgtccaggagttcaagaccagcctgggcaacatagcaaaaccccgtttctattaaaaatacaaaaaattagccaggtgtggtggcatgcacctgtaatcccagctactcgggaggctgaggtgggagaatcacctgagcctgggaggtcaaggctgcagtgaggtgagattgtgccaccgcactctagcctgggcgatagagcaagaccctgtctcaaaaacaaacaaaaaacagtccctggcactctgggccaggcctggcagggcagttggcagggctggtctttctctggcacttcatctcaccctccctcccttcctcttcttgcagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaataaggtctccattcatggattccaaggacaagaaagtcatatagaatgtctattttttaagttctttcccacgcacccttagataatttagctcagaacaggaaatgatagtattaataaaagctggacatcaggattaacagctctctctggggccctgaaggtgagagttctcagacttgctcatttgcagttgcttctttgtgatgctggcaaaccatcctagtcccattcaaagggcaatacaaagccttgtggctgacctcacgatgcagcactcagtttgcaagaccggcaccagtgtatgcaaacctgagaaggttggggatgaggatatgggatctttcatccctggaaatttagtccagaggcctggggctggagcagaacaccaagccaatcagcttaatgaatggcttagattcctgctaggtttgcagagctgccttctttcctttggtaccttattatagattgaggagtatttctgctaaaccaagatagggataaccagatagcatcttcatagcaatgccacaaaggaaaacaaaaacaaaacagtaatccatcatattattccttagtaactatgccaaggtcatgatactgaatccttagattgtttcaaaatactacttttctttgctcttcctgatgtgtttgccaccgcaggcagatgtttaagtaaaacagattttaactgcagctacaaaagcagcaacaggccagcaaaagagaagtgctatctcagagagcatggctttcagagccacaagagacagcctcactggctgtttcagcttgactgccatgcaaagaagagagcagagggagaaccagccccacccacttattcatcttgtacaaaaaaaaagcacctaccagcctaggctacatagtgagacactatctccacaaaaaacccacgaaaactagctgggtatggtggcacatgcctacagtcccagctactggtaaggctgtggtgggaggatctcttgaggccaggaaggagatccaggctgcagtgagccaagattgcaccactgcactccagtctggacaatcgagcaagatcccatctcaaacaataaaaaaaaaaagcgtgtaacctcctcagaagaaagatgttataatctcaggcagca

SEQ ID NO: 33 - RNA, 22 nt, synthetic construct
  source 1..22: mol_type = other RNA
ttagagtgaggattaaaatgag

SEQ ID NO: 34 - DNA, 12001 bp, Rattus norvegicus
  source 1..12001: mol_type = genomic DNA
aggacaggtgaggtcctaggtggaggtacgtttttgggcttcatggggcagatgctgccctaggaagggtacaagacttctatagccctgctggggaccccattgtcctggaaggcggcccaactctctactcctgtggaatgggaagcccttgcttggctttccagcctccaaacaagataccactgcagctcagtttgagcgcttcaagagtagtaatcattgtctgagggaggccctttgtacccatggcctgctttctcggcaggtttacaatggactgaaatccaactctctaacttcctggaacccaaagaaagtctggctttggacaaaggccctttggctgggatttgtgcttatgtaattcttccgtatgcatgcaagtcatctggagaatactagtgagaataaggacaaccacagatatgtaagccttttaccttacatagacgtatttaacaatggtctaatacggccttaagtgtgatcagtaaggaagcggcagggtacgtatctgaaagatgacaaaacgtgactggagtgcctcaagaaaaaatgtgaactgtgtatctctcacagaagacacacctgggccggctatgtgcctgtttccttcagttggatatgtgaccatcactgcctagcgagttgtaagagtgacttctactcccaggccctcacaatatccgaccaactaacatttacccaggaggccctaccctctctgaaggcatgcaagtggttagcaattgctgagagatgaggctcttctgtggtgtagtgtctgtgtccacctatgctcccctaagtaacccaaataaacttgtttatctgccatactgaatttgggcagaattgtcatttcttcagtttgttcagtaccttttctgtctgtggtaaatagaagtaagactcccagggctggagagatggctcagcggttaagaacactgactggtcttccagaggtcctggagttcaattcccagcaaccacatggtggctcacaatcatctgtgatgggatccgatgccctcttctggtgtgtctgaagacatatttaaaagaaaagaaagaaataagactctccaggatagtcatagagcagagcccaggggtctacataagtaactgtatcccagtgtagccaatcagttcctcctgtttctctggcctagagttaggtttcggtattttgccatcctcagaattaaatcctgcctcctgagtgtagcagaacatgcagttttatgcatgagctcttgggagaccacagagatttcaattttaaaaagagacagttttcttttttagttgagaaacaactttaacggtccccagctccggaaaaaaaaaaaaaaagaaagaaacaactttaaaaagagacaattctgtttttagttaagaattctctctcttactgataccctttcttggctccagggactccccatatatctttctagacatttctgagaactcaagtaaatatatggtgatgtctccccacctttttttgtagtttgtaccttttgctcattccataccgtcttagaaaatatcttccttgaagcactatgtctcacccagtgcatggaggtttcacaaatgacttcatcaggcatcttgttctccagcgcatggctgtctgagaaccacttcaaacaggcaagaggatacagaatgttactatgcaagtaacaccagctggggatggtggggcagacgagcaattctagttattggggatgctgagacaggaggatctcaaatggaagtttagtccctatctctaaagttaaaagaaagccaggtacacgcctctaaccccagcaactgggaggcagaggcagaggcagggagagccctgtgagtttgaggtcagcctggtctgcataatgagttctgtgatagccaaaggtatacacggtgtgatatttttaaaaggaggtgtgtctaactggcagagcacatgtctgtcacgaggggtgtgtgtatgtcaaatccccagtaccagtaacaaaaacattagtgaagaataagtaacgtggtatgtgcccaggaattagaaacctgcagagaggggttggggatttagctcagtggtagagcgcttgcctaggaagcgcaaggccctgggttcgattccccagctccgaaaaaaagaacccccccccaaaaaaaaagaaacctgcagagaaaaaaaaaaaacctgcagagacacagaggtgtgtctggagatagaacatgggccttacacatattacaccgagcatccatcttggctcaccccaactttcacacagcaactgcggcgcgctgcaaagtcagtcgcaatccgcatttctagacagagcggcttcagaccttccaggcgcgcacgcaggcctcgccgaggttctcggtttccgccgcgactcggccgacgtcacagttagaagacaatagcgactttcccagctctgtctcgattctggaactttctcagtccgcaagctcctgaagctggcgctcccctcagccccgcccccaacgtgccccgcggccagggaacttcaggaagggtaggcagagaccgcggctagcgattggttccctgccaaggtgggagtggccaggcgcaggcatataaaagctccgcggcgctgggccctcgttttgcaccttcgtttcctgcggcggcttctgtcgtctccttgctttttgctctcccaggttccgaggccgccgcgcgtctcccggggaagcatggcgatgaaggccgtgtgcgtgctgaagggcgacggtccggtgcagggcgtcattcacttcgagcagaaggcaaggcccggggcgctgggcgcaggccgcggtgacgcggggcacctgtgcgggagcacgccacgcccccgccgcggcctgagcccgttaaatgctgagtcaccgcggccttgaggcaggggccgggcgcgggagagggaggccggggcgccgcggggccttccgggcgggtccctcttcgcgcccccgagtggccgggccggcccgagagagcgggcttggcatccgctatccctctggggctgctgcttttccggtgtccctgtcccacaggggctcagacccttgtggccaccggctgcatttgttgtaagaatatttgaacctggtggtgccaaaccggactaacgcagcaagcagaacgcatttgtggcattttaaagccaagccctggctatattaggtcagggtcgtgccgcaagggggaaagaaaagagatggccttgggcagttgttttgccaccaagagctccaagaaagagacctgactctggttgttgtctacgacagcgagtctctgagcacaatttgaaaagtatacagaaatattttcgaaactactgcagttctgcaaaaacacatgcgtcacaaggaagatatttgtgtggttaagagcgtgttcagagccttagggggttaacattgtactccttttaatcccgagagaaatatttgataaatgagcgttatgtactctctaaagtggtttacataaatgtgaggagaccgacaccatagtgaatccaagtgtttcctttatgaggagaactgataacgggaatttagagtttttcataactagtctcagtttcttggcatttaaatgtattttgttgttttcctgtgtaaattttttgtttttgtctttctcttcttcccacataattcactgtgagacagggcttttcccccaccctgagaaagctgaagactagctaggtctaccccagtgtccaccttcccagagcagcttgcagcattctttggtgacgctgccctttgtacccgatcaaacgatagttaagcattccaggttggcagctgtaacaacttgactatcaaaactgtttgatttaaactgttgccaacttttcaaaatcagtttttttctactcaaagttcctagtcccttattttgttgaaaacgttggagagttaaagtagaaaggtccggtatgagtgtccttgtttgttgcagttggttgcgtcttgccttttctccctgttgctacaatttctgaagtaatactaaatttgaattttggatgttcttttcttttttgttaagtagcaaattctctagatttggatgcctaatgagacttttttaaaaagtagctctggttagacccaaatggatccccacaggcagtaggacacaattattttctggctactggataaaattatgggaactgataaacatcactgaatgtggagtagaggtttctgggcagccaatgttctgaaagaatcaagcctgacacagtgcagtagccatccattccctagttctgacattgagctgcccccttttgttcctctgggtgcttttcaagtgctgttgagtccaggtgtctgcacacgtgcatctggaaacaagtgttagggaagatgggtagggagggagaggcctagagctaagcagctctagagtcaccctggaggaaatgggtctacttggatttggacataggtttgattttgttttgttttttgacttgtgccttttactgtgattcagaagtattaacacaaacttgatgtcttaatttttgtatttttttaaataaaggcaagcggtgaaccagttgtggtgtcaggacagattacaggattaactgaaggcgagcatgggttccatgtccatcaatatggggacaatacacaaggtaagtcttaatctatctctacctggctgactagtgagatgaatgggactgagtcaggaccaattactaaccatttaaaaccatcaatttttttctttttcttttagattaagttaaaataaccacttaggtcaacctcggaaaatagccacaaaagtattttagttagtatcgagtatttcttgactccttaagtgggaaggtgagggtaaattttcttaaatgtgattattatagcttgactttaatatacagaaacaaatacgcaccttccttattttggataatcctttgaggtgtttggagctgggggttgagggtgggtgctttaggcacagtgtctaaggacagctatgcacgagaggcatagtgggacagaagtgacaaaaactgaagattcaatataaatgcttagagtaaaattttttatatttgggattggaatcaagtcagaaaatatagtggcttacattgcatttagtgaacttttaccatattggagtaatgatctgtgtttgtttataatcttttaagagcctcattcatgttgctagagccttttcttcctttccctcttctcccctgctttcccctccccacatagtgcatgctagcctggaatctgtgctggagcttaaaccatttagtcttagatgtgcagggttaaaggccatcaacctgtgtgcaatgtacagtaatttggcctacagttacttatgtttgtgtttgacccattcggataattactaaagttcaatcaagttgctttgcctctggcctggtagctttggtttgttaagttccttccagaatcctgccctgtacctatttcttggtctgggtagaaattgtaaactatgtaagtcctatttcctgagttgttgttgctgttgagatgtcccttgtgaatgatgtcccttagcccacatgtacacctctaatgctgtttacacctggagttgagaagcacagcagccttgacacgtggggataatctaaaaatctgtctgccccaagtaaaagccagatggctgagctgtttggtggtgtaaggtcttgaaagataagtgtttttatcatgatcttaaaagcaaagatctttaaatgtgggactttaactttagaagtgccactaaaggtcgcttctgttccagtagaggaaggaaccagagctagacatgctgtgacactaccatgctcctggcacttggaaggctaaggtaggagggtcattaactgcagatatcatgggctgtggtagtgagaccctgtctcaaatctcaaaacaaacaaacatgacagtctagtgaaaaagcgggtagcttgaaaatgcaaggccatatagtccagctatttgtaccagggtgctgcttcctgtttgtatcactccagcacataccagctccatgtttgctgtgttggaagttgtaagaattccgatgtcattgcatacagaggtttacttcataatctgactgctggtttctggtaaataggctgtaccactgcaggacctcattttaatcctcactctaagaaacatggcggtccagcggatgaagagaggtgagcagcattctctctatgcatggtggtggagaggggtctgtggaaaacacctgaagacagaactgagtggtctcactgccttttcttttgtatgtttccattcacccaactcccacatccccaagtactggaatagtttatattgggtgaaggagctgacaaatgtggactcttaagtgatttagttttgtagcatttattgaagatgaactaatacaagtgccaaaaggaaccaatacagaaaatatcatggataacagtactatcagtcactagcaaagtaaatcattgtataatatactaatgcagataataaaaactagttgagattccgtttgtatgtgaaaccttaggaaagtcctacatttaaagaggggctagcttgcttttggaatggaggcctgggagcaaacctttgctaatcaggagctgacatccttttcgaaagtcctagactgtggctctctcctttaaactggaagagctatgtgtcaaggtatcctggctacctgttttgaaatttgtgtttccagacctttgtctggaaaagccatcatatttgatagtgtatgtgcactctttaatccactcatagcatttgacttcgatgtgaatttagctattgaactctattgatgtgaaatagatatcattgcttatccacctggtgctgttttaatgttaggcatgttggagacctgggcaatgtggctgctggaaaggacggtgtggccaatgtgtccattgaagatcgtgtgatctcactctcaggagagcattccatcattggccgtactatggtggtaagtttccatatagtagtagatgtaggatttcttctaacatagttatgtacctttccatgacttcgtggtggtggttaaactagttcctaaaagatcacataaattggtaagagttcagaataggaaaaaatattattttattggatgtaatagtaaagaattaatttgcctaggtcagttaagaacgctgttctgctgaagtgcggtagaaagctggttacatttgatcagactggatctgagttgaggatacaatagtctttagtttaaaacagctggattttcttgccatgattgcccccttacagttaatcatttcgttgagcttaaaatctgcgatggatgtcagtattcaagtctgcaggttatcgcttggttaccatatgggagccgtcttcccaagttaccctcgggagatgaatctggttcatgcagaacaccaagtagtaaaagctcttgcccacttcgggcagctaacttttcagtaggcacttcctttcagttgaccctttatccttagaattttcttcagccctattggtgaagcagaacaatcattcataaatgttctaaaaataaaatttaaaatcttgttgctaagtaaagatatttagaattgcctcttatgtgtaggcctatagttcactcaccaagagattttgatagagaaatttgtaagaatgactactgtacagtggggtgagggtgagggctaagatcagcatgtgcctggtagttatttgggtccttagtattcatctagaaatagccacgagcaaggaaacacttagtagtctgcttttagctgatagcataaaaattagcttattgatttactaatagatttgaacattttctaatatacatggtcctttgaagtattgctgggaagaagtgctaattacttgatcaccgaaacctaaatgttcttaattcttttcaaaggtccacgagaaacaagatgacttgggcaaaggtggaaatgaagaaagtacaaagactggaaatgctggaagccgcttggcttgtggtgtgattgggattgcccaataaacattccctatgtggtctgagtctcagactcatctgctgtcctgctaaactgtagaaaaaaaccaaaccattaaactgtaatcttaacagttgttaactgtgtgactcctttgacttgctctaaggacttgcagtgagaggtgactgacgatgtttggaggatgtgtagaacttcctgaatgtgtacaactcattgaactaaaatctgttgtttctgtgccagacctcactggtgttaagctgaaattctcattcaagcctctctctctctctgtgtgtgtctgtgtgtgtgtgtgtgtgtgtgtgtgtgtgtgtgtgtgtgtgtgtgtgtgtgtgtgtgtgtgagagagactgagacttatttagagcctcgagagatagagacttatttcaagcctattaatgtataccaaaaagacctaagctctatacactgagcatcaacaacagactcaatgaggctctcatagtatttaattttgaaagtgtttcatgtgataccatcaaaatgacgtgtggtagcccaaaccggatttgatcttagaaaattttctgccctttgttatcatcagaaattactgaaagctctctttaagattcagagtacctaaccttattttaaaatcgtattagagttagaagccatgatttaagataaagccctttagtaaacttgtataaaactcataaaaggcaaataggtagcctcagctagccaagtttaatacctcctctacctgccaagtgaagttggtaccacctgcttttttaaggttggcactcaggaaatacatagcactgggagatgagaccaagtggttctggcggttgtggctaaatcgacttttacagcctcagttaatgaaactgagtaccttaataaatacatcccaagaaagagagctttgtaagggaaatattaacctaaaaggctcgtgactatttggaacagttcaactgaaatatttctgacaaatttggtgtaaaccatagtcctcttaatttttcaaatacaaaacactgaaaattggaaaaaaataagtgtttccaggtttaaaaattttaagtttaaatttccaatttaagattgcttataaggaatgcatccgtatgtagttctgttgaagtttcaggtaaatcatacataacttatttccttaatgaagttttgatggggtgtgtgtgtgtgcgtgcgtgtgtgtgcgcctgactcaatgcagactaacttgggaagcatctggtggaatcacaaaccagtgtttatcagctcatttctcggtatttttagttggaaatacaagctgcaagtctgtccttggaatctggctttgaatagtcaggtgcatgttttagcatcttcccagaaatattctagtagggacttaaaaagcccaagtagggtcaccataaaaccagaacctggcatagctacagacacatttcttaccatcattaggaagatcttaaagagttaaatgtcaagtgggagcatcaaaaaggagcttctcagtactctaaggtctgatgaattatttttgtttagcctagcactttgtagttttaaaatcacattttcaggatgtgtgcaagagaggaaaacaagttcatgttcttccacctgttttgtccctgggatcacacaggccatcaggattggcagaaagtgtctttactggccaagtcatcttgtctttcaaatgtaatttgatgaaaggaacttccagactgtaaataagaggtgctcaacctgtgggttacaacccctttggaacttatatcattctgcttatcagatatttacaatccataatagccaaaattagttgtaaaataacaaaaataattttatggttagcggttggcacagcatgaactatattagagagtcacagcattgggaaggctgagaaccactgctgtaaataaatacctagcattaagtccctgactctagacaacactaccccaccttcttgggtttttttgtttttttgttttttgtttttttttgttttttttttttgacaggatttctctgtgtatagcttttactgtcctggaattcactgtgtagaccaggctggccttgtctcacaaaagatccaccctgcctttgcctcctgggggctgggattaaaggtgtgtaccaccacctggcaaaatttgtttgggtaaactttttttggtttcttgagacagggtctgtgtagccctggctgtcctgaacttgctctgtagaccaagctggtcttgaattcagagatgcacctgcctgtgccttcgctgggattaaaggtgtgcactacgacgacccactgggctcggacaacacttttatgtcagtgcttccaataaatactattagagtcttggtttggcttcacaagtagcagcaaaggttcagtgtacttcttgggctattgaagatggtaaagacaacaggcaaagcttacaagaaatgtcatactgtacttaagtctataaaaagctttctggttgcatatgttaacacgggcctcattttacaccttggaaatttcaatgggatgattataaacacgtcaaccttctaaataagtaatgcccaagcaatcaggtaatttatagtataatgtataatgttgggaatccaattatttcccctaacaaatctttacaaattaattagggcaacattaccacaaagccaagagggaaaaaagaactgacctgcagacacaaatttcttagttgaactttaagatcactaacctttaccttatggtcaaaatactaaaagtcaaaagaataaaactccaagcactggcaaatactacgtaagtgtctttctttccccatcaaagaagtctaatttagactccaacaattattaacagctcaatctttgatggttaacatctgtccaatcttaatgcagtgtatgaagaatagcacacattaaagtttgttacgaaaatagagtttattaaaaacatccctattgtttgaggagcttttcaccgttaccttttcttaaattaaaaaaaaaaaatagagagcacttctaattaggatttgtaaacttttaaaagtcaaaacttttaaaaagttacagcaaaaaaaggggtaatatatcttcagtattttttgttattttgtggctatttttaaatagaaggaaagcaatcaaattgctcacaatccccaccaactactgtggagtgatgtagcaggagcgaatattatacagcatctgtacacctcacgttctacacccaagtgtcactgtcacattctgtcaaatccagtctctagcgaggagcctctgctgctgagccagaatccttctcaggttcaacggatgaggtagcctcagcaggtgactcttcaggttttaccactgtggccatggcctcccccttattaagttctgaaacagtgtctgtgtttcccacttggctaaggggagcttcgacagctttgttaccacctgccctgtctgccacctcgtgcttctcctttcctctactttcttccttctctctacgggactctctatctctagaatccctgtctctgtcccgatgcccactagagcgtctacttctctcttccaagtctctgtgtctgtcccgctcagggctcctccttccccactctctcctttcacggttgctattatctctctcatcactacggctcccataccgctcccggtcattttccaccctacttccaaaagatcttcttccaaacctttcttggtctctacctgagttgaactgctgcctgttatcatttctaaactgctgtggctgctgctgagctggcgctggctgctgggatggcgctggctgctgctgtgtctgtgactgctgt

SEQ ID NO: 35 - DNA, 21 bp, synthetic construct
  source 1..21: mol_type = other DNA
cggatgaagagaggcatgttg

SEQ ID NO: 36 - DNA, 18 bp, synthetic construct
  source 1..18: mol_type = other DNA
ttggccacaccgtccttt

SEQ ID NO: 37 - DNA, 23 bp, synthetic construct
  source 1..23: mol_type = other DNA
agacctgggcaatgtggctgctg

SEQ ID NO: 38 - DNA, 25001 bp, Mus musculus
  source 1..25001: mol_type = genomic DNA
aagggtttctttggcttacacttttcttattgttgttcattattgaaggaagtcaggacaggaattcaaacaagtcaggatcaaggaggcaggagctgatgcagaggccatggagggatgttacttactagcttacaccccccccccccccccggcttgctcagctttctttttttttttttttttttttttttttttagatattttctaatttacatttcaaatgctaccctgaaagtcccctataccatccccccacactgctccccaacccacccactcctgcttcctggccctggcattcctctgtattgttgctcagctttcttatagaacccaggaccaccagcccaggaatgacaccacccatagtaggctgtgccttcctccactgatcaccaacggagaaatatgggcaacagcttgcccataggatgggtcactagttgggttggttactgttgggccattccctcagtctctgctccatcccccatctctacatttcttgtagacagaatcaattttgggtcaaaagttttgcaggcaggttgttgtccctcttacatttctcatggaggcatttcctcaacttatttcctctctctgatgtctccagctcatgtcaagttgacacagttgttcgggacccacatgaagaccaagctgttcatctgctacatgtgtggggaggcctaggtccaacccgtgtatgctgtggtagttcagtctctgagagccaaaacagtccaggttagttgactctattgatcttcctttggagttccatcccctttggggccctcaatccttcccacaactctaccatcagagtccccaagcttcatccactgtttggctgtgggtctctgcatccgtctgagtcagctgctgggtggagcctctcagaggacaggctttctagactcctgtctgcaagcatagcattaatagcatcagggattggtgcttgcccataggatggatcactagttgggttggttactgttggccattccctcagtctctgctccatcccccatctctacatttcttatagacagaatcaattttgggtcaaaagttttgcaggcaggttgttgtccctcttgctccactgaggttcctgcctagctacatgaggtagcctctttaggtttgatatcctcaatgctgtgaatcccaactaagatcaccccccattcattcctggtgtctcccctatctcaggtctcagatatgccttcaagatgccccccccccacctctccacctctgccagctgcagatttccattcattctcatggccatctggctatctctcctgttcctccccatacctggtcctgaacccccttcaccccactccccatcccctctcccacccagttcctttcctccatcttcctcctatgactcttttattccctcttctaaataagattcaagcatcctcgcttggacattccttcttatttagcttctttgggtctgtggagtggagcgtgagtattccaacttctaaggcacacagacaacctcagattctccagccctttgtgtgtgttgcttatttgaacaaacgggtgaaagaaaacacacaaagttggcgtgttgaaagagttagtcgatcttctggggtaggtttcagtacagagaccaaagggacattctcagacactagacacactatgcaaagacaggatgtcacatgacaaaggataacggcacaagtaaacatttaagcaacagtgttccataccggctcacgtagaaaaaggacaagactataggaaagaaagcaaacactccgccgaggactacagcaaagacagaaagtatctgcaggtacggcttcaaaaggagcatttctctcagcaacttatatctgttaatgccctgtcttctggaataagggcttagtttttatcagtagagagagattgatttttaagatgtatctgattttacattgtagatctccttagtcaccccctgtagtaaactaaggaaaacttccgtggagggagaggggaagattagtaactcgtagtgagtaagaattctctttcaagaaaaagattcaagagcaatacaaggcctagatatgaaggtgtgtgtgtgtgtgtgtgtgtgtgtgtgtgtgtgtcttaacagcctgttcagaatttagtaggtcacatacacttacaagtaatgaagacaatatattaatgaatttgcagtaattttttgttttagaaatagaaactgttgtaaggaggataatcattcagagctctttgatatgtatcactcacattcacatacatgcatacacacagagagagagaaagagagatacagagatagacagagagagaacccaaaaatgtaaagagaggaaatgagttgaaagaaaaaatgggaaactgggttagggaggggttcagatgacagtgactgggggctttcagagttgggagtgaggcagcgatggagagagggcagggaggagggagtgtccattgtgacctctgcagaactctgactagactgagcagctcacaccgtgttggagctgtcctaacactaccaaggggacgggggagaccccatgaacaccacctagggagttgctcctttcattctgtgtaaagtctgatgtcttcaaacttgttgtaaatttatactctgttctaaaaacagtgacattcttctctttgtaggatgacctcattacatcaggtgttatattttatcttttttgcctcagtttctagtggtaagttgctgtatttattttcccctaacataatattttttattacttgagagtttcatacaatgcaccctgatcacactcactttccattccttctaagttcaccctcccactcttgagccctgccagctcactccccccttcttgaaaaaaaatcatcaagtcaatcaatttgtgttgagaatatatactcgttgggaatgtgatcaaactcccaatggtcagccccttaaagaaaagtgagtctttttcctcccccacttctctccccactcccttacccagttggaagccatcaactgtgaagagttatacttcagcatctttactacaattttaaaggactctcttcagtatttaagtatggcttagaaatagctcattccttgacctgtaatgtaggaaacagcctaagtccacaaaaagaaattacacttcagaccccatatattgtggaataattccatgctgtgaactccagggaaggaaatagagtcgtttattttccagtgaaagctcccctttaatacatcaaagaaagaaagagatttaaatatagaattacaaagagtcttcacctatatcatctgaatgctagtaatatctgtctatagagttgcatctctatctacctacacaacacattgccatgattcctaggagcaagattagaaagagaagactcgactcacctcattgattattattccataagggattcagtctagtatctctctctgtctctgtctctctgtctctgtctctgtctctctctgtctccgtctctctctctctctctttctctctctctctctctctctcacgcacacacacacacacacacacacacacacacacactcaccaattcctgactgaaaatgttatagaaaaattaatgtgtggcttacacatttggttaatttaccccttgcaattatgcttccattctacattacatccagtaaatacattgcttaccattcagtagaatgaaatgggaagttacctcaccaatactgatcttaacaacttagtgtaagcacttcttaaaataatttatttatgttatattgaatgcctgagactgccattgacatattaagcatagttagttctttttggtgtgacacatgtgaacagtagcagatctaaaataaaataaacatatgtaacatattaaattatacagattatagcttaatttttctttgtgattagattgattttcaggttattccttcattatcaatgttttgaaatcccattgttatttgtactgtcttgttcagtactgttttgacatgttgttgttgttgttgttgttgttgttgtttgacacagagtttctatgtgtagccctggctgtcctggagcttgatttgtagaccaagctgacctcaaactcagagatctgcctgcctctacctccaagggctgggattgaaggtgtgcaccatcatcgctcggcagcctgtcttaacatcttaaacactgagttcaataactgtgtcgattcacaaggacattctgagaattataagacttttttgcttatgaatatatatatgcaaatgtaactgacaaaatattatccattgtggttgtatcacacttaaaaatctcagagccgagaaagttggggcaagatgattaaaagttcgaggacaggatgggctacataacaaggttctgtctcaaattggctataccaaaccgtccaacacatattttaaagaaaaataaatgggaggctagagagatggctcagtagttaagagcacttagtgctcttgcatgggatcagttcaattctcagcgcccatgttagatagttcacaacttcctatgactctaacttccaggaatacagcaccctcttctggcttctgtaggtacacacacacacacacacacacagacggcatacgtacatacatacatacatacatacatacatacatacatgcctacatacacatgtacatgcatacacaataaaaaagtttttaaaatctttttttttaaagaaagaaaattaaaagaccaattacattggcatattttggccaagtttgcttaattctaggaacaaggagttactttaatctaagaaaaacaatcaatggatgcaatgtagatccaaaggaagtgaaagaagagagtcctacagagatttgtcatttgtcttcctgctatcgggcagagaaccagcaagagagaaacgtgggcatttgaagcccactcagccgtgtcatagcacaagttgggtcttcaccaatggacagaaggttaaacaaaatataatatcacagtatgcacatgcaacacaaaacaggatattactcagcgtgtgaggaagaaaattctccctcatactggggcatagctgagccttcgtggttttatactcaacgaaatgagtcatttacaaatgaacacatgactgaacccctaacgtttggttcccagagatgccgatttcaggaaaacaaaagaccgaagagaactgaccaggggctaatgctaatgactatttaatgggtacaagttttcagttggaaaagctgaaggaattctagaatagtggtggaaattgtacctaaggtacatacttcattccacaactctagagacctgaaaagggccggagtgacaaactttatgtcatatatattttgccatagaagaacaaaattaaaataatctaacacatcgaagattttaaagattttcataataaaatagtttagcaaactcagaactttcccaatgactaagtagtactgtaaaacaacaacaactagaaaaacattcaaaccaaaagttttcaagaaatctcatatgtaatggtgactgaaatagtgtttccctgaggtactggggtaaggcaggagtatccacaacttggctcctgtgaataggaaacaaggaacagagagatgcaagcatcttcaaaaagttgtcataggatccatcatggacattccaggaggggttcagttctacttttagttttctgtgacatctcattacaggttttgattttttccccccatagcttcgccagcatggaaatttattcacaagtaccaagtcagggagtctgcctatgtgtccatcaatagatgaatgcaaagagcaaacactggatgtaggcacagtgcagggggcagggaggggtggcatgttctgcatttagctaaaatggttgttttagtttttactaattcttcaagaatgccatataccatattttgctaatttttacctcgactccccaaactccatccagaaccccccattctgtacccacctaactctgtgttctccctgtttcatttggccacagtgaggaatgaaatcgtgccatttgtgtaacaatggctgtgactgggcatcatcattattcagtgagataagtcagattgaagaagataaatatcccattttctcaaatttgtggattctagaccttatatggatacataaaatccagtgtgtgtgtgtgtgtgtgtgtgtgtgtgtgtgtgtgtgtgtgtgtgcttgagtgacacactctaggctccagagtctaggggaatgacagagactagaaggaaagagggaaacgaaaagatatgaagaataaatatagtcgaagcacatgatacatgcctatgatattatgatgtatacacacctatgcaggtgtcccatgaagccccctgctatgcaacgaatacgtgtcaataaatgtgttgagagactatctactgtcacttcccttaaacattatattagagatcctgtgtagcacagtagggaaggtcgaaggacctaagagtcaggagaaagatggaaattaactgactcttcccataaatgtggttatgtacatagaaaaattcaaagtaatgaatgcactgattatttcaggcagatcagcaaggagtttacaaactcgatggacccccgggagtggaggcacacacttgtaatctcatgactcaggaggcagagggcacatcagagaccctaactccagctcttccgttcaaaatcttcaatatacaaacagtagccaactctcgccagtactaggaactgtactgagattatgaaagtcaatattataggttaaatgattcaagtagtggctacatcctaatcaaaactgcacatttattaatcctttaaaaatcaattcaacttatcaaatcctggtgttgtgcttgataagaacattttaatgtgttatgatggcttaatagtaaaatgttgcatttcttagagttaacaggtaaactgtgaggtgggaatagactgccaattagctggaaacagacgcagtatggtttggtgggagacaacactgaacttggaaaaagaagtcggatctctatgtagctcctgggatacttgcctgtagtggagaaaaccatgttccctaccaagtctctgctgaattttggaaatggatctgtagtgaggttgctcttcctccccacaaatcccttccgaggccccatggacgcagcggagcctatccttggaataacttttctaaaggcatggactaaaaatatatagggttgctggggaaattaattatattgaaatatgagtagctaaagtggaaaatactcagtacagaattagttgtggctcggagttaacaccttacatgacattagtggcatgcccaaaactgaatagaaataaccaacaaattatcgacaaatcataatgatgttatatctcatcatcacatacatgacaaatactaatcatgttaattgattacattaataatacaggtatgtatgtaccacattttggttagggaatcaatgaaaaaccacatgaggtttttttgagctcgttggttatctaggtcctacaagcctcataggagggttttgagactgctatgcacctacaatcctctcaagacatgactgatggctgttacagtagtctatatttgagttaaattttattcttctggaagtacttccacgtgagtctggtaatgttttcaagttataaagagaaatgggctagcaacacctctccatgggcatgtatggctgcatgaagtaatcttttattcagtcactaggcaacctactgtgtgaattattagctatctcttctagcaacagagcacttttgaatttttatgcattttggctttttaaaaactaaagctgatgtaaggttttcgctaaatagaactcagtaagttttccctggtaaatgttggtcatgagaccatttttaatgttcgcatgacatcatacaagggcccctcagtcttggcttttagtttgtggtagtgtcaaaacaataaatgaacgtaaacacagcacacccttgctgaccccctagaggggtttcatggggaccgtgttcttcttgccggtcaacagctgagaaatcactgagttgctcctgcagccttcccaagacaatcactcataaatgtgacaacacggagggcccccatgttgtttgtgatttatagttccatctggggctgttcactgaagagcgttgcccgatctgatgtccgaggtcaggcatatttagcactgactttttaacaaaccatgtaacgaggtaagtgcccaccaacatctgcagtaatgtgcgcccgcgtgtttttttcttcacagaatgcgttactaaggtcttcaaagacatcagctttcaaggaggtgacctgagtactgttttcacaccgagcgccacatactgccgcttggtctgcactcaccacccacggtgcttgctcttcacgttcatggctgagtcatcttcggatgatcctaccaaatggtaacagtttctgtttctctgaagaggaactgatttccagtgccagttactcaccaatgcagattaccttacaaccccacttttgtcattttacaaaaggggatgttagtggctggaaagtgagtttccttccacggaaatactaaatattacaggaaggatagcgcttaggcatattggtggaaaaccgtaataactcctgggacctcaatcttaaagtcacgtttttgctcttcatccatggtttgtcttacattctttttctgtcgtggctattggctaacaatggtggggttatctagagcttccttctaacctttcaattagggggaaaatgtttaaaaagctactttaaaaattattacaacaattttctaccccctccaatatcctgtcatattatgtggctttctcggctcccatcagtggttcaataaggcatgcagacttttatataaaacttaaggcccttcattggggcaggcagactctcagctctcttctctaatcttagatcgtctgccaagtagctaatgatttatctcgtttccattcctttccctatctgcctctgaatctcccacctctgctttagttctctctctctctctctctctctctctctctctctctctctctctccctctctctctctctctctctctctctctctctctctctccccctctctctgtcccacccccagaagtcccacccttgtacttcccgtcccactttaggcaatatgagtgggtgaggaaggacaagcacttacaaatcagaagctggtgatgggccatagacatgacgataccaaacatctgccagaacttagctctttgccagtacagtaatcaacaattgcacaattgaaagtacagagacacgccttaatacaatataaggaaggtcatcgcaacactgtcatatttgctttacctacctgtatatggtcacagataaatactgaaagtgagtttaaaatatcatcgacctgcatatctaaatacttcaacatgaatttcccaataaggaaactgtaagaagcccaacaaagttaaacatacttgaaatgttaattttagctgttaattaaattctcatcaaatttcacaggttcctatggctgtttttccctctctatcttatttttaaaatattgcatccatttctgtctctatgctaagcttgaaattttaatatataacctttaatgcaaaattaagctgccaatatctttgttattgctgttttctcagatggttacatattgctgtcaacattaaatattcttctgaaatctagaacagagatcggcaagacaggaataaaactttaggctttgttgaaccaaatgcaaaaagaacattctgtgtttctaacactgttttagaatgtaaccatgtgtgggctggagggattgctcagtggttaagtttgctggctgctcttctaaagggccctaggtttgttaccccaacttacagagatttgcaatcattggtaactccagctctagatgccatcttctgcctttctcatgcatgtgatcacacacacacacacacacacacacacacacacagagagagagagagagagagagagagagagagagagagagagacagagagagacagagacagagacagagacagagacagagacagaggcagaggcagagagagagacagagagaggcagaggcacagactgagagagagtgtgtgtttaaaatttaaaatgtagccatgtaaaaaatagaaaaccatttctatctcaaggataacacaaaaatggcagattttaagcaacattttagcaataggttaaagtccttccattcttattcatgtcctgttggaaaggagggccgtgtgtgttcttttagatgtagggatgttgtaataaaaaacaaagtcaaatcttgctaattaaacatttgttttggcaggaaacccagtcgtttgtctccttctctttgaccttaggatcccctctttgaaagcatgaattcctgctgaataatgttattttcattttattttactattttattttttaaaaaagaacaggtttgcctgcatcctgaaggacagcgtcacagaaatattgccaatggtaaacatgacaggcgcgatctctggatattccttcaagcaatgccctcagcaattaagtagtaagattttttttatcaaatacaattaaaactagccattagagtatatacgtgtaatcgtttcagatacaggtttgtggctataaaataaaatactaccacctaggactaaacttcccaccaaggaaccatcttcctatgtagagtagaggacacagttttctcccttcctctccccataagcacactgtctcgctttaccatttcttagcctcataaaggcatgtcagcacctcagttcttattattgcacacgaaagccctcgttataaaagctaattcaaatcaagaggagaaatgcagactgaagtggcacatgttttaatagtggaacgggcacttttcagtaagttaaggggtgaaccagttggtcagtgcggtttgaggtcagaaagtcaaaattcaaattcacaagctttctatttgttaaaaaaattttttatttattttatatgcattggtgttttgcctccatgtgtgtttgcgtgagcgtgtcagatcccctgaaactagagttacacacagttgtgagctgccatgtgggtgttgggaattggacccaggacctctggtagagcagccagtggtttgaatcgctgagtcccctctccagtctcaaatgtacaagcttaaagcggttccagtctaagaggcacattggcttcctcggaatacctcctagtggaagtgggaatcacagctgagtgaaacaaaacatagaacttccacgggagagtgggcgagaagcgagcagtttccacgcgagagggccagcagcgaaagcagcgtactgtagttgcccctcgcttctggtgacgcctagattctccgcctttatttccagcttgcagcaaagatgtgtacgtgaacctagacatgaagggcatgaactataacagctctgtcgtgaagaatgctcgagaatgccaggagagatgcacagacgatgcccactgccagtttttcacatacgcaacagggtattttcccagtgtggaccatcggtgagtgagcgggagtccgagccgctggatataagcctgcccagggaaagaaaaccgctggttccgtaggtattttcatcaatttgaagcctaaacttctttttttaaaccccaagatatttgcataacaacaatcactgttttgtcatgaaaaggtcatagcgtgtctaacacacatttacgacatattcaaatttcagaactggaggatggctcggtgggtgcgtaaacacactgcttatgcagggaccctgagtttggatcccagcatgcacataaaagccagatatgggtgtgtatgcctttaaccccagtgcttgggacatgggcacgggacagagggaggatggttggagctcactggttagctccaggcctttattattattattattattaaatattgtctttatttacatttcaaatgttatctcctttccctgccccccccccaaccccctctcccatccccctcccatttctactaggttgtttacccacacaccaacccactcctgcctccctgccctggcattcccctacactggggcatagagccttcacaggaccaagggactctcctcccattgatgcctgacaagaacattctctttagctccaggcttagggagagatcctgtctggaggggaaaagacagagaggtcatagggcaggaactctaacaccccccccccacttggacttcctattctcacttgcatacccacacccccacaccccacacacaccatatacaatacatacagcgtggcctcacagattctgtgtatgttgtaaagcatacacaagcttaccctatctaacttcaaaaggcattacattttcacgcttgtggttctgagagctcggtttgcttggccatgctcttcccagtctaacaaatgtcctaacctaaaaatcatcaaaacttaaagtttgtttctctatctacctacatgtacagttctgtcccttacccaagacgaagtcattggaacccatgtgcaaagttttctcctgtttgatgtgggattccaaactcctccaaggaagaaatctgttatataaactaacgagagggaatgactaaatctgcatcttcagtttaaattgttgttagaaaagtatatgacttgttcttttacaaacattttaattttgtgtgtgtgtgtgtatgtttatgtatgtgtgtgcctgtgcgtttagacacacacgtgtgcagataactatatagccagaagagacatctctcaatctctcaaggttcatttttagacttaggaatctttgacattatctagatttgcacagtaaacaacaggtatgtggtagttggattttaacattggctattcctacttcactttccataattctgggggggaaaagatgaatgggaagtgaaacattaaagatgtcttttagaatgaataataaaaaaatggaaagtagccttctatggctctctttagctctcctcagaggacatgttttatgtcttagacttcacagaatgccaactgcaggctaggaaaatgttcctagggcttacacaaaagcgtatttggagggctaacgtgaacccgttcacacaaccacctcacaccacatcaggtcttctgtgttgtcctctcactgatgagataacattattcctgagtccacagagcctccctgtggttagaagagctgccaggactcatgcagccacctggctctgcaggtgaacactcctgggcagttcctctgttattacagtcatttcccccctgcggaccgtaacttcttatcctctcgtgtttttatacacttggaaagaacaatattttgccatttttgtttgtattcagtaaaatgtgtcttttgaagtacacccgaacggggacgccaaccacaataacgaagctcaatggcgtggtatctggattttcactgaagtcctgtggactttcaaacttgggtaactatcatttttctcaatgagatattggtaccattaagcctgagtgaagcagagactatgtgcaatgggtctaactttaaaaacagctgatggttatacatgaagcgaacccagtaaccttttactgtcttcaaagtgaaatggttcactatgtcctggaaagcatttccttcttaaatttcaaatttgttctttttataaacaacaacaacaacaacaacaacagcataaataaataaataaataaataaataaataaataaataaataaataaataaggtcttcggatgaactttccatttataatacaatttacaaaccctgtctgggatgtctatgattttgctggtgcccctgccttcctaacacagtttcttcccttggacagcttgtatcagggacattttccctaacacggtgctggcagaccttaacattgacagcgtggtggccccagatgcttttgtctgtcgtcgcatctgcacgcatcaccccacttgtttgttcttcacattcttttcccaagcatggccgaaagaatctcagaggtaaggcgttgtcattaagggtcatctggtctttttaaaaaaacggccaataaaaatgtgctgcacaatcaagataggaaacgtctaggcagcaggacacttctggactccttgagatagatttgaattgcggaaaggaatggtaccagcaggaggaaagactgggaccacggacaatagggcaaggttcaaaagtgttttgaaaagttcttagtgacattacaatttacagaacgcgacttggtgattcaagaagcaatgttaggatgaggttgctattaaatgcttctctgagctacctttatttgctatacttgtaccaagtggtctttctctttgctatattttatctgatttattcatacactccctttggtcctttagacatctttgtctccttaaaacctctgaaagtggattaccaagcacacgcattacaaagagccacgccctttcgggcttcagtctccagcactgcaggcacagtgtcccaggtaaacaatgcaggctgtccctctctctgagctccacagccccaaggaactggatggctgtgaaggctacacacttcaaacctggcgtgtgctttgttgtctagtattctgccatccgtccttttacaacgacactgatttcttgggagaagagctggacatcgtcgatgtgaaaggccaagaaacctgtcagaaaacgtgtaccaataacgcccgctgccagttctttacctactatccatcgcacagactgtgcaatgagaggaagtaaggcacaagttaggtggatgctcttggagcatctccttgtaggatgagttttgcttacagagttttgttttcagccgcaggggcagatgttacctaaagctttcctccaatggatctccaacgagaatacttcatgggaggggaggcatctctggatactcactgaggctgtgcaaaatggataatggtgaatacttgaaaaaatacaactgaaggggaatagtcaacctaacgttgctagtctactacacgaggctagtctacaacaaccatagagagatggagacagcagcacaaggaggttgaggcaggagaatcagaaatttaataccagattggattaaaaggcaaaatcctgtataaaaaatgacaacaaaatagacatggaagagagaacaaagttaacaaatttggaggttttcccttacatatatgtatgtcatatatatatatatacttatatatatatgtatatgtgtatgaatatgtatatgtatatatgtcatttcaagtggcatttcctgtagagacagacccagagggccaatttttgttttcaagaagtgttttttttaattatcagagattaaactattaaacagtccattaaataaattattcattttcttcccacttaatatttcagtgagccatgattagatgctatgatatatgatatgatatatacacacacacatatatataatatctctcatatatatatatatatatatatatatatatatatatatatatatatacatacacatacatacacacatgcatatatataattccagatgttaagctatcctgtaaattgtgatgagatttcatcaataaagtgtgaccctaattactcctcgtgaaagtttcaaaagtataaaacctttttcatcagatcatttgctattctagaaggtgactctatccttagtttcagaggacctgatttacagcacattgagatgttttatcccaacaactgcagtgccctaaacagaaaacatgccttcctagaattcactggtttgatagcaatctctgggtggcctgagcctcttaagacagttaattaagttatagttcatacacactgtgttttgctcatgataaacttacctaataagaaggaacattcaagacaagtattgtcttaattctacttcttcatggtagaaggggcaactagaaagacggctaagtcatgtgagcatgctttaaaaactgggatccagaacagatagcttgatacactgaagattacatttctcacccacttctgccttcattatgtttgtctctgttgaatttatagcctggtctgtacaggtgacagaatggatcagttgtagattgacagaagagaaaatgtggagggtaataaacctgtctgccttctcatgcatagagaagtggttacactgtacaatattgggctacaatacttacctttatgcaagagagaagatcgaactcagttgtttttcgtatttactctgttgttggtctctaatgtaacttgacttcctaaagacacctagcaatggacaccactaaaagaagtatttcttcatccccaatgcaaagttgagcactataagttttcagcattctgttcaagttgatggagcagacatcgagatagaactttttctgaaggcttgcattgggcttactgataatgtgtcctacttactgcttgcctgttaactttctaaaggttacctttctgctgatggactgaaaggtttctgagggatttctcagaagcctttcaggacgagggacattgaagcctaggtaactgctaaccacactctctctgttgtagtgtgcacaactaaaatcaaccccagagtggtaggaggagctgcgtctgttcacggtgagtggccatggcaggtgactctgcacatcagccagggacacctgtgtggaggctccatcattggaaaccaatggatactgacagcagctcattgtttctctgggtgagtattattgctattctcctgggattgccatcatgaaggtgaaatctgggactatcataagagtcaataaacactttgaaaatgtaaatgatcctgtttcctaaattaattctctctgtgtgggcagggacgatggtgtagaagagaccagtcctcatcatttggccacaatagaacaggggcaggagcagagcagattgcccacctctgccttttcattcaaacgcaaattatttccattgtcttcctgatggtgcctggtggcgaagcacagggccagagtgaagcttacaatcctcagctctctgaatcctggttaccctagtctctctttctctgcctccgaatgcttagagttcagcacacaccatcagaccagatccccgcacttagcattgcttttcgatattggccaaatgtgaacatcttagccggggaagtgtgtatctcgaggaaattcggttgagtgaaacctttctgtgctacttttagtgcctctgttgcttccagacacaggtttagaggctaatcgttttgttaatttttttccatgcatggatgcattatgcacataattcaatgctacacttgagatcaatagtcccctttgcaagcacatatgaaaaaacacagaaagtcccagtgacttttctttaaattctgcccaagacaaggttgagactaatacccaactctcctgagcttggagatgtgctggggagtagaaagaccatttatttaaagtgtccaatattagtgcaagaactaatccagtgatttcactgtagaggaaatatgtgactaaaagttttgagaataaaatcactttttttaccacctaaaggtagaaacagacacagagtataaataactgtgaaacacaaatatttggaaattgcctagtgatagatttttttttccattctgtttgttccttaggatagagacacctaaaaagctgcgtgtctacggtggcattgtaaatcaatcagaaataaatgaagggactgctttcttcagggttcaagaaatgataattcatgatcagtatacgacagcagaaagtgggtatgatattgccctgttaaaactggaatcagccatgaattacacaggtatatatatagagagagagagttttaggtgacctagataaaacattcacgttaggagactcacagtctcatctatggggtctaatcaacagacagacaaggaaggtctgaaaagatggcctcactctgttgagacagagagtttgccttagaatactagattagcgatccatacttatctccttgtactaaggtcaaatctaagtggatcaaggaacttcacataaaaccagagacactgaaacttatagaggagaaagtggggaaaagccttgaagatatgggcacaggggaaaaattcctgaacagaacagcaatggcttgtgctgtaagattgagaattgacaaatgggacctaatgaaactccaaagtttctgcaaggcaaaagataccgtcaataagacaaaaagaccaccaacagattgggaaaggatctttacctatcccaaatcagataggggactaatatccaacatatataaagaactcaagaaggtggacttcagaaaatcaaataaccccattaaaaaatggggctcagaactgaacaaagaattctcacctgaggaataccgaatggcagagaagcacctgaaaaaatgttcaacatccttaatcatcagggaaatgcaaatcaaaacaaccctgagattccacctcacaccagtcagaatggctaagatcaaaaattcaggtgacagcagatgctgtcgtggatgtggagaaagaggaacactcctccattgttggtgggattgcaggcttgtacaaccactctggaaatcagtctggcggttcctcagaaaattggacatagtactaccggaggatccagcaatacctctcctgggcatatatccagaagatgccccaactggtaagaaggacacgtgctccactatgttcatagcagccttatttataatagccagaagctggaaagaacccagatgcccctcaacagaggaatggatacagaaaatgtggtacatctacacaatggagtactactcagctattaaaaagaatgaatttatgaaattcctaggcaaatggatggacctggagggcatcatcctgagtgaggtaacacattcacaaaggaactcacacaatatgtactcactgataagtggatattagcccaaaacctaggatacccaagatataagatacaacttgctaaacacatgaaactcaagaaaaatgaagactgaagtgtggacactatgcccctccttagaagtgggaacaaaacacccatggaaggagctacagagacaaagtttggagctgagacgaaaggatagaccatgtagagactgccatatccagggatccaccccataatcagcatccaaacgctgacacctttgcatacactagcaagattttatcgaaaggacccagatgtagctgtctcttgtgagactatgtcggggcctagcaaacacagaagtggatgctcacagtcagctaatggatggatcacagggctctcaatggaggagctagagaaagtacccaaggagctaaagggatctgcaacccaataggtggaacaacattatgaactaaccagtaccctggagctcttgactctagctgcatatgtatcaaaagatagcctagtcggccatcactggaaagagaggcccattggacatgcaaactttatatgccccagttcaggggaacgccagggccaaaaaggaggagagggtgggtaggggagtgggggtgggtgggtatgggggacttttggtatagcattggaaatgtaaatgagctaaatacctaataaaaatggaaaaaaaattaaaaaaaaagaatactagattagactttgagagtgcagacggagagacttgccttcaggcttcttcaggtacatagaatgacatcgttttataaaataccgaagcaataagaataatgattgatattttgctttagtaacaaagggcttgacagcacagttgagcaaatgctacagaatattaaaccacattaaaaatgaaaggtggtgtaaagagggcctgtcttaccctttcccttgtcccttctgcttgggacatttcatatgtgccactgaacacatgacatgaaacacatgagaaaaagataacgaatatcataaagaaagagagcatgattaatgtcaggtgagataacacccctttctgggacaaatgattggctttctttctgcttcggtcagcttttcgctactgtgacaaaatgcctgatgtaaccaccctgtaagaaagaaaggtgatatgggctctcttgatgtcagaggcttcagcctgcagttgtgtgtgtgtgtgtggggggggggggtccactgctttgtagcccttggtaagaaatgtgcagaggagcaaattttcacctcctggatgctgggaagcattaatgaagggacggggctcctgatagccccttcaaaggcccaccctcaatgacgtcacttccttcctacaccttcccccccaacacacacacacacacacacacacacacacacacacacacacattctccattatctctaagtagtgtcacaggctgggatggagtctttgaacatgggcctttggggagacacctcagatccaaaccgtagtggcctctgatgactaaactgtgattttcaaaattagattttcagcggccaatatgcctgccttccaaaggagatagaaacgcagtgcacacagaatgctgggtgactggatgggggtacacagcactaagaggtaacaaaccatgccttctatctctgctttattctgaagtcaaagaacagagcttaaccattgcctctgttttctatctagtcatatggcccaaacgtgagtcaagtcacctactcaataacaggaagactgataacaaagatcaatacatctgatcagaaacgttaaatatgattaaacccctctaaagaccattttaactggagacttttagtttgggacctaacactctatgtaaaagttctagcctggtttctaattattttgtctgaaaagaaattctacttagtgtcagttaattttgaacttaataacattaatgaaattatgtacacaatagtagaaacaatgtcttctttatactccatacttacaaaaattacttatgaatcaagcttagtaataccacccccccccaggatttgtatgtacaattttggcttttaaatataattgtatataaacctatagtaattattcctctaaaacactaatatgacccttttcaggtgaagtacaaagtactcttcagaaagccaaggttccattggtgtcaaatgaagaatgtcagacaagatacagaagacacaaaataaccaataagatgatctgtgcaggctacaaagaaggagggaaggatacgtgcaaggtaaggcagtctcaagcaatcagtcatgccagattgaagtgagagcttaatgcatttgtacaaaccactgtaccattgagcagtgtccgagtgtgcttcctgttgctgtgataaaacactgaccaaacacaactcagggaaggaaagggtttatcaagcttacaggttacacagtccaccatagcggaaagtcaaggtaggcaggaactgcagtagagacggtggaggagtgctacttcctggcttctgtttagtcttgtgttccctacttttcttttgtgacaatgtggttaacaattagcagtggagaaagttccccacagtccaggctgatggcagaggtgcctcagctgtgttccctcttcccaggtgtgtgaggttgacaactcagattagccatcgcaagcagatcacttggtgggtttattttaggtaaactaaactctacaggaggaaggaaagctggataaaggagaacaattggatgtttggatgcttgtgagagggccagaatattatgtaaaattgctgtgagcaatacttacttaactcaggaaatgcctaccatgatcccggcatgtgtcttctttttctccccctttgacagggagattctggagggcccctgtcctgcaaatacaatggggtctggcacttggtgggcatcacaagctggggtgaaggctgtggtcagaaggagagaccgggggtctacacgaacgtggccaagtacgtggactggattctggagaaaactcaaacagtctgaaagagttcaactggtatcactttgtggccctggaagattattccatagaaatgagcttgacgtctctgatgaagacactgggatactgactcttccactgtaaccaattgaatggccttgatgtacgtaagaacacccagaaagaaaactattattttcagaattcctgatctgggagaaccactggttgttttctgcatccagctactactcaaggaaacaaatacagcaaggagattttaaaaataaaaacacatcagatatataaggaaaatatcaagtaagggtgctgtctgcctttttagtctctgtgacaaatacctaaagtagttcacaaaaggaaaaatttcttttgcacacctttcctcaggtttcagcctacgatctggttggctggccccattgctttagcctgaggtgaggcagaaccatatatccataggaggctgtggagaaggagtctgctcagttcatggtaggcaggaagcaaatggaaacaggaatgtattggggacacgaatggtccttcaagaatatactgtcaatcatttacttcttccagagacatcctgctccctaacctccctttccttcccagataacacctctgtcctagctggccccaagagatcaggtagaaaggcagaggaaaccatataaagagttgttaagtgcaaaatcaaaaccagaaggaatgcagacaggagctcaaaatgtccatttataagaatctttttttttctctgcctatatgaatccccctcctgtataaaggactgactcaattcagtgatgggttttgagaagtctgtttgtgtgtgtgtgtgtgtgtgtgcgcgcgcgcgtgtgtgtgtgtctgtctgtctgtgcacattcatgtttaggtatgtgcaggtacctggtgggggcgatcaaccttgcattatttctcatgtgccatctaccctagcttttcaaagacagagtgtcttactgggattggagacttgggctaagctagctatctagcaagtccagggaccattctgtctctacctccccaaactgaaattaagaagacatgccatggtgcttaatttaaacctctctctgtctttgtctctgtctctgtctctgtctctgtctctgtctctgtctcgctctctctccttcctccttctctccctcccactctctctttgggtgtgcacgtgcgcatgtgcaagtcatagtgtgtgtgcagggcagtggaccatcttcaggagtcaatgctctccttccatcatgtaaggtccagggatggacattaggtgttcagatttggtgacaaatgtctgtaccttcttagccacgtcacaagccagctgttccgatttcctacagatgctgggaatcaaatctgagtcctcggggttgcctggcaagcatcattactgactgagctctccagtggccttgtcagtcttctctctgcattttcccaaactggcttggacaagcaccattgcaggtgttaagtgcacacttcctaatttccacatgggccgagtataggaggagcaattttccaggaagtggtcccttgaagacacaccgtactgatttgcttgcctcggaaagtatctcagcgtagcctgcactctttttgcagtgttaggggaaagtacaggtggatggagataaggaagacaagccaaaacctaccaagatctgccagtgagtgggagtttacaaagctgagtaatgaatgtgctggacggaaatgtgtgttgaaatcgtacatactacgggggggggggggggggtggataatttgggagcaaatgtggtttcaatagaggctgcagcctcctcaaacagttctctgtattctgagtacctgacttttgtcctcacatggggcaataatgtagtatttggactttgtccccgtacttttcagtcagcgttgataactatacaagttgtccaaatgaaaagtatttattgtgcccaattatgtcagagtgtcttgttgagcttggggaactgaagcgccagccaataaattatgaaggtttcataaggttttctgttgatttagtacgaaccgaagagaggagctgcacaaaatctatactttcaaacaaagatgaccatgacacaaagggttctaagaaatgacaacgaagaagagttagcagaagctaagagagtggcatggaaaggaagtggccccaagcaagacaaagcaaagacagcaaacaagcaaaagccagagatcgatgtcactgaaatggcacgagcaggctggattcaaaatgcttctagagtaagacagaattgacatcaaatggggtcacaacttcacaacccatgaacaagcagcgccttttataacctatttattacatttcacataggaaatcttttataacctatttattacatttcacataggaaattgaggaggcattgctgtcttctctgagaagtatttaaggaatgttttcgtcttaattttttttcagaacaagtgcaacatcttaattctgaatatctagtacctagaaaatgctatgagctataaggaataagaaattacgctgagcagattcacatctccacaccaacaagctgcgaatctgtatactttctggcacttttctcacttaatcttctctctcctggagctagctc

SEQ ID NO: 39 - DNA, 16 bp, synthetic construct
  source 1..16: mol_type = other DNA
ctgtttgagttttctc

SEQ ID NO: 40 - DNA, 16 bp, synthetic construct
  source 1..16: mol_type = other DNA
  modified_base 5: mod_base = OTHER; note = uracil
ctgtttgagttttctc

SEQ ID NO: 41 - DNA, 20 bp, synthetic construct
  source 1..20: mol_type = other DNA
acatgacaggcgcgatctct

SEQ ID NO: 42 - DNA, 25 bp, synthetic construct
  source 1..25: mol_type = other DNA
tctaggttcacgtacacatctttgc

SEQ ID NO: 43 - DNA, 26 bp, synthetic construct
  source 1..26: mol_type = other DNA
ttccttcaagcaatgccctcagcaat

SEQ ID NO: 44 - DNA, 34001 bp, Mus musculus
  source 1..34001: mol_type = genomic DNA
tgcagctagagacacgagctccgggggtactggttagttcatattgttgtttcacctatagggttgcagacccctttagttctttgggtattttctctagctccccatgtggattatcttaagagggtttcaatagtcacataagaaagtctctcattttaggggtgaagaccactttaaaaaaattcaaatggcttgctgaaagccacagacactagagaaataattatgtttagggtaaagtcggcttgtcctcttgtgaaactgtgtccaacggagacaagctgaggaattatgggcaatgataacatgttaaatgttgagctaaatgtattaaaaagcgcaaaaaaacaggtgtggtgggggaacagaggctagaagtctccctgaaaaaaatcaagtcttatttcctacttcccccgtgtttggtctggggaagaacaatagtgtttgcttaagcatctcattctgttttcaaaaccttgcatggttgacttgggaactgatagagaaaatgaagcaaacaatactctgtaatcaacattaacatcaaagctagctctctccactgtggtccctgacccaaccacgttaatctcccagggtcttgttaaaactgtaggcttgggagagaagtggttgtggtgaggggtaccttggagtaattctaagtctcttggggttaagcctggaagattggtgctttatgtattaagagtcctggggatccgaaagtagaaaaataatgatttttgttagtgactcagtatggttcttacttcaacacctcctcttaagggaggaagtagtgtgtgcatgtagctggaattttaaataataacagttcaggtgtgtgagtatttcattagacagtcagcttccatcctttatgcatcctttaagcaggttggtttagacaggtgcacaataaacgccaggctattaaacacagggactccaatatttaaaccaattcttttgaattgtctggctagaagcacatccagtctgataaagtagacaaagcacgtggagtcttgaaaagttcgacaccccttcggtcctcagatgactattatttagcaattgagagcactatactccttcttttgcccacacctataaggagatggacatggcacgtacatcccagcacttttgacagctttaaatggtgtttattacgagcaggtatggatgctgggaacagtgcttataccgagtcctggaaatgcttattaaacccaggagatacaaggagtctgccattaacctctctgtaactcaagagtagttatcaggagccaatgagggagaacaagagaattgaaacacgtgggaatatcggaccaccaggtagcgggtgtgtgtgtggggggaggcagccctagggagctctcaagccgaggcccctcggggtccccgcggcacgtggcgggctcgggccggagcgtcgcggcggtgggggaggggagcggcatgagccctggaggaggggtctagactgcccgggattcgtttgagcaggcgcggagtgcgcaggctcggcccaggcgcaaccagtgcgcgtaagaccgaggggtgaacctcactgctgcgcgtccgggtctctgagcaaggggctaaagtccgcttgtgcgcacctccggactgtgtgtgactacccgtagaaacactactgtcgccctggccggtggcgtccactccgtcccggtcgctccctcccgaccgtagttgtggtgcccccctccgcgcagccgcagtggtcgctcccgcctcgcagccccgccgcccggaggcctacccctgccgagtgtcgcgcgtcggcgccgctgcctatcccgctctgtgccctacggccctcccggcacccgccgccgtcccgctccgggctgcccttgctccgagggctgcagcaccggccgtccccgtcagccctcttgtctggccgccgggccgcggcgggcgggagcagccggaggaggagccgcagccgggaggcggcggcctgagcccatggcgtacagtcaaggaggcggcaagaagaaagtgtgctactactatgatggtgagtggccgaggagcctgcagccgggatgcggggagggcgcggcggggtgtcgggggtggcagcccgcggggacaccaacacggcctacccagcggtggcgtctcggggacgcccgccttcgctcctctcgggtcctctcgggcacacgcaggtttagggtgcactgcctctgaccggtctcccgaggaaccccagttggcaccctgggcctgactgcacaaagctggcgtcctggagctccgccccacgtccctgtcccctcccccgctctcccggtccccccttctcgcccccgatctgtccgcgctgcgcttccagcctcacccgggaccccggcacccgggtggggctgcggaggagttactgctggccggtggccaagttcgcagagcggcgctgtctcgctggtgttttgcgtggactacatccctcgcctttgttcgcgttcttcgagctccactttctcagggttcaaatggaagggctgcacttcctcggtgacaattaaggggttttgagttggaagtaggtaggtcgttgggaagtggtaggagagaggaagcgtgcagttgttcttgtttgatgggtcctggcatgtctctatgggaggttattacctcccccttctttgttgctctttgttcgatctgctataataccctgctctggggggttaggaaagaacaccggcatcttgcttaggaactcttctactgtggtgaagaccttgctgcaggaaactttgagaagtggactaaatattcacaatgcagtgaacggtacaaggtgactgtaaggattttctaatttggagtataagtagtaatagtctctgggttcagttacaccaaatggaatggtgccctgcctatacagatttcacccaagacatcaatagataaaccattgagagaagtaataaggaaatttgacagaactttcgaagctcattgattgaacgcttgatctccacaaagcaatatgaaattgggagcgttttctgtagttgtattgaagtcacagtgtttggagaggtagattagagaactaggctgaatgaaacacttaaacttgttaaaatagtcgtgcattttcttggtttgacagaatcttttaatacaagctttaaataaaatagccacctgtcattggctggaaaaccaaaaaccttacagaaagctaggccaaaatggaaaaggctgttttgttttttgagacaggctctaagttgacctggctgtcctgaaactcactgtgtaaatgaggctctaactcatagagatccacctgcctctgcctcccagatcctgggattaaagatatgtgccaccatggcctggcttaattggaagtttgagaggactcaaactgtattgcatgtttatttgaagttacaccatcatggagagcagagatgggagagcatgtcggtttaaacatgagttacattatgacgttggggtcatagcaaagccctgacctgcattctctgatccattggaaattacagcctttctaaagtaaagtcctgctcttgttagagtcaggggaaaggttgagctgttggggaacagctcagtctcttcagctgtcagcttctgtcatcagtgatgaactctaagtcccattaactttcaccaggcctgatggagaaccagagaggactctctgtcttgaacagctccttcctggcttcatgcgtagggatgagcctttgctgaggagtgggatagacattggtcatggagataaggtgtggagggaggcatttttcagtcatgtgattttcgacttatgcccatgcagctgctttacctagttgtgagtgaccaactgtataattgatggtggaaatctgctgagttatctatctgtggttcttacttgacttgtttttagaatgtacttaacttatttttattctgactatttgcatttgggggacgctaacaaaaattgaagatatataaattggtgtcatagttaagctaattaacatgtcatagcatgctagactacagagaattctctgcagagaattcccttcacgtctgacttgattacagaaaccctcttcttgttagtttataaaggtagcagcaatctttaattgtctttattaaattctaaacaagaggagcaagtaaagtgacttttgtaccaagaaagtgcatgactttcagacgcatgcagggttgggttggtggagaacccggccagtaggattggggctcgaatagtatactagtcttatgttagtgcttttaacttggttggtaggggaggagatcaggaaaggaggaggttaacggggcactaggcacccagagagcctcttgacaactcatgtgagtgttttcctgctaagactgtgtgcttagtgaagaatacttctgaagattgaatatggttttctccgtgtgtgctgcagtcttcagaattgtacttgtagggtctctgtgccactgatctgtgaactttcagataggagcgtatgaactatccctaaacctcacaagagaaatcagagttgttcttagtggactgtgactactgactggtcatcataactcccagaaacacatttcagaatgttggctaatctcttactaaactaaaagacaaagatcggtaaatgatataaagctaattatgtgtctacactgagatcctttctgacctctttcccgtttttctcaaagtgtaattgagcagatggggctacatttagttctaattgcaaggctcaggcttttaatactttatctagaaatataaactttgcctggtttagagtgaagcttgggaccaatcatgtagtgccttctaatgtatagtgtataacagaattgaatgtatgcataatttatgtaattagacaccaattgctttatgctttgtttattttctctgatagagataatcagaatcaaaatgtttggagacatagtccagaggaaagcacgtggcaacaaaaatatagaatataacaattactataattatatttcccagtgtactcctaagtttcttaacagatacaagagtactacagagtggccatcggttgtcagatttattttacttttatttaaaatgtgtgtgcttcttgtgtatgcctggtgcccttgatgtttaggagtggttgtgagctaccacgtagatgctggaaaccaaatttgggtcttctggaagagcaataaatgctcttaagctactctccagcctgtccgttcccactataacagtaggcaggaggatggttaggcttctgccacttatagatgaaaacaggtggacacttcatattctccacatgggttttctttccccacataggtttctgttttaatgtctaagaaagattcgtacctagaatacaaagattttatttttcttttgcagagttttgttcttctgtccctaaaaaaatgtattaataattggtttttttcttagcaaaattaaatgttagtaaattcttaaaacacatggagttggtgaacaaaaggaaaatttcttacattagcaaaatgaaatgtgaagtcattaagtaacggtttgggcttttttaaggccgccattgctatggataatgacaccgagatttatttctgtttagtaataaatgcctaggccttaagctaggcctactcccaactagctcatcactcaattatctcatttatacttctcagtttcctacaaccaacttcctccgagtccgaatagggaataccccacgcctaattctgagttcctctctcttccagatgtcccaccttactatcctgccttttgctgtaagccataggctttttattttaatctgtcaggaggtgccttaggcagttggggaaggatagagacacatcttcacacagtgtaccgaaacatcaccctaacaccattacattgaagttctactctcaccatgttggttagtcggtctcactctggtttcatggtttatgcttgtacatgaagtcagtttggggttcagaaacataagattgttttaagtccctgctttgtcccatatagacaaagattacttaagaaggaaatgggtgtttgtaacagagtaaaagaggaatggagtaataaaacaagcatctctaggaagcatatgcgccagtcttaaaacagtcctccctgtgaaacaacaaaggccaagtccttggaatgtgttgtacccaaaagaagggcaacccagagatagggatgctgggctgagccccaaggggccaggcacagcaagcttggttgaaagagagtttgactcttagagggagtgtcttggagcttttaaagtggaggctagtcaataagagaattggtgagaagtagaatgtaggctagaggaaagtgttaaagtgaaaggaaatggttgtctttagatagaatcgggtacatttgagagtggtttataaaccaagaaaaaaatacatcaaaactacttgttggtggcttggacgagggattagggctgggagttgtgatttctggactggtcatgagatggacaacatactgtccattgagattatatggaatcaagggctctatattggctattatgtatttgagatcttgatttagatcaaatgaaaatacttccaaaaattacctggaacccagggagaagtagtatttcttagctggtaagtgataaatactgacgtgtttgttgaaggtaatggtatggagagacacagactccggaaattgttcctgagaagtcataggatgaagtcactcttggtgtactgcttggccagtgacaagctcttgacttatgaggaaagtgaagaaaagctgagatttaaaaggtttggaggaagtgagcttagagaaggaacacaatggaggacagaattgtggagatccagactccacagtgtttcaaggaggagggagggtctaacagtgttaaaaccttcacagaactaatgttgtgtggcagacagaaattgcacagtagtctttttattcttgattttttcccctccctcgagcccccccacacacactacttttggcagcttctctatgtacagtgtgaaagtacatttcacagtcctgaaatttacaatgatctgaaattttgttcactcagaatcaaaagcttatcctgaaattaacgaaattacaatttattgtctttattccactaactgtgataattacacatttttgctttagaaatacctttgattatagaatgttgtcccagttttagtacattatatatttttatcttgagtcaaacttttctggatattcttaattggtaaaactatatttaagtatttgaacaaaaaatgggatcacattttaaatgcccaaattatgtaactgacagagactataggtacatgaaattaagaaattatgtaactgacagagactataggtacatgaaattaagaaattatgtaactgacagagactataggtacatgaaattaagaaattatgtaactggcagagactataggtacatgaaattaagaaattatgtaactgacagagactataggtacatgaaattaagaaaagaagagcagtgtgaggtttgatcttgctgtgcattgcaatactaagtgcaggccccaagatctggagtctgcacacagacctgagtaacagtatgtggccttgccttcaagttatttctgactggtaaataaagatgcctacagccaatagctgagcagaagagttgtatgtggggcttaggattcctattgtaaatataaaggttgtgtgtatcttttatctggaaactaaatggtcaaagccagggtagaaacgccaggttgggattaagcgttttaacaacagggctggatggcttagcagtttaaaaccctggctgctctttcagaggacctgggttggattcccagcactcacagggttgttcacaggggtcattcactacagccacaggggatctgatgctgtctcctgccctccatggccaccagacacagtcatatgtatggcaaaatcaccatgcacattaaataaaatttaaaaagaatcagttcagaaaacattttcttaaaaaagaaaaaggcagaaggttttttagtaagaaatagatgacttcaaagtcgccatgtataaaactaatagtgaacaggaaacattagaaatgaagattcttccctctcctcctcccagaatcacctaccctctgctgctagagtgctggtttaaaggtgccttgcccacaaaaatggttcttgtttgtttttgaaattataatatgatcacaccattttcttccttcctttactccctccaacccctcctgtgtagccttccacctgctctctttcaaatccatacccttggatttctttaactgttgatacatataaatactcctaaatacataaatacagtgttactatatgtttttagagctggctatttgttattggataaccaatgggttggtcattacctggaggagattctttgtcctatccttagtccttagttgtctgtagttctttgtctaggcttgaggcctcctgaccttcactaacgtgtctgttaatgaccttgttcaggtcatgtttagccagtcatgttggtgagactttgtggatgttgcttctgacatgtctcactgacaggcttacagtaaactccttgttcttcaagctctttaaaccctctctctcttccccaatgccctctgaccctaaggtgtaggggttgtattgtagatgtgtcacttggtactgggctccaaaactctgcatttcaattgtgcttttctgtaatggtctctgctacaaggagaacttttcctttataggggtgaggactacaggctcctgtgtttacaaggacaattatgtagaatgtagatcgggatgctgctgcttttgtaaagtagcaattgcatattttattcaaagatggatgacttcagtagtcttgagttggctaggtctccaatactaggcatgatttccctcttgctgaatggatctgaaatccaattagaaagctgttggttactgtaaaggtctgcgtgccaccactccacatttatgctgccatggtggttgttactgtggttcagaggtgtcataactgcgtaggactattggttgcttccctcctctggagccttgcatgactcctttttataatatgaagggtagtcctcaagaaggattaggctctcagttctgtccagctcaggggcttcttgggccctgcatctcaagtgcatgatgtcttcagcaatatgcagttacctctagggggcaaccaagggcaatagcgtataagattttgggagtctcttggatagtcctgaccagcaactccaaagacggctgcgtagttttttgtttgtttgtttgtttgttttgttttgttttgtttttgtggataatggctcctggagggagcctgtgtctatttatacacagtcttatgtgtattataggtacagtagggcaatggcatgattgtgcttgatccttgagacatcctcactgttcctctaccgtcctcattccttgtcctgtatttgtcttcctccctagttagaagccccactccattccccttacatttcctttccttccccttccttctccccctttgcgttcccgtcgctccccttttgtgcagaggaattaaaccaaaagcttgtacatgctagagaaatgttccaccactgtatatctcaagcctttggagggattcggaaaaattcatattatggctcagctgccctcgtgaatatgtgtcttttaggctaaatgaatattcttactgagaataaggcctcaaaattatgacagaagtttgtcgaaagctgtatatattaatataacgttaggagtctcatagttagaaggtaactcccttcataaattaggtaagccatccattttgttcatattcgtcaaatgaacaaattcgatgctgagcacaagcatgtatactgtattctttcctcattcgccattgtcctgctattacattgctgtgccagtgacaatacaaaagaatattcactgcctgtgctctcttcttcctaaatctgaatgtagctcctatctgctagttgtataattttggctacattatttaacatgcttctcggcagttataaagttgtggtacatcctagagttgagaaataaaagctgatgctgagtactaggaaaatgttcttgctgttacttctcaaacactacaacttaaagttggctgcatagggagaacatctggaaggattaggggaggggaaaataggacaaaaatatatttaaatttaaagttaaataataatataataaagaaagtttctacttaggtcaacaagatgtattgttttctggttctgaagttttcatttacctttgaaaaactagttagcattctgagtgctcctaaatttgtaaatcattttgtgaaaaaattgaactaaataaatcagaggtactataccaacagattcatactgtttgaaggcaggttttgtaaacctgaatgttcagctgggtctggtggcaaaagccagtggtctctcagtggaatcatgaattcaatgcctgcctgggacacatagctagaattttgtctcaagggggaacaaaagcaaatgttttctggattattgtcaagtagatagatagtatgaaaatttctggatttttgattgcccctatataagtgaaaaggtactatgagaggagagtttgaaatggggatgtttgtgtttgagagctggtcttgctgtgttgtctggccagcctggagtcctgtttgcaggtagataaggcttgccttgatcttacagagactcctgcttctgtttccctcagtgttagattaaaggtgtgcactaccataccctgcttaagctttgtccttataagggcagatatatgaagtgtgggggctgtcttttgttgtataaacctgtgctgaaacagtaagatctgcaggctgttaaagtcaggtcaactgtcctaacaaattatgaatatttgattttaaactataacattaatagtaatttctcatttcttgcttgataaggccattgtaaaattattccttatagggcaggagaaattactcagcagttaaaagcattggcagctcctactaaggacccagggttgtgttcctagcatatagccatctgtaactccatttccaggggatctgacacatcgtgaccaccacaggcaccaggtgtcaatgcaggctgcctatgtatatataggcaagctcgcaggcacataaaagtagatagtgtccctcaccttagaaaaagtaatacataagttttctaagcttgttgacaagctttcttattgtctaaaagtattttgtggttgaaaatcagattttggcattattctgtgtgttgttttaagacattggcatctgtcttacttagtttgggttttactgctgtgaacagacaccatgacctaggcaactcttacaaatgcaaacatttatttggggctagcttagtttcagaggtttagctcattatcatggtgggaagcatggcagcatgcaggtagacatagtgctggaagagggttctacatccttattcaaaggcagcagcaggagactccttcacaggcagccaggaggagggccttttccataccaggtagagcctgagcataggaggcctcaaaacccacctccatagtgacacactttctctaacaaggccccacctcctaataatgccacttcccatgggccaagcatattcaaaccaccacagcatctaagatgttttaatgcacaggctactactgtgtagtcctgagaaatgaagacaagagtgtctttattaccctgaaaaatgctgtgactctcccacttgtggacactgaacatttaagcccttccataattccagctcgactgtaaggtatttctacaggattccaataagttatccaagaagcactactagctgacaaattagatcctactccgtacttcaagaatacttcttatgtatctaaatttacaaaatgaacaacaacaacaacaaaaatccactaaaaatggatttcaccttaaggaaccaaaccagggaaattggaaaactaaaagtcagaaaaactttctctctccacaaagaatgtgaatcctaggcatgtataaaatctgcataatatattagatttctaatgtaatttgaatgttacaaaaacaactcttgtttaaatataaattttttgatgttgggtacatgtcagtggtggtatttaataagcatcttttctcttttaggtgatattggcaattattattatggccagggtcatcccatgaagcctcatagaatccggatgactcataacttgctgctaaattatggtttataccgaaaaatggaaatatatgtaagtactagttggcactgtgtttttaaactggtatttgaaagctcttcttaggctgctgtgggagatgcatgtggtagatgaaaagatctgaacgaacacagacaggtcttgtggttgtgtccctcagagaacgcttaaggaattggagtagtcctctcttccctctgatcatttcaaacaccaaatatttttgtgacaatactgttaagttgcctgtgctgctagaaacaattgtcctggggttggtgagatgacttaataggtagaggtgcttgccaccaactggatggcctgtttaccttccccagaactctcacagtagaaggagagacctaattcccccaaattgttttctaattccacgtgggcatgaggacacacctccaataataagtaaccacagtgtaattttaaaaagaaataaaagagaattgacctgaattattgcaagagtttggtagaaaattaatcataaatatttatttggttaacatacatatgaaatgttgccaatgataacaaggtaagtaaaaaatggtgtaagatacacagccacctaaaaagtcttcgaaacatgggcaggagcatacataaaaagtgcaaggtaggataaggcttaactaactactccaggagaagagggctagacagtgttataggaagcatttctttaagatacaggtattttagagaaatgggaagacttgtggaagaagtctgtggtaagtgaagctgagctcttcagagcagaagttagaataaacgaaggctaagggagatctcaagctggtgttcttagaaacctttgatgaaagtcagtgggtagacagcagcgaggctatgctgggaaagagcctgaacaaacaggagtagcctcgcttggagaggggctgccggctgcctgctgcctgccacaaatgtgtgcatttgaattagcattgtaacttgctactcaggtggatctggttcatttagagagactgatcgtggaaacttacacatatcatttgataattctcatttaacaactgatacttccaacatcctggtgtttttcttttcagaggcctcataaagccactgctgaagaaatgactaaataccacagcgatgagtatatcaagtttctacgatcaataagaccagataatatgtctgagtacagtaagcagatgcagagatgtacgttataaataattattttactagtgctgaatgtaaatgaatctttttaaagtttctgatcagagttgcctcaataggtatttttcctcataatttaaaatattaatataattattaattcagaaggtcattgaaccaatattaatgttactttagaaaacaaacctatttaaatttgttcttttactttttattttgcacccatgaaaagtttgggattggtagggagatcacagagttacaagagaaaaatatatttctttctttttttttttttttaataatttttttgagacagggtctgactttgtaaccagctgtcctggagattgctaatgcagatcaggctggcctcaaactcccagagttccacctgcttctctcttgcattaacaatcctccatttcaccttctgtgtactccagtcacattgttctgtaatgtaaagatgttgccttttgttggcttctttgtgtttctttttattcttctacatgtttttgattataggtgaaagtgtattgttattggtagtcaattgtattaactggtagtcaagtgtattaaagaaatattgccaagtatcctgaaactgtgggctgctgtcttttgaagcttaataccggagcccatttcctcagtgagaggtggatttagttcagtgatgacgaatgataaggatttctcaagttcactgaacaccatatttccctaccgtatgttatcgcatttggttgactcaagactggaataggacggacggatgctattacatctattgttttttgggttttttatttgggatttacacagtaatgggtagccagcccttgggtaaatgacaggatttgtttaaaccattatagtgcttgtttatattaaattctatttgagcataaaaaactaaaactttttttatagtatctgtaggtctgtaaaacccctgccctcttgctaacgtgtgatctgagaagtgaagaatgactagggatgggccacacacacttctgttgtgttttttcctctcagcacagggagccaaccatgtcatgaccaagtgacatgtcatctgtcttatgttccatgaaactgattcattcatggctgcttctgaagtcagtgttagccacagaaaaaaaacaaagtaacttaatattttgatactcactaaaacatgtttcggagtcagggacactgtgtgtgagggtcagtaagatgagttagaaaggggttgcagccacttaatctttaactcactatcatatttaaagagaaaactagatttgtgcctattttcatataggcttttgatttatgttgttggtgatggtggtggttgggtagagtgacttcacaaatttcctaataagatttggtaatggaattacatagataacaatttaattatcttgtaaaataagcattatattataaaattataacttttataatactacctacattgaagtactactccctgtgaattttaaaattcataggtaatattttaaaattacaaatttcacattgctaccatgaaatattatattattacctatgattgcctgaaacaaatatttttaatagtttataagaaaaagccttcaatgacttgataatagattgactttaatgaagttcaccttccacagttaacgtcggagaagattgtccggtgtttgatggactctttgagttttgtcagctctccacgggtggttcagttggtgagtatcctaaatcagtcagcctcaagaggatctgaaggggttagagtgtctgtaggttttgtctaaagcggaagttgtaatggtagtaaggttgtgttggcttgattttgctgtatcacccaggtgggcctccgtctcagccacctctgtggctacctcccaaatgcttgcttacagacatgtgctatcattccaacttaatgagtacccaagctgtcttgtagttcactgtggggcccgggctgaccttgatattgtggaaatcctcccctggagcctctcaatcattgggattagaggcttggaatattatgatattgctattcactcttctgttaatgtaacaaaacctgcggtaacttttaaaagaatggtttatcttggttcacagtttgggagcttaactggccctgtccttggggactgtgatagcacattatggtatgtgcatttgggagaaggccagtttacctcatggccgctgaatggagaaaagaggattccagtatccctttcaagcaagatcagatctccagtaaactaaaatcagatcttggctcttagagagtccacccccttccagtggtaccatactggtgactaaatgttccctggggttcttagaaggcatttcaggttcaaagtagcaagtataattgtctttaaaatgccagtgtttaacagtttttacttgaacacagtctttaagcttgtgtctttttactcagagacctttttctcctcttctttcttccgccatttttactaagactttgcatgtgcactgtgatttagttgggtaaactgtaggaaaatggttatctgaggaaagcttaggctccgaagttataatcctttgcttttgaatgccaaaccttttgtgcttacacatggcatatttaatagggcttcgcttatatgtattttgtgtaacctgattttttaaaattctgagtattttataaataacatagcatgtatctccttcttagctggggctgtgaaattaaaccggcaacaaactgatatggctgtcaattgggctggaggactacatcatgccaagaagtcagaagcatcagggttctgctatgttaatgatattgtgcttgccatcctcgaattacttaagtaagttaattcaaactgaattttccctgtgatcagatctcttaattgaaagaaaaaaatgattttaaagactatcaaataaatggtaatagattaatgctgagtcttccagggtttgttgtgagcccctgcagaagtgtgagaaatagaccactacagtgggagagcgagagggcagacagtgccgtgcctgagactgccggtaaatggtctctgctcatttaggtttgcagtcgtctaagctgattaaaaatggctgctagagatccggaaatgtgaatgctaaagtaacttgaagtcaagcctttgcaacttgtgttataagaagtttgtctggccacttagtagcgtggctgaccccagttctctcatccatcctctacacagacacacagaacacatagataatagacacacagtgatgcccaactgctgaaggggttttggaagttatccttcactgctttttcagaagtgtgaaggtccttaggagtgtggacacttgtgaaactagcttattttccactgttagctataatgctgcagtgagttgtatttcagctttgactgcagctgtgttctgtgggctttgagagtgatgctcttgccccgcatgtacatcccaagagttaacttcctgaccttaaataaacagcagctaagtgctgtcagtgtaacatatctgactcccccaaccgacagaaaccggaaatctccttcattgacttgaagcttcttccactggcttcagttctagagatctgctgctttttccttcatagcttagtcttgagtaagcccagttctgtacagttcttcacctggtaaatctgaacatgatttgattggtttggtaagcagctctctttgctcatctaaacacattgggcatttgatcatgtttgattttgaactttatgaagaagttctgctatgatgaagcccgctcgctgtttgcactattttagattttcttagtgttgcttttctgtgccttgagaacataaatgcctgggtttgatcatgccagcttacgttgtatgagcacagttacagcatctttaaataataaacaaaatgttttaagtcagccacagtgacaagataatcatttactaccttaaatggtgaatttaaatttcattttgtatgtattggtgttttgcctgcgtgtgtgtctatgcaccacatgtgtgcagtgcccatggaggacagaagaggccggtagatccccaggaactgctgtaagagatggttgtgagtcaccatgtgggtgctggggatttaacctggattctctagaaggcagccagtgctcctaactactagtccaactttccaacaccagtagctttttttaaatacagtttttttctatttgcactttgacatagggatggaactgatgtcattctatggtggagtgactacagaagctcatttgagtagttgctgtgactggtggtgtacagagcatcagggaatgatgaaggcacttgtgatctgtcacagtcatgaggtcttagtatgctgtagatgatgtgtaaaatgtgctcatctcgctgtaaaaggtgtttatggatatggacattggactgtctcagaactgtcatgttataaaatactctgagttttgttttattcttggaccattggaaaccggggagtggggtggcacaccggcatgccagaggcaggtaggtttgagatgagcctggatgagcagctatcaaaataaccacagggaaaaggaaacaacctatagttctgctcactccaaagctgttgaaagaataaaaataataacagcactatgttggttacagttgcccatagattattttataatgctgaaatttttttaattgatttattttcactttatgtcatttgtgttttgtttacacatgtgtctgtgtatgtgttggattacctggaactggagttacaggtaactgtgagcctgctgtgtgggtgctggggactgaacctgcggcctctggaagagcagccagtactgccaactgttgagccatctccagccctccctccctccctccatgttgagttgtgttgtttgtttgtttgagatagggttcctctgtgtagccatgggtctcctagaattttctctgtagaccaggctgacctcagactcaagagacccacctgcctgtacctcctgatggctgtgatgaaaggtgtatgtcactaagcccaacaattctgaaattgtgtaatgtagcccttggcataccctttaggtgtgagtacatagataatgtctgcccgtttttaaagtgtaaagacaagggtagacctcagagtacagtactcgattgtcttggtagcccgaggagacgtgacgtgctggggtctggatgctgagactgcgggggaagaagggtgagcattcgctgtaagatgaaggacctgcttccacattccggcttgcgctttcctttcaggtatcatcagagagtcttatatattgacatagacatccaccatggtgatggtgttgaggaagctttttatacaacagatcgcgtgatgaccgtctcattccataaatatggggaatactttcctggaacaggagacttgagggtaagactgagttctgtcagaataaatataagaagagcataggaggttgctaatttctggaagagccatgttgtcttagtcattttgtttgtgtatttgtgatgggacctcggcatctggtgtgtatggttttttttgtttgtttccggagatgtggcctcaccttagttgagcagttgttgggatcattgcttactgttaggatttgtcacatgtggacccactgaatgctgcttgttgttcagctgtgattactttgaatatgtagaaacgagggatcagaagtggtctcaactcagttaggtgtaagtgctgatcctaaaaagtttgctgtcctctccaggttgcataggttgtcttgggtagcaggggtggggtcacaatgggaatggacagtgttctataggtgtggggtacagattatcacactgtcttgtggtaggttgggagaggtcaggcagtgtctttatctgagatcacaggcatagagtcccagggtgtcttattaggtggtgttgaagactgattttgggctccctacctggggttgtgttctcaggactccatgttgccccagagtggcgcagtggatgtagaagctctgaggatccctccctacctatgggtctgggtcacagggctcctcactttgagaatggtggataagtgggctctacttttacatcttgtaaaattgtttcatgcctcaaactgaggtttgtaagaactttttagtttattttgtgtggtggggaggagcacatgtgtgccctgttacatgtgcaggagtcctcctgccatgggtggatcctggggatcaaaccgtggggatctcacttgttgggctttatctacccaggtatttcgccagcccaaactacagtgcttgtgtgttatctaaaaactgccttagagcttaaaacgtacttctacaacctagaatacccaagacacaatttacaaaactcatgaaactcaagaagaaggaagaccaaagtgtggacactttgttccttcttagaagggggaataaaatacccacggaaagagtttcagagacaaaattcagagcaaagactgaaggcatggccatccagagactgccccacctggggatccattccataaacaaccaccatacccagacattattacatataccaacaagattttgctgacaggaccctgatatagctgtctcctgtgaggctatgccagtccctggcaaatacagaagtggatgctcacagtcatctataggatggaacacagggcccccaatggaggagctagagaaagcacccaaggagctgaaggggtctgcaactctataggaggaacaacaatatgaactaaccagtacccccagagctcgtgtctctagctgcatatgtagcagaagaaggcctagtcggccatcactgggaggagaggcccttggtcttgggaagattatatgccccagtacaggggaatgccagtgacaggaagcaggaaatgggagtgggtgggtagggaagcagagggaggggggaggatataaggaatttttggagaggaaactaggaaaggggatagcatttgaaatgtaaatgaagaaaacatttaattttttcagaattgtgtgttacttggtcttttaatcattttaaaatatgtcagactttttgttatgaaatagtcttctaagatctacttttgtgttttaggatattggtgctggaaagggaaaatactatgctgtcaattttcccatgagagatggtatagatgatgaatcatatggacaaatttttaagcctgtaagtactgctttcagaaataaaatgggagttgtaaatatccttagatactaatgtgtcttattctgtggctagatcatctcaaaagtgatggagatgtaccagcctagcgcggtggtgctgcagtgtggcgcagactccctgtctggggacaggcttggttgtttcaatctaactgtcaaaggtaagcagttcacgttccccctggtgtggtgtttctcctccccaagaacttcccataaaagttttcattgctgagggctggagagatggttaagaacacatgttccttttgtagaagacttgggttggatcccagctcctacatgttggctcaaaatctagcactctcttctgatttgcatgcatgtggtacgcaccatacatgtttaaaaacatatttttaatttctcattgttaccttttgcttgccaactcgatgccaaactctatatttgaatttttagtggatttttatttgttgttgttgtttgtttttgttatgtggctgactagttaatttagttccccaagtcttacatgttatcatatttatgtttatgtatttatccatgagtagtttgttgccatgtcagcgccagcagtttttaatcagttttttcagaagacctgtaccttgttgtctgattcagttgctgttacagagtatgaggatttagcttggcgcactttattctatcatggttactctcttccttacccaaccctaagaacttcggtcgctgtggctagtgctcagcagcagtggtttttagcttagcagtcttctctactggcaagactcacttttttttcttctggatttttttgtttgcttagtttccatattccttattactaaactatgaatttctaatgtattctgctcagctatctcaggtgctctgtcactgagctcctcctgttcggacttagtgtcctcatgggtttaatgggacagagagctctctgcttctgctttattatgtctcctgatcagtgggcactgtatctaacttgctggtggaacacataagtgtcttcatgaggaaagaagcccaagtgtaaatgtgtaaggtggtgttgaaattctcaagtccttacgaagagccgaagtccacatgctgaatacaagtctcatattgctgttatgggatgaacttcgagggtttcagaatgaagcaaagttgcatgtggcagcgcatgcttttagactcagcattagaacagcagagatgggtccaggccagctaaggctttatatatagtaaaaccttgtctcataaagaaaaagataaataaaataaatctttttaataaagttaatgtttggattagaaatgacttagcatgcacattgcatgttttactttagactttctgtgtgaactttgaggtagtaagtaacatcttgctttgagttctcttacagaacttttaagataattaaatatagatgttaagaacttctcatgattgtactctgcagaccagcctcagcactgtggcatgctctggtactctccttcgtatctagttgggtcatacaaaattagttggccactctcctggaggtcagcattcagttaacagcactttcatgtgggccatagaatgttcatagatatctcgtgtttccgagcagataagacttaaccggaagtcatcagccaccaggtagttcctttctttttaggagctatatactgttagtgttctcttttgtaactgaaagtttataatgcttgtatttaaaagtagtagctttcattataaactgcatttgactctttatagtacatctgcattgtgtttcaacaggtagaagttcatttgtgaatttgcaattccattttcagacttagaatacactgctgcgactggcttcaactacattgtctcaggcctagtgaagccaagcagccagctgctgtccaccttgcctccttaattatttcttcttgtggtgttcttttccgtgcttcctcttacgggttatagccttcattgtataaaacccctttgtagtaatttttcctgctgttgggtggtagaatgctggtcttgtctgggctctttcattgtactctgaagttaacttctgtccttaaaaatgtccaagagacactgttgtagtagaagatgcctggttgataatgtacatgaagaaaacagagaaggcactataaactctcactaggtaatgaggatttttttttcttttgtggcattttctttttttccaaatattttaagctttgttatagaaaagttacaaaaacaaacaacaaaaataaaatcacacactgaaattaaccagacccaaagattcaatgacttttgtgcttagagcagccagcgtctcaataccaggtgaaagcccactgcaaaaggaaatgactttgttttttaaaaaaaaaaaaaatcttttttttaataaggctaaaatccatgaatctctgctgtcatcattgagggactttctttctttaatggttgaattatgtgcctgaatggcaccagaagttactggaaacttgtaacttgttcctgtgtgaggcttgtaatctgttttgttttttttttgttttttttttttttttttttggtttttcgagaaagggtttttctgtgtagccccggctgtcctggaactcactctgtagaccaggctggcctcgcactcagagatccgcctgcctctgctgggtgctgggattaaaggcggaggtttgtaatcttaaataaattaagaaaattatccacaataccagcaataaggatactctgtggtcaaactttttggtattgcttttcgaaattaagccacacatgtctgtgcgggagaaacaaaatttagcatttgtttattgtgttctctcccttatgtgatctctcatgaacgtcttctatggtagtaaaatacatgtgtgcgtattttaacacttagtacaagttattagttggatatacacccccccaggccccccaccctcgtcatcatcccctgtcattgttcctccctgccccccccacccctacccccccccccccaccttgtgttgaggtgcctgccttgtagtcatgctgtatagactggcctcccaattctgatccacgtaccttagcctcccaagagctgggattgctggtatgtgccactctgcctggctgtctacagacattttttggttgagtaaaattgtttgtatttatcctttttaaggtcctattttaaattgctaaagaatacatatttcattgttacccaaatcttctttgtctaaggtcatgctaaatgtgtagaagtagtgaaaacttttaacttgccattgctgatgctcggtggaggaggctacacaatccggaatgttgcccgatgttggacatatgagactgcagttgcccttgattgtgaaattcccaatggtaggtgttcaggttgcagtatctagaagaacatctgctatgtacaaatggatgcatgggagagtctactgccacacccctgaaatgtgtgatctcttctgatggatgagtggttagattgcaaatctgtgtgagagcattccatgtgcactttcaagctttcctttgggaatagttcttttatctggattcggattatgtttctgagattatacggaagctaagcttttaatgtgtaacttgttttttcattgtttttaatagagttgccatataatgattactttgagtattttggaccagacttcaaactgcatattagtccttcaaacatgacaaaccagaacactccagaatatatggaaaagataaagtaagaaatcacttcggcttaatgaaacttcaggaggctatagaaggtcaaataaaggaagttggtttagcatatacatcagatacttcctaaccttaggctattcctgttttttaatctcttatattaatacaaatatgtaacctttgtaaatagaaacattcttattagatcaaatgctttatgtctacagaatgtagaaacattgatcagaacgggctgtgtcctctctcccatagaccagttgtatgacatttataagtacacctcattgtcaaatagaggtgaaaaggtacatgtttgtgtgctgtgggttcagtcagctaatatatgcagtgatctgatgcttagatagtgtcccattcagtggttagtaaatgaaagctcgttcttgtttagttcctcctgtgtataacagaaactttacatacagtggattttgtctaataaattgtgtcatttagacagcgtttatttgaaaatctacgtatgttaccacatgcacctggtgttcaaatgcaagctattccagaggatgctgttcatgaagacagtggagatgaggatggagaagacccggacaaaagaatttccagtaagaaaacccttgctatgtcttcttgcatttttcttatgtgtcaaaataagacttaaaattgaaggtacacagggaatggttcacagcacatgttgtgattttccttctctccattttaattacatatagatttagctccctgatgtctcaaagcctgaattaatatcaccagtttcattttgtgtgactacacagacatggctgtgtccagaaagtaggcacttgatatatctatctatctatctgtcttacctatgcaatgattgtagaacctgtggctattatcaaatttataaaatcttttgtgtatcagttcgagcatcagacaaacggatagcttgcgatgaagagttttcagattctgaggatgaaggtgaaggaggtcgtaggaatgttgctgatcataagaaaggagcaaagaaggctaggattgaagaagacaagaaggagacagaggacaagaagacaggtcggtttatgttttggtgaccatttcactttccctacttaagggttgcactgtgtctcttagcgatcctgcagtcacacgtctcactttaggcaggtaacttttcctggtgtaagagatagttagttaccgctcatgcttactgtttagtgcttcagagctgacttaaaggttttacagttgtgctcaaattttctttgtggtatagaaaccttccttttaataacatagtagtaaacgttacatgtcatgggatggggttaaggggatgacagtagttacagatgttgggcctcagaacatttactgtagcttaggtggacttagactcagtgctcttctgccttgtcctcttatatactgggattgaaggcatgccagtacacctggctaaaattctaaataatttatactggttaaagctgacttcatgtagcaaaagttaagctacatagtcctttgaaaagttactttaagagtgaagactctttaggaactgaagaaactaaaactgaggaaagataggaaggggcagtttctggtgtcttccggtatttcattcagagtgtttatttagcatgccattacagcacctcgttagcactctcaggtttctcattgctatgctgaactgtgcaggggtaaggagtgggcagtagcctctttaaacatgataatgctgcaagttttagttgacctactagtcaagtgaggctgcctggctttactaggcctcactatatagacatggctgctccggctacttactgggtggccttggattcacaccattgtcctttctcagatgcctgggtgctgggattacagaaatgtgccaccacctccaactcagtttctttctattttaaaatatcttctgcattttcttttttttttccaaataggaaagtgataaagtaaattgactcttggtattttagacaaaataccacaataatctatgggtcataacctcaaataagacccaccaccaccccctcttggcactgagtaaaaatttagccagaattatccaagacactaaaatgtgaaatctccattgtcggcatgtagatttgtatgggaggtgacagcagtgccctgcagtgtctgcattgcccaagcgtgctgctgtccctgcacctctccatgaggccattgcttcataactaatttacacagagaaaatactctggctaatctgggacatttactttaattactgtatttaagttatgaagttcagtttagtagtaattaaaaatttaaggttagaacaatgggatattgtgggccagcaaggtaaaggtgcttgctgccaagcctgattacatatgatctgcatgttggaggaagactcctgacttctacaagctgtcttttgacctccctatggaagtcatggtgtggatgcccaaacatacacacacactctgaataaataaatgttaaaaacaaaaaacataatctagcctcttgcctttaggatcacgtccttcttacagtgcagacacagttctcttgagctccttctgtcttaccactgcttccgtgcctcagtccttttacagttttccctggaagataccctttcctcttacctgagagtttgttcattctttgggacccacattctagagaactgtgtctgcactgtaagaaggaagtgatcctaaaggcaagaagctaaattatttttttgtttttaacatttattatatatacactatatacatacatatatatattgtgtgtcttatatttcaaaatagacatcaatatttttcttctgtctttagatgttaaggaagaagacaaatccaaggacaatagtggtgagaaaacagaccccaaagggtgagcgatgcttgtgtctgtagacgtcatgcatgtcttgggggttgggcgggggcatgcatacttgagaaatactggcacagcctgagaacatacaatgagtgcaggtctgcgtgtgggtaacgtgcttaggctatttgaccggtaacttttatgtatgcaatcgtgtttttcatgatgctcatattactgtgcttagaatactttgatgttaggagacaggctagtttaaggaaaaaacattgggctaagtgaaagcctattggagaattttgttatcagaatgcatgtatggctaggtaggtgaagtgttgcggtaaatctccaacccaaatatgccctggcaatgaaacacaactcaattaatatgaatacatgctgtgtgcctagaatgggcagatctaccgctacactaccatcgtcaacaactgtgagagcccttagaacttgcagtttctccaggccacgtgcttctgctccacttttcttcttccccctcctctgcatcctcaccctcccctattttctcctctctctccccaccttcttctccaccttccctttatctgcccaatcatcagctctcctttattttacaaattaggtgggaagcaggtttatgggaagtaaacctgagtgctgactcattgcttgtttgtaggccctcactggagaaggaagtagcatcaaatataataagtaagccccagggctatccacacctgtgaagaggcttgagtctataatctcagtccactggagaggctgagcccagaggattgccatgaatttaaatccacactaagctacatagtgatttttaaatcgtcactctgggctgaagaatgagagtctgtgttagaagcaagcacgtgtgcttggtcacccagttgtttgctaattttgaaggataacttagaatgctcagttgtaaaacaactggaaagattaaggcttttattgctcagaaaaaaaagaaaaaacaacaacaacaacaacagtgaatagcttggtgtgaatctataagactgtgcaagtagccaggtcagtgctcctaaaccaagactattacttttaatatttcatgatcctctttcaacagagccaagtcagaacaactcagcaacccttgaatttgactctccaactttaggaacctcgaaaagtgagacgattctgggataagaaaccttccctgtttgaggacattggcttcattttatactgttttggcatggactgtatttattttcaaaatggcttgtttttgtttttcttggcaagttttattgtgagtttttctaattatgaagcaaatttttttttccaccatgctttatgtgattgtatttaaattgatgtgttattatgtcaaaagccggatctattaaagaaacaattggcctttctgagctgatttttccatcttttgtaattatctttattaaaaaattgtacttggatcgttttctgtctgtttattatgaaagcttgtttccaagtcaatgacttgatggtcttaagactggaacataccaaaaggaatgtcagtgtcagagaccatcactagatctacacagtgcttactggctctaacagctatttcttacttcaggaaaaataacagtgtcactttgtgtcaggaagactggtatttcataaattatttccaaattcataatctgtgcacttgggatgaagcaattatttttgaactgaatcaaatactcagaaattggaaattgtgaattgaaaaaagaaacctgtccaaccaataactggcccaacttaagacccatcccatgagccagcaccagtccctgacacttaatgatactctgttatacttgcagaccggagcctagtgtggctgtcctctgagaggctccacccagcagctgactaggacagatgcagatacccacagccaaacagtggatggagcttggggacttttatggaataggaggaaggatttcaggcctccaaggagataggaactctacaggaagaccaactgagtcaactagcctggacctttgtggctctcagagactgaaccactaaccaaagagcatacacgggctggacctaggtctacccacatagatgtagcagatgtgcagcttggtatttgtgtgagtcccagacaactggagaggaggctatgccaaaagcagttgcctgtctggatcttccagcttggctaccttgtctggcctcaatgggaaaggatgtgcctagcctcagacttgatgtgtcagggcatgtatacaaaagattgtaggcggtagaaacatacccttttgtgatgtcagtgtaaaatgaaccagctagcaaaacctggttcttgccctgtagtttgatcacaaccagggtttctactcaagaagtaaaacttaatatgctttggcattccgcattcattgagtgtctactcaccttttggtaataactgcagctgaatagaattttaagtgctttcacctaaacttagattgttattgtagaaagcccacaaatggagtttcctctcccagtgtgtacaacagaaaaatgttttaggaatttgccaaagttggtgaaaccatcctacttgctagaatcaggttgctgattttgagattaggagaacagagtggtagaaaggaggcagtcgtggagaatgttcttacattgtcctgaggttcgaaggtttcatgttcatgggttttccccttttgctatgtatttgccaggagtccatatcctttatgactaaggaagcaggatcccttcgctgcagagacttatttagtgtgtaattttcatgaaacaaagtgtatcctttggatacttcatctggtattgtgatctgccttctggcacatgtcgcataataattacagtgtgttaagttctggtctatgaatctactagaatgatgtcccagatgccacagtgattctccttggttggatctgggaggttctggtttcatagtttaaggctgtgaaactaagcagtttcagaacaggtgatttgggacggtaaaaatacacagataccagtaatttgtaaagtcaaactacagcccacattgtttggaagaacaaaggctccttttgcatagcagtaatcacaatttctacaaatgtgtaaaactgattctatgcataatgaataagatgagtgttaataatagcctgtcgggacaagggttgaaaatcaaaacaatgaggaaataacaagaaccaggtggagggaggcgaagttcatttttctgtcacaagagtgagatttataaatagaaaagacaacaaaacagaacctggtaaaagccatgggaatttttcaaaagtgaaggccaaagcttgacaccgaaccccacataagttacatacttaacagaagttctctagaccagcaaaaacaagtaaaataagatagggaaccccggcagtggtggcgcactcctttaatcccagcacttgggaggcagaggcaggtggattcctgagtctacagagtgagttccaggacagcctggactgcacagagaaaccctgtctcggaaaacccaaaaaaaaaaaaaaaaaaaaaagatatgggaaccataatggacttgttccacctcaaagaactttatttctttattttaaaaataaagggtctttgtttttggccaggggtagagagaaaattcagtggttaggagtatttgctgctcttacacagggtccaggtttgattccaaccacatgctcacagccatctgaggatctgacacccctttctttgagtaccaggtatacatgtataaaacacccatacatgttaactaaaaatagcttaaaaatttgacaagagttcaatgaaacccatgaatgtagtatcttgttaggaagacacaatgtccaaggtaattcttataaaggaaaacatttaattgaggctggctttccatcgcataggttcagtccattatcgtgatgggaagcatggcttgcagatagacatggtcttgatcagcagggaacaggaggagactgttccacactcggccgaacttgagcataggagacctcaaagcctgcccccacagtgacacacttcctctaacaaagccatacctacaacaagaccactaatagtgccactacctatgggccaaacatttaaacacatgtctaggggtgggggttagtacctattcaaaccactacattccaccttctgggccccatagtcttgtacctttaccataatgcaaaatgaatttaatttcaaaagtccacatagtttagcacagtttcagcagtgtttaagtccaaagtctcttctgagattcatgcactcttttttattagatattttcttcatttacatttcaaatgctattctgaaagtacccaataccctccccccccaactcccccccccccccgccctgctccccaacccactcacttctgcttcctggccctggcattaccctgtactggggcatataatctttgtaagaccaagggcctctcctcccaattatggccgactaggccatcctctgctacacattcatgtactctcttaactttaattccttataaaatcaaaaccaaatcacatgcttccaacatacagtggcacaggagatacattaccactacaaaacagggaggggaacatgatgcggaaataattaaccaaagcaagactgaaaaccagctgagtaaactccaaactctgtatctcgatgtctgacgtcaaaatgctctacagatctccaactcctttcagctttattaactgcaaggcgtttcttttcctggtctagttccgttccctgttagcagctttccttgacaggtatcctatggctcagatatctctagatttttggggtctacagggcaatccaggcttcaccttcacagcttcacagagtggcctctctgggcctccatgaagagacacccctgacacattcccagtctcaacagctttccttaattgtagagggaaattccaatatccttttcttctatccttgattctaatgccagaacccaatggtcaaatctgtcaagttctgctgctgctggtttgagctggaatatgcccccccccccccctgttcaattacaagttcaacagctttctgttttccatggtttctttcactgcctaagcttagctgacatggaacttggtctatagaccagctggccttgaactcagagatcacatgcctctgtctcctaaatgctgggattaaagggtgtgtacatgcctggacctaagcttttctttaattccattttacaaggtggaagattagctgggtgggatcttgccgcaagatcaccactcacttaattccactaaatatctttaatctgtttatctccttgaacaaagagttgggctttgttccacttcctggttcccctttaacccttgaaccatacgatttctatttttcctttctaagcttgatgaaaacactccttacaagagtggatcacaacacaaagtctaaattaggcttttctgagatttcttttgccagtgcaaggaaagtaatctaaatctcttgaacttagcctcaggcagactctttggacagagggcaaaaaacacccacatttgtcaccaaaatatcacaagtacaatctctaggcaacatactaaaattctcctctgaaacctcaatcttgagccaggccttcacagttcaaagaatgctcagagccatgttcttactactgtggcccattaagcagcttttaaagcattctactgctttcctaatacagagtccccaaatccacgtccctgcaaagggtcagtcttatcacagcagtaccctagtccctggtaccaacttcttagttagggtatcattgctatgaagagacaccatgaccaaagcaactcttataaaggaaaacatttaattggggctggcttacagtgtcagaggttcagtccattatcatcattatgtgtaggcagacatggtactggaggacctgagagttctacatcttgatctataggcagcagaatgagactttcacactagcatatcttgagtttaggagacctcaaaacctgcccccacagtgacacacttcctccaacaaggctatacttactccatcaaggccagacctcttaatagtgcagctcttcatgggctaagcattcaaacacaaatgtcttacacagagtcattacctacacaaaccaccacagatagattaaaataatctcttgtacttaattttacattgttaagattactaaaaggattcagcacttcatt

SEQ ID NO: 45 - DNA, 16 bp, synthetic construct
  source 1..16: mol_type = other DNA
  modified_base 5: mod_base = OTHER; note = uracil
accctcaagtctcctg

SEQ ID NO: 46 - DNA, 16 bp, synthetic construct
  source 1..16: mol_type = other DNA
accctcaagtctcctg

SEQ ID NO: 47 - DNA, 89000 bp, Mus musculus
  source 1..89000: mol_type = genomic DNA
attccttttcttcctttgttttttttgttttgttttgtttttgtttttaaagatcagggtctccctctgtcatcttcttgctcacagcttcggtgcgacccctgtaccacctcctgtcagctgctttccagagtgtacattcctgctgcctccttactgggcttattcaagcacatgtgtgcccagaagcattaaattattactgatttaataattattattattaattgttccacccgggcaattggcggcatggccaaaatcctatgttcaagagactgggggaagaggactgggagttgcaggcccgcctgggatacacagtaagggtcgtcctatgctagacagactgtgtctcaggtaaacacagccctcctaaaaggaaacaaatacagaacaaatgctgggacctggccatgaaaagaagctctgagttcttgatacactgggtgtggtggctcctgcctgtaagagttagaggcagaaggattgaagtttaagaccatccttggctacacagccttgagtcaaggctctgtgaaatgaggctctgtcaaaggtaaacaccaccatcttcccccttcccccaaatgtgcttccagaacctccagttttgcctgcaaagtagtgtattctcccatcttgcgttagggagtgaatctccacattgcacttggcagttaaaaggatgtaggatgtggtaaagctgaagtaacatggaaacagctgcaatactggcttctgccttacaatggcttgggtggtgggtggatggggcacctggagagatggtggcctagatccccaggaccctcataaaaagtcaagcatggtggtgagcgtgtgcagttccggctgctggctaaccttctcagctgaactgaccagctcttggctgagaaatcctgtttcaaaaactaagagaagtgacaaggcctaatgtcaagctgtgacctccgtatgcacatgcacagggcagcataccacagccacacaaatgagaaaaaatggaggctggaggagtggtttagagtgctgctgttggaggggactatagtttggttctcagcacccataatcagatgactgacaactgctcttaacttccacttcaaggaattccatgtcctcttctcatctcatgtgcctatatcacacccaacataaacacataattaaaataaaggggctggagtcgtggtggtttagcagtttagagcacttgttgctcctgtagaggaggtgggttcagttctgagcacccatatagcagcttgtaactgtacctccagttctgagaggatctgatgccctcttttggttttcttggtcctgcatgactctggtgaacaaaatattcctgcaagaccaaacaccatgcatgcaaaagaaaagtttggggttagagagacagctcagtggttaagaacagttgttgttcccagcacccacacatttaaaatctcatataaaaccatccagaactccagttctagggaatctgatccttcctgacttctgtgggcaccgggcacagatctggtgcgcataaaaataaaataaatctttaaaactttttaactgttattttatgtaaattaaaaaaattgtacatggattttttttttttttgcctgcatgtcggtatgtgcattatgtctgtgtctgtttctagtggaggccagacgagggtgtcagattcctggaactggaatcactgatggttgtgtgagttgtcatgcaggtgctgggaaccaaatttgagtcggctcccaaggcagcaagtactgagccattggtctaggagaaaaaaaaaattaaagtaaaaataaacctacagttgtagccatccgcaaaacacttgcttggcttaatggaactgtagacttgaccctctgtaacgtacagaaataagcacgagagggtgagaacgcctttatgaggcagaagtgtcgactcatctttcactggcactgggattcttccagagttgggatctgcgatgctttctgctttgttttgtttttttgtttttttgttttagtttttttgttttgttttgttttgttttgtttttcgagacagtgtttctctgtatagccctggctgtcatggaactcactctgtagaccaggctggccttgaactcagaaatctgcctgcctctgcctcccgagtactgggattaaaggcacgcgacaccacgcccgactgcgatgctttctaaaatatgggttttcactacagcctaagaattcaggtgtaacagccactgaaccttaccacggtgtgcttccctgagcactgtggccttttgcttaaaaagaatatcacaaagtgatagcaaatgccaggaagttggaagaacttttatgacacagttgtcagtgaggattgggaaggtagtagcgtgtatcctagcaggagggaggctgagacaggaggaacatcaagagtctgtagccagcatacaaaactatagaaactatagaaaaagattgcggctctctttcctcttactggttggagtagagaagtgatatttatatcagccaatgagaaatttttacggaattaactagtgtcaagtgacaatccagtgttgaagaatggatcccttacacccttggcaggttgacgtttctgcgaccaataagccaacccgaatcaacctagtgagctgaggcctcagtgtcttttccagagaccccgccttcccggtctgacaacccggtttgacttctattggctttcacttaagagtgacagttttattacccagtgaatttgtgagattagcccttcctcgtgccttttttattggctcacgttcttgagtggcagtgctttgaaccaattggatatagcagtagggttgcggactccgccccggaggccgggagggttgttcgctcgggtcgggtgtcgcctgagaaccggatgaggcggcgactctgaggccgagccgggagcgggcgtcgcggcgaaggggagaccgggcgggccggcagagcagagcaggaggaagcaacggccacagacacatcggaaccgagagtcttagtcgggggatccgcgcggcggaggcggacaccatgggcaaccgcgggatggaagagctcatcccgctggttaacaagctgcaggacgccttcagctccatcggtcagagctgccacctggacctgccgcagatcgccgtggtgggcggccagagcgccggcaaaagttcggtgctcgagaacttcgtgggccggtgagcgagcgcgccggcggcggttctggggctccggccgggagggcgggcggcggcctagggcgcggagggcgggcggggaatggcggaactgcggctcgcggacgccgcagcaccggtggcagtggataaggtctggggcaagagtctccatggctttccgcagggactggagatgcagatccaagtctgggactagtcgatgatagtgggcggtgtttggggcaagacggtcagtgccgacccctcttggtgcttctagttccggctatggctgggcaactctgacatcgctttacctgtagtccgggatagatccactcctctttccttctgctacctggaaatcttagtgtgtgtgctaagatgttgaagataacacttgccacttagccatccatggggaatgacccctggggtctgtctggccagttattttctggagcctgctctcttgcctggttagctggttatctgttctagacaggcttcctaccggcaccatttggttgtcccatctgtccaaactcactctgaagtccaccgttgtctggctagccatctaaggtgacctctcgtgtgcaaatgcccatctgtgccccttatttacgtgagttggcctctgccatctgactggctgaactttgggagcctcctgttatctggttctcttgggccgactggctctttgaatccatccattagtctgttctccattcccacccaggcatccctgcctgtccatcctctctgagtgacagaggttggagaacttgggaagaaacattctttaggattctttcttgggctaggatcccctaacctagagcttcccagttggaatgctctctagatagctctgggagcctcagcttgttccatctgtccagtgggcaaattccacttgcagctgtttgtaggatccaggaagtgagtgaaggagggatgttgatttactcctgtcccctccccctctgcctctccccgcagcagcacttctccattccaggaagtgttccatgtatgatgctgcagtgtgtgcctacccagtgttaacatttgctttgctgtggacattatcattgtgtgtgtgtgtgtgtgtgtgtgtgtgtgtccaagtgtgtgccagagtgcatttgtggaagtcagagggcataatctttgtgaagtccttcaaaatttatgtggggattaaacaccgggccgcagacttatatagcaagtcctagtatccattgagctatgtcattgtccttccatcttaaattgtagagacatggtctctcactgaccatgcagctcaccgatctcattgggtctctctctctctctctctctctctctctctctctctcacacacacacacacacacacacacacacacaccatgcagctaactgatctcactcagctattctcgctggccagtgagctccagggatccacctctctctgtctcaccctatcattgggtcacacacacacactcacacacacaccctctgttattgttgttaccagtcttttgtggaggcagaacatctctaaatttgaggctagctcactctacatagagtagagttctagggcagctagggctacatattgagacccagtctcaaaaaaacctcaaaagttgattcaagactgcagggactgggtacttggaagtgtacatttaggttgaattagctgcacctgtgatcttaggattttgggagtctgaggcaggagatttcttgagtttgagttcaacatgggctccattgttagtcctgtctccaaaaaaaaaaaaaaaagccatgagagggctgtgtggtgacgctcgggcatggagtacttgcctagcatgaataagacccaggggtcagttcccagcaccaaactaataagaaatattgttgatatttatgttctctttgattgcattcatactcaaaaacacctatggcttttaggtaatgaaataaaaattttatgtatatgtatatacacatatccctgtttgtttgtttgtttgttcgtttatttatctatttgttttttggtttttttgagacagtgtttctctgagtaacagagcctggctgtcctggagccacctttataggccaggctggcctctaactgagagacttgcctctacctctcaagtgatgagattaaaggcaagtccaccaagcctagctattgatcaatgttttaaaaagcatctcagaatgtagacaggctggccttgaacatacagggatgcacctagttctacctcctgagtgctgggatcaaaggcatacagcaccgttcctgggtgggggttattttggttttttgttttttaattttaaaaaaatttttgtttgtttattttgaaacagtctcatactgtagtccaggctgacctggaacttactatagcataggttggcctccagaattgtagcagcagttgtccgatttcattttctaagtgctggggtcacaagcacacaccactatggcattaagttcattttactctagaatgcataacgtacctgtgttgcaggactttgccaagaggcccactcaatcccattctttatttcctgctatggactttattaaacagagtcagaactatgagtctgtctgacccttgtgctgtttctttcctctgctgttcttgaatatccatccaggggcatctggttgtcagccaggagcaaaggagagtgtgccaagggcttgggccagcccagcacctagggacagctcaaaatgcttcctgtcagggctgccttagatttttctaagaacattagtgtacactaggacagctcagtgggcccagccccttgccgagacttgcatgcagattgctagccctggaggccaagggcttgaaatgtaacaggtttcacatgaggtccagatttctacagagcctgaaactaccctgaaggttttagtaataatgaggtagaggcctgaggccccaagcatgggtctaccaaagctgtgactttagtcaagggcattgattgggcattctaggccccaggttcctgagtgtgtgtctgtgcacaagtgtctgaatatgagtgtgtggggtcagagggcagccttaatcatggttcctcaggagccgtcgtctttgtgggtttgagtcacttgtagtctaggctgactggccagcgagccccagggatctctctgcctggaccattacagtgctgtgatgacaggcgtgcatagtgatgcctggcgttttacctgagcgccaatgattgaatcttggctgacattgaaatggctatagagctgaggctaacctagaactctggatctcctacctttaccttctgagttctggcattgcaggagtgcaccaccatgtttgggtttatgtagtactggggatggaactgagaactttgtgcatggtaggtgaggcagccagaccccaggtttctctgctctaagacagagctgtatgtaatctgggcagagtggcagtggtataatgtctgctcccccagaagtgaaaaagtaagactgagaccctcaccctggggtgagctgacttcacgggtaactcactggatcttctaaccaggactgcttttaccattttagaagcgttagcatttgaaaccagcatctgagccagccagctttgtgagctagctatggctctcaggagggcttggtttctacctaatcgttgctatttaattgagggactggcaaagaggctagctgggcttttttcccctaccccacccccatttggacaggaacccaggaagttggggttccagcctggaaagcggaggctagactccttcctgtgaatgtattgcctttctgaatcggaatctcagaaggaagtggggttacagtttgttccttgagtgtttgactgccttacagcgttacctggtgcatgtcactgaggccgagtcccacttgtctatgactttcacgaaggctggcaagatggctcagtagggaaaggggtttgccaccaagcctgatgacctgggttgatttctgaactccaatggtagaagcagagaagcaatttctgtgtgatgtatgcacacggagacacaagcccaaagaggatacataaatgaaatgttcgggaaaaaaatttttttaaagaaaaacaaaaagggagcccaatactgcagctcagttggcaagtgcttagcatgagtgggggttccacacccagcagcacagaaaccgggtgcttggaaagtggaggcagaagatcaggaattcaaggtgtgcttcgcagcagagctggttctgctagcctgggctacatgagatcctgcttccctgaaaaaataaaatagagttccagcctcaggcctagaacccaggactcagctaacctgggtgggctctgttgggaattaaataaataggtgtgtgtgtgtgtatgtgtgtgtgtgtgagtgtgtgtgagagagtgtgtgtgtgtgagtgtgtgtgtgtgagtgtgtgtgtgtaaggggggggggagaaaacgtgtaatagcaataactcttccataggccaagtgagtgttcactggacagtgttttccagtgcttagcacaactttgcctcatgcctagggttatggccttgagccaaataaccttccagcagagactagatgaagacactggtgccgtccaatagcgctgggaaacggagaggggaagaggcagggagaagttgggaaagataccctgaagattggagaagcatcccaggatgtggaaatagccagtacaggttctgagctgacagcgcacctgagagtgtcaggaagctgaactgtggagtgaggtttaaaagggatggctgtggggcttggcacaggccacttctctcctggggaggcacattccctgcctctggctggacgcttgggtgggctgttccacatggtcagttttctctttacgcatttcgcctgttcagaccttctcccattttccttggatcaggaggcacgggtggcagtgtgcacaggtacatttggacatgtgttcatatgtgtgtgggtatcagaggttgtgtgtggtaggtacagtgcatgtggtggtcagaggacagcttgtaggagttgttcctctcctttgtgggtcccatagagccaactcaagtcatcaggcttggcagcaggtgtctttatccagtgagcatctcaccaggatagtggtggtggtggtggttgttgttttgtgttttgaaacagggtttctctatgtaaccctggctgttctggaactctgtagatcagggtggccttgaactcatagagaccagtctgcctctgcctcctgagtgctaggattaaaggtttaatctgccatcactacctggcttttcttctgtttttaatacacaatgttttttatacgtaccccaggctggtcccaagcttgtgatcctcccccactttcccagtgctgcaatggcagccggttctaccaccctggactcgattgagcatttccagtgctgggtaagagctggccaccgaagggacgcccgttgctctcactcttgcagttgcttggagctgcagctggtctaggctgtgccaggtttgaatacaaatgctgaaatgacaggctccttggttgctgggagaaacttgggcatctgctgaaccctaatttacaggggaggggccaagtgctagctgtaattaggcccttattaaagtgtcaatgtttttggagaaaatacagtcagtaatgagctgctcacatggcctctctgcaagctgagcgaatggaggacgttgtcatgcctcctgagccagatgctacactaaggaagtctgcttccatgtgtggtagaggtttctgggaggccagccttaactttagccagaggctaggagggcactttgcttccgtgtagtttctctttaaggtctacttcctctctctctctctctctctgtgcatgcttccatgagtttatgtgcaccacacatgtgcagtgcccttgaaggctggaactggagctgcagacagttgtgagctgccatgtggtcctctgcaagagcagtaagtgctcatgactgccaagtgtcttcctcacacctcctgtttcttttgagatggggcctcatgaagcccaggctagcctagaatttcctaagtagctgagggtgaccttaaactcccagtctttcctgtgtctgtgtgctggaattccagatgtgtaccatcattctggtttattccttgctgggatggaacccagggcttgttgcctgctaggcaactgaaccagtctaccagctgcatcccacccaagtttttgggagatagttgctgcttttatacttttctcagttacagttacaggttgggaaacagactggagagggaggtgacatttgtcatgaaagccagatttctttaaggttgtcaggtgtgcgtcttcactgtcaagccttagttgggcctaatggaccaggtgctggagacttgacagggacccagaactaactagacagaaaaatgggtgggcaggtaactaaacaacattctggaagaatgagaaatctggtgatagatagcatggagtcagaagtgagactggttttagcatccactgtcacatggctgcccctcagttgaactggagactggtgggacagaacagtatccacacagaaggaccaacaggtagagtgccctgcacatacctggttagctgatggcccgatgcagatttggcatagcctgggatacatggggctcaggacagtacctgtcagtttttaattacaagagcagagtgggcacagtagcagacccaggccgtccagcttcccggcccctctggagtcccgctctcagcggagggtgcctgctgttcttaggccaccccttgtcttccccacagagcaccctgatcactctgcttagattgctggacagctgctttccctagctgagtgtgggagctcacgcctgtaagcacagcgtttgagaggctgaagcattagccatagttcaaggtcagtttggggcacagagtcagaccttgcctcaggaaacaaacaagcaaacaaacaagcaccagaacaataacacgaagaaaagctggctgtaatcccagcacctgagaggtgcaggaggagagagaagtttaaggtgtgcctcttctactcagacaagtggaggctagcctgagctacagaagacctgtctcaaaaatcccaaacaaactgaagggaaaaatggtataacaacaaagtccacaaaaacaatcttcctttttccttttctttttttttttcagatgtatatggtatgcatgtatgtatatgtgtgttttcatgtgtgagcatgagtgcccaaggaggcctggtatctcccactgcatccattgagtcagggcctctcaatcaaactctgagctcactgatccagctagtctggctagctcattgatccattgcctttctaggttggactcagaagcgggctacaaaatccgcctagcatatatgtgggttctaggaatccagactctggatttcttgtgtagcacttaaccgctgagcccacagctggtttgtggtttgtttgtttgtttgttggtggtgtttttttttttttttttccttttttggatttttgagacagggtttctctgtatagccctggctgtcctggaactcactttgtagaccaggctggccttgaactcagaaatccgcctgcctctgcctcccaagtgctgggactaaaggcgtgtgccaccaccgccaggcttgttttttttgttttctgttttttttttaatgtatttttaatgtacactggtgatttgactgcttgcaggtctgtgtgagacagtgttggattccctggagcaggagtttttggaaagttgtcagctgccatatgggtactaggaattgaacctggatcctctggaagagtagtagccagtgctcttaacctctgagccatttcaccagccctctacaactagctttctggcctgggcacttctagaaatctttccacgtcagtttccagttgtagcttggagcatcctgaccgctgagtgaggcgggcaggagcagcagttggggctgaggtagtcgtgctgttgagagcagcagctctctcggtgggctgttagctacagccttgatgatgtgcttaccacaccaggaggcctgtgtgctccctctcactagcaaaaatcccttggagtgggtattatgaagccaacctcttggcttctctcctgggccttacccagaggcttgtccctgtagattcccctaactgagatttcccagtcttcctcctgagcctggaataaagtctttggcaaacttcccaggtgccaaggtccggagttctgtgtgtggaggctttgggtgggcttcacttaaggctctccaggtgcctgtgtgtaggaggctggagcggcaggcatgcagaacatcttggaggaggcagctgtcttcttgctgaggtgaagcagtggaatgacttgctgacctgtgctttgtgcctgtgtggctgtgaagtgcgctgtggcttatgtgtggaccttttggtgacagacatcaaagctcttttgccctcattgttccaggactctaaatgcacttcatggaaggagccttgtagagtgtagtgtgaatggggaaggctacgtgccctccccctggcggtggcagccacttcacagttggagttggatacatgccttcggactctcttagaggcctccctcctgtccctatgtcccatgtgccacctatctgctcagagccactcagcttccacttgtagaacaaaaccagcgactcccaagaacctgccgatcctggcaccacactgcttgcgcctcctcctcttcctccccgtgggttcttagccacctcgctctcaccatatcccctctctcaagactgtgccttatgtaatgtatggcactgtggtcttgagttccacatgtaactgggagtgaccttgaactctgcatctggggctggagagatggctcagcagttaagagctctgactgctcttccgaaggtcctgagttcaaatcccagcaaccacatggtggctcacaaccattcgtaatgagatctgactccttctggagtgtctaaagacaactacagtgtacttacatataataaataaataaatcttaaaaaaaaagaactctgcatcttcatatgtctatctgagtgctgggattacaagcactcaatacctgctttgtgtgcagctaggcatggaactcaggcttgatgcacactaggcaaaccctcaactggcatatggccagcctgtttccagtcctgttattatcattgctatattggtattcgtgggtgtatctatatgtgcgcctgtgtggaggggagtgatggaggccaaggactattttcacttaaaaacttttttttttaaatgtgtatgagtatttctttctgtttcatgtacatgtcacccaaggaagaggacattggatcccctggagatggagttacacatggttgtgatccatcttgtggttactgggaattgaaccctgtttctctgcaagagcagcctgtgctcttagctactgagccatctctccagacccttaattataattttttgagctagcgtctcactcagagcctggtctcattgtctgtccttccccttctctggagttaggatcacaggcacatatgccgtgctcaggcttttctttttttttttttttttttttttttaagatttatttatttattatatgtaagtacactgtagctgtcttcagacactccagaagagggcgtcagatcttgttacagatggttgtgagccaccatgtggttgctgggatttgaactccggaccttcggaagagcagtcgggtgctcttacccactgagccatctcaccaggcttttctgttttaggaagtgaactcatgtttggccagcaagtgctttatccatggtgatgatctccctagcctatgatttgtttgtttattttatttttgatacagagttttgctatacagctcagctggattcaaacctgaaaagtccttgccttagcctccctaatgctggggttataggcagtgtcactgtaccttggtcctctgctgcctttctctagaaaagctgaggtgaggtctgactgtgatccacgggtatgtaagcaaggccagcaaggctacctgggaatatccctactccctggtgaagaaggaggcacccgagagcctgctcctaacgcatgttcccagactctgatggcttggagagcaggcaggcattgtcctactgtgatgctgcttaccgaggatacaccccagcacacagaggccattcccatcgaaagccccagaggtggcctcagtacacaactctgtcctggctcagggcctctgtcttttgtactgatagatgaggtttttctagagttgttgttctcaatcttctttagatgacccaaccataaaatgacttttgttgttacttcaaaactgtaatttttgttactgttaagaattgtagtataaatatctgatatgcaggctatctggtatgtgacccctgtgaaagggttgtttgacctccaaagtgttgtgactcacagattgagaactgctgttctagaggatggtagctttctggtctggaatgcctgccccagcatccttagctgcagggaaccagccagtggtgagagtgaccttgaattcctgagccttctgcatccactattagtgctggcattcagagtgtgccactgtactgagaggacatcatggttgctagaatcctcatggctacccagtgaaggctggtagagatcaagtccaggagcctgtggccggtatctggagccctgctcagctcagtcttcctgtgctttaggtccccttggccatcattttgcaatgttgacttcccactcagccaagcggaaggaagcctggcttctgtagcctttgcctaggtggcactgccaggggacactgtcttgcataggctgtcaaggatttgggtataagttgttttctagggaagggcctacatggggtttgccctggcatgtggtgaaaactgagtcttaaacttttcttttctttgaagttttcttgaggtctcatgtcacattgctaacctcaaactaagttaaatgcacagccagggcggggcttgtgcttctgtacttcctgctgtcgcctccctgtgccgacgttactacaccaggcctagatgctgtttttttccctccgacaacctgcttcaacctcaagtactgggattgtaggcctttccaccacagtcatgcctggatttttaaaaagttggtagttttatgtaatctaggctgggcctcaaacttctgtcacttctgggctcctgatcttcctgctctgctgcctgagtgctgggattgtcagcagatgccatcacatcaggctttaaggtttctcatggcactggggatcgctcccagggctctaccactgagttacctcttcttctttaagaccacattagagttattaatgtaattattttgggtattagagatggtgttctcctgtgtagccctggctgtctatagaccaggctggctccggattcagagatctgcctgcctctgtctcctgagtgctgggagtaaggtaaaggtgtgcactggctacagcagagttataaagagaggggctgggctcaatgggagaaaggtgcttggtgccaagcctaatgacctgaggtgcatccctgagacctgtatagtggaagagagcagactcctgaaagttgtcctcccatcaggaccatagcaattccacctccaagcagagaaacatcaataagatgcaatttaaaaacatttagggcatgttgcccatgtctgccttccttggcctctccctactgcaggctctgttaagactgtcactgttgatggaaggcccctgtcacatggctgccgttgccctcctaacacaggatcgcatgtcccctgcttccctcctctgctctaggctctcaggctgtccgtcctaggctctcatgaccttggactttgatagaacacaaacaacagtaacttctcccagcttatctggcttgttctcacagctgactgtgagttttttcattttgggtaaggaactactttaggtccgctttgtgcatcttgtaggactccataatgtgggccagcaggacattcccagggagagggcatctgccatgtttccttacttgaaagttactgggtttggttttgcatatcttatcctttgtaagtatccaggccctaaaggttttatgcatgaggacgcaagtgtatgtgtgagtttgtgtgcatgtgtgggttcttgcgtgaaggacaaagattggtgtccaatgtctcccttaagtactgtggaagaatctctcacttacacaaagcttgtctaaatctgctaatctggctagccagcatgttctggggatctcctgtctctgcccacaaacactggcattacaggcagataccacaagagcttggcgctacgttagtcctcaagtttgcatgtaagtgctttgcctgctgagccgctaagccgactttccaaccttcattttgatttttctaggtctggggatgcaactcatggcctcatgtatactaggaaaggcctctacccctgaaccagacccctattctctctccatgggattctagtcaggggttctaccactgagccacgccctcagtgcctcactgagggattctaggcaggagttccacctttaaataatacccctagtttctcactgatggatttcaggcaagtgccctcctgctgagtcggctttccagcccctaattagggggtagaggggcagagccacacctccaacccctcattggggcattctagataagcactctactgttgaagcccgtttcctagctgtctcttgcattttatatagattataaagtgtaagttgaaaagttgaagagttgttttatatttttcttttgcaaagcattgtacaatcttgtatttcattggatggcttttgtcagatgggcacagtctcttttggatgctgctggagcactgcccagcagttcccacatctgcacaagccacatctgtttatgagtgcagctctctgcttagtgttgaaatcaggcgccttgaggacgctttgttctttttctcaattgttttgaatgttctaagtcctttaaacattaattttgtttctatttgtttgtctttttgagaattctatttttgtttgtttggtttggttttggttttggggggttttgtttgtttgtttgtttttgttttttgagacagggtttctctgtgtagctctggctgtcctggagttcgctctgtagaacaggctggtctcgaactcagaaatccgcctgtctctgcctcctgagtgctgggattaaaggtgtgcgccaccacgcccggcaagaattcttactttgtttctcacactgacctggagcttgagccccttctgccttggcttgtgcgtgctggtattagaggtgcagcaccatgcccagctctgaatgcccattttacagttagcttgtggatcgcttgctctgtctgtctgtcaggctatccatctatcacccatcttagagtttcactaagcaattctgcttgtcttggaactcactgtgtacacaaggctggctttgaacatcagcctccaagtactgggcttaaagatgtgggtccccaaccccagttacgttgtggatttctactaaacaaaataaaaagctaagacacgatagctcagggatggagacctagtcaactgtagaccatgtttctatctatagcaccaaaaaaggaaataaaaaaggctgtttataaagacccgtcgaagcctatcctgtcctaggctggcttcagaatcatcatgtatctgagggtgaccttggactcccggtgctcttgactttacctcccaggtgttagcatgacaagcatgcaaccagcacttaagtctgtgtgatgctgaggacagagctcaggactgtgtatgccaggcaagcacttgaccaagtgagccttaggcccagggagggtagggttttacggtattcatttttctgcttcatgtgcactgtttgtagatgtaggtatcagaggatatagttgcaggtgtgggtaaggtgtaggttctggccgtcaccttgttggagatgttcagtgctgtgaatgacagacatatcaggtggccccagtggggaattctgtctgtgcacaccaaactcctattagttttgattgctgtgctatttcatcagctgtccacgcccctgcattgcctgctgcctcactcttgttcatttgactccccctccctgctccccatagtaggagcctgtccaaaacccaggagcacatggtggtgcaggcctgtgctccagccctgatgctggaaatgttggaaacgtgttaagtatattggttagaaaatagccactcagaaaacatcagtaattgtgtaccacaaatgcaattgtatattttttcttttttatttttctatgtagacggggctgttctcagactcattacagaggtccacatgcctctgccttccaacatgtgagactaaaaggtgtgtggcatcacatccagccttaacaacgtgaaatgcgcatgagggggagtgtgtgtgtgagtgtgtgtaagtgtgtgagagtgtgtgtgtgtgtaagagtgtgtgaaagagtgtgtgtgtgtgtgtgagtgggtatgtatgagagtgtgagtgtgtgtttgtgtgagtgtgtatatgagtgtgtgtgttggtgatgagaggacaacttctaagacttgcttctctccttcctctctctgtcttcaagatttgacagacagcacctgctgctgagccatcatagcccccttctttattctattccagccccaccaagccttgataatcatccccctgatcagcctgaacctgtctgcacttcctgaccaccgttgtccctcaggccgctctgtcacttcctgcctccttcaggcttccagggaaatgtctcttccccagggcaatcccaagaccctatgctactgtaccattggcccccttgtaatttcttttagaaacatttatactccctctttgtttgacctaaggtttcttcctcttcctgggtgtgttcctgctttgcaggtacctgggccatctccctgtgtcaccttacacccacctttgctttagtcaccaagagtcactcagcaaaggagaaaggtgtgccctttgccacactgtgacctgggccccacccagctggaccaaaacaatttgtttagtgctctgtggagctggatctcattgagacactgcacctacatacagcaaaaggaggggacaaggcctgcctgggctttgtgtggacagcaattggtttttttgagacagggtcccatgttgcccagacaggcctcaaacctagaagaggctggccttaaacttctgatctttctgcctttactttcccagtgctgggatgacctagcaccaaccacatctgctttatgggggctgaagatggaacctggggctcctatgtactaggcaagcccagtgccaaccatagtcgtagagtttatgccgagacagcaagtcatgagtgtgtgtgaggcacgagaaacaagccaatacaagtgtagaggcatgaagactagtgtggggaatggaatcaatgtctaatactcatatatgaaaacacaatgtcatgtacgtgaaggcctttttaaaattgtaaaagcaagtaagcaacaacaacaagccaccaaaaaccaatagaaagacagacaggagaagccagccacagaagccacacgaatctacttggaagccacacgaatccacaggctagctccccgagacagagtggagttgtgcttccaggaggtggcaagggagggagtgagtgagcgcgtgggggacgtagagcttggggagattcaaatgtcctgaaatcattgagaatggtttcacaatctgaatccacaaaacccctagcagccacttaaaactggtggatctaaaagcctgtgacgcactttcctgaaagacagtgaagccagaggcagaaagcaactctcccatccccctgccttacgcctacaaaagaatgaatgccttgaagacaaaactcagtgctgaggcagggtcactggagacaggagcattgccaagagttcaacctgggctaaggtgtgagagaccctctctcaaaaacagtaggagccatagataagagaagcatagattgttcttctgagatggacttagccacttcatggggagctccagccggctccagccttgcgagtgccttaggtcctgtgcgcgcacgcacgcacgcacgcacgccagccactccttcccctgagtttgggcacctcggctcatcccacaaccaagctcttgtggtactgtgtctgtgtgtgtggtttggactcccaggagctgcctgtttggcttcctctcctaatggtttcaggagtccactgtctcctgtgctgtgccaagctccgggcatttaccaattttacttacttatttttggggattggagattgacctgctcgcatgttcgacaagccactctggcactgactccatcctaagccctcctgtgttttgggatggaatctcatcatgtagtctaatccagcctggcctgggtcagcgagtgtactgagaatagtaaaagatgccttttagacaggtagaaatagctcagtcaggatcccactacacccaaggctgcacctgatagccagaacccagtgacatcactcactccgaataaccacagtgctagaaccccatgatacccactgtacagtatataagcgaccaagagcagtgctctgctctgcacatctggagagatgacaccttgatccgtcatgtggctggagagtctccccaaggatcctgacccttgaatcagatttccattagacattagacctgaccctcagctaaatatgatatgtcgcacaggtgagttaccttagggagataggcttagaaagaacataggaatttagaaccttgtgtgatgaccatcagcaaaataaaatataacttttgttatattaactacacatactccaccctcttgctcaaaacactttggggtgggctttaaggtttcaaaagctcacaccattcccagttagttctcattctctgcctcttgcttgcagatcagatgtaagttctcagcctacaccagatgtactactccagtgctatggctgcctgtctgctgcagtgttccccaccatgctggtcatagcctaactaccttacctctctggtactgtgagtccagctcaaatgcttccttttacaagttgcctttctttttggtttttcgagacagggtttctctgtgtagccctggctgtcctggaactcactctgtagaccaggctggcctcgaactcagaaatctgcctgcctctgcctcctgagtgctgggactaaaggcatgcgccaccactgcccggcttataaattgcttttctcatggtgttctgtcatggcaatagacaagtagggaagagttgggtgtgtggtacatgactttaatacgaccactccagaggtagaggctggcagatctttatgagttcaaggccagcctgatctacataatgagttacaggatagccaaagtatgaagaaagacctgtttcagaaaaaaaaaaaaaaagaagaagacattagctgaggcatctttttcccctcccctctgcccctcctctcctcccactgatttagttatggttgcttgcaaaaggtgtgtatgtgtgcctgtatgtgtgtacctgtacgtgtgtgtgtgtgtgtgtgtgtgtgtgtgtgtgtcttactgctcatgacactgcctctcagagccctttcactgcctgtagcccctgggtgaggggatgaggctcatggccccttcctctgagcataatgaaatgctggcaggctggatcctgcacagggcgcagggctgcatgtagctgcaggtgtgccctgtccagaagacagtttctcagcattcctccttgtcattattttatttgctatttatttatttgtttgtttgtttaagtttttcaaggcagggattcttttgtacccttacctgtcctggaacttgctctgtagatcagattaaaggcatgcaccacaacacatggcgttgttgttgttgttttttgttttgttttgtttttttatatacagtctctctatatagatttggctgttctagaactccctgtataggcctcacagagatctacctgcctcttgagtgctgggattagaggcgtatgcctggcttcttagtatctttcttttttgtgttttggtttttggtttttggtttttttttttttttttttttttttggtttttcaagacagggtttctctgtatagccctggctgtcctggaactcactctgtagaccaggctggccgtgaactcagaaatctgcctgcctctgcctcccgagtgctgggattaaagacgtgcaccactacgcctggccctcttaacagctttctgttcctgattccaaatgtttcttgaaacattggaagcattgatacagctgtcctgttcagatctgtcccgagcaccctgcggtcactcttccatattgatcatgtccacatgaagtgttgctcactgcagaggcttttctgagagagattgagagcagcctgaatcaatagttataaactttagcacttagaacagtttgtcagttaacaggggtaggttcagctgggcatggcggagcatgcctttaatccccacactcaggagacagacacgagatctctgagttcatggccaccctgattacaattaccgttccagtactacatagagagaccctatttaaacaaagaaacatgctgggctatggtggtgcacatctttaatcccagcacttgggaggcagaggcaggcggatttctgagttcgaggccagcctggtctacaaagtgagttcagcacagccatggcaacacagagaaaccctgtcttgaaaaaacaaaaacaaaccaaaaacaatcaaacaaacaaacaaacagaaaccaaagaaacaaacaaccaacccctccccccaaaacaaacaaacagacaaaaaacctgtagtcaattctcccgcaaatcctggagctctctaaccaccagcttttggccagctttacagaaccagcattgagtttcttcctgtagagtggatgtcctgtggttagtttaccccagtaataatcctgatatcattgctgtgccagtggacatctctttccagacaggttgttctggtggcctgtgggccacagccagaaggaccactgatggctttcttttctcagaggcaacagctgtccattagagtgtggtgtcttcagaggtggggctaaccctttagctctgctgggtgaccaagtgcagcagccatagcctgtgatatgccccgggcctccctgtctcctctggcacaaaactcccttttacgctcacagcttccaggaggaagggaactgtttgatgcttccgtctgacttcctgagtcgggtgaagaatgctggccttgcagggtgagctcagaagtgtgccctccagtccattctgtgggagagtctctgaagagctgatgctccttctcttcagggtctcttacaatctaccatgaagttgctgcctctgggctttttggagggaagcttttttagtaattttttttgtttgtttgttttttcgagacagggtttctctctgtagccttggctgtcctggaactcactttgtagaccagactggccttgaactcagaaatctgcctgcctctgcctccggagtgctaagattaaagacagcgccaccaccgccctctgcagggaagcttttgaacacttaattcaggccctttattccctttacgcctccagagacttcaccatgttcagccttttggtttctttgtaatttttttcctccagggttttcaatttcgcccatgctgtccttttagtctttttcagataatattttatgagactgaagagatggttaatggcacttgttctttaagaggacctggttctcttcccaccacccagttggcagctcacagtcctctgtaactctagttcctggagatctgacaccctcatgtggtctctgagggcactgcatacatatggtgcacattcatacatgcaggcaaactctcttgcacataaaataaaaatacaacagatcttagctgggtgtgatggagtcagagacaggtagatctctgtgagttcaagatgaacctggtctacatagtggctccagactaggtaggctacagagtgaggccttgtctcaaaaatatacaaatgtttacaaatattagttcattcattcatgatttgtgtgtgtgctagtgtgtgagtgcgtgtgtgcgtatgtgtgtgtgagtgtgctatgtgtaggagtgtatgtacatggaggacagagggcagctttagagttagctctccatctttatattgcttctggatcacactcaggtttctgacttgcacagcaagcttgcttacctactgagctgtcttgacagcccctcttcctttttttttttccatttggttgtgatattactacatttattccaaatcttagcaatctgagtcttttttctttgggaaattttggatctttcctctcctctcagcgccccaagcccccccgcccccactttttaaaaaacagaaccaagttgacctccaattcaatgtctatgcaaagatgaccttgaactcctgcctcccctacctctactcccctgtgcagtgctgggattgcaggtgtgcactgccctgcctggcttaggtggggcgagggataggacccagggcttcctgtatcctaggggggcattttacctttggaaccccaggccttgaggctttggtctgtttgcttttaattcttctgtagccatcaggcatagcactgctgacgctggccacatttattggcatttcctcaattccctctgtcttagttcttccttagctttctgctgaggctagggaacacatttccctcccctcgagcccgccgctctctgccacagctgagcacgcagtctcttttggagaatgttctctgtgcctttgagggttgggaagatgtccgagtgggcaaggactagtttggtgtgtgaaaggccctgaattctagcccatcttgaagggaaaggaggaaaagtagacaaagcccagagagtgggctgctgcttctggatttgggcacaaggctttctttctgccttgttgctttgggtggtattcactctgaatactcttgatgtctactcatccgtcattccagtttctttcctctttattgccagggagtgggggatagacagggtctcactgcttccctctggctggcctggaactcacagagatctacctgtctatgttttttgagtgctatgataaaggtgtgtgccaacctcatttgtttctttaaaggaaaaaaaaatttgttaagctgggcagtggtggcacttgcctttaatcacagcactcgggaggcagaggcaggcggatctatgagtttgaggacaacctcgtctatgaagagagttccaggacagccagggctacacagagaatccctgtcttgaaccccactccctccaaaacaggattttaaaattttgtgtatatgagtttttgcctgcatatgtgtatgtatagcacatgcatgtccgtgtctgaggagaccaatagagtgtgctgggtgtccttggaactagagttacaggtggtatgggtgctgtgaatcaaacccggctcctcttaactgctgagctagctctccagcctaatcccacccaccttttgtgtgtgtgtgtgtgggggggggtagtagtcaggctgtctctctatataacctaggctaatctggacttaaactatttttgccatattcctctggaatacaggcatcctaggtgtgtgttactcttcctggccatttgtcaattactgaaaagattgtcattgtccatgcaagccagaggagcagactttgggccttcagagcttgcatgaatatctgcgtaggtgtagttgcctgctttcattaggagggacacgcaggtaggaagtccctgaagacgctgactggctagatgagcagacctacaatctctgtattcaggagagacagacacctcctcagtaaatacggtggagggcagtggaggaagacaccgacctcagcctgagtccgtgtgctcaggtacacacgtgcactctcacatcacccacacacaagcaaaagacatccacaccgattgcattacctaattccctggccagttctttgacatatcacatggcattccttgactttgcctcacccactatctctcatcactgtcttacctggacttctggttgttcttggttgtcattctcctgacagtgcctgagggtcaccagacttgtgtgagctgaaggacctgaaggaagcaggtgacagttgaggaggaagcaggtttaaggtggtgttgagtcacctgacaggaaaggctgtgctgatgtcctcacaggggtgccaaggagaagggcatgtgcttcttgtggttcttggagttgacatccccccccagcccccacaccattcattgcctttgtgacagcaccctgcacatggccgacaccacacctcgattcctgtgccacctctgcctgcttctactggtgtcctaaggacatggtcacatgttaggaccccatctgcaggtggagacactgtgacagtgatgtccttgtgcaggtcacacatggagagcctgtgactgaaacatcaggctttagactctacccgtggccacccatattcttaattggtgtcaggaggaagcggcaatggcctcctgtgttcagagttcactggcgtcttcttaccctggctcccagatgtgctggggatcagcagacagtgctcaaaagctgagggcagggttgcagcagtttagctgggggtctcagtgctcggggtttttacctgtggctttggtctgagttactttaacctgaagcttcagattccttatctaccagatgtgagtagagaaagccgttatgtgccgttctagtacagaataccaagtcagggggattcagggttcagatcagctggagctacattttgagactttagacaaccaaaagacaaaatggggacaggataagcctcttgggagtacagcaagagtcccttgttcatagtcaggccagctcctgtggggtgtgtgtaccctatacaggccatttattagcaggtgtttgtttgttttttgttttggaatgggttcatgtagcctaggttggtctcaaactcagtttgtagctaaggatgacgactactgatcttcttgtctccatctgagtgctgggatgaaatgcgtgtgctgccatacttattttatgaagttctggtgatggaactcaggcaaacacattgcccgctgagtgccagacagtactgggacatggttccctgtccttgtggaactgacaggtaggttacttcagaactgggaggcaagaaggacgtgtgattgctactggaggacaaaaatccagagacaagggggggggggagcagggcagctgtcagaggagcgtctgcaaaggggtcagcctgtgaggtgaatggctatggaatgttctggtggtcctccctagggaacagttgaacccatagagttgggaatccacatccaccagccatctaacacagaagctgccacagaaatggcgatcgctctcagttcctcccttacgctgccttttacacagcacagcacctggcccacgatacaaaagtcttctgccaaggacacagggcagggctagacatggacacagctcttccacgagcaggtctgagacatcaagggccctgaaagggcggagttgagagccatggcgccacaggtcagacagcttccctgtcattctgtaaaatgtttttagctattcgatacaactggatgatgacaggtcagatatttgcctgttataaagttataaattgatctgcaatcacctggaattcgcatcaccttggagactgcaacataacttttgttttgtttgagatagagtctcatacagcccaggctggtctttaactaacagtaatcctgcctcctgagtgcaagaaatttcagatatatacaaccactctgttttctcatatttttttaaggcagggttttgtggagcccagctggcctcaggcttactgtgtaactgaggataaccttgaactccagatctaccggccaccagtgagtggtggcatcacatgagggccaccacacccagggaagcaggtgctgtgttatcctgagtccatgtgccagctgtctccctgaagaaaggatgcaagcagatgacctcccccgaggctggcttagtggctatcatgcttacctggcatgcacaaatctctgggctccatctgccagccagacacagtgctgcacacctgtgatcttagcacttgcagaggcaggagatcaggagtgcaggctcttactcagcttcagagaggcttcaaagcctaggctgcatgagatcctacctcaagataaagaaagcaaaccatcaaacacaaaaacatcccaagtatggagaagacttcctccatacagagcgtactctatgctccacatactggagggtgccaggccctggactatttctcagaccggttgccttagcctctcctgctgggtggacatataggtcattagtggctttctcttggacatcttgctatctgtcatggttacctcatgttagcagtgactggtctttgactgtggtgtctatgtcctggatcagtgtgctggggagcagatctctgaggagaggggcctcatctcctgtctattctggaggtgttcatgttctgctgcagctgatcatctgacaaagtgaggaaaaagaaatctggcagggcccggtgggtgttggctcaccaggaagcagctagacctctctgaggaagggacaacctagctgagcccttagtgtacacaggaaactagagctgtcataagaaaggtcactgtgtgaggataaacagaggccctgaggcaggaagatgctgtgctcttcctcctctcaacccagctccagggaatcgatgggggagtagtggctgatcgttgagtaataccaaggcctccaaggcccttggggggcaggtggaaccccctgaaagttacaagcagaggagtggttacaggttgctccaagtttactttggttgtataggatggatgctgtcatgggtgtgggtgtgcctgccagaaatggatgatgggtttcttttgctgtattgtctaccataggtttttagactggggactttactgagtctggatggagttcatcactttgggctcggctaatgagtcccataaaccccaggggtctttctccatgccccaagtgctgggaggtgggcatgactgcttgactttgtgtgggtactggggatcctcacctgctccttcctcacgattgtttggtggacacttagtgaccaagccatctccctagccccaagaatgatgtatttttagatagtatggcaaaaacttggtaatcctaacttccctttggggcaggtgtcattgtgcctgcttaaccaacaagaaagctgcgggagggctggtgagatggctcagtgggtaagagcacccgactgctcttccgaaggtctggagttcaaatcccagcaaccacatggtggctcacaaccatccataatgagatctgactccctcttctggagtgtctgaagacagcgacagtgtacttacatataatcaataaataaatctttaaaaaagaaagaaagaaagaaagaaagaaagaaagaaagaaagaaagaaagaaagaaagaaagaaagaaagaaagcaagcaagctgcgggagctagaggtggcccaactgaccagattgtccaggaacgttctagaagtaatccagttctggattacatctgggccttgaggagttctggagtcacagccaccaagacactctagctatttcctgtccttgttccccgcccaggctggctctcggatgtgactgtgagcaggtggcactaggcctgagtgccaagggtgggactgatttgggtcctgccggattcctggctagggagggactgctgggcagaagctgggtggtacgtggtaagcagcgccagaactgaactcacatggccaggcaggtgccgggttgccactaccaccacctgagcttcagtgtccttgtctgtgaaatggcagagtcagtccttcatcccacctgttttgttcccaattctgggtcagtagaaggggagattggggagagcaggcttggtcctgccatgatgtggccgctgctgggatagaagcatggtgtagggacagacaggtagggaggaagaggcttaaggggcttacagtcagatttggcctgtaatgggggaagactgagcctgtgtgggcaggcagggatggcgttgataggagcaggctgtgcgtgcagagtttagcaggcaacaggaaggcttcaccctgtcacttgagaagagacacacccaccttcaccagaggccctggtcagtttgccagagatagcaggtggagcttatgcggcatgacctctaatctcatggcttcaggctccgaggtctccggaattacggagacggggaagggtctccacgcaggacctttacctacatcctgccaggtttctcaagcaatggctgactcatccttccttgcttctgtttggcttccctccctggaggcctggagacccacctccagctgttggttcacatagctgtctctaccttctccactctctctgtacagaaaaaaataaacagacctgggttttggatcagccgttgtgctacctcttttttgtcccctcccaggagcccttttcatcctccaaggggcctaaggcttagagccctagaggctctgaggaggaaaagctgtttacaatggtccctgtcactagtcctgtgaacggggatgtcctcctatgccataactgagccactgcccagagagggcctgataaagagccctctctgggcagtggctcagatgccttctgtctcttcaataacattgaatcctagcagccgacactgatttataaggcactgttcaagctgctggtgacttgaccaaatgcaggagctctgatctgttacagaaccaactaccatctgtctgaggctgaactagcaggagtgggaaggaaggaaggataggctctcaatacagcctctgactattactaggtgggcagtcccctagggaagggtggcttggcagaaacccctgcaaaggtcaatggtttgtggcgagagccactagtgaacctagatagtctggagacccggactgtattggtccagctctgagtggacacaagacctttgtagaaaagctttctgaatatctgtgttggggaaggggtatttgtgtgtgtgacttattaacatactggcctagcattcgtgtaaagcctcaagtccatcagcactacataaacatggtgatgcacatgtgtaatcctggcacttgggaagtagagaccgggaaggttaggagttacaaaccatcctctgctccatagggtttgaggccagcctgggctgcatgagtcactctgccaaagaaaaaagggaaggaaacagaaggaggcagctccttttcggaatgagaaatgatgcaacactttacacttaactgaccacctaccatgtgctgtccctgtaccaggcctagagaaatgatcttacgtttacgtatcgaggtgtggatgtctttgagccagagttctcgacatcccccacaaatttgttgccatccctcacagttggcagagttttactaaccaggaaacccagacctgagagagaaggaagcagacccacgtcgcatagcacagaactggctaagtgggactagaacgttagggcagccaggacacacaggaaaggtcatagcaacagctaagaaaagtactcaggcctgtggaggcaggtggcctggggaaggaagaactagatagctggaggctattgctgctgccagctaaggacagttggcatttgtaggagagctgtctgggaagccatagtgtgtgctctgctgcactgcccaggaaatactctttaaaaaaaaagaagaagaagaagggctggagagatggctcagcagttaagagcactgactgctcttccagaggtcctgagttcaattcccaacaaccacatggtgacatgcaaccatctgcactgggattcgatgcactcttctagtgtgtgtctgaagacagctgcattgtactcatataaataaaaaaataaacacatccttaaaaaaagaattttttattttatgtatgaaagtgttttgcctgtgtgtgtgtgtgtgtgtgtgtgtttatgtatcatgcctggtgcttgagatcagaaggtgatggattccctaagttagagatggctgttagtcaccatgttgggtgctgggaattgaacctgggtcctctggaagagcaactagtgctcttaactgctgagccattgctccaactacacacacacacacacacacacacacacacacacaccccacccttttaaaaatagatttctttttatttatgtgtatgagtgctatatctgtaggatgccagaagagagaatcagaccccattatatatggttgtgagccaccatgtggttgctgggaattgaactcagactcaggacctctggaagagcagccagtgttcttaacggctgagccatctctctagcctcttctcttctcttttctctctcatttcctttccttttcttttcctttcttttctatgaaaaacacgcacacacactttttttttagactaagctggccttgaatttaaagagatctgcctgcctctgcctcccagttgctgggattaaaggtgtgcactgccaccccccacctgtatcagacacccatttttttaaaacaggagaaagaaaatgttagatttttattttacctatcttatctagatgtctagtgtgtgtgtgtgtgtgtgtgtgtgtgtgtgtgtgtgtgtgtgtagagggatagtcttggcatggaagcagtggagtctaaaggacaactttccagagctctgtccttcttctttgtgaaacttaagtcttcaaacttggcagcagatgcctttacccccttagccatttcactttgcttagtcccagggttcagggaaactgcatgacgtcagctctggctgccggctcccagtctgcccagtgacatgactgctgtctcttacttggcttctgccttctctgttgtcttcccgattgaccaccaaggccccagtctttccttgtgagaactggagcttgttctgggcatcacagccgtcaccattgaaactctctatatcccttgtctgggacagaggctggtgatgtctgaggcgtcaccattgtcctcaggctgcctgacatctcagattgaccaaatgtaaagtcactgcactgctgtgttatgtgttactggtcctggagagggggggggggtgttcagggacagattcaccctctgagaggctgcattgtagactagaagctgagattctgggacctgctgatcagggtcttcagatggacctcttgtgattcagtctgagttgctgccattgctggtgcaccgcagccttgggcagggcccagtctctgcacctcactcagttctctgagaagaggcagctgagctacctgggatctccctgctgtgctgttaaatccacacagtgctctacttcctgcttgtcacacctgtgctactgctccacagtgcctacagagtatttgactaaattagtgaattgatttagtttaggacttttgagacagggtctcaactcaggctggtctataactctctgtagccaggtatgaccttgaactcactgaccataactgtttacagatttacaacagccatcattgttgaggaagagggtgtctcctccctctcatgcctcgctccttgggctctattgtagttcagtggtgttttctttaagatcaaaacaaatcctccttacttggtccacccaggaggaagttccttcgagaagataaaagactctttttcttttatgtggctgactgcctaagcttttggatgggtgttagtattgacctacaccccccaccccccactaatgtcggatgggccttcaatattgacctacaccactccccctacacctttcctcctgtcggatggcccatcagcacacacacacacacacacacccgctgagctgtgaagcttttaacaggtcatgcctcttttatttagtgtgtgtgcacacatatgtatcagtgctcctgtgtatatcgtggtgcatgtgtggaggtcagaggaccacttgtgggttggctctctctttccaccatctcagtcctgggcattaagctcaggcatcagccttgctggcagtggactttcttcctcccctctcctgtctttcattcctttgcagtgttggggatggtgccattagctgcccatgctagcctggtgctgtgctgctgagctgtagccctagttgatgaggctgacctggcacctgtgttgtagccaaagtcaaccttgacattctgagtctcctacttccgagttccctcctgcccctcagattgtcacttgtgtctctggaagccactcagccagagttcttcctgagcctgggattcacagacactctgtatctatttgcaaattcttcccccccccccccacagggtttctctgtgtagccctttgtagaccaggctggcctagaactcagaaatccgcctgcctctgcctcccgagtactgggattaaaggcatgcaccaccacgccccggcagtaatgaactttttgaccctcctatctcctatgccctgctagtgacctttcttgagagacacaattaagtatgcatcgtaaatacacaccctgattgttctgataactttaaatcaaaacaaaacaaaaactgctgatcaccgtacagatcagcattgaggctaatttttgcctcccaagaaagttcagttgtgtgtcttcctgcctgttcctccaaggaaaagcctttgccctgattcctatcacaaaagatcagctttgcctgttcaggttcaagtctatctctgtggtagcttgtaacctgggccacatgtctgagaagatgtgcatggctccaagacagccatactcttctctccctcttctcccctcccccctctgcctttgtagaccaggctggcctggaactcacacctgcctctgcctctgagtgctgggaataaaggcctgcgccaccaatgcctggcagctatactaatttttacacctagttagggagaccacacagggataactgtgggccagccatgcagcagtgctgatggtgacctaacttgggatttttggcctctacattagctggtataggcattagccaccatggccattttcttccgtgctgggaatcctgctggggaagctctctaccaactgagccacaccccagcccttgttttattttgagacagcttcttgctgtgtagtcctgtctggcctgcctcagcttccagggtactggggtgactggcggttgtttgttcggttggctttgaacttgatccttccccagtctttcttgtggctcttttgccatgggcaagattgtctgaaatacaatgcaaggagggcttagtggaagtgctagagcagattgtgtgtacacagctctgtgtcttcctacttagttcaggtcccctgcagaccttgggctgcagagcctcggtagccgagggctttccaggaacgcagctggggttggtgcgtttgttaaaacttgcaggaaacagttgcctagtgcctgtggtcaccagcagcactgagtcatttctgctgctttctcttctcccagggacttccttccacgaggatcaggaattgtcacccggaggcctctcattctgcagctcatcttttccaaaacaggtatgagaaatagaaattcagggactggagagatggctcggtggttaagagcactgactgctcttccagaggtcctgagttcaattcccagcaaccacatggtggctcacaaccatctgtaatgggatccgttgccctcttctggtgtgtctgaagacagcaacagtgtactcacatacataaaataagtaaatctgtaaaaaaaaaatttaaaaaaagaaaagagaaatagaaattcaaaaccaagacaaagtagaaataataagtaaagcgagggacgcagaggtagcagctcgtggacagctccctggcatgtcggaagccctgctttcaccaccaccaccaccaccaccaccaccaccaccccccaccaccacccccacccccccaccccccccaccccccccgcccagcagaatgaacctggcctgaaggtgcatagctgtaagcccagcaccgggaatgtgagaagggctagacgctccaggtcatcctgtgatgcatagagagctcagggccaccttgggctacggaagagcatgtcttcaaacagcagcaataaattgaagaacacccttctacaaccctgagtccccaggcaggaccaggctacctggtgcccacagttactttctgatttatttattttagtttattttatttttttgagaagtcttgcctggcaaccagaattgctgccttgtactttctgtatggtcatagaccagccccaactggaaggaccactgtgcctggtatttgaggcttgatccatagtctcatgctgccagttgctcccagtatggcatgttaatgaatttacagatgggaactgaggcccagccaggttacctgcgtttttagggggccactggcctgggatcctcagccaatccagtgcggcctgacattccagggccctgcagttgttctcctgattctaccagttaggcgtggaggggaggctccagcgtaagctgctggtttggtgtgccaaccacaggatgggctgctgcctcttccatcaggcttgtgctcagctctgctgccataggccctgacctctatccctgtgcttgctttcatcgccttcaggggttccacctcaccttagaaatccctattttgcactttttccttttggctttttgaggcaaggtttctctgtggctggcctggaactcactctgtagaccaggctagcctcgaactcacagtgatccacctgcctctgcctccaagtgctggcattaaagctgtgcaccaccactgcatggcttacttttgtactttttcatggaacatcagctgccactgttgctgctcctggtccctggccaccctgggaaagcctgcatggctgcagagaagggagccaggctttcccgggctcctgtcttccctttgtaggtgcctgcaaagtgcagacaattggaagacttggttgtgtgccaggcttgttatacctgcaggttggctgctcgtcagggcagctggtgcccgggttagcctctggttctcttggacctgagtgctaggataacagtgctaatgagccactgtatgtgactttattgtagttttatttattttgaaaatttttatatatttttaattgtatgagtgttttccctacatttctctctgttcccatgtgtgtgcctggtccctgtagaggtcagaagagggtatcagatcctcaggccttgcacatggtaggcaagtgctctgacatacatccctagcctgttgcttgcttgcttgcttgtttgtacttgttagctcaggctgacctccacttgtatctgggggtgaccttgagctcctgatcttcctgccttccaggtgctgggtgctgggattataagtatgtactgctctgcctggttcatggggtgctggggatggaaccccagaattcatgcagactagatgagcactctgagccatagcctgtcccaccaactgtgtattttcacttcttttgagacagagtctcactttgcagctaggcaggatttgaactcattgtattccagatgggccttgaacttttgattctctatatgaacctcctgagtagcggggacagcatacccagggcctgactggcatgtacagggaacactggggtgctggtggctagcagacctgttgtgttttgagattccttcattgccagagtctactggaatcctgaggtctagggtagggctggggagaagaccatgaaaggaacacctgcccagagctggcctgcataaaggatcaagtcagtccctgactttctccatcttcaagggaacacaaacgtcccacctctgcctgagtttactacttagatctttgttggaacttaccttggggataagaccagacctgtatagctatggtaagaagggaccttagactagtgccaaagtgggtcacatgggaggaggcatggctgcttagggcaaggtgacagttataggggcatagttggatctttttgggctgagaggacaggtgtgtctctcaggtgtgttctgtcctgtgtcccctaagcttgaatgcctctgaccctttgcctaatggtggcattgcccatattatgcacagccttggtgggacattggaaagcagccctgtgtttctgtcacatgtgttatgttgtcaccaggctgccccacttccccacctctggcctaatagatgctgcagacatgcattcaccacactcctctggctgaaaagagtgtatctgggctgtttagagagcagtagtgtgtgcagaggtagagaggccagagagggaggtatagctgagccccagagatcatgagttcctcacactgaagcttgggacctaagctacagagccagggatttgggcagacagctgttcactcctgaaaggttcaggagtctaaagatgaggcttgtgaggccaagcgtggtcttccccttcagtactttagacacccaagtgcttgggagcttcttgttcagactggtctgttgagccttgagtagtagctggtactgtttaaaaattacctaagagcacaggcaccaggaagcttagtggcccatggatgaaccctgcccttacccagaatcatcctgctaaactctgggtacagggcttttctctcccatcctcagccttcgccttgacctggcttggtctatgatcgatgggccataaaagagatgtcagtacatagcctgtgcttgccagagcccagcctgtcttgctctccaaacacagccatggctcctcaccatgttccacagggtcacttgttagataaaactgtgctttcttgagttgaattttgtttaagactgaatctcatgtagctcaggctggacttgaatttgcttatacctgaagctagggtctaagaaaaaactgagcttggagcaggtgtgactcaagttgggggctagttgtaataactgcttcttgtaatcatagtagcagtacatcttagacaaggtgacagtccctactggtctcaaatctgaagaagctataggctaaacaggagtagtgaatgtgtgctaacgctcctgtgtaaggctctgaaaaatccttcatgcacaaatcctttagactgttctgtttgtttgtttgatttttgagtcagagtgttgggtaggccagcctcaaactcaatgtgtagctgatggtgagcttgagcctctgccccatcctgctgcagtcacatgagcatgccgccacatggggttcatgtgctgctggcatcaaaaggttctctaccaaactgagtcctatcccgtcgtgtttttcagatctattgttgtaaggtgcatgcgtgtggatgcgtgtagagtccgtggtgttggatcctcctgagctggagctgctggtgtcatgagcctcctgacaagggtgctgagcaccaggcagtttccctcaggagcagtagtcagtcatgactgtcgggccaactctccagcctccttgtggtttgtttgagactggaacttatggccttcattcgaaccctcaaggttcacagaaggttcacagatcctatttggttgttggttgtgtccgagggtctttaacatgtgtctgagcacccaggcattcttgtccagacatgcatgccaataccaaatctgaagaaggcagaccttgtgggcgtgggcatcaggagcctgtcaccatggtttcctctttggtgggctgtgattaagtcctgatccttgtgcttccatcttccaacacctacatacatgcttaagaagcagaagcaggtgctggccagtgtagtgagtcaagcccagcccgttatagtcacacccatgcaggcaggtagaagctccttagaaacccccttcccagttcatttcctgggcacacagtgatgctgcccttggggaactctggcaagaaggctggctggcccaccaggcttagcagaatgtctgctgaccccggctcacctttggtttcttcctttatccctgctgtgtaccaggccctgggctgaggcctgtgtagcctgctaagaggccccaggggagaacctaactggttcactgtgcagggaagggcagggagaatccactctgctgccagtggggatgtggctatcagaatgctgcagtgctggcccaggggccagtggaacatggggacctggatgagcagagctgtcaatgacatagaggtatggcctgggctactgatgaccaactggcaggcagctgaatccagcacacttttttttttctttgagatagggtttctctgtatagccctggctgaccgggaactcactttgtagaccaggctggcctcgaactcagaaatccacctgcctctgcctcccgagtgctgggattaaaggcatgtgccaccactgcccggatcacacttttttatataacatttgagcacatgtattaacaggggtatttttggaagcagggtctctttctgccttggaattatgtttaaagtgggctgactggtctgtgagtcccatgtgtcttcccagcactaggtgacaaacattcactcccacatctgtcttatttttgtgagttctgtaaaccaaatgcaggttctcaggattgcatagcaagcactttaccgactgagccgtcagccttgagagcttcttttcaaggcaaggctttgctctatagcccaggctggcctctaactcataatgctgcctttggcttccgactgctcctgcaggtatgagctgctccagcgagcttaaagcgggcatttatgttaggacctgccaaaggtattgtgtggcttgtatcagtggcatgccctctctggattctctcttgtgttaagagagggagatgacacagccttttaccccagcatttccacattgcaccttggggctctctgctcattaatggcagagatgttggctgcctctcttctctgtcttcacacacatttgtttgtctgttctgctgatcaaggcagttgaggtgaaggtagggccctccagagctaattacgctaacaattgtagcaaacactgggtgcctgcgagctgctgctgctgctgctctgctgccaggttccagccccacaaacagaatgcagatgataaaggaggccaaggcagaatgagtggcaagcagggagagggagaagggaaggagccagcccagccctggccttggtcctggctctgctaccttaccctgccaccatctcacatttgctgactgccttgatccggaagagaactgaagttctgtgggggctgggtgactgatccatttttagtggcagctgcaggaagagtatttccgcttgtctcctggaaggccagagcacggctgcccttgggaagtgggtcagctgattgtgtgtgggctgagggggagtcccagagcctcctgtccacaccctcactggccctgcttctagcacccatgctagacagagccggtcctgctgaggtcatcgggtttgagagtcacttcctctacaaggtgtctgacctacccagtcccagcaccaccccatgtcctcctgagcctcagatgccatgtctgtgtgtctctgtgagtgtatgctatttgtgctgatgtgcacctgcctgctgcagagcctggagaagggaggatgtcaggtgccctcttcatccctctccacttgttcctgttttgtttaaatcagtataaccttggcctgccttgaattcatagcaattctccttgcctctgcctcctgagtagtaggattaaaggtgtgcaccaccatgctgggacttggaaaccagcaagccccagtgagccgcctcttccccatgcccttttgggctgagggttatgtacgtttgtgggatgcctggttgtgggtgctgggatccacatttctacccttgagtttgcacaacaaacttctgagtcatctctagccacatgctttgtagttcttaagtcagggtctcacagtagcccaaactggtgcctgccagtctcacattcctaaaccctctgcctccacatccctagatctggaattacaggcctggattacaggcccttgttaccatactgctgtatgccatgctgggtgccccaggctccttgcatgactgccaaggccctgcttgtggagagtctttcctgccaggcattctggccctgagctctttgcattctctctctttccagcagctggccctgtggtcctgtactccggatggccaggtgactgtttcttagatgccccacacagcactcatcctggctccttctccctgcaggagaccttgctgcctcagagtgtcaaccagagtgtgttcttgcaacctttgagacaagacttggcacctccccagcctttaactgagcaaatttaagcatctagatcccagcctggcagctgatgccttcagttcaaacacaaactcctgctcatctttttgtcttttgtcagacagatttcctgtgtggcccagttttgtttgttttttttttttttctgagacagggtttctctgtgtagccctggctgtcctggaactcactctgtagaccaggctggccttgaactcagaaatccgcctgcctctgcctcccgggtgctgggactaaaggcgtgcgccaccatgcccagctctgtgtgacccagtttatctcacgctccttcctcggcctcttaagtatgaggtttgcaggtgcatgctgcagtacccagctcttctcactgaggtgatggggtctcaagtgtcactccatcccggaccacacactgagctccactctagcctgggaccaaatccctttatcaaaaagaaaagaagctgtcagtaaagagttagtcacaaagtatgactcagtactcttataacaggttggttgttggggtgcacacttgtaaccccagcgctggggtggttgagatggaccggctgccagccagcctgggcaagctaacgagcttaggctctgttcagaggcactgtcacaaaagcaatagaggaaggctgtgaggctgactctggccaccacaggcctgtgtgcttgtgtgcatgaacacgtcatgcacagaagcagagcagaaccggtagtacacactttttttttctcgatttttttcgaggcagggtttctctgtgtagccctggctgtcctggaactcactctgtagaccaggctggcctcgaactcagaaatctgcctgcctctgcctcccaagtgctgggactaaaggcgtgcgccatcatgcccagctaatgcacacttttaatcctagcaaagatgaacaaaggagtgcaaggccggagctgcttacacagtgagactatctcaaaatataaaccaacccaataaaataagagaaagaatgaacgccgtgttcctcaagttctgcaggactgaagtggttgcatttcttcctctgcctggcacatgcctggcacatgcatgacactgcatgtccttgtgtattctcctttccctccttctcaccttgaacatctttttcccataacagaatatgcggaatttttgcactgcaagtccaaaaaatttacagactttgatgaagtccggcaggagatcgaagcagagactgaccgggtcacaggcaccaacaaaggcatctcccctgtgcccatcaaccttcgggtctactcaccacacggtaaaggcggggttgggatagagggaagctggtactctgttcctgtccagtagggtcttcctgagcatttgagaagagcatcctggactaggaaggcttggacatggtgggcaagagtgggcataggcacatgcctataaccccagctctggggaggtgaaggacagaggacagaggaagaccccatctccaacaaaagcaaaaaacattagacccaggctcagtggtcaggcacttgtgtagcatgcatggaatcaacccccaggaccacatttaacataaaaatgaaccatgagtggtgttgtgtataccactaatcccagcattcagtagacataaagactgtatttgaaactaatctcctctaaacttttgtaaaaagttttgttttaattttttttaaattttactttatgtgagtgtactttgcctggatgtctgtatacataccagtcacatgcctggggtgcctgaggaagtcagagagggagtcagacccttggagctggacttatcagtggttagaagccaccatgtgggtgctgggagttgaagccaggtcctctggtaaccactcagctgcattctctttactgtagttctttacattttcttttctttgtgtgcatatgtggtgtcacattatgtggtgcacatgtggaggtcagaggacaacttgaagaagtcaattctctcctccttccatgtaggtcccagtaatggaactcaggtcgtcaggcttagtggaaggaatctttacccactgaactctatcactgaccctatttattttctgtctctccctttctttcaaaacacagctatgtagcccagactagcctcgaactgcttctatggtccagttgaccttgaattcctcgtcgcccccccccccccccgttcctacctccccagtgctgcacttatagacctgtgcacccagacatggtctctgctgtgctaggaatgcaaagcagagctttgtggatgatgggtgggtcctggaccacctgggcttatagcctgagctacatggtgagttccaggccagtctgggctacataccgaacccctgcctcagaaatgaagaaggtatgatgggaagacacgtggcttgggaagtagagtgcttgctacacaagggaaaggcttgcattctcctatagcagccatgcagaacgcagcactgcactgcactgcacgtctttaattacggaactctgcaggtctgggcagatggtccatccctggtgctcagtagctacccagtgtagctctgggctcagtgaggaaaccctttctcaaaagaagccagcaagatagctcaatggtaaagacaccttctggcaagcctggtgaccaagtttagttcccagaacccacatggtaaggagacagctgactaccatggatcctctgacttctacttcttgatacacaaacacactcacacatactgacacaaatgcacacttacacacatgcgcacgcacacacacactctccttctctctctttctctaaaaatatattaaatttttaaaaagtataaatatggtagagaacaattgtggaagatatttgtgtgacctctgacctccacacacgtgtacgcatgagggtactcacccaacacagaaagtacacagtgaaatgtgttctagtaggagtgttaggagtgaccccaggtgtgactccccttgggaacttgctggtgtccactgggtttctacaggctgtgtgtaggctctgaactctaaatggggctctcaccaccttgcctgttgatttccccctcagtgttgaacttgaccctcatcgacctcccaggcatcactaaggtgccggtgggggaccagccgccagacatcgagtaccagatcaaggacatgatcctgcagttcatcagccgcgagagcagcctcattcttgccgtcacacctgccaacatggacttggccaactcagacgccctcaagctggccaaggaggtggacccccaaggtaaccgcccgcactgggcagcagccagtagtgctgggtcccctccctgaggtgctttctgtcacagttgtgttagaagcctcctgagattcagcattccctgtaaccacatcactataaccaacactgtgacagagccaccgtctttttttttttcttttttttccttttttttttaattaggtattttcctcatttacatttccaatgctatcccaaaagtcccccataccctcccccccactaccctccccacccactcccacgttttggccctggcgttcccctgcactgaggcatataaagtttgcgtgtccaatgggcctctctttccaatgatggccgactaggccatcttctgatacatatgcagctagagtcaagagctccggggtactggttagttcatattgttgttccacctgtagggttgcagttccctttagctccttgggtactttctctagctcctcccttgggagccctgtgatccatccattagctgactgtgagcatccacttctgtgtttgctaggccctggagagtcactgtcttttaaagagcatacagggttagttaggctcctggtttcagagtgtcctcttgtgattggtggtttgttgctgctgggcctgtgagcagataacactttcctgtgcacgtggggagcaacatggaataagaaaaggtcaagggaggcagaggcaggtggatttctgaatttgaggccagcctggtctacaaagtgagttccaggacagccagggctacacagagaaaccctgtctcgaaaaaacaaaacaaaacaaaacaaaaaaaagaagaaaaagacaagggatccctttaagtgtgatgcttcaagtgaccccagcgacctacaagcttcaaggagacctcacttcaccacctctcagtggctctcctctacagacagcttagcacacgggcctttgagagacatttaagacccacattttagtctgcagatttaaccccagtgaggggttgtccgttcagaggtgcccagcttgaatgattatctgggtttgatcagaggtgcccagaatgaatgattatctgggtttgatctgagaatctgttaagagtactttctgctggacatggtagcacacgcctttaatcccagaacttgataggcagtggcaggtggatccccttgagttcaagtctagccagggcttcacagtgaaaccctgtctagaaagggcttggtggatggcacttaactgttctttttatttttttggtttttcgagacagggtttctctgtatagccctggctgtcctggagctcactttgtagaccaggctggcctcgaactcagaaatccgcctgcctctgcctcccgagtgctgggattaaaggcatgtgccaccacgcccggctggcacttaactgttcttgcagagaacatgggttcggtccccgcacccatacgatgcccacaacaatctgtggtttcaattcctgggatctgatgccctcttctggcctctgcaggtacaacatggtacacaaatatacatgcacaccaaacactcatacacacacacataacttagttgacctaatggcatatataaatcaataagacccagtctttagcagagccctcccaggacacagttataattccaggcacatctgtctgcattgtgcagttgtatctggtgagatgagaacgtctgggcctggagtagctctgttggtgtgtgcctgtcaagcaggcaggatcccagcactctgtgacgctggtgtggtgccgcacactagaggtgaaggcagggggatcaagatttcatcatctgctacactgagaggccagcctgggctacaggagaacctctctcaaaaaacaagaagaaataaaacctggaaaagtagagaagggcgcacacaccctgggagagtccgggtggggttttgagcagaggaggcctgtgaggccgcagaatgagctgcaggtagggctgtgtgccctcgtcaggagcttcttagcccatccagccgtgctgtccgctgtccacacacagtgggcgtggccatggcaaacattagcagggcagtgcaggcggtcaccatttgtgagcacatttttcttttctttttttcttttttcttttcttttttttttttttttttttttttttttttttttttttttttttgcctttgaaaatactcatggtttttacagaaaagttaccatggaaaaatcaggagcctgcaaagctccctttttccatgtagatagcgctggccacagttcctttcaaagtttttcttacacgtaaacttattttttaagaggacagtctctccttatttctgtctgtgcctctgtggtcattgcttttgttgattgcaacttttctttgtttatttttcaatcatggtttcagaatgtagccctggcttacctgtacttcactgtgtagaccaaggcagtctcagtatcagattctacctgcctccccctcccaaatattaagattacagcaagccaccatgccttgccctactttatattatagtgatagtgtgttactctgtgggcaggccccattgtacagttaccctctcctcaccctcactgtctctcttctcagcagctggcttcctgggcaaccatggctcaggcccctctccactcatttctctccatagtgtggcctcttccctatcaactatgaattcttataggtgtacccacttcacaatagtgccagctaaggtagtcacctgtgatggccaagttgatcccagtcttctgtgggcctccattagcctctgtgtgtcttgcacacatggaacatacaggcaacacattcacaggtgtgtccaagccggtgcctgggttccttatgtatcaggtggttcttgttagttaggcaggatcttctgtggtccagattggcttcaaactctgtttagtgggagatgaccttaaacttgtgatcctcctgagtctacctcccgagggctgaggtcacgggtgcacaccttgcaggctcagtgtaaccagatgtttaggaaagagcccaggctccttggcaagtgctccgcacctcacctggcacagggtcgacctagggtttctgaatgctaaggcaagtccatggagtacatgggcttctaccctggcttggaagctcagcttataaactgctcgggtgaaccgtggtcaggaccccaaactagggatccgggagcaagggaggctgagaagcacagtggatttatttggtggtgtggctttgctgtgggatgtgctaactctggttgacttggggagaactttgatgggctggtccttggagtagtaattgggccaggttgctcaggttcaaatgtgatgttgagaatgggcaggggggattctagtttccgtgatgttcattggttctgctggggtcttgttatgcgcttaccagtgttctgtcactagagcagtaactgtcattttatactctgccaacagttgtccaggcaaaagataccatggcttgtccctgtctcttggactgtagagggccccaagggcccttgatcatgatcccttctgacctctggactctttcaggcctacggaccatcggtgtcatcaccaagctagacttgatggatgaaggcacagacgccagggatgtcctggaaaacaagctactgcccttgagaagaggtatgtacacagggctggggtgggccacagatcaggggtgtatcttttgtggttctcctctggacacagtgtttctacattacagctgttttctggaacactaagaagcaggtatgcagccacactcgtgctcggtacttgctgtaatcccagcactctggaggtggggttggggtcaggaattccaggtcatcttgggctacatagaaagttctctgtcagcctgggctacatgagaccttgtttcaaacaaacaaatacagaatgtttcatcagaaatagattgagttactggaaatgagtctcagtggttgtattactcagggttctctagagtcacagaacttatagatagtctctatagagtaaaggaatttattgatgacttacagtctgcagtccaattcccaacaatcgttcagtagtagctgtgaatggaagtccaaggacctagcagttgttcagccccacatggcaagcaggcgaaagagcaagagtgagactcccttcttccaatatccttatatggtctccagcagaaggtgtagccaagtttaaaggtgtgttccaccacacctttaatcccagatgaccttgaactcccagtcttctggaatccatagccactatgcctcaagatctccataccaagattcagatcagaaacttctatctcccagcctccaaattagggtcactggtgagccttccaattctggattgtagttcattccagatatagtcaagttgacaaccaggaatagccactacagtggtatagcccttgctcagcacatgcaaggaaccaggctcattccccagcttcccaaacaaagcataacaaagagacactgctgctgcttgagtcttccctagctagtggaggctgggggatgaaatgcaggaccagaaagggagtcaactacatggggcagtccctgttgaactgcacctccagcccactcatacctgcccttgaagttcacgatgctttcctcagctaccttccatgccattcacacccttggtggggctcagtgctgccctgtgcccgctctcgtgagtgatttttggaccagtctgtttctagtctccacctggccagctcagcacaaagccatagcaccatgtccttcctggcgtaacgcataccttgaccttctttcttcaggctatatcggcgtggttaaccgaagccagaaagacatcgagggcaaaaaggacatccgggctgctctggcagccgagaggaaattcttcctctcccacccagcctaccggcacatggctgaccgcatgggcaccccacacttgcagaaaaccctgaaccaggtatagcggaggtttgaggccaccaagggctggtaactacagctaaatgggaggttgctgtagtctggactcctgcaatctaaccctgtgcagcctttgagcacaaatcctggcctggctttcccatgccttctttctcttgtccatcttgcccttatatttcctagggtaggggtctgtgaggccatgtgtgtccaggacaaggtacatgccttgtgtgacaggatacagtaaacagtatccacccactgctctgtgcctcacgcctccttactcacagtgacgacacagtgagaaaactggagcgcctgcctagaaccccctagtgagggtgtgggggtgtggcttggtggcagagcacctgtttagaaccccacagtgggagtgttacttagcaatagagtgcaggcctaggatgtggaagccctgggcttcatctccagtaccgaataaatccagtgtggtggtgctactgtaatcccagcactcaggagctcagaaagccaaagtcattctcagctatgtgatgtgggataagtgagagtctacctgagtcaaaaaacaacagcaaagacctctttgctcatagattgggacctgaagagagagccattgtccttagggaatgtccactggaaagccagctggtaaacgagccaccagatgaattcctcaagtgttagctgtggctggaggttagtgcagaatgaagctgagagcagcatccctagagcaggtgcagcaggcattgggcagtgtgtcttgtaggaagggataaagggggctggagagatggctcagtggttaagactgctcttccagaggtcctgagttcaattcccagcaaccacgtgtaatgggatctgacaccctcttctggtgggtctgaagacagtgacagtgtactcatgtacaaaaaaataaataaatctttaaaaaaaaaaaaaaggaagggctaaaagcagagaccactggacctggccaaagtgtggaggggcaggaaggaaaaccctccacagagcctggggctattgagaaccagaggtgtatagcttggctggcccccctgaattaagaggtgctctgtggagcattgattgtttgtgagctcagcgatgtggtccatagcagccacctgctgctcaggcctgaggacttcatttcagtccccaaactaaaggaacggtagaaggagacagtagactccatgttctctgacctccacccaggcaccctaacatgtacacttaccccccaacacacgtactcacagaagtaaataagaattctaaaaggcccacagaacaacctggctcccacagacttggggctcgtcacaggccggagcctctgcactgctctcctctagcattggtgtgttcagaatctttcttttccctgctgggaatccacctgctatgcagtggggctcttgctctgtccctcccctccctcagtctcccgcagatgtggccccacgtcctgctcaaagcatatacctttgagtcttagttagagggccatcccaggcctgcacactggtcccaccatccccatccttgggtgtcaactgacatctgctacaggctctctgctccacttgttccagcttctgtccccgtctcaatgatttgtctgtaggtttgtggctctctttggattgcacccaagactgagtattctggcctgagggtggattgtgttctttcctggcctctgggcctgtggtaccctgtctgacccttaccttttctatagcaactgaccaaccacatccgagagtcactgccgacccttcgcagcaagctgcagagccaactgctgtccctggagaaggaagtggaagagtacaagaatttccggcctgatgaccccacgcgcaagaccaaagccctgctgcagtgcgtactcactccccttccccttgcctggcccagtcatcctgctctccattcaggcccttgtcctaccctgcccctgcgagtgtggggtccagtctctggacatttaacatgacactgtcatttgtagggttcatgtcctgaaactgtgtacctgcattacaaaaaaacagggaaaagtcacattgtttcaacaagttaccattgtgtgctaatcccagcattttagtaagttgccattgtgtgttaatcacagtgttttagtaagttgacattgtgtgttaatcacagtgttttagtaagttgtcattgtgtgttgtgcctcattcgttgctgtagtcctgtggcctgtggcactcaagttggacacagctgaacaaggtatgttatctaagcctgtcatcctgggccttaggaggtggtggcaggaagataaatcaaaacttgctgagctgagaacgtggatgagcaagttaaagtcgagggcctgagctccatctcctgacctagtgcaaggagagaaacccttcctccaagcctgagggcgtgcacccgtgatcccgtgggtcatggggagatccagtctggggagacagactgagctccgccttcagggagaaattcagtaaagtagagagagatttaaagacagtggacagtcacttctttcctgcgcacgtgcacacacacacacacacacacacacacacacgaaagagagagagaaaacaagacactgtttaaatgaaaaaaaaagaaaaaggagagagcaagcaaatataccatgatcaactgggtgtggtggcctggtgcaaacttcagccacttggtaggcatttgggagtcccttccttccttgcatcatttagttccaagaggtcaaacactgtggccctcagactcagtgagaagtacctttacacactgagccatcccaggaccacacctacccccaaaatatgttctttttcgttttcctttttttttttatgcttacatgaactgttgatgccttgtgtggttgccaagttctccggtgctcaccctgtcacctgctctgcacagatacaagctgagcctcacagtgcagagccacttagtcaagatttcccagcctaccatgttgtcgctcctgtgggaccgaggtagagaggtagcggaaagtaaagatgctcctcagccggtctggagcagctgccttgacagacagccatgcagtcaatcatgggacctggggaaggactgcatagctgtgccctcagaattccatctagaattttagtattaacagatctattgaggtcttgagaacaaccaggatagtgcccagcacttaccaaagtagcgttgatactttacacattggctgttaaacagtgatattgaaaaagaaagaaaaatggccaaacatggtggcatacatgcctgtaatgctagcatggaaggcccagacagaagggtctgtagttgaaagccagttggggttacacagcaatacctgtattcacatacaacaatcccatgaaaatgagccacaggactgacaaggtggctcagcgggtaaacaggcttgctgccaagatagcctgagttttatccccaagacgtacctggtagcaggagagaactgagtcccataagtctcctctgaccccactcatagactgtggcatgcctccttccaatacacaaatgtaacttaaagcctttgtaagagcagagcttatctcttgattcatgtaattacagggccgtgcagaaaactctgtcttaaccttttgccttttctttcgtgaaggatggttcagcagtttggagtggactttgagaagcgaattgaaggctcgggagatcaagtagacacactagagttgtctggtggagcccgcatcaatcgtatctttcatgagcgctttccctttgaactggtaaaggtaggtgttcagcctggagttaagtcagacactctcatgcttggtctttgtcttggccttaaaaattcttacaaaccatgatgttacaagctcctgcatcatgtcagaggctgctgtcagcaaacttggtctgaaggacatgtccccatcttcccagatctcataatttagaagctctgagctgaacaggaggaaagtccatgtttcttccactggtggagggaagaaagatacatgggggccaacgagatagcccagcaggtaccctctgctctgcccgactgcctgagtgtgatctgcaggcagaagcacagaaccaactgcaggttgtcctctgacctccacacacacaggcactgacaacacacacaaatgtgcacagagggctgaagagatggctcagtggttaagagcaactgctgctcttccgaaagtcctgagttcgaatcccagcaaccacatggtggctcacgaccatctgtaatgagatctgacgccctcttctgatgcatctgaagaccgctacagtgtacttaaataaatctttaaaaaaaaaaaaaaaaaaagtgcacaatgctaggcttacaccagtaactgcagagttgtgtgagttgaggcaggaggagactgagtttgaagataacatggcctgcaaaagaccctgtctcaaaaactgaaacaaaaacaaaacagaaaagtaaagcaaactgtctttgtgtccttttcctttgaggccgtagctctcacctcataggtgtgtgtatgctagtgtggagatcagagaacagcctgcagtgtcccctaattggtttctctgtgttagccctggttgtcctggaactcactctgtagaccaggctggcctcaaacctagagactgcttgcctgtgcctcctgagtgctgagagtaaaggtgtgcactgcctctgccacctagccactacgcacttatttttaaatcaaggtctcattgtaactcatatttggctgtgtagtcatgcttgatcttgaccctctaccccttatggtaggattacaggctagtgaatgtcagcacatccagtttgcctgtgttgtagatagggcccagagctccacacatatgtggcaagcatccggcgagcatgagaaccgcagcctggcttagccttattttcttagacaggatgtctcacctacctggaacggtaccagtaggttaggtctgctggctggaaagtttagtgttttacttatgtttatgaacattttgcctagtgtatgctgtgtgtaccatgtacatgcccaattgcctacaatgaccacaagaaggcgccagatcccagggaatggagttattgatttagtgagccattatgtgagtgctgggaactgaacctgggtcctttgcaaaagcagccggtgctcttaactgctaagccatctctgcagtccagtatgcccagatacagccatctctgcagaccagtacacccagatgcactcaggtctgcaggcttagtggcaggtacgctaacctactgagccatctctgtgaccttcagttgtttgtaagtttgagcttgctacatcatctttgtgagatacactggtgaatagaaacagagctagccactgttcctgctgtgaggtgccctggacgtggctccatgtgtggttggtttcttctcagatggagtttgatgagaaagatctacgaagagagatcagctatgctattaagaacatccacggagtcaggcaagttccatggggaattcccatgttctcttcctgtccattcatgtgggccccccaaccttctgagtccctggctagtgaggcctttagaaggttcatggatagtttccagaactgtataaacatcttagtcttcaaggacagagaaagtgcgtgtgttttggctgtgactgtcctaggtctcttgattacttgtgtcccctaggatacatgaccttgttctgtgagatctatagtgaccagaggttgggtggaaccagacttgaatcatgcccacaggccccatgtcatcatgggtcacttgtgggcctcctggtgtcccctccttaattgcaatcaatgaagtataccctgttctctagcgtcccaagatccttgggttaccctgagggctccccagttggggactctctggtatcctacccccagctttaatgggatagttttccttgtcagggacctagccatgcttagaaagttgctgaccacaccctttgtttctctctgacttatctctccctgcttccccactgggtctggacgtttttagaactgggctcttcacgccggatttggcgttcgaggccattgtgaaaaagcaggtggtcaagctgaaagagccctgtctgaaatgcgtggacctggttatccaggagctaatcagtacagttaggcagtgcaccagcaaggtaccacacctgcggacgcggggccgcaggcttggtatcaggccccaggttacctcacttccaagaatcctacagggatgggactgtgtcagtctggacccactcaggagggggtgggggaatgctgggcttcttagaaccagctagactggtgacaggtctcctgggatcctgataccaagcccagcatgcgtatggtggaatggggatgtcatctggctgtgttgctttgctgcttgcgagggaagcagatcgggtattcctcttcagctgttggaggccaaggctcccgaagcttcactgttatcccccttgaaaaatgacaaatgcctgcttctttggagtgggtcacttctctgttcccatggcactgggaagaacataaacactggtggcatactgagagctcagtgcccagcttcacagagctacttgggttccacagtggctctgccagccttcactgtgccctgtgctgcccttgttccatccccacccctcccaggtaactccacactcagtcctcatggtcttcaatgaggactcatgcccactcttcatgggcaactgcctccatttggcccaaaccaccaggctcctctgggaggactgtatcctgggacagcacgctcctgccaagcagcgggagtgcttgaggggaaggagagggcgtctgctagtgcaacctcgacattgtcagctctgaaagggcagagagcacctgcttcagctgcctgtcctactggagcaaagtccccatttgcttcctcagcttctccagtgggaaacagagcaaaataagccagccaagcaagggctggggttagcgttagggtgagtttgtagagtgcttggctggctgcacaaagcttggggttccatccttagcatcccatagaccagatgtggtggctcatacctgtgatcccagaactggatccaggagttggaagtaggaagttagaagttcaaggtcatcctcagtatatatactaagttggaggctagcctgtgctacaaggccatttcaaaaactgaagcccagaggggctgctgattgacaccctctggtcccttatcacagacgtgatgatttaagattcctcacatgcctttggccctatgctgagtgtcctgtagcacttagccttgagctgtaggccagcctatgctcccccaggccttgcttatcccaagctgcttccaggacagatgtgggtgcaggcttttctgcgtatgcactaaccagctcattctcctttcttctctttctctctttatctcccgtcctatgtgtgcatttgtgcgtgtggcctgcactacccctgggtgtctctcaacctcgtcccgccctcctccgcatgaccaggacggggctcttcacccccgacatggcctttgaagccatcgtgaaaaaacagcttgtaaaactcaaagagccgagtttgaagtgtgttgacctggtggtgtcggagctagccacggtcattaaaaagtgtgctgagaaggtaacaggtcttgctcctcctgtccctcatcaattcccacctatcatctcttcattggtgtcagccctgagcatcgggacaaccttgcctacaggggacccctgctgggcacagtgccatctggaactttgcgtggcaggctgagtgggggaggtacagcgcacacgtcccaccctctcagttttggggtccagttgggaaacctttgccatgtccaagcacagtcacatctgtgaaaccattctcacttctagatgtcaccccttgccaccactgtgtcctcctgtgtgttcctgcattggggtgcgtttagcagcatctttcctctcctggggaaagtgtggctcgggtcactcactaccccagataaaaagaagggagtctgaggattggccacatggctgccaaattcacagagatcagggaatattttccagcaaagggttccctgagctcagcgaccccgacgctgtgccttttatcctggagcagggcactatgctcattcgaatgcccagtgagggccatcagtttgtcctgcctgcacctagtgctgggcgggagggggctgcctggcacctctgggtagtaagtgtcattcaccctctctgccactttccacctccaatcgctggctttgcaagcggctctcagcatgcacggcaccctcttgcctttcccactccacatgcccctgactggcctgcccttgtctcccggctcccctcccctcccctggcctggccatgaagaccttgtcagctcagcccttgtgcttgctccccagctgagttcctacccccggctgcgagaggagaccgagcgaattgtcaccacctacatcagggagcgagaagggagaaccaaggaccaggtattgtaccagttctctgtttcctctttgttcatgtctagcaaattttgagaatttagttttgtgaagtgataatatttttctttaactcaacctggcttccaaataaattagtcagagactctaagatgtacttaatcaagctatatgacacaatagctaagtagttattaatctgttctaatccttgaagctaatctggcgacctcccagccatagtctccaaggtacttgtatcttgtgtctggctctctgctccaatatgttcctatatgttccaatatgttcctatggtgagcatcacagagattcctctctttctttaccctcccctggctgaggtcagaagtccagccatacactctccagtgctcagccattggcagatcagcttttattgagaaaacagagaataaaccaacaactgtatacacaacattgagacaagagatacttagaatagacaacagtaccatgtccagattgccaccagatatggggacagaaatcagcatttgaataagataaggatatttatttatttatttatttatttatttatttttgtttggttgtttggtttttcgagacagggtttctctgtatagccctggtgatcctggaactcagtgtatagaccaggcttgccttgaactcagaaatctacctgcctctgccttccaagtgctgggattaaaggcgtgcaccaaacatgcttacactgtgaggttttggttttggttttggttttggtttttttttttttttagatattttctttatttacatttcaaatttcttagtttcttctgaaaatcccctatcccctcctcctccccctgatccccaacccatccattcctgcttcctggccctggcattcccctatacttttgcatagaaccttcacaagaccaagggcctcttctcccattgatggcccactaggccatcctatgctatatatgcagctagagccacaagttccaccatgtgttttctttgattggtggtttacagctgtttatctcgcctgtgttttccatagcatgcacgtggaggctggaggacatctctgaggagggttcttgccttctctttatgcagaggttctgggggtgatgtgggggggtcgaacttagttcaccaggcttccaaggcaagtgtctttatctgcggagccatcctaacagctccagatccattctgaggcctaggaccatgatgcggctaatatagcacttgcctagcatgcgaagccctagtttccacctccagcaccctgtcaggatgtggtgaggcacaattgtcatcctaacttgggagcagagttaggaagatccgaaactcggtgtcatcccttgactacataactgagttcaaggccagcctgggctacaggagactgtgtcgcctaccacaccttcccaaaaaaaagagagaaaggaaaaaacgaattattactatctgtggccaccaacctgtatcctggcctccccataatgcattgagaagtcttccaccatgattgttcaggtggcattaatgtctttgtgcaactacagattagattgttctatacatgcaactaaatctttgttcattttctcaagaccaaggtgttctgttctttgccgggcctttccatagtgcttcggggagactgtgatttccaactctgacccagtgttgagggccatgtgtcacctatggtggcagtggttgactttctccattgttgggacattttctactgtgaatctactgtatcgttttagagctgcccctacctgctggagaaatgctagggcatctggagccttgcacttccataccctgagctgtcattaacccttcaggaggggagctggagccatgcctcctcagtggacctcgattgtgttcccagcactcccatgtttactcacaatcatctatgactccagttccaggagctcccatactcctagtgttgcaagcgctaggcacgcacagtgtgatcagtcatatatgtaggccttgtacatatagaataaaaataatctaattatggggtctcattacaggtgcttaaatctaaattactgcaagtgttcatattgggcctgtcatggtgaaaaaactggctcctttgactttaagccagcatttagtactggacctcttagacactctgtaaccccatggcttaggagatatggctttgatgaaggggcctgtcatcacagtatggagtcatcacagtatggagagactgaaataggatgagcaagaattcaaggctggacagactgggttatattatgagactgtctcaaaataaaacaaggacagaatgcttctgtcatacacatagccctgggttctattcccagcacctcaggaaccttacactgatggtgcacttgagattgctcaggaggtggaggcaggaggatcgggagttgaaggtctttctcagctgtgtgttaaattagaggtcatcctgggctctgggaaaccctgtccccagaaaacaaacagaaagtcaaaacaatacaccaccgaaatacaccatgaacacttctgcttgtttccatcactgctcctgaggctgctgctgctcttcctgtgcacacagcctctgggatccactgtggacactaccaggcttcacagcagagctgtgggaagacagtgttcaggccttacagagccagcttcagggactctgcacaacaccccgccctccccaacaacctcctaggtctgtgtatattgtcttggttatcactgaggagctaaagacacagtgaccagggagtgtctggccttgagccagcagaaacctttgccagcggaaccagagcccctctgccaggcagcctggcctgttgttaataggaactagggtagaaagctgcctgagtcagtacagtccatggagcgacttcagcctttgcaaactaggaccattagcatcctagcagacagggctgtaatgaggttggcagtggactatagcggcatctctgtctgccagatttgagaatgcagctgtctcaggtatgggcatgtacacacatgctgggcacctgcagaggtgattgggcttttagtgcgtgtggtactgagttccatctttttttttttttttttttttttaagatttattagccaagcatacaagcatagtggtgcacacctttgatcccagcactcaggagggcagaagcaggtggaccatatgtaggtatatggtctacagagcaagttccagtatagccagagctacacagagaagacccatatgggaatgaatattttgtctgcatgtaatgtgtgtgtgctacatgcatatcaggtgcttgtagaggccagaagagagcatctatcccttggaaccagagttcgagacaggtgggagccaccatgtgagaactgagaattgaactcaggccctctctccagccctcagccatactcttaattttacattagtatgtttgaaaacagagccacacataagccaagcatgttggtgcacatcattaatcccagcacttgggaggcagaggcaggcggatctctgagttcaaggatagccagggctacacagagaaatcctgtctcaaaaaaaaaggaggaaagaaaagaaagccacacatgggctgatgctggcttgaactcggagtgcagctgtgagagtatctgtataacatacacacatgttgtcacgtgggtgtgcgtgcatgtgaactgatgagatcccagaggcgagtctgacgtcagatgcgctgtgatttgaagctggctcaggagcagcgtggtggtaaaacactctcctaacatttataaatcctgactcccaacaatggcagtatcaataaaagagaaaaagaaagagaagagaatgaattccaatccacttccataacttaataacattaggccccaggtcacaggtcaggccgtggcccaaggaagacagcccacaaggcagtacccatgcccaccacattgagctaccaggcactgtccccttatcccagccttcctgtgtggctcttttccacctgcagatctccccttgtcctggtgatttagcctgcccgctacaccctgtcggggagagcaggggggggggggatgcaccagttatgtctgctgccctctgctgtgtgtgtgtgtgtgtactgtgccttccttgctgttctgtacactgtagtcacacacccctactttcctctagattcttcttctgattgacattgagcagtcgtacatcaacacaaaccatgaagacttcattggatttgccaagtacgtatacagccattgtcagaggggcagcaccaagaccagagtggtcatccatctgctggggatgctgggcaaagggtagagccatttgagagtgccttggagctttctgtgctgtgacttctgtcacagtgtcctcacctagcatgttgtctatggacatctcagggacaaggtgtgtactctgtggccattctcagggaagaggaggccagccaggttcatactctgcatgacttcttagagcttttcacagctggagcaccatgtgggctctgcctttcacacactgtacacagatacatacgcactgtagacttaaaaccagaaccttggggtgggatgtttccattgttagcacttggctagcatgcacaagacctggattagaccacagggccaaaacaacagataccacacctgattgtattggtcctgggtcagccataagctctggaccagctgggcaaacttgggtgccacggggggggggggggggggtgtgttctggtggctcatgcagattttcttagctttagacaataggataccaccaggcaccaaaagacagacccttgtgtccatctatactactgatgtactttcgtgccagatcccttgctggagcagtacacaatctaggccaggtccacctgtccacaggaagtccatgtgttaagacacagtccacttgccgggcggtggtggcgcacgcctttaatcccagcacttgggaggcagagacaggcagatttctgagttcaaggccatcctggtctacaaagtgagttccaggacagccaagactatacagagaaaccctgtctcgaaaaaccaaaaaaaaaaaaaaaaaaagacacagtccgctcaaggctcttggagtccagagggtggctatggggcttttagagctcgggttttagagacgtgctaagtgggagggcacaaggctgaactgtctattgtctctgggcagaactggtggaccatgagctctgagcgggcacatggtggatgaatgaagcgaatgactgactggtcgatcagaagccctccctgagcacgtgtagcctagggcccttagctgggtgatttcttccctggggtgggctgcattgggggctgggcaaggcagcctgtttaagccctgctcttccagccgtgcatgggcagcagagtagcccccattggttgctttcagtttcctcacctcaatgtgtctgcttctcacacttcatccacagttccttctcacaatctctcctgtttttcctttgaccactccacttctttctctggattccggggccttcccacttacccagctgtttctatactgaggagctggtcacagggtgggtattgcctgctgtgtgcctttttccctggtgtgtgacccggggcgtcctccctggggagcctcaaacctgtgggggatgttccagaagcccttgaattacaacagccattctctcaaccagggccccagccaggccttgtgaagaggactctgggtaagacaccatagagcctgccgtatagggcgcacagactggggagtcgtggacagtggcacagcagcctgagggatgagcactggggagaccgtcgcagagaaatcatgcagtggctgacaggggactgcagggagctggtactggacagggagatcaataggcaggacaccagccagtgaggcaggatggagacaacagctggtgaccacgcccctcatcagggagaccctgccatggcacccagtgtctctagttagggccatcaccatcttggctcctgtttgagggagctatgtagcacataatacataagtagcacatctggccccttgtcactatactgttgagtgatggcttagcttagtctcaccttcttcaggatggctcattatagacagggcctaaaggaaacaccctgtgatggctgctgccgtggcacagttggtagagcacttgcttagcatgcatgaagtctagcaccaaatcaaaggggtgtgatagcccactggaagagagagagagaaacagcacaattggtccaaagttatctttggttatatatctgaagtagaagccagtcaggaacacaggagaccctccctccctcaaaaaaaaaaaaaggccagtaagatgatttagcaggtaaaggtgcttgctgtctaagctgcatgactagttagatccctgtgtgcacaatgctgtgccctccaaaaaaaataaagataaaaagccgagccctgggagagtggtgtcagaagagcactaaatgggcatctgtgtgggtccttaccagtctagaataaagagcccatctcctccaaacactcactgcctttctagcaactgactgaagctttgttgctgctccctagtctctgtttaggctacttgaactcctgaggtagcccagcctaggcacagactcctcctaccttagcctccactggggcccctggcttgtacgctcacttaacaactttaaattgagtttcccaattgaaactcagctggtttgagctgtaggcagaatctcagagcagccagccggtggtgaaaccaagtgtgttgctatttgtgtccttctgcagaggacctgggttctgttcccagaacccacatcaggtagctcacaaccacctgctactccaggcttccagtgccctctggcctccatgcacacctcacatgtgtggtgcacataagctcatgcaggcaggcacacacagtgacatacatacacatgtacatctttttttttttttttttttttttaaagatttatttatttattatatgtaagtacactgtagctgtcttcagacactccagaagagggcgccagatcttgttagggatggttgtgagccaccatgtggttgctgggaattgaactcaggaccttcagaagaacagtcgggtgctcttatctgttgagccatctcaccagcccacacatgtacatctttataaaaaaaaaaaaagaaaagaaaactataacagatgagtgacaggacacggtttctttcagatggttatattactgttagaatatgcatcagaatcagtaaggagagtggtgggtagctacaggaagtgtagtgtggtctgtctacagcgccactgcatgcagagcacctggcatacacaaagctctgttttcgtccccagcacttcacatactgcatatggaggcagcctggagaacttgaagcccaacaatcccagcacttgggggggctgaggaaggcagatctctttcagtaactgtccagcctggtctacatgagatctaagatagccagggctacatagtgagaacctggagagacagagagagagagagagagagagagagagagagagagagagagagagagaggaagagagagaggaagagagagaggaagagagagaggaagagagagaggaagagggagctagagttgctgcgggtctgtgaaggggagcacacacgaggccttgtatttggtctgctgcattcccttctcaaagctgaaggtgcaagaacctcccatagtgggtttgggcaagtccttctagcagccatttggccagtgtaagccctagaactatcagggctggtgatgggagccacttgtagactgatgaagggtcagtggtgggaagagaatgtaaagaagccccaggtacgttgtgctcagggtcccctggaaccagcttcctgtcccagtcccctgcatcttacacctcttctttgaagctctaacttctagtgacttgtgggaaatagctggtgcttctggagtggccatgagaaggttctagaagccagggttagccagatacacttttctcatttcatgtgctctagttgttctctatgttcaggagccatggccttgcctctgcccttagtgggtcagtgggcccctagggaaggcacctcagcccgggccgccagggcatctctttacctccctcccatgtctcatctgaatatgccttctctgcttctgttacagtgcccagcagaggagcacgcagctgaacaagaagagggccatacccaatcaggtagctccccagtctctgctgtcactcccttctgaagacaggagcatacttagctcatcatagcatttgatatgggaaccgactccttagtactaggctgctctgtcagcttctgggtcatgtagctgttagaactaaggcttccacaggaccttggggcaaattgcaaacacccaaaggcctcatctgacatctaatgtgaaagttacaacttgaccctcaaagcaggagatgtgttacagtgaccccgaggtgtatgttttctaactcttggccccaggaatgaaggcaggtaggtagaaaccatggaaagtgccacaggcaactcagggaagatggggatggtttgaatgccacggctggagaaccccaggcctcctttataatcccaccccagcttcagaggcttcgcatacttacagatggcttcagagatgccagtattacgtgtctggttgggcttgtttttctgtccagtttctgcactgtcccccaattcccttcccatgtcagactggggtggggatggggcgctgtgctgttttatcttgcacgatttagagccagatggggagaaatgtaacaaatgcatgattttgagttttctttttgtatgtgtgcttgccataggtagtacatatgttgttgcttttttttgttttgatgaagataatgaattaaatattaacaccatgaatataattaatagcatgtaatttttttcttttgctgtttttttgttcctctgatcttccttctcctacccttgtgctttccctgcgttctctctgtccctacacgccccttccatctctcctgctctcccttgattgacccacaaccctaactttacattctcaaagggggagatcttggtaagtacctgcttagtgtggtagctaacttagagacctagcagtaaaggaggcctgccttcaccctctgctcaaaaaactaaaacgtgcactaacccgggagctacacgtcaccgctgtcactggacctcgcgctgtcctggggatctgttgctctcacaaaaatactaacccaattgcagctctctgggagacacgcccctctgctcagtcctagcaggttctcttggtgtcctgtagttcaaagatggcaaatatgcattcacacattgcctccggccccacctcagcttcctagacatgcagattttgctcaggacctgctggaattcatttctttttttttttttttttgtttttttggttttgtttttttttttggttttgtttttttgttttttttcgagacagggtttctctgtatagccctggctgtcctggacctcactttgtagaccatcctggcctcgaactcagaaatctgcctgcctctgctgggattaaaggcctgcgccaccacgcccagcttggaattcattcttgctgccaggccttatcctctttctcttcctgcagacatcactgactgaccagaccatggcttaccacatgaggagatttgcctttctgtgcctgtggcaggcatgactaaatgatccaacactctttcctgtgattttcctccacagtgaacccagggcagacactgctaatcaaccctggcattctttcttaatatgcacaccattgactccccattctgtagctattgtgactgattatttttttttccattttttattaggtatttaactcatttacatttccaatgctataccaaaagtcccccatatccacccaccgattattttttttttaattttccatatgatatcctactcctttactcacataggcataaataatcaggttccctctagcctgctgccttcatagcagctggatggtcagttccactttcccacttttcttatccattgcctgggacagatatcacagagtgatcccagcaccttttctgctgtgctcagaattgttgccatagactactcctaccagtcagttggtgtgcattgtatttgccatcctaactgtgggcaactcatcttccttttgctcaagggctgggagctggggttgtcagaagggtgtgaggcccagccctgctgaaagctgagtctgtgtggaggtgctccccgcaagcagcttggcctcaagccctgacagacccctttctccaggatattctcctgctgccaccaccattctctgcagggatattcttggagttttgacttttgcaaaggttgagccacttctgtgtgtaagtggtgggtccttgttgcacctgggtcctggcaaaagcaggctgggtcaaagtcgaaacctgggccagtgttagagaagaggggtctaggaccagattagcctcaggcctggagcaaagcagtcaagtagcacttctaggtggtcattgttcctaccttgttccattctcctggccacaaaagcacacacagcctggagatcctgagcgtggccggccgtagcctcgggtgccttggccatgcttggtcttccaagtgatcttcacccagagaggaaaccatcatggctcctgccgagctcgccttgctactacttgctgctcaagcccctgggatgggtcctttctgtcctgcatgcctgcatgccgttactgtgccccaggaatggccatcctagctgcctctgctcttgccacacctcccctagctccttctcacccagttccttacagccgtgaccctaggttccaaatctaccttggagataaggaggcacagccttcgctgttccctggaatcagtctgtctgtacttgtgggatcggtgttatctggggctacattggggtacccacatgggcggggggctggaaggaaagggtgctcacacaggcagggggcagagaaggaagggtgcccacacaggtggggggagtggggagcggggagggaagggcgtctacagaggcagggggtgggaggggcggggggcgctttggggattttgccagctcatgtatccttagtggaggcagctaggctagacctccaagggacagagtgacacagctgttacacagtagtacagagaccccactatgttggattaatcttcttggggtacagtggtagcttgggcctaagtttctctcccttggttgacatgtggtgactaagttcaataagctcttacgctgggctaagactgactgaaaggaccctgccacccatccctggcaggacagaacttggctacaggaccccaggctgggtgagttccaggattaagcagtcagcctacaagcctcaccagccagcctaggtggtctagaattgaggttcctggaatgctgaaaagcctcgtagctaagcccaggctgttttggaatgaatctgtataagtcagtaactctgtttctagcacgaacactatgccctcctggggctccaggcatctctaggagtctgagatctcttgacttcccagggagggagagattcttgccctcagctttgatgagggatgtcaagtggaagcttatgttgttcttggcccaagaagggacttaagtaggctggagggattcctgaacctttcatagctgctttggccttcatggcctgctgcttggaacccaaacacacccactgtagcagggtaggtgggcacaggccctgtttaggtttggcaactctagagccaaacaggacaggatagggaaaggggaacctcaactctagggccccagacaccctattttggattcttctgccccatcctacctgagaaacccaaacgcagcctccccacctgtccatagctatacatgtcacatgctctgcagcctggcccatctgttgctgccttggtcccaggcatccctgttgtcacctctgcatgcttcctgctgtccccagagtacccaggtcagggggtgggaggctcaggtctacatgtgcctccgtgtcccagggatcatgggaatctgaaaacgtggccccctcaggctgcctgtacctagcagtcaggtgagtccccaggctgacctggcctctgttccccatcagataacagctatggcccaacgagccctttccatggtgtgtctctcaaccgagacccctgagttgttactcatagaaaagtgtgcccactgggcttccagctagctgaaacacagtcccactctggtggacccttcagggtctctagtggtcctctggccagccctctgggtgggactgtctccctagctctggtcacctacacacagaagagcttgtgtgagtgagacacctttgtgggtcagtccatgaaagaggtcacagctgtcccctgctgtgggttctccagtgagtcatctctgcctaccttgtgcagatccccagtgtcacattctgcacagtctggccccagccctagccctggcctcctggcctaagccctttgttactcctaggacgtgcatttttgtttcctgtgtcatttacatcactgtaacccccaaggggcttctggggccctggcttgccattctgtccctgagctagagatgtgccctccgactgtctgcatccgtccccacagcggtcccggctccctgtctgattcctagctgggaatgcttggcttgacatggaactaggatcctggctggggtggaggtattgctgccacagagccaggcttctatccttactggggaggagggggtgggtggagcagcagggctgtcctccctgctaagctgaggcctctccaccaggtgatccgcaggggctggttgaccatcaacaatatcagcttgatgaagggtggctccaaggagtactggttcgtgctgacagctgagtcattgtcttggtacaaggatgaggaggtaagcagctggggacaagggtggcctgataaggtaggcaagggaggtggtccccagactgattaccccattcccccagtcacttgagaaagggagtttacagtcaggcatgctgacgtgctgagacccagcaccagtggtaaggtccaggcctgtccaagttatgtaggaagtgcccctgtcagacagtccagctggtaacatgcttatcctgccacgggagctctgactcatcccagaacctaagagaaaagggaggcgtgaagggcgctcgtattcccagcaggctccgcaggcagtgcctgctggccagccaggcttgcctccttgacaggctccaggcaaataagagacctgactcaaaaagccagagcgcagcgcctgagagatgacacctcaagtggttctctgtcttccagatccacttgcatgagtatgtgcctacacacaaacttgagtatacacccacacaaaagaagaaacaggaaagcaagccaggggccgtggtggtgtagcttagcatgcacggaacctttggtcccatccccacttagcatcgcatgcgtgcctgcgatgcccacactcagcaggctccttcactaacatagctcgttcaagggcagcctgcattacagtgaggttttatcaggaggacttccgtctgaaggagagaccctggcctcagttctctgctgctggatgggaactgagggggcatcctagtcagggccaggctcccaacatgtgctctggtggcctgtaccagcagtcacctctgtagggtgttccagccaccatgtaggagagacccacacacacaccagtgcaccaaagggacacaaggactcagtgtggtaccatatgtgacctgtccatgcacattccattcccatcttaggagctggccagcaccctggccctgaagtggtgtaagtgtcaccccaaggccagacttccctggtctagtttctgctgttgttggttttttggtttgttggtttgttttggttttggttttggttttttgagacagggtttctctgagtagccctggctgtcctggaacttactttgtagaccaggctggcctcaaactcagaaatcagcctgcctctgcctcccaagtgctgggattaaaggtatgcgccaccatgcctggctctgacctggccatattttttagcataacaaagactctgggattatcccccagaatgaggcacccagtgttgagtttccagctccagctgtaagaaaagtaacctgaaagctgggcagactctgtgtgtgtgtgtgtgtgtgtgtgtgtgtgtgtgtgttttcaaggttcattgcagaggagcgtagccctgggcctagctgacaagcttcttcctttggactgggaatgaccattccaattcccctcatctggagacagaaagcaggcacttgggcctcactgtagacaaggcctgtagcagggccacaccaccaccaagaggtatgaaaggagccctctgtatgtatgccaaagatgaagctcactgagatctaggggctgtggagaccctgacacctttaatggtcctggtaagggacctaagtatcccaggtagcagccatccctgagacccagaccactcttgtagacaagcccaagataaagaccagcaggggacttttctttattttaggagtaaaccagaagataacagtgtcgttggaaataaggagaaaggcccagttcacctgaggacatccctccccaccccagcgctcgctcagtcctgggcctactttttccaccctcctcccagaatcttccccagtggagagaggtagcactcaccttaggtggtctggaagttcccactgttgctgctggcccaagcccagcctaaccaatgtgcagactactgtacatgtcccagcctcctgaacaggtagtctgaacactgggatggcggggatggctgttcagttcctcagcgtggccatggtacaggccagggagtctgctcctcccaagatcctccattcagttcatccacgcaccctgaactcttctgacgtggcaaggccaaggccacttgctcacatctggagaaagttgagattttcaggaaatccgaaagcaggctggaggagggcttgggtcgatggcccgtgccgcagaaggaggtccttgtgcagttgcatactttgcgagcaagcgcatgcgtatgctcgtgtgtgtgtgtgtgtgtgtgtgtgtgtgtgtgtgtgtgtgtgtgtgtgcgtgcgtgtgagagaaagagagagagagagagatggcagccacccgatcctgggcagaatcacacataaacagttccagatgtggcaagcctagatgtctgtgactcatttacttggccctggcctgctggcaggaagctttctgtctcgtgacaccatagggtaggggccacagagttgcttctgggaacttaccctcctctggaaacagaggctcctttggggatccccaaaatcccaggtttgtttcttataacaagacagacttcaggccgccttccctgtctggagctctcggtatgcagtgtgtgtgtgtgtgtgtgtgtgtgcgcgcgcgcacgcgcgtgcgcgtgtgtgcgagtgttccacactccctgctcacagcctctccgcctgtgcttcattcaggcagtcagacctggtgaggttgactgggagccagtccatgaattggctggaaactggggccacaactgtgccaccctccccaagagtaacactcatcattcctcagacagatggggatcgggaacgcagaggagaagcagtgtgttcaaaggcagagcaaagcttgaacacagcagttcctcatacagtgttcagactggccccattctgcactagagctacgggggaattcagaattgtattccacaaagctgctttctgggactttctgttcgtgcagtggacagaaacatgagaaagagtcatgaaggatctccagtgagcctggagagagtgctctgtggggaagagtacttacgcacctgcgcacccatgggtgctgtgagtgcctgggacttcagcactaggtgtgcaaagaggaggaggctggggcttgctggctccaggtttagtaaaagcacttgtctcacagaagtaaacatcatcttccccatgaaggcctcttggttctcctcagtgtgttgcacatgacacacacaggaggacatgtccaggtgatgggctgaggcccagctcagttgttggagcttttcctagtgcttcatgaactgggtgtgactcatggctcaagcggtaactccttacagcattggtgagcaagatgcgagaggatcaggaattcagctcatcttcagctgaatagggaattgaatgctaccctgggctataggagactttattctggaaaaaaaaaaaaaagtctgtgtggtgcagaaaatccccagtgagctgttgtcatctacaaccattcttaggttagttacacagtgtccctgctctgtgctggacagacagcttcctaatctgagcagacagaaggaaaagtgccccccccccaaaaaaagctgggcagtggtggcacatgcctttaatcccagcacttgggaggcagaggcaggcagatttctgagttcgaggccagcctggtctacagagtgagttccaggacagtcggagtatacagagaaaccctgtctcagaaaaccaaaaatagaagaggaaggaaaagtgtcacaggcctattgttatggctccttgggaggcaggaggaacacaacttcatggaaggcctgtctcaaaagcaggaggaggaggagtgttgcctgcacggccagcatcgtgcttgcctgagagagaccctgggttccgtccccggcttgggtcgctagagcgcttgtcaggcatgcatggagtagcaaagccactgacgctcctgtgacagtggtgcccaggcatcagtttcccaggcctggcgtctgaaatagcctcctgcgcgccagtctccctccctgtgagtgattgccgccataactcagataatggggagagccacctgggaaggcatgaagttagggctgtgaggtctgagtgaccactgcatgtgtgttctctggatcaaaaacaaagctgacagccttcctttctggcaactgctggaccagccaggtggtaggcagtcatcatagcatctgtagtagggtggcatgaggtcagccctgagggaccttctgcatatagacctaggaaggccaggaggggggcagtacagggtgtagggacaagaacagtctggacggggtcggtccgtacacaggtcctgggatgagagagaatgttaagggaacgtttgaccaaaaaatcaaaaacagcacctgggaggcagaggcagacgaatttctgagttcgaggacagcctggtctacagagttgagttccagaatagccagggctatatagagaaaccctgtctcgaaaaaaacaaaaaacaaacaaaaaacaacaaggggacgtttgagtaaatgtggggtgacagagctctcagcacaccaagtgccgggtgttggcagtggctgtcagattgttgctggctctgccccggcagctcaggtgagacccctgccctctctcctcaggagaaagaaaagaagtacatgctgccactggacaacctcaagatccgggacgtggaaaagggcttcatgtccaacaagcacgtgtttgccatcttcaacacagagcagaggtgaggaagagccagcccagcaggccctttgccagggtcttgcctagcacagagctatcccagggacattcctgtgtggtgtgggcggagcaacatggacagacaactgacatggatgcagtaagcatgagggcctcagtgcaaccctcacacacatacaactctcatacacacaacaaccctcacacacaatcctcacacacacatagccctcacacaaacacacaaccctcacacacataatcctcacaaaaacatagccttcacacacacagtcttcacacacacacacacaaaaccctcacacgatacaatcctcacacacatacaaccttcacacacacaaccctcacatacacacaacactcacacacagtacaaggaaacgtctggcagggtgctggggaggcagaaagcaggatcctcggtgcccgctaaacctgctgaccggtctaggcaagtctgtgagcttcgggccagtgagacagaccctgtctaaaagagtgaggtgctcagataaagtgaggaagggcgtctggtgctgacctctgacctctgcatgcacatacacccatgcacttactcagggctcgacagtgcagacagtgtaggctgagtctattcctgctgctataggatattctggtagacattgttgctgtggtcttccttacaaacacctcagtgtctccacacctcagtgggcgatccaaagccagagctggactgtgtgcggtacttggtgctatagtatacctggcagacacacccagaaacctcttaacaaactagggaaggtactagaaccaccttcgtacccagatcaggcagagagcccccagagcccatggtcttagtggccagtcaagtccctctaccactcactccaggaatgtctacaaggaccttcgacagattgaactggcttgtgactcccaggaagatgtggacagctggaaggcttcgttcctgcgcgctggggtctacccagagaaggaccaggtgagggggttcctgcgcgctggggtctacccagagaaggaccaggtgaggggttcctgcgcgctggggtctacccagagaaggaccaggtgaggggttcctgtgtgctggggtctacccagagaaggaccaggtgaggggttcctgtgtgctggggtctacccagagaaggaaaaggtgagggggttcctgtgtgctggggtctacccagagaaggaaaaggtgaggggttcctgtgtgctggggtctacccagagaaggaccaggtgagggggttcctgcgcgctggggtctacccagagaaggaccaggtgaggggttcctgtgtgctggggtctacccagagaaggaccaggtgaggggttcctgtgtgctggggtctacccagagaaggaaaaggtgagggggttcctgtgtgctggggtctacccagagaaggaaaaggtgaggggttcctgtgtgctggggtctacccagagaaggaccaggtgagggggttcctgcacgctggggtctacccagagaaggaccaggtgaggggttcctgcgcgctggggtctacccagagaaggaccaggtgagggggttcctgtgtgctggggtctacccagagaaggaaaaggtgaggggttcctgcgcgctggggtctacccagagaaggaccaggtgaggggttcctgtgtgctggggtctacccagagaaggaaaaggtgaggggttcctgtgtgctggggtctacccagagaaggaaaaggtgaggggttcctgcgcgctggggtctacccagagaaggaccaggtgagggggttcctgtgtgctggggtctacccagagaaggaaaaggtgaggggttcctgcgcgctggggtctacccagagaaggaccaggtgaggggttcctgtgtgctggggtctacccagagaaggaaaaggtgaggggttcctgtgtgctggggtctacccagagaaggaaaaggtgaggggttcctatgtgctggggtctacccagagaaggaccaggtgagggggttcctgcgtgctggggtctacccagagaaggaaaaggtgagggggttcctgcgcgctggggtctacccagagaaggaccaggtgaggggttcctgtgtgctggggtctacccagagaaggaaaaggtgaggggttcctgtgtgctggggtctacccagagaaggaccaggtgagggggttcctgtgtgctggggtctacccagagaaggaaaaggtgaggggttcctgtgtgctggggtctacccagagaaggaaaaggtgaggggttcctgcgtgctggggtctacccagagaaggaccaggtgagggggttcctgtgtgctggggtctacccagagaaggaccaggtgaggggttcctgtgtgctggggtctacccagagaaggaccaggtgaagtgttcctgtatgctggggtctacccagagaaggaccaggtgagggggttcctgcgcgctggggtctacccagagaaggaaaaggtgaagggacaccttccctgctgaccctgcatggaggaagggacccgtgatttctaggccaagcgtgtcaagtcagcttttctttgctatggagatggcttaggtgatagcatgaggcctgaatgttagccctagaactcagtcaggagctatgtgtggtggtgcacactggggctcaggagccagccagccatagcacagcagagatctccaggtcctagtgagaggccccgactcaaaagtcaatctaaaagaacactaataacaagccagacacagcagatagcgtatgtgcctatagttgttgcagagtctggcaaagtggacacacttggggtgaaaaggggagtatggatccctaacgcccacttaccagacagcctagaaaaacctacaaactccagattcagtgagagtccctgtctcaaaatgtattgtgttttgtttttagggtgtgtctctgactagcctatagtttagtctgtagaccaggctggccttaaactcagatctatctgctcctgcctcctgaatgctacaaggcctgggatacaatgtaataacaggtcctggtatgttgatgcacacctttaatcccatttcaagaccagctttgactctatagtgagctctaggatagccaggactatgtagaacctgtcattcattcatagacagacagacagacagacagacagacagacagacagacagagtaaacataaaagttctgaggcactgagttgggatagactacatgcacacaatgtgtaagggtgatatgcacataaggtacatacatgaaacacaagtgaataaatacctgctacacaccaagggaaagaaatgttcagtttggctcccaatgttagagggctcagtccatggctgcatggtcccatgtgtataggctgtctcatggcagtggtattgaggtgcggaagcgagaagcagaggggaaggcatgaaagggctggggccaaggtatccctggggatacacacctagtggcctacttcccccagcctgaagttttcagagcttcacaagtaacagtagtatggttctaaaaggacggctttgcagacagaagccatggctactcctcaaaggatccaagttcagtccccaccacccacatgacagttgacagctgtctgtggcctctggacacctgcacacacacactgcatatacacataccataccaatgtacacataaaaatcagaagtacagtactgtcagctgagactcaagggaccaacacctgagccctagggacatcccatattcaaaccatagcagcatacttcagacctcagagggcaccaagctggtacccactgagtcaagcgaagcggaggaggcatgggttgtcatcagggtccagaagttctccagaaggggttctccatggtgaggacggcaagagcacttagggttcatccaccttgttgtcttttccctcttgtccttggggtgaagtcactacagaccctggccatccaaagggaggctgtgttctcatcccccggctctaaccagtgttaccccagtgcagactacaaggtctaccagggccatctggaacacactcaggtggggcatttgtcagcagggtaggcccagcacagtgaaccatccccctctgtgtcctctgcaggcagagaatgaggatggagcacaagagaacaccttctccatggacccgcagctggagcgacaggtggagaccatccgtaacctggtggactcctacgtggccatcatcaacaagtccatccgtgaccttatgccaaagaccatcatgcacctcatgatcaacaacgtgagtgacagcttccaagtcaccaaagatcctagccgcatgccagtcagcccagcaccagcaggtggaagcaggagagtcacaagtccaccatcctctcagatactcagccggtgtagggggctttcactgtgagaccttgtctcagcaatgacaactggggaagacggggggaggggtggggggtcagaaggtaaaggcgcgcgtgcacgtgcacacacacacacagtcaatacatactgttaacttaaaagcaaacctaattttaaaaagctgtgcaaagttagaacaagttggtttgtttagtagctttttggggttttggtgtgttggttggttgggtgggtgggtgggtgggtggttggtgtgtaggttggttggttggtgtgttggtgggctgcttggtgtgttggtgggctgcttggtgtgttggtgggttgcttggtgtgttggtgggttgcttggtgtgttggtgggttgcttggttggtttggttggttggtttttgtggtattggagattgaacccaaggcatggtgattcttaggcaagcactgaatgactgagcacctcagctccttccctgggagattctaggcagaagctttacagctaaacttgactctcactcagtctgtgatggatgctttcccaatctcaacaggcttctgagttattggctaatataggggtctgacctttccttcccaccactgtcctcagacaaaggccttcatccaccatgagctgctggcttacctgtattcatcagcagaccagagcagcctcatggaagagtcagccgagcaggctcagcggcgggacgacatgctgcgcatgtaccacgcactgaaggaggccctcaacattattggggacatcagcaccagcaccgtgtccacacccgtgcccccaccggtcgatgacacgtggctccaaaacaccagcggccacaggtctggacagcagtttgtcccccaatgtgcatatacaagttcaagccccacccttcctattccttctgtcccattaggaaatagttcacagctgaagtttggaaagggacaagagtcttaagtgaatggcacaacacctgttctgcttaaagccacaggggagccatggcttccaggtgccaacctgagctagatctctggaggagaaaaccgcacaccccaattctccaccaactcttgcatcttctagtgctttctccgtgggtccaagtagaaagcaaaagcaagaagcccaggagacagttacccgaggttggcctcttagaacatagagacggtacagaacacgttttagaaggcctgtagaacctagaactccattggcctcgagatgggaaagatgggccagagacactacggtagtcacagtccaagtgctaaagtggcagtcaagggaatgtcagtacaggggacccactacggaccctgtggagccctcaggaaaaggaagagcccaaacctggcaagattcagtccctaggtccagtggggagcagggcaggcagatatgtagaggcagaggtgatacatcaggactggggctgggctctaagtggatcagaaggtcaagtgcagtctgcacacctgtcccttggaggtttactgtcaggtacacaggagcctcagcaacatggaatggtgtggctcaggctggtgggtgcctctacccccacctcagtcacttgtggtagatgttcacaatatccttcctccagattacagcctttccttcctccatagccccactccacagcgccgacctgtgtccagtgtgcacccaccaggccggccacctgcagtgcggggtcctacaccggggcctcccctgattcctatgcctgtgggggccacatcctccttctcagcacctcccatcccatcccggcctggaccacagagtgtgtttgccaacaacgaccccttctctgcgccacctcagataccatctcggccagcacggattccccctggtatccctccaggagtgcccaggtaaggcccacgacctgcatcctaggctccctttgtggtatctttcacctttccccccttctgatggcttccagtgtagccccaacaggacatagaacagcacaggcatccctgcttgtgtcctgagacattgagccctaaaggggccaacccctgctcagccagtgccacagagcagtggaccagctgatgccagggttcctgcctgtcctctaagcagcaggtgacatgtctcactagccctgggcaccataccactgttgaccctgctgaagtcagcaatgcctggtgagccaggttctggtctccacttctgagatcagtgaggcaggcaagatggcacaagtgcttgctaccaagcctgatgacttgagttcagtctctaggaccctctgatggagggagaggactagctcttcccaaggctgagaacttgcctataaaacccaggcaaactagtgacatggggatactaggaaagtatctggacttgacatagtcccttgtacccccaatctgggagcccctcgggtgggctggaagggctgggtccccatcctcaccctgcttctgccctcactaccctccacctccccgtatctttactttctttgcagcagaagagcgcccgctgcgcccagccggcccaccattatccgcccagccgagccgtccctgctcgactaggctgcagggggcaatgttctgggggggtcctcgtgcacccacagtgtaggacagttttggtggtctgggccctgctacttgccctccatgctgggaccaggctccccgtgggcaagccctgccctttccctcctcgccctagtggacatgacaatgaagggtgaggaggcccacagcacagcacatgggggctttgcactttgggatgaggaggagctctggctagcagaacaggaactgcatccttgggaccatcaggaaaaaaaggtccaggctagtgtggggggactcttctggagctcctccagagcctttgttctttctggggtccaggaactgcccacctttcctaaggactcctcaggagtgagccaggcccagcaggtccagtgctgacccacctggctcaagttgtatatatagagctcctttggccatattaaccacacaagctgagcccagcccaggctccgcacccagaggtgcctttgtgccttcctcaggcctggagggcctcagctctggccaccctcactctcctcagccccggttgtggtttgggctgtatgaactggggagacatcagaggcctcttggctcccaaccagatgtccccacagggtgggccctgggtgcctctgcagcctcggctcccaccgcctggattgaccactgttaagtgcctgcctctgtatatcctattaataaactaaaataaagggaagccctactggtggctacgtgtgtgggccttttgtgtgtgtcccagctcaggagattgagcaccccctcatgaggctgcccacactcatttacacgcgtgagtcttgtgtcatctgttctgagcccctcttgtccccatccgtaccaggcctcacacacagtgctcatgggaaacaggaattctatccacctctgtgcgatgctgtggccaagccctcctcccagccccgccagccaccgggaccaggggactcagttcattcagccatggtttatggtctttttctttcgtgccaggcgaccacctccattggctcctgcccgacctttcttctgagcttcgtgggagtgtccccccttcctggctgccctggcttgaggctaggtctgtcccaagctgacatccctaagcccagtcaccaggcactattaactttctgaccataatttattgactccagtgcccaggacaaatgccctctgttccttgaggctcatgggccagccttctctgaggccctgcacccagagagaaggaaggggcctctcccaaaccacctactagctggctcccctctgggccaaggggccagggctccttcctttcaaataaaagtgactgtcctggcaagaactctgcagatgtgtctccttcccagaccctagtgaagtaggcttaccccaggaggagagcagagacattgcagttggggtgtctagttggagggtggggggcagcaccacagcttcaccccatcctgatagctgcaagactgaagctaaggcacgatttcaggagaaagccagggcatccaagagcagtgtccccacactgctgcgcccagcacttccaccctccaaggaacaggcacagtcgctgagccctcccccttccgaggggtctgtgagccattggcactccctggtcactgaggaagggagacacatggggccactgctaaggacccaactatgtagtgtctcatctgagtctgggaggtgtccagcaagtcacacacccgctgccctcatcctctgtggcaggggctacgtgggtacagggcgcttgtcatggaagaagcgcttgagtgtgcagacttgcaggacagccaccagcagcaacacagccacattggcagctgaccagaagttgacccgctccaggttgtcttcttgaagatttcgatcacgagcctcaaaggctcggagcagagtgagcatctggatgctccgttccagccgggtcctcatggtctctatggattcctgccagggcacaaaggaacgggtcaggctgcttaagtcagaatgcctacctaccccaccacgcacggctctggctatccgttcacctgcctcacgtcttctctagaaactgaggggttttccccttggtgtttttttttttgttttttgggtgggtgtgagacttttctttttaagagggattggggcacacgtgcatggcagacgaccccaggtagccaggcttggtgcagttgcctttgcctatcaagccacttcaccaaactctgctagtctgtgcaagccaaggatgaccttgaacttgtgatcctcaggaccaagagaaccatacccagtgtgtgcagtgctgggtgtgggggctcagctgtgggagcgtcctaggtaagtgctctgccaactcaattattcccggccctttctgttggttttgtgagacgggagcttgctatgtaaataaggttggtcttgaactcactggcatcctgcctcagcctcctcagaactgggattctaagcatgcaccatcatacccagctcaccttgatgtcttccattttgacatcaagcatctcttctggctccacagcctccgcccaaccttctacctcctcctcatcttggaagctgtcaaagatgagctcaaagaacacaagcttttctgagatggtgctgaaggagttgtcaaagcacagcctgtagtctccggcctcagtaggctccaccctgggcagacagatgagacatagacgtgaccctggagtgaccagccagctggtctgtggaaccagtaggatggtaacctgaggccactggggagctgcagggctgtaactgaaccaaggcagggcaggacttcactcacgtgtgtaccccatcagcctttcgagactcactgaccaacagcacaccctgagggctctccaaggtgaagtccacgtccagcccagcacctccgatcacctgaagagcaggtaagaattggggaactggcagctttgtgcaagcctatctgccacagaaagcctgggttaaaccgaaccgggaaagacccatgtaagatcaccaagaacctatctttaagaagaaaaaagaacaaccacaaatcccaggtatttgcttgagatagaggagtagcgcctgtggagggagattagtcagagctgcacttctaccatagagttaggaaactcgggtcgtcgtagtttagcagcaagcggtttttgttttgttttgttttgttttaacccagcaagctatttggattcccccaagggcctatctgaagaccaacatccatccacacctacaaggtttcttcccagtccctaagatggatacatccacgtgaggtcttttatcaaccaccctgccaacgcccttgagtagtctaaggtggctgtctacctgaccgctaacttaatcgtaacctacccaacagccgccaggaacttccctagaacccaacccaaagagctctgagcagagcatgccccttctctgcaagactattagcgttctggccacacctctgccccgcccctcccgcctgttggccacgcccccagccctgctctattccgagcccgggaccccctctctcacctggtactcggtctcaagactagcattggccggtgcggactgatagaaacactgctttctcccggcgggaagcagaaatgtgaactcgccgtcctggataggcggcggccctgcctctcccactcctactgctggcaggagtagccacagggccagtgctacggccgcgccggccgccatcatccgggtcaccctctactcacggggcgtggaccctttaaggatttgtcctgcccacctcttatttcggcaaagctcattggcttcggcttctgttcgaccccaaagccgcggaatgctgactgaagggccccaggatggggagctgccctggtaattgcctaatctgctagaaagcatactaacgctactgcgaacttcgtgaaacctacccaagtctgatatcaaccactgcatttggtgggggcactgacaaatctaaaaatggtgaagcacttctttacaggcaacaattaagatgactaaacggggaggtttgcaggtcctcccttggcaccgcccgctctctgccttccccaattcaaggtgcttttgcccattggtcaatcaagtgtaggcgtagccctccgaggacggcggctttgttggattctgatctcaacttgtgtgacgggtgcgacaggcagatatgccaggtgctagtattccaatattggagttctggcgatgttagttcagtgagcatggcctcccccgactgcttttcctttacaccttataccatccctaagggggtggtggaatgtttgttggtgagtagaatactgcctcagcctgcgtacaatacatctcaaacctttgggtcagcaggtccacgtgcatctgtgctgacacatgcagtattccagtacttgaaaggctgagtcgggaggtggaaggagttcgaggccatcctctcgaactggtatggaactacatagttccagaccagcaaaaaaatcaaggaatctattttgggctttgaatggctcagagaacacaggcgc

SEQ ID NO: 48 - DNA, 16 bp, synthetic construct
  source 1..16: mol_type = other DNA
  modified_base 5: mod_base = OTHER; note = uracil
agactctcggttccga

SEQ ID NO: 49 - DNA, 16 bp, synthetic construct
  source 1..16: mol_type = other DNA
agactctcggttccga

SEQ ID NO: 50 - DNA, 18 bp, synthetic construct
  source 1..18: mol_type = other DNA
agaggagaccgagcgaat

SEQ ID NO: 51 - DNA, 23 bp, synthetic construct
  source 1..23: mol_type = other DNA
catggtttgtgttgatgtacgac

SEQ ID NO: 52 - DNA, 24 bp, synthetic construct
  source 1..24: mol_type = other DNA
cctacatcagggagcgagaaggga

SEQ ID NO: 53 - DNA, 16 bp, synthetic construct
  source 1..16: mol_type = other DNA
agcactttattgagtt

SEQ ID NO: 54 - DNA, 15000 bp, Mus musculus
  source 1..15000: mol_type = genomic DNA
tctcaggggaaaggtatcctggattcagtaaccaaggaacatcacaatgtcacattgcacaacaaacctcacacaagagatttattgggagagatacaagaaggtggctgcctttccttggtggagaaacagcaggaaacggagtgggaggcaagctttcttggtggtggagttttccagggtggaaatttctagggaggggattggtaaggtttggtgagttttcaaaccctgaggtgtgtgcttggaccttacaccctacacatgcacctcatgatacaacgcccacttcccatacggagccctgtgctcaccacagtccttcttggactggctgaatttaggctcctgcataaccttgtagcccttccctcctttcctactgcaaaaccctcttagcttgctgtggtaaatacacttctgtaattcccgtaattaggaggtggaggcagggaaatagcaaagttaggtcactctagatcaagtccctcttgcaaaaacaaagaaaaaaaccacgccctctatggtacaaaccagttcctcttccctagagttctggcccctccccgggaggatgtggtcatagggtcaggtcctggggccaaggtctgtcctggtacaaccccggactgaccggtttctttcactttggtcggctctgctatgtggctcggccttgcttttgtgatggctaacgttatgatggcagttaattttatgtttaaaagtttaaaatgtctgtgcttaagaaaactataaagcgttaagagtggttaaggcttagaatttgtttttacaactattttggtttctataaatgagcttgttttgctccagaggaccctctgcttggcccagaacaaattgtaactttgtattccttgcctttataaacccttgactgaggtagctgggtgctgcattagggaatcccaacttgcaaatacggacctggatggagccagtatctgaataaaagcctcttctttgttagaatgtattaatggtcagactggcgagtctctgaatgaccccagacccataacactgtcttcacgtcctagtggcaatcaccaaaagtcccttttgaaaccaaaagttgaataagtgtcccgttgttcctccggtcttgcaaggccttgcaaagctgatcacagccttcctctcttcctggagcaccaggaacctgttatagtctgcctttattgtctataaaatgaaatcacctatgaagattccccaccctctcttccctggttgcttggctttttcttctcagactgtcccagaccagtgatagatgcccaccaacccggatcagtggtagatgcccactaacaatcccagaccctagtaagggagggtaagggggtgggggtgggggttggggctggtggcacttgcctttaatccctgcattcaggaggcagacgaaagcttatttctcagttgtagtctagagatgccatgctctggctcttgaactaagaaggtaccactggcaactctggaagcccaggagggagggagggagggagggaggaagggagggagggagggaggaagggagggagggagggagggagggagggagggagggagggagggagggttggttcctactgagcaaaataaatgctgggtagaaaaagccacagtgtcctgctccccaccttctgcccagcacttcagctgtaagacaaggcaatgccctttgatttagcacttcactctggaaagagtacaattctgagatgacagcgagatcatcatccctgtgtatttctgcctgctcccagtgacccaagttgaccgtgattgaggttattttgtaaaatatttccttgaggcaccgggaatcaaggccatgcatcctaggcgagcattctgccactgagtctcatgcttccaccgaggagccacatccccagtccttttgtaagagaatttgccgatgtaggtggccatggtggcacaggaaattagggaggctgaagcaggcaggagaatcacaattaagtcaacctgagaaacatagtgtctccagaggaaaagaatttgcaactgtgattaagggcccatattgagtctggctacacagccctgactaaccagaactcactgtgtagacagctggctggcctcgaactcatacagacctacctgcctttgcatcatggcagtaaaatcatgtaccgccatggactctcactctgtgggagtatccttgatgggcctgacccaatcggatgagccctgaaaagggaccagaagtttcctggcaaagcagacacagtgtgaagcaattcaatgcagggacattctctgctccaccctctgttttattgcttttaattcttaaaattaatttatttattctatgtacaatgttttccctcatgcatgcccatgcaccatgtgcatgcctgatacctgcagagtccagaagaggcaatcagaacccctggaattggagttatagacagctctgagccctcctgtaggtagatgggaatagagcctggatcctttggatctaaagtccatacctgctgagcgacacctccagccctgacactggctttaaagttagaggaagtaggtggccagaaatgtagaaatcacacgtagccgtggtcgacagctgagaagaagctgggacatctgtcctgcagccacatggaagtgagttctgaccaggacctggggggggatctcactttagaggctccagagacttcagcttgaacctcagcgttctgatgatctgggcagaaactctaactatgcagcaccaagtgtttacccatgggtctgtgcatccgtaaaagtagttttggggggcattgttttagggtgctcagagtgtggtaattggtcacacagcagaaggaagctgcatgaaggaaggtcacataagcattctgtaagattctgaaagcaggttttgtcccatcattcttaccttaccctcaacaggacctggaacaccatcctagtaaagtcaggaaaatggagggcaggaacctaactcttcagtccgctactcccaccagtgactccctgctcaaaacacttctacagcgctgttctgctgggtctcccatgaagcccttgctcctcagacccaggagcaagcttgaccaatctctacctctgaagcttctgagacctttgcccgcatctattgatcctcactcctgggcaggcagcggggccatcggcagacgccatgacggctctgttgttcctggggtctctgctgatgagtctggatctgacactttcggtgaggacggtggacactgggattgagccagagtggcagtgaggggtccctgttccggatttagcctctgcctgtcacctgctagttcaggtgactgagagacttgtggtgggggtgggggttgggggaaggcacagctagcccagaagatccaaacaaaggaggtagcagcccatgctggtggatgtgaggacacaggctacttttgtgggggagtcaatgagaagagaaaggaaggctacactttgtgaggaaatcctgtactttggcgaggtggcaggaggttgaggcctataggatcaagggaagtctaacattgtgggcaatgttcctgccttctctctcctataggctccaccatggaaagactccaagaaatttaaggacgcacctgatgggcccacagtgggtaagtgatctggcctcttttcctaatctactctgtggggatggctgtgctttagcctgcgtggtctcaggcctctggagcaaggagggatcctgagccaagctgttccgttgacatttaaacctgggtgcctacagattcagatgtgtgcccggagtgccaggcttgggctgtcctggatgcctgtgttagagcctggcaggtactctgtagtgttggagatgatgtcatctgatgatgtcatcacacctgcacaccaacaagtgctcgccagtttccccatcttcctccctttcctttcaacactcctggagaatgtcagacataaccggtagagttcagagcttcagtgggcgatgggagccagaacagagctttagactcacccaagatcacagacaccccaagttgtctccaaaccctaactttctcacttgtgagtttactctctgcttcatttttccctgtctgaaaagttagacgggttgctgaggagaaagggttcctcattgttgtagatgctgctgccaggaaaatgccctaacatgttagctcttaggctagggcatagctcactggcagagcacttgcccaacatgcacgcacacacacctgggtttggtctgcagcactgtgaggggaaaaaattccttttttttttaattttaaagaaaaaattctttcttaagctggagagataactcagaggttaagagcactggctgctcttccagaggtcctgagttcaattcccaacaaccacatggtggctcacaaccatctatctgtactgggatctgatgcctttttctggtctgtctgaagacagcaacacatatagtaaataattaattaattaaagaaagaacaattcttttttaaagatttatttatttattttatgtatgagtgttctatctgcatgtacacctgtatgccagaagagggtatcagatctcattacagatagctgtgagccaccatgtggttgctgggagttgaactcaggacctctacaagaacagacagtagatctcaaatgctgagccatttttccagcccaagaaaaaaaatcttaacttttagtaaatttgacttaggtgaagggttccacccccatcctgccccaaaccctgctataggacttgtgagtacagaactctaccacagtgagccttgtctgtggggtcaagctgaggcttgtagtgtgatgtctctcactgaagcccaactcagtgtgaccctccaggcttctgtcctaaccactctaagccaatcagctgctgtccctttcaacctgacccaccatggccccctgtctccaagttctaggacccttccctctctctatcaccctgttgctaaattgccactaaaaggagaaaagaagaagaagaagaagaagaagaagaagaagaagaagaagaagaagaagaagaagaggaagaaacaaaacacatttgttgagcacctactgagtcctggactcaatgtcccaagcattgccactaagcctcacggtgttcccctgaagtagacagtgatttctttttcattttctctttgagattataatataattacatcactgggcagtggtggcacatgcctttaatcctagcacttgggaggcagaggcaggcagatttctgagttcgaggccagcctgctctacacagtgagttccaggacagccagggctacacagagaaaccctgtctcgaaaacaaaaacaatatatatatatatatatatatatatatatatatacacatatatatgtgtgtgtgtgtgtgtatacgtatatatatatatacacatatatatatacacatatatatatacatatatataatcatatcatccccctccctttcttccctccaaaccctcccatagccccaataattatttctttttttttttaagatttatttatttattatatgtaagtacactgtagctgtcctcagatactccagaagagggcatcagattttgttacggatggttgtgagccaccatgtggttgctgggatttgaactcgggaccttcggaagagcagtcggcgctcttaaccactgagccatctcgccagccccaataattatttcttatgattaactatacttttctcagctaccatgaacatcaagttcacaaattcagtgattccgttcacaaattaggttccgtgagcctctggctatgacattttcaccaatcattagcatgtaatcttgttacatctggattctgtttaaagacattattcagtgtgcatgttgttgatcccttaaacactaatctaatggccgatgccactattactgcctgcctgacaaggtgtccctgacacgttttcttcataaggcgcatacaacttcttgtgttcttgcaagtaggagcctcaggtaacacttgggcatacacatagaaccactctgtctccctgaagtactgaggatcgaacctagagccttgtgtatgccaggcaaatgcttggccactgagttaaaactctggccctctttaaaaagaagtgtgtgtgtatgtgtgtgtgcaagtgcatgtgtgtatgtgtatgtacatatgtgtgtgtgtttgtgtgtgtgtatgtgtgtatatgtgtgcgtgtgtgtgtgcaagtacatgtgtgtgtatgtgtgtgtgttcgagcatctttcactgacccattcaattccataaagatcatgaaggcactgtgaacgttggcagtttctttggctttctttggccttgtgctgacttgtgagcaagcactacacgactgacctatatcctcagcctgagtcacaagtattgggttggagggggcttacaaataaatgtttacaaagcaggcaggggcagtggtgggggggtgcctttaatcccagcacttggcaggcagaggcaggtgaatttctgagtttgaggccagcctggtctacacagtgagttccaggacagccagggctacacaaagaaaccctgtcttgggggtgggggtgggggaacaaaacaaaaaacaaaacaaaacaaagcaggcaggttcacaaatataaagtcagtgaggattggcagttgcattactgttgccagtttactaagccaaaaatggccaagggacctaagtgtttgtttgtttgtttgtttgtttgtttgtttgtttgtttgttttgtagacaggctatctctgtgtaatagacctggctgtcctggaactcattctgtagaccaggctggccttgaactcacagagatccgcctgcctcagcctcccaaattctgggatgaaaggcgtgtgacctaaatctttaatgagctcaactttgtgacatgcacctgtgtttcagacttgctcagggtcagatatctattccagctgtctgactctagaagcctactttctaagcaggatgcagtcttgaatgcatagctgacctttttccaaatcaggccctgtgaggcagagggtgggcagctgggtctcagggcagggctctgaggtgtggtgttcctacagttctcactgtggatgggaggctctgccattttccctttcagtaccaccgtcagctacaccacaaatgcatccacaaaaggcggccaggctcccgcccctggtaagacattttctaataggggttgggggagggggctggatgaaatggactctgtctagctatctaagcatcgtgtttggtcaaaaggtgtgtgtgtgtgtgtgtgtgtgtgtgtgtgtgtgtttgtgtgtgtgtgtgaagcaaggctagcctagccagcctcaggcactgcagggaaacctcatctatcccccaggtgtgctaccacccccaactttgatgaagatcagcaatggggatactgcttggagcccaagaaagtgaaaggtatgtatggcatgcaggccccgggtggctcaaggctgtgtgtgtcttgctgctcactccgttcccacacacctactcacctactaagcatcatcagatgccaaacacttgggattctgggccccgcccttttccttctcttacagcattctttcaaaagtcagaggaagaattctggggaaagaggccaccattgtccagatgagcttgagggtctagaagcttggggttctatggtttgatgacaagcaggtgcaggcagggtattggtccctacatatgtccttccgactccggaagtgcttcgctcttgttatctacctctcagaccattgcagcaaacacaacccgtgccacaaaggagggacatgtatcaacacccccaatgggccacactgtctctgccctgaacacctcactgggaaacattgccagaaaggtaagaggaactgcctcccagcaagatgtccctggagacccggtgctttgccatggtcccattgacttccttgtgtccccagagaaatgctttgagcctcagcttctcaagttcttccacgagaatgagctatggtttagaacggggccaggaggtgtggccaggtgcgagtgcaaaggttctgaggctcactgcaagccggtggccagccagggtaagtgggtgtgcagggactgtggggaggagggcagagagtcaggaacccctggtagaaggctgggtgcaatgatgtacacaggtaaggctcagtttgcacctctccccaccccacccccagcctgcagcatcaatccgtgccttaatgggggcagctgcctcctcgtggaggaccacccactgtgccgttgccctacaggctacactggatatttttgcgacttgggtgagtaagaccccgtgtggaaaggcttgcggaggtggatagagagaatggaagtgaaccagagggctccaacagactcatccgccgactgcagggagccatctctctttctctagacctttgggcgacctgctatgaaggcagggggctcagctaccggggccaggctggaactacgcaatcgggtgcgccatgtcagcggtggaccgtggaggccacctaccggaacatgactgagaagcaagcgctaagctggggcctgggccaccacgcattttgccggttcgcgagaagggaccgggcaggggaacttgctttctcttagggtcctcgagggcctccccacgttctaacagtgctccctcttgagattgcaggaacccagataatgacacacgtccatggtgcttcgtctggagtggcgacaggctgagctgggactattgcggcctggagcagtgccagacgccaacgtttgcacctctagttgtccctgagagtcaggaggagtccccgtcccaggcaccatctctgtcccatgcaccaaatggttaggcagaggagggggtcccggcgcagaggacatgggtctctcttattcctggcagcccgtgccaggtatccatggcctcagccagtctctccttccacagactcgaccgatcatcagacttctctgtccaagaccaacacgatgggctgcggacagaggttccgcaagggactgtcctcgttcatgcgcgtggtgggcggactagtggctctgcctgggtcgcacccctacatcgctgcactgtactggggtaacaacttctgcgcgggcagtctcatcgccccctgttgggtgctgaccgcggctcactgcctgcagaatcggcaagtgccaccctcggtgaccccctagaccgctcctaccgtacccgcaccctactctttccctgcccgccattcttgagctccctcgaggggttggaaactaaggcacccccagagcatttgtagccggtctgagcctgctgcctgtcccccacccgactgcaggccagcgcccgaggaactgacagtggtacttggtcaagatcgccacaaccagagctgcgagtggtgccagactctggctgtgcgctcctaccgccttcacgagggcttctcctccatcacctaccagcacgacttgggtggggtggccctacagggatagggagaaaggatggcggagggctggggccctatgtcgccatctaacctttgcctctcggggtagctctgctgcgcctgcaggaaagcaaaaccaacagttgcgcgatcctgtcacctcacgttcagcctgtgtgtctacccagcggcgcggccccaccctctgagacagtgctctgcgaggtggccggctggggtcaccagttcgagggtaggcacaactgttgggcgctggttggagacttttggttatctaggagcgcagttggtacgccccgatgaatctgggggacaagtttcactgacatgacagttgtaaaaaacgcacagagcccttgtctctgtagcgtgactttcccagattctagaattctctgtcgagattccagagccccttgaggtttgttctagttttttgccttctattgtggcgttaaacaccatgaccaaatccagcttaggcaggaaaggttttatttggcttccgaggttgcagggtttcttgctgtccaccattgaggaagccacaacagggagtggaggcaggaactgaggcaggaaacggaaagggacactccttgctgcctttctcttcatggcttgttcagctttctttttaaaaaattcacttatttttattttatgtgcatgtgagtctgcctacaagcatatgtgtgcaccatatgtacatctggtacaggggaagccagatctgtaatacaggttaaaaaccacggtgtgggggctgggaacggaaccaaggccctctgcaagaacagcaagagctcttaacctctgagctagcccccaacaccttgcggcttgcttactttgctttgcttttctcttttcctcttcctcttcctcttcctcttcctcctcctcctctttctccccctcttcctcttgttttgtttttgttttttgttttgaaggcagggtttctctgtgtagtcccagctgtccgggagctgtccaggagctgtccagctctgtagaccaggctgccttcaaccttaagcactccatctgcctttgcctcccaagtgctgggattaaaggcttgcacctttctggctcagtttctttcttacacaatccaggcccacctgttcagggcggcatcactcacagtgggcggggccctttcacatcaaccattaatcaagaaaatgcccggggctggtgagatggctcagtgggtaagagcacccgactgctcttccgaaggtccagagttcaaatcccagcaaccacatggtggctcacaaccatctgtaacgagatctggcgccttcttctggagtgtctgaagacagctacagtgtacttacatataattaataaataaatctttaaaaaaaaaaaaaaagaaaatgccctacagatattgccctaggccaatcttctggaggcatctcttcgggtgaggttccttccccccctagatgactcttgagtgcgtcaaattgacagacagtaacccagcacaaggattacagggagacttgaactgtcttgtgcttcttttgggctccaaattataaaggcttaaccaggactttgtccccatgctggagcaatggagacaatggagagaactattctgggagccaggttctgagccactgtgtggaagaggataggaagtgcttcctgtgttttaagccctgctcttctctgggcttcagtgtccttgccatgaaatacttattggcaggtccctatcactcaggcttgctgtgagggagcaaagcggagtaggtggggaattgtctggtagcctggcccacgcagcaagctcaggtcctcccctctgatttgcaggggctgaagaatactccaccttcctgcaggaggcacaggttccctttatcgccctggatcgctgctccaactctaacgtgcacggagacgccattctccctgggatgctttgcgctggcttcttggagggaggcaccgatgcctgccaggtcagccctggggtcctggtaggtaccttggtccctgcctgtcaagcataaggcaagaaccacgtgctgcctgttccccacccagggtgactccgggggccctctggtgtgtgaggaaggaactgcagaacatcagctcaccctgcgcggagtcatcagctggggctccggctgtggtgaccgcaacaagcccggagtctacacagacgtggccaactacctggcttggatccagaagcatattgcttcataactaaccaggctttatccttccctccttgtgtgctccttgggatgggacgatgaatgtggcatgctgggtcacagtgaagctagtgccccgacactgggggcacagaaactcaataaagtgctttgaaaacgttcctcagaattctgtcttgaaacgtcaagtgggagcacaggtaagccaactccctcgttgcctggacaaggcaactagccagatgtcagcataagaggcgtagactcttgtccggaccaccatattttctcatccttactttgggtgagcttttgccgcccatgttcaagtccacctgaggtcaattaactggactctagggagaaggcagtcttggcatatttttaaaacgctacgggtgattccttatgcaatagaggttgggaactgtagttaagagtgttccagacgggaatcgtggtgcggatagtagctgctgagcatgtgtgagaggccctgggttagatcccagcaccaccattaaaagaagggggaggggttgtgttgggttgtgtgggttttgtttgtttgttttgaactttaagcctggcatggtggtgcaagttggtgtcaaggaaaatctgaggctgcatctgatgcctctaagtacaagctgggttaggagccagccagccagagccttcggggatgcggtgactccaaagcgaggtttgtagttcccttgcaggagaggagcctagttgtccagatgagtacatttcttgcttttaaaaatgtccttctgtttattgatttattgagatatctgtcgtcctaatgccgaaattataggcctatacaatcacccagtttatgtggtgcagggggatcaatcccaaaactttgttcttggtctagccaggccctctgtcaactgcactgtactgactgcgctacactccccgtccttacacccatctttgcacagcatcttactgcatagcttttgtctggcctgtgacttgctaggtagccttgaacttgtacagatccccttgcttcatgagtgacaggtttaagtacaagccaccatgtctggcttttttttttttttttaattaagaaaaaaaaatggggctggtgagatggctcagtgggtaagagcacccaactgctcttctgaaggtctggagttcaaatcccagcaaccacatggtggctcacaaccatccgtaatgagatctgatgccctcttctggagcgtctgaagacagctacagtgtacttacatgtaataaataaataaataaataaataaatcttaaaaaaaaaaagaaaaaaatgtggtttgtgttatgagtgtgtcacagtgtggtgtgaaggtcagagcacaactctgtggagccagttctctccttctgcctttgtgggggctctggtgataaaactcaggccaccaggcttataagacaatgcacccttactgtctgagccttttttttttttttttatggtttttcgagacaggctttctctgtgtagccctggctgtcctggaactcactttgtagaccaggctggcctcgaactcagaaatccgcccgcctctgcctcctgaatgctgggattaaaggcgtgcaccaccatgcctggcacttttttaataattaaaaacctgagaatttgggctggagagagaatgactcagtggttaagagcacggactgctcttccagaggtcctgagttcaattcccagcaatcacatgatggctcacaaccatctgtaatgggatccaatgccctcttctggtatgtctgaagacagcgacattgtactcatatacataaaataaatacatttcttttattatatctaagtacactgtagctgtcttcagatgcaccggaagagagcgtcagagctcattaaggatggttgtgagccaccatgtggttgctgggatttgaactcaggaccttcagaagagcagtcagtgctcttaaccactgagccatctctccagtcctaaatgtgtctttaaaaaaaaaaaagttaaaatttcaagacttttgagctggtgagatggctcagtgggtaagagcacccgactgctcttccgaaggtccgaagttcaaatcccagcaaccacatggtggctcacaaccacccgtaatgagatctgatgccctcttctggtgtgtctgaagacagctacagtgtacttacatataataaataaatcttaaaaaaattttttcaagacttttaaattgctgggcagtggtggcgcatgcctttaatcccagcactcggaggaagaggcaggcggatttctgagttcgaggccagcctggtctacaaagtgagttccaggacagccagggctatacagagaaaaaacaaaaaacaaaacaaaaacaaaaaaaatcaagacttttatgtgtttctgtggacatgagtgcaggtgcccaagcaagccagatgcactgctttacctgggtgctgggaattgaactctggtcctctgaaaatggcactcagttgcgggacctttttttatttatttttttattttattttattttaaatcatgtgtgtgtttgtgtgtgagcttgcctcaggagttgtcagatctctcgagtgggagttacaggctttgtaggccaaaagaggtgatgctgggaacggaactcaggctctgtgtaatgaatgctctttcttaacgctgagccacctttccagcctctcatccatcttcttccatttcctcctacatgctacttttagaaccgcccccgaacccccaccaactctaccgccctctccttctcctcctagttagatgttctgtgacgtttgccttacgtcacagccccgccctgcgaacttcgggcatgctgccagtgttcttagctctgaatgcagccggccaccccccctaatgcaagcacaagttgctgggactgactgagcggagatgagtgactggaagggctacatcagtgcagtgctgcgggatcagcggatcgatgacgtggctatcgtgggccactcggacaatcgctgtgtgtgggcatcacggcctgggggtctgctggctgccatctccccgcaggaggtgggtgtgctcaccgggccagacc

SEQ ID NO: 55 - RNA, 23 nt, synthetic construct
  source 1..23: mol_type = other RNA
taaagcactttattgagtttctg

SEQ ID NO: 56 - RNA, 23 nt, synthetic construct
  source 1..23: mol_type = other RNA
  modified_base 1: mod_base = OTHER; note = thymine
taaagcactttattgagtttctg

SEQ ID NO: 57 - RNA, 22 nt, synthetic construct
  source 1..22: mol_type = other RNA
aaagcactttattgagtttctg

SEQ ID NO: 58 - RNA, 21 nt, synthetic construct
  source 1..21: mol_type = other RNA
gaaactcaataaagtgcttta

SEQ ID NO: 59 - RNA, 21 nt, synthetic construct
  source 1..21: mol_type = other RNA
aactcaataaagtgctttgaa

SEQ ID NO: 60 - RNA, 23 nt, synthetic construct
  source 1..23: mol_type = other RNA
  modified_base 1: mod_base = OTHER; note = thymine
  modified_base 22..23: mod_base = OTHER; note = thymine
ttaaaatctacagtcataggatt

SEQ ID NO: 61 - RNA, 23 nt, synthetic construct
  source 1..23: mol_type = other RNA
  modified_base 22..23: mod_base = OTHER; note = thymine
ttaaaatctacagtcataggatt

SEQ ID NO: 62 - RNA, 20 nt, synthetic construct
  source 1..20: mol_type = other RNA
taaaatctacagtcatagga

SEQ ID NO: 63 - DNA, 22 bp, synthetic construct
  source 1..22: mol_type = other DNA
ttgttgtaggatatgcccttga

SEQ ID NO: 64 - DNA, 21 bp, synthetic construct
  source 1..21: mol_type = other DNA
gcgatgtcaataggactccag

SEQ ID NO: 65 - DNA, 31 bp, synthetic construct
  source 1..31: mol_type = other DNA
agcctaagatgagagttcaagttgagtttgg

SEQ ID NO: 66 - DNA, 20 bp, synthetic construct
  source 1..20: mol_type = other DNA
gctgattagagagaggtccc

SEQ ID NO: 67 - DNA, 20 bp, synthetic construct
  source 1..20: mol_type = other DNA
tcccatttcaggagacctgg

SEQ ID NO: 68 - DNA, 16 bp, synthetic construct
  source 1..16: mol_type = other DNA
ttcaaggtgaactgtt

SEQ ID NO: 69 - DNA, 16 bp, synthetic construct
  source 1..16: mol_type = other DNA
tatttctgctccaggt

SEQ ID NO: 70 - skipped

SEQ ID NO: 71 - DNA, 12 bp, synthetic construct
  source 1..12: mol_type = other DNA
tttttttttttt

SEQ ID NO: 72 - DNA, 20 bp, synthetic construct
  source 1..20: mol_type = other DNA
gccaggctggttatgactca

SEQ ID NO: 73 - DNA, 13000 bp, Mus musculus
  source 1..13000: mol_type = genomic DNA
actgcacagtgagacccttctatacaaagtaaaagacagttaggcatgcagctcagaggtaggttgtctacctagcaagcttgaggtcttgagttcaatcttttttttttttttttttttttttggttttctgagacagggtttctctgtgtagccctggctgtcctggaactcactctgtagaccaggctggcctcgaactcagaaatccgcctgcctctgcctcccgagtgctgggattaaaggcgtgcgccaccacgcctggccgagttcaatcttttaactttggaaaagacccacgaaacaaaagaggatgagaatcagagtggtttggaaattaccaaagcctgtttgtgaagtaaaataaaagttaagaagggggaagacagtgggcatttgggccctagcaataagaaaaggtaatccagaatgttctctagaaatattcccgtggcgttcactgggaactcccttgctgccttcataaaccatgtcttttggtagtttctggagtgaccctgctgcctgtttctctctccaaaccctgggagttgcccagtgccgcacagccattccaaaagcaaggaaaagtttgtatgatgtgtgaaggaatcaagaagatggacttacagacaagcactttagggggcgagggaaggaagggggaaaggcttgcctagtttcccttatctgcagggtttctaaaacaacaacaacaacaacaacaaaacaaatctccatagtttatgccagaagtgagccccgcctttaatcccagcactcagaaggtagatcctgacagatctgagtttccaggccagccagggatacagttttaggacaatatggcagtgagactctgtctcaaaacatgtcaccctatacactttatcattttttttaattccagacagggtttctctgtgtatctctggctgtcctggaactcactctgtagaccaggctggccttgaactcagaaatctgcctgcccctgcctcccaagtgtgccaccactgcctggtgactttatttttattttattttttaaagaaacaacaacaaagcgtggggtctcctatctgtcttggctcacctggaaccctctatgtagaacagccggccccgaacttgcagagatccagccctaccattttaaagattggtttcatttccctggcctccagaacggtcttagaccctccccccagcagaagcaaagcagaaacagaaggtcgggggaggagagggaggtgggtaggttaagaggaacgatgaagaacaccttctgctagttaactttgcctaattatagatgaatcacagatgcccgacactcagatcaccgcccataatttaggtgactcaagtttttgtcctttcagaactaaaattctaaaaaatttccaggcccaaagcatccatcaagtgtgcctctagagaggaaccgtttgcactcagtaacattccattctttagggcttcttacagacagaaaaagagaaaagtagggcagttttgcaagggtcacctgaaagctgcttaaacaagccccaggcttggtacacacagctttaatccagcacttgtgtaaggacctgagtttgaggccagcctggtccagaaaccctgtctcaggaaaaataaataaggaagaaagaaaaagcttaaactggccattgggatgggaagggaggcaatggtctaacactggacctccagctttgctagagccccacctcagtctaagggtgcctctctcctttagttttataatagaatattcatgtaattttatgtccttagtgactatcagcctgtatggtcattaagttctgtctaccctgggaaagcctctgcccaaacctccctattcacaggtcctagaacgtagaggtggggagcaggacggtgccgccaggccgtgtgcgatcgcgagctctggatctcaatgcgccccggggcgctgtttcccacgactccagcagcttttctaaaaatccaggcagcctccagtttacgggatcaacccgagactcgcttccctttgaaaattctagagtataaagtaaacgtacgagcaaagtatgtgtcttaacacttaatggatgacatagagaccaaaaaagccatgtccgtgggcccagtaggccgcaataaggggcgaccaggaaactgcagcacagccccccgcagccgccctgctcccacaccagtcattccagcaccgtggtgaaggcgcttgggggcggggcggggcgcgcctgcgcagcgaggctctgcagcagaaactttgcctagaccggctggaaccggttagaaccggtcgaacccggccggctgccagccctcgattcagcagctcacaaagggaggcggcgactcacgacccgcgtatccttgcgcctctccccaccccctttgtcctcgcgacgggttccgcggtcctccccgccctccctggcgcggcccccgctttctgcgcccagtgacgctttctccatggtcctgggagaaagagaaaaacatcctttcccctccgtcgtagttttaggaagcgatgagatagacctggggaccttgccgccacgggccgggctctgacggttattagcgcagtgcgggtggtgctcggcatggccgccaaggtcgccgtgccctcacctgcagcgaccatggccttgctgggctgagaccgcagcctaacatggcggacgtaggcaagcaccaaagcgctcgtgtacccgggctcggaaaagtggccccgagagcagccggaggctgcaggtcgtccctacaggagcattcccagtataaaccagtacaaagtgtcaccacctcagaagccactcgcagggccggtcactttccgagagacctccatcttgtttcgcatgaaatggcagccgctcggggagttacaaaatgggaagtggaagctgaagctgtgggaaagcctgttttaacacttgcaacatacgctataccctctgtcctcccaggaaaacgcaaaaggtgttgaaacatctgaaaaacttggggctcccatttttaatagctattagttcatgttttttctccttgtgaccagaaattttaaacctatttgtacctatttagctggtacaagctaaacatttctctgtattagcaaggtccaagaggcccacacgacgtcaagaaaaatctagaaacttggaagtcaggatctatttttaactctctgaggaactatttttcttccttcaccaaggtggtggagggttactaggttccggtggagtgacgtgtccctttgcaataaataccggcgctccgggctctgcgtcaggcattcaggcagcgagagcagagcagcgtagagcagcacagctgagctcgtgaggcaggagactcagcccgaggaaatcgcagataagtttttaattaaaaagattgagcagtaaaaagaattagaactctaaacttaagctaatagagtagcttatcgaaatattacttagtcttaataatctaagaagatcttaagagataacatgaaggcttatttaaacagtttgaaaaaggaaatgaggagaaaagtatttgtactgtataatggaggctgaccagagcagtttaggagattgtaaagggaggttttgtgaagttctaaaaggttctagtttgaaggtcggccttgtagattaaaacgaaggttacctaaatagaatctaagtggcatttaaaacagtaaagttgtagagaatagtttgaaaatgaggtgtagttttaaaagattgagaaaagtaggttaagttgacggccgttataaaaatccttcgactggcgcatgtacgtttgaaggcatgagttggaaacagggaagatggaagtgttaggctagccgggcgatggtggcgcacgcctttaatcctagcacttgggaggcagaggcaggcggatttctgagttcgaggccagcctggtctacagagtgagttccaggacagccagggctacacagagaaaccctgtcttgaaaaaacaaaaaggttaggctagtatttggagaaagaagattagaaaatggaagtgaaagacgaagaagacatacaggaaggtgaagaaaaagctgttagagaagataggaaaatagaagacaaagcatctttagaagacagaaaaggtacttaaaggcacaggtagtaggaagccgaagaatagaagatagaaagaagcaagatagaaaaacaaaatggaagttaagacaactttggatgccagcattcaagataggcaaagaagataagattgaggccaaaaggttggataagatataaagtcagaaggaaattatctttaaagccataagttcaaatttctgatggagcgagcagtttagaagagtctttagacagccacatacaagattgaagctagcaatcaaagctactaggactgaagtaaaaagttaaggcagaatgcctttgaagagttagaagaatattaaaagccttaacttgtagcttaattttgcttgatgacaaaaggacttttgataacagtttcaagattgtcagcattttgcattggacttgagctgaggtgcttttaaaatcctaacgactagcattggcagctgacccaggtctacacagaagtgcattcagtgaactaggaagacaggagcggcagacaggagtcccgaagccagtttggtgaagctaggaaggactgaggagccagcagcagcagtgcatggtgaagatagcccaggaaagagtgcggttcggtggaggaagctaggaagaaggagccatacggatgtggtggtgaagctgggaaagggttccaggatggtggagcgagagcgagttggtgatgaagctagctggcggcttggcttgtcaactgcgcggaggaggcgagcaggcattgtggagaggatagatagcggctcctagaccagcatgccagtgtgcaagaaaggctgcagggagagcatgcggtgcggtaacattccttgaggtcggcaacatggtggtggttttctgtaacttggatggtaacttgtttactttgtcttaatagttatgggggagttgtaggcttctgtgtaaagagatatatctggggctgtatgtaggcctttgcgggtgttgtaggtttttctttttcagggttatgtcctcttgcatcttgtcagaagcttttgagggctgactgccaaggcccagaaagaagaatggtagatggcaagttgtctttaaccgctcagaggggaatgaatggtagagccagcacaacctcccagttttgtaagacgttgtagtttgaacagatgacctaccacaagcctcactcctgtgtaggggaggtaattgggcaaagtgcttttgggggaatgggggcaaaatatattttgagttcttttccccttaggtctgtctagaatcctaaaggcagatgactcaagggaaccagaaaaaaggaaatccactctcaggataagcagagctcgccaggtttacagtttgtaggaagtagaggatggatgctagctttcacactgagtgtggaggagctggccatggcggaattgctggtagtttactctttccccctcccttaatgagatttgtaaaatcctaaacacttttacttgaaatatttgggagtggtcttaacagggaggagtgggtgggggaaacgttttttttctaagattttccacagatgctatagttgtgttgacacactgggttagagaaggcgtgtactgctatgctgttggcacgacaccttcagggactggagctgccttttgtccttggaagagttttcccagttgccgctgaagtcagcacagtgcggctttggttcacagtcacctcaggagaacctcaggagcttggctaggccagaggttgaagttaagttttacagcaccgtgatttaaaatatttcattaaaggggaggggtaaaacttagttggctgtggccttgtgtttgggtgggtgggggtgttaggtaattgtttagtttatgatttcagataatcataccagagaacttaaatatttggaaaaacaggaaatctcagctttcaagttggcaagtaactcccaatccagtttttgcttcttttttcctttttctttttttgaggcgggcagctaaggaaggttggttcctctgccggtccctcgaaagcgtagggcttgggggttggtctggtccactgggatgatgtgatgctacagtggggactcttctgaagctgttggatgaatatagattgtagtgtgtggttctcttttgaaatttttttcaggtgacttaatgtatcttaataactactataggaacaaaggaagtggctttaatgaccctgaaggaatttcttctggtgatagcttttatattatcaagtaagagatactatctcagttttgtataagcaagtctttttcctagtgtaggagaaatgattttccttgtgactaaacaagatgtaaaggtatgctttttttcttcttgtgcattgtatacttgtgtttatttgtaacttataatttaagaattatgataattcagcctgaatgtcttttagagggtgggcttttgttgatgagggaggggaaacctttttttttctgtagacctttttcagataacaccatctgagtcataaccagcctggcagtgtgatgacgtagatgcagagggagcagctccttggtgaatgagtgataagtaaaggcagaaaaaataatgtcatgtctccatggggaatgagcatgagccagagattgttcctactgatgaaaagctgcatatgcaaaaatttaagcaaatgaaagcaaccagtataaagttatggcaatacctttaaaagttatggcttatctaccaagctttatccacaaaagtaaagaattgatgaaaaacagtgaagatcaaatgttcatctcaaaactgcttttacaaaagcagaatagaaatgaagtgaaaatgctgcattaagcctggagtaaaaagaagctgagcttgttgagatgagtgggatcgagcggctgcgaggcggtgcagtgtgccaatgtttcgtttgcctcagacaggtttctcttcataagcagaagagttgcttcattccatctcggagcaggaaacagcagactgctgttgacagataagtgtaacttggatctgcagtattgcatgttagggatagataagtgccttttttctctttttccaaaaagacctgtagagctgttgaatgtttgcagctggcccctcttaggcagttcagaattttgagtagttttcccatccagcctcttaaaaattcctaagccttgcaccgatgggctttcatgatgggatagctaataggcttttgcatcgtaaacttcaacacaaaagcctacatgattaatgcctactttaattacattgcttacaagattaaggaatctttatcttgaagaccccatgaaagggatcattatgtgctgaaaattagatgttcatattgctaaaatttaaatgtgctccaatgtacttgtgcttaaaatcattaaattatacaaattaataaaatacttcactagagaatgtatgtatttagaaggctgtctccttatttaaataaagtcttgtttgttgtctgtagttagtgtgggcaattttggggggatgttcttctctaatcttttcagaaacttgacttcgaacacttaagtggaccagatcaggatttgagccagaagaccgaaattaactttaaggcaggaaagacaaattttattctccatgcagtgatgagcatttaataattgcaggcctggcatagaggccgtctaactaaggactaagtaccttaggcaggtgggagatgatggtcagagtaaaaggtaactacatattttgtttccagaaagtcaggggtctaatttgaccatggctaaacatctagggtaagacacttttcccccacatttccaaatatgcatgttgagtttaaatgcttacgatcatctcatccactttagccttttgtcacctcacttgagccacgagtggggtcaggcatgtgggtttaaagagttttcctttgcagagcctcatttcatccttcatggagctgctcaggactttgcatataagcgcttgcctctgtcttctgttctgctagtgagtgtgtgatgtgagaccttgcagtgagtttgtttttcctggaatgtggagggagggggggatggggcttacttgttctagctttttttttacagaccacacagaatgcaggtgtcttgacttcaggtcatgtctgttctttggcaagtaatatgtgcagtactgttccaatctgctgctattagaatgcattgtgacgcgactggagtatgattaaagaaagttgtgtttccccaagtgtttggagtagtggttgttggaggaaaagccatgagtaacaggctgagtgttgaggaaatggctctctgcagctttaagtaacccgtgtttgtgattggagccgagtccctttgctgtgctgccttaggtaaatgtttttgttcatttctggtgaggggggttgggagcactgaagcctttagtctcttccagattcaacttaaaatctgacaagaaataaatcagacaagcaacattcttgaagaaattttaactggcaagtggaaatgttttgaacagttccgtggtctttagtgcattatctttgtgtaggtgttctctctcccctcccttggtcttaattcttacatgcaggaacattgacaacagcagacatctatctattcaaggggccagagaatccagacccagtaaggaaaaatagcccatttactttaaatcgataagtgaagcagacatgccattttcagtgtggggattgggaagccctagttctttcagatgtacttcagactgtagaaggagcttccagttgaattgaaattcaccagtggacaaaatgaggacaacaggtgaacgagccttttcttgtttaagattagctactggtaatctagtgttgaatcctctccagcttcatgctggagcagctagcatgtgatgtaatgttggccttggggtggaggggtgaggtgggcgctaagcctttttttaagatttttcaggtacccctcactaaaggcactgaaggcttaatgtaggacagcggagccttcctgtgtggcaagaatcaagcaagcagtattgtatcgagaccaaagtggtatcatggtcggttttgattagcagtggggactaccctaccgtaacaccttgttggaattgaagcatccaaagaaaatacttgagaggccctgggcttgttttaacatctggaaaaaaggctgtttttatagcagcggttaccagcccaaacctcaagttgtgcttgcaggggagggaaaagggggaaagcgggcaaccagtttccccagcttttccagaatcctgttacaaggtctccccacaagtgatttctctgccacatcgccaccatgggcctttggcctaatcacagacccttcacccctcaccttgatgcagccagtagctggatccttgaggtcacgttgcatatcggtttcaaggtaaccatggtgccaaggtcctgtgggttgcaccagaaaaggccatcaattttccccttgcctgtaatttaacattaaaaccatagctaagatgttttatacatagcacctatgcagagtaaacaaaccagtatgggtatagtatgtttgataccagtgctgggtgggaatgtaggaagtcggatgaaaagcaagcctttgtaggaagttgttggggtgggattgcaaaaattctctgctaagactttttcaggtggacataacagacttggccaagctagcatcttagtggaagcagattcgtcagtagggttgtaaaggtttttcttttcctgagaaaacaaccttttgttttctcaggttttgctttttggcctttccctagctttaaaaaaaaaaaagcaaaagacgctggtggctggcactcctggtttccaggacggggttcaagtccctgcggtgtctttgcttgactcttatatcatgaggccattacatttttcttggagggttctaaaggctctgggtatggtagctgatatcactggaacactccccagcctcagtgttgaactcttgataattaactgcattgtctttcaggttatgcccaattcgtcttattacctctgagtcgacacacctcctactatttattgaatactttgattttatgaaataaaaactaaatatctctcattgtgtgcttctttgtgcataaaacacaggcttattttaagcctaaagagaccaaatgtctgatctacctcagcttctccgattagtgaggccttccctgtttccttgggctgcatggctctttcatgcagatggctctaaagttgggcttgggtcctaggtggccactcttgcacctcaggaacacaaggcctttccctgctgttcaggctctcctccctgagaaaacattctggattgtctatgaggaagttgggaaaagatggtgtcgaaaagaggtggtgtgcattgctcctctgttcctaacactggatggaagactagttttcatgtagtttagggaaatagttatacatggtctaaaggcccaaaaacattcccagagtgtatgcaatactgtgtgtaagtgtgcactgcgtgtgtttggaggtcagaacttctctgaggttctagagatgaagcaagtcctcagccatggcccaagaatgggaaggaactgggtcctgctgtaccacttcccattccttaaggaacagtttggcccggtgtggtgcaagcatggtcggtcactgaaaaaagaaaacccacttaggtttcacaggcttgaagagctgcatgtcatccagcaaattactggctgctgtaaggacaggcccctaggtcccagtcccaggtgcccttcctgccactcaatcaagccttacaccctgggcaaaaacatcctgcgttgaaggttcagctcccagggctggaaacttgtgctggcatctaccccagttcaaaggggctcagcacattgacaactaaaactaagccctcaggtgagcaaaatggtctccttaaggcaatcatggtcattggtgttcctgcagtaaaggacagcatcacagctgatgtctgtgtactggctagttttgtatcaacttgacacagctggaattatcacagagaaagcttcagttggggaagtgcctccaagagatcctccacgagatcctgctctaaggcattttctcaattagtgatcaagggggaaagaccccttgtgtgtgggaccatctctgggctggtagtcttggttcagttctataagagagcaggctgagcaagccaggggaagcaagccagtaaagaacatccctccatggcctctgcatcagctcctgcttcctgacctgcttgagttccagtcctgacttccttggtgatgaacagcagtatggaagtgtaagccgaataaaccctgtcctccccaacttgcttcttggtcatgtttgtgcaggaatagaaaccctgactaagacagtctgagacctgacagatctgtgctaaagtctggtaccaactgagctagaccctgccacacacctcagtaatggcccattctgaattcacccagagctgaggctttgccgaggtgaggcacaaagacttcactggagagcaggagatatgaacagaggttggggctcacacttcctgattgggggccaggactgggggcaagatgaaggaacggtaggcatgcttgtaaatttcccaaagggttagatccagatcttagctttcagtgtgtaggccagggtgactctgaatttgggtccttggacctcaactttggaagttgcagggatgagccactgggctctgtacagtcttgtgctgcccacatgctctagttgtacaaaggatactccctcaaacaagctctccccaaataggcctttgacgtctggacccagccaccggcccaaaagccgattagtacagggccagcagcatagcatccttctactacggcatgaaattaggagaggaagggtttgaagagagagagtggaagatgtggtttttattttttatttattttatgtatgtgagtacactgtagctgtacagatggttgtgaaccttcatgtggttgttgggagttcacttttaggacctctgctcactccagtcatctcccctcgctcagtccctgctcactccagcccaaagatttatttattattatacataagtacactgtagctgtcttcagacacaccagaagagggcgtcagatctcattacagatggttgtgagccaccatgtgggtgctgagatttgaactccggaccttcagaaaagcagtcagtgctcttaactgctgagccatctctccagcctgtgatttttatattttaggtagggccatagtatcaccagacaggtattccttgtatggctcaggatggtaaaattaaagttgtgcctcaggctcctgagtgggactgcatggctgctccagtatacctggccacctggattgttgactctgaagctcaggtcttagtttctgcctctgatcagttgacaaacttgatacctgcccagctcacaatgcaacctttgcatgcatcctggacttagccttgacaagggtagataggcaactgagtaggacgtgactggacttcgctgccatctagtgtgcaaatgtagtgcatgcaatgtccttgaactttttcttgccatgcctattttgttgttttatttgtttgtttttggtttattaattttttgtaggatctctctaatctgacttcttggaatttgctatataaactgggctggccttgaactcacagcagtcctcctgcctttgcctcctcagcaaatcctgccagcctatatacctccatg

SEQ ID NO: 74 - DNA, 21 bp, synthetic construct
  source 1..21: mol_type = other DNA
cggatgaagagaggcatgttg

SEQ ID NO: 75 - DNA, 18 bp, synthetic construct
  source 1..18: mol_type = other DNA
ttggccacaccgtccttt

SEQ ID NO: 76 - DNA, 23 bp, synthetic construct
  source 1..23: mol_type = other DNA
agacctgggcaatgtggctgctg

SEQ ID NO: 77 - DNA, 20 bp, synthetic construct
  source 1..20: mol_type = other DNA
ctcctcagaccgctttttgc

SEQ ID NO: 78 - DNA, 24 bp, synthetic construct
  source 1..24: mol_type = other DNA
taacctggttcatcatcgctaatc

SEQ ID NO: 79 - DNA, 20 bp, synthetic construct
  source 1..20: mol_type = other DNA
ccgtcatgccgacccgcagt

SEQ ID NO: 80 - DNA, 52000 bp, Mus musculus
  source 1..52000: mol_type = genomic DNA
ttgcagtgactcagcctacctccttcttacgcaacatttttttttctttgtccaacctttactccttccttatttctcccctgcaatacacagggccaggagtctcctagaccccagttcaaatctgagctctaaatgcctacacagtgagttccaggccactcaagggtacatactgagaccttgtctcaacatacaccctcataaaaagagaagacagccacaggattctatttatatggaatactatactgagggctgagagagatggcacacagccaagcctgatgacttgagtttgattccaaacccacgtggtagaggggaagaactgactcctacaagtggtcctctgacccccgcacagatgctgtgcatgccccacaaccctaggaacaaatacaatcatagaaaataaatgtccagagcaagcaaagccacaaatgcagggagctggtggcagggatattaacaagatcccttttccaggataatggaatgtcttggagctagagaggtcataactgagaatatattaagtggcactgaattgcaaacatcaaaatctcaaatatgtacatttatataatataaattatacctcaacttaaaaataatcaagaatcaggtgtggtggtgcacagtgcatgagcataggagacctcaaaaatggccccacagtgacacacttcctcaaaccaacaaggccacatctcctaatggtgccactccttatgaaccaaacactcaaatacatgaataggaagctattcctattcaagccaccacactgcgcttggcagcacatgcctacagtctcagtattagggagttggaggtatattcaagatctcaaggtcagtccggactacactgtgagttcaaggggagtccgagctgcattcaaagaccctgtgtcaaaacaaggggagtggctgatttggtagagtagttggtcaacatgctcaaggccctaggcatcatcacagcactgcaaaataaataaacagaatattgagataagaggaagaaaacgtcaccaactcagcctaagccgtgaaagtttaataccaccgatactaggaaaagatggcaggtaccctgataatatgccctgggaaggatacggggattctgccaaagacgcataaagttaatcttgtagaactcgcaggtcattgcaggtccaagaggcactctttcaaacaactgacctcaaactaaggctgaggtgaagatctgggatccagtcttcggtccacaaagaagctataagggatgttattcggaataaccataaaactttgagtagaaacttgggtgaggactggagatgtggttacatggtagagtgtctcccttacacactggaaaccccaggttctggccccagcatcacaaaaatcaggcatggaagctcatacccttaatcctagcactgggaggcaaagccaggaagactgggacttctaggtcattctggtttcagtaagttcaaggccagtctgggttgcacgagactctgcctgaaaaggagaagcccccaaagtaggacaacaacgaagtgggcctgaaagactgttcagttgttaagagagcagcagcagcagcaggaggatctgatttcagattccagttgtctcatgtaacaagctggctattccacaaatgaccataactccagctttgaggggtgcagtgccttccctggtctctgcatatgcaggtactcacactagtgcacacacacacacacacacacacacacacacacacacacacacacacagagaaacacacacacacaaataaatctttaaaaagtgtatccgtgactgtcctggaatttgctaggtagaccaggctggctttgaattcacagatctatctgcctttgcctctggagaccgtgtgaaccatgacatcttccccaattaacaaaaatacacttatttattatgtagccagttgggggggggggcgcggcagggaggggagatgggccctctacaatgtagagatcagaggacaacttgtggaagttgttctctcctcccactacaggagtgtctgtgtatgcccttccaggattatatgggtcctctgtttcagtcctttggtctcctagtgatggagatgttcaagaacggcacccttcacccgactcctgagatttcccctgaatgcacctctagagcttccattctcacttcacacagagggagtgttcagagcctttgcaggtctcctggatgtaaaactcagtgaatggcaacgaaatgataggcctcatggtaaaaccatgtagagtgtgggatgtgctaggagctggtgtggattttttttacaaagtattttatcttatttcttgcgcacataagcgtgtgtgtgtgtgtgtgtgtgtgtgtgtgtgcgcgcgcgtgcgtgcaagcattcgagcacagtgtgtgtaggagtgcatgtgtatcacggggtacatgtggcagtcagaagacaatgtatgtgttggtttttttattttagcatgggaggcccaggaattgaactcgggtgtctgtgattggcagcaagcacctttccctcttcggtagacctttgtgatcttcacattgtaggagagctgcacacaggtacatgagactcatatccacgaaacccctggccagattgtaaatgctgcccgcccgggtttgtggcgcagttggagcctgagttttgaggtgattgatgtttagtgtccccatgaaacttctatatggtttgcatgggtgcggctacttttgttcattggcctctctacacccaggtttagagggtttggagtcaggggtgtttgcgtttgtgtcatgactgtgtgtgtgagccctggctttggttgtgtgctggaggataaggatggatgtgctcacgagctggatacctgtcacgtcacgcaggttaagcccgcatctcttgtggcagacctgagtgtaccgaggtctgcaagtcccaggcttggcgggtagaagggtcggaccccaggaggccgggcagggggcgggggcgctcagagccggccccggggcggggcgagccgcccaggcctgggggcggggcagcccgacagcctcacttcggcgaagttggcggcgcggaggctggcccgggacgtgcccggagccctgggaaagagggaggagggaggagggagggtcgtggccggccgccatggggctgggggcccggggccgccgccgccgtcgtcgcctgatggccttgccaccgccaccaccgcccatgcgggcgctgcccctgctgctgctgctagcggggctgggggctgcaggtgagggatcagggactcggagggtgggactggggttgcaggggcggggaagcaaaaaaaaaaaagtatatatatatatatatatattctcctaagtttggaatttagaggacctgcccccccaggattaaagccggggcagagatgcagcagctgggggtgtgtatcagtcatggggttcgcacagattcatgatgggaaggcaggatcctagaggcccaaggaatcgagacccttgaatgaatcaagagtccaagagtaggagctggagttgtgggaatctggggctgaaattcggaagcgaggtgtaccaaaagtttcaggctcaccccatcctacttctgcctggagtcctgggaaggttaggatgggggctcaaggaccccccaaggcctaggaccccttgcacaaaagcgcctcccctatcccgggcgggccgtgaaggggggggggggacaaggagggggcgcggagttctcggagctctgaactcggagaaaacgtctcatgttggagagctgcaagacccggcgctggagcgcgagaagcaggagctggagcccttgcgaagcccgcgccccctcccccgcgtgccgcccgatccctcttcaggccgccgggatcccgtagtctcgggaccccgtctctgtgccgggaggagaaggggcgaggtccacgtgctcatctccagcttctgggcccccatccctgtggccgcagcccctccccagcatgcctgggcccccctcccctcttcctccactctgagctcccctcccccgctcgggacaatggcttcgccgtctagacaccccctccccccggccggcctcacgctttctttgccagacaaagcgggacccacgaagggccagagcggggactgagggggcgcccccacccctgccccgggaggcctgatcagcgggggaggggcataggcagttggctgtggccaggggttctagggctcctatggtgggactcagtgctgggccgggtgagagttgcatagagagtggcttagcctctaggctctactaggcaggactgggcaggagccaccgcccccgcggggccagtgcagactcatggcctgtcacacatagaaagggaaaaaaatgcatgtgcacaggtttagtacagccatagatgcacacttggcacacacaagacacacctatgtgtttgctgctcacatgttgatgtgcattcaggcataggtcacccaagacctgctaggcattcatgttcaaggtcacatttatagctgccaatccatgacccagacaagcacaccagtggccacacagctgtatgcttgaatactgctgttcatatagatggatgcacagtgcatgcagctcgtgtgcacatggtgtacaaggctatgctgtttgaagataccagcatggtaccaagcacatgcactattagtcactattagtaatatagtggcctaatacacagactgtcacacaaactcacaggcatatgtacacacacacacacacacacacagagctcacatggttgtgttctcccccccccccatacagcttctagtgtgatgtcacaaaattgaataggtccttcattcttaactcatccttttactgtctctgttttttttttttttttttttttttttttttttttggttggtttctcctttcagtttctgtctgtccctatttctcaattctcccccaatctcctttccttctctctctgaggaacaaaacttctacaggggcccacacctgtctgagcacataattctccaccagctcagacctggccacacctctcaccatctggcaagactgccacacccaaagtgggatacaaccctgagggatggggacactggacagggcagcacatatcttgcaccagttctctcccttacactccatagcctatccatctagtacccaaaaccacctctctatggctgctgttggttcctcctaggtgtacgctttgcctactctttcaagagctagcagggactacagtagacacaggctcaacactgcctgccttctccatctacccaagacatgggcttgaagctgcagatcccctttaacactcagcatctttacttcctactctgtgccttttctctgttgtgcacatcctagtcaaggcaacttcctgagcccacacatctggcacaagtcaactcctccttctcacccctgccttgtggttcccagggcttgagctggcagggacagctgcagccccaacagctggggctggggtggggggccgtgggaactgtagatagggggcctcttagtacccacacagataccctcctagcccagtgcagctgttgcctggcagacaggaggggagggtctgatttggggttctgtgtgcctgtctgacctccccaccttcttttgcccccacacagcacccccttgtctggatggaagcccatgtgcaaatggaggtcggtgcacccaccagcagccctccctggaggctgcttgcctgtgagtgtctggcccagcgccatcagtgggccctgtgtggggagggaataggtcctctgcctctgagtctcctgggggatgctggtatcacttcctccatgtgtgtggcttcggtaacctcttgtgtttccccatagggaatcagacagctttaagagcagaagctctggtgtctctctggcttgggttcaaatcccgcatcatcttgctaagtgtctgagacttaaccagttcgttaagtcacagtgctctggaataaaagattgatttccaaggccaatgacagtggcttgacagtgacaaggacgtccatgccctcccatatgtcagaatacaggaggcacccaggagacctattttagagccctgttttattttgttctagtttgtatgacctttggttagtcatttctcttttctgaacctcacggcatctgtgaagcaggagtgctttccctggcctgtgactgtgcaggaagggacccaagaattatttgggaaatgcagaaagttagagaagaggaggagggtttgggagctaccagaggcaggcagaactctagattctgggacatctcgagcaagtgctggggaccttcccagccacttttgcatctacaatggcttttctttcatgaggttgactgtaaagatttgaaaaggacctttaactgggcttacgcttgtatttcccagcagaagcaggaggtcattccgtacagttccagggcagtctgggctctcacagagtgagcttgtctcaatgttttgttttctaaagggggctggggcagctgagagcttagtaggtaaaggcacttgctgccaaactggacaacctgagttaagtcctggggacccaaaagatggaaagagagaactaatctcggcaagttgttctctggcctctgcatgaatacatacacacacagataatgtaataaaaatcaaaactatattaaaaataagagaaacttttgaaaccttttgacgactggagatttcaatggtgtttgcccagtacatacaaagccctgggttggagggaaaggacctcaaggtctgtgggcttattggttttgttttctttttaatagtaatagcattccattcagcagctattaattagttcgttcatcagtgtgttcatttcctcaatactgtgtgggctagccagtggatcagcctcaagctaggtggggccaggatgacttccgatatagaaaaacaaaaacctttaaaattcaatggctggaatctcatttagctttattgggggtcactggccgaggggcatcctgaggctgagggttgggagggtccttacttggaagcagtcgcttggaagggaggctgaggcgtggggcggggcgcctgcgcagtagaactacacgcctggacagctgccgacaggtgaatacgcctgaggctgccccgcccctcgacaggtgaatcaccggctcacgcggcctccgggagcccggatcgcgcggagtggagctcgcttaaggctccagaatagagatttgggggggacctcagcctatgttcttctcatcccaacccactctttcaggcttctcaaatcctgctgtctccttaggagtgaccgccccctccccaatgacactttctcccccacttctccccagtcttccgaccgggagaaggcttcggagttctccgtcgcttggtccttgtctgtcttgctagtccgttccctccttggaagctgctcccccgcccccctcccgctccgctacgtctcccctaaagctaaggcggcgaggcgggtccggggctggaaccggccggaccggtcggcgggggcgcgaggtgcagagcgtgggaacccgcccgcggcgcagggagggtgccgcgcccagcttggcgatgatacccgtggtccccagtgctctgcacgcagcgccccctcctggcgtgtcatcgtcgccgccttgcaggactgggtagggggttgagagcatcacctccaaggttcgaatgtccaccacacccttgcagggttgagcttcgacagggttgagcttcgacacgtttatctaggaacggaattcggggtgggggtattgtagggtgacgctaagaaatgagtcacctgctttgagttctgcccttggtgtttgagacaagactgagctaggggatcctcgctgggagccggaagggtagggtagaggagaggagggttgaggtaggggtgatgtacgttgtgtcattgcatagttgcatagccaggattgtttgactactccagtccccttccttagccctcctcacctctgtccgtagaaaggttacaaacttaagtcgtcttttagtttttattttctagcactcaggacttagattttttcgagacagggtttctctgtgtagccctggctgttctggaactcactctatagaccaggctggcctcgaactcagaaacccgcctgcctctgcctcccaagtgctgggattaaaggcgtgtggcacccctgtcccactactcaggactatcttgatgggtttggggaaggggccgcaggccttggtccagattgagggctgaggtgcccatgccaagccccaccgatccatgagaggaggattgcatgctcttgggagaaaggagtttatgatttgtggctatcttcctgcctctccatatgagtgagagtttcccatattcgtgtgggaggctgtggcttcttgtctgtgtatgccaggccagtatgcccagttgtatgtgtctgtgcggtttctgggtctttctgggtgtgtctgcctcctggagtctgaggcttttcatttgttgttcccagtacctggaatgcccttccccacatcaccctttggctcacccatttttttgccgttttttgttgtttgaaaatataacacccactcattctatcacccttaggctgcttgtgagtttcctgtggttgctgtaccagattaccacaaacagggtggcttaaaacaacagaaatgtactctggaaaatcctggaggccggaagtctgaaattaaggcgttgccagaactggctttctttactgaccatccagggggagaagactttcttatgtcttgtccagtttctgattggctcaggcttatggctgccccaccctactctctgcctttgtctgcacatgacctttcttctctctttagtttctatctgtctctctcccgtgtcctgggaaaggaacctaggcccctgcacataccatacaagaactaccactgtgtcatgtccctggcttttctcttgtaaagatactgtgtgttggggctggtggcacgactcagtagtttagagcagagtttggtccccagctctcattggttactcacctgtaactccagctccaggggttttggcctctgaggaaggacactgtactcaggtgcacacatactcacacacaaccacacatgcatacaataaataggggctgaaaatggctccacagttaagagcgtgtattattttctcagaggacttggatttgatccctggaatccgcatgataactcataaccatccttaatgccaacgccctcttcagacctccatgggcatcaggcacgcacagcatgcacgtacatacatgcgtggaaaacacttatccacatcaaagtaaaataaatcttttaaaaattggcttgaaatacaaagtgaacttgtcttggtattcttcacaattacattggcaaaaattcttcttccctttctttttcttgtgctgagaattgaatctagggctttatgcatcctaggcaagtgcagtaccactgagctatattccaaatcctcttgttatttgtttaaaatatatgtattgatcttgagacagcgtctcatgcagtcaaggctagcctcaaacttgctatgtggccaaggctgaccttgaactcctcatcctcctgctttttcctcggaatgccaggtttgtgtgaaccaccttgcctggcacatctcctttaaaaagaaataaagtgatagtcacagcttctgggacatgggcgtgttcttcagtgtaccacgtcctttgtgagcttgtggcatgtacaagctgtttttggtatgttcgtattaatgtgttacctgtctatgttttctttgcctgacctggctatgaggaccaaaagggtaagagtcacttttattttgcttgctgctgtgttttagtatgtagtaggtgtcaataatatttgttgtcagaataaatgaggatggctgggaatggtggcacttgcctttaaacccagtgctagaggcagagaggcagaggggcagaggggcagaggcagaagagaggcagagaggcagagaggcagagaggcagagaggcagaggcagagaggcagagaggcagagaggcagagaggcagagaggcagaggcagaagagaggcagagaggcagagaggcagaggcagaagagaggcagaggcagaggcagaggggcagaggcagagaggcagaggggcagagggacagaggcagaggcagagaggcagagaggcagaggcagagaggcagaggcagagaggcagagaggcagagaggcagaggcagaggcagaggggcagaggcagagaggcagagaggcagaggcagaggcagaggggcagaggcagagaggcagaggggcagagaggcagaggcagaggcagaggcaaaagagaggcagaggcagagaggcagagggacagagaggcagaggcagagaggcagagaggcagaggcagaggcagaggggcagaggcagaagagaggcagagaggcagaggcagaggcagaggcagagaggcagagaggcagagaggcagagaggcagagaggcagagaggcagagaggcagaggggcagaggcagaggaagagaggcagagaggcagagaggcagagaagcagagaggcagaggcagagacagagagacagaggcaggtaaattactgtgaattccagtctagccagcgatatgtagtgagaccctgcctaaaatatattaaaaaaaaaaaaaaaaagggaaggaagagtaaatggatttgtctgatagtctgtctggcacgagtgttgtttgataaacgcatcttgtgttatctgtctggcattgccatgcttttataccgtcccgaccacacatcttcccacaggtgcctgccaggctgggtgggtgagcggtgccagctggaagacccttgccactcaggcccttgtgctggccgaggcgtttgccagagttcagtggtggcgggcaccgcccgattctcctgtcgttgtctccgtggcttccaaggtgaaggggtgtgtctggacgggaacccttggtaggcgagaatgtagtcagacccaagctcaccctctcctggttcttccaggcccagactgctcccagccagacccctgcgtcagcaggccctgtgttcatggtgccccctgctcagtggggccggatggccgatttgcctgtgcctgcccacctggctaccagggtcaaagctgccaaagtgacatagatgagtgccgatctggtacaacttgccgtcatggtggtacctgtctcaatacacctggatccttccgctgccagtgtcctcttggttatacagggctgctgtgtgagaaccccgtagtgccctgtgccccttccccgtgtcgtaatggtggcacctgtaggcagagcagtgatgtcacatatgactgtgcttgccttcctggtaagtaagttgtgcccagggaaggcagctggggacaataggctagcctcttagtgaccattgtcaccttgtcctcccctacgaggcttcgagggccagaactgtgaagtcaacgtggatgactgtcctggacatcggtgtctcaatgggggaacgtgtgtagacggtgtcaatacttacaactgccagtgccctccggagtggacaggtgggcatcagggctgcagagaaccagggtggctgacctcaggtgggcacacgggcaacttagactagcacatctttgtgccctaggccagttctgtacagaagatgtggatgagtgtcagctgcagcccaatgcctgccacaatgggggtacctgcttcaacctactgggtggccacagctgtgtatgtgtcaatggctggacgggtgagagctgcagtcagaatatcgatgactgtgctacagccgtgtgtttccatggggccacctgccatgaccgtgtggcctctttctactgtgcctgccctatggggaagacaggtgagtggcccttttctttgtaggcaacagaatggtttcagcatgaaaggtaaaaacagactctgagttgagcgttagaaagattgggggctggggatgtttcttcctggcagagtgtgtgcttagtgtgcacaggctctgagtttaatccttagcgtgaaggaagacaagaaggaggagggaaggtggaagaaagaaaggaaagagggaggaagggtttgctggaccctgggtttggaaagaagcctgagcctctgtcctatgaggtgcatagtccaaggcagagactgttggaattggggaactattgagaggtctaactgggaaaaaggcaggaactatggaagtcacaaaggtctgtttgtcccttacatcttattttggtggggggtggtcagactctggttgtcaggctaggtggcaagtaccttgagtttattgttgttgttgatgcgttgagacatggtgtcactatttgtacatcaggctagcctcaaacttgcaagaaaggatccttgggcttctgtgttctgagtggtgggattaaggaattttgttgctatgcctgaatagggtcttgatttatcatcttttaaaatattaaaaaaagtgtgtgtgtgtgtgttttggctgcatttatgtatgttatattatgtcatgctacacgcatgactggtacgctgagaggccagaaaaattcatcagctctcctgagattggagttactgatggtggtgagctgccgtgtaggtgtgttgggaatgaaacgtaggtcttctggaagagcagccaggattcttagccgctgagcacctctttgggccccggtgccttgtttgtaaaatgtttctaagttattttcaaatggtatcgaagaagcagattaacagataatactgaaccaatattccaatgtgaaacgtcctgaatgtttcactgtttcataataaatggcttttccaggacagccagggctatacagagaaaccctgtctcgaaaaaaaaccaataaataaataaataaataaataaataaataaaataatattaatagatggcttaaaaaaataagaacagatactaatacagtgctggttaatatacataagaaacaggaggcaagggccaccccaaagggtgctcagggcgtaaaggcactcgctgagttggcctggcaagcctacttctatcaagggaatccactggtagaaggagaaaaccaaccaagtttcctctggcttctacacatgcactatgacatgcacacactccccagataagtacattaggaatgatgatggtgatgatggtgatgataatgcaaggagctggaagcgtagctgagtgatggtattcattcgttgtatgctcatggtcctggggatccattaccagtaccagtcttcttcctcaaacgctaggtctgggggaactttgggccaccccgaggatcagcgcttcatttctgcttaccttttctcaggcctcttgtgtcatctggatgatgcatgtgtcagcaacccctgccatgaggatgctatctgtgacacaaaccctgtgagtggccgggccatctgcacctgcccacctggcttcactggaggggcatgtgaccaggatgtggatgagtgctcgattggtgagaagagtaccttctggaaaggagcctgaaaacggaggggtggggccatggctggccacgcccacactggctgtgtcttctcccccatattcccccttcttgcaggtgccaacccctgtgaacatttgggtcggtgtgtgaatacacagggctcattcttgtgccaatgtggccgtggctatactggacctcgctgtgagactgatgtcaatgagtgtctctccgggccctgccgcaaccaggccacgtgtcttgaccgaattggccagtttacttgcatctgcatggcaggtgggtggtgggtatggcttgggtgggtcatgaaggctggggcctggggtaaaacttggtttattgttatttacttgaagaaaaaatgctgggcatagagacacatgactgaaatcccagcacttgtgaggcagagataggctcatctctgtgagttcgaggccagtttggtctacagggtgagttctaggatggctaggattataaagtgagactctgtctcaaaataaataaaataaaataaaataaaataaaacaaaacaaaataaaaaggggatagagagatagcttagtggttaagagtcctccctgctttctagagggctgagtttggttctcagtacccatatggggcagtgcacaactatctagctccaggagatctacaatgctcttttggcctctgaagatacccatgtgtttgggacacacacacacacacacacacacacatgcatgcaaataaacaaaattaaacaaacaaataaacaaaagacatttcaaaagagctgaagtggcacagtaagacggtctggaactcactgtgtagaccaggctggccttgagctcacagagatccacctgcctctgcttcccaaatactggatcgaatggcatgtgcctctcggcctagtgcactttaaccatgagggttccaaattgtcaggcttggcagcaagcatccttatttgagccatcttgcttgcccatgactgagtttaaagtgaagcttcctggctggagaaagagagagagagttatgtgtgtttaggatgaaaccttgaaaactcgataaggtaggtgcagggtcagagcggattttaccagataacatcaaaggggagcttcataaaccttagtccatgaggggccacagctgggtgagggtccagcttgtttgaaactaaatctaagcctgaaagtgtcgtgcacgcctgttatcccagcacttgagcagctgaggcaggagcatcattagtttgaggccagcctcaaggccatagtaagaatttatctcaacaaacccacaaccaaatcaaagtccccaaaccaagtgaagaggccttctgagggaactttctgggccctcataccattcccttcaggcttcacagggacctactgtgaggtggacatcgacgaatgtcagagcagcccatgtgtcaatggtggtgtctgcaaggacagagtcaatggcttcagctgcacctgcccatcaggtgaggaccctgggacaaggagcctggtgtgtcaggttatgacaatgtggaacttaaaaaaaaaagtaattagttacttaactcttttgtgtgtgggggttctctccttccactatgttatgtacattttgggaattgaactcaggtggtcaagcttggctggcaagcatgtttatcttctgagccatctctctgggctagtctgtattgaaattaatttaaaacaaagccaagggggtttcccactcaaataaggcaggctgcctgctttaactgtttgtgtcacctttcatccactactcacttccaggattcagtgggtccatgtgtcagctggatgtggatgagtgtgcaagcactccctgccggaatggtgccaagtgtgtggaccagcctgacggctatgagtgtcgctgtgcagagggtgagggcggaccgtgagactgtggcaagagccagaaggtgggctggtgggccaatgggtgtcaaggaccaataacagacttggggatggcctcagctaggccaggtcagggccagtgacgctgatgatggaggtaggcagaggtcttggcaagattcagggtgcagctagcagtgagatttaaagtgggcgtttctgggtcaggaacagagcttggagctgggcagaatggaagggagaaggggtgaggtctgagagctgagctggaattgggctgagattacagccatggagaagtgggcagaccctcacctcccgttcttgcaggctttgagggcactttgtgtgagcgaaacgtggatgactgctctccggatccctgccaccacgggcgctgtgtcgatggcattgctagcttctcgtgtgcttgtgccccaggctatacgggcatacgctgtgagagccaggtggatgagtgccgcagccagccctgtcgatatgggggcaaatgtctagacttggtggacaagtacctctgccgttgtcctcccggaaccacaggtggggcctggggctgggctataacagtacgtgggggtgtgtgggggtctgtgatgaatttgtaactggtgcttgacaatagtaggtactcttgccatacttcttccctccctgtaggtgtgaactgtgaagtcaacattgatgactgtgccagtaacccctgtacctttggagtttgccgtgatggcatcaaccgttatgactgtgtctgtcagcctggattcacaggtgggtaggtggctgccatgtagtgggggggggggggcttgtaagatagggattaagacacaagtctcttgggtgtccccactttatttttttaaaaaaggaaatattacatttcatttattttgtgtatgtctggaggtcagagggcatctggggggagtcagttctctccgtaaaggtctcagggaccccactcatgtcatcaggcttggggacacgtgtgctttccccccagttaagtccctttcttccctttatcaagattatctccaatactcagaaggccaaggttggaggattagtgcatgtttgaagctagtctgagcatcatagtgagcactaggccagccagggctgcatagcaagatcatgtctcaaaataaaacaatatatagagagggctggagagatagatggctcagcagtttcaagcacttgctcttgcagacgactcgggtttagttcctagcattaacacgctggctcacaagtgcatagttttgtttttgtttttgttttttaaataataaagtaaaataagataaaacaaagcaaaaaaaaaaaaaaaaaaaacacatcagaaatggacaaaacaaaacaaaatcaggagggaaaagagcccaggagaagacacaagaatcggaaactcattcattcacacactcaggagtcccacaaaaacaccaaactggaaactataatgtataggcagagggtctggggagggcctggcaggctccgtgcatactgccccagtcttggtgagattgtctgagctttgataatgttgatttagagggccttattttcttgattttctccatcccctctggctcccagtctccttctgcctcttcttcatcggaaaagggatttgaaggagacaccccctaggtttcttagtctctcactctctgtgtacagtctaggagtgggtctttgtatttgttcccatcagctgcaggaggaagcgtctgtaatgatggctgaacaaggcactggtctgtgaggtattaggagtcattttagccttaccttttttccccttaaggctggttctacccttgccctctgggctatctagccgcaggttcttggtcactgaagcaatattgggtatgggttctgtcttgtggagtgggccttaagtcacacagatattggttggttactcctgcaagctctgtgccaccacagcactagcagatctagaggcaggacaccactgtagatcaaagggtttgtggttgggttggtgtttatgtttctctagcatgcagaaaacctttctgtaccaaagaccctagaatatagaaggctctatgtaggcataagtttgacttctccatgttcagtgagtctcacaactgcttataactccagagtaaacttataattccaggaatctgacattctctgctggctttcatgggcaccaggaatgcaccatgatgcactaacatacatccaagcactcatataaaataaataaataaataaataaataaataatcttttaaaaagcagagagagaaagaggagagagatggagagagggagggagggagggagataggtgcacacctgtgcacacacacacacacacacacacacacacacacacacacaaagttgggggcgggggggggaaaccaccttggactatcctgagcttgtttcctcagtggcatggggctgtcgttctgtcagcagagtgaggtcaggatcagctgtgtgatagagaacaggacgtccctttccttcagttggcaccactgtcttcgttctggaatgaagttgcctgtatgctccccagggcccctctgcaacgtggagatcaatgagtgtgcatccagcccatgtggagagggtggctcctgtgtggatggggaaaatggcttccactgcctctgtccacctggctccctgcctccactttgcctacctgcgaaccatccctgtgcccacaagccctgtagtcatggagtctgccatgatgcaccaggcgggtgaggccctttcccaactcccgacccctcttctgctgtctccagccacctgtcacacctcactgcctcccccaccaggttccgctgtgtttgtgagcccgggtggagtggccctcgctgtagccagagcctggctccagatgcctgtgagtcccagccctgccaggctggtggcacctgcaccagtgatggaataggctttcgctgcacctgtgcccctggattccagggtgtgtgaccccatattcctcccccagggcacccgacacccttgtttcttatgtttctttcctgctttttttgtttttaaagctttgcttagtaccttttgttctgtgtgtctctgttacatatgtctgtgtgtggagaccagaaccagaagaaggcatgtgtcgtcttttatcactctctacccattcctctgaggtgtgcagggtctctccctgaccctggggtttgtgctttctcggataggctggaagctactgagtccctgggatgcccctgtttttctttcaacttgcagctggggttacttgttatagaggtgggagggtctgagttctggtcctcatgattgggcctgaggtgctcttaactgctgaaccatccttccagccccatctgcatttccttccttccttccttccttccttgcttgcttgcttccttccttccttcttcattcctttaatttctctcttttttgtcttttctaaatatcagggtggggactccagggatgggggaattgggagcagtgagtttcaaaactatctaaactatctgcttctaacaggccatcagtgtgaggtgctgtccccctgtactccaagcctctgtgagcacggaggccactgtgagtctgaccctgaccggctgactgtctgttcctgtcccccaggctggcaaggtacactaatatcctcctcttcttctcgtctcccttctctcttctttctcttcctcttcctcttcccctctttctcctcttctgcactttgctccatgttgggcaatgccagggagcccagagaggactcagtcctgccctgcctttgaagttgtttctttctgggaaaagacagctggatccagacattcacagcccaggagtcagctcaggagaagagggaagccatatggagctgaggacactgggatacctgagatttgatgacatttttgatcgggtgacttcagagtgtgtattcaacatctaaagagataggcagaatatatttcaggagtggcatgtgccaaacgcccagggatagcggctggtctttgctttccttgactccaccagcgggttcttgagctgcaggcatcctcagacccctttttacccctgtaacctcaattgcttcccctctcacctccaggcccacgatgccagcaggatgtggatgaatgtgccggtgcctcaccctgcggcccccatggtacctgcaccaacctgccagggaatttcaggtgcatctgccacaggggatacactggccccttctgtgatcaagacattgacgactgtgaccccagtaagtgcagggatctttggggcgcttccttccccagggaacccacccatcaagtcataccatgtcctggcactgtgttgctgtttcctgactctcctgacaactattactcccctcattcatgagggtcttacttccatcccagcaccactgtagaaatgggcaatgggctgctgggatgactctgcagggagaggcactgcctctaaacctgatgaacctaggtcagtcctacaagttgtcttctgatctctacatgcctgctatgcacacacacacacacacacacacacacacacacacacacacacacacagagagagaaagagggggcgggagagagagaaaaaaataacaataataacccaaaatagaataaaaagttaaaaatatgttttatttaagccaatatagaaaatattttcatttcacatataagccttcaaaaatttaaattttgctaaatgtattttacatttgcagcttgtctcatttggcctggcagtgagatcacgggcctattaacctcatgtggctagtagtagctacactggtcaccacaggctgtgctgagcgtatgaatcagagcaggcagtggcactacaagtattccttggttctttgaggttgttcccacagcctccatggattacagactctatgggtgtttaagtcccttattgtcaaatggcttaatgtttgcaaatagcctctgcatctcctcccagattatttaaatcatctccaaataactttttattttaaaatgtttacatttacctgttattgtgtgcatgtgtgtgagcgtgtatgtaccacgtgtggaggtcataggacagtatagtccttctaccttgtgggatctggtgttccaactcagggtgttgggtttggtggctttactcaggctgagtcctatcatcaacccagacttcatttttaattgaactggaaggagggaggggggagggagggagggaaggtgagagagaaggggggggtgtggaatgcaaacatgctacattagattgtggaggccagaggacagcttgcaggagttgattttcttcttccaccatgagggttgcagggattgagctcagacaggcagtcctgggtgggcaagcacctttacctactaaagtcatcttgccggtccctctagatggcttgacacaccaagtacgatggacatagcatgtatatgtttgctatactctattgtttagaaaataacaagaaaatagtgtgcatgttctgcccttgggtaatctctgattttccctattgtccatctactggttgaacccacagattctgaacttcagaataggtagggccagttctatagctcaaagggttttgtttttgtttttgtttttgtttttgttttttgtttttcgagacagggtttctctgtatagtcctggctgtcctggaactcactttgtagaccaggctggcctcaaactcagaaattcgcctgcctctgcctcccgagtgctgggattaaaggcgtgcgccaccatgcccggcactcaaaggggctttttttaaaaggatatttttaaaatgtatttatttgtctatgtgttgcatgacacatttgtctacgtgagccatgacattcacgtggtggtcagaggacatcttatagaggggggttgaccacaaaaggggactaactttgatctctgaccaagagtgtctcttgtagtctgggatacacacacgtttaatttctgcacttagaaggcagagtgcaggctcatctctgaactattcaaggccagcctcatctacatattgaactccaggctagtcttggctacatagtaaaactatttcaaaaacaagcaagcaaacaaaaaggactgtcccctctggcttcttcctgagtcttcctgtgtgtaaagcatagctgagtcaggccaggctgatgtgggcataggcactgaccagattattttcctgctcactgcagacccgtgcctccatggtggctcctgccaggatggcgtgggctccttttcctgttcttgcctcgacggctttgctggtcctcgctgtgcccgagatgtggacgaatgtctgagcagcccctgtggccctggcacctgtactgatcacgtggcctccttcacctgtgcctgtccacctggttatggaggcttccactgtgagattgacttgccggactgcagccccaggtgggtggagcatgggctggagactcaggggccagagagggcatcctggactcggcatctgttagagggctggaatgatgctggcacatggctgaggaatgggcaaggctgcttggaagtcacagactccagttctttggaggcctagactgaggcagcgtccgtaggcgaaggagccaggttagatcttcatagtgctatggcttgttgaggatagcaagggtccgaaattgggaagtacttagttctagaaggattggggtggtctttaaggtcttgaacttcttgtcctgttctccagttcctgcttcaatggagggacctgtgtggatggcgtgagctccttcagctgtctgtgtcgccccggctacacaggcacacactgccaatacgaggctgacccctgcttttcccggccctgtctgcacgggggcatctgcaaccccacccacccaggatttgaatgcacctgccgggagggcttcactgggagtcagtgtcaggtgggtggtgtctgaggtccttggtggaagagtccagaaatgaggggggacccgtggggggcatcctgaagggataaggccatctggtttctagggtctctcccagcactgatcttgaagatttcttttgcagaacccagtggactggtgcagccaggcaccctgtcagaatgggggtcgctgtgtccagactggggcttactgcatttgtccacctggatggagtggccgcctgtgcgacatacaaagcctgccctgcacggaggccgcagcccagatgggtgagggaagcatgtggtgcgtgcgtgtggggctgaagggtggtggtgcatccctcttgctggcatgagccaaatgagagcgccatacaacatatgggactaatgaggtgtgtggctcagtatgtgtgtgactggaataactggccagagtgtgactatatctgtcacagtgagacagctgggtgtgtgtgtgactaagctgatagagtcatcaaagtggtgctgtggaaagagaccaggttagctgacaagtggcctgacagcttttggatgtgggtgagacactagggattgatggcagtggggatgttagatgaatgtgtgatgtggccggaataggaaaggtggcatggccacctctgagtctgatgtcaccctctgcttttcaggggtgaggttggagcagctgtgtcaggaaggtggaaagtgcatagacaagggccgctcccactactgtgtgtgtccagagggccgtacgggtagtcactgtgaacacgaggtggatccctgcacggcccagccttgccagcacgggggcacttgccgtggttacatggggggctatgtgtgtgaggtaagtgcgtctcagggagagggaagagaagtcagtcatgcttgcctgtgtttctgtgtcctggtgtgggtccttcccctcccccgtcggtgggagagcagggatgtttcatgtgtagtaggtaagcactctgtatcactcagcttcatccagagtcagcatggctgctatagaatttttttattttattttatttttttttttttttggttagatttttgagacatggtctcgttatgaagctctggctgtcctggaatttgctatgtagtccaagctggcttccaagtcacagcaatccttctgcctctggctctatatgagtgctagataacagtcatgcgccataatacttgtctgcgtgtcttttctttcctccttttctttatcttttcctttcccttctttccttctctttctttctttctttctttctttctttctttctttctttctttctttctttctttctttctttctttctattttgagacagagtctcactatgtagctctggtgggcttaaactattagagatctacctacctcagcctcgtgggtgctaggattaaaggaatgagcaaccaggcctggcctagcagtccttttatgggtatatgtctgtgtgtgtgtgtgcacaggagtgtaaatgcagaggtcaggggcagacatcaggtgtcccctctatcactgtactttgttcgcctgcggttctctcactgaacctcaagttaggctgtagatggtgagccccagtgatcctcctgcctcccccaccccacacctgggtgacacacatagaagaccacacctagctttttaagtaggtactgaggatttgaactcaaatcttcacgtgtgtgcagcaagcgctcttacccactgaaccatctctgcagctccttaaccctcatgcacttgtggatggacttggggcatgtgagtttctgtctccagttctgtgtctctctctgggtatgagtgacttaccattgtgtctggacatgtggtctggggtacctgggtatttttcctgcgtgttctcatcagcctatctccctctgtactgagtgtgctggtggccttcccagtctcaccctgaccaatagcaacttgagggaggagaggatttacttcagcttacaggttactgtccatcattgagggaggctgtgaaagaagcccaaggcagggaaccatcgtccagaactgaagcagagaccatagaattgtgttgctttcccggagcggtctgggtcttcccacatcaatcagcactcaagaaaatgcccccacagacatgctataggccaatctgaaggaggcagtttctcaagcaagattccctcttcccagataggtctaggtttggttcaagttcatgcacacacacagataacaagtgtgggcacagtgtgtttccttgtactgggtaagtctgccccctacactggctttgtgttggatgtctatgtgtgtccttttgtggtgagattctgtgtgctcacaggtatgccgaggcacgtctgtgtccctcggggctgagtgaattcctttcttgcctcaatacagtgtccagctggctatgctggtgacagttgtgaggataatatagatgagtgtgcttcccagccctgccagaacggaggctcctgtatcgatcttgtggcccgctatctctgttcctgtccccctggcacactgggtatgttaaggccagggttgggggcaggataagaggatgagtttctagcctccactgaccatgctcctataccctaggagttctctgtgagatcaatgaggacgactgtgacctaggcccatccttggactcaggcgttcagtgcctacacaatggcacctgtgtggacctggtgggtggcttccgctgtaactgtcccccaggatacacaggtctgcactgtgaggcagacatcaatgagtgtcgcccgggtgcctgccatgcagcgcatactcgggactgcctacaagatccaggtgggcatttccgctgcgtctgccatcctggcttcacaggtaagaatggcagagagcctggccagaaacctgatgtggttctgcttctgtagttgatcctcctgcatctgtttgttcagggcctcgctgtcagattgctctgtccccctgtgagtcccagccatgtcagcatggaggccagtgccgtcacagcctaggccgtggaggtgggctgaccttcacctgtcactgtgtcccggtaggtgtgattggtaggggttggaacccttggggaaagaaaaggcctgtggctttagggaagcataggtctatacgggaaaagtagaaggaaaggaggttctgaaattatgaaattatgaaattatggtttggagtgtaacttagtgaaattgtgacttggctttgttgcctcctggggaggtatggcttatcttcaaaatgaggtcagtagaggaaaggttgctggaattggaggggtgggggtggggtgtcagcatttctcaagtcttgacctccattcttttctctcttttccactctcctgtttcttctcactaccaattttttctctttctgtctcctcacttcaccattagccattctggggtctgcgttgtgagcgggtggcacgctcttgccgagagctgcagtgcccagtgggtatcccatgccagcagacagcccgtggaccacgctgcgcttgtcctccggggctgtccgggccctcctgccgggtttctagggcgtcaccctcaggagctactaacgccagctgcgcctctgccccttgtctgcatgggggctcatgcctacctgtacagagtgtccctttcttccgctgtgtgtgcgctccgggctggggcggcccgcgttgtgagaccccttccgcagcccctgaggtccccgaggagccacggtgcccgcgagcggcttgccaggccaagcgaggggaccagaactgcgatcgtgagtgcaacaccccaggctgtggctgggatggcggtgactgctcactgaacgtggacgacccctggaggcagtgtgaggcactgcagtgctggcgtctcttcaacaacagccggtgtgacccggcctgcagctctccagcctgcctctatgacaactttgactgctactctggtggccgcgaccgcacctgcaagtgagccccctgactctgtccttctgtctatctatatgttgcactgtcagtgagccacatctgtcccagtttgtctgtcagtctgtttttggctcgtctgatgggctgtcccttccagctgctaccaccagggcactggtggtttcatccgcctgtttccacccatgtgcctcatctctgcctatttatatttttacctatatatcttgcccatttgcttctgtctgcctgcagttacacatcccatctgtccacacttaggacttgttcatttgtctttcttaaaattttaaaaattaaactgggtctagttgctcacacattttaatgccagcatttcagaggcatagacagcaggtctctgtagggattcaaggccagcatgatctacatagtgagttctaggccagccaaggccacacagtgataccctgtctcaaaacaaaaacaaacaaacaaacaaacaaagcaaaacaggagcccacagcaggtgctcattcaagagcatacaatagtgatactggttgagcatcccaaatcccaaacctgaatccctgagccatgtggggcatgggcatgatgccacagtggaaaattccatcctttgccccaatgataagttacagtcaaaattcagacacaggttggtctgtagtggcagagcaccttcctagatggcatgtggcccttggttcaatccccgcccagcatggaaataaaacaaccaccaaaggcaggcacactaaaaatatatagttacttttgggtatgtgtatttttgtctttagacttgggcctcatcccagaggtgccttatataaattatgtatagagggctggggatgtagctcacttgatggaatgcttgcctaacatccacagagccaggcttgggtccccagcaccacctaaactgtgagaggtgctgtgtgcttgtaatcttggctctcagcaggtggatcaggaggataataagttcaagatcaccctcagcaacatagttttgttttctttgttaaaaataaggcttcagccaggtgtggtggcgcatgcctttaattccagcacttgggaggcagaagcaagtggatatctgagttcaaagccagcctggtctacaaagtgagttccaggacagccagagctacacagagaaaccctgtctcgaaaaaccaaaataaataaataaaataaataaataaataaataataaaaataaaaatagggcctcacaattcctggctggaattggctgtgtaaatcaggctggccctgaacacacagagatatccttgtctctgcctctggagtactggaattaaaggtatatgccagcctgtctgactaacatagggagtttgaggccagcttgagatacatgagatcttatcttaaaatatttatagaaagatatctcattgcatttatttgcatatataatatttaagcatatgtgtaaatattctaaaactataatttgaactctgaaatagttttagtcccaattatttcaaattaggaacacagaacttgtgcttatgcagccaacgactgcatttgcagaaagcatatgcaagaacatttcccaccaccaccatccccaaccccataccagcactgtccactactgctattctctgtgtgtgcatttaggtgacccgaagaatctctgagtttgtgttctctgtccccagccctgtttatgagaagtactgcgccgaccactttgcagatggccgttgtgaccagggctgcaacactgaggaatgcggctgggatgggctggactgtgccagcgaggtcccggcccttttggcccgaggggttctggtcctcacagttcttctgcctcctgaagagttgctgcgctccagtgccgactttctgcagcgactcagcgctattctgcgcacctcactgcgcttccgcttggacgcacgtggccaggccatggtcttcccctatcaccggccaagccctggctctgaatcccgggtccgtcgtgagctgggtcctgaggtgatcgggtgagtgactgtggctcagggctgggtacagcggttagggcacccgtggtccagaccgtctgtttcacgcttcttagttgagagctctcttggcaaggcgtcttccacaggtttttccgtgtctgtcgggttgacatctttgctatggggggggggggggttcatcctctgtacactacagggagcctcgctgcagcgctgagattttactcttcaaatgcaggtgacagcaccttcttgtgtgcatgatgtgtgtgtgtaggtccccaagtgccatgacgacgcatgcctgtagaggttagaggacaacagtgtgaagtcagttctctcttctcatcaggatgtcaggcttgcatgatgagcatgttgcctgtgagccatttcgctggcttgtttgttgttttcttctcgttgtttgctttgtttgtgttttataggcactaacctgaaattcaatctgtagcccaggttggctttgaactcatggttctcctactcagcccttccaagtactaggattgcaggcatacaatatcgcccccgacactcactctctttcctccccactctcttcattcccctttcccctcatctcacaaaagttgtagaacttgctcactggtgacctggtagggggaacctgaagtgggagaggcatttttttgagggtggaccagggcctgaagattgggccctgacaaggaaaggagagctgaactttagagatgctgtggtttgtgggttctgattggatgcaggcagaagtcaagtagatgggtgaggtgacacatggcccctttcttggctaaagtggcttagtgatgaggacatagggctaagcaggggccaccttggaacttgctattcttagggggtcttgaggtatggggaggaaccccaggagatgactgagggctgaagatgcatacaagacaactgggtattggaggccattggagggaaagaagggagaatcaaggaagcaggagagaaggaaatgaagaaggaatctaaggactctcagagacctaagaattgggggacagtagaggacgtggctcagtggtagagaacctaccttgtatattcaaggccccaagttcatctccaataccacaaagaaggataaaggagaaaaagcaagcttttagagaaactgacatggtacactgtcccagcaaagggcgacaccaacagactctaccagggcgaatgaagacattattgaattcacagctttgagacactgtgaccaataagatctgttgggagagacagtggagttggatgccagctgagagctgggtgggtggtagagacatggagagtggagacggatgtggctctctgaggtcgggtgtctaggatgctgtagacaagtgttgagcctttgggtccctctgctctgttcccacagctctgtggtgatgctggagattgacaaccggctctgtctgcagtcagctgagaatgaccactgcttccctgatgcccagagtgctgctgactacctgggagccttgtcagcagtggagcgacttgatttcccatacccacttcgggatgtgcgaggtgagctgggaagaagagagggtagtacattagagcgtgtagccccagagatggttgaatcctatagtatggttgaagccctgtgagtaaagccccatcttctggctgaagctgtcccatagctaagccaaccccatgggtaagagccacttaaaattgaaaccttacttgtagtcctgctccacggctacagccctgcttatagctgagtatcgcccatggctgaaatctgctcactcgttcctgccctgatacttggctgaaggctcatctgctgcttccatcctacaggagagccgctggaggccccagagcagagcgtgccactgctgccactgctggtggcaggggctgtctttctactcatcatcttcatcctgggtgtcatggttgccaggcgaaagcgagaacacagcaccctctggttccctgagggttttgcattacacaaggacatagctgctggccacaagggccggagggagcctgtgggacaagatgcactgggaatgaagtaagaacctcacatgctctacatccccaactgtgggtcccttgtaagctctagaccatactcacctcggtcatattccaacctctgaccccagcctaaccttaactacagactccatctgggccttcagtgttaatcctcttgacctatgaccccatgatccctgaggattgccccaacccctatcccttgacgtgattttctatttctatacatttcctgactcatatctttccctgacgccatcctgaactaacctcacaaaattcattcttatgactgctaacctcaagacttttgtcatttcaacctgtccctgaccacgactgtatccttgatgactcctgaatcatctctggcttcaacccacctgtacctttgacctcacctcagacccctgtttctacccaatcctgtgttaattcccttgtgatcctcaatcccaagccttcaacttgacttattcctcaacaccaatagccatattctgaccttaacccttccctatgacaccataactcctgactcctcatcctgatgtttcaagcctggctatcccgtttgtcacaagagagtaactcctcttccatgagctttgataactcctttatcaaagttatgagaggaaacttcctttgcccctgcagtttgtctcatctatcctcatgcatccatgttccattgtgtgtgccactcttgactctgagttcaccttatgagtctggccacctcagatgtgaggtgtggagcaggtataaagaccgttgtttggatccctagaacctgtataaatgccataaaatccttaagtgctcagtgggcccggcagcctgacttggaagatggagtcctggtccctagagcaagctgactagtgagtctagtcatatccgtgagctcttggtttgattgagggacccttcctcgatgaccaagactgaggagagattcctctttatcaacctcagacctgtgcacccaaaacacacacctgcactcacagatggaaagagaaaacattttgattgccacctgacatgcttcctgcaacttcatctctatcctcctatccatcattggaatctttgtcaacgacccaggacccctcctcagttcctgaccccttacctacctctatgacctctgcctgaaccaaaccctaaaccctcttcccaggccagagttctctagtgtactttctcacgtctttttcttcttgttttccccaaggaacatggccaagggtgagagtctgatgggggaggtggtcacagacttgaatgactcagaatgtccagaggccaagagactgaaggtattaacctgcttctctgactctttccttcagggttccaagttgggatcccctaacagctggagagcccagggaagtctctcttctcccgattgcctcagccccagacaatttccacatctgtgtggacctcacttttcccttaatgtgattctgttgctgtgttgggaggaacagagatttaccatgggttccctgggtgggtggggcttccccttcttcatatggttcctcctcacacaggttcctgctcccaggtctgcctagggtcaccctagagcagcagctcttaacctgtgggttgtgaacctatttgggctgcgctcttcagtttcagatggctcagtcggtaaagaaagaaaagaaagccaggcgtcttgtggtacacaggtagtcccagtgaacaacctttccacagaggttacctaagaccattgaaaaacacaggtttttacattatgattcataatagtagcaaaattacagttatgaagtagcagtgaaaataattttatggctggggggtcatcacaacatgaggaactatattaaagggtggcagcattaagaagagtgagaaccaatgccctagagggaacaccctagtccacagagtgattgttttctgttccttgtctagcatccccagcctgaaagtgtccccagctgctctagggttccatctatctttaggaaccacacccatttatttgggctgtgctcttcagtttcagatggctcagtcgctaaagaaaaaaaaaaaaaaagaaagccaggcgcttgtggtacacaggtagtcccagtgctggtgaggtggcaagagcaaactcctggctggttaactagcctagccttcttggcaagttcttagaagaaccctgtctcataaaggaagatggatcgtgccagagagtggtggcacacacctttaaccccagcactcgggaggcagaggcaggtgtttctctgagtttgaggccagcctggtctacaaagtgagttccagtacaaccagggctacacagagaaaccctgtctcaaaaaatgacacaataacaacagtaacaaaaagagggacagtgctacagagcaatgacataggaggcggttctttggcttccatacacacagacacactcgtgtgcacacacacacaagcttgcacactcatgcaacctgagggctgggtaatgtatgaaggaaaggtctttctgcctcagcttctccagtagctgggactgcaggtttgagtcacaacatccagcctccatatttcttttaccagggatctgaagtatccagggttggaggactacatttaattttttaaaatttcaaatattttgctctttttatttatttatatttatttttaaaaattggagactagagagatgactcaacagttaaatgctggctgctcttccagaggacctgtgtttgattcccagtacccacacagtggcttacaaatgtctgtaacttcagttcctatagatgtttgacgtcttctggtctcccaccaggcatgcatgacaggagtgtgccaccgtgacagctgtagtttgttaatttaaaaatttgcattcacttattatttatttattttgtgtgtgggccgtgtgagctacattatgggggagggaggtcagagaacaactgtgggcgttattctctttccactgtgtgaattccagagatcaaactcaggtcattgggcttggtgacaagttatctcttgttttgataatttaaataaaaatatcctttttcttttaaaaaaatattaatttgtttttttttttgagacaggatttctctgtgtagccttggctgcccaggaactcactctgtagaccaagctggcctcgaactcagaaatccgcctgcctctgcctcccaagtgctgggattaaaggcgtgcgccaccaccacccggcttagatctacctatttctgtctcttgagtcctgggattaaagatgtcccccactgccacccagctaaatatttatttttgtgtatatgtgtacacattggtgtgtgtgccatggcaatgtgtggagaagcacatttaatctgcaagtacgtttacctgctgagcactctccccaactctgttttatttttttgagacagcaggttgcttttggtctcctacacctgatacagctttatcagcagcactggtgggaatgcccacccttttttgttactgcctttcccacagcaaatgagaggtgagcgcctgtatctgtgtccctccaggtagaggagccgggcatgggtgcagaggagcctgaggactgtcgccagtggacccaacaccacctggttgctgctgatatccgtgtggcaccagccacagcgctgactcctcctcagggagatgcagatgcagacggagtggatgtcaacgtccgagggcctggtgagtgccctcccaaagaggccctcattggtcctacctgctggatcccatgcaggggttctgggagcgtctgggcctctgggcctctgggcctctgggcctctgggcctgctgcgtgtgttgtattctaagtgattggaacgccatgcaaggtgaagcagaaggtgggctggctgcaactgagaaccctgaggctgtgtggaccctggactctttcttttatgagattaaaatttccttcttttcttttcttttcttttcttttcttttcttttctttatttttcttttctttttttctttatttttttctttttttgtttttctttttttagatttatttattattatatctaagtacactgttgctctcctcagacacaccagaagagggtgtcagatctcattatgagtggttgtgagccaccatgtggttgctgggatttgaactcaggaccttcagaagagcagtcagtgcccttaccagctgagccatctcaccagccctctctttttgtttttcaagacagggttctctgtgtagccctggctgtcctggaactcactctgtaaaccaagctggcctctgaactctgagatccacctgcctctgcctcttgagtgctggggttgaaggtgtttgctcccccaaccccagctctttcttcctgtgtagccctggctgtcctggaacttactctgtagaccaggctggctccaaacccatagagctccacttacctcatcctcccgagtactgggattaaaggcttgttccagcaccaccactgcctggctcttgctcttgctctttctctctttctctctttctctctttctctctttctctctttctttctctttctctttctttccctttctttctctctctctctctctttctccttcttcttcctcctcctctgcctcctcttcctcttccctctcctcttcttcctcttcctcttcctcctcctctttcttgttatacttttatataagatttcactatatagctctggctatcctggaactcataatgtagatcaagttgttgtgaagtcacagagatcctcctgcctctgtcccccaagtgctggaattaaatgtgtatgccatcatatccagctgggatttatttaaaaatcacacttatttgtgtgtattcgtgtactcggaagtcagaggacaccttactggagttgattatctcctatctcggtccaagagatctcattcacgctgtccagtttctctgtagcttctctatccatcaagctcctggatgttcaatccctccttacccctctagcccccttagcttcagccatacagcttgtcaggagagtctcggattagttctgacagggtgaactagaaccacgtacttattcctgggccaatgaatgtggctagggcctataatatgcagataagccagtccccatctggtctaagtagggcttgctattagggaattcgtgaggagaactagggaaaaggtggttcccttcccctttattccaagtgctcggcttcctggaatcgcttttgcggtccatcatgtaatcttcgtggggtggctttgcagttgggagaatttacctctgtctgtggcgctgtgtgtcacagtgcccctgacatttgccccaggaagacccctatgcctgtgacacactactggttcctgcagatggcttcaccccacttatgctggcctccttctgtgggggagccctggagccgatgccagctgaggaggatgaggcggatgacacatcagccagcattatctcagatctgatctgtcaaggggcccagctcggggcacggactgaccgcactggcgagaccgccctgcatttggctgcccgctatgctagagcggatgcagccaagcgtctcctggatgctggggcggacaccaacgcccaggatcattcgggccgcacccccctgcacaccgcagtgacagctgatgcccagggtgtcttccaggtgagacaggcctgtctcttcagactgcagagctgctgggaggggatcagacacacctagattggagccccggtctgtcttgcaaggcttttgtcatttggaaataggaataggtagtatctcacctagatttcccgccggccccccccccccccccccaggacagggtttctgtatagtcctggctgtgctagaactcactccaataaaccaggctggcctcgaactcagaaagatccacttgcctctgcctctggagtgctaggattaaaggcatgcaccactaacactggatagaatttttttcttttttaattttattaatatatgtaagtacactgtagctgtcttcagacactccagaagagggagtcagaacttgttgcagatggttgtaagccaccatgtggttgctgggatttgaactctggaccttcggaagagcagtcgggtgctcttacccactgagccatctcaccagccctggatagaattttttaaaaagtatcattaaaattacatttatttggtgtgtgtgtgtatgttatgctgattggtttgtgtttgtttgtgtggcatatgtgtacctgggtatgtacattgcccagcttgctgaagctagaggaggctgttgattgttctgctctatcgtgctccacccaatcttttgagacagagcccatcagcgagcctggagttgagctggtgtccagaaagctctcttgatcctcttgtcttcttccccacagccctggggcacacatgaccaagactggctttttaggtggactttgggctctgacgtcaggtgcttgtcccctgagccatctccctgtccttccatatcatactgttacttaaatccattttgaatgagcatattttttgatttataatgtgcttagcaggaatagcacctcattttaagtcaggagatatctgtagctcctgggttccaaagctgtggcatttggggttcagggtgtggtatactcctcccttggcataatgtcctgccatggcttttgtcgtcgttagattctcatcaggaaccgctccactgacctggatgcccgaatggcagatggctctactgcactgatcctggcagcccgcctggcagtggagggcatggtggaagagctcatcgccagccatgccgatgtcaatgcagtggatgagcttggtaagtgctgcggaggggatggggaggggctgtggtgccactgccctcttaatgtgaagtcacacctacgttgacagcaaggtgtgcacgccagggctcccaaacggatggaacaggacccacagagtgttctggttcagaaaagatgtagactcccttctcctcccctcccttccctttttcttccactcccttcctttcccctcccccatctcctagaccccatcccccactttcacattatgatactcctcttcctccccttcctcccgggcttccttctagagtccaaatcgttgatcaaacccaaagtgcttgtctttgagtctgacttatttcacttactgtgatgatctccagttctattcatttcttgaagattgtttaatttggctcccccgacttttgagacagggtctcatgcaagctaggctcgcctccaacttactgtgtagtcaacgaccttgaaatctggaccctcctgcctctgtttcccaagttaggcattcgtcatcacatccagcccaatttctttctttgttttggttgagtagcattctgttgtatgcagtgtgggtgggcacctaggctgagcaactccattgtatgcagtgtgggtgggcacttaggctgactccatagtgtaccggcttcgaatagcaatgtggatgccacagttcgcatgggcatgcagacatctcttattgtatgctgactcagccctcaagtatagacccaggggcacaggagtgccatacctgaacctagggtagttctttttttttttttttaagaattatttatttattatatgcaagtacactgtagctgtcttcacacactctagaagagcgcgtcagatctcattacggatggttgtgagccatcatgtggttgctaggatttgaactcaggacctttggaagagcagttactgctcttaaccactgagccatctctccagccccctagggtagttctacatttagttttttgaggaccctccatagcattctctatacttactacttagcatacttagttactttatcattaatagggcatgaagatccctttctctagtgtttgtattctttctttctttttcccttttttggctgttttcatgattgaagccattctggtagcatctaagtacagtttttgtctgtttttatttttttgttttgttttgttttcagacagggttactctgtgtagccctggctggccatggaactcacactgtagaccaggctggccttgaactcagaaatctgcctgcctctgcctcctgagtgctgggatcaaaggccgggatcaccaccacgcccggctgagaccctctttttctgccaggcacagaaaggccaccttttgccttcctgcccagctataggctattcagctctttatttaaccaatcaggagacgatggagaacattgttttacaaaataccaagttagaccaaagtctggactgtaaccagatctctgggaacagaaatcagcatctgaatacacagtgcacaaaaccaccccccaacgggacttcatcgccaactgggggtaaacgctcatgctttgcccacactgaccaagcacacgagcttgcttcttagtgtgctgtctagatgctttgttaaggaaacggactctcctgctacctgctagacagtgggataaacttgcatgtggtgatctgtgggggcagctgcctggtagtggcctgcttggcttgggtatgtcatgagcaagggtcacagcaagggttcttgtattcacaggccgcacagctgtagacactctaagacacttacacacatttgtttgacgtacccttggttctcaacttcatcagcattgacatctatggttgggtaattcttggtctgcgtgttggatgaaggtctgggctgtgcactccggcctgtggggcagcatcccaccaggagcatcccatctctacaatgttactgagtatagtgggcttcttccctgggtgggcctttgtttcaacaacaaaaggatttagaaatggtctcagaaggaagctcaaacacagttagactagagtttgatagcaaagcaacagatcaagcaagctgtcagtctctctggaatagagagatggagaagatgcattcatgtcttttgagctgtggctggaaagccgcagcctggtgggagctgggggagaggtgagcttccgaggacaagtgcgagctcatgtcctggctttagccaggatctgaaagaaaaggaattgagaagaaagaaaaggggtgcggtggccaatgcatttgctatgccagggcgaagacctgagtttggatcttcagaaacctatgtaaatgctaagtgggtgtgatggccacctgtaattccagacacacacacacacacacacacacacacacacacataccccatgtatacacagaaaaagaaaaaaatattcagaaaagctggcagggtgcataatggtggctccaatgtgctgcctgcctgatggatgtcctgcaagtagctgctttgagggagaggctcctaggaagggaaaatacaatatgtcattaaggggataattagtccttccctgtcaatagcatgtctttaggtgactcagagtttttggcgggacaaaggcagactcttggacagataattgacagactctattgggatacctttaaaaatagtctatttatgtttactttatgtgtagggtgtttcgcctgcatgtatgtctgtgcaccccgtgcatgcagtgcccacacaggccagaagaggagagggcatcagatcctctggaactggagttacagaccgttgtgaactgttatgtgggtgctgggaactgaacctgggtcttctagaagatcagccagtactcttaaccactgaaccatctcccagctccccgaattaactcttaaaaggagacagtcataagtagcatgttgggttccaccaaggattggaactagaactcagattctatttaagaagcagctttgcattaagtgggcctcaacaaactcagacactcccagctttttatggcaagttgatgtctctcctaagtagtcttagtcctgtaagactactgtaacaggcaaatgacctgggggtgggggtggggggagggcagtcattcatggttgatattagctaacttttaaaaggcaacaatagaggggccaaaggtagagagaaaaagacttgattgtggatgccaaaatgcctacaggggaagatgggatgtgcctcttataagggaggactcctgtagtccacaaggaaagctgggaagtgtagtccttcagcaggaaactcggtcccaattctgagaggatattaaaaagacgcagggcaggatgtcatagctttttcttttctttccccggaggagagtctttatcagaggggttagttctgggtccagtaaagggaaactctctggccatgttgtatgctagagtcttgattagctgtgatggatcaggccattgtcattccgcattcctgaggagagctgggaaaatatttattagaaggccacaagcagatgatttggttatagagtagagctgagagattttctgggaaggttgagggagggggaggagcaggagcctaggcagaagagacaagagtgagacaaagtgggcagaggctggtcccagggtgacagtgactggtcccattgccaggactgctagaaaaatgagctagcagaagcagcagagggctacaaagcagcagaggacgggaagaggggtcaataaagggttaatgtgaagaactgtatctggcctgtggtggaagagatcacggtcccttttctattcagcacctaaacaattgttgtttaggtgctgaatagaaattgttgttgtttctcagatgtgtcaggaacaaatggagctgttgtgggatgagagccactgagagagctcgaatgggaaattctgcgtgggttcagtaagcaaaacatttaaagacacggtaaaaatgtaggtattaagataaagaagggagaaagcgtgaggcaggggaagagacagagtggggggaagccaggaggaacttttgtctggtccctgtgcctttaggccccatttcaaaccaggcagcatttgttggcgtggtagcagccctgatcaaggagggcagcagtcagagctagtccttttgagttgtagagtaccgcgtccgtcaaggagttgacctgctcctagagggtgtcacgctgatctgagctggtgacacagagttcactgatgcagcgaaggcttcagcccagctgtgacacagagaagttcactgaaggtgtcagtccagctgtttcagtccagagtggcaagaagaggggtggcttggctcattcatggtccatcatcccctcttagatgtcgtccaaactgctcttaggggcctggggtgttggtctccctgtgacttctttaaggctggcagaagtgggacgaggggagcagccgtcagagtgcctcagaagagggtccatccaaggggacatgatagaacttaagagttggcaaagatccagaacataagtcccagggtgataacacacagctgaatggagacaaagttacagaaggaaaactttgacaaccagttgaatacaactggacaactgactgcaccagaaggatacggtagctgtaagtagctctttgtgtgcattagaatcctgccagctaagaatgactgtggctcagtgctggacactagagctttctctgaggattggaaggaatatttgttggcccagtctgttttggctgctactggcctcttgtggctgatggccacttgaaaactggcttggaccaatgaagagctgttttctttagttcatccaatgtttacaatattgggccagggatatagctcagttggtgaagtaatcctgctaggtccattctctactaccacataaaatcaggcgtgttaatcttaggcaggcccaggatcccagggctcaggacccatctgcagccaggaggatcagaagttcgtcttcaggtgcacagtgagttacaggccagcctaggatacttgagtccttgtgtttgttttgtttttaactatttaaaaaaatatttattttatgtgtatgagtataccatagctgtcctcaaacacaccagaagagggcattacagatgtttgtgagccaccatgtggttcctgggaattgaactcaggacctctggcagaggataaccttttaaccgctgagccatttctccagcccctgagaccttgtctcaacaacaacaacaacaacaacaacaacaacaacaacaacagttaaacgcacatagtgttcttgcagaggacctgacaggggttcttaccacctaagttgggctccccacaaccatctgtaactctagttccagggaatctgatgccctcttctggacgctgcagatacctgcacccatatgcacctcccctgtgcaaacacacatatacacataattaaaaactataaaaataaatacatcttaaaaggaaaagcccaccctgaacaggcaatctagaatataaagactgaaatcatatcgacaactgcctaaagcttgggatgggaggagactgatcaatgatgagtaattaaagtgatggaaatgttgtaaaacctgattaattgtaaattatgactcaacaaaaatatagtcttgtatatatacaagaaaagatattaaaaacaaaaattttgagacatcgtcttagcgttaactgtcaacttgacatagcctagagtctctgagaagtgtgtctcggatcagtgtgattgtcttgattgttaactgatataggagggttcagcccactgtgggcagcagtgttctgggccgtgtgatcctgagctatacaggaaggttagctaaggatgaacctgcaagtgagctggcggcaccaccatcctgcacggtttctgctgtgctttggctgggaagtgagctccttagttggagctaatgctgtcaaacctgccttcaggttattgccttacttcactcagtgatggattgtgatctaagagtgcaagccaaataggtcccaagtgctgtgtgtgccaccacaacggaggtgctagggattaaacccacacctccgtgcatgccaggcaagcacgctactgacttatcgacaagcccagcacatgttctaaatattctatattttaaaaagctttattatataaaaaaccatgcaagtaaatgaaagtaccacatatcatgctggagacatggctcagtagttaagagcactaactgctcttagagaggacctgagttcaggtcccagcatacacagagtggtttataacttcctgtagctccagatccagaaagacatgatgtcccctctggcctccttgggcactacagtcacatgcacatacccccacacgtacatataaataataaaagaaaaaattttaagggttggagagatggctcagcagttaagaatactgtctgctcttccagaggtcctgagttcaatccctaggaaccacatggtggctcacagccatctgtaatgggatctgatgccctcttctggcacgcaggtgtacatgcagatagagcactcatatataaaataaatattttaaaaaatcatatatccaaattaaccagacccaaagtctatataacgtaataaatttatatacagttagaattggccttctggcagaagctatgaaacttcacattaaattttatatttttagtactgaacaaattttagattcttttttttttttttcttttttctttttgttttttcgagacagagtttctctgtgtagccctggctgtcgtggaacacactttgtagaccaggctggcctcgaactcagaaatccgcctgcctctgcctcccgagtgctgggattaaaggcgtgtgccaccacgcccggctattttaggttcttaggttcttatccaccaccaaccccaaaagaccatggcaaatcagaactagaacacctaagtttttgtcgtcattcacaaatgcctcgtgcctgcacttagtctttcctcctcccatacatttagcctttcttgttctcttctctctgtgttgtgctgtacccacatgaacatgtgtggctttagcttgcatttccctcatgggaaatgaacctggctaacttttcacctgatcattggtcgtacgtagatcttccttgaggaaataattatttggaatttattttggtggtgctggagatcaaacgcaggctcttctagatgctactcagcgttccagggctcagctgcacacctaagcaccgagattatcatttctcactgttattagacttcatatatatgttagaattgtatatgtgagccggcagtggtgacgcacgcctttaatcccagcacttgggaggcagaggcaggtggatttctgagttcaaggccagcttggtctacagagtgagttccaggatagccaggactgcacagagaaaccctgtctcgaaaaaccaattaaaaaaaaaagaattgtgtgtgtgtgtgtgtgtgtgtgtgtgtgtgtgtgtgtgtgtgtgtacacatacacagatcccattacagatggttgtgagccaccatgtggttgctgggatttgaactcaggacctctggaagagcagtcagtgctcttaactgctgagccacctctctagtccttgccaggccattttctacacatttctgcctgttctgtaatggagaacctgacatgagcctgacactgatgtttatctccttacagggaaatctgccttacactgggctgcagctgtgaacaacgtggaggctaccttggctctgctgaaaaatggagccaacaaggacatgcaggacagcaaggtgagccactgggggcctacagtgctacagcccctgaatgtgacaaagcagagtcagggaggaataagcctgttaggggcatccatcttgcttgcagtcagctctcaggagtgggattctggcctgaaggctgtgagagcaaactgttgaagtcgctgtgtgacatcgaacgcagttgtccccattgtgacaggggaagcagcagcatgagctgctgagcttgaacaagcctacttttcttccttccttctttctttctttttgagactaacttcctacctcaggttggccttgaactcacaattctcatatttctactaactaaatgctggtatatgctctcatgcctggctttttcttttttcaatgatatattttatttttaaatagtgtgtgcgtgtaagtatgtggacatgagttgcagatatccttgaagcaagaagagggtgttgaatccccctagagctgaatctataggcagttgtgaactgcccgtgtggataccagggatggcactgaagtcaccaggctgagtgacaagtgtggttacccactgtgccgtcttgccgtcttgccagtctttacattgtttccttctcttttggagatcatgtcttactggttaatttagcaaaagctgacctcaaactcttggtgagtcttctgccttagcctctcaagtgctggggtcataggctcaggttctctctctgtctgtctccatctctgtctttccctcccttccttccttccttccttccttccttccttccttccttccttccttccttctttctttctttctttctttctttctttctttctttctttctttctttctttctttctttctttctttctaagacaaggactcactttgtagaccaggctagcctagaactcagagacccttttgcctttgtgtcctggatggtaggactgaaggtgtacaggaccaccagaaccccaacaactcactttttaatcttttgtttttttggtttttcaagatagggtttctctgtatagtcctggctgtcctggacctcactttgtagaccaggacaaagaaactggtagaactcagaaatctgcctgcctctgcctcccgagtgctgggattaaaggcgtgctttttaatcttttataactctttttaaaacttttataacatcaccatgcagtttattttgatgactggaagttttgatgcccctttaaattttgtgcctaaggtaagtcctggccagatgtgcctgctacagagagagcttgtttcttcaacagagctggaatccaaactcaggtccatctatttccagggtatcaactcttagttctcaggctgccctagagattagaaagctaggtgttggccagagtgactgacctccgtgggttctgcccctcccccgcctcaggaagagacgccgctgttcttggccgctcgggagggcagctatgaggctgccaagctgctgctggatcatctcgccaaccgggagatcacagatcacttggacaggctgccccgggacgtggcccaggagcggctgcaccaggacattgtgcggttgctggaccagcccagtggacctcgaagtccctctggtccccatggcttagggccattgctctgcccaccaggggccttccttcctggcctcaaagcggtgcagtctgggaccaagaagagcaggaggccacctggcaagaccgggctggggccacagggaactcgtggtcggggcaagaagctgacactagcctgtccaggacctctggcagacagctctgtcacactgtcaccggtggactctctggactcaccacggcctttcagtgggccccctgcttcccctggaggcttccccttggagggcccctatgccaccacggccaccgcggtgtccttggcacagctaggcgcaagtagggcgggtcctctggggcgccagcctcctgggggctgtgtgctcagctttggtctgctcaatcctgtagctgttcccctcgactgggccaggctgcctccacctgcccctccagggccctcattcctgctgcccctggctccgggaccccagttgctcaacccaggagccccagtttctccccaagagcggcccccaccctacctggctgctccaggacatggagaggaatatcctgcagcaggaacccgcagtagccccaccaaggcccgcttcctgcgggttcccagcgagcatccttatttgaccccgtctcctgagtccccagagcactgggccagcccatcccccccatccctctcagactggtctgactcaacacctagcccagcaactgctaccaatgccacagcctctggagccctgcctgctcagccacaccccatatctgttccctccctccctcagtcccagactcagctgggaccccaaccagaagttacccccaagaggcaggtgatggcctaagttcttggatttgaggggtgctgaagtgacactcctctatgacttctttcttcctcctttttaatcttactctcatccctttctctctgtcccagccttcctgcacctctctgtcttgtagtgtgaccaagttggtcaccagcccagacccccagtcttcctttatttataatgggtaggggctgaccttccaccaccttggccccctaagggatctgggacctccttttgatccctctccctgcctcaacttcctcccccccctctttctgcttctcattgtctcacactctgacaagagtgagttattatttttttcttttttacattttgtatagagacaaattcatttaaacaaacttattattattattattttttacaaaatatatatatggagttgctcccttccccccgctgcaaattcctccagcgcccccgtggggctgagtctgtgggcccgtttggccaatccggactctgtgtactgagtacacagatatgactagggctccacgtactgagtatgtggccctcgtatgtaccaagtagccagccttgggcacaccctcccctggggtcaggggacatttgggagcctccttcccctccccattccccttcctcacttcactgcattccagataagacgtgtagactcactgggaaaggggtcttgtctgctcaaagcctcaactccaggctcacctcccagagcctggctcaccttttagggcctggggtgggggggcacgtcaggggagatgtattttgtatgcattccacttctaattgtaaatacagggcagaaggtgggagtggctctccctcttcctgttgttctcttggctcagcctgcctaatagaaatgtttttaggctgtttttgtaatatggcacctggtcacagtcctttgtagctgaattcccaggtcctgcactgtacaaccctcaccttctcagttcccttaccacctaataaaggaatagttaatacccaagtgtctggtttctgtgcaaggtccaaagtgggggtttctgggcctcttcctccacagggctaacttgaactcccatcttggggcagagccatgtgctttgtcagtccacctttgactcctttttggtcaggtatctagcttcatttctgttgctttaagcaaatacctaacagaaaagaaacttaagggacagagggactttttcctaactaaatattccaggttacagtccaacattgtgaagaagaggcaggaacttaaaatagttcaccacatcacaccacagtcaagagcagagagaaagaagtgagtggatgcatgcttgcttccttgtgtcgggctgaatcattacactcttaaacagtttagaaacccttgtttggggaatgatgccacccacagtaggcagtgtcttcccacattaatttgacttaattacgacaatctcctaaagacattctttcaggtcaactcgatgtagacaacttctgttcccaggtgatttgtaagttctgtcattgtgacaaaactgatcatcatatcaggtggtttcaatttttgaatcttgggttggaaccaataaagggactcaaaacaaaaacaggcaaacaaaccaaaaggaaaaaaaaaaaacctacaaaactcaatagtgttattttgagaacttacttttttctttaaaaattttattattattattatcattgttgttgtttttttttgtttgtttgttttttgagacagggtttctctgtgtagccttagttgtcctggaactcacaaaagaaaacacacaaaataacaccaaacccagtacacaggaagcaggcctggctcacctgcagaactgcccgaacaaggcaagacagcctatgggacagcaaaacagggcacaaagaatgagagcccaggagagagagcaacagacagaagagacaaacccctaggcgccaccagagaatgattgccctggaggggagaaggtggagtactgggtgctgggaacacgccacacacacacactctgtgtgctggtgtttgctttgtgctaagccactgagctcaagtgcttgcggtttgcatccagaaactcatccagttgagccagtattacccccagagcaatgtgatggaaatcagcagtggtttgaatccagaaatataaaaacggcactttttttgctcacctatctattctagacttctgtgcactgatataggttccagggtctcaaatcccttaaaattctaatcaactgtgatgggccaggctggaagcagcgatgacacgcgtcctggaaaccctgccaagctgtcctgtaccgctgcttctgcgtgtgttgataattaatccttcggagcgggcgcacgaatgtgagaaattggctattttccgcttctgaatgatggctctgagccactacggcagcttttccatttcaaatctgactgtcaaaagtggtgcattaatcagtatttcgttagtttcaacatttccagaatccgcctccccaagtgcacatacaaagtgcagccatcctcagccgagaaggcaagggcggggccttgcttcaggtgacgactctttctttctctttgcaccactccatcctctgagaggctatggctgtgactccggagctcacttccctgcaaagtagtggctcttggctgagtacttccaacagggagacagaacaggaccggagggtggcagacggtgcctctatccaatggccccctgggtgacttccccagaagcactttattattattattattattattattattattattattattattattattggtgtggttagttgttttgagacaggattgctctttgtagcccaggctggccgtgaactcagaagtccacctgcctctgcatctggtgtgctgattctgatgtcaccaccagatgtgagcctccaccatcagactcacatccactacagacgacctgaatgtctcagccaagctgtatactagagaaagtggctactgttacaaaactcagtggagacacagctctagcagatgtaggtctggctgaccacataagcactgactagtgctggagacacactggtactgagcgacagctgtgctgagaaccatatctggctgaggtgctgcttgggtcaacctctcagagtgtccagcaggaggcagggcttccagacctactgatggatgagtctgggggctgggcaaactggacagcattattctgtggagaagccttagactgtcccccaaagtaaaagcaccccaaccagcctgttatacttcctccctcccactgtcagggccaccaacgcccaagagtgcaaaaggcacccaggaaagagaaagttaaaaaaacaagggaaggttgattttcctacttcctgctgcccctaactgccagacgaggcttctccccctgcccctcaagcaaccctcagactccgcccatgtccgtgccccgccccctcctctccaacagtgacgcgtgctgtgctggcatggctgccggtgggcaggtgatgcacatcccaggaacattttgcccgccccaactgcttcatcaccaagcctccagcaaagaggctcaggcagcctgaagcctcgctgcctggcatagcgcatttagagtaacagctcagctaatctcttccaagggaggcagagctgctcagcacagcaggagtggggcgagctctggaatattccagtgtggttctcccacaggcatctagggacagagaggagtgtgggggtgggggacaagctgagcctcacaggtgttcctaggctgatgcttaagt

SEQ ID NO: 81 - DNA, 16 bp, synthetic construct
  source 1..16: mol_type = other DNA
ggaatattggttcagt

SEQ ID NO: 82 - DNA, 16 bp, synthetic construct
  source 1..16: mol_type = other DNA
tgtcgaagctcaaccc

SEQ ID NO: 83 - DNA, 16 bp, synthetic construct
  source 1..16: mol_type = other DNA
atctatgtcactttgg

SEQ ID NO: 84 - DNA, 16 bp, synthetic construct
  source 1..16: mol_type = other DNA
gaatattggttcagta

SEQ ID NO: 85 - DNA, 16 bp, synthetic construct
  source 1..16: mol_type = other DNA
tgtatgtcgcacaggc

SEQ ID NO: 86 - DNA, 16 bp, synthetic construct
  source 1..16: mol_type = other DNA
acaattctatggtctc

SEQ ID NO: 87 - DNA, 20 bp, synthetic construct
  source 1..20: mol_type = other DNA
catggtcttcccctatcacc

SEQ ID NO: 88 - DNA, 20 bp, synthetic construct
  source 1..20: mol_type = other DNA
tgtcaatctccagcatcacc

SEQ ID NO: 89 - DNA, 22 bp, synthetic construct
  source 1..22: mol_type = other DNA
atcacctcaggacccagctcac

SEQ ID NO: 90 - RNA, 23 nt, synthetic construct
  source 1..23: mol_type = other RNA
  modified_base 22..23: mod_base = OTHER; note = thymine
ataaaatctacagtcataggatt

SEQ ID NO: 91 - DNA, 24 bp, synthetic construct
  source 1..24: mol_type = other DNA
caaaggagggacatgtatcaacac

SEQ ID NO: 92 - DNA, 20 bp, synthetic construct
  source 1..20: mol_type = other DNA
ctggcaatgtttcccagtga

SEQ ID NO: 93 - DNA, 24 bp, synthetic construct
  source 1..24: mol_type = other DNA
cccaatgggccacactgtctctgc